(19) United States
(12) Patent Application Publication
(10) Pub. No.: US 2026/0224550 A1
(43) Pub. Date: Aug. 6, 2026

(54) PDE4 DEGRADERS, PHARMACEUTICAL COMPOSITIONS, AND THERAPEUTIC APPLICATIONS

(71) Applicant: BioTheryX, Inc., San Diego, CA (US)

(72) Inventors: Aparajita Hoskote Chourasia, San Diego, CA (US); Paul E. Erdman, San Diego, CA (US); Leah M. Fung, San Diego, CA (US); Shenlin Huang, San Diego, CA (US); Imelda Lam, San Diego, CA (US); Arvind Shakya, San Diego, CA (US)

(21) Appl. No.: 19/152,438

(22) PCT Filed: Feb. 7, 2024
(86) PCT No.: PCT/US2024/014899

§ 371 Date: Jul. 30, 2025

Related U.S. Application Data
(60) Provisional application No. 63/483,740, filed on Feb. 7, 2023.

(51) Int. Cl. A61K 31/4545; A61K 31/496; A61K 31/513; C07D 401/14; C07D 471/04

(52) U.S. Cl. CPC A61K 31/4545; A61K 31/496; A61K 31/513; C07D 401/14; C07D 471/04

(57) ABSTRACT

Provided herein are phosphodiesterase 4 (PDE4) degraders, e.g., a compound of Formula (A), and pharmaceutical compositions thereof. Also provided herein are methods of their use for treating, preventing, or ameliorating one or more symptoms of a PDE4-mediated disease, disorder, or condition

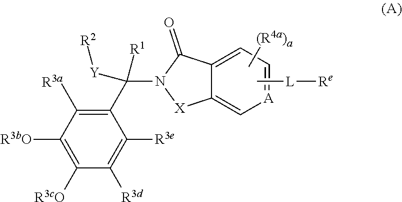

### CROSS REFERENCE TO RELATED APPLICATION

This application claims the benefit of the priority of U.S. Provisional Application No. 63/483,740, filed Feb. 7, 2023; the disclosure of which is incorporated herein by reference in its entirety.

### FIELD

Provided herein are phosphodiesterase 4 (PDE4) degraders and pharmaceutical compositions thereof. Also provided herein are methods of their use for treating, preventing, or ameliorating one or more symptoms of a PDE4-mediated disease, disorder, or condition.

### BACKGROUND

Aberrant protein function or protein imbalance is a hallmark of many disease states. For example, the functioning of the immune system is finely balanced by the activities of pro-inflammatory and anti-inflammatory mediators or cytokines. Some cytokines promote inflammation (pro-inflammatory cytokines), whereas other cytokines suppress the activity of the pro-inflammatory cytokines (anti-inflammatory cytokines). For example, interleukin-4 (IL-4), interleukin-10 (IL-10), and interleukin-13 (IL-13) are potent activators of B lymphocytes, and also act as anti-inflammatory agents. They are anti-inflammatory cytokines by virtue of their ability to suppress genes for pro-inflammatory cytokines, such as interleukin-1 (IL-1), a tumor necrosis factor (TNF), and chemokines.

Unregulated activities of these mediators can lead to the development of serious inflammatory conditions. For example, autoimmune diseases arise when immune system cells (lymphocytes and macrophages) become sensitized against the “self.” Lymphocytes as well as macrophages are usually under control in this system. However, a misdirection of the system toward the body's own tissues may happen in response to still unexplained triggers. One hypothesis is that lymphocytes recognize an antigen which mimics the “self” and a cascade of activation of different components of the immune system takes place, ultimately leading to tissue destruction. Genetic predisposition has also been postulated to be responsible for autoimmune disorders.

A phosphodiesterase 4 (PDE4) is involved in the cytokine production of inflammatory cells, angiogenesis, and the functional properties of other cell types such as keratinocytes, in part, through degradation of cyclic adenosine monophosphate (cAMP). cAMP is an important second messenger that regulates inflammatory responses. Accordingly, inhibitors of PDE4 may block the synthesis of several pro-inflammatory cytokines and chemokines, such as tumor necrosis factor alpha (TNF-α), interleukin-23 (IL-23), chemokine ligand 9 (CXCL9, also known as monokine induced by interferon gamma (MIG)), and chemokine ligand 10 (CXCL10, also known as interferon gamma-induced protein 10 (IP-10)) in multiple cell types, and may interfere with the production of leukotriene B4, inducible nitric oxide synthase, and matrix metalloproteinases. This interference reduces certain inflammatory processes, such as dendritic cell infiltration, epidermal skin thickening, and joint destruction, for example, in psoriasis and other inflammatory and/or autoimmune diseases such as arthritis, ankylosing spondylitis, osteoarthritis, rheumatoid arthritis, Behcet's disease, inflammatory bowel diseases (e.g., Crohn's disease and ulcerative colitis), psoriasis, atopic dermatitis, and contact dermatitis.

Psoriasis is an autoimmune skin disease caused by pro-inflammatory cytokines, interferon gamma (IFN-γ) and TNF-α. The psoriatic immune response involves monocytes, dendritic cells, neutrophils, and T cells, which all contribute to aberrant keratinocyte proliferation. PDE4 inhibition may reduce production of multiple mediators, including TNF-α, IFN-7, CXCL9, CXCL10, interleukin-2 (IL-2), interleukin-12 (IL-12), IL-23, macrophage inflammatory protein-1-alpha (MIP-la), monocyte chemoattractant protein-1 (MCP1), and granulocyte macrophage-colony stimulating factor (GM-CSF) from PBMCs. Thus, there is a continued need for small molecule PDE4 modulators as an effective therapy for treating an inflammatory disease.

### SUMMARY OF THE DISCLOSURE

Provided herein is a compound of Formula (A):

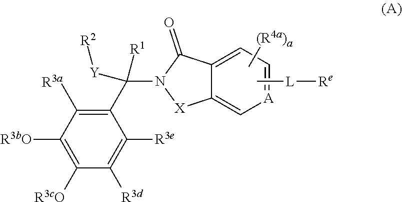

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
A is C(R⁴) or N;
L is a linker;
X is —CH₂— or —C(O)—;
Y is C₁₋₆ alkylene or C₃₋₁₀ cycloalkylene;
R¹ is hydrogen or deuterium;
R² is cyano or —S(O₂)R^{2a};
R⁴ is hydrogen or R⁴;
R^{2a} is (i) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (ii) —OR^{1a} or —NR^{1b}R^{1c};
R^{3a}, R^{3d}, and R^{3e} are each independently (i) hydrogen, deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{3b} and R^{3c} are each independently C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
each R^{4a} is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{e} is

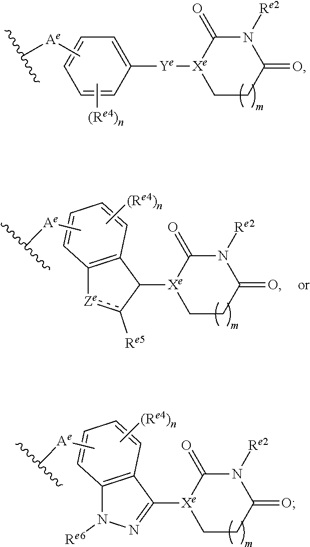

wherein:
   each A^{e} is independently (i) C₁₋₆ alkylene, C₂₋₆ alkenylene, C₂₋₆ alkynylene, C₃₋₁₀ cycloalkylene, C₆₋₁₄ arylene, heteroarylene, or heterocyclylene;
or (ii) —O— or —NHCH₂CH₂—;
   X^{e} is C(R^{e1}) or N;
   Y^{e} is a bond, C₁₋₆ alkylene, —O—, —S—, —S(O)—, —S(O₂)—, or —N(R^{e3})—;
   Z^{e} is —CH═, —CH₂—, —N═, or —N(R^{e6})—;
   R^{e1} is hydrogen, deuterium, halo, or C₁₋₆ alkyl;
   each R^{e2}, R^{e3}, and R^{e6} is independently hydrogen or C₁₋₆ alkyl;
   each R^{e4} is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —R^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(N^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
   R^{e1} is hydrogen or oxo;
   each m is independently an integer of 0, 1, or 2; and
   each n is independently an integer of 0, 1, 2, 3, or 4;
each R^{1a}, R^{1b}, R^{1c}, and R^{1d} is independently hydrogen, deuterium, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; and
a is an integer of 0, 1, 2, 3, or 4;
wherein each alkyl, alkylene, heteroalkyl, alkenyl, alkenylene, alkynyl, alkynylene, cycloalkyl, cycloalkylene, aryl, arylene, aralkyl, heteroaryl, heteroarylene, heterocyclyl, and heterocyclylene is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})R^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{a})OR^{d}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{c}, —C(NR^{c})NR^{f}R^{g}, —C(S)R^{c}, —C(S)OR^{c}, —C(S)NR^{f}R^{g}, —OR^{c}, —OC(O)R^{c}, —OC(O)OR^{c}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OP(O)(OR^{f})OR^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

Also provided herein is a compound of Formula (I):

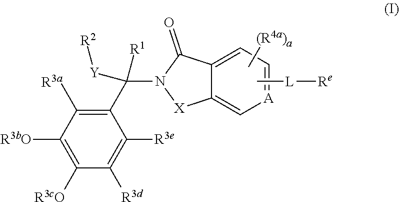

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
L is a linker;
X is —CH₂— or —C(O)—;
Y is C₁₋₆ alkylene or C₃₋₁₀ cycloalkylene;
R¹ is hydrogen or deuterium;
R² is cyano or —S(O₂)R^{2a};
R^{2a} is (i) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (ii) —OR^{1a} or —NR^{1b}R^{1c};
R^{3a}, R^{3d}, and R^{3e} are each independently (i) hydrogen, deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1c}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{3b} and R^{3c} are each independently C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
each R^{4a} is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)²NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{e} is

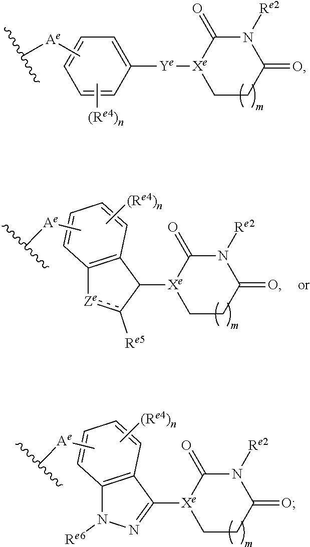

wherein:
   each A^{e} is independently (i) C₁₋₆ alkylene, C₂₋₆ alkenylene, C₂₋₆ alkynylene, C₃₋₁₀ cycloalkylene, C₆₋₁₄ arylene, heteroarylene, or heterocyclylene; or (ii) —O— or —NHCH₂CH₂—;
   X^{e} is C(R^{e1}) or N;
   Y^{e} is a bond, C₁₋₆ alkylene, —O—, —S—, —S(O)—, —S(O₂)—, or —N(R^{e3})—;
   Z^{e} is —CH═, —CH₂—, —N═, or —N(R^{e6})—;
   R^{e1} is hydrogen, deuterium, halo, or C₁₋₆ alkyl;
   each R^{e2}, R^{e3}, and R^{e6} is independently hydrogen or C₁₋₆ alkyl;
   each R^{c4} is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
   R^{e5} is hydrogen or oxo;
   each m is independently an integer of 0, 1, or 2; and
   each n is independently an integer of 0, 1, 2, 3, or 4;
each R^{1a}, R^{1b}, R^{1c}, and R^{1d} is independently hydrogen, deuterium, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; and
a is an integer of 0, 1, 2, 3, or 4;
wherein each alkyl, alkylene, heteroalkyl, alkenyl, alkenylene, alkynyl, alkynylene, cycloalkyl, cycloalkylene, aryl, arylene, aralkyl, heteroaryl, heteroarylene, heterocyclyl, and heterocyclylene is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{a})OR^{d}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{C}(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OP(O)(OR^{f})OR^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

Additionally, provided herein is a pharmaceutical composition comprising a compound of Formula (A) or (I), or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof; and a pharmaceutically acceptable excipient.

Furthermore, provided herein is a method of treating, preventing, or ameliorating one or more symptoms of a disorder, disease, or condition mediated by a phosphodiesterase 4 (PDE4) in a subject, comprising administering to the subject in need thereof a therapeutically effective amount of a compound of Formula (A) or (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

Provided herein is a method of treating, preventing, or ameliorating one or more symptoms of an inflammatory disease in a subject, comprising administering to the subject in need thereof a therapeutically effective amount of a compound of Formula (A) or (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

Provided herein is a method of inhibiting the activity of a PDE4, comprising contacting the PDE4 with an effective amount of a compound of Formula (A) or (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

Provided herein is a method of inducing degradation of a PDE4, comprising contacting the PDE4 with an effective amount of a compound of Formula (A) or (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

### DETAILED DESCRIPTION

To facilitate understanding of the disclosure set forth herein, a number of terms are defined below.

Generally, the nomenclature used herein and the laboratory procedures in organic chemistry, medicinal chemistry, biochemistry, biology, and pharmacology described herein are those well-known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject. In one embodiment, the subject is a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The terms “alleviate” and “alleviating” refer to easing or reducing one or more symptoms (e.g., pain) of a disorder, disease, or condition. The terms can also refer to reducing adverse effects associated with an active ingredient. Sometimes, the beneficial effects that a subject derives from a prophylactic or therapeutic agent do not result in a cure of the disorder, disease, or condition.

The term “contacting” or “contact” is meant to refer to bringing together of a therapeutic agent and a biological molecule (e.g., a protein, enzyme, RNA, or DNA), cell, or tissue such that a physiological and/or chemical effect takes place as a result of such contact. Contacting can take place in vitro, ex vivo, or in vivo. In one embodiment, a therapeutic agent is contacted with a biological molecule in vitro to determine the effect of the therapeutic agent on the biological molecule. In another embodiment, a therapeutic agent is contacted with a cell in cell culture (in vitro) to determine the effect of the therapeutic agent on the cell. In yet another embodiment, the contacting of a therapeutic agent with a biological molecule, cell, or tissue includes the administration of a therapeutic agent to a subject having the biological molecule, cell, or tissue to be contacted.

The term “therapeutically effective amount” or “effective amount” is meant to include the amount of a compound that, when administered, is sufficient to prevent development of, or alleviate to some extent, one or more of the symptoms of the disorder, disease, or condition being treated. The term “therapeutically effective amount” or “effective amount” also refers to the amount of a compound that is sufficient to elicit a biological or medical response of a biological molecule (e.g., a protein, enzyme, RNA, or DNA), cell, tissue, system, animal, or human, which is being sought by a researcher, veterinarian, medical doctor, or clinician.

The term “IC₅₀” or “EC₅₀” refers to an amount, concentration, or dosage of a compound that is required for 50% inhibition of a maximal response in an assay that measures such a response.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of a subject (e.g., a human) without excessive toxicity, irritation, allergic response, immunogenicity, or other problems or complications, and commensurate with a reasonable benefit/risk ratio. See, e.g., Remington: The Science and Practice of Pharmacy, 23rd ed.; Adejare Ed.; Academic Press, 2020; Handbook of Pharmaceutical Excipients, 9th ed.; Sheskey et al., Eds.; Pharmaceutical Press, 2020; Handbook of Pharmaceutical Additives, 3rd ed.; Ash and Ash Eds.; Synapse Information Resources, 2007; Pharmaceutical Preformulation and Formulation, 2nd ed.; Gibson Ed.; CRC Press, 2009.

The term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 25%, 20%, 15%, 10%, 9%, 8%, 7%, 6%, 5%, 4%, 3%, 2%, 1%, 0.5%, or 0.05% of a given value or range.

The term “alkyl” refers to a linear or branched saturated monovalent hydrocarbon radical, wherein the alkyl is optionally substituted with one or more substituents Q as described herein. For example, C₁₋₆ alkyl refers to a linear saturated monovalent hydrocarbon radical of 1 to 6 carbon atoms or a branched saturated monovalent hydrocarbon radical of 3 to 6 carbon atoms. In certain embodiments, the alkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (including all isomeric forms, e.g., n-propyl and isopropyl), butyl (including all isomeric forms, e.g., n-butyl, isobutyl, sec-butyl, and t-butyl), pentyl (including all isomeric forms, e.g., n-pentyl, isopentyl, sec-pentyl, neopentyl, and tert-pentyl), and hexyl (including all isomeric forms, e.g., n-hexyl, isohexyl, and sec-hexyl).

The terms “alkylene” and “alkanediyl” are used interchangeably herein in reference to a linear or branched saturated divalent hydrocarbon radical, wherein the alkanediyl is optionally be substituted with one or more substituents Q as described herein. For example, C₁₋₆ alkanediyl refers to a linear saturated divalent hydrocarbon radical of 1 to 6 carbon atoms or a branched saturated divalent hydrocarbon radical of 3 to 6 carbon atoms. In certain embodiments, the alkanediyl is a linear saturated divalent hydrocarbon radical that has 1 to 30 (C₁₋₃₀), 1 to 20 (C₁₋₂₀), 1 to 15 (C₁₋₁₅), 1 to 10 (C₁₋₁₀), or 1 to 6 (C₁₋₆) carbon atoms, or branched saturated divalent hydrocarbon radical of 3 to 30 (C₃₋₃₀), 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkanediyl groups are also referred as “lower alkanediyl.” Examples of alkanediyl groups include, but are not limited to, methanediyl, ethanediyl (including all isomeric forms, e.g., ethane-1,1-diyl and ethane-1,2-diyl), propanediyl (including all isomeric forms, e.g., propane-1,1-diyl, propane-1,2-diyl, and propane-1,3-diyl), butanediyl (including all isomeric forms, e.g., butane-1,1-diyl, butane-1,2-diyl, butane-1,3-diyl, and butane-1,4-diyl), pentanediyl (including all isomeric forms, e.g., pentane-1,1-diyl, pentane-1,2-diyl, pentane-1,3-diyl, and pentane-1,5-diyl), and hexanediyl (including all isomeric forms, e.g., hexane-1,1-diyl, hexane-1,2-diyl, hexane-1,3-diyl, and hexane-1,6-diyl). Examples of substituted alkanediyl groups include, but are not limited to, —C(O)CH₂—, —C(O)(CH₂)₂—, —C(O)(CH₂)₃—, —C(O)(CH₂)₄—, —C(O)(CH₂)₅—, —C(O)(CH₂)₆—, —C(O)(CH₂)₇—, —C(O)(CH₂)₈—, —C(O)(CH₂)₉—, —C(O)(CH₂)₁₀—, —C(O)CH₂C(O)—, —C(O)(CH₂)₂C(O)—, —C(O)(CH₂)₃C(O)—, —C(O)(CH₂)₄C(O)—, or —C(O)(CH₂)₅C(O)—.

The term “heteroalkyl” refers to a linear or branched saturated monovalent hydrocarbon radical that contains one or more heteroatoms on its main chain, each independently selected from O, S, and N. The heteroalkyl is optionally substituted with one or more substituents Q as described herein. For example, C₁₋₆ heteroalkyl refers to a linear saturated monovalent hydrocarbon radical of 1 to 6 carbon atoms or a branched saturated monovalent hydrocarbon radical of 3 to 6 carbon atoms. In certain embodiments, the heteroalkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ heteroalkyl groups are also referred as “lower heteroalkyl.” Examples of heteroalkyl groups include, but are not limited to, —OCH₃, —OCH₂CH₃, —CH₂OCH₃, —NHCH₃, —ONHCH₃, —NHOCH₃, —SCH₃, —CH₂NHCH₂CH₃, and —NHCH₂CH₂CH₃. Examples of substituted heteroalkyl groups include, but are not limited to, —CH₂NHC(O)CH₃ and —NHC(O)CH₂CH₃.

The terms “heteroalkylene” and “heteroalkanediyl” are used interchangeably herein in reference to a linear or branched saturated divalent hydrocarbon radical that contains one or more heteroatoms in its main chain, each independently selected from O, S, and N. The heteroalkylene is optionally substituted with one or more substituents Q as described herein. For example, C₁₋₆ heteroalkylene refers to a linear saturated divalent hydrocarbon radical of 1 to 6 carbon atoms or a branched saturated divalent hydrocarbon radical of 3 to 6 carbon atoms. In certain embodiments, the heteroalkylene is a linear saturated divalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ heteroalkylene groups are also referred as “lower heteroalkylene.” Examples of heteroalkylene groups include, but are not limited to, —CH₂O—, —CH₂CH₂O—, —CH₂CH₂CH₂O—, —(CH₂)₄O—, —(CH₂)₅O—, —(CH₂)₆O—, —(CH₂)₇O—, —(CH₂)₈O—, —(CH₂)₉O—, —(CH₂)₁₀O—, —CH₂OCH₂—, —CH₂CH₂O—, —(CH₂CH₂O)₂—, —(CH₂CH₂O)₃—, —(CH₂CH₂O)₄—, —(CH₂CH₂O)₅—, —CH₂NH—, —CH₂NHCH₂—, —CH₂CH₂NH—, —CH₂CH₂CH₂NH—, —(CH₂)₄NH—, —CH₂S—, —CH₂SCH₂—, and —CH₂CH₂S—. Examples of substituted heteroalkylene groups include, but are not limited to, —C(O)CH₂O—, —C(O)(CH₂)₂O—, —C(O)CH₂CH₂CH₂O—, —C(O)CH₂CH₂CH₂CH₂O—, —C(O)(CH₂)₅O—, —C(O)(CH₂)₆O—, —C(O)(CH₂)₇O—, —C(O)(CH₂)₈O—, —C(O)(CH₂)₉O—, —C(O)(CH₂)₁₀O—, —C(O)CH₂OCH₂CH₂O—, —C(O)CH₂O(CH₂CH₂O)₂—, —C(O)CH₂O(CH₂CH₂O)₃—, —C(O)CH₂O(CH₂CH₂O)₄, —C(O)CH₂O(CH₂CH₂O)₅—, —CH₂NHC(O)CH₂—, —CH₂CH₂C(O)NH—, —CH₂N(CH₃)—, —(CH₂)₂N(CH₃)—, —(CH₂)₃N(CH₃)—, or —(CH₂)₄N(CH₃)—.

The term “alkenyl” refers to a linear or branched monovalent hydrocarbon radical, which contains one or more, in one embodiment, one, two, three, or four, in another embodiment, one, carbon-carbon double bond(s). The alkenyl is optionally substituted with one or more substituents Q as described herein. The term “alkenyl” embraces radicals having a “cis” or “trans” configuration or a mixture thereof, or alternatively, a “Z” or “E” configuration or a mixture thereof, as appreciated by those of ordinary skill in the art. For example, C₂₋₆ alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenyl groups include, but are not limited to, ethenyl, propenyl (including all isomeric forms, e.g., propen-1-yl, propen-2-yl, and allyl), and butenyl (including all isomeric forms, e.g., buten-1-yl, buten-2-yl, buten-3-yl, and 2-buten-1-yl).

The terms “alkenylene” and “alkenediyl” are used interchangeably herein in reference to a linear or branched divalent hydrocarbon radical, which contains one or more, in one embodiment, one, two, three, or four, in another embodiment, one, carbon-carbon double bond(s). The alkenediyl is optionally substituted with one or more substituents Q as described herein. The term “alkenediyl” embraces radicals having a “cis” or “trans” configuration or a mixture thereof, or alternatively, a “Z” or “E” configuration or a mixture thereof, as appreciated by those of ordinary skill in the art. For example, C₂₋₆ alkenediyl refers to a linear unsaturated divalent hydrocarbon radical of 2 to 6 carbon atoms or a branched unsaturated divalent hydrocarbon radical of 3 to 6 carbon atoms. In certain embodiments, the alkenediyl is a linear divalent hydrocarbon radical of 2 to 30 (C₂₋₃₀), 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 3 to 30 (C₃₋₃₀), 3 to 20 (C₃₋₂₀), 3 to 15 (C₃₋₁₅), 3 to 10 (C₃₋₁₀), or 3 to 6 (C₃₋₆) carbon atoms. Examples of alkenediyl groups include, but are not limited to, ethenediyl (including all isomeric forms, e.g., ethene-1,1-diyl and ethene-1,2-diyl), propanediyl (including all isomeric forms, e.g., 1-propene-1,1-diyl, 1-propene-1,2-diyl, and 1-propene-1,3-diyl), butenediyl (including all isomeric forms, e.g., 1-butene-1,1-diyl, 1-butene-1,2-diyl, and 1-butene-1,4-diyl), pentanediyl (including all isomeric forms, e.g., 1-pentene-1,1-diyl, 1-pentene-1,2-diyl, and 1-pentene-1,5-diyl), and hexanediyl (including all isomeric forms, e.g., 1-hexene-1,1-diyl, 1-hexene-1,2-diyl, 1-hexene-1,3-diyl, 1-hexene-1,4-diyl, 1-hexene-1,5-diyl, and 1-hexene-1,6-diyl).

The terms “heteroalkenylene” and “heteroalkenediyl” are used interchangeably herein in reference to a linear or branched divalent hydrocarbon radical, which contains one or more, in one embodiment, one, two, three, or four, in another embodiment, one, carbon-carbon double bond(s), and which contains one or more heteroatoms each independently selected from O, S, and N in the hydrocarbon chain. The heteroalkenylene is optionally substituted with one or more substituents Q as described herein. The term “heteroalkenylene” embraces radicals having a “cis” or “trans” configuration or a mixture thereof, or alternatively, a “Z” or “E” configuration or a mixture thereof, as appreciated by those of ordinary skill in the art. For example, C₂₋₆ heteroalkenylene refers to a linear unsaturated divalent hydrocarbon radical of 2 to 6 carbon atoms or a branched unsaturated divalent hydrocarbon radical of 3 to 6 carbon atoms. In certain embodiments, the heteroalkenylene is a linear divalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of heteroalkenylene groups include, but are not limited to, —CH═CHO—, —CH═CHOCH₂—, —CH═CHCH₂O—, —CH═CHS—, —CH═CHSCH₂—, —CH═CHCH₂S—, or —CH═CHCH₂NH—.

The term “alkynyl” refers to a linear or branched monovalent hydrocarbon radical, which contains one or more, in one embodiment, one, two, three, or four, in another embodiment, one, carbon-carbon triple bond(s). An alkynyl group does not contain a carbon-carbon double bond. The alkynyl is optionally substituted with one or more substituents Q as described herein. For example, C₂₋₆ alkynyl refers to a linear unsaturated monovalent hydrocarbon radical of 2 to 6 carbon atoms or a branched unsaturated monovalent hydrocarbon radical of 4 to 6 carbon atoms. In certain embodiments, the alkynyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 4 to 20 (C₄₋₂₀), 4 to 15 (C₄₋₁₅), 4 to 10 (C₄₋₁₀), or 4 to 6 (C₄₋₆) carbon atoms. Examples of alkynyl groups include, but are not limited to, ethynyl (—C≡CH), propynyl (including all isomeric forms, e.g., 1-propynyl (—C≡CCH₃) and propargyl (—CH₂C≡CH)), butynyl (including all isomeric forms, e.g., 1-butyn-1-yl and 2-butyn-1-yl), pentynyl (including all isomeric forms, e.g., 1-pentyn-1-yl and 1-methyl-2-butyn-1-yl), and hexynyl (including all isomeric forms, e.g., 1-hexyn-1-yl and 2-hexyn-1-yl).

The terms “alkynylene” and “alkynediyl” are used interchangeably herein in reference to a linear or branched divalent hydrocarbon radical, which contains one or more, in one embodiment, one, two, three, or four, in another embodiment, one, carbon-carbon triple bond(s). An alkynylene group does not contain a carbon-carbon double bond. The alkynediyl is optionally substituted with one or more substituents Q as described herein. For example, C₂₋₆ alkynediyl refers to a linear unsaturated divalent hydrocarbon radical of 2 to 6 carbon atoms or a branched unsaturated divalent hydrocarbon radical of 4 to 6 carbon atoms. In certain embodiments, the alkynediyl is a linear divalent hydrocarbon radical of 2 to 30 (C₂₋₃₀), 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 4 to 30 (C₄₋₃₀), 4 to 20 (C₄₋₂₀), 4 to 15 (C₄₋₁₅), 4 to 10 (C₄₋₁₀), or 4 to 6 (C₄₋₆) carbon atoms. Examples of alkynediyl groups include, but are not limited to, ethynediyl, propynediyl (including all isomeric forms, e.g., 1-propyne-1,3-diyl and 1-propyne-3,3-diyl), butynediyl (including all isomeric forms, e.g., 1-butyne-1,3-diyl, 1-butyne-1,4-diyl, and 2-butyne-1,1-diyl), pentynediyl (including all isomeric forms, e.g., 1-pentyne-1,3-diyl, 1-pentyne-1,4-diyl, and 2-pentyne-1,1-diyl), and hexynediyl (including all isomeric forms, e.g., 1-hexyne-1,3-diyl, 1-hexyne-1,4-diyl, and 2-hexyne-1,1-diyl).

The terms “heteroalkynylene” and “heteroalkynediyl” are used interchangeably herein in reference to a linear or branched divalent hydrocarbon radical, which contains one or more, in one embodiment, one, two, three, or four, in another embodiment, one, carbon-carbon triple bond(s), and which contains one or more heteroatoms in its main chain, each independently selected from O, S, and N. A heteroalkynylene group does not contain a carbon-carbon double bond. The heteroalkynylene is optionally substituted with one or more substituents Q as described herein. For example, C₂₋₆ heteroalkynylene refers to a linear unsaturated divalent hydrocarbon radical of 2 to 6 carbon atoms or a branched unsaturated divalent hydrocarbon radical of 4 to 6 carbon atoms. In certain embodiments, the heteroalkynylene is a linear divalent hydrocarbon radical of 2 to 30 (C₂₋₃₀), 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 4 to 30 (C₄₋₃₀), 4 to 20 (C₄₋₂₀), 4 to 15 (C₄₋₁₅), 4 to 10 (C₄₋₁₀), or 4 to 6 (C₄₋₆) carbon atoms. Examples of heteroalkynylene groups include, but are not limited to, —C≡CCH₂O—, —C≡CCH₂S—, or —C≡CCH₂NH—.

The term “cycloalkyl” refers to a cyclic monovalent hydrocarbon radical, which is optionally substituted with one or more substituents Q as described herein. In one embodiment, the cycloalkyl is a saturated or unsaturated but non-aromatic, and/or bridged or non-bridged, and/or fused bicyclic group. In certain embodiments, the cycloalkyl has from 3 to 20 (C₃₋₂₀), from 3 to 15 (C₃₋₁₅), from 3 to 10 (C₃₋₁₀), or from 3 to 7 (C₃₋₇) carbon atoms. In one embodiment, the cycloalkyl is monocyclic. In another embodiment, the cycloalkyl is bicyclic. In yet another embodiment, the cycloalkyl is tricyclic. In still another embodiment, the cycloalkyl is polycyclic. Examples of cycloalkyl groups include, but are not limited to, cyclopropyl, cyclobutyl, cyclopentyl, cyclopentenyl, cyclohexyl, cyclohexenyl, cyclohexadienyl, cycloheptyl, cycloheptenyl, bicyclo[1.1.1]pentyl, bicyclo[2.1.1]hexyl, bicyclo[2.2.1]heptyl, bicyclo[2.2.2]-octyl, decalinyl, and adamantyl.

The terms “cycloalkylene” and “cycloalkanediyl” are used interchangeably herein in reference to a cyclic divalent hydrocarbon radical, which may be optionally substituted with one or more substituents Q as described herein. In one embodiment, cycloalkanediyl groups may be saturated or unsaturated but non-aromatic, and/or bridged, and/or non-bridged, and/or fused bicyclic groups. In certain embodiments, the cycloalkanediyl has from 3 to 30 (C₃₋₃₀), 3 to 20 (C₃₋₂₀), from 3 to 15 (C₃₋₁₅), from 3 to 10 (C₃₋₁₀), or from 3 to 7 (C₃₋₇) carbon atoms. Examples of cycloalkanediyl groups include, but are not limited to, cyclopropanediyl (including all isomeric forms, e.g., cyclopropane-1,1-diyl and cyclopropane-1,2-diyl), cyclobutanediyl (including all isomeric forms, e.g., cyclobutane-1,1-diyl, cyclobutane-1,2-diyl, and cyclobutane-1,3-diyl), cyclopentanediyl (including all isomeric forms, e.g., cyclopentane-1,1-diyl, cyclopentane-1,2-diyl, and cyclopentane-1,3-diyl), cyclohexanediyl (including all isomeric forms, e.g., cyclohexane-1,1-diyl, cyclohexane-1,2-diyl, cyclohexane-1,3-diyl, and cyclohex-1,4-diyl), cycloheptanediyl (including all isomeric forms, e.g., cycloheptane-1,1-diyl, cycloheptane-1,2-diyl, cycloheptane-1,3-diyl, and cycloheptane-1,4-diyl), decalinediyl (including all isomeric forms, e.g., decaline-1,1-diyl, decaline-1,2-diyl, and decaline-1,8-diyl), and adamantdiyl (including all isomeric forms, e.g., adamant-1,2-diyl, adamant-1,3-diyl, and adamant-1,8-diyl).

The term “aryl” refers to a monovalent monocyclic aromatic hydrocarbon radical and/or monovalent polycyclic aromatic hydrocarbon radical that contain at least one aromatic carbon ring. In certain embodiments, the aryl has from 6 to 20 (C₆₋₂₀), from 6 to 15 (C₆₋₁₅), or from 6 to 10 (C₆₋₁₀) ring carbon atoms. Examples of aryl groups include, but are not limited to, phenyl, naphthyl, fluorenyl, azulenyl, anthryl, phenanthryl, pyrenyl, biphenyl, and terphenyl. The aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl (tetralinyl). In one embodiment, the aryl is monocyclic. In another embodiment, the aryl is bicyclic. In yet another embodiment, the aryl is tricyclic. In still another embodiment, the aryl is polycyclic. In certain embodiments, the aryl is optionally substituted with one or more substituents Q as described herein.

The terms “arylene” and “arenediyl” are used interchangeably herein in reference to a divalent monocyclic aromatic hydrocarbon radical or divalent polycyclic aromatic hydrocarbon radical that contains at least one aromatic hydrocarbon ring. In certain embodiments, the arylene has from 6 to 20 (C₆₋₂₀), from 6 to 15 (C₆₋₁₅), or from 6 to 10 (C₆₋₁₀) ring atoms. Examples of arylene groups include, but are not limited to, phenylene (including all isomeric forms, e.g., phen-1,2-diyl, phen-1,3-diyl, and phen-1,4-diyl), naphthylene (including all isomeric forms, e.g., naphth-1,2-diyl, naphth-1,3-diyl, and naphth-1,8-diyl), fluorenylene (including all isomeric forms, e.g., fluoren-1,2-diyl, fluoren-1,3-diyl, and fluoren-1,8-diyl), azulenylene (including all isomeric forms, e.g., azulen-1,2-diyl, azulen-1,3-diyl, and azulen-1,8-diyl), anthrylene (including all isomeric forms, e.g., anthr-1,2-diyl, anthr-1,3-diyl, and anthr-1,8-diyl), phenanthrylene (including all isomeric forms, e.g., phenanthr-1,2-diyl, phenanthr-1,3-diyl, and phenanthr-1,8-diyl), pyrenylene (including all isomeric forms, e.g., pyren-1,2-diyl, pyren-1,3-diyl, and pyren-1,8-diyl), biphenylene (including all isomeric forms, e.g., biphen-2,3-diyl, biphen-3,4′-diyl, and biphen-4,4′-diyl), and terphenylene (including all isomeric forms, e.g., terphen-2,3-diyl, terphen-3,4′-diyl, and terphen-4,4′-diyl). Arylene also refers to bicyclic or tricyclic carbon rings, where one of the rings is aromatic and the others of which may be saturated, partially unsaturated, or aromatic, for example, dihydronaphthylene (including all isomeric forms, e.g., dihydronaphth-1,2-diyl and dihydronaphth-1,8-diyl), indenylene (including all isomeric forms, e.g., inden-1,2-diyl, inden-1,5-diyl, and inden-1,7-diyl), indanylene (including all isomeric forms, e.g., indan-1,2-diyl, indan-1,5-diyl, and indan-1,7-diyl), or tetrahydronaphthylene (tetralinylene) (including all isomeric forms, e.g., tetrahydronaphth-1,2-diyl, tetrahydronaphth-1,5-diyl, and tetrahydronaphth-1,8-diyl). In certain embodiments, arylene is optionally substituted with one or more substituents Q as described herein.

The term “aralkyl” or “arylalkyl” refers to a monovalent alkyl group substituted with one or more aryl groups. In certain embodiments, the aralkyl has from 7 to 30 (C₇₋₃₀), from 7 to 20 (C₇₋₂₀), or from 7 to 16 (C₇₋₁₆) carbon atoms. Examples of aralkyl groups include, but are not limited to, benzyl, phenylethyl (including all isomeric forms, e.g., 1-phenylethyl and 2-phenylethyl), and phenylpropyl (including all isomeric forms, e.g., 1-phenylpropyl, 2-phenylpropyl, and 3-phenylpropyl). In certain embodiments, the aralkyl is optionally substituted with one or more substituents Q as described herein.

The term “aralkylene” or “arylalkylene” refers to a divalent alkyl group substituted with one or more aryl groups. In certain embodiments, the aralkylene has from 7 to 30 (C₇₋₃), from 7 to 20 (C₇₋₂₀), or from 7 to 16 (C₇₋₁₆) carbon atoms. Examples of aralkylene groups include, but are not limited to, benzylene (including all isomeric forms, e.g., phenylmethdiyl), phenylethylene (including all isomeric forms, e.g., 2-phenyl-ethan-1,1-diyl and 2-phenyl-ethan-1,2-diyl), and phenylpropylene (including all isomeric forms, e.g., 3-phenyl-propan-1,1-diyl, 3-phenyl-propan-1,2-diyl, and 3-phenyl-propan-1,3-diyl). In certain embodiments, the aralkylene is optionally substituted with one or more substituents Q as described herein.

The term “heteroaryl” refers to a monovalent monocyclic aromatic group or monovalent polycyclic aromatic group that contain at least one aromatic ring, wherein at least one aromatic ring contains one or more heteroatoms, each independently selected from O, S, and N, in the ring. For a heteroaryl group containing a heteroaromatic ring and a nonaromatic heterocyclic ring, the heteroaryl group is not bonded to the rest of a molecule through its nonaromatic heterocyclic ring. Each ring of a heteroaryl group can contain one or two O atoms, one or two S atoms, and/or one to four N atoms; provided that the total number of heteroatoms in each ring is four or less and each ring contains at least one carbon atom. In certain embodiments, the heteroaryl has from 5 to 20, from 5 to 15, or from 5 to 10 ring atoms. In one embodiment, the heteroaryl is monocyclic. Examples of monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. In another embodiment, the heteroaryl is bicyclic. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyrindyl (including all isomeric forms, e.g., furo[2,3-b]pyridinyl, furo[2,3-c]pyridinyl, furo[3,2-b]pyridinyl, furo[3,2-c]pyridinyl, furo[3,4-b]pyridinyl, and furo[3,4-c]pyridinyl), imidazopyridinyl (including all isomeric forms, e.g., imidazo[1,2-a]pyridinyl, imidazo[4,5-b]pyridinyl, and imidazo[4,5-c]pyridinyl), imidazothiazolyl (including all isomeric forms, e.g., imidazo[2,1-b]thiazolyl and imidazo[4,5-d]thiazolyl), indazolyl, indolizinyl, indolyl, isobenzofuranyl, isobenzothienyl (i.e., benzo[c]thienyl), isoindolyl, isoquinolinyl, naphthyridinyl (including all isomeric forms, e.g., 1,5-naphthyridinyl, 1,6-naphthyridinyl, 1,7-naphthyridinyl, and 1,8-naphthyridinyl), oxazolopyridinyl (including all isomeric forms, e.g., oxazolo[4,5-b]pyridinyl, oxazolo[4,5-c]pyridinyl, oxazolo[5,4-b]pyridinyl, and oxazolo[5,4-c]pyridinyl), phthalazinyl, pteridinyl, purinyl, pyrrolopyridyl (including all isomeric forms, e.g., pyrrolo[2,3-b]pyridinyl, pyrrolo[2,3-c]pyridinyl, pyrrolo[3,2-b]pyridinyl, and pyrrolo[3,2-c]pyridinyl), quinolinyl, quinoxalinyl, quinazolinyl, thiadiazolopyrimidyl (including all isomeric forms, e.g., [1,2,5]thiadiazolo[3,4-d]pyrimidinyl and [1,2,3]thiadiazolo[4,5-d]pyrimidinyl), and thieno-pyridyl (including all isomeric forms, e.g., thieno[2,3-b]pyridinyl, thieno[2,3-c]pyridinyl, thieno[3,2-b]pyridinyl, and thieno[3,2-c]pyridinyl). In yet another embodiment, the heteroaryl is tricyclic. Examples of tricyclic heteroaryl groups include, but are not limited to, acridinyl, benz-indolyl, carbazolyl, dibenzofuranyl, perimidinyl, phenanthrolinyl, phenanthridinyl (including all isomeric forms, e.g., 1,5-phenanthrolinyl, 1,6-phenanthrolinyl, 1,7-phenanthrolinyl, 1,9-phenanthrolinyl, and 2,10-phenanthrolinyl), phenarsazinyl, phenazinyl, phenothiazinyl, phenoxazinyl, and xanthenyl. In certain embodiments, the heteroaryl is optionally substituted with one or more substituents Q as described herein.

The terms “heteroarylene” and “heteroarenediyl” are used interchangeably herein in reference to a divalent monocyclic aromatic group or divalent polycyclic aromatic group that contains at least one aromatic ring, wherein at least one aromatic ring contains one or more heteroatoms in the ring, each of which is independently selected from O, S, and N. For a heteroarylene group containing a heteroaromatic ring and a nonaromatic heterocyclic ring, the heteroarylene group is not bonded to the rest of a molecule via its nonaromatic heterocyclic ring. Each ring of a heteroarylene group can contain one or two O atoms, one or two S atoms, and/or one to four N atoms, provided that the total number of heteroatoms in each ring is four or less and each ring contains at least one carbon atom. In certain embodiments, the heteroarylene has from 5 to 20, from 5 to 15, or from 5 to 10 ring atoms. Examples of monocyclic heteroarylene groups include, but are not limited to, furandiyl, imidazoldiyl, isothiazoldiyl, isoxazoldiyl, oxadiazoldiyl, oxazoldiyl, pyrazindiyl, pyrazoldiyl, pyridazindiyl, pyridindiyl, pyrimidindiyl, pyrroldiyl, thiadiazoldiyl, thiazoldiyl, thiendiyl, tetrazoldiyl, triazinediyl, and triazoldiyl. Examples of bicyclic heteroarylene groups include, but are not limited to, benzofurandiyl, benzimidazoldiyl, benzoisoxazoldiyl, benzopyrandiyl, benzothiadiazoldiyl, benzothiazoldiyl, benzothiendiyl, benzotriazoldiyl, benzoxazoldiyl, furopyridindiyl (including all isomeric forms, e.g., furo[2,3-b]pyridindiyl, furo[2,3-c]pyridindiyl, furo[3,2-b]pyridindiyl, furo[3,2-c]-pyridindiyl, furo[3,4-b]pyridindiyl, and furo[3,4-c]pyridindiyl), imidazopyridindiyl (including all isomeric forms, e.g., imidazo[1,2-a]pyridindiyl, imidazo[4,5-b]pyridindiyl, and imidazo[4,5-c]-pyridindiyl), imidazothiazoldiyl (including all isomeric forms, e.g., imidazo[2,1-b]thiazoldiyl and imidazo[4,5-d]thiazoldiyl), indazoldiyl, indolizindiyl, indoldiyl, isobenzofurandiyl, isobenzothiendiyl (i.e., benzo[c]thiendiyl), isoindoldiyl, isoquinolindiyl, naphthyridindiyl (including all isomeric forms, e.g., 1,5-naphthyridindiyl, 1,6-naphthyridindiyl, 1,7-naphthyridindiyl, and 1,8-naphthyridindiyl), oxazolopyridindiyl (including all isomeric forms, e.g., oxazolo[4,5-b]pyridindiyl, oxazolo[4,5-c]pyridindiyl, oxazolo[5,4-b]pyridindiyl, and oxazolo[5,4-c]pyridindiyl), phthalazindiyl, pteridindiyl, purindiyl, pyrrolopyridindiyl (including all isomeric forms, e.g., pyrrolo[2,3-b]pyridindiyl, pyrrolo[2,3-c]pyridindiyl, pyrrolo[3,2-b]-pyridindiyl, and pyrrolo[3,2-c]pyridindiyl), quinolindiyl, quinoxalindiyl, quinazolindiyl, thiadiazolopyrimidindiyl (including all isomeric forms, e.g., [1,2,5]thiadiazolo[3,4-d]-pyrimidindiyl and [1,2,3]thiadiazolo[4,5-d]pyrimidindiyl), and thienopyridindiyl (including all isomeric forms, e.g., thieno[2,3-b]pyridindiyl, thieno[2,3-c]pyridindiyl, thieno[3,2-b]pyridindiyl, and thieno[3,2-c]pyridindiyl). Examples of tricyclic heteroarylene groups include, but are not limited to, acridindiyl, benzindoldiyl, carbazoldiyl, dibenzofurandiyl, perimidindiyl, phenanthrolindiyl (including all isomeric forms, e.g., 1,5-phenanthrolindiyl, 1,6-phenanthrolindiyl, 1,7-phenanthrolindiyl, 1,9-phenanthrolindiyl, and 2,10-phenanthrolindiyl), phenanthridindiyl, phenarsazindiyl, phenazindiyl, phenothiazindiyl, phenoxazindiyl, and xanthendiyl. In certain embodiments, heteroarylene is optionally substituted with one or more substituents Q as described herein.

The term “heterocyclyl” or “heterocyclic” refers to a monovalent monocyclic non-aromatic ring system or monovalent polycyclic ring system that contains at least one non-aromatic ring, wherein one or more of the non-aromatic ring atoms are heteroatoms, each independently selected from O, S, and N; and the remaining ring atoms are carbon atoms. For a heterocyclyl group containing a heteroaromatic ring and a nonaromatic heterocyclic ring, the heterocyclyl group is not bonded to the rest of a molecule through the heteroaromatic ring. In certain embodiments, the heterocyclyl or heterocyclic group has from 3 to 20, from 3 to 15, from 3 to 10, from 3 to 8, from 4 to 7, or from 5 to 6 ring atoms. In certain embodiments, the heterocyclyl is a monocyclic, bicyclic, tricyclic, or tetracyclic ring system, which may be fused or bridged, and in which nitrogen or sulfur atoms may be optionally oxidized, nitrogen atoms may be optionally quaternized, and some rings may be partially or fully saturated, or aromatic. The heterocyclyl may be attached to the main structure at any heteroatom or carbon atom which results in the creation of a stable compound. Examples of heterocyclyls and heterocyclic groups include, but are not limited to, azepinyl, benzodioxanyl, benzodioxolyl, benzofuranonyl, chromanyl, decahydroisoquinolinyl, dihydrobenzofuranyl, dihydrobenzisothiazolyl, dihydro-benzisoxazinyl (including all isomeric forms, e.g., 1,4-dihydrobenzo[d][1,3]oxazinyl, 3,4-dihydrobenzo[c][1,2]-oxazinyl, and 3,4-dihydrobenzo[d][1,2]oxazinyl), dihydrobenzothienyl, dihydroisobenzofuranyl, dihydrobenzo[c]thienyl, dihydrofuryl, dihydroisoindolyl, dihydro-pyranyl, dihydropyrazolyl, dihydropyrazinyl, dihydropyridinyl, dihydropyrimidinyl, dihydro-pyrrolyl, dioxolanyl, 1,4-dithianyl, furanonyl, imidazolidinyl, imidazolinyl, indolinyl, isochromanyl, isoindolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, thiochromanyl, tetrahydroquinolinyl, and 1,3,5-trithianyl. In certain embodiments, the heterocyclyl is optionally substituted with one or more substituents Q as described herein.

The term “heterocyclylene” refers to a divalent monocyclic non-aromatic ring system or divalent polycyclic ring system that contains at least one non-aromatic ring, wherein one or more of the non-aromatic ring atoms are heteroatoms independently selected from O, S, and N; and the remaining ring atoms are carbon atoms. For a heterocyclylene group containing a heteroaromatic ring and a nonaromatic heterocyclic ring, the heterocyclylene group has at least one bond to the rest of a molecule via its nonaromatic heterocyclic ring. In certain embodiments, the heterocyclylene group has from 3 to 20, from 3 to 15, from 3 to 10, from 3 to 8, from 4 to 7, or from 5 to 6 ring atoms. In certain embodiments, the heterocyclylene is a monocyclic, bicyclic, tricyclic, or tetracyclic ring system, which may be fused or bridged, and in which nitrogen or sulfur atoms may be optionally oxidized, nitrogen atoms may be optionally quaternized, and some rings may be partially or fully saturated, or aromatic. The heterocyclylene may be attached to the main structure at any heteroatom or carbon atom which results in the creation of a stable compound. Examples of such heterocyclylene groups include, but are not limited to, azepindiyl, benzodioxandiyl, benzodioxoldiyl, benzofuranondiyl, chromandiyl, decahydroisoquinolindiyl, dihydrobenzofurandiyl, dihydrobenzisothiazoldiyl, dihydrobenzisoxazindiyl (including all isomeric forms, e.g., 1,4-dihydrobenzo[d][1,3]oxazindiyl, 3,4-dihydrobenzo[c][1,2]oxazindiyl, and 3,4-dihydrobenzo[d][1,2]oxazindiyl), dihydrobenzothiendiyl, dihydroisobenzofurandiyl, dihydrobenzo[c]thiendiyl, dihydrofurdiyl, dihydroisoindoldiyl, dihydropyrandiyl, dihydro-pyrazoldiyl, dihydropyrazindiyl, dihydropyridindiyl, dihydropyrimidindiyl, dihydropyrroldiyl, dioxolandiyl, 1,4-dithiandiyl, furanondiyl, imidazolidindiyl, imidazolindiyl, indolindiyl, isochromandiyl, isoindolindiyl, isothiazolidindiyl, isoxazolidindiyl, morpholindiyl, octahydro-indoldiyl, octahydroisoindoldiyl, oxazolidinondiyl, oxazolidindiyl, oxirandiyl, piperazindiyl, piperidindiyl, 4-piperidondiyl, pyrazolidindiyl, pyrazolindiyl, pyrrolidindiyl, pyrrolindiyl, quinuclidindiyl, tetrahydrofurdiyl, tetrahydroisoquinolindiyl, tetrahydropyrandiyl, tetrahydro-thiendiyl, thiamorpholindiyl, thiazolidindiyl, thiochromandiyl, tetrahydroquinolindiyl, and 1,3,5-trithiandiyl. In certain embodiments, the heterocyclylene is optionally substituted with one or more substituents Q as described herein.

The term “halogen,” “halide,” or “halo” refers to fluoro, chloro, bromo, and/or iodo.

The term “optionally substituted” is intended to mean that a group or substituent, such as an alkyl, alkylene, heteroalkyl, heteroalkylene, alkenyl, alkenylene, heteroalkenylene, alkynyl, alkynylene, heteroalkynylene, cycloalkyl, cycloalkylene, aryl, arylene, aralkyl, aralkylene, heteroaryl, heteroarylene, heterocyclyl, or heterocyclylene group, may be substituted with one or more, in one embodiment, one, two, three, or four, substituents Q, each of which is independently selected from, e.g., (a) deuterium (-D), cyano (—CN), halo, imino (═NH), nitro (—NO₂), and oxo (═O); (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{b})OR^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{1a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl optionally substituted with one or more, in one embodiment, one, two, three, or four, substituents Q^{a}. As used herein, all groups that can be substituted are “optionally substituted.”

In one embodiment, each Q^{a} is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OP(O)(OR^{f})OR^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, “optically active” and “enantiomerically active” refer to a collection of molecules, which has an enantiomeric excess of no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, an optically active compound comprises about 95% or more of one enantiomer and about 5% or less of the other enantiomer based on the total weight of the enantiomeric mixture in question. In certain embodiments, an optically active compound comprises about 98% or more of one enantiomer and about 2% or less of the other enantiomer based on the total weight of the enantiomeric mixture in question. In certain embodiments, an optically active compound comprises about 99% or more of one enantiomer and about 1% or less of the other enantiomer based on the total weight of the enantiomeric mixture in question.

In describing an optically active compound, the prefixes R and S are used to denote the absolute configuration of the compound about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the compound, R and S.

The term “isotopically enriched” refers to a compound that contains an unnatural proportion of an isotope at one or more of the atoms that constitute such a compound. In certain embodiments, an isotopically enriched compound contains unnatural proportions of one or more isotopes, including, but not limited to, hydrogen (¹H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²¹I), iodine-127 (¹²⁷I), iodine-129 (¹²⁹I), and iodine-131 (¹³¹I). In certain embodiments, an isotopically enriched compound is in a stable form, that is, non-radioactive. In certain embodiments, an isotopically enriched compound contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an isotopically enriched compound is in an unstable form, that is, radioactive. In certain embodiments, an isotopically enriched compound contains unnatural proportions of one or more isotopes, including, but not limited to, tritium (3H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I), iodine-125 (¹²⁵I), iodine-129 (¹²⁹I), and iodine-131 (¹³¹I). It will be understood that, in a compound as provided herein, any hydrogen can be ²H, as example, or any carbon can be ¹³C, as example, or any nitrogen can be ¹⁵N, as example, or any oxygen can be ¹⁸O, as example, where feasible according to the judgment of one of ordinary skill in the art.

The term “isotopic enrichment” refers to the percentage of incorporation of a less prevalent isotope (e.g., D for deuterium or hydrogen-2) of an element at a given position in a molecule in the place of a more prevalent isotope (e.g., ¹H for protium or hydrogen-1) of the element. As used herein, when an atom at a particular position in a molecule is designated as a particular less prevalent isotope, it is understood that the abundance of that isotope at that position is substantially greater than its natural abundance.

The term “isotopic enrichment factor” refers the ratio between the isotopic abundance in an isotopically enriched compound and the natural abundance of a specific isotope.

The term “hydrogen” or the symbol “H” refers to the composition of naturally occurring hydrogen isotopes, which include protium (¹H), deuterium (²H or D), and tritium (³H), in their natural abundances. Protium is the most common hydrogen isotope having a natural abundance of more than 99.98%. Deuterium is a less prevalent hydrogen isotope having a natural abundance of about 0.0156%.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156% on average, deuterium enrichment at any position in a compound synthesized using non-enriched starting materials is about 0.0156% on average. As used herein, when a particular position in an isotopically enriched compound is designated as having deuterium, it is understood that the abundance of deuterium at that position in the compound is substantially greater than its natural abundance (0.0156%).

The term “carbon” or the symbol “C” refers to the composition of naturally occurring carbon isotopes, which include carbon-12 (¹²C) and carbon-13 (¹³C) in their natural abundances. Carbon-12 is the most common carbon isotope having a natural abundance of more than 98.89%. Carbon-13 is a less prevalent carbon isotope having a natural abundance of about 1.11%.

The term “carbon-13 enrichment” or “¹³C enrichment” refers to the percentage of incorporation of carbon-13 at a given position in a molecule in the place of carbon. For example, carbon-13 enrichment of 10% at a given position means that 10% of molecules in a given sample contain carbon-13 at the specified position. Because the naturally occurring distribution of carbon-13 is about 1.11% on average, carbon-13 enrichment at any position in a compound synthesized using non-enriched starting materials is about 1.11% on average. As used herein, when a particular position in an isotopically enriched compound is designated as having carbon-13, it is understood that the abundance of carbon-13 at that position in the compound is substantially greater than its natural abundance (1.11%).

The terms “substantially pure” and “substantially homogeneous” mean, when referred to a substance, sufficiently homogeneous to appear free of readily detectable impurities as determined by a standard analytical method used by one of ordinary skill in the art, including, but not limited to, thin layer chromatography (TLC), gel electrophoresis, high performance liquid chromatography (HPLC), gas chromatography (GC), nuclear magnetic resonance (NMR), and mass spectrometry (MS); or sufficiently pure such that further purification would not detectably alter the physical, chemical, biological, and/or pharmacological properties, such as enzymatic and biological activities, of the substance. In certain embodiments, “substantially pure” or “substantially homogeneous” refers to a collection of molecules, wherein at least about 95%, at least about 96%, at least about 97%, at least about 98%, at least about 99%, or at least about 99.5% by weight of the molecules are a single compound, including a single enantiomer, a racemic mixture, or a mixture of enantiomers, as determined by standard analytical methods. As used herein, when an atom at a particular position in an isotopically enriched molecule is designated as a particular less prevalent isotope, a molecule that contains other than the designated isotope at the specified position is an impurity with respect to the isotopically enriched compound. Thus, for a deuterated compound that has an atom at a particular position designated as deuterium, a compound that contains a protium at the same position is an impurity.

The term “solvate” refers to a complex or aggregate formed by one or more molecules of a solute, e.g., a compound provided herein, and one or more molecules of a solvent, which are present in a stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

For a divalent group described herein, no orientation is implied by the direction in which the divalent group is presented. For example, unless a particular orientation is specified, the formula —C(O)NH— represents both —C(O)NH— and —NHC(O)—.

The phrase “an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof” has the same meaning as the phrase “(i) an enantiomer, a mixture of enantiomers, a diastereomer, a mixture of two or more diastereomers, a tautomer, a mixture of two or more tautomers, or an isotopic variant of the compound referenced therein; (ii) a pharmaceutically acceptable salt, solvate, hydrate, or prodrug of the compound referenced therein; or (iii) a pharmaceutically acceptable salt, solvate, hydrate, or prodrug of an enantiomer, a mixture of enantiomers, a diastereomer, a mixture of two or more diastereomers, a tautomer, a mixture of two or more tautomers, or an isotopic variant of the compound referenced therein.”

### Compounds

In one embodiment, provided herein is a compound of Formula (A):

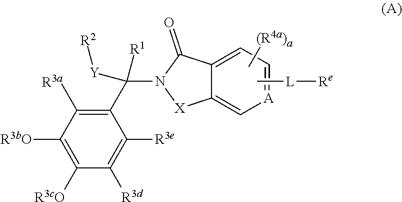

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
A is C(R⁴) or N;
L is a linker;
X is —CH₂— or —C(O)—;
Y is C₁₋₆ alkylene or C₃₋₁₀ cycloalkylene;
R¹ is hydrogen or deuterium;
R² is cyano or —S(O₂)R^{2a};
R⁴ is hydrogen or R^{4a};
R^{2a} is (i) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (ii) —OR^{1a} or —NR^{1b}R^{1c};
R^{3a}, R^{3d}, and R^{3e} are each independently (i) hydrogen, deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{3b} and R^{3c} are each independently C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
each R^{4a} is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})R^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{e} is

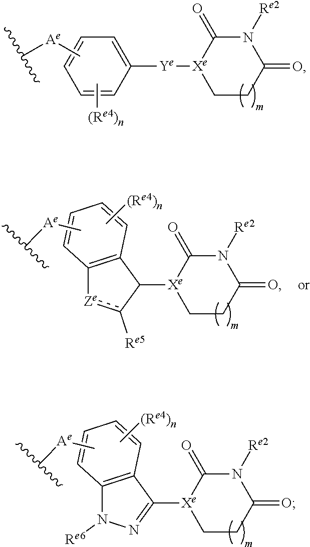

wherein:
   each A^{e} is independently (i) C₁₋₆ alkylene, C₂₋₆ alkenylene, C₂₋₆ alkynylene, C₃₋₁₀ cycloalkylene, C₆₋₁₄ arylene, heteroarylene, or heterocyclylene;
or (ii) —O— or —NHCH₂CH₂—;
   X^{e} is C(R^{e1}) or N;
   Y^{e} is a bond, C₁₋₆ alkylene, —O—, —S—, —S(O)—, —S(O₂)—, or —N(R^{e3})—;
   Z^{e} is —CH═, —CH₂—, —N═, or —N(R^{e6})—;
   R^{c1} is hydrogen, deuterium, halo, or C₁₋₆ alkyl;
   each R^{e2}, R^{e3}, and R^{e6} is independently hydrogen or C₁₋₆ alkyl;
   each R^{e4} is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —C(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
   R^{e5} is hydrogen or oxo;
   each m is independently an integer of 0, 1, or 2; and
   each n is independently an integer of 0, 1, 2, 3, or 4;
each R^{1a}, R^{1b}, R^{1c}, and R^{1d} is independently hydrogen, deuterium, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; and
a is an integer of 0, 1, 2, 3, or 4;
wherein each alkyl, alkylene, heteroalkyl, alkenyl, alkenylene, alkynyl, alkynylene, cycloalkyl, cycloalkylene, aryl, arylene, aralkyl, heteroaryl, heteroarylene, heterocyclyl, and heterocyclylene is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{a})OR^{d}, —OS(O)R^{a}, —OS(O)₂R^{a}, —OS(O)NR^{1b}R^{1c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OP(O)(OR^{f})OR^{g}, —OS(O)R^{e}, —OS(O)₂R^{e}, —OSO)NR^{f}R^{g}, —OS(O)₂NR^{f}R^{g}, —NR^{f}R^{g}, —NR^{e}C(O)R^{h}, —NR^{e}C(O)OR, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{g}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, A is C(R⁴), wherein R⁴ is as defined herein. In certain embodiments, A is N.

In another embodiment, provided herein is a compound of Formula (I):

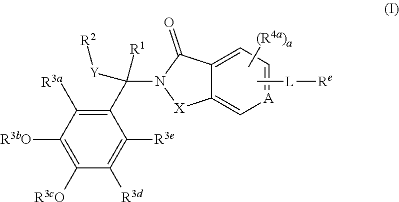

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
L is a linker;
X is —CH₂— or —C(O)—;
Y is C₁₋₆ alkylene or C₃₋₁₀ cycloalkylene;
R¹ is hydrogen or deuterium;
R² is cyano or —S(O₂)R^{2a};
R^{2a} is (i) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (ii) —OR^{1a} or —NR^{1b}R^{1c};
R^{3a}, R^{3d}, and R^{3e} are each independently (i) hydrogen, deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —R^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{3b} and R^{3c} are each independently C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
each R^{4a} is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(Nd)NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{e} is

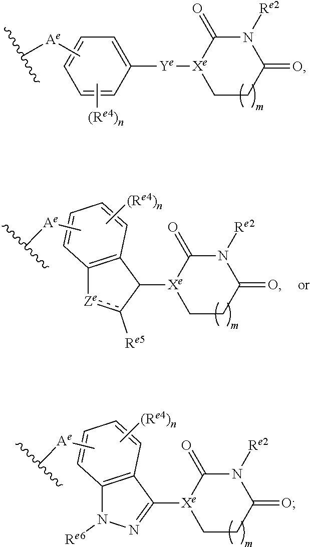

wherein:
   each A^{e} is independently (i) C₁₋₆ alkylene, C₂₋₆ alkenylene, C₂₋₆ alkynylene, C₃₋₁₀ cycloalkylene, C₆₋₁₄ arylene, heteroarylene, or heterocyclylene;
or (ii) —O— or —NHCH₂CH₂—;
   X^{e} is C(R^{e1}) or N;
   Y^{e} is a bond, C₁₋₆ alkylene, —O—, —S—, —S(O)—, —S(O₂)—, or —N(R^{e3})—;
   Z^{e} is —CH═, —CH₂—, —N═, or —N(R^{e6})—;
   R^{e1} is hydrogen, deuterium, halo, or C₁₋₆ alkyl;
   each R^{e2}, R^{e3}, and R^{e6} is independently hydrogen or C₁₋₆ alkyl;
   each R^{e4} is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
   R^{e5} is hydrogen or oxo;
   each m is independently an integer of 0, 1, or 2; and
   each n is independently an integer of 0, 1, 2, 3, or 4;
each R^{1a}, R^{1b}, R^{1c}, and R^{1d} is independently hydrogen, deuterium, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; and
a is an integer of 0, 1, 2, 3, or 4;
wherein each alkyl, alkylene, heteroalkyl, alkenyl, alkenylene, alkynyl, alkynylene, cycloalkyl, cycloalkylene, aryl, arylene, aralkyl, heteroaryl, heteroarylene, heterocyclyl, and heterocyclylene is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{a})OR^{d}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OP(O)(OR^{f})OR^{g}, —OS(O)R^{c}, —OS(O)₂R^{c}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})R^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, a compound provided herein is not any one of 6-[4-({[4-(2,6-dioxopiperidin-3-yl)phenyl]carbamoyl}methyl)piperazin-1-yl]-N-{2-[(1S)-1-(3-ethoxy-4-methoxyphenyl)-2-methanesulfonylethyl]-1,3-dioxo-2,3-dihydro-1H-isoindol-4-yl}hexanamide; 8-[4-({[4-(2,6-dioxopiperidin-3-yl)phenyl]carbamoyl}methyl)piperazin-1-yl]-N-{2-[(1S)-1-(3-ethoxy-4-methoxyphenyl)-2-methanesulfonylethyl]-1,3-dioxo-2,3-dihydro-1H-isoindol-4-yl}-octanamide; 8-[4-({[3-(2,6-dioxopiperidin-3-yl)phenyl]carbamoyl}methyl)piperazin-1-yl]-N-{2-[(1S)-1-(3-ethoxy-4-methoxyphenyl)-2-methanesulfonylethyl]-1,3-dioxo-2,3-dihydro-1H-isoindol-4-yl}octanamide; 10-[4-({[4-(2,6-dioxopiperidin-3-yl)phenyl]carbamoyl}methyl)-piperazin-1-yl]-N-{2-[(1S)-1-(3-ethoxy-4-methoxyphenyl)-2-methanesulfonylethyl]-1,3-dioxo-2,3-dihydro-1H-isoindol-4-yl}decanamide; 6-[4-({[3-(2,6-dioxopiperidin-3-yl)phenyl]-carbamoyl}methyl)piperazin-1-yl]-N-{2-[(1S)-1-(3-ethoxy-4-methoxyphenyl)-2-methanesulfonylethyl]-1,3-dioxo-2,3-dihydro-1H-isoindol-4-yl}hexanamide; 10-[4-({[3-(2,6-dioxo-piperidin-3-yl)phenyl]carbamoyl}methyl)piperazin-1-yl]-N-{2-[(1S)-1-(3-ethoxy-4-methoxy-phenyl)-2-methanesulfonylethyl]-1,3-dioxo-2,3-dihydro-1H-isoindol-4-yl}decanamide; N-{6-[12-({3-[(2,6-dioxopiperidin-3-yl)amino]phenyl}amino)dodecyl]-2-[(1S)-1-(3-ethoxy-4-methoxyphenyl)-2-methanesulfonylethyl]-1,3-dioxo-2,3-dihydro-1H-isoindol-4-yl}acetamide; 4-amino-6-[12-({3-[(2,6-dioxopiperidin-3-yl)amino]phenyl}amino)dodecyl]-2-[(1S)-1-(3-ethoxy-4-methoxyphenyl)-2-methanesulfonylethyl]-2,3-dihydro-1H-isoindole-1,3-dione; 9-({3-[(2,6-dioxopiperidin-3-yl)amino]phenyl}amino)-N-(2-[(1S)-1-(3-ethoxy-4-methoxyphenyl)-2-methanesulfonylethyl]-1,3-dioxo-2,3-dihydro-1H-isoindol-4-yl)nonanamide; and 6-{4-[({3-[(2,6-dioxopiperidin-3-yl)amino]phenyl}carbamoyl)methyl]piperazin-1-yl}-N-{2-[(1S)-1-(3-ethoxy-4-methoxyphenyl)-2-methanesulfonylethyl]-1,3-dioxo-2,3-dihydro-1H-isoindol-4-yl}-hexanamide.

In certain embodiments, a compound provided herein is not any one of 8-[4-({[3-(2,6-dioxopiperidin-3-yl)-1-methyl-1H-indazol-6-yl]carbamoyl}-methyl)piperazin-1-yl]-N-{2-[(1S)-1-(3-ethoxy-4-methoxyphenyl)-2-methanesulfonylethyl]-1,3-dioxo-2,3-dihydro-1H-isoindol-4-yl}octanamide; 6-[4-({[3-(2,6-dioxopiperidin-3-yl)-1-methyl-1H-indazol-6-yl]-carbamoyl}-methyl)piperazin-1-yl]-N-{2-[(1S)-1-(3-ethoxy-4-methoxyphenyl)-2-methanesulfonylethyl]-1,3-dioxo-2,3-dihydro-1H-isoindol-4-yl}hexanamide; 10-[4-({[3-(2,6-dioxo-piperidin-3-yl)-1-methyl-1H-indazol-6-yl]carbamoyl}-methyl)piperazin-1-yl]-N-{2-[(1S)-1-(3-ethoxy-4-methoxyphenyl)-2-methanesulfonylethyl]-1,3-dioxo-2,3-dihydro-1H-isoindol-4-yl}decanamide; 8-[4-({[3-(2,6-dioxopiperidin-3-yl)-1-methyl-1H-indazol-7-yl]carbamoyl}-methyl)piperazin-1-yl]-N-{2-[(1S)-1-(3-ethoxy-4-methoxyphenyl)-2-methanesulfonylethyl]-1,3-dioxo-2,3-dihydro-1H-isoindol-4-yl}octanamide; 10-[4-({[3-(2,6-dioxopiperidin-3-yl)-1-methyl-1H-indazol-7-yl]carbamoyl}-methyl)piperazin-1-yl]-N-{2-[(1S)-1-(3-ethoxy-4-methoxyphenyl)-2-methanesulfonylethyl]-1,3-dioxo-2,3-dihydro-1H-isoindol-4-yl}decanamide; and 6-[4-({[3-(2,6-dioxopiperidin-3-yl)-1-methyl-1H-indazol-7-yl]carbamoyl}-methyl)piperazin-1-yl]-N-{2-[(1S)-1-(3-ethoxy-4-methoxyphenyl)-2-methanesulfonylethyl]-1,3-dioxo-2,3-dihydro-1H-isoindol-4-yl}hexanamide.

In yet another embodiment, provided herein is a compound of Formula (IA):

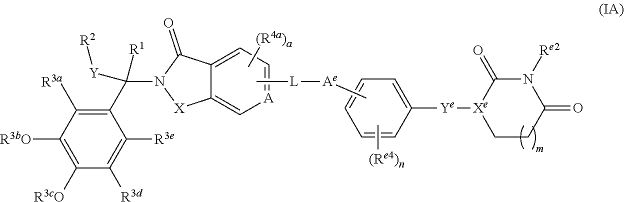

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, A^{e}, L, X, X^{e}, Y, Y^{e}, a, m, and n are each as defined herein.

In yet another embodiment, provided herein is a compound of Formula (IB):

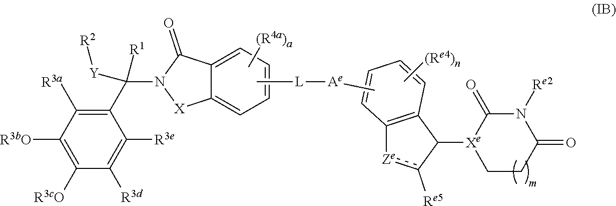

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3s}, R^{3b}, R^{3c}, R^{3d}, R^{3e}, R^{4a}, R^{e2}, R^{e4}, R^{e5}, A^{e}, L, X, Y, Z^{e}, a, m, and n are each as defined herein.

In yet another embodiment, provided herein is a compound of Formula (IC):

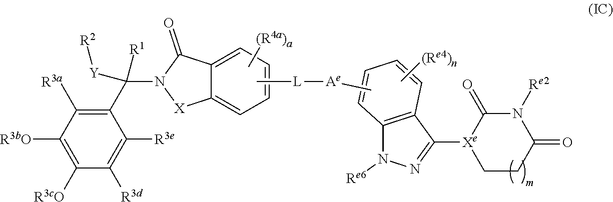

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, R^{e6}, A^{e}, L, X, Y, X^{e}, a, m, and n are each as defined herein.

In yet another embodiment, provided herein is a compound of Formula (ID):

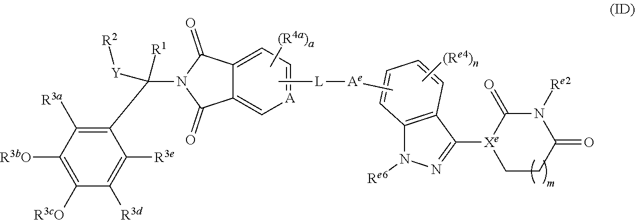

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, R^{e6}, AA^{e}, L, Y, X^{e}, a, m, and n are each as defined herein.

In certain embodiments, Y is C₁₋₆ alkylene, optionally substituted with one or more substituents Q. In certain embodiments, Y is methylene, optionally substituted with one or more substituents Q. In certain embodiments, Y is —CH₂— or —CD₂-. In certain embodiments, Y is C₃₋₁₀ cycloalkylene, optionally substituted with one or more substituents Q. In certain embodiments, Y is cyclopropanediyl. In certain embodiments, Y is cyclopropane-1,1-diyl.

In certain embodiments, Y^{e} is a bond. In certain embodiments, Y^{e} is C₁₋₆ alkylene, optionally substituted with one or more substituents Q. In certain embodiments, Y^{e} is —O—. In certain embodiments, Y^{e} is —S—. In certain embodiments, Y^{e} is —S(O)—. In certain embodiments, Y^{e} is —S(O₂)—. In certain embodiments, Y^{e} is —N(R^{e3})—, wherein R^{e3} is as defined herein. In certain embodiments, Y^{e} is —N(H)—.

In one embodiment, provided herein is a compound of Formula (IIA):

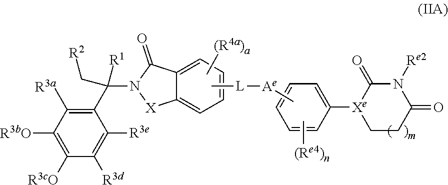

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, A^{e}, L, X, X^{e}, a, m, and n are each as defined herein.

In certain embodiments, X^{e} is C(R^{e1}), wherein R^{e1} is as defined herein. In certain embodiments, X^{e} is C(H). In certain embodiments, X^{e} is N.

In another embodiment, provided herein is a compound of Formula (IIIA):

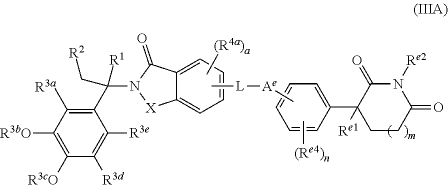

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof, wherein R¹, R², R^{3a}, R^{3b}, R^{3c}, R^{3d}, R^{3c}, R^{4a}, R^{c1}, R^{c2}, R^{c4}, A^{c}, L, X, a, m, and n are each as defined herein.

In one embodiment, provided herein is a compound of Formula (IVA):

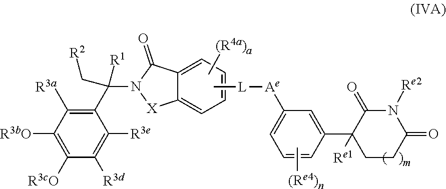

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e1}, R^{e2}, R^{e4}, A^{e}, L, X, a, m, and n are each as defined herein.

In another embodiment, provided herein is a compound of Formula (VA):

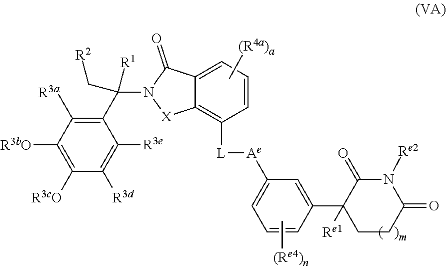

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e1}, R^{e2}, R^{e4}, A^{e}, L, X, a, m, and n are each as defined herein.

In yet another embodiment, provided herein is a compound of Formula (VIA):

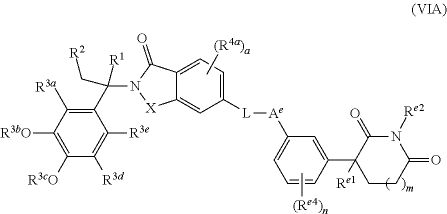

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e1}, R^{e2}, R^{e4}, A^{e}, L, X, a, m, and n are each as defined herein.

In still another embodiment, provided herein is a compound of Formula (VIIA):

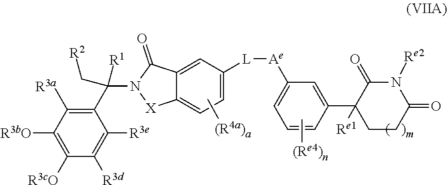

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e1}, R^{e2}, R^{e4}, A^{e}, L, X, a, m, and n are each as defined herein.

In one embodiment, provided herein is a compound of Formula (VIIIA):

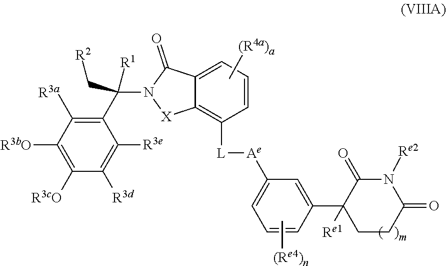

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e1}, R^{e2}, R^{e4}, A^{e}, L, X, a, m, and n are each as defined herein.

In another embodiment, provided herein is a compound of Formula (IXA):

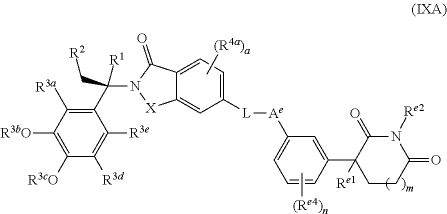

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e1}, R^{e2}, R^{e4}, A^{e}, L, X, a, m, and n are each as defined herein.

In yet another embodiment, provided herein is a compound of Formula (XA):

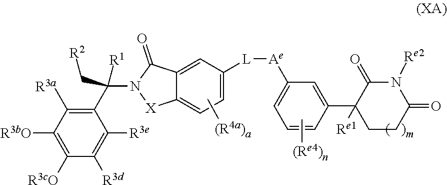

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e1}, R^{e2}, R^{e4}, A^{e}, L, X, a, m, and n are each as defined herein.

In one embodiment, provided herein is a compound of Formula (XIA):

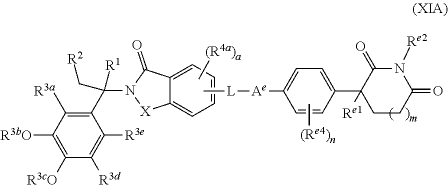

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e1}, R^{e2}, R^{e4}, A^{e}, L, X, a, m, and n are each as defined herein.

In another embodiment, provided herein is a compound of Formula (XIIA):

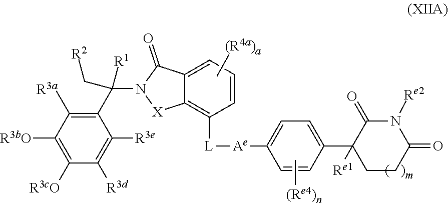

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e1}, R^{e2}, R^{e4}, A^{e}, L, X, a, m, and n are each as defined herein.

In yet another embodiment, provided herein is a compound of Formula (XIIIA):

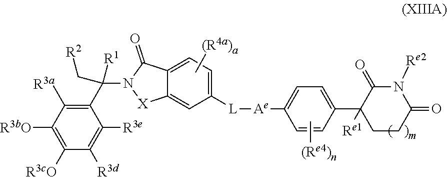

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e1}, R^{e2}, R^{e4}, A^{e}, L, X, a, m, and n are each as defined herein.

In still another embodiment, provided herein is a compound of Formula (XIVA):

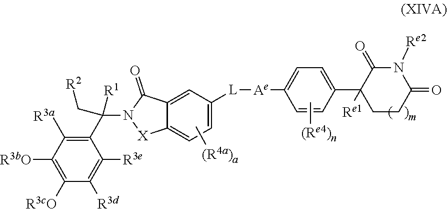

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e1}, R^{e2}, R^{e4}, A^{e}, L, X, a, m, and n are each as defined herein.

In one embodiment, provided herein is a compound of Formula (XVA):

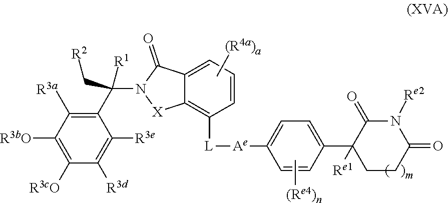

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e1}, R^{e2}, R^{e4}, A^{e}, L, X, a, m, and n are each as defined herein.

In another embodiment, provided herein is a compound of Formula (XVIA):

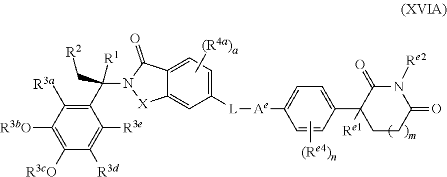

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e1}, R^{e2}, R^{e4}, A^{e}, L, X, a, m, and n are each as defined herein.

In yet another embodiment, provided herein is a compound of Formula (XVIIA):

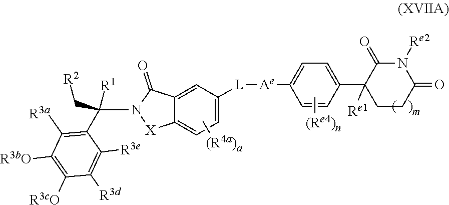

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e1}, R^{e2}, R^{e4}, A^{e}, L, X, a, m, and n are each as defined herein.

In one embodiment, provided herein is a compound of Formula (XVIIIA):

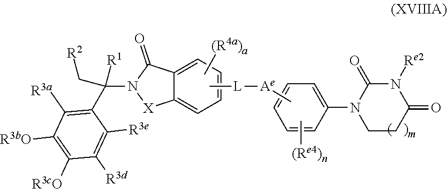

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, A^{e}, L, X, a, m, and n are each as defined herein.

In one embodiment, provided herein is a compound of Formula (XIXA):

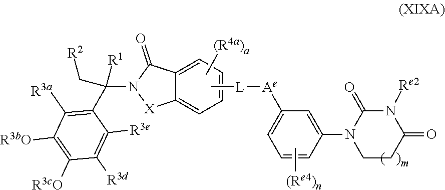

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, A^{e}, L, X, a, m, and n are each as defined herein.

In another embodiment, provided herein is a compound of Formula (XXA):

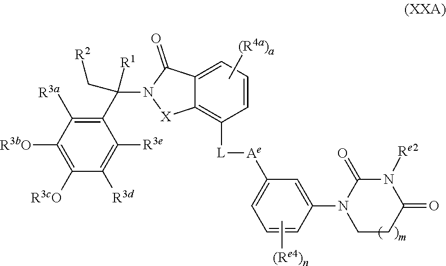

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e2}, R^{e4}, A^{e}, L, X, a, m, and n are each as defined herein.

In yet another embodiment, provided herein is a compound of Formula (XXIA):

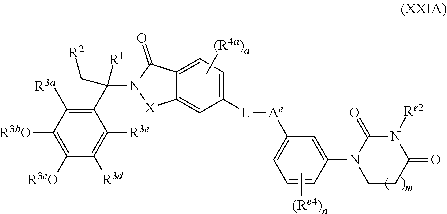

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e2}, R^{e4}, A^{e}, L, X, a, m, and n are each as defined herein.

In still another embodiment, provided herein is a compound of Formula (XXIIA):

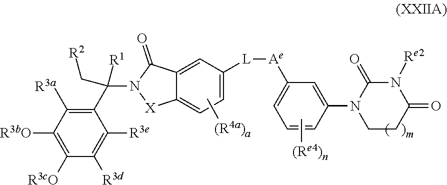

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e2}, R^{e4}, A^{e}, L, X, a, m, and n are each as defined herein.

In one embodiment, provided herein is a compound of Formula (XXIIIA):

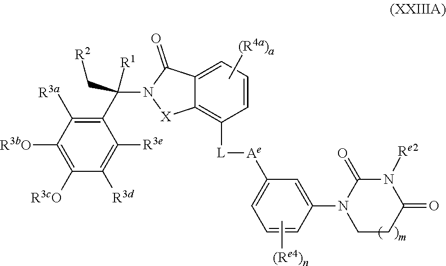

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof, wherein R¹, R², R^{3a}, R^{3b}, R^{3c}, R^{3d}, R^{3c}, R^{4a}, R^{c2}, R^{c4}, A^{c}, L, X, a, m, and n are each as defined herein.

In another embodiment, provided herein is a compound of Formula (XXIVA):

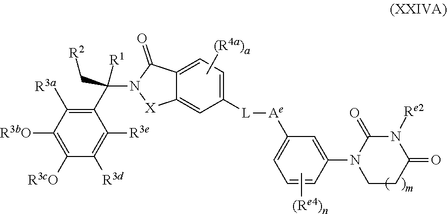

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, A^{e}, L, X, a, m, and n are each as defined herein.

In yet another embodiment, provided herein is a compound of Formula (XXVA):

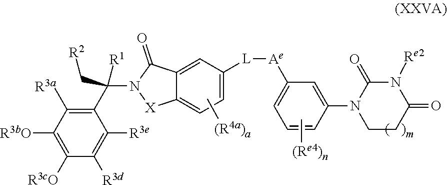

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, A^{e}, L, X, a, m, and n are each as defined herein.

In one embodiment, provided herein is a compound of Formula (XXVIA):

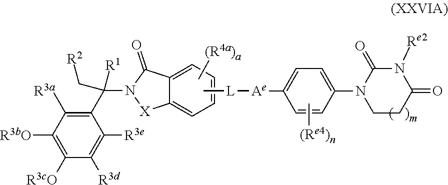

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e2}, R^{e4}, A^{e}, L, X, a, m, and n are each as defined herein.

In another embodiment, provided herein is a compound of Formula (XXVIIA):

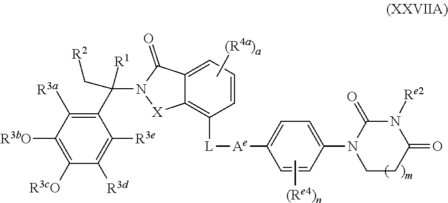

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e2}, R^{e4}, A^{e}, L, X, a, m, and n are each as defined herein.

In yet another embodiment, provided herein is a compound of Formula (XXVIIIA):

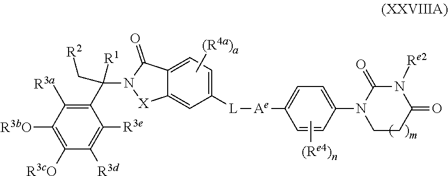

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, A^{e}, L, X, a, m, and n are each as defined herein.

In still another embodiment, provided herein is a compound of Formula (XXIXA):

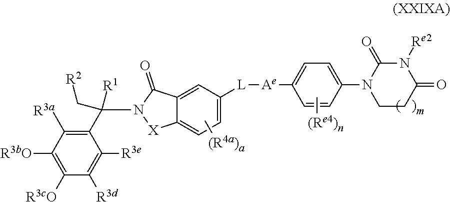

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, A^{e}, L, X, a, m, and n are each as defined herein.

In one embodiment, provided herein is a compound of Formula (XXXA):

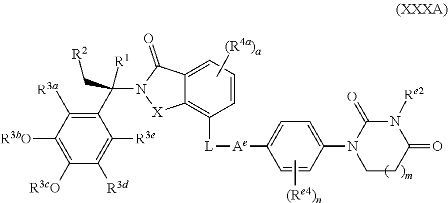

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, A^{e}, L, X, a, m, and n are each as defined herein.

In another embodiment, provided herein is a compound of Formula (XXXIA):

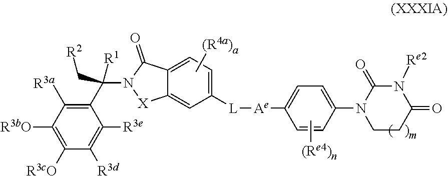

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e2}, R^{e4}, A^{e}, L, X, a, m, and n are each as defined herein.

In yet another embodiment, provided herein is a compound of Formula (XXXIIA):

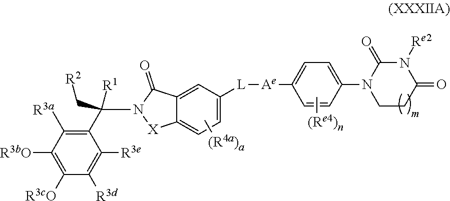

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e2}, R^{e4}, A^{e}, L, X, a, m, and n are each as defined herein.

In one embodiment, provided herein is a compound of Formula (XXXIIIA):

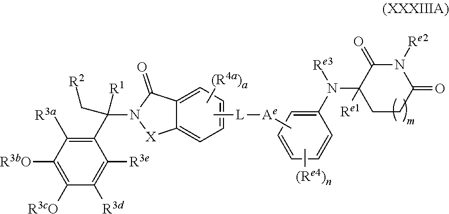

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e1}, R^{e2}, R^{e3}, R^{e4}, A^{e}, L, X, a, m, and n are each as defined herein.

In one embodiment, provided herein is a compound of Formula (XXXIVA):

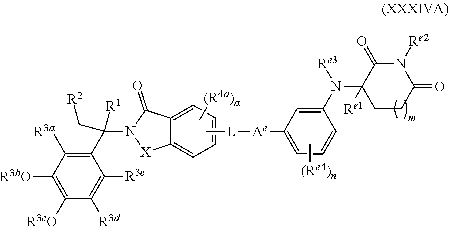

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof, wherein R¹, R², R^{3a}, R^{3b}, R^{3c}, R^{3d}, R^{3e}, R^{4a}, R^{e1}, R^{e2}, R^{e3}, R^{e4}, A^{e}, L, X, a, m, and n are each as defined herein.

In another embodiment, provided herein is a compound of Formula (XXXVA):

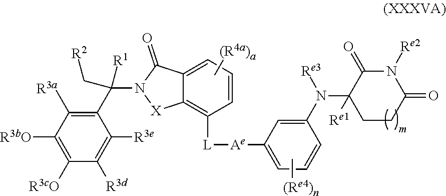

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e1}, R^{e2}, R^{e3}, R^{e4}, A^{e}, L, X, a, m, and n are each as defined herein.

In yet another embodiment, provided herein is a compound of Formula (XXXVIA):

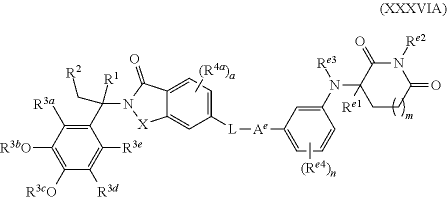

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e1}, R^{e2}, R^{e3}, R^{e4}, A^{e}, L, X, a, m, and n are each as defined herein.

In still another embodiment, provided herein is a compound of Formula (XXXVIIA):

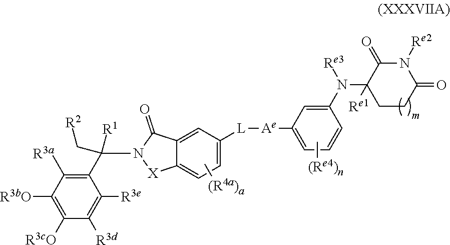

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e1}, R^{e2}, R^{e3}, R^{e4}, A^{e}, L, X, a, m, and n are each as defined herein.

In one embodiment, provided herein is a compound of Formula (XXXVIIIA):

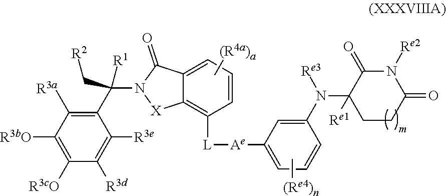

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e1}, R^{e2}, R^{e3}, R^{e4}, A^{e}, L, X, a, m, and n are each as defined herein.

In another embodiment, provided herein is a compound of Formula (XXXIXA):

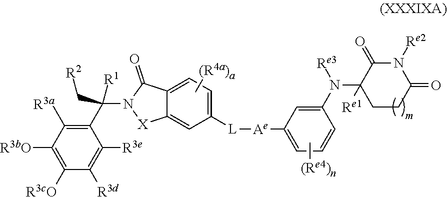

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e1}, R^{e2}, R^{e3}, R^{e4}, A^{e}, L, X, a, m, and n are each as defined herein.

In yet another embodiment, provided herein is a compound of Formula (XLA):

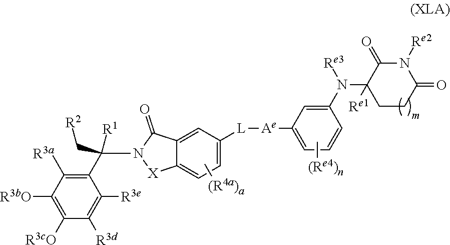

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e1}, R^{e2}, R^{e3}, R^{e4}, A^{e}, L, X, a, m, and n are each as defined herein.

In one embodiment, provided herein is a compound of Formula (XLIA):

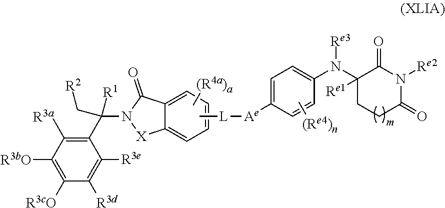

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e1}, R^{e2}, R^{e3}, R^{e4}, A^{e}, L, X, a, m, and n are each as defined herein.

In another embodiment, provided herein is a compound of Formula (XLIIA):

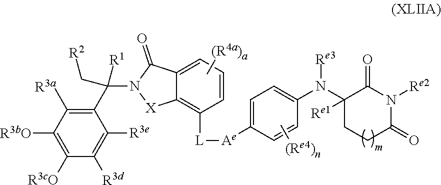

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e1}, R^{e2}, R^{e3}, R^{e4}, A^{e}, L, X, a, m, and n are each as defined herein.

In yet another embodiment, provided herein is a compound of Formula (XLIIA):

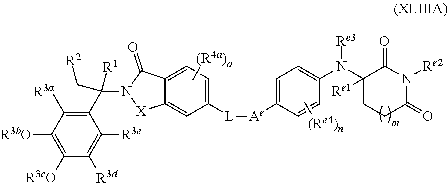

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e1}, R^{e2}, R^{e3}, R^{e4}, A^{e}, L, X, a, m, and n are each as defined herein.

In still another embodiment, provided herein is a compound of Formula (XLIVA):

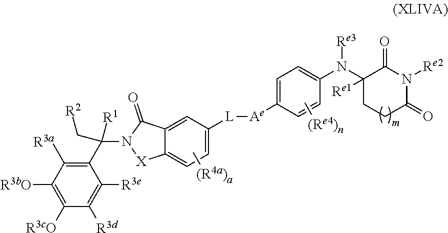

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e1}, R^{e2}, R^{e3}, R^{e4}, A^{e}, L, X, a, m, and n are each as defined herein.

In one embodiment, provided herein is a compound of Formula (XLVA):

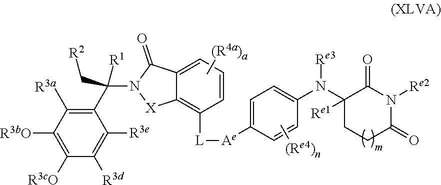

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e1}, R^{e2}, R^{e3}, R^{e4}, A^{e}, L, X, a, m, and n are each as defined herein.

In another embodiment, provided herein is a compound of Formula (XLVIA):

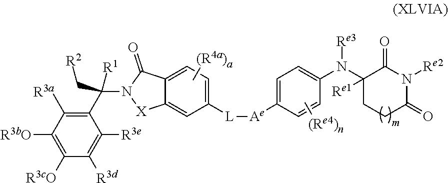

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e1}, R^{e2}, R^{e3}, R^{e4}, A^{e}, L, X, a, m, and n are each as defined herein.

In yet another embodiment, provided herein is a compound of Formula (XLVIIA):

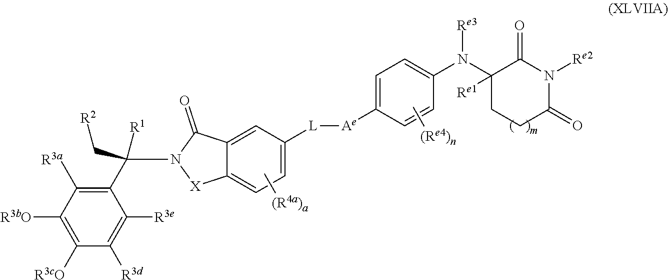

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e1}, R^{e2}, R^{e3}, R^{e4}, A^{e}, L, X, a, m, and n are each as defined herein.

In certain embodiments, Z^{e} is —CH═. In certain embodiments, Z^{e} is —CH₂—. In certain embodiments, Z^{e} is —N═. In certain embodiments, Z^{e} is —N(R^{e6})—, wherein R^{e6} is as defined herein. In certain embodiments, Z^{e} is —N(CH₃)—.

In one embodiment, provided herein is a compound of Formula (IIB):

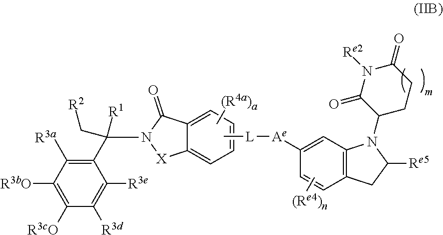

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, R^{e5}, A^{e}, L, X, a, m, and n are each as defined herein.

In another embodiment, provided herein is a compound of Formula (IIIB):

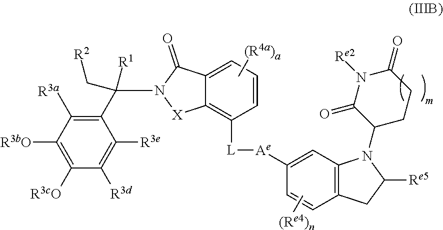

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, R^{e5}, A^{e}, L, X, a, m, and n are each as defined herein.

In yet another embodiment, provided herein is a compound of Formula (IVB):

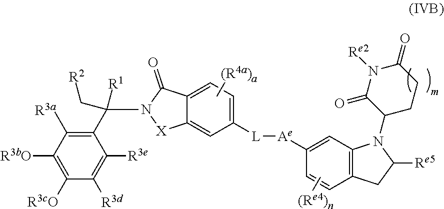

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, R^{e5}, A^{e}, L, X, a, m, and n are each as defined herein.

In still another embodiment, provided herein is a compound of Formula (VB):

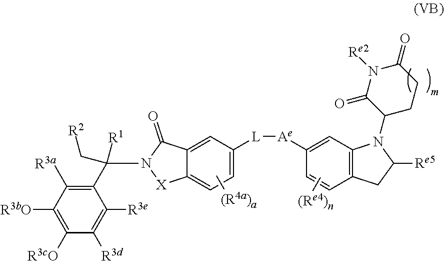

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, R^{e5}, A^{e}, L, X, a, m, and n are each as defined herein.

In one embodiment, provided herein is a compound of Formula (VIB):

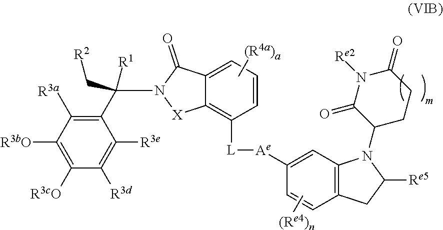

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, R^{e5}, A^{e}, L, X, a, m, and n are each as defined herein.

In another embodiment, provided herein is a compound of Formula (VIIB):

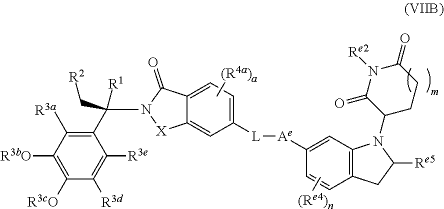

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, A^{e}, L, X, a, m, and n are each as defined herein.

In yet another embodiment, provided herein is a compound of Formula (VIIB):

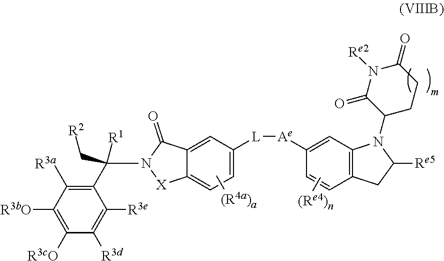

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e2}, R^{e4}, R^{e5}, A^{e}, L, X, a, m, and n are each as defined herein.

In one embodiment, provided herein is a compound of Formula (IXB):

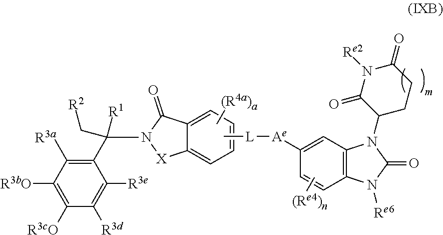

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e2}, R^{e4}, R^{e6}, A^{e}, L, X, a, m, and n are each as defined herein.

In another embodiment, provided herein is a compound of Formula (XB):

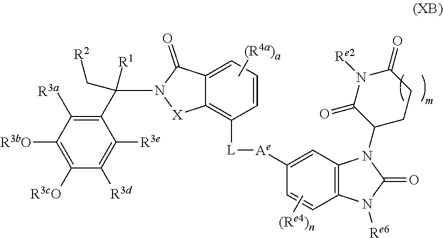

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, R^{e6}, A^{e}, L, X, a, m, and n are each as defined herein.

In yet another embodiment, provided herein is a compound of Formula (XIB):

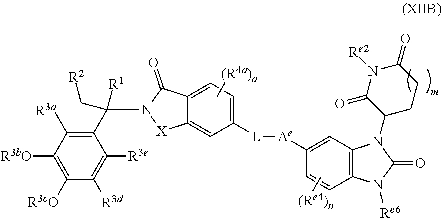

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, R^{e6}, A^{e}, L, X, a, m, and n are each as defined herein.

In still another embodiment, provided herein is a compound of Formula (XIIB):

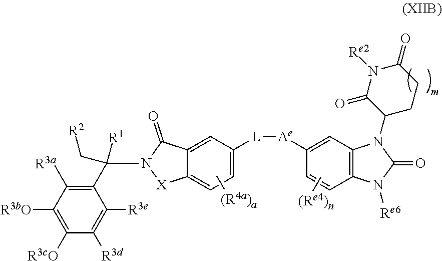

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, R^{e6}, A^{e}, L, X, a, m, and n are each as defined herein.

In one embodiment, provided herein is a compound of Formula (XIIIB):

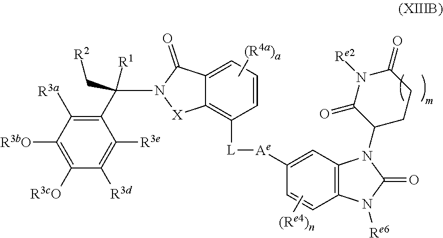

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e2}, R^{e4}, R^{e6}, A^{e}, L, X, a, m, and n are each as defined herein.

In another embodiment, provided herein is a compound of Formula (XIVB):

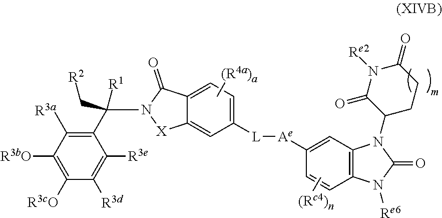

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e2}, R^{e4}, R^{e6}, A^{e}, L, X, a, m, and n are each as defined herein.

In et another embodiment provided herein is a compound of Formula (XVB):

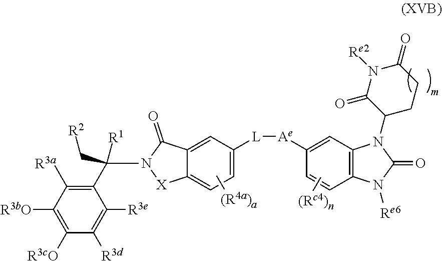

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, R^{e6}, A^{e}, L, X, a, m, and n are each as defined herein.

In certain embodiments, X^{e} is C(R^{e1}), wherein R^{e1} is as defined herein. In certain embodiments, X^{e} is C(H). In certain embodiments, X^{e} is N.

In one embodiment, provided herein is a compound of Formula (IIC):

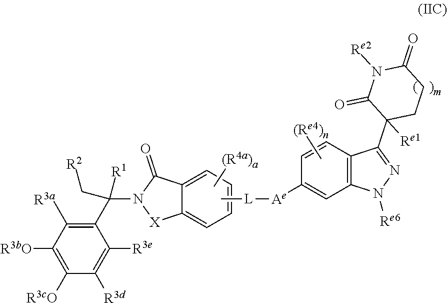

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e1}, R^{e2}, R^{e4}, R^{e6}, A^{e}, L, X, a, m, and n are each as defined herein.

In another embodiment, provided herein is a compound of Formula (IIIC):

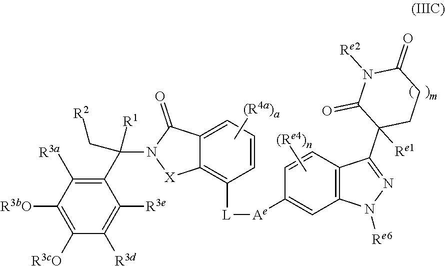

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e1}, R^{e2}, R^{e4}, R^{e6}, A^{e}, L, X, a, m, and n are each as defined herein.

In yet another embodiment, provided herein is a compound of Formula (IVC):

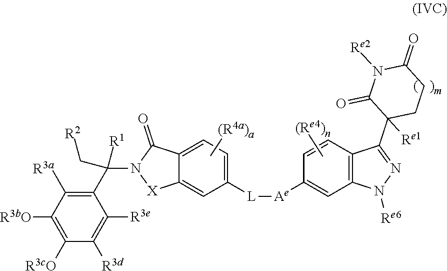

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e1}, R^{e2}, R^{e4}, R^{e6}, A^{e}, L, X, a, m, and n are each as defined herein.

In still another embodiment, provided herein is a compound of Formula (VC):

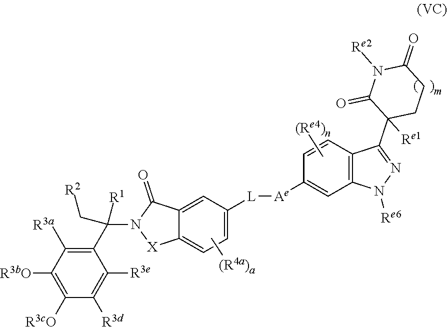

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e1}, R^{e2}, R^{e4}, R^{e6}, A^{e}, L, X, a, m, and n are each as defined herein.

In one embodiment, provided herein is a compound of Formula (VIC):

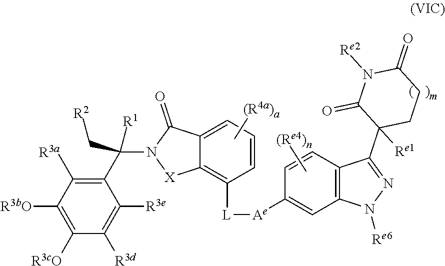

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e1}, R^{e2}, R^{e4}, R^{e6}, A^{e}, L, X, a, m, and n are each as defined herein.

In another embodiment, provided herein is a compound of Formula (VIIC):

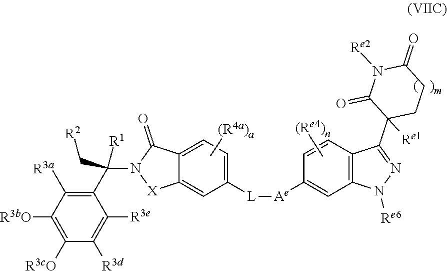

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e1}, R^{e2}, R^{e4}, R^{e6}, A^{e}, L, X, a, m, and n are each as defined herein.

In yet another embodiment, provided herein is a compound of Formula (VIIIC):

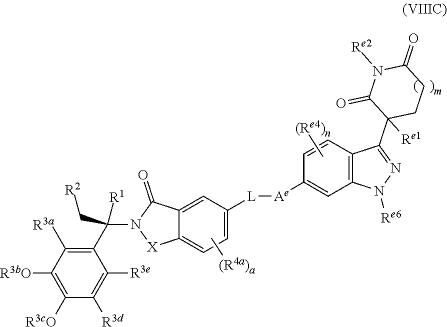

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e1}, R^{e2}, R^{e4}, R^{e6}, A^{e}, L, X, a, m, and n are each as defined herein.

In one embodiment, provided herein is a compound of Formula (IXC):

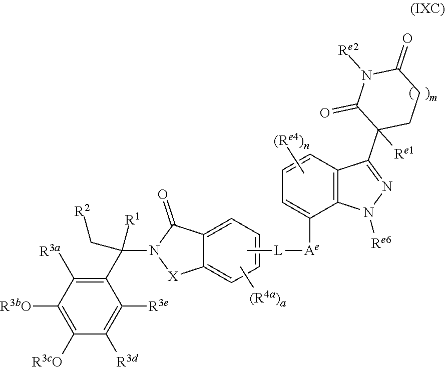

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e1}, R^{e2}, R^{e4}, R^{e6}, A^{e}, L, X, a, m, and n are each as defined herein.

In another embodiment, provided herein is a compound of Formula (XC):

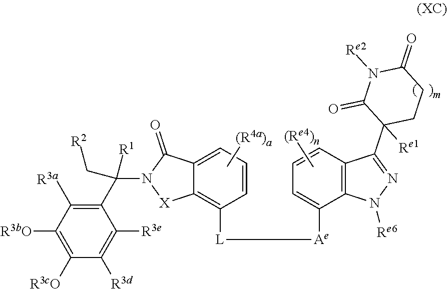

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e1}, R^{e2}, R^{e4}, R^{e6}, A^{e}, L, X, a, m, and n are each as defined herein.

In yet another embodiment, provided herein is a compound of Formula (XIC):

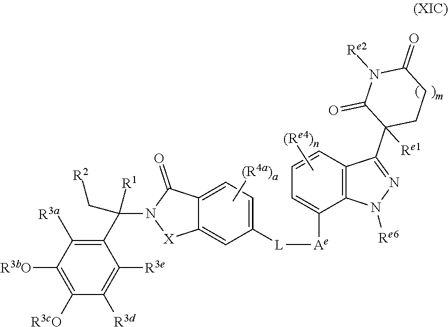

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3e}, R^{3d}, R^{3e}, R^{4a}, R^{e1}, R^{e2}, R^{e4}, R^{e6}, A^{e}, L, X, a, m, and n are each as defined herein.

In still another embodiment, provided herein is a compound of Formula (XIIC):

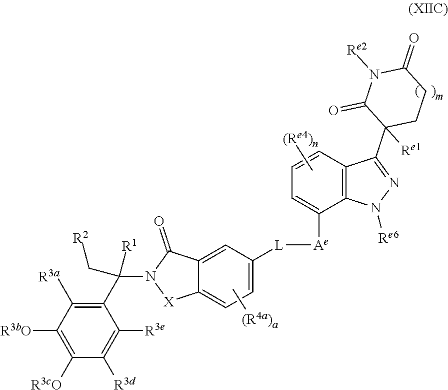

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e1}, R^{e2}, R^{e4}, R^{e6}, A^{e}, L, X, a, m, and n are each as defined herein.

In one embodiment, provided herein is a compound of Formula (XIIIC):

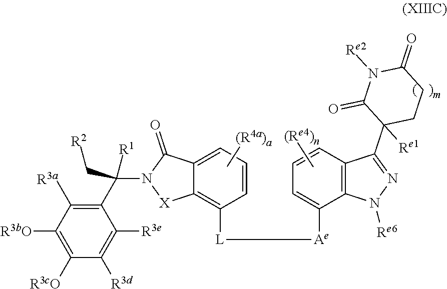

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e1}, R^{e2}, R^{e4}, R^{e6}, A^{e}, L, X, a, m, and n are each as defined herein.

In another embodiment, provided herein is a compound of Formula (XIVC):

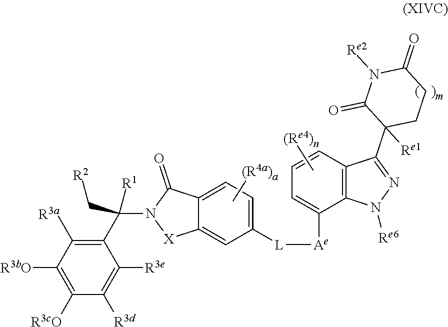

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e1}, R^{e2}, R^{e4}, R^{e6}, A^{e}, L, X, a, m, and n are each as defined herein.

In yet another embodiment, provided herein is a compound of Formula (XVC):

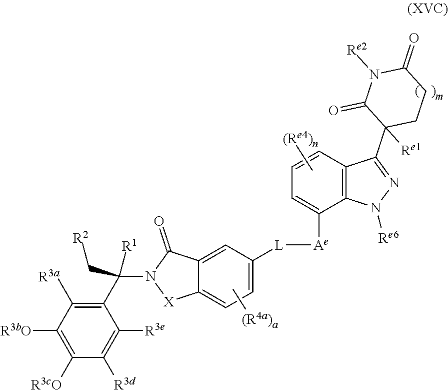

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e1}, R^{e2}, R^{e4}, R^{e6}, A^{e}, L, X, a, m, and n are each as defined herein.

In one embodiment, provided herein is a compound of Formula (XVIC):

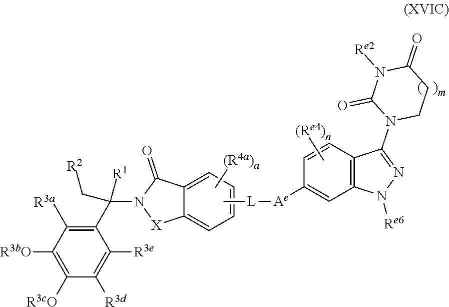

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, R^{e6}, A^{e}, L, X, a, m, and n are each as defined herein.

In another embodiment, provided herein is a compound of Formula (XVIIC):

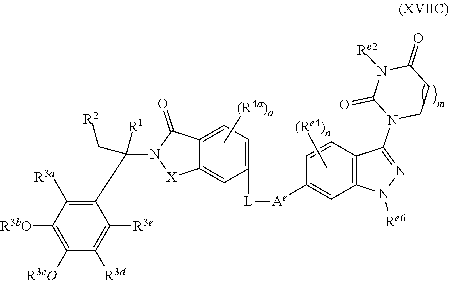

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, R^{e6}, A^{e}, L, X, a, m, and n are each as defined herein.

In yet another embodiment, provided herein is a compound of Formula (XVIIIC):

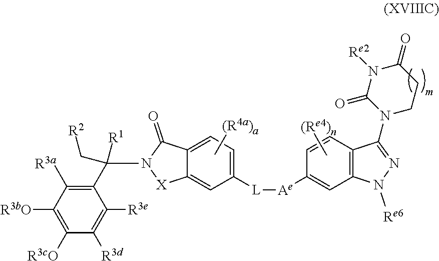

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e2}, R^{e4}, R^{e6}, A^{e}, L, X, a, m, and n are each as defined herein.

In still another embodiment, provided herein is a compound of Formula (XIXC):

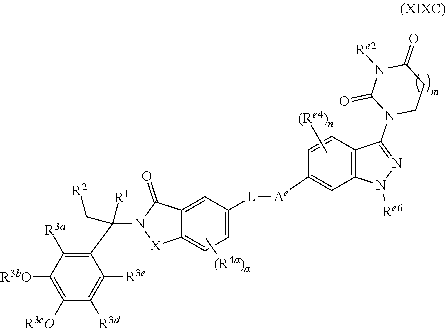

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e2}, R^{e4}, R^{e6}, A^{e}, L, X, a, m, and n are each as defined herein.

In one embodiment, provided herein is a compound of Formula (XXC):

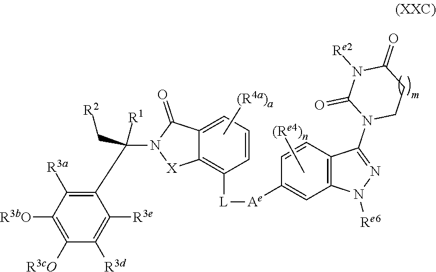

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e2}, R^{e4}, R^{e6}, A^{e}, L, X, a, m, and n are each as defined herein.

In another embodiment, provided herein is a compound of Formula (XXIC):

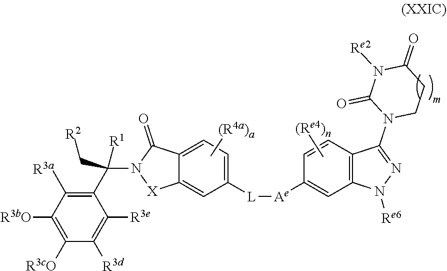

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e2}, R^{e4}, R^{e6}, A^{e}, L, X, a, m, and n are each as defined herein.

In yet another embodiment, provided herein is a compound of Formula (XXIIC):

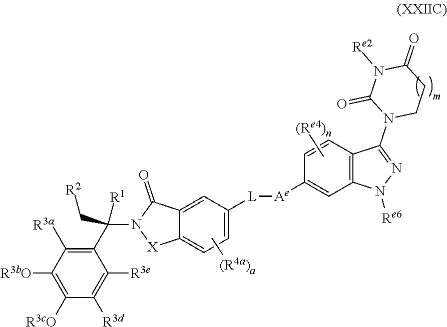

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e2}, R^{e4}, R^{e6}, A^{e}, L, X, a, m, and n are each as defined herein.

In one embodiment, provided herein is a compound of Formula (XXIIIC):

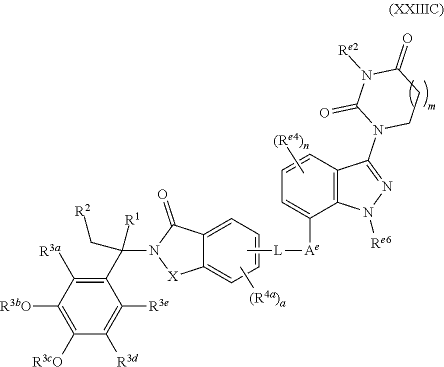

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, R^{e6}, A^{e}, L, X, a, m, and n are each as defined herein.

In another embodiment, provided herein is a compound of Formula (XXIVC):

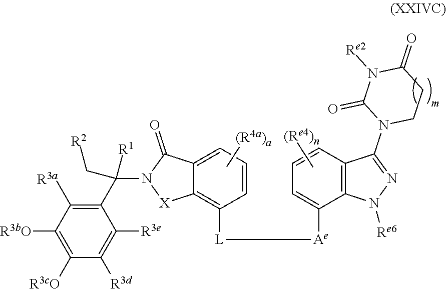

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, R^{e6}, A^{e}, L, X, a, m, and n are each as defined herein.

In yet another embodiment, provided herein is a compound of Formula (XXVC):

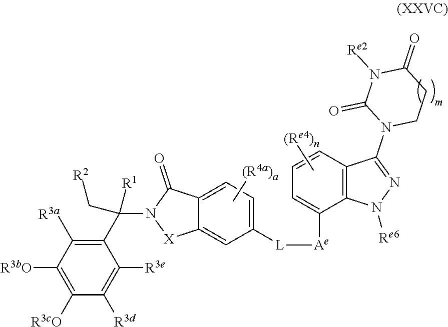

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4e}, R^{e2}, R^{e4}, R^{e6}, A^{e}, L, X, a, m, and n are each as defined herein.

In still another embodiment, provided herein is a compound of Formula (XXVIC):

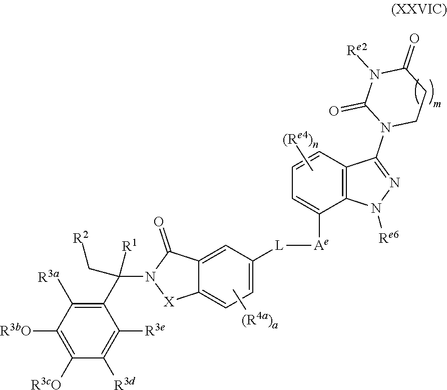

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, R^{e6}, A^{e}, L, X, a, m, and n are each as defined herein.

In one embodiment, provided herein is a compound of Formula (XXVIIC):

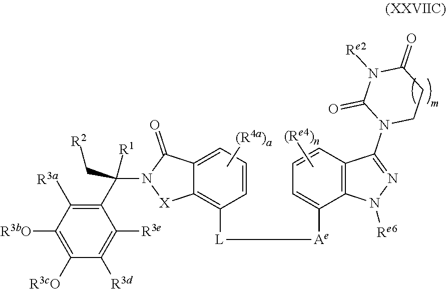

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, R^{e6}, A^{e}, L, X, a, m, and n are each as defined herein.

In another embodiment, provided herein is a compound of Formula (XXVIIIC):

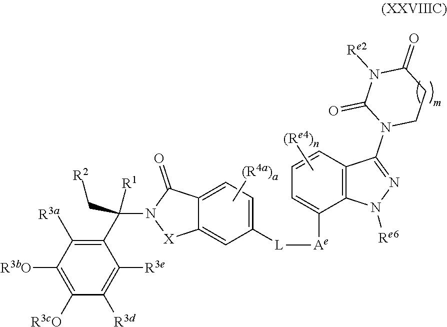

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, R^{e6}, A^{e}, L, X, a, m, and n are each as defined herein.

In yet another embodiment, provided herein is a compound of Formula (XXIXC):

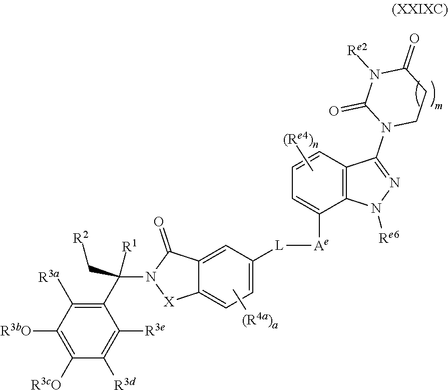

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e2}, R^{e4}, R^{e6}, A^{e}, L, X, a, m, and n are each as defined herein.

In one embodiment, provided herein is a compound of Formula (IID):

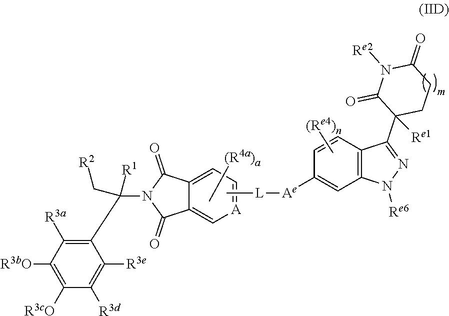

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e1}, R^{e2}, R^{e4}, R^{e6}, A, A^{e}, L, a, m, and n are each as defined herein.

In another embodiment, provided herein is a compound of Formula (IIID):

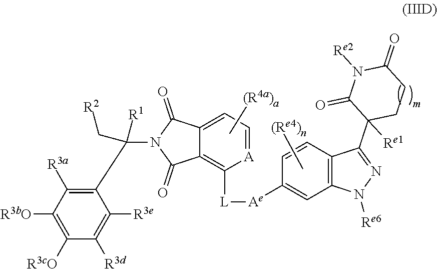

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof, wherein R¹, R², R^{3a}, R^{3b}, R^{3c}, R^{3d}, R^{3e}, R^{4a}, R^{e1}, R^{e2}, R^{e4}, R^{e6}, A, A^{e}, L, a, m, and n are each as defined herein.

In yet another embodiment, provided herein is a compound of Formula (IVD):

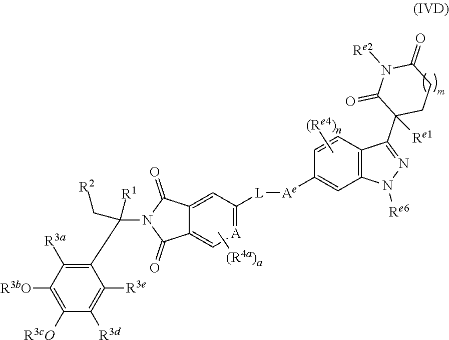

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e1}, R^{e2}, R^{e4}, R^{e6}, A, A^{e}, L, a, m, and n are each as defined herein.

In yet another embodiment, provided herein is a compound of Formula (VD):

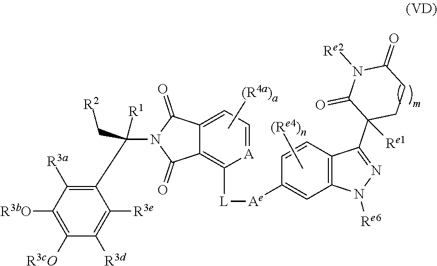

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof, wherein R¹, R², R^{3a}, R^{3b}, R^{3c}, R^{3d}, R^{3c}, R^{4a}, R^{c1}, R^{c2}, R^{c4}, R^{c6}, A, A^{c}, L, a, m, and n are each as defined herein.

In still another embodiment, provided herein is a compound of Formula (VID):

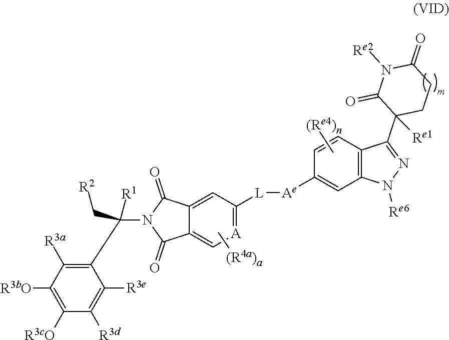

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3c}, R^{4a}, R^{c1}, R^{c2}, R^{c4}, R^{c6}, A, A^{e}, L, a, m, and n are each as defined herein.

In one embodiment, provided herein is a compound of Formula (VIID):

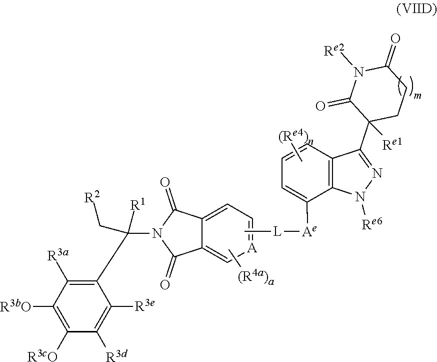

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof, wherein R¹, R², R^{3a}, R^{3b}, R^{3c}, R^{3d}, R^{3e}, R^{4a}, R^{e1}, R^{e2}, R^{e4}, R^{e6}, A, A^{e}, L, a, m, and n are each as defined herein.

In another embodiment, provided herein is a compound of Formula (VIID):

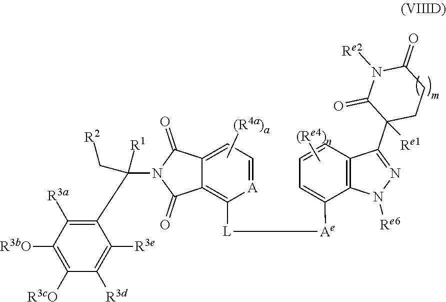

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e1}, R^{e2}, R^{e4}, R^{e6}, A, A^{e}, L, a, m, and n are each as defined herein.

In yet another embodiment, provided herein is a compound of Formula (IXD):

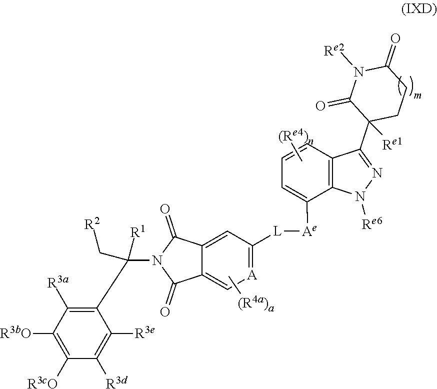

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof, wherein R¹, R², R^{3a}, R^{3b}, R^{3c}, R^{3d}, R^{3e}, R^{4a}, R^{c1}, R^{c2}, R^{c4}, R^{c6}, A, A^{c}, L, a, m, and n are each as defined herein.

In yet another embodiment, provided herein is a compound of Formula (XD):

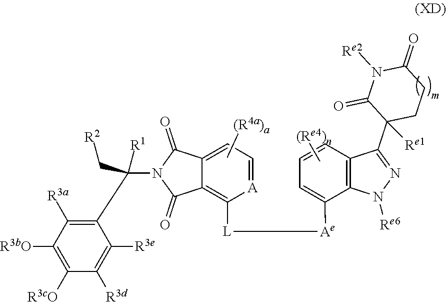

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e1}, R^{e2}, R^{e4}, R^{e6}, A, A^{e}, L, a, m, and n are each as defined herein.

In still another embodiment, provided herein is a compound of Formula (XID):

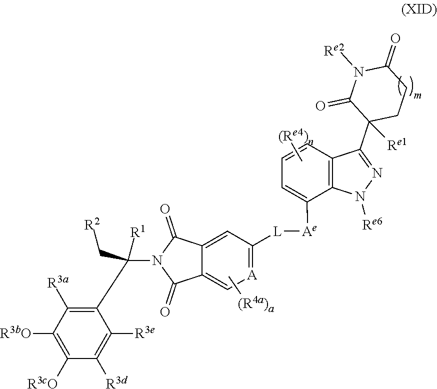

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof, wherein R¹, R², R^{3a}, R^{3b}, R^{3c}, R^{3d}, R^{3c}, R^{4a}, R^{c1}, R^{c2}, R^{c4}, R^{c6}, A, A^{c}, L, a, m, and n are each as defined herein.

In one embodiment, provided herein is a compound of Formula (XIID):

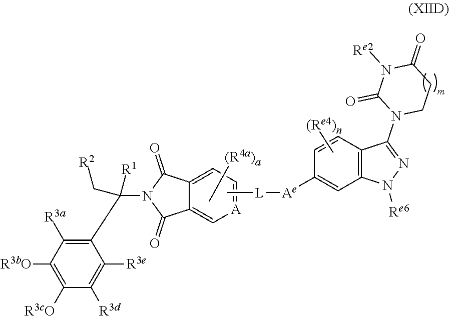

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, R^{e6}, A, A^{e}, L, a, m, and n are each as defined herein.

In another embodiment, provided herein is a compound of Formula (XIIID):

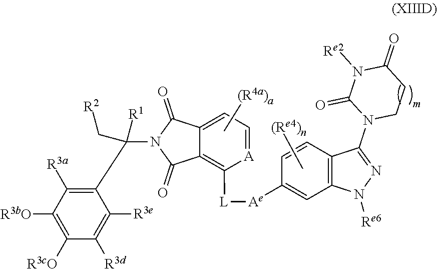

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof, wherein R¹, R², R^{3a}, R^{3b}, R^{3c}, R^{3d}, R^{3e}, R^{4a}, R^{e2}, R^{e4}, R^{e6}, A, A^{e}, L, a, m, and n are each as defined herein.

In yet another embodiment, provided herein is a compound of Formula (XIVD):

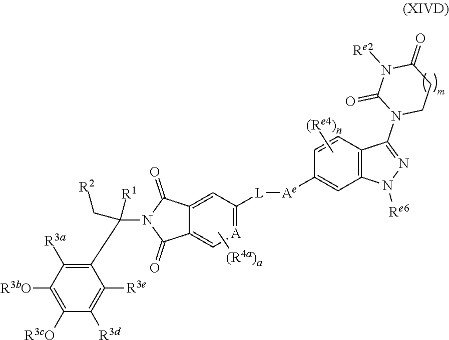

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, R^{e6}, A, A^{e}, L, a, m, and n are each as defined herein.

In yet another embodiment, provided herein is a compound of Formula (XVD):

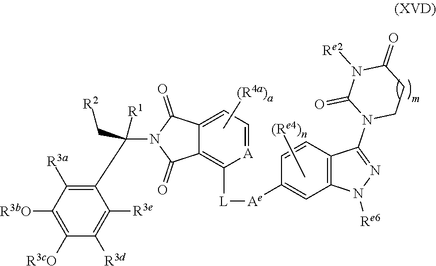

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof, wherein R¹, R², R^{3a}, R^{3b}, R^{3c}, R^{3d}, R^{3e}, R^{4a}, R^{e2}, R^{e4}, R^{e6}, A, A^{e}, L, a, m, and n are each as defined herein.

In still another embodiment, provided herein is a compound of Formula (XVID):

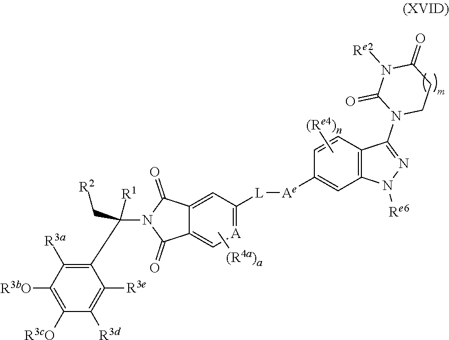

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, R^{e6}, A, A^{e}, L, a, m, and n are each as defined herein.

In one embodiment, provided herein is a compound of Formula (XVIID):

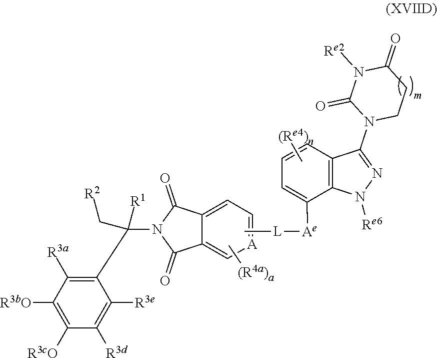

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof, wherein R¹, R², R^{3a}, R^{3b}, R^{3c}, R^{3d}, R^{3e}, R^{4a}, R^{e2}, R^{e4}, R^{e6}, A, A^{e}, L, a, m, and n are each as defined herein.

In another embodiment, provided herein is a compound of Formula (XVIIID):

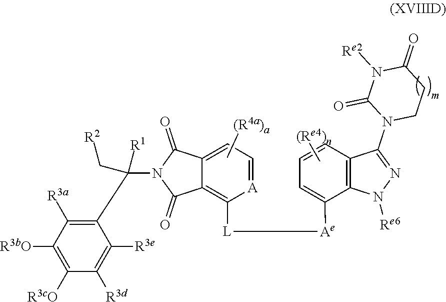

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof, wherein R¹, R², R^{3a}, R^{3b}, R^{3c}, R^{3d}, R^{3e}, R^{4a}, R^{e2}, R^{e4}, R^{e6}, A, A^{e}, L, a, m, and n are each as defined herein.

In yet another embodiment, provided herein is a compound of Formula (XIXD):

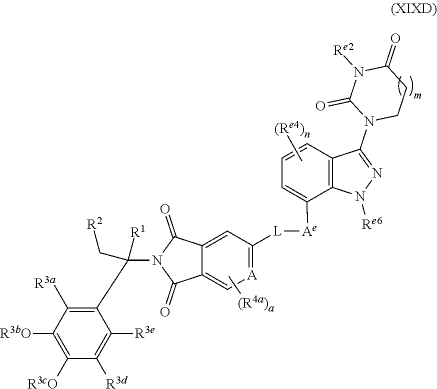

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof, wherein R¹, R², R^{3a}, R^{3b}, R^{3c}, R^{3d}, R^{3e}, R^{4a}, R^{c2}, R^{c4}, R^{c6}, A, A^{c}, L, a, m, and n are each as defined herein.

In yet another embodiment, provided herein is a compound of Formula (XXD):

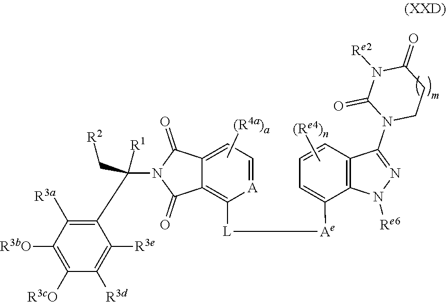

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R^{3a}, R^{3b}, R^{3c}, R^{3d}, R^{3e}, R^{4a}, R^{e2}, R^{e4}, R^{e6}, A, A^{e}, L, a, m, and n are each as defined herein.

In still another embodiment, provided herein is a compound of Formula (XXID):

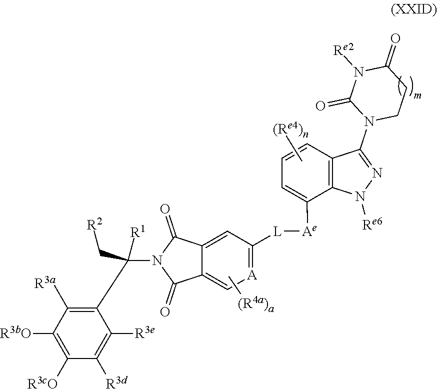

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof, wherein R¹, R², R^{3a}, R^{3b}, R^{3c}, R^{3d}, R^{3e}, R^{4a}, R^{c2}, R^{c4}, R^{c6}, A, A^{c}, L, a, m, and n are each as defined herein.

In certain embodiments, R¹ is hydrogen. In certain embodiments, R¹ is deuterium.

In certain embodiments, R² is cyano. In certain embodiments, R² is —S(O₂)R^{2a}, wherein R^{2a} is as defined herein. In certain embodiments, R² is C₁₋₆ alkylsulfonyl or C₃₋₁₀ cycloalkylsulfonyl, each optionally substituted with one or more substituents Q. In certain embodiments, R² is C₁₋₆ alkylsulfonyl, optionally substituted with one or more substituents Q. In certain embodiments, R² is methylsulfonyl or cyclopropylmethylsulfonyl, each optionally substituted with one or more substituents Q. In certain embodiments, R² is methylsulfonyl. In certain embodiments, R² is C₃₋₁₀ cycloalkylsulfonyl, optionally substituted with one or more substituents Q. In certain embodiments, R² is monocyclic C₃₋₁₀ cycloalkyl-sulfonyl, optionally substituted with one or more substituents Q. In certain embodiments, R² is cyclopropyl-sulfonyl, optionally substituted with one or more substituents Q. In certain embodiments, R² is cyclopropyl-sulfonyl.

In certain embodiments, R⁴ is hydrogen. In certain embodiments, R⁴ is R^{4a}, which is as defined herein.

In certain embodiments, R^{2a} is (i) C₁₋₆ alkyl, C₃₋₁₀ cycloalkyl, heteroaryl, or heterocyclyl, each of which is optionally substituted with one or more substituents Q; or (ii) —OR^{1a} or —NR^{1b}R^{1c}; wherein R^{1a}, R^{1b}, and R^{1c} are each as defined herein. In certain embodiments, R^{2a} is C₁₋₆ alkyl or C₃₋₁₀ cycloalkyl, each of which is optionally substituted with one or more substituents Q. In certain embodiments, R^{2a} is C₁₋₆ alkyl, optionally substituted with one or more substituents Q. In certain embodiments, R^{2a} is methyl or cyclopropylmethyl. In certain embodiments, R^{2a} is methyl. In certain embodiments, R^{2a} is C₃₋₁₀ cycloalkyl, optionally substituted with one or more substituents Q. In certain embodiments, R^{2a} is monocyclic C₃₋₁₀ cycloalkyl, optionally substituted with one or more substituents Q. In certain embodiments, R^{2a} is bicyclic C₄₋₁₀ cycloalkyl, optionally substituted with one or more substituents Q. In certain embodiments, R^{2a} is bridged, fused, or spiro C₄₋₁₀ cycloalkyl, each optionally substituted with one or more substituents Q. In certain embodiments, R^{2a} is cyclopropyl. In certain embodiments, R^{2a} is methyl, cyclopropylmethyl, or cyclopropyl.

In certain embodiments, R^{3a} is hydrogen or deuterium. In certain embodiments, R^{3a} is hydrogen. In certain embodiments, R^{3a} is deuterium.

In certain embodiments, R^{3b} is C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each of which is optionally substituted with one or more substituents Q. In certain embodiments, R^{3b} is C₁₋₆ alkyl, C₁₋₆ heteroalkyl, or C₃₋₁₀ cycloalkyl, each of which is optionally substituted with one or more substituents Q. In certain embodiments, R^{3b} is C₁₋₆ alkyl or C₃₋₁₀ cycloalkyl, optionally substituted with one or more substituents Q. In certain embodiments, R^{3b} is C₁₋₆ alkyl, optionally substituted with one or more substituents Q. In certain embodiments, R^{3b} is methyl, ethyl, or cyclopropylmethyl. In certain embodiments, R^{3b} is methyl. In certain embodiments, R^{3b} is ethyl. In certain embodiments, R^{3b} is C₃₋₁₀ cycloalkyl, optionally substituted with one or more substituents Q. In certain embodiments, R^{3b} is monocyclic C₃₋₁₀ cycloalkyl, optionally substituted with one or more substituents Q. In certain embodiments, R^{3b} is bicyclic C₄₋₁₀ cycloalkyl, optionally substituted with one or more substituents Q. In certain embodiments, R^{3b} is bridged, fused, or spiro C₄₋₁₀ cycloalkyl, each optionally substituted with one or more substituents Q. In certain embodiments, R^{3b} is cyclopropyl. In certain embodiments, R^{3b} is methyl, ethyl, cyclopropylmethyl, or cyclopropyl.

In certain embodiments, R^{3c} is C₁₋₆ alkyl, C₁₋₆ heteroalkyl, C₃₋₁₀ cycloalkyl, C₆₋₁₄ aryl, C₇₋₁₅ aralkyl, heteroaryl, or heterocyclyl, each of which is optionally substituted with one or more substituents Q. In certain embodiments, R is C₁₋₆ alkyl, C₁₋₆ heteroalkyl, or C₃₋₁₀ cycloalkyl, each of which is optionally substituted with one or more substituents Q. In certain embodiments, R^{3c} is C₁₋₆ alkyl or C₃₋₁₀ cycloalkyl, each optionally substituted with one or more substituents Q. In certain embodiments, R^{3c} is C₁₋₆ alkyl, optionally substituted with one or more substituents Q. In certain embodiments, R^{3c} is methyl, ethyl, or cyclopropylmethyl. In certain embodiments, R^{3c} is methyl. In certain embodiments, R^{3c} is ethyl. In certain embodiments, R^{3c} is C₃₋₁₀ cycloalkyl, optionally substituted with one or more substituents Q. In certain embodiments, R^{3c} is monocyclic C₃₋₁₀ cycloalkyl, optionally substituted with one or more substituents Q. In certain embodiments, R^{3c} is bicyclic C₄₋₁₀ cycloalkyl, optionally substituted with one or more substituents Q. In certain embodiments, R^{3c} is bridged, fused, or spiro C₄₋₁₀ cycloalkyl, each optionally substituted with one or more substituents Q. In certain embodiments, R^{3c} is cyclopropyl. In certain embodiments, R^{3c} is methyl, ethyl, cyclopropylmethyl, or cyclopropyl.

In certain embodiments, R^{3b} and R^{3c} are each independently C₁₋₆ alkyl, optionally substituted with one or more substituents Q. In certain embodiments, R^{3b} and R^{3c} are each independently methyl or ethyl. In certain embodiments, R^{3b} is methyl and R^{3c} is ethyl. In certain embodiments, R^{3A} is ethyl and R^{3c} is methyl.

In certain embodiments, R^{3d} is hydrogen or deuterium. In certain embodiments, R^{3d} is hydrogen. In certain embodiments, R^{3d} is deuterium.

In certain embodiments, R^{3e} is hydrogen or deuterium. In certain embodiments, R^{3e} is hydrogen. In certain embodiments, R^{3d} is deuterium.

In certain embodiments, R^{3a}, R^{3d}, and R^{3e} are each hydrogen.

In certain embodiments, R^{4a} is deuterium, halo, —NR^{1b}R^{1c}, or —NR^{1a}C(O)R^{1d}, wherein R^{1a}, R^{1b}, R^{1c}, and R^{1d} are each as defined herein. In certain embodiments, R^{4a} is deuterium. In certain embodiments, R^{4a} is halo. In certain embodiments, R^{4a} is fluoro or chloro. In certain embodiments, R^{4a} is —NR^{1b}R^{1c}, wherein R^{1b} and R^{1c} are each as defined herein. In certain embodiments, R^{4a} is —NH₂. In certain embodiments, R^{4a} is —NR^{1a}C(O)R^{1d}, wherein R^{1a} and R^{1d} are each as defined herein. In certain embodiments, R^{4a} is —NHC(O)R^{1d}, wherein R^{1d} is C₁₋₆ alkyl optionally substituted with one or more substituents Q. In certain embodiments, R^{4a} is —NHC(O)CH₃.

In certain embodiments, A is C(R⁴), wherein R⁴ is as defined herein. In certain embodiments, A is C(H). In certain embodiments, A is N.

In certain embodiments, X is —CH₂—. In certain embodiments, X is —C(O)—.

In certain embodiments, a is an integer of 0, 1, or 2. In certain embodiments, a is an integer of 0. In certain embodiments, a is an integer of 1. In certain embodiments, a is an integer of 2.

In one embodiment,
R¹ is hydrogen or deuterium;
R² is cyano;
R^{3a}, R^{3d}, and R^{3e} are each hydrogen;
R^{3b} and R^{3c} are each independently C₁₋₆ alkyl;
R^{4a}, if present, is hydrogen or —NHC(O)R^{1d}, wherein R^{1d} is C₁₋₆ alkyl;
A is C(H) or N; and
a is an integer of 0 or 1;
wherein each alkyl and cycloalkyl is optionally substituted with one or more substituents Q.

In another embodiment,
R¹ is hydrogen or deuterium;
R² is cyano;
R^{3a}, R^{3d}, and R^{3e} are each hydrogen;
R^{3b} and R^{3c} are each independently methyl or ethyl;
R^{4a}, if present, is hydrogen or —NHC(O)CH₃;
A is C(H) or N; and
a is an integer of 0 or 1.

In yet another embodiment,
R¹ is hydrogen or deuterium;
R² is —S(O₂)R^{2a};
R^{2a} is C₁₋₆ alkyl or C₃₋₁₀ cycloalkyl;
R^{3a}, R^{3b}, and R^{3e} are each hydrogen;
R^{3b} and R^{3c} are each independently C₁₋₆ alkyl;
R^{4a}, if present, is hydrogen or —NHC(O)R^{1d}, wherein R^{1d} is C₁₋₆ alkyl;
A is C(H) or N; and
a is an integer of 0 or 1;
wherein each alkyl and cycloalkyl is optionally substituted with one or more substituents Q.

In still another embodiment,
R¹ is hydrogen or deuterium;
R² is —S(O₂)R^{2a};
R^{2a} is methyl or cyclopropylmethyl;
R^{3a}, R^{3d}, and R^{3e} are each hydrogen;
R^{3b} and R^{3c} are each independently methyl or ethyl;
R^{4a}, if present, is hydrogen or —NHC(O)CH₃;
A is C(H) or N; and
a is an integer of 0 or 1.

In one embodiment,
R¹ is hydrogen or deuterium;
R² is cyano;
R^{3a}, R^{3d}, and R^{3e} are each hydrogen;
R^{3b} and R^{3c} are each independently C₁₋₆ alkyl;
R^{4a}, if present, is hydrogen or —NHC(O)R^{1d}, wherein R^{1d} is C₁₋₆ alkyl;
X is —CH₂— or —C(O)—; and
a is an integer of 0 or 1;
wherein each alkyl and cycloalkyl is optionally substituted with one or more substituents Q.

In another embodiment,
R¹ is hydrogen or deuterium;
R² is cyano;
R^{3a}, R^{3d}, and R^{3e} are each hydrogen;
R^{3b} and R^{3c} are each independently methyl or ethyl;
R^{4a}, if present, is hydrogen or —NHC(O)CH₃;
X is —CH₂— or —C(O)—; and
a is an integer of 0 or 1.

In yet another embodiment,
R¹ is hydrogen or deuterium;
R² is —S(O₂)R^{2a};
R^{2a} is C₁₋₆ alkyl or C₃₋₁₀ cycloalkyl;
R^{3a}, R^{3d}, and R^{3c} are each hydrogen;
R^{3b} and R^{3c} are each independently C₁₋₆ alkyl;
R^{4a}, if present, is hydrogen or —NHC(O)R^{1d}, wherein R^{1d} is C₁₋₆ alkyl;
X is —CH₂— or —C(O)—; and
a is an integer of 0 or 1;
wherein each alkyl and cycloalkyl is optionally substituted with one or more substituents Q.

In still another embodiment,
R¹ is hydrogen or deuterium;
R² is —S(O₂)R^{2a};
R^{2a} is methyl or cyclopropylmethyl;
R^{3a}, R^{3d}, and R^{3e} are each hydrogen;
R^{3b} and R^{3e} are each independently methyl or ethyl;
R^{4a}, if present, is hydrogen or —NHC(O)CH₃;
X is —CH₂— or —C(O)—; and
a is an integer of 0 or 1.

In certain embodiments, A^{e} is (i) C₁₋₆ alkylene, C₂₋₆ alkynylene, C₃₋₁₀ cycloalkylene, or heterocyclylene, each optionally substituted with one or more substituents Q; or (ii) —O— or —NHCH₂CH₂—. In certain embodiments, A^{e} is C₁₋₆ alkylene, C₂₋₆ alkynylene, C₃₋₁₀ cycloalkylene, or heterocyclylene, each optionally substituted with one or more substituents Q. In certain embodiments, A^{e} is C₂₋₆ alkynylene, C₃₋₁₀ cycloalkylene, or heterocyclylene, each optionally substituted with one or more substituents Q. In certain embodiments, A^{e} is C₃₋₁₀ cycloalkylene or heterocyclylene, each optionally substituted with one or more substituents Q.

In certain embodiments, A^{e} is C₁₋₆ alkylene, optionally substituted with one or more substituents Q. In certain embodiments, A^{e} is methanediyl, ethanediyl, or propanediyl. In certain embodiments, A^{e} is methanediyl, ethane-1,2-diyl, or propane-1,3-diyl. In certain embodiments, A^{e} is C₂₋₆ alkynylene, optionally substituted with one or more substituents Q. In certain embodiments, A^{e} is ethynediyl or butyne-1,4-diyl. In certain embodiments, A^{e} is ethyne-1,2-diyl or 1-butyne-1,4-diyl.

In certain embodiments, A^{e} is C₃₋₁₀ cycloalkylene, optionally substituted with one or more substituents Q. In certain embodiments, A^{e} is monocyclic C₃₋₁₀ cycloalkylene, optionally substituted with one or more substituents Q. In certain embodiments, A^{e} is bicyclic C₄₋₁₀ cycloalkylene, optionally substituted with one or more substituents Q. In certain embodiments, A^{e} is bridged, fused, or spiro C₄₋₁₀ cycloalkylene, each optionally substituted with one or more substituents Q. In certain embodiments, A^{e} is bridged C₄₋₁₀ cycloalkylene, optionally substituted with one or more substituents Q. In certain embodiments, A^{e} is fused C₄₋₁₀ cycloalkylene, optionally substituted with one or more substituents Q. In certain embodiments, A^{e} is spiro C₅₋₁₀ cycloalkylene, optionally substituted with one or more substituents Q. In certain embodiments, A^{e} is cyclohexanediyl. In certain embodiments, A^{e} is cyclohexane-1,4-diyl.

In certain embodiments, A^{e} is heterocyclylene, optionally substituted with one or more substituents Q. In certain embodiments, A^{e} is monocyclic heterocyclylene, optionally substituted with one or more substituents Q. In certain embodiments, A^{e} is 3-, 4-, 5-, 6-, or 7-membered heterocyclylene, each optionally substituted with one or more substituents Q. In certain embodiments, A^{e} is 3-membered heterocyclylene, optionally substituted with one or more substituents Q. In certain embodiments, A^{e} is 4-membered heterocyclylene, optionally substituted with one or more substituents Q. In certain embodiments, A^{e} is 5-membered heterocyclylene, optionally substituted with one or more substituents Q. In certain embodiments, A^{e} is 6-membered heterocyclylene, optionally substituted with one or more substituents Q. In certain embodiments, A^{e} is 7-membered heterocyclylene, optionally substituted with one or more substituents Q. In certain embodiments, A^{e} is azetidindiyl, piperidindiyl, or piperazindiyl. In certain embodiments, AC is azetidin-1,3-diyl, piperidin-1,4-diyl, or piperazin-1,4-diyl. In certain embodiments, A^{e} is bicyclic heterocyclylene, optionally substituted with one or more substituents Q. In certain embodiments, A^{e} is bridged, fused, or spiro heterocyclylene, each optionally substituted with one or more substituents Q. In certain embodiments, A^{e} is bridged heterocyclylene, optionally substituted with one or more substituents Q. In certain embodiments, A^{e} is fused heterocyclylene, optionally substituted with one or more substituents Q. In certain embodiments, A^{e} is spiro heterocyclylene, optionally substituted with one or more substituents Q. In certain embodiments, A^{e} is azaspiro[3.3]heptandiyl or azaspiro[5.5]undecandiyl. In certain embodiments, A^{e} is 2-azaspiro[3.3]heptan-2,6-diyl or 3-azaspiro[5.5]undecan-3,9-diyl. In certain embodiments, A^{e} is —O—. In certain embodiments, A^{e} is —NHCH₂CH₂—.

In certain embodiments, A^{e} is methanediyl, ethane-1,2-diyl, propane-1,3-diyl, ethyne-1,2-diyl, 1-butyne-1,4-diyl, cyclohexane-1,4-diyl, azetidin-1,3-diyl, piperidin-1,4-diyl, piperazin-1,4-diyl, 2-azaspiro[3.3]-heptan-2,6-diyl, 3-azaspiro[5.5]undecan-3,9-diyl, —O—, or

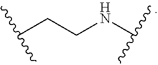 [SMILES: *CCN*]

In certain embodiments, A^{e} is cyclohexane-1,4-diyl, azetidin-1,3-diyl, piperidin-1,4-diyl, piperazin-1,4-diyl, 2-azaspiro[3.3]-heptan-2,6-diyl, or 3-azaspiro[5.5]undecan-3,9-diyl. In certain embodiments, A^{e} is azetidin-1,3-diyl, piperidin-1,4-diyl, piperazin-1,4-diyl, 2-azaspiro[3.3]-heptan-2,6-diyl, or 3-azaspiro[5.5]-undecan-3,9-diyl.

In certain embodiments, R^{e1} is (i) hydrogen, deuterium, or halo; or (ii) C₁₋₆ alkyl, optionally substituted with one or more substituents Q. In certain embodiments, R^{e1} is (i) hydrogen or deuterium; or (ii) C₁₋₆ alkyl, optionally substituted with one or more substituents Q. In certain embodiments, R^{e1} is hydrogen. In certain embodiments, R^{e1} is deuterium. In certain embodiments, R^{e1} is C₁₋₆ alkyl, optionally substituted with one or more substituents Q. In certain embodiments, R^{e1} is hydrogen, deuterium, fluoro, chloro, methyl, or ethyl. In certain embodiments, R^{e1} is methyl.

In certain embodiments, R^{e2} is hydrogen. In certain embodiments, R^{e2} is C₁₋₆ alkyl, optionally substituted with one or more substituents Q.

In certain embodiments, R^{e3} is hydrogen. In certain embodiments, R^{e3} is C₁₋₆ alkyl, optionally substituted with one or more substituents Q. In certain embodiments, R^{e3} is methyl.

In certain embodiments, R^{e4} is halo or —OR^{1a}, wherein R^{1a} is as defined herein. In certain embodiments, R^{e4} is halo. In certain embodiments, R^{e4} is fluoro or chloro. In certain embodiments, R^{e4} is fluoro. In certain embodiments, R^{e4} is —OR^{1a}, wherein R^{1a} is as defined herein. In certain embodiments, R^{e4} is C₁₋₆ alkoxy, optionally substituted with one or more substituents Q. In certain embodiments, R^{e4} is methoxy. In certain embodiments, R^{e4} is fluoro or methoxy.

In certain embodiments, R^{e5} is hydrogen. In certain embodiments, R^{e5} is oxo.

In certain embodiments, R^{e6} is hydrogen. In certain embodiments, R^{e6} is C₁₋₆ alkyl, optionally substituted with one or more substituents Q. In certain embodiments, R^{e6} is methyl.

In certain embodiments, m is an integer of 0. In certain embodiments, m is an integer of 1. In certain embodiments, m is an integer of 2.

In certain embodiments, n is an integer of 0. In certain embodiments, n is an integer of 1. In certain embodiments, n is an integer of 2. In certain embodiments, n is an integer of 3. In certain embodiments, n is an integer of 4. In certain embodiments, n is an integer of 0, 1, or 2.

In certain embodiments, L is a linker having the structure of —Zⁿ—(Rⁿ—Zⁿ)_{z}—, wherein:
each Rⁿ is independently C₁₋₆ alkylene, C₂₋₆ alkenylene, C₂₋₆ alkynylene, C₃₋₁₀ cycloalkylene, C₆₋₁₄ arylene, heteroarylene, or heterocyclylene, each of which is optionally substituted with one or more substituents Q;
each Zⁿ is independently a bond, —C(O)—, —C(O)O—, —C(O)NR^{1b}—, —C(O)S—, —C(NR^{1a})NR^{1b}—, —C(S)—, —C(S)O—, —C(S)NR^{1b}—, —O—, —OC(O)O—, —OC(O)NR^{1b}—, —OC(O)S—, —OC(NR^{1a})NR^{1b}—, —OC(S)O—, —OC(S)NR^{1b}—, —OS(O)—, —OS(O)₂—, —OS(O)NR^{1b}—, —OS(O)₂NR^{1b}—, —NR^{1b}—, —NR^{1a}C(O)NR^{1b}—, —NR^{1a}C(O)S—, —NR^{1a}C(NR^{1d})NR^{1b}—, —NR^{1a}C(S)NR^{1b}—, —NR^{1a}S(O)NR^{1b}—, —NR^{1a}S(O)₂NR^{1b}—, —S—, —S(O)—, —S(O)₂—, —S(O)NR^{1b}—, or —S(O)₂NR^{1b}—; wherein each R^{1a}, R^{1b}, and R^{1d} is as defined herein; and
z is an integer of 1, 2, 3, 4, or 5.

In certain embodiments, each Rⁿ is independently C₁₋₆ alkylene, C₂₋₆ alkynylene, C₃₋₆ cycloalkylene, C₆₋₁₄ arylene, heteroarylene, or heterocyclylene, each optionally substituted with one or more substituents Q. In certain embodiments, each Rⁿ is independently C₁₋₆ alkylene, C₂₋₆ alkynylene, C₃₋₆ cycloalkylene, or heterocyclylene, each optionally substituted with one or more substituents Q. In certain embodiments, each Rⁿ is independently C₁₋₆ alkylene or heterocyclylene, each optionally substituted with one or more substituents Q. In certain embodiments, each Rⁿ is independently C₁₋₆ alkylene, optionally substituted with one or more substituents Q. In certain embodiments, each Rⁿ is independently methanediyl, ethanediyl, propanediyl, butanediyl, pentanediyl, or hexanediyl, each optionally substituted with one or more substituents Q. In certain embodiments, each Rⁿ is independently methanediyl, ethane-1,2-diyl, propane-1,3-diyl, butane-1,4-diyl, pentane-1,5-diyl, or hexane-1,6-diyl.

In certain embodiments, each Rⁿ is independently heterocyclylene, optionally substituted with one or more substituents Q. In certain embodiments, each Rⁿ is independently monocyclic heterocyclylene, optionally substituted with one or more substituents Q. In certain embodiments, each Rⁿ is independently 3-, 4-, 5-, 6-, or 7-membered heterocyclylene, each optionally substituted with one or more substituents Q. In certain embodiments, each Rⁿ is independently 3-membered heterocyclylene, optionally substituted with one or more substituents Q. In certain embodiments, each Rⁿ is independently 4-membered heterocyclylene, optionally substituted with one or more substituents Q. In certain embodiments, each Rⁿ is independently 5-membered heterocyclylene, optionally substituted with one or more substituents Q. In certain embodiments, each Rⁿ is independently 6-membered heterocyclylene, optionally substituted with one or more substituents Q. In certain embodiments, each Rⁿ is independently 7-membered heterocyclylene, optionally substituted with one or more substituents Q. In certain embodiments, each Rⁿ is independently azetidindiyl, piperidindiyl, or piperazindiyl. In certain embodiments, each Rⁿ is independently azetidin-1,3-diyl, piperidindiyl, or piperazin-1,4-diyl. In certain embodiments, each Rⁿ is independently bicyclic heterocyclylene, optionally substituted with one or more substituents Q. In certain embodiments, each Rⁿ is independently bridged, fused, or spiro heterocyclylene, each optionally substituted with one or more substituents Q. In certain embodiments, each Rⁿ is independently bridged heterocyclylene, optionally substituted with one or more substituents Q. In certain embodiments, each Rⁿ is independently fused heterocyclylene, optionally substituted with one or more substituents Q. In certain embodiments, each Rⁿ is independently spiro heterocyclylene, optionally substituted with one or more substituents Q.

In certain embodiments, each Rⁿ is independently methanediyl, ethanediyl, propanediyl, butanediyl, pentanediyl, hexanediyl, ethynediyl, propynediyl, pentynediyl, cyclobutanediyl, cyclopentanediyl, cyclohexanediyl, cycloheptanediyl, bicyclo[2.2.2]octanediyl, phendiyl, pyrazoldiyl, imidazoldiyl, tetrazoldiyl, pyrimidindiyl, 5,6,7,8,9,10-hexahydrocycloocta[d]pyridazindiyl, azetidindiyl, 1,3-dioxandiyl, pyrrolidindiyl, piperazindiyl, piperidindiyl, or 3,9-diazaspiro[5.5]undecanediyl, each optionally substituted with one or more substituents Q. In certain embodiments, each Rⁿ is independently methanediyl, ethane-1,2-diyl, propane-1,3-diyl, butane-1,4-diyl, pentane-1,5-diyl, hexane-1,6-diyl, ethyne-1,2-diyl, propyne-1,3-diyl, 1-pentyne-1,5-diyl, cyclobutane-1,3-diyl, cyclopentane-1,3-diyl, cyclohexane-1,3-diyl, cyclohexane-1,4-diyl, cycloheptane-1,3-diyl, cycloheptane-1,4-diyl, bicyclo[2.2.2]octane-1,4-diyl, phen-1,3-diyl, phen-1,4-diyl, pyrazol-1,3-diyl, pyrazol-1,4-diyl, imidazol-1,4-diyl, 1,2,3-triazol-1,4-diyl, pyrimidin-2,4-diyl, pyrimidin-2,5-diyl, 5,6,7,8,9,10-hexahydrocycloocta[d]-pyridazin-1,7-diyl, pyrazolidin-1,3-diyl, pyrazolidin-1,4-diyl, azetidin-1,3-diyl, 1,3-dioxan-2,5-diyl, pyrrolidin-1,3-diyl, piperazin-1,4-diyl, piperidin-1,3-diyl, piperidin-1,4-diyl, or 3,9-diazaspiro[5.5]-undecane-3,9-diyl, each optionally substituted with one or more substituents Q. In certain embodiments, each Rⁿ is independently methanediyl, ethane-1,2-diyl, propane-1,3-diyl, butane-1,4-diyl, pentane-1,5-diyl, hexane-1,6-diyl, azetidin-1,3-diyl, piperidin-1,4-diyl, or piperazin-1,4-diyl.

In certain embodiments, each Rⁿ is independently methanediyl, ethane-1,2-diyl, propane-1,3-diyl, butane-1,4-diyl, pentane-1,5-diyl, hexane-1,6-diyl, phen-1,3-diyl, phen-1,4-diyl, azetidin-1,3-diyl, piperidin-1,4-diyl, or piperazin-1,4-diyl, each optionally substituted with one or more substituents Q. In certain embodiments, each Rⁿ is independently methanediyl, ethane-1,2-diyl, phen-1,4-diyl, piperidin-1,4-diyl, or piperazin-1,4-diyl, each optionally substituted with one, two, three, or four fluoro.

In certain embodiments, each Zⁿ is independently a bond, —C(O)—, —C(O)O—, —C(O)NR^{1b}—, —O—, —OC(O)NR^{1b}—, —NR^{1b}—, —NR^{1a}C(O)NR^{1b}—, —NR^{1a}S(O)NR^{1b}—, —NR^{1a}S(O)₂NR^{1b}—, —S—, —S(O)—, —S(O)₂—, —S(O)NR^{1b}—, or —S(O)₂NR^{1b}—; wherein each R^{1a} and R^{1b} is as defined herein. In certain embodiments, each Zⁿ is independently a bond, —C(O)—, —C(O)O—, —C(O)NR^{1b}—, —O—, —OC(O)NR^{1b}—, —NR^{1b}—, or —S(O)₂—; wherein each R^{1b} is as defined herein. In certain embodiments, each Zⁿ is independently a bond, —C(O)—, —C(O)O—, —C(O)NH—, —O—, —OC(O)NH—, —NH—, —N(CH₃)—, or —S(O)₂—. In certain embodiments, each Zⁿ is independently a bond, —C(O)—, —C(O)NR^{1b}—, —O—, or —NR^{1b}—, wherein each R^{1b} is as defined herein. In certain embodiments, each Zⁿ is independently a bond, —C(O)—, —C(O)NH—, —O—, or —NH—.

In certain embodiments, z is an integer of 1. In certain embodiments, z is an integer of 2. In certain embodiments, z is an integer of 3. In certain embodiments, z is an integer of 4. In certain embodiments, z is an integer of 5.

In certain embodiments, each Rⁿ is independently C₁₋₆ alkylene or heterocyclylene, each optionally substituted with one or more substituents Q; each Zⁿ is independently a bond, —C(O)—, —C(O)NR^{1b}—, —O—, or —NR^{1b}—, wherein each R^{1b} is as defined herein; and z is an integer of 1, 2, 3, 4, or 5. In certain embodiments, each Rⁿ is independently methanediyl, ethanediyl, propanediyl, butanediyl, pentanediyl, hexanediyl, azetidindiyl, piperidindiyl, or piperazindiyl; each Zⁿ is independently a bond, —C(O)—, —C(O)NH—, —O—, or —NH—; and z is an integer of 1, 2, 3, 4, or 5. In certain embodiments, each Rⁿ is independently methanediyl, ethane-1,2-diyl, propane-1,3-diyl, butane-1,4-diyl, pentane-1,5-diyl, hexane-1,6-diyl, azetidin-1,3-diyl, piperidin-1,4-diyl, or piperazin-1,4-diyl; each Zⁿ is independently a bond, —C(O)—, —C(O)NH—, —O—, or —NH—; and z is an integer of 1, 2, 3, 4, or 5.

In certain embodiments, L is:

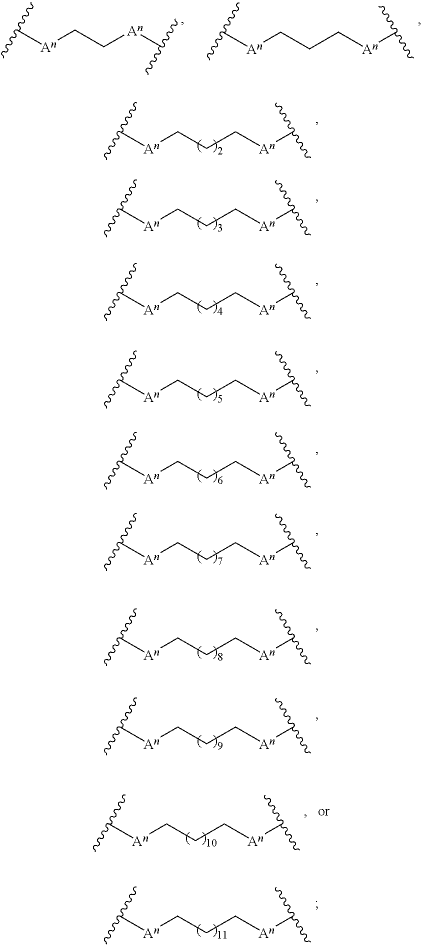

wherein each Aⁿ is independently a bond, —C(O)—, —C(O)NH—, —O—, —NH—, —N(CH₃)—, or —S(O)₂—.

In certain embodiments, L is:

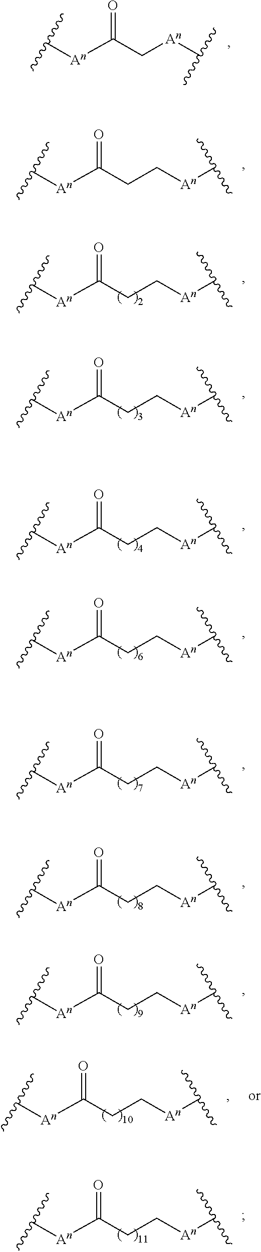

wherein each Aⁿ is independently a bond, —C(O)—, —C(O)NH—, —O—, —NH—, —N(CH₃)—, or —S(O)₂—.

In certain embodiments, L is:

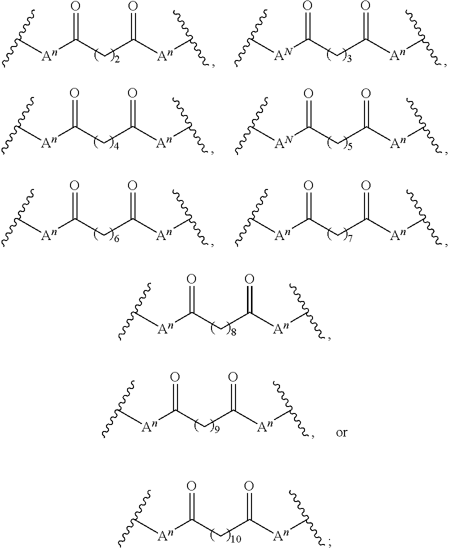

wherein each Aⁿ is independently a bond, —C(O)—, —C(O)NH—, —O—, —NH—, —N(CH₃)—, or —S(O)₂—.

In certain embodiments, L is:

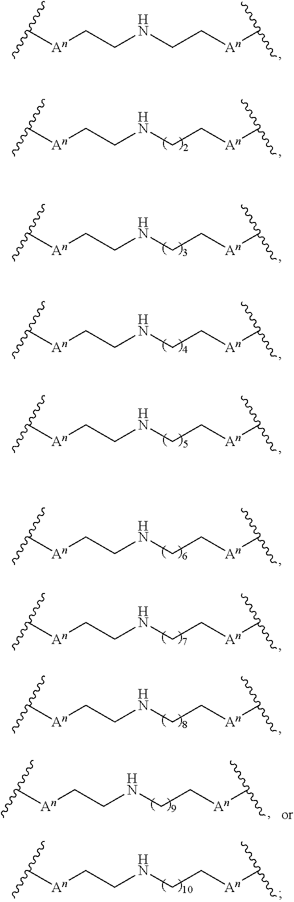

wherein each Aⁿ is independently a bond, —C(O)—, —C(O)NH—, —O—, —NH—, —N(CH₃)—, or —S(O)₂—; and wherein each amino (NH) group is optionally substituted with methyl.

In certain embodiments, L is:

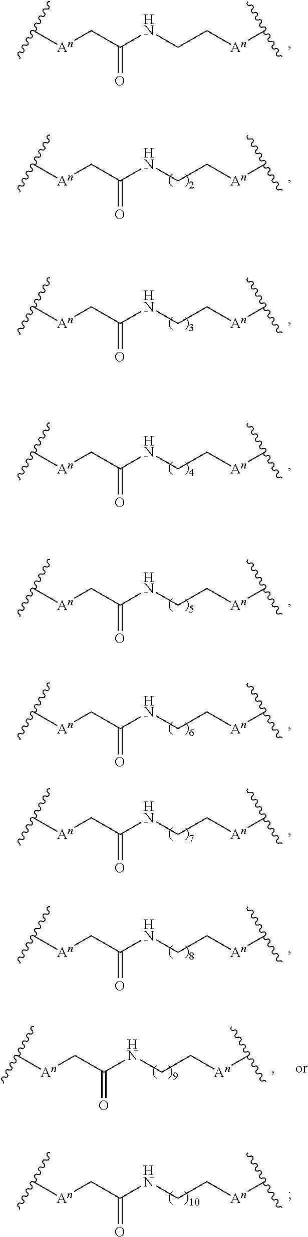

wherein each Aⁿ is independently a bond, —C(O)—, —C(O)NH—, —O—, —NH—, —N(CH₃)—, or —S(O)₂—; and wherein each amino (NH) group is optionally substituted with methyl.

In certain embodiments, L is:

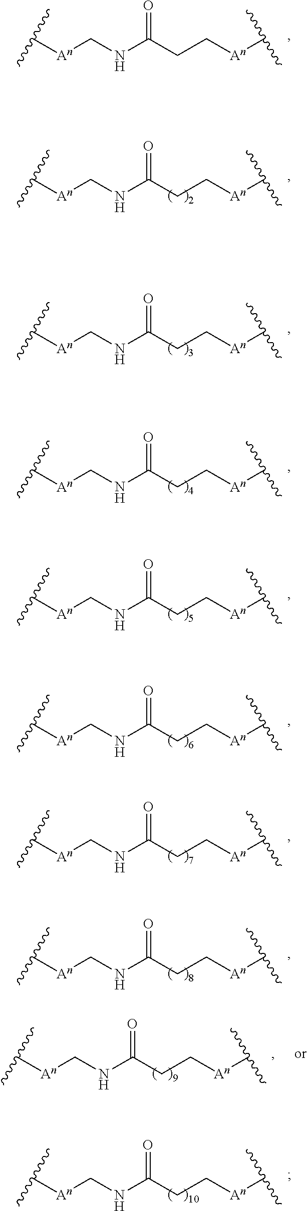

wherein each Aⁿ is independently a bond, —C(O)—, —C(O)NH—, —O—, —NH—, —N(CH₃)—, or —S(O)₂—; and wherein each amino group (NH) is optionally substituted with methyl.

In certain embodiments, L is:

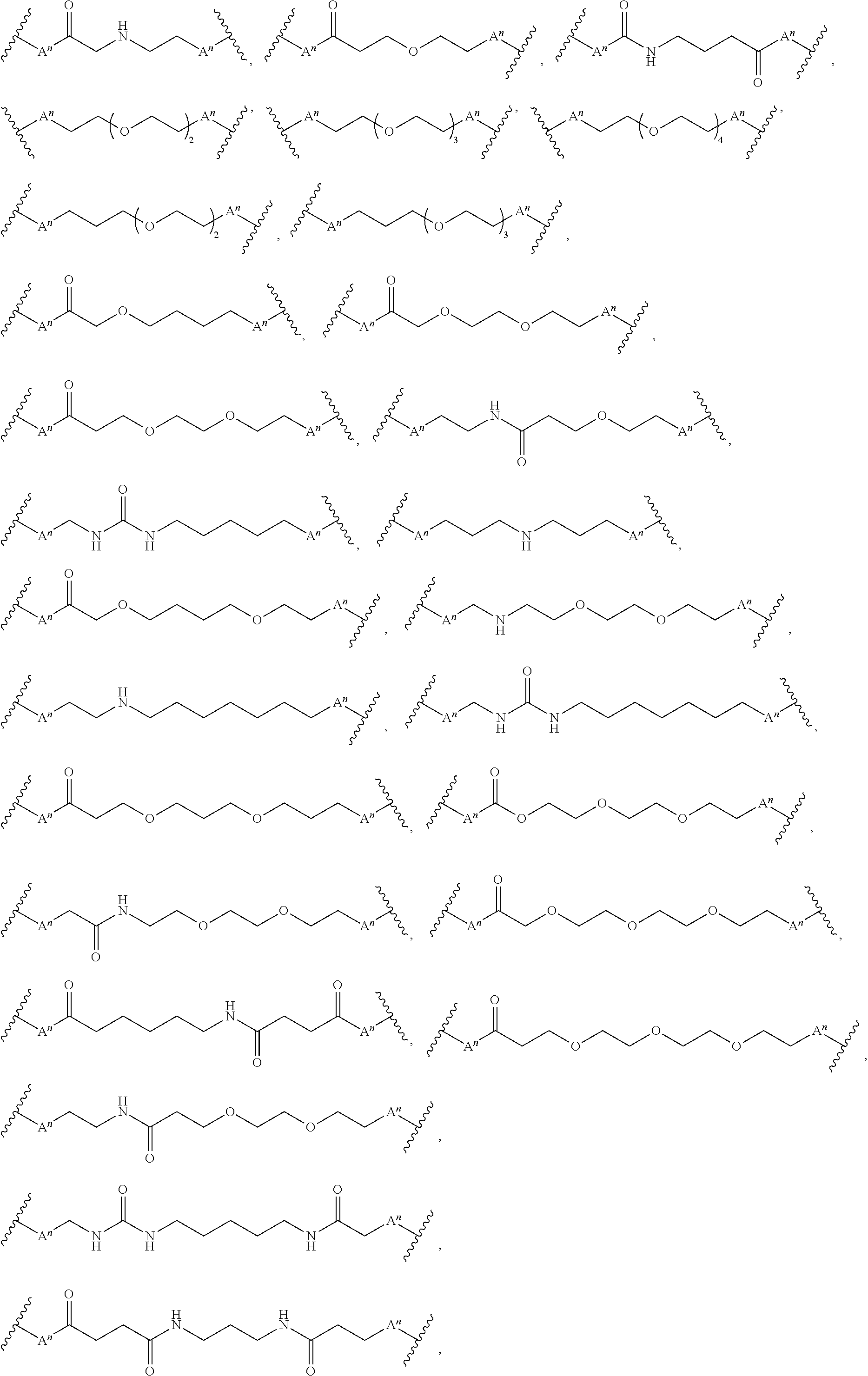

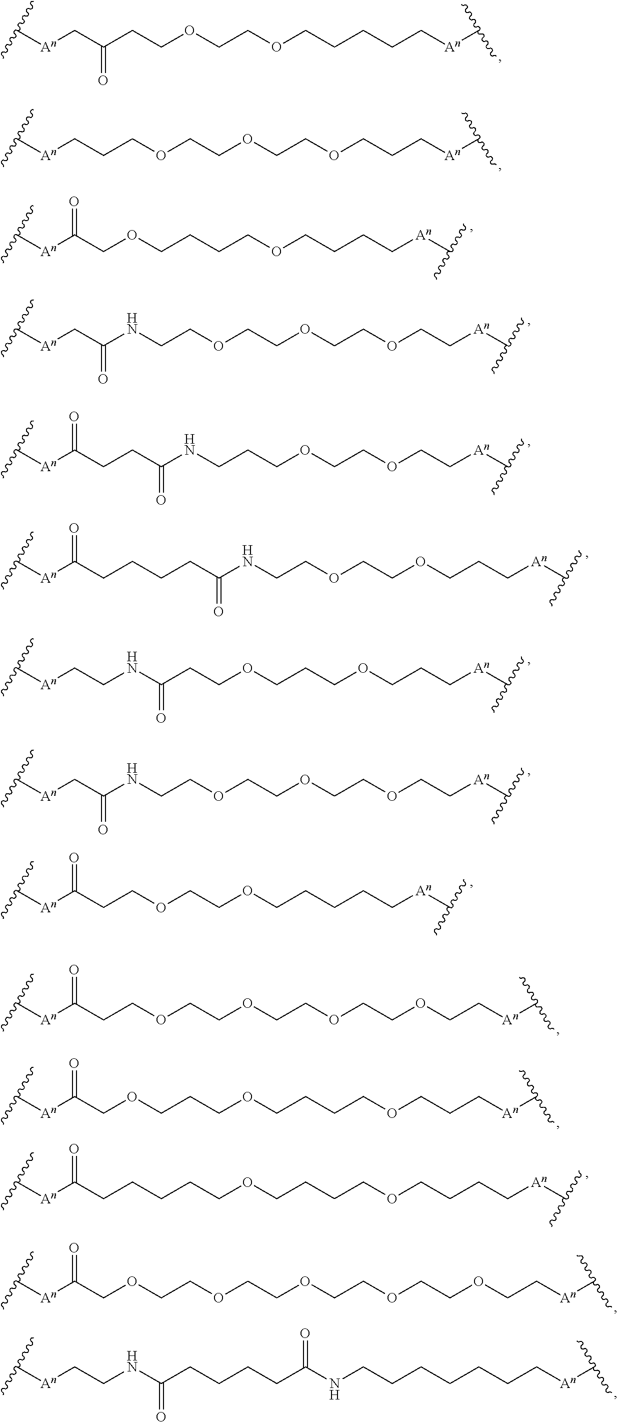

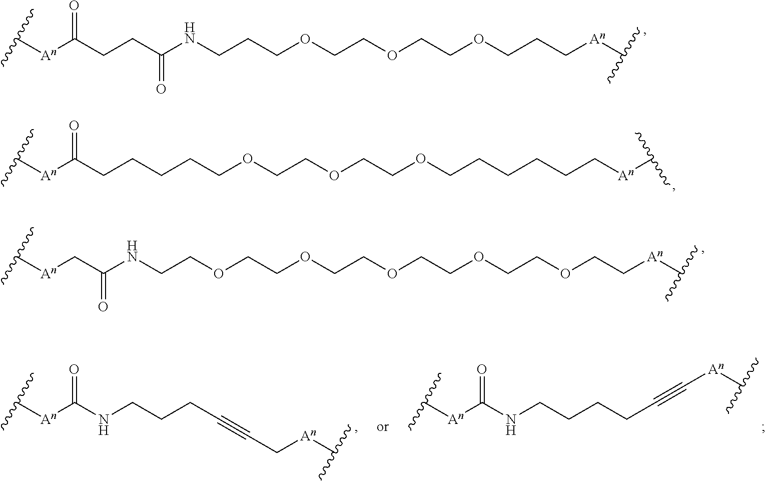

wherein each Aⁿ is independently a bond, —C(O)—, —C(O)NH—, —O—, —NH—, —N(CH₃)—, or —S(O)₂—; and wherein each amino (NH) group is optionally substituted with methyl.

In certain embodiments, L is:

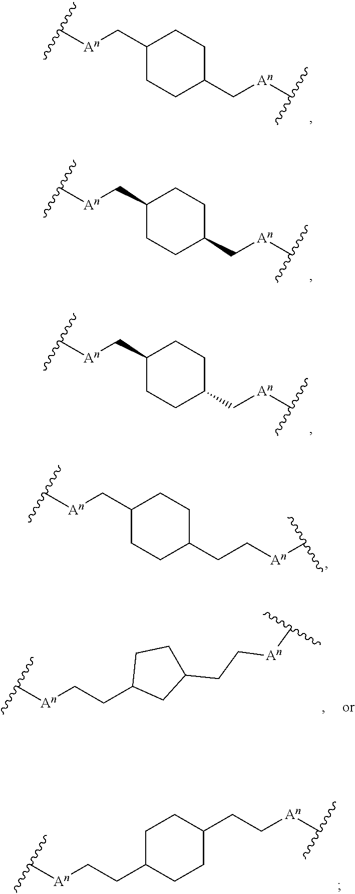

wherein each Aⁿ is independently a bond, —C(O)—, —C(O)NH—, —O—, —NH—, —N(CH₃)—, or —S(O)₂—.

In certain embodiments, L is:

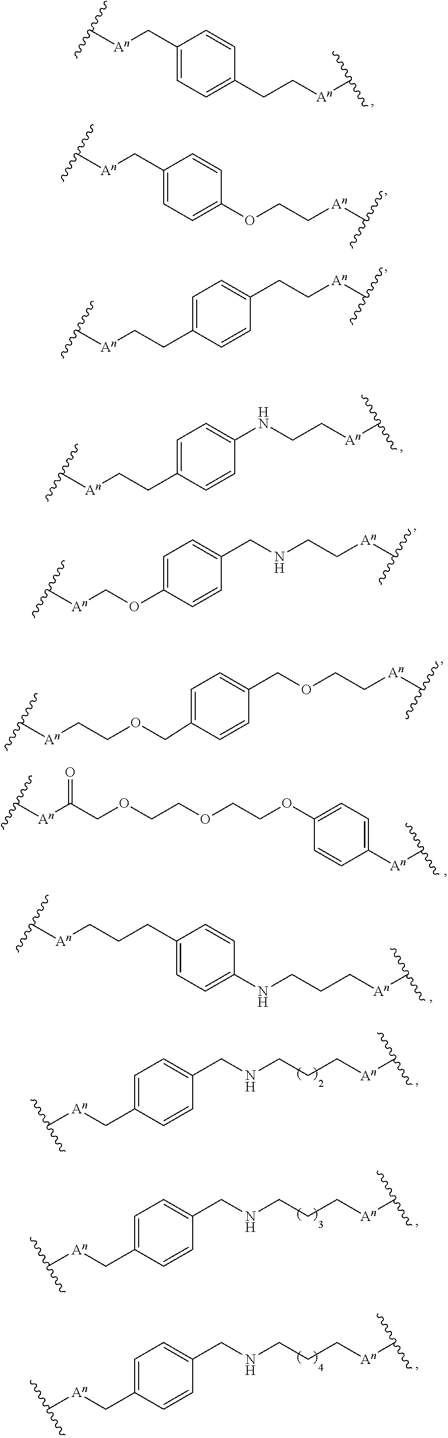

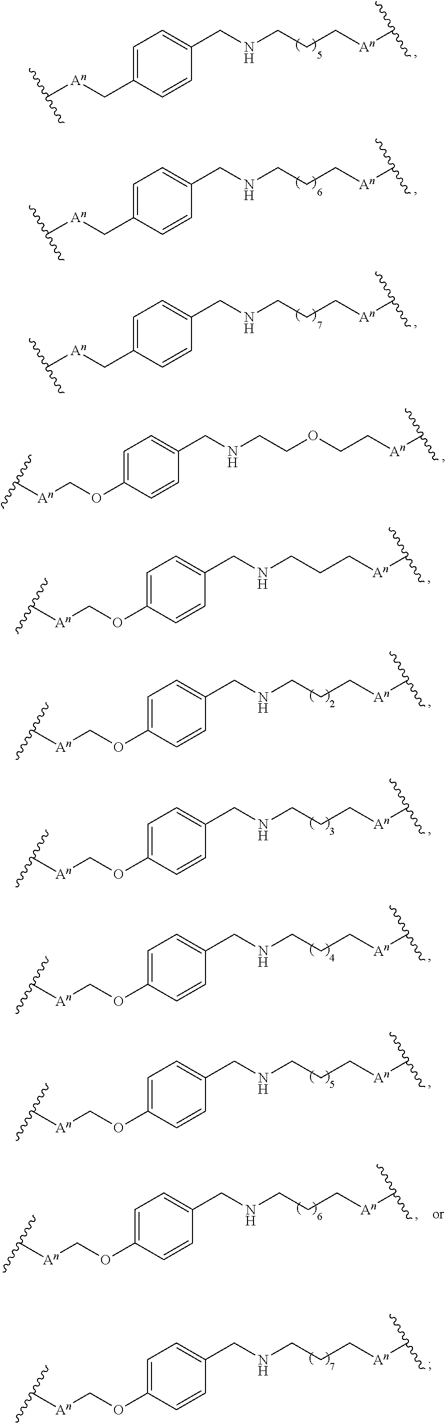

wherein each Aⁿ is independently a bond, —C(O)—, —C(O)NH—, —O—, —NH—, —N(CH₃)—, or —S(O)₂—; and wherein each amino group (NH) is optionally substituted with methyl.

In certain embodiments, L is:

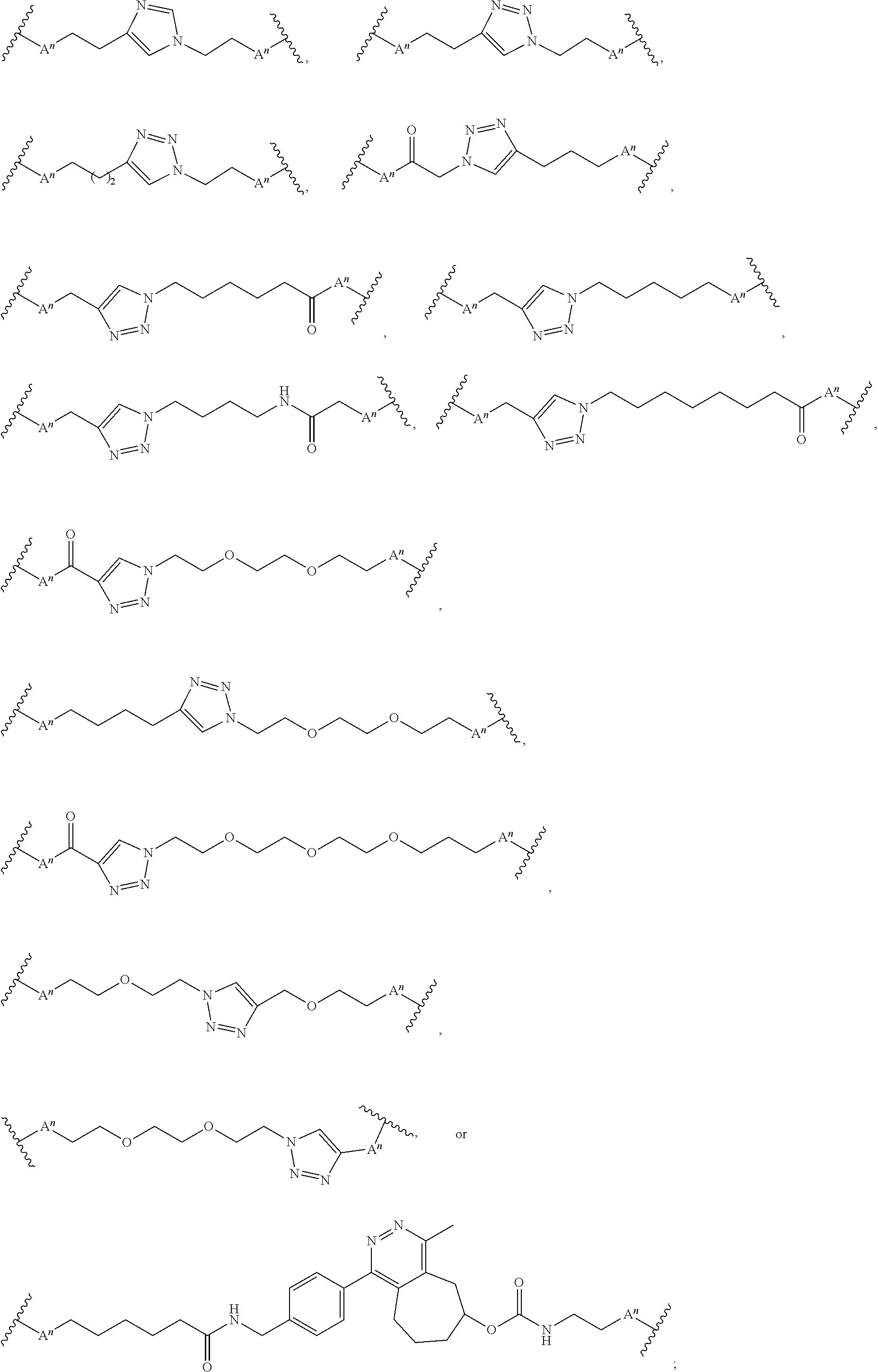

wherein each Aⁿ is independently a bond, —C(O)—, —C(O)NH—, —O—, —NH—, —N(CH₃)—, or —S(O)₂—; and wherein each amino group (NH) is optionally substituted with methyl.

In certain embodiments, L is:

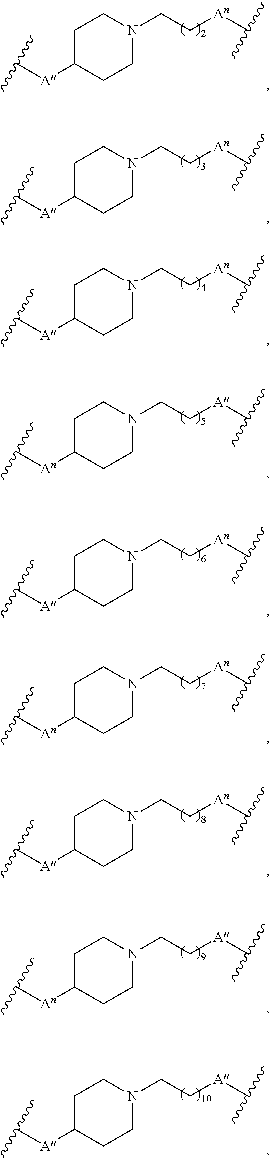

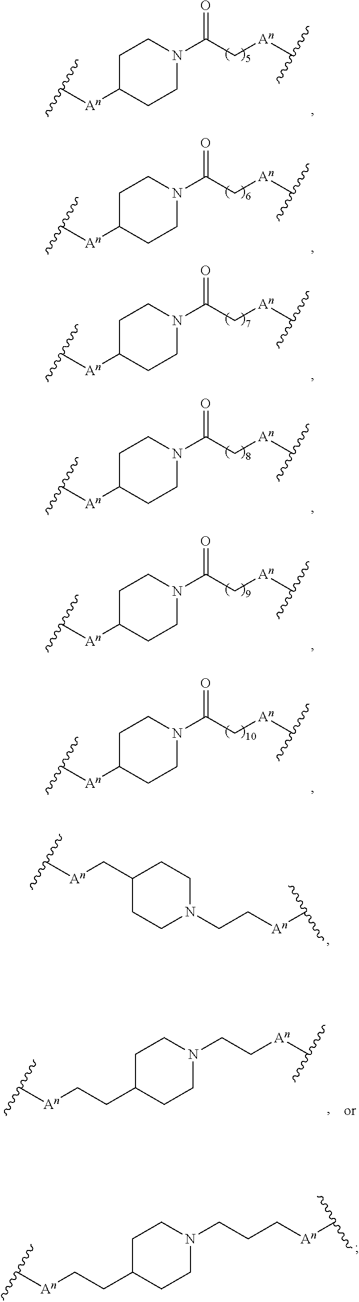

wherein each Aⁿ is independently a bond, —C(O)—, —C(O)NH—, —O—, —NH—, —N(CH₃)—, or —S(O)₂—.

In certain embodiments, L is:

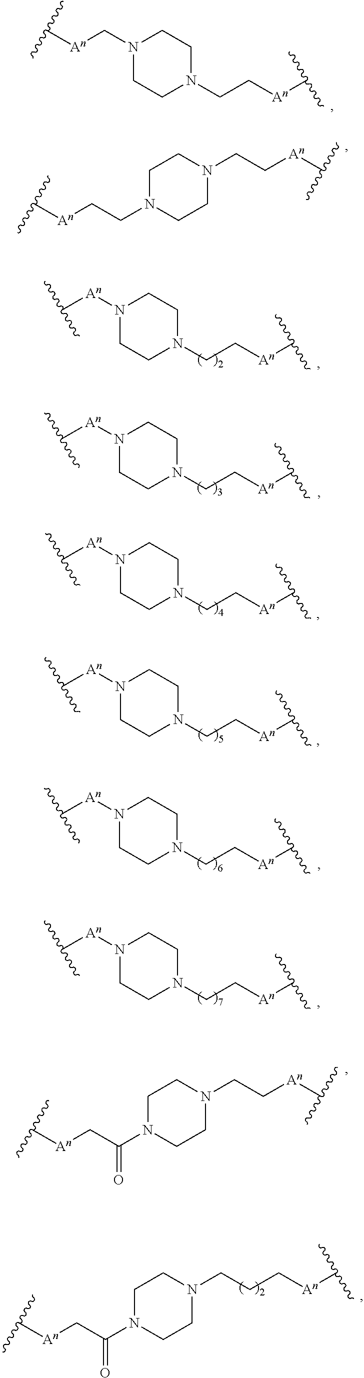

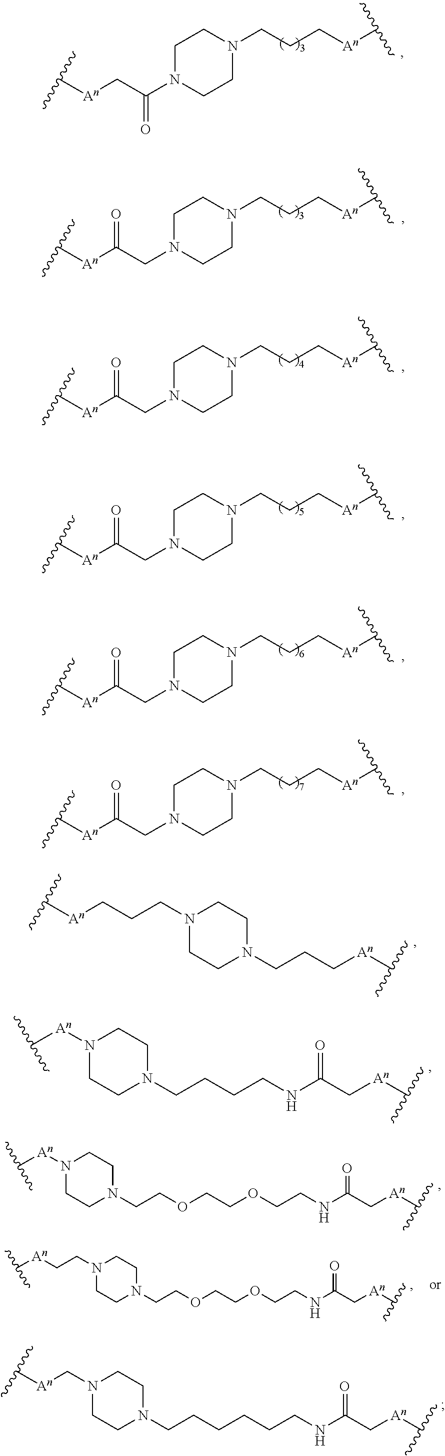

wherein each Aⁿ is independently a bond, —C(O)—, —C(O)NH—, —O—, —NH—, —N(CH₃)—, or —S(O)₂—; and wherein each amino group (NH) is optionally substituted with methyl.

In certain embodiments, L is:

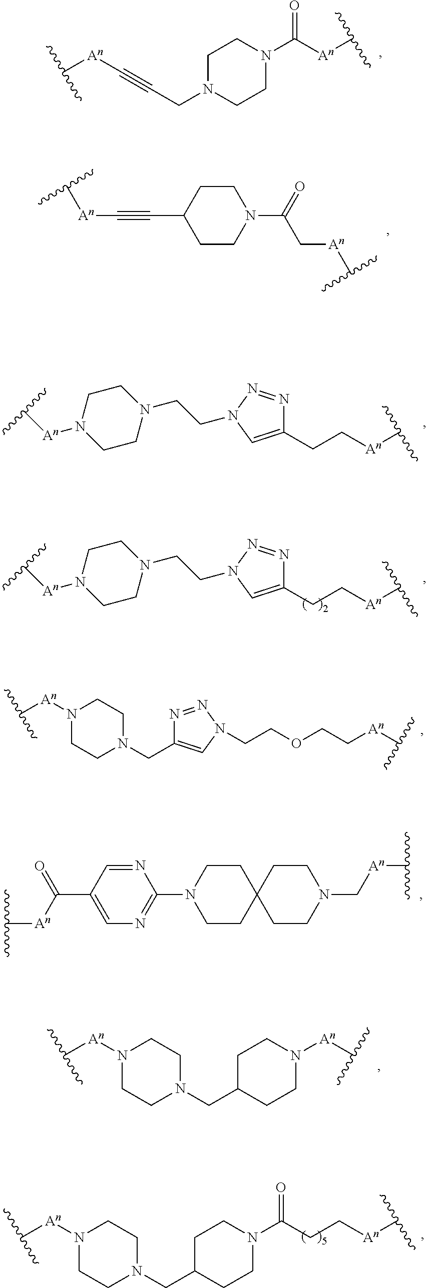

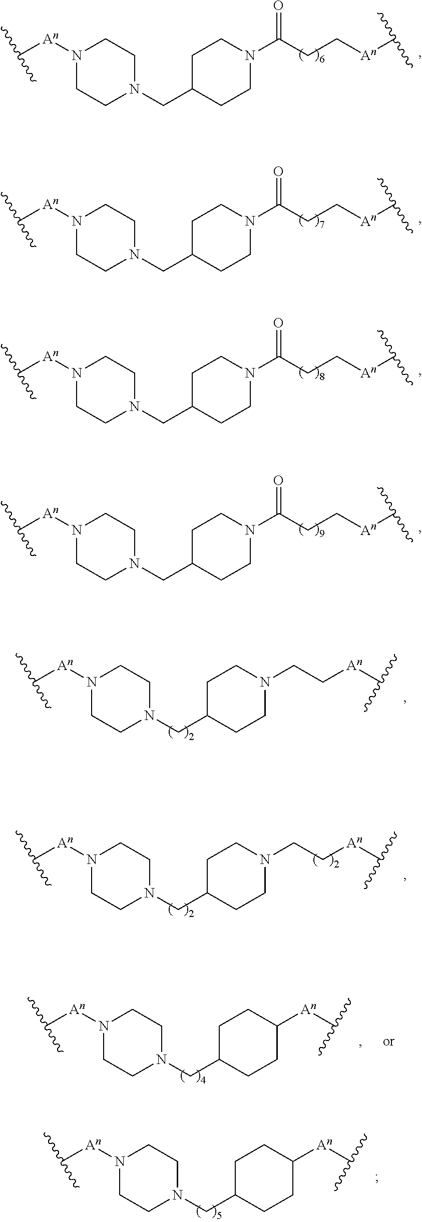

wherein each Aⁿ is independently a bond, —C(O)—, —C(O)NH—, —O—, —NH—, —N(CH₃)—, or —S(O)₂—; and wherein each amino group is optionally substituted with methyl.

In certain embodiments, L is:

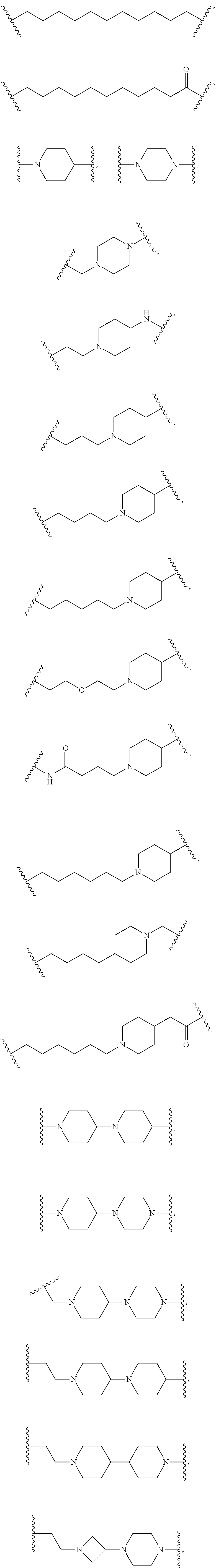

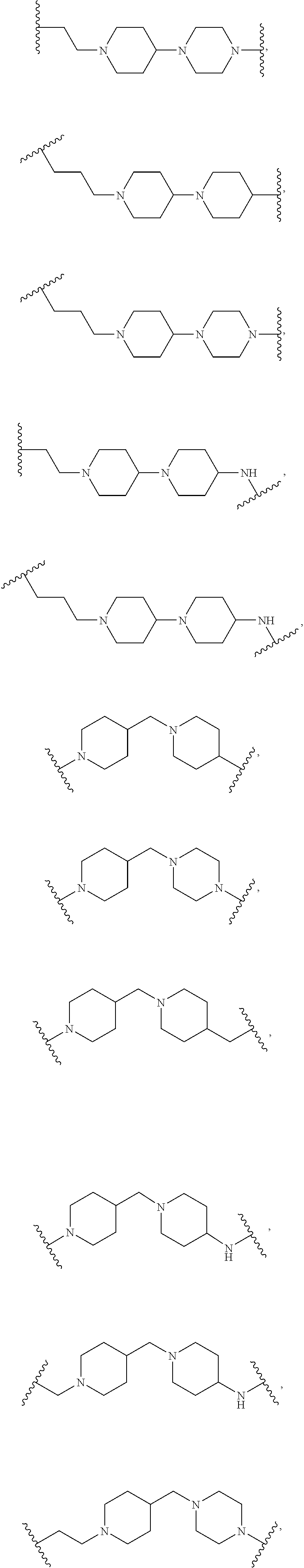

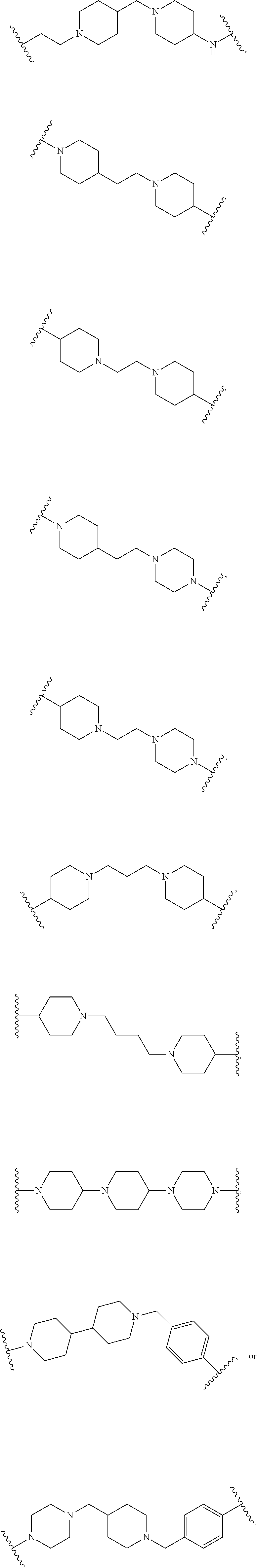

In certain embodiments, L is:

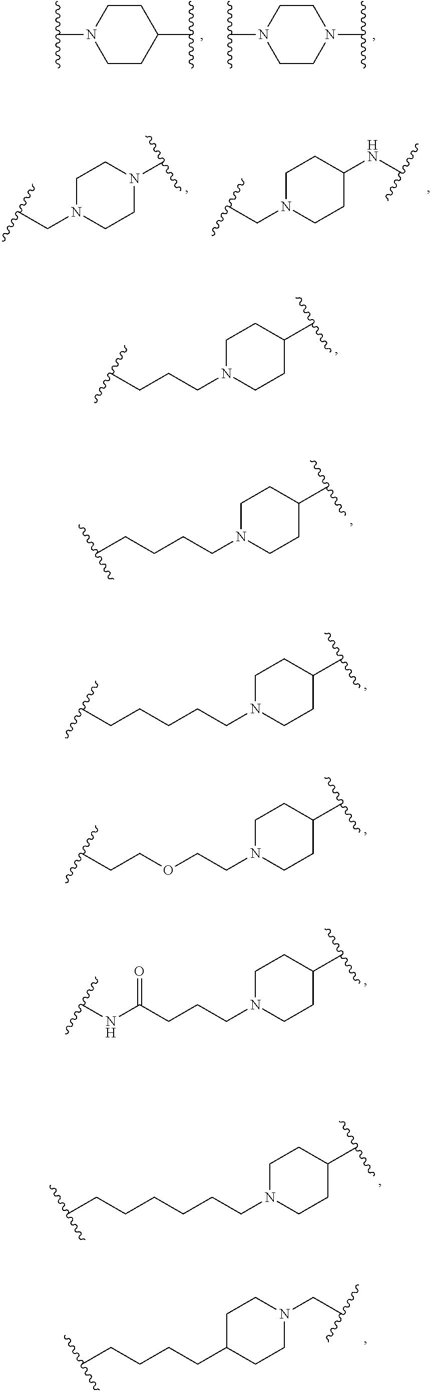

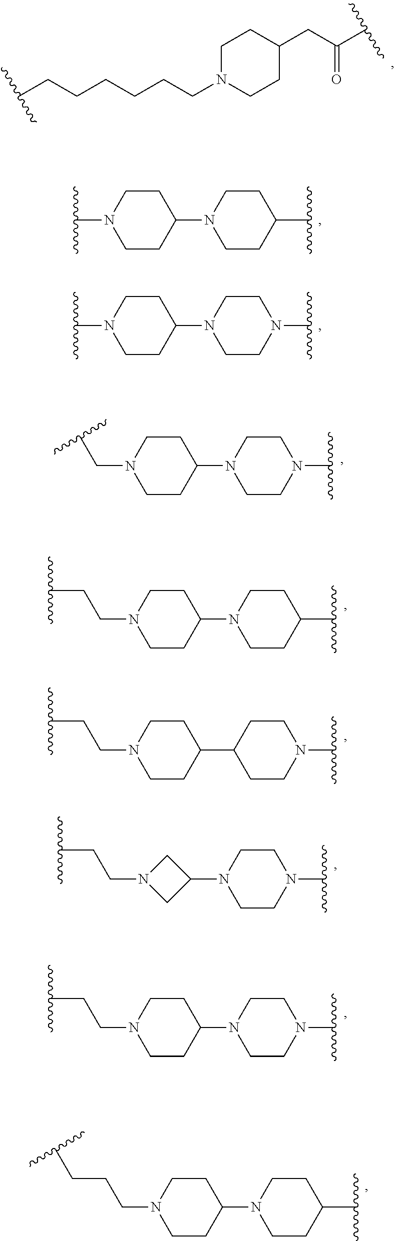

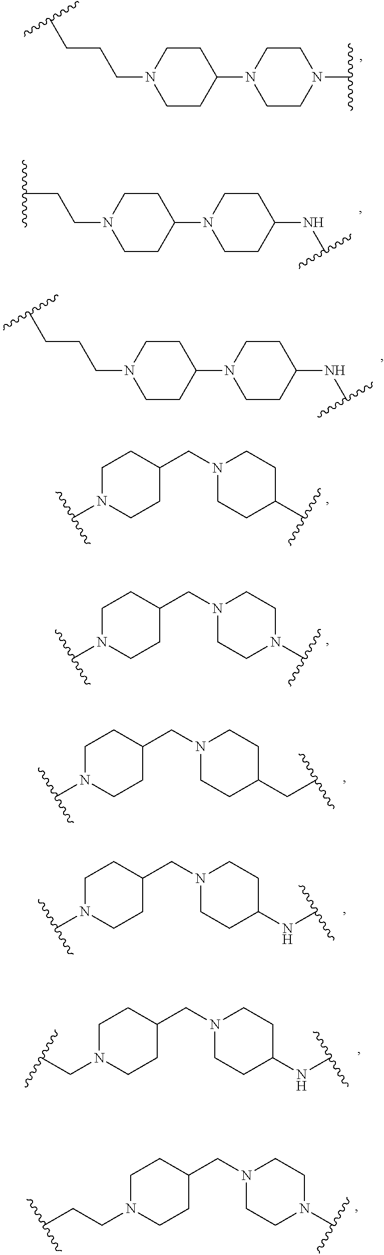

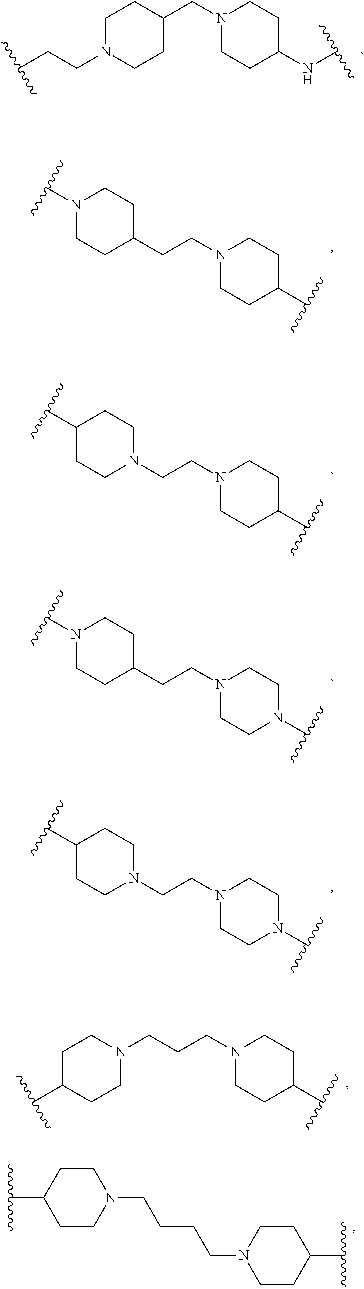

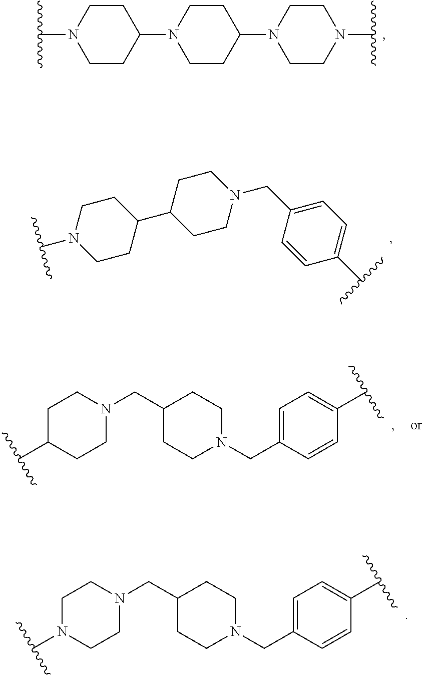 [SMILES: *N1CCC(N2CCC(N3CCN(*)CC3)CC2)CC1 ; *c1ccc(CN2CCC(C3CCN(*)CC3)CC2)cc1 ; *c1ccc(CN2CCC(CN3CCC(*)CC3)CC2)cc1 ; *c1ccc(CN2CCC(CN3CCN(*)CC3)CC2)cc1]

In certain embodiments, L is:

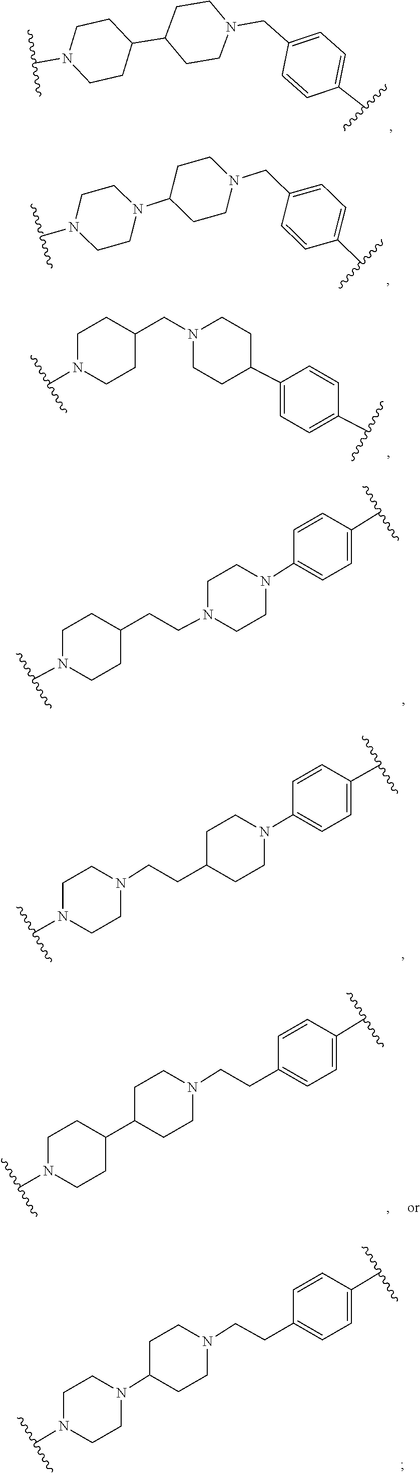

each of which is optionally substituted with one, two, three, or four halo, in one embodiment, optionally substituted with one, two, three, or four fluoro.

In certain embodiments, L is:

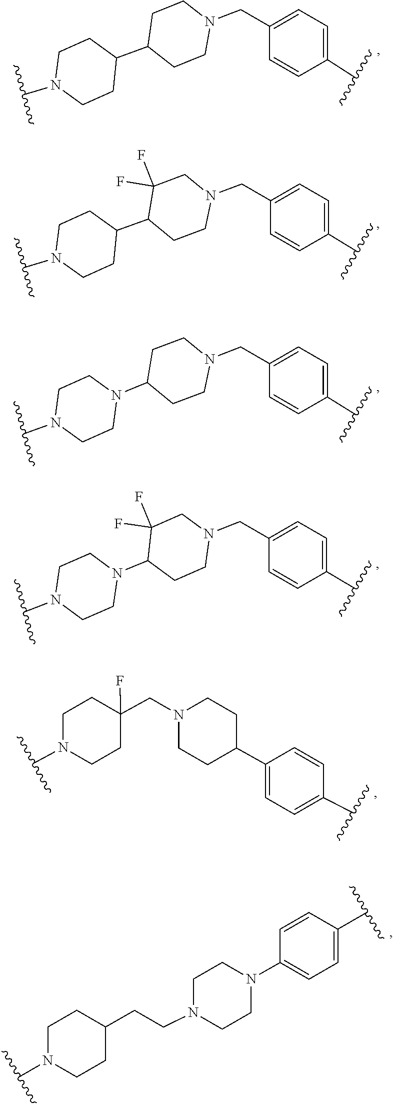 [SMILES: *c1ccc(CN2CCC(C3CCN(*)CC3)CC2)cc1 ; *c1ccc(CN2CCC(C3CCN(*)CC3)C(F)(F)C2)cc1 ; *c1ccc(CN2CCC(N3CCN(*)CC3)CC2)cc1 ; *c1ccc(CN2CCC(N3CCN(*)CC3)C(F)(F)C2)cc1 ; *c1ccc(C2CCN(CC3(F)CCN(*)CC3)CC2)cc1 ; *c1ccc(N2CCN(CCC3CCN(*)CC3)CC2)cc1]

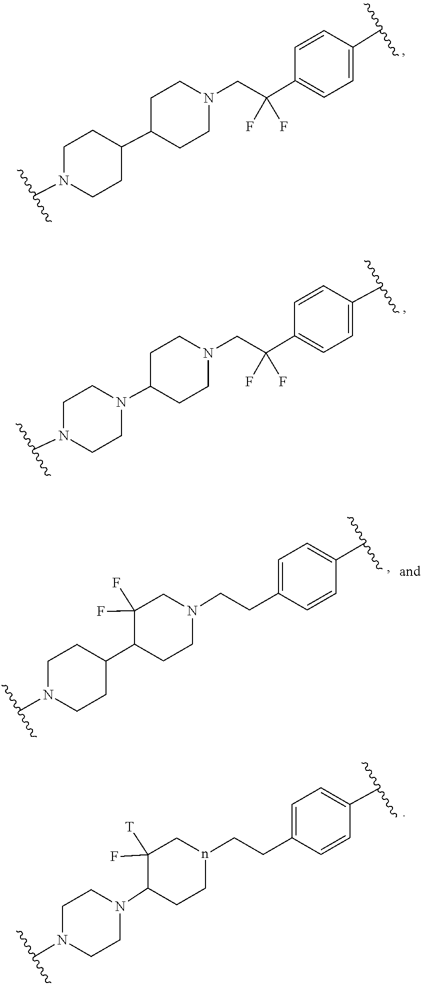

In certain embodiments, the moiety “L-A^{e}” has the structure of:

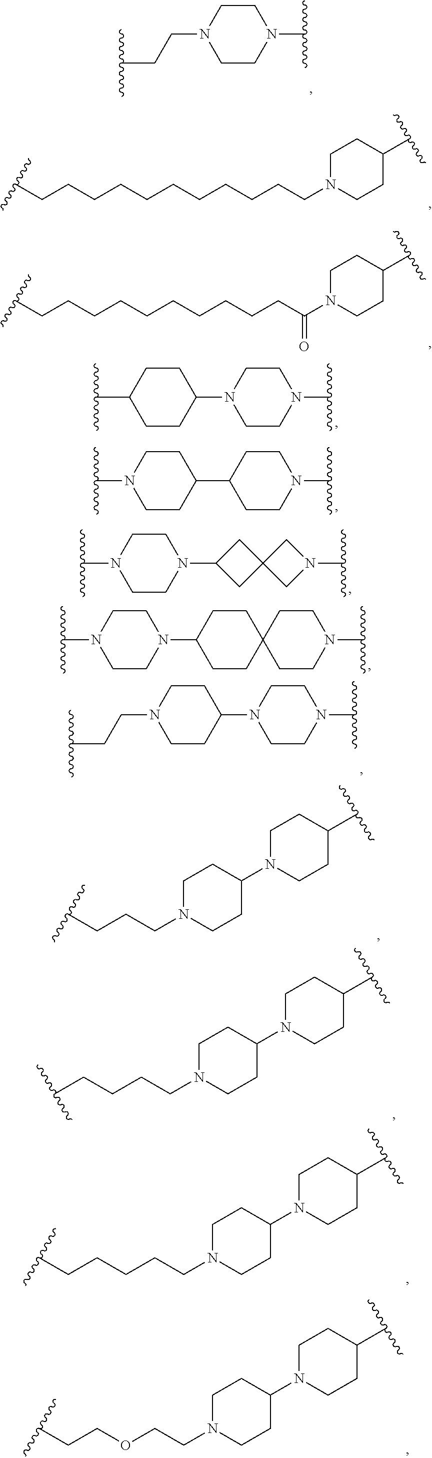

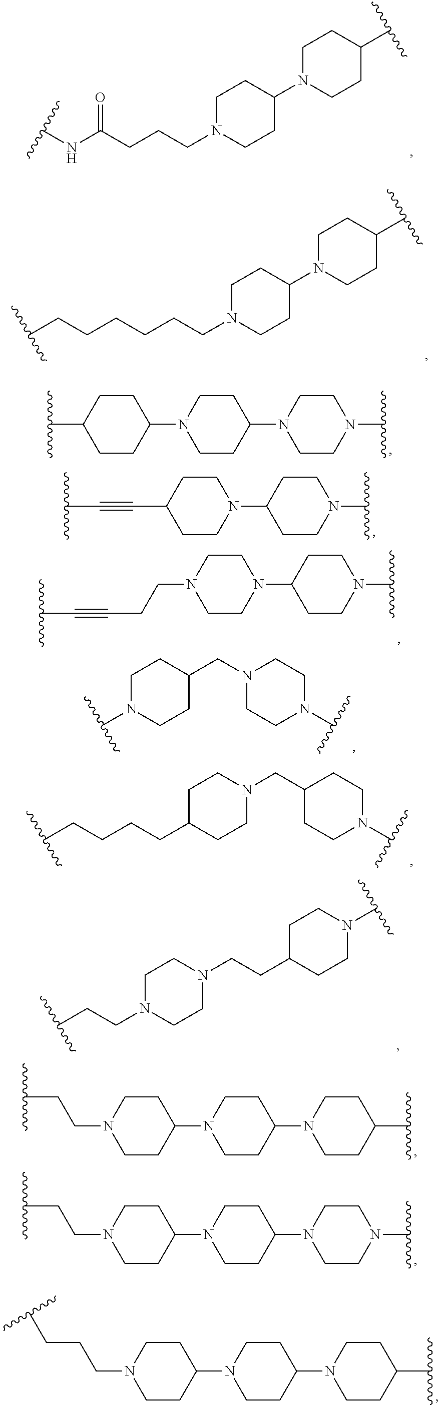

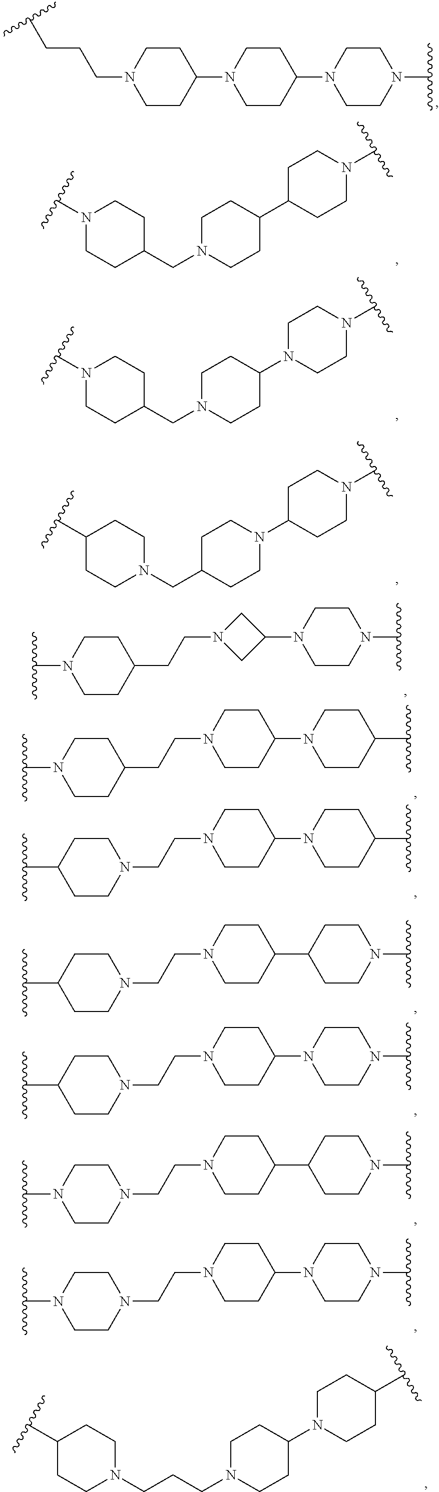

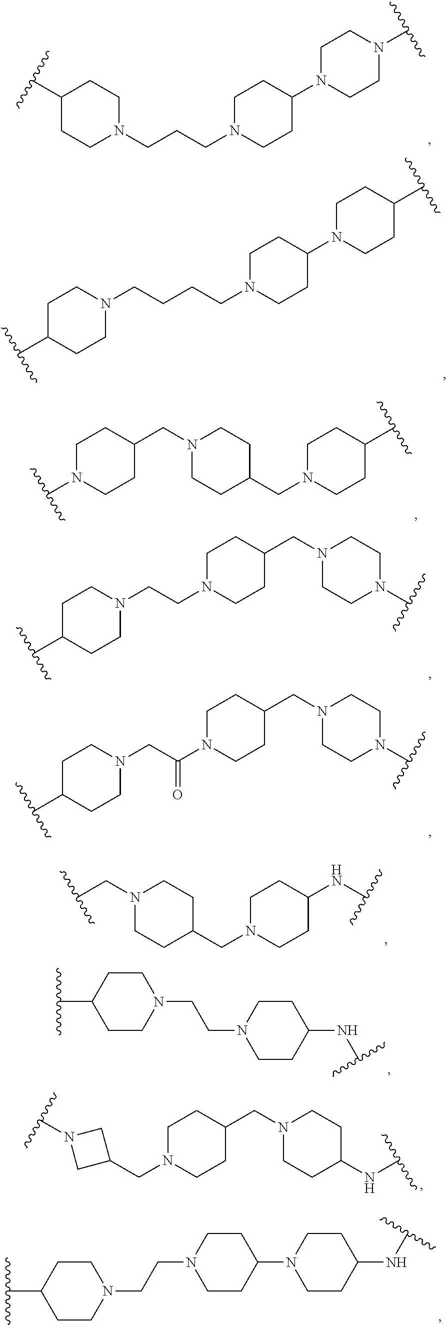

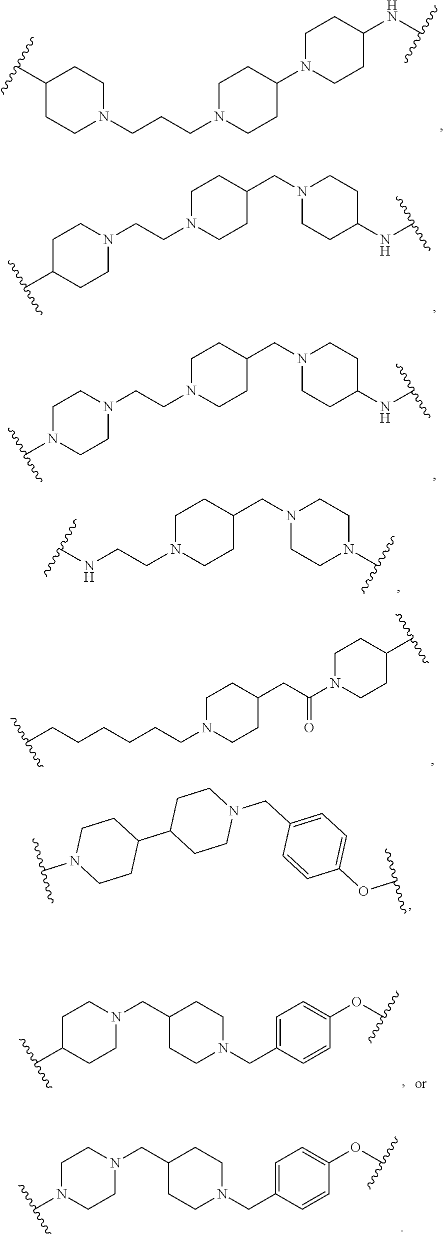

In certain embodiments, the moiety “L-A^{e}” has the structure of:

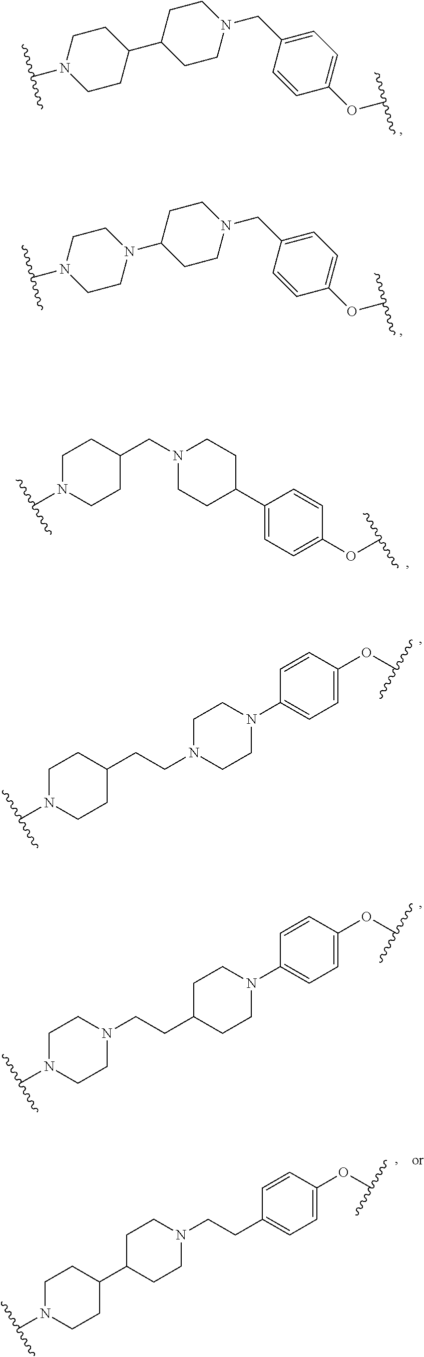 [SMILES: *Oc1ccc(CN2CCC(C3CCN(*)CC3)CC2)cc1 ; *Oc1ccc(CN2CCC(N3CCN(*)CC3)CC2)cc1 ; *Oc1ccc(C2CCN(CC3CCN(*)CC3)CC2)cc1 ; *Oc1ccc(N2CCN(CCC3CCN(*)CC3)CC2)cc1 ; *Oc1ccc(N2CCC(CCN3CCN(*)CC3)CC2)cc1 ; *Oc1ccc(CCN2CCC(C3CCN(*)CC3)CC2)cc1]

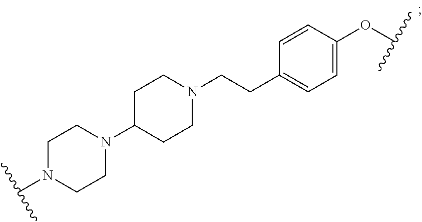 [SMILES: *Oc1ccc(CCN2CCC(N3CCN(*)CC3)CC2)cc1]

each of which is optionally substituted with one, two, three, or four halo, in one embodiment, optionally substituted with one, two, three, or four fluoro.

In certain embodiments, the moiety “L-A^{e}” has the structure of:

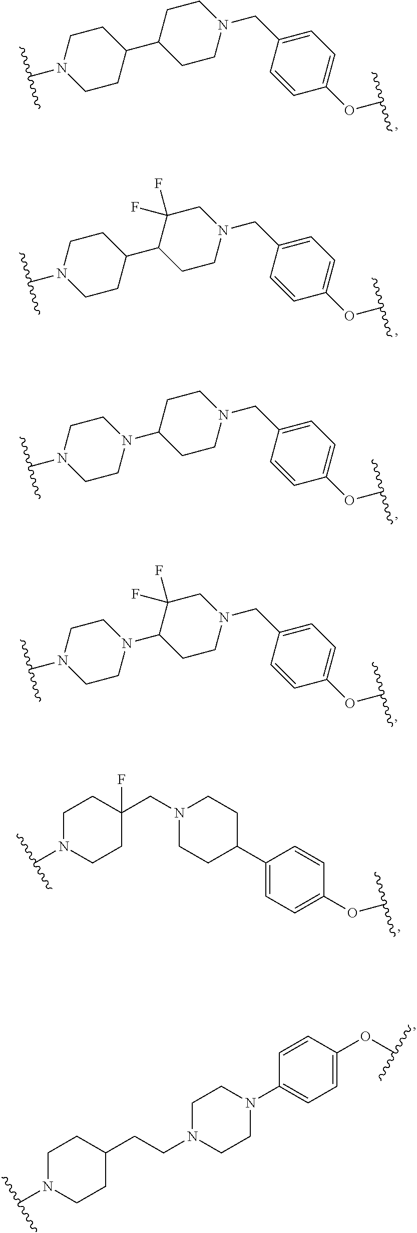 [SMILES: *Oc1ccc(CN2CCC(C3CCN(*)CC3)CC2)cc1 ; *Oc1ccc(CN2CCC(C3CCN(*)CC3)C(F)(F)C2)cc1 ; *Oc1ccc(CN2CCC(N3CCN(*)CC3)CC2)cc1 ; *Oc1ccc(CN2CCC(N3CCN(*)CC3)C(F)(F)C2)cc1 ; *Oc1ccc(C2CCN(CC3(F)CCN(*)CC3)CC2)cc1 ; *Oc1ccc(N2CCN(CCC3CCN(*)CC3)CC2)cc1]

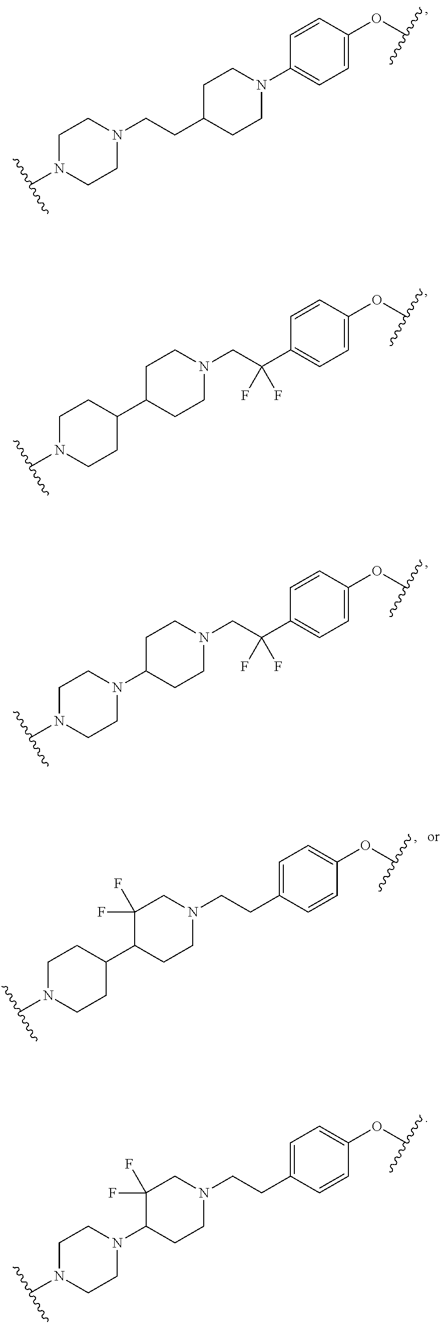 [SMILES: *Oc1ccc(N2CCC(CCN3CCN(*)CC3)CC2)cc1 ; *Oc1ccc(C(F)(F)CN2CCC(C3CCN(*)CC3)CC2)cc1 ; *Oc1ccc(C(F)(F)CN2CCC(N3CCN(*)CC3)CC2)cc1 ; *Oc1ccc(CCN2CCC(C3CCN(*)CC3)C(F)(F)C2)cc1 ; *Oc1ccc(CCN2CCC(N3CCN(*)CC3)C(F)(F)C2)cc1]

All combinations of the embodiments provided herein for the groups, R¹, R², R⁴, R^{2a}, R^{3a}, R^{3b}, R^{3c}, R^{3d}, R^{3e}, R^{4a}, R^{e}, R^{e1}, R^{e2}, R^{e3}, R^{e4}, R^{e5}, R^{e6}, A, A^{e}, L, X, X^{e}, Y, Y^{e}, Z^{e}, a, m, and n, in the formulae described herein, including Formulae A, (I), (IA) to (XLVIIA), (IB) to (XVB), (IC) to (XXIXC), and (ID) to (XXID), are within the scope of this disclosure.

In one embodiment, provided herein is:
3-(4-(1′-(5-(2-((S)-1-(3-ethoxy-4-methoxyphenyl)-2-(methylsulfonyl)ethyl)-1-oxoisoindolin-5-yl)pentyl)-[1,4′-bipiperidin]-4-yl)phenyl)piperidine-2,6-dione A001;
5-(6-(4-(4-(2,6-dioxopiperidin-3-yl)phenyl)-[1,4′-bipiperidin]-1′-yl)hexyl)-2-((S)-1-(3-ethoxy-4-methoxyphenyl)-2-(methylsulfonyl)ethyl)isoindoline-1,3-dione A002;
3-(4-(1′-(5-(2-((S)-1-(3-ethoxy-4-methoxyphenyl)-2-(methylsulfonyl)ethyl)-3-oxoisoindolin-5-yl)pentyl)-[1,4′-bipiperidin]-4-yl)phenyl)piperidine-2,6-dione A003;
N-(6-(5-(4-(4-(2,6-dioxopiperidin-3-yl)phenyl)-[1,4′-bipiperidin]-1′-yl)pentyl)-2-((S)-1-(3-ethoxy-4-methoxyphenyl)-2-(methylsulfonyl)ethyl)-1,3-dioxoisoindolin-4-yl)-acetamide A004;
5-(5-(4-(3-(2,6-dioxopiperidin-3-yl)phenyl)-[1,4′-bipiperidin]-1′-yl)pentyl)-2-((S)-1-(3-ethoxy-4-methoxyphenyl)-2-(methylsulfonyl)ethyl)isoindoline-1,3-dione A005;
5-(2-(2-(4-(4-(2,6-dioxopiperidin-3-yl)phenyl)-[1,4′-bipiperidin]-1′-yl)ethoxy)-ethyl)-2-((S)-1-(3-ethoxy-4-methoxyphenyl)-2-(methylsulfonyl)ethyl)isoindoline-1,3-dione A006;
5-(4-(1-((1-(4-(2,6-dioxopiperidin-3-yl)phenyl)piperidin-4-yl)methyl)piperidin-4-yl)butyl)-2-((S)-1-(3-ethoxy-4-methoxyphenyl)-2-(methylsulfonyl)ethyl)isoindoline-1,3-dione A007;
4-(3-(4-(4-(2,6-dioxopiperidin-3-yl)phenyl)-[1,4′:1′,4″-terpiperidin]-1″-propyl)-2-((S)-1-(3-ethoxy-4-methoxyphenyl)-2-(methylsulfonyl)ethyl)isoindoline-1,3-dione A008;
5-(3-(4-(4-(2,6-dioxopiperidin-3-yl)phenyl)-[1,4′:1′,4″-terpiperidin]-1″-propyl)-2-((S)-1-(3-ethoxy-4-methoxyphenyl)-2-(methylsulfonyl)ethyl)isoindoline-1,3-dione A009;
4-(2-(4-(4-(2,6-dioxopiperidin-3-yl)phenyl)-[1,4′:1′,4″-terpiperidin]-1″ethyl)-2-((S)-1-(3-ethoxy-4-methoxyphenyl)-2-(methylsulfonyl)ethyl)isoindoline-1,3-dione A010;
5-(2-(4-(4-(2,6-dioxopiperidin-3-yl)phenyl)-[1,4′:1′,4″-terpiperidin]-1″ethyl)-2-((S)-1-(3-ethoxy-4-methoxyphenyl)-2-(methylsulfonyl)ethyl)isoindoline-1,3-dione A011;
5-(4-(1′-(3-(4-(2,6-dioxopiperidin-3-yl)phenyl)propyl)-[1,4′-bipiperidin]-4-yl)-piperazin-1-yl)-2-((S)-1-(3-ethoxy-4-methoxyphenyl)-2-(methylsulfonyl)ethyl)isoindoline-1,3-dione A012;
4-(4-(1′-(3-(4-(2,6-dioxopiperidin-3-yl)phenyl)propyl)-[1,4′-bipiperidin]-4-yl)-piperazin-1-yl)-2-((S)-1-(3-ethoxy-4-methoxyphenyl)-2-(methylsulfonyl)ethyl)isoindoline-1,3-dione A013;
4-(4-(1′-(4-(2,6-dioxopiperidin-3-yl)phenethyl)-[1,4′-bipiperidin]-4-yl)piperazin-1-yl)-2-((S)-1-(3-ethoxy-4-methoxyphenyl)-2-(methylsulfonyl)ethyl)isoindoline-1,3-dione A014;
5-(4-(1′-(4-(2,6-dioxopiperidin-3-yl)phenethyl)-[1,4′-bipiperidin]-4-yl)piperazin-1-yl)-2-((S)-1-(3-ethoxy-4-methoxyphenyl)-2-(methylsulfonyl)ethyl)isoindoline-1,3-dione A015;
4-((1′-(3-(4-(4-(2,6-dioxopiperidin-3-yl)phenyl)piperidin-1-yl)propyl)-[1,4′-bipiperidin]-4-yl)amino)-2-((S)-1-(3-ethoxy-4-methoxyphenyl)-2-(methylsulfonyl)ethyl)-isoindoline-1,3-dione A016;
5-((1′-(3-(4-(4-(2,6-dioxopiperidin-3-yl)phenyl)piperidin-1-yl)propyl)-[1,4′-bipiperidin]-4-yl)amino)-2-((S)-1-(3-ethoxy-4-methoxyphenyl)-2-(methylsulfonyl)ethyl)-isoindoline-1,3-dione A017;
4-((1′-(2-(4-(4-(2,6-dioxopiperidin-3-yl)phenyl)piperidin-1-yl)ethyl)-[1,4′-bipiperidin]-4-yl)amino)-2-((S)-1-(3-ethoxy-4-methoxyphenyl)-2-(methylsulfonyl)ethyl)-isoindoline-1,3-dione A018;
5-((1′-(2-(4-(4-(2,6-dioxopiperidin-3-yl)phenyl)piperidin-1-yl)ethyl)-[1,4′-bipiperidin]-4-yl)amino)-2-((S)-1-(3-ethoxy-4-methoxyphenyl)-2-(methylsulfonyl)ethyl)-isoindoline-1,3-dione A019;
4-(4-(1-(3-(4-(4-(2,6-dioxopiperidin-3-yl)phenyl)piperidin-1-yl)propyl)piperidin-4-yl)piperazin-1-yl)-2-((S)-1-(3-ethoxy-4-methoxyphenyl)-2-(methylsulfonyl)ethyl)isoindoline-1,3-dione A020;
5-(4-(1-(3-(4-(4-(2,6-dioxopiperidin-3-yl)phenyl)piperidin-1-yl)propyl)piperidin-4-yl)piperazin-1-yl)-2-((S)-1-(3-ethoxy-4-methoxyphenyl)-2-(methylsulfonyl)ethyl)isoindoline-1,3-dione A021;
4-(4-(1-(2-(4-(4-(2,6-dioxopiperidin-3-yl)phenyl)piperidin-1-yl)ethyl)piperidin-4-yl)-piperazin-1-yl)-2-((S)-1-(3-ethoxy-4-methoxyphenyl)-2-(methylsulfonyl)ethyl)isoindoline-1,3-dione A022;
5-(4-(1-(2-(4-(4-(2,6-dioxopiperidin-3-yl)phenyl)piperidin-1-yl)ethyl)piperidin-4-yl)piperazin-1-yl)-2-((S)-1-(3-ethoxy-4-methoxyphenyl)-2-(methylsulfonyl)ethyl)isoindoline-1,3-dione A023;
5-(4-(1-((1-(4-(2,6-dioxopiperidin-3-yl)phenyl)piperidin-4-yl)methyl)piperidin-4-yl)piperazin-1-yl)-2-((S)-1-(3-ethoxy-4-methoxyphenyl)-2-(methylsulfonyl)ethyl)isoindoline-1,3-dione A024;
5-(4-(1-(2-(1-(4-(2,6-dioxopiperidin-3-yl)phenyl)piperidin-4-yl)ethyl)azetidin-3-yl)piperazin-1-yl)-2-((S)-1-(3-ethoxy-4-methoxyphenyl)-2-(methylsulfonyl)ethyl)isoindoline-1,3-dione A025;
4-(4-(4-(2,6-dioxopiperidin-3-yl)phenyl)-[1,4′-bipiperidin]-1′-yl)-N-(2-((S)-1-(3-ethoxy-4-methoxyphenyl)-2-(methylsulfonyl)ethyl)-1,3-dioxoisoindolin-4-yl)butanamide A026;
4-(5-(4-(3-(2,6-dioxopiperidin-3-yl)phenyl)-[1,4′-bipiperidin]-1′-yl)pentyl)-2-((S)-1-(3-ethoxy-4-methoxyphenyl)-2-(methylsulfonyl)ethyl)isoindoline-1,3-dione A027;
5-(1-((1′-(4-(2,6-dioxopiperidin-3-yl)phenyl)-[1,4′-bipiperidin]-4-yl)methyl)-piperidin-4-yl)-2-((S)-1-(3-ethoxy-4-methoxyphenyl)-2-(methylsulfonyl)ethyl)isoindoline-1,3-dione A028;
5-(1′-(2-(4-(4-(2,6-dioxopiperidin-3-yl)phenyl)piperidin-1-yl)ethyl)-[4,4′-bipiperidin]-1-yl)-2-((S)-1-(3-ethoxy-4-methoxyphenyl)-2-(methylsulfonyl)ethyl)isoindoline-1,3-dione A029;
5-(1′-(4-(2,6-dioxopiperidin-3-yl)phenyl)-[4,4′-bipiperidin]-1-yl)-2-((S)-1-(3-ethoxy-4-methoxyphenyl)-2-(methylsulfonyl)ethyl)isoindoline-1,3-dione A030;
4-(4-(2-(4-(4-(2,6-dioxopiperidin-3-yl)phenyl)-[1,4′-bipiperidin]-1′-yl)ethyl)-piperidin-1-yl)-2-((S)-1-(3-ethoxy-4-methoxyphenyl)-2-(methylsulfonyl)ethyl)isoindoline-1,3-dione A031;
4-(4-(1-(2-(4-(4-(2,6-dioxopiperidin-3-yl)phenyl)piperazin-1-yl)ethyl)piperidin-4-yl)piperazin-1-yl)-2-((S)-1-(3-ethoxy-4-methoxyphenyl)-2-(methylsulfonyl)ethyl)isoindoline-1,3-dione A032;
5-(4-(1-(2-(4-(4-(2,6-dioxopiperidin-3-yl)phenyl)piperazin-1-yl)ethyl)piperidin-4-yl)piperazin-1-yl)-2-((S)-1-(3-ethoxy-4-methoxyphenyl)-2-(methylsulfonyl)ethyl)isoindoline-1,3-dione A033;
5-(4-(2-(1′-(4-(2,6-dioxopiperidin-3-yl)phenyl)-[4,4′-bipiperidin]-1-yl)ethyl)-piperazin-1-yl)-2-((S)-1-(3-ethoxy-4-methoxyphenyl)-2-(methylsulfonyl)ethyl)isoindoline-1,3-dione A034;
4-(4-(2-(1′-(4-(2,6-dioxopiperidin-3-yl)phenyl)-[4,4′-bipiperidin]-1-yl)ethyl)-piperazin-1-yl)-2-((S)-1-(3-ethoxy-4-methoxyphenyl)-2-(methylsulfonyl)ethyl)isoindoline-1,3-dione A035;
4-((1-(2-(4-(4-(2,6-dioxopiperidin-3-yl)phenyl)piperidin-1-yl)ethyl)piperidin-4-yl)amino)-2-((S)-1-(3-ethoxy-4-methoxyphenyl)-2-(methylsulfonyl)ethyl)isoindoline-1,3-dione A036;
4-((1-((1-(4-(2,6-dioxopiperidin-3-yl)benzyl)piperidin-4-yl)methyl)piperidin-4-yl)amino)-2-((S)-1-(3-ethoxy-4-methoxyphenyl)-2-(methylsulfonyl)ethyl)isoindoline-1,3-dione A037;
5-(4-(4-(4-(3-(2,6-dioxopiperidin-3-yl)phenyl)but-3-yn-1-yl)piperazin-1-yl)-piperidin-1-yl)-2-((S)-1-(3-ethoxy-4-methoxyphenyl)-2-(methylsulfonyl)ethyl)isoindoline-1,3-dione A038;
5-(4-((3-(2,6-dioxopiperidin-3-yl)phenyl)ethynyl)-[1,4′-bipiperidin]-1′-yl)-2-((S)-1-(3-ethoxy-4-methoxyphenyl)-2-(methylsulfonyl)ethyl)isoindoline-1,3-dione A039;
5-(4-(4-(4-(4-(2,6-dioxopiperidin-3-yl)phenyl)but-3-yn-1-yl)piperazin-1-yl)-piperidin-1-yl)-2-((S)-1-(3-ethoxy-4-methoxyphenyl)-2-(methylsulfonyl)ethyl)isoindoline-1,3-dione A040;
5-(4-((4-(2,6-dioxopiperidin-3-yl)phenyl)ethynyl)-[1,4′-bipiperidin]-1′-yl)-2-((S)-1-(3-ethoxy-4-methoxyphenyl)-2-(methylsulfonyl)ethyl)isoindoline-1,3-dione A041;
5-(4-((4-((4-(4-(2,6-dioxopiperidin-3-yl)phenyl)piperidin-1-yl)methyl)piperidin-1-yl)methyl)piperidin-1-yl)-2-((S)-1-(3-ethoxy-4-methoxyphenyl)-2-(methylsulfonyl)ethyl)-isoindoline-1,3-dione A042;
5-((1-((1-(2-(4-(4-(2,6-dioxopiperidin-3-yl)phenyl)piperidin-1-yl)ethyl)piperidin-4-yl)methyl)piperidin-4-yl)amino)-2-((S)-1-(3-ethoxy-4-methoxyphenyl)-2-(methylsulfonyl)-ethyl)isoindoline-1,3-dione A043;
5-(4-((1′-(4-(2,6-dioxopiperidin-3-yl)phenyl)-[4,4′-bipiperidin]-1-yl)methyl)-piperidin-1-yl)-2-((S)-1-(3-ethoxy-4-methoxyphenyl)-2-(methylsulfonyl)ethyl)isoindoline-1,3-dione A044;
5-(4-(3-(4-(2,6-dioxopiperidin-3-yl)phenyl)-3-azaspiro[5.5]undecan-9-yl)-piperazin-1-yl)-2-((S)-1-(3-ethoxy-4-methoxyphenyl)-2-(methylsulfonyl)ethyl)isoindoline-1,3-dione A045;
5-(4-((1-(4-(2,6-dioxopiperidin-3-yl)phenyl)piperidin-4-yl)methyl)piperazin-1-yl)-2-((S)-1-(3-ethoxy-4-methoxyphenyl)-2-(methylsulfonyl)ethyl)isoindoline-1,3-dione A046;
4-(4-(1-(4-(4-(2,6-dioxopiperidin-3-yl)phenyl)cyclohexyl)piperidin-4-yl)-piperazin-1-yl)-2-((S)-1-(3-ethoxy-4-methoxyphenyl)-2-(methylsulfonyl)ethyl)isoindoline-1,3-dione A047;
4-(4-(2-(4-(2,6-dioxopiperidin-3-yl)phenyl)-2-azaspiro[3.3]heptan-6-yl)-piperazin-1-yl)-2-((S)-1-(3-ethoxy-4-methoxyphenyl)-2-(methylsulfonyl)ethyl)isoindoline-1,3-dione A048;
5-(4-(2-(4-(2,6-dioxopiperidin-3-yl)phenyl)-2-azaspiro[3.3]heptan-6-yl)-piperazin-1-yl)-2-((S)-1-(3-ethoxy-4-methoxyphenyl)-2-(methylsulfonyl)ethyl)isoindoline-1,3-dione A049;
4-(4-(1-(3-(2,6-dioxopiperidin-3-yl)phenethyl)piperidin-4-yl)piperazin-1-yl)-2-((S)-1-(3-ethoxy-4-methoxyphenyl)-2-(methylsulfonyl)ethyl)isoindoline-1,3-dione A050;
5-(4-(1-(3-(2,6-dioxopiperidin-3-yl)phenethyl)piperidin-4-yl)piperazin-1-yl)-2-((S)-1-(3-ethoxy-4-methoxyphenyl)-2-(methylsulfonyl)ethyl)isoindoline-1,3-dione A051;
4-(4-((1-(2-(4-(4-(2,6-dioxopiperidin-3-yl)phenyl)piperidin-1-yl)ethyl)piperidin-4-yl)methyl)piperazin-1-yl)-2-((S)-1-(3-ethoxy-4-methoxyphenyl)-2-(methylsulfonyl)ethyl)-isoindoline-1,3-dione A052;
4-((1-((1-(2-(4-(4-(2,6-dioxopiperidin-3-yl)phenyl)piperidin-1-yl)ethyl)piperidin-4-yl)methyl)piperidin-4-yl)amino)-2-((S)-1-(3-ethoxy-4-methoxyphenyl)-2-(methylsulfonyl)-ethyl)isoindoline-1,3-dione A053;
4-(4-(4-(4-(2,6-dioxopiperidin-3-yl)phenyl)cyclohexyl)piperazin-1-yl)-2-((S)-1-(3-ethoxy-4-methoxyphenyl)-2-(methylsulfonyl)ethyl)isoindoline-1,3-dione A054;
5-(4-(4-(2,6-dioxopiperidin-3-yl)phenethyl)piperazin-1-yl)-2-((S)-1-(3-ethoxy-4-methoxyphenyl)-2-(methylsulfonyl)ethyl)isoindoline-1,3-dione A055;
5-(4-(1-(4-(4-(2,6-dioxopiperidin-3-yl)phenyl)cyclohexyl)piperidin-4-yl)-piperazin-1-yl)-2-((S)-1-(3-ethoxy-4-methoxyphenyl)-2-(methylsulfonyl)ethyl)isoindoline-1,3-dione A056;
4-(4-(1-(4-(2,6-dioxopiperidin-3-yl)phenethyl)piperidin-4-yl)piperazin-1-yl)-2-((S)-1-(3-ethoxy-4-methoxyphenyl)-2-(methylsulfonyl)ethyl)isoindoline-1,3-dione A057;
5-(4-(1-(4-(2,6-dioxopiperidin-3-yl)phenethyl)piperidin-4-yl)piperazin-1-yl)-2-((S)-1-(3-ethoxy-4-methoxyphenyl)-2-(methylsulfonyl)ethyl)isoindoline-1,3-dione A058;
5-(1-(4-(4-(4-(2,6-dioxopiperidin-3-yl)phenyl)-[1,4′-bipiperidin]-1′-yl)butyl)-piperidin-4-yl)-2-((1S)-1-(3-ethoxy-4-methoxyphenyl)-2-(methylsulfinyl)ethyl)isoindoline-1,3-dione A059;
5-(1-(2-(4-(4-(2,6-dioxopiperidin-3-yl)phenyl)-[1,4′-bipiperidin]-1′-yl)ethyl)-piperidin-4-yl)-2-((S)-1-(3-ethoxy-4-methoxyphenyl)-2-(methylsulfonyl)ethyl)isoindoline-1,3-dione A060;
5-(1-(3-(4-(4-(2,6-dioxopiperidin-3-yl)phenyl)-[1,4′-bipiperidin]-1′-yl)propyl)-piperidin-4-yl)-2-((S)-1-(3-ethoxy-4-methoxyphenyl)-2-(methylsulfonyl)ethyl)isoindoline-1,3-dione A061;
4-(1-(2-(4-(4-(2,6-dioxopiperidin-3-yl)phenyl)-[1,4′-bipiperidin]-1′-yl)ethyl)-piperidin-4-yl)-2-((S)-1-(3-ethoxy-4-methoxyphenyl)-2-(methylsulfonyl)ethyl)isoindoline-1,3-dione A062;
4-(1-(3-(4-(4-(2,6-dioxopiperidin-3-yl)phenyl)-[1,4′-bipiperidin]-1′-yl)propyl)-piperidin-4-yl)-2-((S)-1-(3-ethoxy-4-methoxyphenyl)-2-(methylsulfonyl)ethyl)isoindoline-1,3-dione A063;
4-(1-(4-(4-(4-(2,6-dioxopiperidin-3-yl)phenyl)-[1,4′-bipiperidin]-1′-yl)butyl)-piperidin-4-yl)-2-((S)-1-(3-ethoxy-4-methoxyphenyl)-2-(methylsulfonyl)ethyl)isoindoline-1,3-dione A064;
5-(5-(4-(4-(2,6-dioxopiperidin-3-yl)phenyl)-[1,4′-bipiperidin]-1′-yl)pentyl)-2-((S)-1-(3-ethoxy-4-methoxyphenyl)-2-(methylsulfonyl)ethyl)isoindoline-1,3-dione A065;
5-(4-(4-(4-(2,6-dioxopiperidin-3-yl)phenyl)-[1,4′-bipiperidin]-1′-yl)butyl)-2-((S)-1-(3-ethoxy-4-methoxyphenyl)-2-(methylsulfonyl)ethyl)isoindoline-1,3-dione A066;
4-(4-(4-(4-(2,6-dioxopiperidin-3-yl)phenyl)-[1,4′-bipiperidin]-1′-yl)butyl)-2-((S)-1-(3-ethoxy-4-methoxyphenyl)-2-(methylsulfonyl)ethyl)isoindoline-1,3-dione A067;
4-(5-(4-(4-(2,6-dioxopiperidin-3-yl)phenyl)-[1,4′-bipiperidin]-1′-yl)pentyl)-2-((S)-1-(3-ethoxy-4-methoxyphenyl)-2-(methylsulfonyl)ethyl)isoindoline-1,3-dione A068;
5-(2-(4-(4-(2,6-dioxopiperidin-3-yl)phenyl)-[1,4′-bipiperidin]-1′-yl)ethyl)-2-((S)-1-(3-ethoxy-4-methoxyphenyl)-2-(methylsulfonyl)ethyl)isoindoline-1,3-dione A069;
5-(3-(4-(4-(2,6-dioxopiperidin-3-yl)phenyl)-[1,4′-bipiperidin]-1′-yl)propyl)-2-((S)-1-(3-ethoxy-4-methoxyphenyl)-2-(methylsulfonyl)ethyl)isoindoline-1,3-dione A070;
4-(2-(4-(4-(2,6-dioxopiperidin-3-yl)phenyl)-[1,4′-bipiperidin]-1′-yl)ethyl)-2-((S)-1-(3-ethoxy-4-methoxyphenyl)-2-(methylsulfonyl)ethyl)isoindoline-1,3-dione A071;
4-(3-(4-(4-(2,6-dioxopiperidin-3-yl)phenyl)-[1,4′-bipiperidin]-1′-yl)propyl)-2-((S)-1-(3-ethoxy-4-methoxyphenyl)-2-(methylsulfonyl)ethyl)isoindoline-1,3-dione A072;
4-(6-(4-(2-(4-(4-(2,6-dioxopiperidin-3-yl)phenyl)piperidin-1-yl)-2-oxoethyl)-piperidin-1-yl)hexyl)-2-((S)-1-(3-ethoxy-4-methoxyphenyl)-2-(methylsulfonyl)ethyl)isoindoline-1,3-dione A073;
4-(11-(4-(3-(2,6-dioxopiperidin-3-yl)phenyl)piperidin-1-yl)undecyl)-2-((S)-1-(3-ethoxy-4-methoxyphenyl)-2-(methylsulfonyl)ethyl)isoindoline-1,3-dione A074;
4-(11-(4-(4-(2,6-dioxopiperidin-3-yl)phenyl)piperidin-1-yl)-11-oxoundecyl)-2-((S)-1-(3-ethoxy-4-methoxyphenyl)-2-(methylsulfonyl)ethyl)isoindoline-1,3-dione A075;
4-(11-(4-(4-(2,6-dioxopiperidin-3-yl)phenyl)piperidin-1-yl)undecyl)-2-((S)-1-(3-ethoxy-4-methoxyphenyl)-2-(methylsulfonyl)ethyl)isoindoline-1,3-dione A076; or
5-(1′-(4-(4-(2,6-dioxopiperidin-3-yl)phenoxy)benzyl)-[4,4′-bipiperidin]-1-yl)-2-((S)-1-(3-ethoxy-4-methoxyphenyl)-2-(methylsulfonyl)ethyl)isoindoline-1,3-dione A077;
   or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In another embodiment, provided herein is:
5-(5-(4-(4-((2,6-dioxopiperidin-3-yl)amino)phenyl)-[1,4′-bipiperidin]-1′-yl)-pentyl)-2-((S)-1-(3-ethoxy-4-methoxyphenyl)-2-(methylsulfonyl)ethyl)isoindoline-1,3-dione A081;
5-((1-((1-(2-(4-(4-((2,6-dioxopiperidin-3-yl)amino)-2-fluoro-5-methoxyphenyl)-piperazin-1-yl)ethyl)piperidin-4-yl)methyl)piperidin-4-yl)amino)-2-((S)-1-(3-ethoxy-4-methoxy-phenyl)-2-(methylsulfonyl)ethyl)isoindoline-1,3-dione A082;
5-(4-(1-((1-(4-((2,6-dioxopiperidin-3-yl)amino)phenyl)piperidin-4-yl)methyl)-piperidin-4-yl)piperazin-1-yl)-2-((S)-1-(3-ethoxy-4-methoxyphenyl)-2-(methylsulfonyl)ethyl)-isoindoline-1,3-dione A083;
5-(4-(2-(4-(4-((2,6-dioxopiperidin-3-yl)amino)phenethyl)piperazin-1-yl)ethyl)-piperidin-1-yl)-2-((S)-1-(3-ethoxy-4-methoxyphenyl)-2-(methylsulfonyl)ethyl)isoindoline-1,3-dione A084;
4-((1-((1-(2-(4-(4-((2,6-dioxopiperidin-3-yl)amino)-2-fluorophenyl)piperidin-1-yl)ethyl)piperidin-4-yl)methyl)piperidin-4-yl)amino)-2-((S)-1-(3-ethoxy-4-methoxyphenyl)-2-(methylsulfonyl)ethyl)isoindoline-1,3-dione A085;
5-(4-((4-((4-(4-((2,6-dioxopiperidin-3-yl)amino)phenyl)piperidin-1-yl)methyl)-piperidin-1-yl)methyl)piperidin-1-yl)-2-((S)-1-(3-ethoxy-4-methoxyphenyl)-2-(methylsulfonyl)-ethyl)isoindoline-1,3-dione A086;
5-(4-((1-(2-((4-((2,6-dioxopiperidin-3-yl)amino)phenyl)amino)ethyl)piperidin-4-yl)methyl)piperazin-1-yl)-2-((S)-1-(3-ethoxy-4-methoxyphenyl)-2-(methylsulfonyl)ethyl)-isoindoline-1,3-dione A087; or
5-(1′-(4-(4-((2,6-dioxopiperidin-3-yl)amino)phenoxy)benzyl)-[4,4′-bipiperidin]-1-yl)-2-((S)-1-(3-ethoxy-4-methoxyphenyl)-2-(methylsulfonyl)ethyl)isoindoline-1,3-dione A088;
   or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In yet another embodiment, provided herein is:
5-(1′-(4-((3-(2,6-dioxopiperidin-3-yl)-1-methyl-1H-indazol-6-yl)oxy)benzyl)-[4,4′-bipiperidin]-1-yl)-2-((S)-1-(3-ethoxy-4-methoxyphenyl)-2-(methylsulfonyl)ethyl)-isoindoline-1,3-dione B001,
5-(4-((1-(4-((3-(2,6-dioxopiperidin-3-yl)-1-methyl-1H-indazol-6-yl)oxy)benzyl)-piperidin-4-yl)methyl)piperazin-1-yl)-2-((S)-1-(3-ethoxy-4-methoxyphenyl)-2-(methylsulfonyl)-ethyl)isoindoline-1,3-dione B002;
4-(1′-(4-((3-(2,6-dioxopiperidin-3-yl)-1-methyl-1H-indazol-6-yl)oxy)benzyl)-[4,4′-bipiperidin]-1-yl)-2-((S)-1-(3-ethoxy-4-methoxyphenyl)-2-(methylsulfonyl)ethyl)-isoindoline-1,3-dione B003;
5-(1-((1-(4-((3-(2,6-Dioxopiperidin-3-yl)-1-methyl-1H-indazol-6-yl)oxy)benzyl)-piperidin-4-yl)methyl)piperidin-4-yl)-2-((S)-1-(3-ethoxy-4-methoxyphenyl)-2-(methylsulfonyl)-ethyl)isoindoline-1,3-dione B004;
3-(6-(4-((1′-(2-((S)-1-(3-ethoxy-4-methoxyphenyl)-2-(methylsulfonyl)ethyl)-1-oxoisoindolin-5-yl)-[4,4′-bipiperidin]-1-yl)methyl)phenoxy)-1-methyl-1H-indazol-3-yl)-piperidine-2,6-dione B005;
3-(6-(4-((1′-(2-((S)-1-(3-ethoxy-4-methoxyphenyl)-2-(methylsulfonyl)ethyl)-3-oxoisoindolin-5-yl)-[4,4′-bipiperidin]-1-yl)methyl)phenoxy)-1-methyl-1H-indazol-3-yl)-piperidine-2,6-dione B006; or
4-(4-((1-(4-((3-(2,6-dioxopiperidin-3-yl)-1-methyl-1H-indazol-6-yl)oxy)benzyl)-piperidin-4-yl)methyl)piperazin-1-yl)-2-((S)-1-(3-ethoxy-4-methoxyphenyl)-2-(methylsulfonyl)-ethyl)isoindoline-1,3-dione B007;
   or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In yet another embodiment, provided herein is:
5-((4′R)-1′-(4-((3-(2,6-dioxopiperidin-3-yl)-1-methyl-1H-indazol-6-yl)oxy)-benzyl)-3′,3′-difluoro-[4,4′-bipiperidin]-1-yl)-2-((S)-1-(3-ethoxy-4-methoxyphenyl)-2-(methyl-sulfonyl)ethyl)isoindoline-1,3-dione B008;
4-((4′R)-1′-(4-((3-(2,6-dioxopiperidin-3-yl)-1-methyl-1H-indazol-6-yl)oxy)-benzyl)-3′,3′-difluoro-[4,4′-bipiperidin]-1-yl)-2-((S)-1-(3-ethoxy-4-methoxyphenyl)-2-(methyl-sulfonyl)ethyl)isoindoline-1,3-dione B009;
4-(4-((4-(4-((3-(2,6-dioxopiperidin-3-yl)-1-methyl-1H-indazol-6-yl)oxy)phenyl)-piperidin-1-yl)methyl)-4-fluoropiperidin-1-yl)-2-((S)-1-(3-ethoxy-4-methoxyphenyl)-2-(methyl-sulfonyl)ethyl)isoindoline-1,3-dione B010;
4-(1′-(2-(4-((3-(2,6-dioxopiperidin-3-yl)-1-methyl-1H-indazol-6-yl)oxy)phenyl)-2,2-difluoroethyl)-[4,4′-bipiperidin]-1-yl)-2-((S)-1-(3-ethoxy-4-methoxyphenyl)-2-(methyl-sulfonyl)ethyl)isoindoline-1,3-dione B011;
4-(4-(2-(4-(4-((3-(2,6-dioxopiperidin-3-yl)-1-methyl-1H-indazol-6-yl)oxy)-phenyl)piperazin-1-yl)ethyl)piperidin-1-yl)-2-((S)-1-(3-ethoxy-4-methoxyphenyl)-2-(methyl-sulfonyl)ethyl)isoindoline-1,3-dione B012.
5-(1′-(4-((3-((S)-2,6-dioxopiperidin-3-yl)-1-methyl-1H-indazol-6-yl)oxy)benzyl)-[4,4′-bipiperidin]-1-yl)-2-((S)-1-(3-ethoxy-4-methoxyphenyl)-2-(methylsulfonyl)ethyl)-isoindoline-1,3-dione B021;
5-(1′-(4-((3-((R)-2,6-dioxopiperidin-3-yl)-1-methyl-1H-indazol-6-yl)oxy)benzyl)-[4,4′-bipiperidin]-1-yl)-2-((S)-1-(3-ethoxy-4-methoxyphenyl)-2-(methylsulfonyl)ethyl)-isoindoline-1,3-dione B022;
5-(4-((S)-3,3-difluoro-1-(4-((1-methyl-3-((S)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)isoindoline-1,3-dione B023;
5-(4-((S)-3,3-difluoro-1-(4-((1-methyl-3-((R)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)isoindoline-1,3-dione B024;
5-(4-((S)-3,3-difluoro-1-(4-((5-fluoro-1-methyl-3-((S)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)isoindoline-1,3-dione B025;
5-(4-((S)-3,3-difluoro-1-(4-((5-fluoro-1-methyl-3-((R)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)isoindoline-1,3-dione B026;
5-(4-((R)-3,3-difluoro-1-(4-((1-methyl-3-((S)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)isoindoline-1,3-dione B027;
5-(4-((R)-3,3-difluoro-1-(4-((1-methyl-3-((R)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)isoindoline-1,3-dione B028;
5-(4-((R)-3,3-difluoro-1-(4-((5-fluoro-1-methyl-3-((S)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)isoindoline-1,3-dione B029;
5-(4-((R)-3,3-difluoro-1-(4-((5-fluoro-1-methyl-3-((R)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)isoindoline-1,3-dione B030;
4-(4-((S)-3,3-difluoro-1-(4-((1-methyl-3-((S)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)isoindoline-1,3-dione B031;
4-(4-((S)-3,3-difluoro-1-(4-((1-methyl-3-((R)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)isoindoline-1,3-dione B032;
4-(4-((S)-3,3-difluoro-1-(4-((5-fluoro-1-methyl-3-((S)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)isoindoline-1,3-dione B033;
4-(4-((S)-3,3-difluoro-1-(4-((5-fluoro-1-methyl-3-((S)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)isoindoline-1,3-dione B034;
4-(4-((R)-3,3-difluoro-1-(4-((1-methyl-3-((S)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)isoindoline-1,3-dione B035;
4-(4-((R)-3,3-difluoro-1-(4-((1-methyl-3-((R)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)isoindoline-1,3-dione B036;
4-(4-((R)-3,3-difluoro-1-(4-((5-fluoro-1-methyl-3-((S)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)isoindoline-1,3-dione B037;
4-(4-((R)-3,3-difluoro-1-(4-((5-fluoro-1-methyl-3-((S)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)isoindoline-1,3-dione B038;
4-(1′-(2-(4-((3-((R)-2,6-dioxopiperidin-3-yl)-1-methyl-1H-indazol-6-yl)oxy)-phenyl)-2,2-difluoroethyl)-[4,4′-bipiperidin]-1-yl)-2-((S)-1-(3-ethoxy-4-methoxyphenyl)-2-(methylsulfonyl)ethyl)isoindoline-1,3-dione B039;
4-(1′-(2-(4-((3-((S)-2,6-dioxopiperidin-3-yl)-1-methyl-1H-indazol-6-yl)oxy)-phenyl)-2,2-difluoroethyl)-[4,4′-bipiperidin]-1-yl)-2-((S)-1-(3-ethoxy-4-methoxyphenyl)-2-(methylsulfonyl)ethyl)isoindoline-1,3-dione B040;
4-(4-(1-(2,2-difluoro-2-(4-((1-methyl-3-((S)-3-methyl-2,6-dioxopiperidin-3-yl)-1H-indazol-6-yl)oxy)phenyl)ethyl)piperidin-4-yl)piperazin-1-yl)-2-((S)-1-(3-ethoxy-4-methoxyphenyl)-2-(methylsulfonyl)ethyl)isoindoline-1,3-dione B041;
4-(4-(1-(2,2-difluoro-2-(4-((1-methyl-3-((R)-3-methyl-2,6-dioxopiperidin-3-yl)-1H-indazol-6-yl)oxy)phenyl)ethyl)piperidin-4-yl)piperazin-1-yl)-2-((S)-1-(3-ethoxy-4-methoxyphenyl)-2-(methylsulfonyl)ethyl)isoindoline-1,3-dione B042;
4-(4-(1-(2,2-difluoro-2-(4-((5-fluoro-1-methyl-3-((S)-3-methyl-2,6-dioxo-piperidin-3-yl)-1H-indazol-6-yl)oxy)phenyl)ethyl)piperidin-4-yl)piperazin-1-yl)-2-((S)-1-(3-ethoxy-4-methoxyphenyl)-2-(methylsulfonyl)ethyl)isoindoline-1,3-dione B043;
4-(4-(1-(2,2-Difluoro-2-(4-((5-fluoro-1-methyl-3-((R)-3-methyl-2,6-dioxo-piperidin-3-yl)-1H-indazol-6-yl)oxy)phenyl)ethyl)piperidin-4-yl)piperazin-1-yl)-2-((S)-1-(3-ethoxy-4-methoxyphenyl)-2-(methylsulfonyl)ethyl)isoindoline-1,3-dione B044;
4-(4-(2-(4-(4-((3-((S)-2,6-dioxopiperidin-3-yl)-1-methyl-1H-indazol-6-yl)oxy)-phenyl)piperazin-1-yl)ethyl)piperidin-1-yl)-2-((S)-1-(3-ethoxy-4-methoxyphenyl)-2-(methyl-sulfonyl)ethyl)isoindoline-1,3-dione B045;
4-(4-(2-(4-(4-((3-((R)-2,6-dioxopiperidin-3-yl)-1-methyl-1H-indazol-6-yl)oxy)-phenyl)piperazin-1-yl)ethyl)piperidin-1-yl)-2-((S)-1-(3-ethoxy-4-methoxyphenyl)-2-(methyl-sulfonyl)ethyl)isoindoline-1,3-dione B046;
2-((S)-1-(3-ethoxy-4-methoxyphenyl)-2-(methylsulfonyl)ethyl)-4-(4-(2-(1-(4-((1-methyl-3-((R)-3-methyl-2,6-dioxopiperidin-3-yl)-1H-indazol-6-yl)oxy)phenyl)piperidin-4-yl)-ethyl)piperazin-1-yl)isoindoline-1,3-dione B047;
2-((S)-1-(3-ethoxy-4-methoxyphenyl)-2-(methylsulfonyl)ethyl)-4-(4-(2-(1-(4-((5-fluoro-1-methyl-3-((R)-3-methyl-2,6-dioxopiperidin-3-yl)-1H-indazol-6-yl)oxy)phenyl)-piperidin-4-yl)ethyl)piperazin-1-yl)isoindoline-1,3-dione B048; or
4-((4'S)-1′-(4-((3-(2,6-dioxopiperidin-3-yl)-1-methyl-1H-indazol-6-yl)oxy)phen-ethyl)-3′,3′-difluoro-[4,4′-bipiperidin]-1-yl)-2-((S)-1-(3-ethoxy-4-methoxyphenyl)-2-(methyl-sulfonyl)ethyl)isoindoline-1,3-dione B049;
   or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In yet another embodiment, provided herein is:
6-(4-((S)-3,3-difluoro-1-(4-((1-methyl-3-((R)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B051;
6-(4-((S)-3,3-difluoro-1-(4-((5-fluoro-1-methyl-3-((R)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B052;
6-(4-((R)-3,3-difluoro-1-(4-((1-methyl-3-((R)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B053;
6-(4-((R)-3,3-difluoro-1-(4-((5-fluoro-1-methyl-3-((R)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B054;
4-(4-((S)-3,3-difluoro-1-(4-((1-methyl-3-((R)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B055;
4-(4-((S)-3,3-difluoro-1-(4-((5-fluoro-1-methyl-3-((R)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B056;
4-(4-((R)-3,3-difluoro-1-(4-((1-methyl-3-((R)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B057;
4-(4-((R)-3,3-difluoro-1-(4-((5-fluoro-1-methyl-3-((R)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B058;
4-(4-(1-(2,2-difluoro-2-(4-((1-methyl-3-((S)-3-methyl-2,6-dioxopiperidin-3-yl)-1H-indazol-6-yl)oxy)phenyl)ethyl)piperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B059;
4-(4-(1-(2,2-difluoro-2-(4-((1-methyl-3-((R)-3-methyl-2,6-dioxopiperidin-3-yl)-1H-indazol-6-yl)oxy)phenyl)ethyl)piperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B060; or
4-(4-(1-(2,2-difluoro-2-(4-((5-fluoro-1-methyl-3-((R)-3-methyl-2,6-dioxo-piperidin-3-yl)-1H-indazol-6-yl)oxy)phenyl)ethyl)piperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B061;
   or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In yet another embodiment, provided herein is:
5-((R)-1′-(4-((3-(2,4-dioxotetrahydropyrimidin-1(2H)-yl)-1-methyl-1H-indazol-6-yl)oxy)benzyl)-3′,3′-difluoro-[4,4′-bipiperidin]-1-yl)-2-((S)-1-(3-ethoxy-4-methoxyphenyl)-2-(methylsulfonyl)ethyl)isoindoline-1,3-dione B151;
4-((R)-1′-(4-((3-(2,4-dioxotetrahydropyrimidin-1(2H)-yl)-5-fluoro-1-methyl-1H-indazol-6-yl)oxy)benzyl)-3′,3′-difluoro-[4,4′-bipiperidin]-1-yl)-2-((S)-1-(3-ethoxy-4-methoxy-phenyl)-2-(methylsulfonyl)ethyl)isoindoline-1,3-dione B152;
4-(4-((R)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)isoindoline-1,3-dione B153;
5-((R)-1′-(4-((3-(2,4-dioxotetrahydropyrimidin-1(2H)-yl)-5-fluoro-1-methyl-1H-indazol-6-yl)oxy)benzyl)-3′,3′-difluoro-[4,4′-bipiperidin]-1-yl)-2-((S)-1-(3-ethoxy-4-methoxy-phenyl)-2-(methylsulfonyl)ethyl)isoindoline-1,3-dione B154;
5-(4-((S)-1-(4-((3-(2,4-dioxotetrahydropyrimidin-1(2H)-yl)-1-methyl-1H-indazol-6-yl)oxy)benzyl)-3,3-difluoropiperidin-4-yl)piperazin-1-yl)-2-((S)-1-(3-ethoxy-4-methoxy-phenyl)-2-(methylsulfonyl)ethyl)isoindoline-1,3-dione B155;
5-(4-((R)-1-(4-((3-(2,4-dioxotetrahydropyrimidin-1(2H)-yl)-1-methyl-1H-indazol-6-yl)oxy)benzyl)-3,3-difluoropiperidin-4-yl)piperazin-1-yl)-2-((S)-1-(3-ethoxy-4-methoxy-phenyl)-2-(methylsulfonyl)ethyl)isoindoline-1,3-dione B156;
5-(4-((S)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)isoindoline-1,3-dione B157;
5-(4-((R)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)isoindoline-1,3-dione B158;
4-((R)-1′-(4-((3-(2,4-dioxotetrahydropyrimidin-1(2H)-yl)-1-methyl-1H-indazol-6-yl)oxy)benzyl)-3′,3′-difluoro-[4,4′-bipiperidin]-1-yl)-2-((S)-1-(3-ethoxy-4-methoxyphenyl)-2-(methylsulfonyl)ethyl)isoindoline-1,3-dione B159;
4-(4-((S)-1-(4-((3-(2,4-dioxotetrahydropyrimidin-1(2H)-yl)-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)isoindoline-1,3-dione B160;
4-(4-((S)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)isoindoline-1,3-dione B161;
4-(4-((R)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)isoindoline-1,3-dione B162;
(S)-4-(4-((4-(4-((3-(2,4-dioxotetrahydropyrimidin-1(2H)-yl)-1-methyl-1H-indazol-6-yl)oxy)phenyl)piperidin-1-yl)methyl)-4-fluoropiperidin-1-yl)-2-(1-(3-ethoxy-4-methoxyphenyl)-2-(methylsulfonyl)ethyl)isoindoline-1,3-dione B163;
(S)-4-(4-((4-(4-((3-(2,4-dioxotetrahydropyrimidin-1(2H)-yl)-5-fluoro-1-methyl-1H-indazol-6-yl)oxy)phenyl)piperidin-1-yl)methyl)-4-fluoropiperidin-1-yl)-2-(1-(3-ethoxy-4-methoxyphenyl)-2-(methylsulfonyl)ethyl)isoindoline-1,3-dione B164;
(S)-5-(4-((4-(4-((3-(2,4-dioxotetrahydropyrimidin-1(2H)-yl)-1-methyl-1H-indazol-6-yl)oxy)phenyl)piperidin-1-yl)methyl)-4-fluoropiperidin-1-yl)-2-(1-(3-ethoxy-4-methoxyphenyl)-2-(methylsulfonyl)ethyl)isoindoline-1,3-dione B165;
(S)-5-(4-((4-(4-((3-(2,4-dioxotetrahydropyrimidin-1(2H)-yl)-5-fluoro-1-methyl-1H-indazol-6-yl)oxy)phenyl)piperidin-1-yl)methyl)-4-fluoropiperidin-1-yl)-2-(1-(3-ethoxy-4-methoxyphenyl)-2-(methylsulfonyl)ethyl)isoindoline-1,3-dione B166;
(S)-4-(1′-(2-(4-((3-(2,4-dioxotetrahydropyrimidin-1(2H)-yl)-1-methyl-1H-indazol-6-yl)oxy)phenyl)-2,2-difluoroethyl)-[4,4′-bipiperidin]-1-yl)-2-(1-(3-ethoxy-4-methoxy-phenyl)-2-(methylsulfonyl)ethyl)isoindoline-1,3-dione B167;
(S)-4-(1′-(2-(4-((3-(2,4-dioxotetrahydropyrimidin-1(2H)-yl)-5-fluoro-1-methyl-1H-indazol-6-yl)oxy)phenyl)-2,2-difluoroethyl)-[4,4′-bipiperidin]-1-yl)-2-(1-(3-ethoxy-4-methoxyphenyl)-2-(methylsulfonyl)ethyl)isoindoline-1,3-dione B168;
(S)-4-(4-(1-(2-(4-((3-(2,4-dioxotetrahydropyrimidin-1(2H)-yl)-1-methyl-1H-indazol-6-yl)oxy)phenyl)-2,2-difluoroethyl)piperidin-4-yl)piperazin-1-yl)-2-(1-(3-ethoxy-4-methoxyphenyl)-2-(methylsulfonyl)ethyl)isoindoline-1,3-dione B169;
(S)-4-(4-(1-(2-(4-((3-(2,4-dioxotetrahydropyrimidin-1(2H)-yl)-5-fluoro-1-methyl-1H-indazol-6-yl)oxy)phenyl)-2,2-difluoroethyl)piperidin-4-yl)piperazin-1-yl)-2-(1-(3-ethoxy-4-methoxyphenyl)-2-(methylsulfonyl)ethyl)isoindoline-1,3-dione B170;
(S)-4-(4-(2-(4-(4-((3-(2,4-dioxotetrahydropyrimidin-1(2H)-yl)-1-methyl-1H-indazol-6-yl)oxy)phenyl)piperazin-1-yl)ethyl)piperidin-1-yl)-2-(1-(3-ethoxy-4-methoxyphenyl)-2-(methylsulfonyl)ethyl)isoindoline-1,3-dione B171;
(S)-4-(4-(2-(4-(4-((3-(2,4-dioxotetrahydropyrimidin-1(2H)-yl)-5-fluoro-1-methyl-1H-indazol-6-yl)oxy)phenyl)piperazin-1-yl)ethyl)piperidin-1-yl)-2-(1-(3-ethoxy-4-methoxyphenyl)-2-(methylsulfonyl)ethyl)isoindoline-1,3-dione B172;
(S)-4-(4-(2-(1-(4-((3-(2,4-dioxotetrahydropyrimidin-1(2H)-yl)-1-methyl-1H-indazol-6-yl)oxy)phenyl)piperidin-4-yl)ethyl)piperazin-1-yl)-2-(1-(3-ethoxy-4-methoxyphenyl)-2-(methylsulfonyl)ethyl)isoindoline-1,3-dione B173;
(S)-4-(4-(2-(1-(4-((3-(2,4-dioxotetrahydropyrimidin-1(2H)-yl)-5-fluoro-1-methyl-1H-indazol-6-yl)oxy)phenyl)piperidin-4-yl)ethyl)piperazin-1-yl)-2-(1-(3-ethoxy-4-methoxyphenyl)-2-(methylsulfonyl)ethyl)isoindoline-1,3-dione B174;
4-((S)-1′-(4-((3-(2,4-dioxotetrahydropyrimidin-1(2H)-yl)-1-methyl-1H-indazol-6-yl)oxy)phenethyl)-3′,3′-difluoro-[4,4′-bipiperidin]-1-yl)-2-((S)-1-(3-ethoxy-4-methoxyphenyl)-2-(methylsulfonyl)ethyl)isoindoline-1,3-dione B175;
4-((S)-1′-(4-((3-(2,4-dioxotetrahydropyrimidin-1(2H)-yl)-5-fluoro-1-methyl-1H-indazol-6-yl)oxy)phenethyl)-3′,3′-difluoro-[4,4′-bipiperidin]-1-yl)-2-((S)-1-(3-ethoxy-4-methoxy-phenyl)-2-(methylsulfonyl)ethyl)isoindoline-1,3-dione B176;
4-(4-((R)-1-(4-((3-(2,4-dioxotetrahydropyrimidin-1(2H)-yl)-1-methyl-1H-indazol-6-yl)oxy)phenethyl)-3,3-difluoropiperidin-4-yl)piperazin-1-yl)-2-((S)-1-(3-ethoxy-4-methoxyphenyl)-2-(methylsulfonyl)ethyl)isoindoline-1,3-dione B177;
4-(4-((R)-1-(4-((3-(2,4-dioxotetrahydropyrimidin-1(2H)-yl)-5-fluoro-1-methyl-1H-indazol-6-yl)oxy)phenethyl)-3,3-difluoropiperidin-4-yl)piperazin-1-yl)-2-((S)-1-(3-ethoxy-4-methoxyphenyl)-2-(methylsulfonyl)ethyl)isoindoline-1,3-dione B178;
4-(4-((S)-1-(4-((3-(2,4-dioxotetrahydropyrimidin-1(2H)-yl)-1-methyl-1H-indazol-6-yl)oxy)phenethyl)-3,3-difluoropiperidin-4-yl)piperazin-1-yl)-2-((S)-1-(3-ethoxy-4-methoxyphenyl)-2-(methylsulfonyl)ethyl)isoindoline-1,3-dione B179; or
4-(4-((S)-1-(4-((3-(2,4-dioxotetrahydropyrimidin-1(2H)-yl)-5-fluoro-1-methyl-1H-indazol-6-yl)oxy)phenethyl)-3,3-difluoropiperidin-4-yl)piperazin-1-yl)-2-((S)-1-(3-ethoxy-4-methoxyphenyl)-2-(methylsulfonyl)ethyl)isoindoline-1,3-dione B180;
   or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof.

In yet another embodiment, provided herein is:
6-((R)-1′-(4-((3-(2,4-dioxotetrahydropyrimidin-1(2H)-yl)-1-methyl-1H-indazol-6-yl)oxy)benzyl)-3′,3′-difluoro-[4,4′-bipiperidin]-1-yl)-2-((S)-1-(3-ethoxy-4-methoxyphenyl)-2-(methylsulfonyl)ethyl)-1H-pyrrolo[3,4-c]pyridine-1,3(2H)-dione B201;
6-((R)-1′-(4-((3-(2,4-dioxotetrahydropyrimidin-1(2H)-yl)-5-fluoro-1-methyl-1H-indazol-6-yl)oxy)benzyl)-3′,3′-difluoro-[4,4′-bipiperidin]-1-yl)-2-((S)-1-(3-ethoxy-4-methoxy-phenyl)-2-(methylsulfonyl)ethyl)-1H-pyrrolo[3,4-c]pyridine-1,3(2H)-dione B202;
6-(4-((S)-1-(4-((3-(2,4-dioxotetrahydropyrimidin-1(2H)-yl)-1-methyl-1H-indazol-6-yl)oxy)benzyl)-3,3-difluoropiperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B203;
6-(4-((S)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B204;
6-(4-((R)-1-(4-((3-(2,4-dioxotetrahydropyrimidin-1(2H)-yl)-1-methyl-1H-indazol-6-yl)oxy)benzyl)-3,3-difluoropiperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B205;
6-(4-((R)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B206;
4-((R)-1′-(4-((3-(2,4-dioxotetrahydropyrimidin-1(2H)-yl)-1-methyl-1H-indazol-6-yl)oxy)benzyl)-3′,3′-difluoro-[4,4′-bipiperidin]-1-yl)-2-((S)-1-(3-ethoxy-4-methoxyphenyl)-2-(methylsulfonyl)ethyl)-1H-pyrrolo[3,4-c]pyridine-1,3(2H)-dione B207;
4-((R)-1′-(4-((3-(2,4-dioxotetrahydropyrimidin-1(2H)-yl)-5-fluoro-1-methyl-1H-indazol-6-yl)oxy)benzyl)-3′,3′-difluoro-[4,4′-bipiperidin]-1-yl)-2-((S)-1-(3-ethoxy-4-methoxy-phenyl)-2-(methylsulfonyl)ethyl)-1H-pyrrolo[3,4-c]pyridine-1,3(2H)-dione B208;
4-(4-((S)-1-(4-((3-(2,4-dioxotetrahydropyrimidin-1(2H)-yl)-1-methyl-1H-indazol-6-yl)oxy)benzyl)-3,3-difluoropiperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B209;
4-(4-((S)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B210;
4-(4-((R)-1-(4-((3-(2,4-dioxotetrahydropyrimidin-1(2H)-yl)-1-methyl-1H-indazol-6-yl)oxy)benzyl)-3,3-difluoropiperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B211;
4-(4-((R)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B212;
(S)-4-(4-(1-(2-(4-((3-(2,4-dioxotetrahydropyrimidin-1(2H)-yl)-5-fluoro-1-methyl-1H-indazol-6-yl)oxy)phenyl)-2,2-difluoroethyl)piperidin-4-yl)piperazin-1-yl)-2-(1-(3-ethoxy-4-methoxyphenyl)-2-(methylsulfonyl)ethyl)-1H-pyrrolo[3,4-c]pyridine-1,3(2H)-dione B213;
(S)-4-(4-(2-(4-(4-((3-(2,4-dioxotetrahydropyrimidin-1(2H)-yl)-1-methyl-1H-indazol-6-yl)oxy)phenyl)piperazin-1-yl)ethyl)piperidin-1-yl)-2-(1-(3-ethoxy-4-methoxyphenyl)-2-(methylsulfonyl)ethyl)-1H-pyrrolo[3,4-c]pyridine-1,3(2H)-dione B214;
(S)-4-(4-(2-(4-(4-((3-(2,4-dioxotetrahydropyrimidin-1(2H)-yl)-5-fluoro-1-methyl-1H-indazol-6-yl)oxy)phenyl)piperazin-1-yl)ethyl)piperidin-1-yl)-2-(1-(3-ethoxy-4-methoxy-phenyl)-2-(methylsulfonyl)ethyl)-1H-pyrrolo[3,4-c]pyridine-1,3(2H)-dione B215;
(S)-4-(4-(2-(1-(4-((3-(2,4-dioxotetrahydropyrimidin-1(2H)-yl)-1-methyl-1H-indazol-6-yl)oxy)phenyl)piperidin-4-yl)ethyl)piperazin-1-yl)-2-(1-(3-ethoxy-4-methoxyphenyl)-2-(methylsulfonyl)ethyl)-1H-pyrrolo[3,4-c]pyridine-1,3(2H)-dione B216;
(S)-4-(4-(2-(1-(4-((3-(2,4-dioxotetrahydropyrimidin-1(2H)-yl)-5-fluoro-1-methyl-1H-indazol-6-yl)oxy)phenyl)piperidin-4-yl)ethyl)piperazin-1-yl)-2-(1-(3-ethoxy-4-methoxy-phenyl)-2-(methylsulfonyl)ethyl)-1H-pyrrolo[3,4-c]pyridine-1,3(2H)-dione B217;
4-((S)-1′-(4-((3-(2,4-dioxotetrahydropyrimidin-1(2H)-yl)-1-methyl-1H-indazol-6-yl)oxy)phenethyl)-3′,3′-difluoro-[4,4′-bipiperidin]-1-yl)-2-((S)-1-(3-ethoxy-4-methoxyphenyl)-2-(methylsulfonyl)ethyl)-1H-pyrrolo[3,4-c]pyridine-1,3(2H)-dione B218;
4-((S)-1′-(4-((3-(2,4-dioxotetrahydropyrimidin-1(2H)-yl)-5-fluoro-1-methyl-1H-indazol-6-yl)oxy)phenethyl)-3′,3′-difluoro-[4,4′-bipiperidin]-1-yl)-2-((S)-1-(3-ethoxy-4-methoxy-phenyl)-2-(methylsulfonyl)ethyl)-1H-pyrrolo[3,4-c]pyridine-1,3(2H)-dione B219;
4-(4-((R)-1-(4-((3-(2,4-dioxotetrahydropyrimidin-1(2H)-yl)-1-methyl-1H-indazol-6-yl)oxy)phenethyl)-3,3-difluoropiperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B220;
4-(4-((R)-1-(4-((3-(2,4-dioxotetrahydropyrimidin-1(2H)-yl)-5-fluoro-1-methyl-1H-indazol-6-yl)oxy)phenethyl)-3,3-difluoropiperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B221;
4-(4-((S)-1-(4-((3-(2,4-dioxotetrahydropyrimidin-1(2H)-yl)-1-methyl-1H-indazol-6-yl)oxy)phenethyl)-3,3-difluoropiperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B222; or
4-(4-((S)-1-(4-((3-(2,4-dioxotetrahydropyrimidin-1(2H)-yl)-5-fluoro-1-methyl-1H-indazol-6-yl)oxy)phenethyl)-3,3-difluoropiperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B223.
   or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof.

In yet another embodiment, provided herein is:
(3R)-3-(5-(4-((4-((4-(4-(2,6-dioxopiperidin-3-yl)phenyl)piperidin-1-yl)methyl)-piperidin-1-yl)methyl)piperidin-1-yl)-1,3-dioxoisoindolin-2-yl)-3-(3-ethoxy-4-methoxyphenyl)-propanenitrile C001;
(3R)-3-(5-(4-((4-((4-(4-((2,6-dioxopiperidin-3-yl)amino)phenyl)piperidin-1-yl)-methyl)piperidin-1-yl)methyl)piperidin-1-yl)-1,3-dioxoisoindolin-2-yl)-3-(3-ethoxy-4-methoxy-phenyl)propanenitrile C002;
(3R)-3-(6-(1′-(4-((3-(2,6-dioxopiperidin-3-yl)-1-methyl-1H-indazol-6-yl)oxy)-benzyl)-[4,4′-bipiperidin]-1-yl)-1-oxoisoindolin-2-yl)-3-(3-ethoxy-4-methoxyphenyl)propanenitrile C011;
(3R)-3-(5-(1′-(4-((3-(2,6-dioxopiperidin-3-yl)-1-methyl-1H-indazol-6-yl)oxy)-benzyl)-[4,4′-bipiperidin]-1-yl)-1,3-dioxoisoindolin-2-yl)-3-(3-ethoxy-4-methoxyphenyl)-propanenitrile C012;
(3R)-3-(6-(1′-(4-((3-(2,6-dioxopiperidin-3-yl)-1-methyl-1H-indazol-6-yl)oxy)-benzyl)-[4,4′-bipiperidin]-1-yl)-1-oxoisoindolin-2-yl)-3-(3-ethoxy-4-methoxyphenyl)propanenitrile C013;
(3R)-3-(5-(1′-(4-((3-(2,6-dioxopiperidin-3-yl)-1-methyl-1H-indazol-6-yl)oxy)-benzyl)-[4,4′-bipiperidin]-1-yl)-1-oxoisoindolin-2-yl)-3-(3-ethoxy-4-methoxyphenyl)propanenitrile C014;
(3R)-3-(4-(4-((1-(4-((3-(2,6-dioxopiperidin-3-yl)-1-methyl-1H-indazol-6-yl)oxy)-benzyl)piperidin-4-yl)methyl)piperazin-1-yl)-1,3-dioxoisoindolin-2-yl)-3-(3-ethoxy-4-methoxy-phenyl)propanenitrile C015;
(3R)-3-(5-(4-((1-(4-((3-(2,6-dioxopiperidin-3-yl)-1-methyl-1H-indazol-6-yl)oxy)-benzyl)piperidin-4-yl)methyl)piperazin-1-yl)-1,3-dioxoisoindolin-2-yl)-3-(3-ethoxy-4-methoxy-phenyl)propanenitrile C016;
(3R)-3-(5-(1-((1-(4-((3-(2,6-dioxopiperidin-3-yl)-1-methyl-1H-indazol-6-yl)oxy)-benzyl)piperidin-4-yl)methyl)piperidin-4-yl)-1,3-dioxoisoindolin-2-yl)-3-(3-ethoxy-4-methoxy-phenyl)propanenitrile C017;
(3R)-3-(4-(1′-(4-((3-(2,6-dioxopiperidin-3-yl)-1-methyl-1H-indazol-6-yl)oxy)-benzyl)-[4,4′-bipiperidin]-1-yl)-1,3-dioxoisoindolin-2-yl)-3-(3-ethoxy-4-methoxyphenyl)-propanenitrile C018;
(3R)-3-(5-(4-((4-((4-(3-(2,6-dioxopiperidin-3-yl)-1-methyl-1H-indazol-6-yl)-piperidin-1-yl)methyl)piperidin-1-yl)methyl)piperidin-1-yl)-1,3-dioxoisoindolin-2-yl)-3-(3-ethoxy-4-methoxyphenyl)propanenitrile C019;
(3R)-3-(4-((1-((1-(2-(4-(3-(2,6-dioxopiperidin-3-yl)-1-methyl-1H-indazol-6-yl)-piperazin-1-yl)ethyl)piperidin-4-yl)methyl)piperidin-4-yl)amino)-1,3-dioxoisoindolin-2-yl)-3-(3-ethoxy-4-methoxyphenyl)propanenitrile C020;
(3R)-3-(4-(4-((1-(2-(4-(3-(2,6-dioxopiperidin-3-yl)-1-methyl-1H-indazol-6-yl)-piperidin-1-yl)acetyl)piperidin-4-yl)methyl)piperazin-1-yl)-1,3-dioxoisoindolin-2-yl)-3-(3-ethoxy-4-methoxyphenyl)propanenitrile C021;
(3R)-3-(5-(4-((4-((3-(2,6-dioxopiperidin-3-yl)-1-methyl-1H-indazol-6-yl)oxy)-piperidin-1-yl)methyl)-[1,4′-bipiperidin]-1′-yl)-1,3-dioxoisoindolin-2-yl)-3-(3-ethoxy-4-methoxyphenyl)propanenitrile C022; or
2-(4-(11-(2-((R)-2-cyano-1-(3-ethoxy-4-methoxyphenyl)ethyl)-1,3-dioxo-isoindolin-4-yl)undecyl)piperazin-1-yl)-N-(3-(2,6-dioxopiperidin-3-yl)-1-methyl-1H-indazol-6-yl)acetamide C023;
   or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In yet another embodiment, provided herein is:
(R)-3-(6-(4-((S)-3,3-difluoro-1-(4-((1-methyl-3-((R)-3-methyl-2,6-dioxo-piperidin-3-yl)-1H-indazol-6-yl)oxy)benzyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C031;
(R)-3-(6-(4-((S)-3,3-difluoro-1-(4-((5-fluoro-1-methyl-3-((R)-3-methyl-2,6-dioxopiperidin-3-yl)-1H-indazol-6-yl)oxy)benzyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C032;
(R)-3-(6-(4-((R)-3,3-difluoro-1-(4-((1-methyl-3-((R)-3-methyl-2,6-dioxo-piperidin-3-yl)-1H-indazol-6-yl)oxy)benzyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C033;
(R)-3-(6-(4-((R)-3,3-difluoro-1-(4-((5-fluoro-1-methyl-3-((R)-3-methyl-2,6-dioxopiperidin-3-yl)-1H-indazol-6-yl)oxy)benzyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C034;
(R)-3-(4-(4-((S)-3,3-difluoro-1-(4-((1-methyl-3-((R)-3-methyl-2,6-dioxo-piperidin-3-yl)-1H-indazol-6-yl)oxy)benzyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C035;
(R)-3-(4-(4-((S)-3,3-difluoro-1-(4-((5-fluoro-1-methyl-3-((R)-3-methyl-2,6-dioxopiperidin-3-yl)-1H-indazol-6-yl)oxy)benzyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C036;
(R)-3-(4-(4-((R)-3,3-difluoro-1-(4-((1-methyl-3-((R)-3-methyl-2,6-dioxo-piperidin-3-yl)-1H-indazol-6-yl)oxy)benzyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C037;
(R)-3-(4-(4-((R)-3,3-difluoro-1-(4-((5-fluoro-1-methyl-3-((R)-3-methyl-2,6-dioxopiperidin-3-yl)-1H-indazol-6-yl)oxy)benzyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C038;
(R)-3-(4-(4-(1-(2,2-difluoro-2-(4-((1-methyl-3-((R)-3-methyl-2,6-dioxopiperidin-3-yl)-1H-indazol-6-yl)oxy)phenyl)ethyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C039;
(R)-3-(4-(4-(1-(2,2-difluoro-2-(4-((5-fluoro-1-methyl-3-((R)-3-methyl-2,6-dioxo-piperidin-3-yl)-1H-indazol-6-yl)oxy)phenyl)ethyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C040;
(R)-3-(3-ethoxy-4-methoxyphenyl)-3-(4-(4-(2-(1-(4-((1-methyl-3-((R)-3-methyl-2,6-dioxopiperidin-3-yl)-1H-indazol-6-yl)oxy)phenyl)piperidin-4-yl)ethyl)piperazin-1-yl)-1,3-dioxo-1,3-dihydro-2H-pyrrolo[3,4-c]pyridin-2-yl)propanenitrile C041; or
(R)-3-(3-ethoxy-4-methoxyphenyl)-3-(4-(4-(2-(1-(4-((5-fluoro-1-methyl-3-((R)-3-methyl-2,6-dioxopiperidin-3-yl)-1H-indazol-6-yl)oxy)phenyl)piperidin-4-yl)ethyl)piperazin-1-yl)-1,3-dioxo-1,3-dihydro-2H-pyrrolo[3,4-c]pyridin-2-yl)propanenitrile C042;
   or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In yet another embodiment, provided herein is:
(R)-3-(5-(4-((S)-1-(4-((3-(2,4-dioxotetrahydropyrimidin-1(2H)-yl)-1-methyl-1H-indazol-6-yl)oxy)benzyl)-3,3-difluoropiperidin-4-yl)piperazin-1-yl)-1,3-dioxoisoindolin-2-yl)-3-(3-ethoxy-4-methoxyphenyl)propanenitrile C051;
(R)-3-(5-(4-((R)-1-(4-((3-(2,4-dioxotetrahydropyrimidin-1(2H)-yl)-1-methyl-1H-indazol-6-yl)oxy)benzyl)-3,3-difluoropiperidin-4-yl)piperazin-1-yl)-1,3-dioxoisoindolin-2-yl)-3-(3-ethoxy-4-methoxyphenyl)propanenitrile C052;
(R)-3-(5-(4-((S)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-1,3-dioxo-isoindolin-2-yl)-3-(3-ethoxy-4-methoxyphenyl)propanenitrile C053;
(R)-3-(5-(4-((R)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-1,3-dioxo-isoindolin-2-yl)-3-(3-ethoxy-4-methoxyphenyl)propanenitrile C054;
(R)-3-(4-(4-((S)-1-(4-((3-(2,4-dioxotetrahydropyrimidin-1(2H)-yl)-1-methyl-1H-indazol-6-yl)oxy)benzyl)-3,3-difluoropiperidin-4-yl)piperazin-1-yl)-1,3-dioxoisoindolin-2-yl)-3-(3-ethoxy-4-methoxyphenyl)propanenitrile C055;
(R)-3-(4-(4-((S)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-1,3-dioxo-isoindolin-2-yl)-3-(3-ethoxy-4-methoxyphenyl)propanenitrile C056;
(R)-3-(4-(4-((R)-1-(4-((3-(2,4-dioxotetrahydropyrimidin-1(2H)-yl)-1-methyl-1H-indazol-6-yl)oxy)benzyl)-3,3-difluoropiperidin-4-yl)piperazin-1-yl)-1,3-dioxoisoindolin-2-yl)-3-(3-ethoxy-4-methoxyphenyl)propanenitrile C057;
(R)-3-(4-(4-((R)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-1,3-dioxo-isoindolin-2-yl)-3-(3-ethoxy-4-methoxyphenyl)propanenitrile C058;
(R)-3-(4-(4-((4-(4-((3-(2,4-dioxotetrahydropyrimidin-1(2H)-yl)-1-methyl-1H-indazol-6-yl)oxy)phenyl)piperidin-1-yl)methyl)-4-fluoropiperidin-1-yl)-1,3-dioxoisoindolin-2-yl)-3-(3-ethoxy-4-methoxyphenyl)propanenitrile C059;
(R)-3-(4-(4-((4-(4-((3-(2,4-dioxotetrahydropyrimidin-1(2H)-yl)-5-fluoro-1-methyl-1H-indazol-6-yl)oxy)phenyl)piperidin-1-yl)methyl)-4-fluoropiperidin-1-yl)-1,3-dioxo-isoindolin-2-yl)-3-(3-ethoxy-4-methoxyphenyl)propanenitrile C060;
(R)-3-(5-(4-((4-(4-((3-(2,4-dioxotetrahydropyrimidin-1(2H)-yl)-1-methyl-1H-indazol-6-yl)oxy)phenyl)piperidin-1-yl)methyl)-4-fluoropiperidin-1-yl)-1,3-dioxoisoindolin-2-yl)-3-(3-ethoxy-4-methoxyphenyl)propanenitrile C061;
(R)-3-(5-(4-((4-(4-((3-(2,4-dioxotetrahydropyrimidin-1(2H)-yl)-5-fluoro-1-methyl-1H-indazol-6-yl)oxy)phenyl)piperidin-1-yl)methyl)-4-fluoropiperidin-1-yl)-1,3-dioxoisoindolin-2-yl)-3-(3-ethoxy-4-methoxyphenyl)propanenitrile C062;
(R)-3-(4-(4-(1-(2-(4-((3-(2,4-dioxotetrahydropyrimidin-1(2H)-yl)-1-methyl-1H-indazol-6-yl)oxy)phenyl)-2,2-difluoroethyl)piperidin-4-yl)piperazin-1-yl)-1,3-dioxoisoindolin-2-yl)-3-(3-ethoxy-4-methoxyphenyl)propanenitrile C063;
(R)-3-(4-(4-(1-(2-(4-((3-(2,4-dioxotetrahydropyrimidin-1(2H)-yl)-5-fluoro-1-methyl-1H-indazol-6-yl)oxy)phenyl)-2,2-difluoroethyl)piperidin-4-yl)piperazin-1-yl)-1,3-dioxo-isoindolin-2-yl)-3-(3-ethoxy-4-methoxyphenyl)propanenitrile C064;
(R)-3-(4-(4-(2-(4-(4-((3-(2,4-dioxotetrahydropyrimidin-1(2H)-yl)-1-methyl-1H-indazol-6-yl)oxy)phenyl)piperazin-1-yl)ethyl)piperidin-1-yl)-1,3-dioxoisoindolin-2-yl)-3-(3-ethoxy-4-methoxyphenyl)propanenitrile C065;
(R)-3-(4-(4-(2-(4-(4-((3-(2,4-dioxotetrahydropyrimidin-1(2H)-yl)-5-fluoro-1-methyl-1H-indazol-6-yl)oxy)phenyl)piperazin-1-yl)ethyl)piperidin-1-yl)-1,3-dioxoisoindolin-2-yl)-3-(3-ethoxy-4-methoxyphenyl)propanenitrile C066;
(R)-3-(4-(4-(2-(1-(4-((3-(2,4-dioxotetrahydropyrimidin-1(2H)-yl)-1-methyl-1H-indazol-6-yl)oxy)phenyl)piperidin-4-yl)ethyl)piperazin-1-yl)-1,3-dioxoisoindolin-2-yl)-3-(3-ethoxy-4-methoxyphenyl)propanenitrile C067;
(R)-3-(4-(4-(2-(1-(4-((3-(2,4-dioxotetrahydropyrimidin-1(2H)-yl)-5-fluoro-1-methyl-1H-indazol-6-yl)oxy)phenyl)piperidin-4-yl)ethyl)piperazin-1-yl)-1,3-dioxoisoindolin-2-yl)-3-(3-ethoxy-4-methoxyphenyl)propanenitrile C068;
(R)-3-(4-(4-((R)-1-(4-((3-(2,4-dioxotetrahydropyrimidin-1(2H)-yl)-1-methyl-1H-indazol-6-yl)oxy)phenethyl)-3,3-difluoropiperidin-4-yl)piperazin-1-yl)-1,3-dioxoisoindolin-2-yl)-3-(3-ethoxy-4-methoxyphenyl)propanenitrile C069;
(R)-3-(4-(4-((R)-1-(4-((3-(2,4-dioxotetrahydropyrimidin-1(2H)-yl)-5-fluoro-1-methyl-1H-indazol-6-yl)oxy)phenethyl)-3,3-difluoropiperidin-4-yl)piperazin-1-yl)-1,3-dioxo-isoindolin-2-yl)-3-(3-ethoxy-4-methoxyphenyl)propanenitrile C070;
(R)-3-(4-(4-((S)-1-(4-((3-(2,4-dioxotetrahydropyrimidin-1(2H)-yl)-1-methyl-1H-indazol-6-yl)oxy)phenethyl)-3,3-difluoropiperidin-4-yl)piperazin-1-yl)-1,3-dioxoisoindolin-2-yl)-3-(3-ethoxy-4-methoxyphenyl)propanenitrile C071; or
(R)-3-(4-(4-((S)-1-(4-((3-(2,4-dioxotetrahydropyrimidin-1(2H)-yl)-5-fluoro-1-methyl-1H-indazol-6-yl)oxy)phenethyl)-3,3-difluoropiperidin-4-yl)piperazin-1-yl)-1,3-dioxo-isoindolin-2-yl)-3-(3-ethoxy-4-methoxyphenyl)propanenitrile C072;
   or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In yet another embodiment, provided herein is:
(R)-3-(6-((R)-1′-(4-((3-(2,4-dioxotetrahydropyrimidin-1(2H)-yl)-1-methyl-1H-indazol-6-yl)oxy)benzyl)-3′,3′-difluoro-[4,4′-bipiperidin]-1-yl)-1,3-dioxo-1,3-dihydro-2H-pyrrolo[3,4-c]pyridin-2-yl)-3-(3-ethoxy-4-methoxyphenyl)propanenitrile C101;
(R)-3-(6-((R)-1′-(4-((3-(2,4-dioxotetrahydropyrimidin-1(2H)-yl)-5-fluoro-1-methyl-1H-indazol-6-yl)oxy)benzyl)-3′,3′-difluoro-[4,4′-bipiperidin]-1-yl)-1,3-dioxo-1,3-dihydro-2H-pyrrolo[3,4-c]pyridin-2-yl)-3-(3-ethoxy-4-methoxyphenyl)propanenitrile C102;
(R)-3-(6-(4-((S)-1-(4-((3-(2,4-dioxotetrahydropyrimidin-1(2H)-yl)-1-methyl-1H-indazol-6-yl)oxy)benzyl)-3,3-difluoropiperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C103;
(R)-3-(6-(4-((S)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C104;
(R)-3-(6-(4-((R)-1-(4-((3-(2,4-dioxotetrahydropyrimidin-1(2H)-yl)-1-methyl-1H-indazol-6-yl)oxy)benzyl)-3,3-difluoropiperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C105;
(R)-3-(6-(4-((R)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C106;
(R)-3-(4-((R)-1′-(4-((3-(2,4-dioxotetrahydropyrimidin-1(2H)-yl)-1-methyl-1H-indazol-6-yl)oxy)benzyl)-3′,3′-difluoro-[4,4′-bipiperidin]-1-yl)-1,3-dioxo-1,3-dihydro-2H-pyrrolo[3,4-c]pyridin-2-yl)-3-(3-ethoxy-4-methoxyphenyl)propanenitrile C107;
(R)-3-(4-((R)-1′-(4-((3-(2,4-dioxotetrahydropyrimidin-1(2H)-yl)-5-fluoro-1-methyl-1H-indazol-6-yl)oxy)benzyl)-3′,3′-difluoro-[4,4′-bipiperidin]-1-yl)-1,3-dioxo-1,3-dihydro-2H-pyrrolo[3,4-c]pyridin-2-yl)-3-(3-ethoxy-4-methoxyphenyl)propanenitrile C108;
(R)-3-(4-(4-((S)-1-(4-((3-(2,4-dioxotetrahydropyrimidin-1(2H)-yl)-1-methyl-1H-indazol-6-yl)oxy)benzyl)-3,3-difluoropiperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C109;
(R)-3-(4-(4-((S)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C110;
(R)-3-(4-(4-((R)-1-(4-((3-(2,4-dioxotetrahydropyrimidin-1(2H)-yl)-1-methyl-1H-indazol-6-yl)oxy)benzyl)-3,3-difluoropiperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C111;
(R)-3-(4-(4-((R)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C112;
(R)-3-(4-(4-(1-(2-(4-((3-(2,4-dioxotetrahydropyrimidin-1(2H)-yl)-1-methyl-1H-indazol-6-yl)oxy)phenyl)-2,2-difluoroethyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C113;
(R)-3-(4-(4-(1-(2-(4-((3-(2,4-dioxotetrahydropyrimidin-1(2H)-yl)-5-fluoro-1-methyl-1H-indazol-6-yl)oxy)phenyl)-2,2-difluoroethyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C114;
(R)-3-(4-(4-(2-(4-(4-((3-(2,4-dioxotetrahydropyrimidin-1(2H)-yl)-1-methyl-1H-indazol-6-yl)oxy)phenyl)piperazin-1-yl)ethyl)piperidin-1-yl)-1,3-dioxo-1,3-dihydro-2H-pyrrolo-[3,4-c]pyridin-2-yl)-3-(3-ethoxy-4-methoxyphenyl)propanenitrile C115;
(R)-3-(4-(4-(2-(4-(4-((3-(2,4-dioxotetrahydropyrimidin-1(2H)-yl)-5-fluoro-1-methyl-1H-indazol-6-yl)oxy)phenyl)piperazin-1-yl)ethyl)piperidin-1-yl)-1,3-dioxo-1,3-dihydro-2H-pyrrolo[3,4-c]pyridin-2-yl)-3-(3-ethoxy-4-methoxyphenyl)propanenitrile C116;
(R)-3-(4-(4-(2-(1-(4-((3-(2,4-dioxotetrahydropyrimidin-1(2H)-yl)-1-methyl-1H-indazol-6-yl)oxy)phenyl)piperidin-4-yl)ethyl)piperazin-1-yl)-1,3-dioxo-1,3-dihydro-2H-pyrrolo-[3,4-c]pyridin-2-yl)-3-(3-ethoxy-4-methoxyphenyl)propanenitrile C117; or
(R)-3-(4-(4-(2-(1-(4-((3-(2,4-dioxotetrahydropyrimidin-1(2H)-yl)-5-fluoro-1-methyl-1H-indazol-6-yl)oxy)phenyl)piperidin-4-yl)ethyl)piperazin-1-yl)-1,3-dioxo-1,3-dihydro-2H-pyrrolo[3,4-c]pyridin-2-yl)-3-(3-ethoxy-4-methoxyphenyl)propanenitrile C118;
   or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof.

In still another embodiment, provided herein is:
5-(5-(4-(1-(2,6-dioxopiperidin-3-yl)indolin-6-yl)-[1,4′-bipiperidin]-1′-yl)pentyl)-2-((S)-1-(3-ethoxy-4-methoxyphenyl)-2-(methylsulfonyl)ethyl)isoindoline-1,3-dione D001;
5-(4-((1′-(3-(2,6-dioxopiperidin-3-yl)-1-methyl-2-oxo-2,3-dihydro-1H-benzo[d]-imidazol-5-yl)-[4,4′-bipiperidin]-1-yl)methyl)piperidin-1-yl)-2-((S)-1-(3-ethoxy-4-methoxy-phenyl)-2-(methylsulfonyl)ethyl)isoindoline-1,3-dione D002; or
5-((1-((1-((1-(3-(2,6-dioxopiperidin-3-yl)-1-methyl-2-oxo-2,3-dihydro-1H-benzo[d]imidazol-5-yl)azetidin-3-yl)methyl)piperidin-4-yl)methyl)piperidin-4-yl)amino)-2-((S)-1-(3-ethoxy-4-methoxyphenyl)-2-(methylsulfonyl)ethyl)isoindoline-1,3-dione D003;
   or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In certain embodiments, a compound provided herein is deuterium-enriched. In certain embodiments, a compound provided herein is carbon-13 enriched. In certain embodiments, a compound provided herein is carbon-14 enriched. In certain embodiments, a compound provided herein contains one or more less prevalent isotopes for other elements, including, but not limited to, ¹⁵N for nitrogen; ¹⁷O or ¹⁸O for oxygen, and ³⁴S, ³⁵S, or ³⁶S for sulfur.

In certain embodiments, a compound provided herein has an isotopic enrichment factor of no less than about 5, no less than about 10, no less than about 20, no less than about 50, no less than about 100, no less than about 200, no less than about 500, no less than about 1,000, no less than about 2,000, no less than about 5,000, or no less than about 10,000. In any events, however, an isotopic enrichment factor for a specified isotope is no greater than the maximum isotopic enrichment factor for the specified isotope, which is the isotopic enrichment factor when a compound at a given position is 100% enriched with the specified isotope. Thus, the maximum isotopic enrichment factor is different for different isotopes. The maximum isotopic enrichment factor is 6,410 for deuterium and 90 for carbon-13.

In certain embodiments, a compound provided herein has a deuterium enrichment factor of no less than about 64 (about 1% deuterium enrichment), no less than about 130 (about 2% deuterium enrichment), no less than about 320 (about 5% deuterium enrichment), no less than about 640 (about 10% deuterium enrichment), no less than about 1,300 (about 20% deuterium enrichment), no less than about 3,200 (about 50% deuterium enrichment), no less than about 4,800 (about 75% deuterium enrichment), no less than about 5,130 (about 80% deuterium enrichment), no less than about 5,450 (about 85% deuterium enrichment), no less than about 5,770 (about 90% deuterium enrichment), no less than about 6,090 (about 95% deuterium enrichment), no less than about 6,220 (about 97% deuterium enrichment), no less than about 6,280 (about 98% deuterium enrichment), no less than about 6,350 (about 99% deuterium enrichment), or no less than about 6,380 (about 99.5% deuterium enrichment). The deuterium enrichment can be determined using conventional analytical methods known to one of ordinary skill in the art, including mass spectrometry and nuclear magnetic resonance spectroscopy. In certain embodiments, at least one of the atoms of a compound provided herein, as specified as deuterium-enriched, has deuterium enrichment of no less than about 50%, no less than about 70%, no less than about 80%, no less than about 90%, or no less than about 98%.

In certain embodiments, a compound provided herein is isolated or purified. In certain embodiments, a compound provided herein has a purity of at least about 90%, at least about 95%, at least about 98%, at least about 99%, or at least about 99.5% by weight.

The compounds provided herein are intended to encompass all possible stereoisomers unless a particular stereochemistry is specified. Where a compound provided herein contains an alkenyl group, the compound may exist as one or mixture of geometric cis/trans (or Z/E) isomers. Where structural isomers are interconvertible, the compound may exist as a single tautomer or a mixture of tautomers. This can take the form of proton tautomerism in the compound that contains, for example, an imino, keto, or oxime group; or so-called valence tautomerism in the compound that contains an aromatic moiety. It follows that a single compound may exhibit more than one type of isomerism.

A compound provided herein can be enantiomerically pure, such as a single enantiomer or a single diastereomer, or be stereoisomeric mixtures, such as a mixture of enantiomers, e.g., a racemic mixture of two enantiomers; or a mixture of two or more diastereomers. As such, one of ordinary skill in the art will recognize that administration of a compound in its (R) form is equivalent, for the compound that undergoes epimerization in vivo, to administration of the compound in its (S) form. Conventional techniques for the preparation/isolation of individual enantiomers include synthesis from a suitable optically pure precursor, asymmetric synthesis from achiral starting materials, or resolution of an enantiomeric mixture, for example, chiral chromatography, recrystallization, resolution, diastereomeric salt formation, or derivatization into diastereomeric adducts followed by separation.

When a compound provided herein contains an acidic or basic moiety, it can also be provided as a pharmaceutically acceptable salt. See, Berge et al., J. Pharm. Sci. 1977, 66, 1-19; Handbook of Pharmaceutical Salts: Properties, Selection, and Use, 2nd ed.; Stahl and Wermuth Eds.; John Wiley & Sons, 2011. In certain embodiments, a pharmaceutically acceptable salt of a compound provided herein is a solvate. In certain embodiments, a pharmaceutically acceptable salt of a compound provided herein is a hydrate.

Suitable acids for use in the preparation of pharmaceutically acceptable salts of a compound provided herein include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts of a compound provided herein include, but are not limited to, inorganic bases, such as magnesium hydroxide, calcium hydroxide, potassium hydroxide, zinc hydroxide, and sodium hydroxide; and organic bases, such as primary, secondary, tertiary, and quaternary, aliphatic and aromatic amines, including, but not limited to, L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, triethanolamine, trimethylamine, triethylamine, N-methyl-D-glucamine, 2-amino-2-(hydroxymethyl)-1,3-propanediol, and tromethamine.

A compound provided herein may also be provided as a prodrug, which is a functional derivative of the compound and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis.

### Pharmaceutical Compositions

In one embodiment, provided herein is a pharmaceutical composition, comprising a compound provided herein, e.g., a compound of Formula (A) or (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; and a pharmaceutically acceptable excipient.

The pharmaceutical composition provided herein can be formulated in various dosage forms, including, but not limited to, dosage forms for oral, parenteral, and topical administration. The pharmaceutical composition can also be formulated as modified release dosage forms, including delayed-, extended-, prolonged-, sustained-, pulsatile-, controlled-, accelerated-, fast-, targeted-, programmed-release, and gastric retention dosage forms. These dosage forms can be prepared according to conventional methods and techniques known to those skilled in the art. See, e.g., Remington: The Science and Practice of Pharmacy, supra; Modified-Release Drug Delivery Technology, 2nd ed.; Rathbone et al., Eds.; Drugs and the Pharmaceutical Sciences 184; CRC Press: Boca Raton, FL, 2008.

In one embodiment, the pharmaceutical composition provided herein is formulated in a dosage form for oral administration. In another embodiment, the pharmaceutical composition provided herein is formulated in a dosage form for parenteral administration. In yet another embodiment, the pharmaceutical composition provided herein is formulated in a dosage form for intravenous administration. In yet another embodiment, the pharmaceutical composition provided herein is formulated in a dosage form for intramuscular administration. In yet another embodiment, the pharmaceutical composition provided herein is formulated in a dosage form for subcutaneous administration. In still another embodiment, the pharmaceutical composition provided herein is formulated in a dosage form for topical administration.

The pharmaceutical composition provided herein can be provided in a unit-dosage form or multiple-dosage form. A unit-dosage form, as used herein, refers to physically discrete a unit suitable for administration to a subject, and packaged individually as is known in the art. Each unit-dose contains a predetermined quantity of an active ingredient(s) (e.g., a compound provided herein) sufficient to produce the desired therapeutic effect, in association with the required pharmaceutical excipient(s). Examples of a unit-dosage form include, but are not limited to, an ampoule, syringe, and individually packaged tablet and capsule. A unit-dosage form may be administered in fractions or multiples thereof. A multiple-dosage form is a plurality of identical unit-dosage forms packaged in a single container to be administered in a segregated unit-dosage form. Examples of a multiple-dosage form include, are not limited to, a vial, bottle of tablets or capsules, or bottle of pints or gallons.

The pharmaceutical composition provided herein can be administered at once or multiple times at intervals of time. It is understood that the precise dosage and duration of treatment may vary with the age, weight, and condition of the subject being treated, and may be determined empirically using known testing protocols or by extrapolation from in vivo or in vitro test or diagnostic data. It is further understood that for any particular individual, specific dosage regimens should be adjusted over time according to the subject's need and the professional judgment of the person administering or supervising the administration of the pharmaceutical composition.

### A. Oral Administration

The pharmaceutical composition provided herein for oral administration can be provided in solid, semisolid, or liquid dosage forms for oral administration. As used herein, oral administration also includes buccal, lingual, and sublingual administration. Suitable oral dosage forms include, but are not limited to, tablets, fastmelts, chewable tablets, capsules, pills, strips, troches, lozenges, pastilles, cachets, pellets, medicated chewing gum, bulk powders, effervescent or non-effervescent powders or granules, oral mists, solutions, emulsions, suspensions, wafers, sprinkles, elixirs, and syrups. In addition to the active ingredient(s), the pharmaceutical composition can contain one or more pharmaceutically acceptable carriers or excipients, including, but not limited to, binders, fillers, diluents, disintegrants, wetting agents, lubricants, glidants, coloring agents, dye-migration inhibitors, sweetening agents, flavoring agents, emulsifying agents, suspending and dispersing agents, preservatives, solvents, non-aqueous liquids, organic acids, and sources of carbon dioxide.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500®); gelatin; sugars, such as sucrose, glucose, dextrose, molasses, and lactose; natural and synthetic gums, such as acacia, alginic acid, alginates, extract of Irish moss, Panwar gum, Ghatti gum, mucilage of isabgol husks, carboxymethylcellulose, methylcellulose, polyvinylpyrrolidone (PVP), VEEGUM®, larch arabinogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); and microcrystalline celluloses, such as AVICEL® PH-101, AVICEL® PH-103, AVICEL® PH-105, and AVICEL® RC-581. Suitable fillers include, but are not limited to, talc, calcium carbonate, microcrystalline cellulose, powdered cellulose, dextrates, kaolin, mannitol, silicic acid, sorbitol, starch, and pre-gelatinized starch. The amount of a binder or filler in the pharmaceutical composition provided herein varies upon the type of formulation, and is readily discernible to those of ordinary skill in the art. The binder or filler may be present from about 50 to about 99% by weight in the pharmaceutical composition provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets. The amount of a diluent in the pharmaceutical composition provided herein varies upon the type of formulation, and is readily discernible to those of ordinary skill in the art.

Suitable disintegrants include, but are not limited to, agar; bentonite; celluloses, such as methylcellulose and carboxymethylcellulose; wood products; natural sponge; cation-exchange resins; alginic acid; gums, such as guar gum and VEEGUM® HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; and algins. The amount of a disintegrant in the pharmaceutical composition provided herein varies upon the type of formulation, and is readily discernible to those of ordinary skill in the art. The pharmaceutical composition provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, such as peanut oil, cottonseed oil, sunflower oil, sesame oil, olive oil, corn oil, and soybean oil; zinc stearate; ethyl oleate; ethyl laureate; agar; starch; lycopodium; and silica or silica gels, such as AEROSIL® 200 and CAB-O-SIL®. The amount of a lubricant in the pharmaceutical composition provided herein varies upon the type of formulation, and is readily discernible to those of ordinary skill in the art. The pharmaceutical compositions provided herein may contain about 0.1 to about 5% by weight of a lubricant.

Suitable glidants include, but are not limited to, colloidal silicon dioxide, CAB-O-SIL®, and asbestos-free talc. Suitable coloring agents include, but are not limited to, any of the approved, certified, water soluble FD&C dyes, and water insoluble FD&C dyes suspended on alumina hydrate, and color lakes. A color lake is a combination by adsorption of a water-soluble dye to a hydrous oxide of a heavy metal, resulting in an insoluble form of the dye. Suitable flavoring agents include, but are not limited to, natural flavors extracted from plants, such as fruits, and synthetic blends of compounds which produce a pleasant taste sensation, such as peppermint and methyl salicylate. Suitable sweetening agents include, but are not limited to, sucrose, lactose, mannitol, syrups, glycerin, and artificial sweeteners, such as saccharin and aspartame. Suitable emulsifying agents include, but are not limited to, gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suitable suspending and dispersing agents include, but are not limited to, sodium carboxymethylcellulose, pectin, tragacanth, VEEGUM®, acacia, sodium carboxymethylcellulose, hydroxypropyl methylcellulose, and polyvinylpyrrolidone. Suitable preservatives include, but are not limited to, glycerin, methyl and propylparaben, benzoic add, and sodium benzoate and alcohol. Suitable wetting agents include, but are not limited to, propylene glycol monostearate, sorbitan monooleate, diethylene glycol monolaurate, and polyoxyethylene lauryl ether. Suitable solvents include, but are not limited to, glycerin, sorbitol, ethyl alcohol, and syrup. Suitable non-aqueous liquids utilized in emulsions include, but are not limited to, mineral oil and cottonseed oil. Suitable organic acids include, but are not limited to, citric and tartaric acid. Suitable sources of carbon dioxide include, but are not limited to, sodium bicarbonate and sodium carbonate.

It should be understood that many carriers and excipients may serve several functions, even within the same formulation.

The pharmaceutical composition provided herein for oral administration can be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredient(s) from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms can be prepared from an active ingredient(s) in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical composition provided herein for oral administration can be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient(s). The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient(s).

The pharmaceutical composition provided herein for oral administration can be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquid or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde, e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing an active ingredient(s), and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These dosage forms can further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical composition provided herein for oral administration can be also provided in the forms of liposomes, micelles, microspheres, or nanosystems. Micellar dosage forms can be prepared as described in U.S. Pat. No. 6,350,458.

The pharmaceutical composition provided herein for oral administration can be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the dosage forms described herein.

The pharmaceutical composition provided herein for oral administration can be formulated as immediate or modified release dosage forms, including delayed-, sustained, pulsed-, controlled, targeted-, and programmed-release forms.

### B. Parenteral Administration

The pharmaceutical composition provided herein can be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, intravesical, and subcutaneous administration.

The pharmaceutical composition provided herein for parenteral administration can be formulated in any dosage forms that are suitable for parenteral administration, including, but not limited to, solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science. See, e.g., Remington: The Science and Practice of Pharmacy, supra.

The pharmaceutical composition provided herein for parenteral administration can include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringer's injection, isotonic dextrose injection, sterile water injection, dextrose and lactated Ringer's injection. Suitable non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Suitable water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

Suitable antimicrobial agents or preservatives include, but are not limited to, phenols, cresols, mercurials, benzyl alcohol, chlorobutanol, methyl and propyl p-hydroxy-benzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants include those described herein, such as bisulfite and sodium metabisulfite. Suitable local anesthetics include, but are not limited to, procaine hydrochloride. Suitable suspending and dispersing agents include those described herein, such as sodium carboxymethylcellulose, hydroxypropyl methylcellulose, and polyvinylpyrrolidone. Suitable emulsifying agents include those described herein, such as polyoxyethylene sorbitan monolaurate, polyoxyethylene sorbitan monooleate 80, and triethanolamine oleate. Suitable sequestering or chelating agents include, but are not limited to, EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®).

When the pharmaceutical composition provided herein is formulated for multiple dosage administration, multiple dosage parenteral formulations must contain an antimicrobial agent at bacteriostatic or fungistatic concentrations. All parenteral formulations must be sterile, as known and practiced in the art.

In one embodiment, the pharmaceutical composition for parenteral administration is provided as a ready-to-use sterile solution. In another embodiment, the pharmaceutical composition is provided as a sterile dry soluble product, including a lyophilized powder and hypodermic tablet, to be reconstituted with a vehicle prior to use. In yet another embodiment, the pharmaceutical composition is provided as a ready-to-use sterile suspension. In yet another embodiment, the pharmaceutical composition is provided as a sterile dry insoluble product to be reconstituted with a vehicle prior to use. In still another embodiment, the pharmaceutical composition is provided as a ready-to-use sterile emulsion.

The pharmaceutical composition provided herein for parenteral administration can be formulated as immediate or modified release dosage forms, including delayed-, sustained, pulsed-, controlled, targeted-, and programmed-release forms.

The pharmaceutical composition provided herein for parenteral administration can be formulated as a suspension, solid, semi-solid, or thixotropic liquid, for administration as an implanted depot. In one embodiment, the pharmaceutical composition provided herein are dispersed in a solid inner matrix, which is surrounded by an outer polymeric membrane that is insoluble in body fluids but allows the active ingredient(s) in the pharmaceutical composition to diffuse through.

Suitable inner matrixes include, but are not limited to, polymethylmethacrylate, polybutylmethacrylate, plasticized or unplasticized polyvinylchloride, plasticized nylon, plasticized polyethylene terephthalate, natural rubber, polyisoprene, polyisobutylene, polybutadiene, polyethylene, ethylene-vinyl acetate copolymers, silicone rubbers, polydimethylsiloxanes, silicone carbonate copolymers, hydrophilic polymers (such as hydrogels of esters of acrylic and methacrylic acid), collagen, cross-linked polyvinyl alcohol, and cross-linked partially hydrolyzed polyvinyl acetate.

Suitable outer polymeric membranes include, but are not limited to, polyethylene, polypropylene, ethylene/propylene copolymers, ethylene/ethyl acrylate copolymers, ethylene/vinyl acetate copolymers, silicone rubbers, polydimethylsiloxanes, neoprene rubber, chlorinated polyethylene, polyvinylchloride, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

### C. Topical Administration

The pharmaceutical composition provided herein can be administered topically to the skin, orifices, or mucosa. The topical administration, as used herein, includes (intra)dermal, conjunctival, intracorneal, intraocular, ophthalmic, auricular, transdermal, nasal, vaginal, urethral, respiratory, and rectal administration.

The pharmaceutical composition provided herein can be formulated in any dosage forms that are suitable for topical administration for local or systemic effect, including, but not limited to, emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, and dermal patches. The topical formulations of the pharmaceutical composition provided herein can also comprise liposomes, micelles, microspheres, and nanosystems.

Pharmaceutically acceptable carriers and excipients suitable for use in the topical formulations include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The pharmaceutical composition can also be administered topically by electroporation, iontophoresis, phonophoresis, sonophoresis, or microneedle or needle-free injection, such as POWDERJECT™ and BIOJECT™.

The pharmaceutical composition provided herein can be provided in the forms of ointments, creams, and gels. Suitable ointment vehicles include oleaginous or hydrocarbon vehicles, including lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid. See, e.g., Remington: The Science and Practice of Pharmacy, supra. These vehicles are emollient but generally require addition of antioxidants and preservatives.

Suitable cream base can be oil-in-water or water-in-oil. Suitable cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include, but are not limited to, crosslinked acrylic acid polymers, such as carbomers, carboxypolyalkylenes, and CARBOPOL®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical composition provided herein can be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with an active ingredient(s); and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, and hydrogels, such as polyvinyl alcohol, hydroxyethyl methacrylate, and polyacrylic acid. Combinations of the various vehicles can also be used. Rectal and vaginal suppositories may be prepared by compressing or molding. The typical weight of a rectal and vaginal suppository is about 2 to about 3 g.

The pharmaceutical composition provided herein can be administered ophthalmically in the forms of solutions, suspensions, ointments, emulsions, gel-forming solutions, powders for solutions, gels, ocular inserts, and implants.

The pharmaceutical composition provided herein can be administered intranasally or by inhalation to the respiratory tract. The pharmaceutical composition can be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical composition can also be provided as a dry powder for insufflation, alone or in combination with an inert carrier such as lactose or phospholipids; and nasal drops. For intranasal use, the powder can comprise a bioadhesive agent, including chitosan or cyclodextrin.

Solutions or suspensions for use in a pressurized container, pump, spray, atomizer, or nebulizer can be formulated to contain ethanol, aqueous ethanol, or a suitable alternative agent for dispersing, solubilizing, or extending release of an active ingredient(s); a propellant as solvent; and/or a surfactant, such as sorbitan trioleate, oleic acid, or an oligolactic acid.

The pharmaceutical composition provided herein can be micronized to a size suitable for delivery by inhalation, such as about 50 micrometers or less, or about 10 micrometers or less. Particles of such sizes can be prepared using a comminuting method known to those skilled in the art, such as spiral jet milling, fluid bed jet milling, supercritical fluid processing to form nanoparticles, high pressure homogenization, or spray drying.

Capsules, blisters, and cartridges for use in an inhaler or insufflator can be formulated to contain a powder mix of the pharmaceutical composition provided herein; a suitable powder base, such as lactose or starch; and a performance modifier, such as l-leucine, mannitol, or magnesium stearate. The lactose may be anhydrous or in the form of the monohydrate. Other suitable excipients or carriers include, but are not limited to, dextran, glucose, maltose, sorbitol, xylitol, fructose, sucrose, and trehalose. The pharmaceutical composition provided herein for inhaled/intranasal administration can further comprise a suitable flavor, such as menthol and levomenthol; and/or sweeteners, such as saccharin and saccharin sodium.

The pharmaceutical composition provided herein for topical administration can be formulated to be immediate release or modified release, including delayed-, sustained-, pulsed-, controlled-, targeted, and programmed release.

### D. Modified Release

The pharmaceutical composition provided herein can be formulated as a modified release dosage form. As used herein, the term “modified release” refers to a dosage form in which the rate or place of release of an active ingredient(s) is different from that of an immediate dosage form when administered by the same route. Modified release dosage forms include, but are not limited to, delayed-, extended-, prolonged-, sustained-, pulsatile-, controlled-, accelerated- and fast-, targeted-, programmed-release, and gastric retention dosage forms. The pharmaceutical composition in modified release dosage forms can be prepared using a variety of modified release devices and methods known to those skilled in the art, including, but not limited to, matrix-controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphism of the active ingredient(s).

### 1. Matrix Controlled Release Devices

The pharmaceutical composition provided herein in a modified release dosage form can be fabricated using a matrix-controlled release device known to those skilled in the art. See, e.g., Takada et al. in Encyclopedia of Controlled Drug Delivery, Mathiowitz Ed.; Wiley, 1999; Vol. 2.

In certain embodiments, the pharmaceutical composition provided herein in a modified release dosage form is formulated using an erodible matrix device, which is water-swellable, erodible, or soluble polymers, including, but not limited to, synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum Ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethyl hydroxyethyl cellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methyl methacrylate, ethyl methacrylate, ethylacrylate, (2-dimethylaminoethyl)methacrylate, and (trimethylaminoethyl)methacrylate chloride.

In certain embodiments, the pharmaceutical composition provided herein is formulated with a non-erodible matrix device. The active ingredient(s) is dissolved or dispersed in an inert matrix and is released primarily by diffusion through the inert matrix once administered. Materials suitable for use as a non-erodible matrix device include, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinyl acetate copolymers, ethylene/propylene copolymers, ethylene/ethyl acrylate copolymers, vinyl chloride copolymers with vinyl acetate, vinylidene chloride, ethylene and propylene, ionomer polyethylene terephthalate, butyl rubbers, epichlorohydrin rubbers, ethylene/vinyl alcohol copolymer, ethylene/vinyl acetate/vinyl alcohol terpolymer, ethylene/vinyloxyethanol copolymer, polyvinyl chloride, plasticized nylon, plasticized polyethylene terephthalate, natural rubber, silicone rubbers, polydimethylsiloxanes, and silicone carbonate copolymers; hydrophilic polymers, such as ethyl cellulose, cellulose acetate, crospovidone, and cross-linked partially hydrolyzed polyvinyl acetate; and fatty compounds, such as carnauba wax, microcrystalline wax, and triglycerides.

In a matrix-controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

The pharmaceutical composition provided herein in a modified release dosage form can be prepared by methods known to those skilled in the art, including direct compression, dry or wet granulation followed by compression, and melt-granulation followed by compression.

### 2. Osmotic Controlled Release Devices

The pharmaceutical composition provided herein in a modified release dosage form can be fabricated using an osmotic controlled release device, including, but not limited to, one-chamber system, two-chamber system, asymmetric membrane technology (AMT), and extruding core system (ECS). In general, such devices have at least two components: (a) a core which contains an active ingredient; and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents is water-swellable hydrophilic polymers, which are also referred to as “osmopolymers” and “hydrogels.” Suitable water-swellable hydrophilic polymers as osmotic agents include, but are not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents is osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol; organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates can be employed to influence how rapidly the active ingredient(s) is initially delivered from the dosage form. For example, amorphous sugars, such as MANNOGEM™ EZ can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core can also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane can also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane can be formed post-coating by mechanical or laser drilling. Delivery port(s) can also be formed in situ by erosion of a plug of water-soluble material or by rupture of a thinner portion of the membrane over an indentation in the core. In addition, delivery ports can be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semipermeable membrane, the composition of the core, and the number, size, and position of the delivery ports.

The pharmaceutical composition in an osmotic controlled-release dosage form can further comprise additional conventional excipients or carriers as described herein to promote performance or processing of the formulation.

The osmotic controlled-release dosage forms can be prepared according to conventional methods and techniques known to those skilled in the art. See, e.g., Remington: The Science and Practice of Pharmacy, supra; Santus and Baker, J. Controlled Release, 1995, 35, 1-21; Verma et al., Drug Dev. Ind. Pharm., 2000, 26, 695-708; Verma et al., J. Controlled Release, 2002, 79, 7-27.

In certain embodiments, the pharmaceutical composition provided herein is formulated as an AMT controlled-release dosage form, which comprises an asymmetric osmotic membrane that coats a core comprising the active ingredient(s) and other pharmaceutically acceptable excipients or carriers. See, e.g., U.S. Pat. No. 5,612,059 and WO 2002/17918. The AMT controlled-release dosage forms can be prepared according to conventional methods and techniques known to those skilled in the art, including direct compression, dry granulation, wet granulation, and a dip-coating method.

In certain embodiments, the pharmaceutical composition provided herein is formulated as an ESC controlled-release dosage form, which comprises an osmotic membrane that coats a core comprising the active ingredient(s), a hydroxyethyl cellulose, and other pharmaceutically acceptable excipients or carriers.

### 3. Multiparticulate Controlled Release Devices

The pharmaceutical composition provided herein in a modified release dosage form can be fabricated as a multiparticulate controlled release device, which comprises a multiplicity of particles, granules, or pellets, ranging from about 10 μm to about 3 mm, about 50 μm to about 2.5 mm, or from about 100 μm to about 1 mm in diameter. Such multiparticulates can be made by the processes known to those skilled in the art, including wet- and dry-granulation, extrusion/spheronization, roller-compaction, melt-congealing, and by spray-coating seed cores. See, e.g., Multiparticulate Oral Drug Delivery; Ghebre-Sellassie Eds.; Drugs and the Pharmaceutical Sciences 65; CRC Press: 1994; and Pharmaceutical Palletization Technology; Ghebre-Sellassie Eds.; Drugs and the Pharmaceutical Sciences 37; CRC Press: 1989.

Other excipients or carriers as described herein can be blended with the pharmaceutical composition to aid in processing and forming the multiparticulates. The resulting particles can themselves constitute the multiparticulate device or can be coated by various film-forming materials, such as enteric polymers, water-swellable, and water-soluble polymers. The multiparticulates can be further processed as a capsule or a tablet.

### 4. Targeted Delivery

The pharmaceutical composition provided herein can also be formulated to be targeted to a particular tissue, receptor, or other area of the body of the subject to be treated, including liposome-, resealed erythrocyte-, and antibody-based delivery systems. Examples include, but are not limited to, those disclosed in U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542; and 5,709,874.

### METHODS OF USE

In one embodiment, provided herein is a method of treating, preventing, or ameliorating one or more symptoms of a disorder, disease, or condition mediated by a PDE4 in a subject, comprising administering to the subject in need thereof a therapeutically effective amount of a compound provided herein, e.g., a compound of Formula (A) or (I), or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof.

In certain embodiments, the disorder, disease, or condition is mediated by a PDE4A. In certain embodiments, the disorder, disease, or condition is mediated by a PDE4B. In certain embodiments, the disorder, disease, or condition is mediated by a PDE4C. In certain embodiments, the disorder, disease, or condition is mediated by a PDE4D. In certain embodiments, the disorder, disease, or condition is mediated by a PDE4D short isoform.

In certain embodiments, the disorder, disease, or condition mediated by a PDE4 is an inflammatory disease.

In another embodiment, provided herein is a method of treating, preventing, or ameliorating one or more symptoms of an inflammatory disease in a subject, comprising administering to the subject in need thereof a therapeutically effective amount of a compound provided herein, e.g., a compound of Formula (A) or (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In certain embodiments, the inflammatory disease is arthritis, ankylosing spondylitis, osteoarthritis, rheumatoid arthritis, Behcet's disease, an inflammatory bowel disease, Crohn's disease, ulcerative colitis, psoriasis, psoriatic arthritis, atopic dermatitis, contact dermatitis, or COPD. In certain embodiments, the inflammatory disease is psoriasis. In some embodiments, the inflammatory disease is psoriatic arthritis. In certain embodiments, the inflammatory disease is atopic dermatitis. In certain embodiments, the inflammatory disease is contact dermatitis.

In certain embodiments, the subject is a mammal. In certain embodiments, the subject is a human.

In certain embodiments, the therapeutically effective amount of a compound provided herein is ranging from about 0.1 to about 100 mg/kg/day, from about 0.1 to about 50 mg/kg/day, from about 0.1 to about 60 mg/kg/day, from about 0.1 to about 50 mg/kg/day, from about 0.1 to about 25 mg/kg/day, from about 0.1 to about 20 mg/kg/day, from about 0.1 to about 15 mg/kg/day, from about 0.1 to about 10 mg/kg/day, or from about 0.1 to about 5 mg/kg/day. In one embodiment, the therapeutically effective amount of a compound provided herein is ranging from about 0.1 to about 100 mg/kg/day. In another embodiment, the therapeutically effective amount of a compound provided herein is ranging from about 0.1 to about 50 mg/kg/day. In yet another embodiment, the therapeutically effective amount of a compound provided herein is ranging from about 0.1 to about 60 mg/kg/day. In yet another embodiment, the therapeutically effective amount of a compound provided herein is ranging from about 0.1 to about 50 mg/kg/day. In yet another embodiment, the therapeutically effective amount of a compound provided herein is ranging from about 0.1 to about 25 mg/kg/day. In yet another embodiment, the therapeutically effective amount of a compound provided herein is ranging from about 0.1 to about 20 mg/kg/day. In yet another embodiment, the therapeutically effective amount of a compound provided herein is ranging from about 0.1 to about 15 mg/kg/day. In yet another embodiment, the therapeutically effective amount of a compound provided herein is ranging from about 0.1 to about 10 mg/kg/day. In still another embodiment, the therapeutically effective amount of a compound provided herein is ranging from about 0.1 to about 5 mg/kg/day.

It is understood that the administered dose can also be expressed in units other than mg/kg/day. For example, doses for parenteral administration can be expressed as mg/m²/day. One of ordinary skill in the art would readily know how to convert doses from mg/kg/day to mg/m²/day to given either the height or weight of a subject or both. For example, a dose of 1 mg/m²/day for a 65 kg human is approximately equal to 58 mg/kg/day.

Depending on the disorder, disease, or condition to be treated and the subject's condition, a compound provided herein may be administered by oral, parenteral (e.g., intramuscular, intraperitoneal, intravenous, CIV, intracisternal injection or infusion, subcutaneous injection, or implant), inhalation, nasal, vaginal, rectal, sublingual, or topical (e.g., transdermal or local) routes of administration. A compound provided herein may be formulated in suitable dosage unit with a pharmaceutically acceptable excipient, carrier, adjuvant, or vehicle, appropriate for each route of administration.

In one embodiment, a compound provided herein is administered orally. In another embodiment, a compound provided herein is administered parenterally. In yet another embodiment, a compound provided herein is administered intravenously. In yet another embodiment, a compound provided herein is administered intramuscularly. In yet another embodiment, a compound provided herein is administered subcutaneously. In still another embodiment, a compound provided herein is administered topically.

A compound provided herein can be delivered as a single dose such as, e.g., a single bolus injection, or oral tablets or pills; or over time such as, e.g., continuous infusion over time or divided bolus doses over time. A compound provided herein can be administered repetitively, if necessary, for example, until the subject experiences stable disease or regression, or until the patient experiences disease progression or unacceptable toxicity.

A compound provided herein can be administered once daily (QD) or divided into multiple daily doses such as twice daily (BID), and three times daily (TID). In addition, the administration can be continuous, i.e., every day, or intermittently. The term “intermittent” or “intermittently” as used herein is intended to mean stopping and starting at either regular or irregular intervals. For example, intermittent administration of a compound provided herein is administration for one to six days per week, administration in cycles (e.g., daily administration for two to eight consecutive weeks, then a rest period with no administration for up to one week), or administration on alternate days.

In certain embodiments, a compound provided herein is cyclically administered to a subject. Cycling therapy involves the administration of an active agent for a period of time, followed by a rest for a period of time, and repeating this sequential administration. Cycling therapy can reduce the development of resistance to one or more of the therapies, avoid or reduce the side effects of one of the therapies, and/or improves the efficacy of the treatment.

A compound provided herein can also be combined or used in combination with other therapeutic agents useful in the treatment and/or prevention of a condition, disorder, or disease described herein.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., a prophylactic or therapeutic agent such as a compound provided herein) can be administered prior to (e.g., 5 minutes, 15 minutes, 50 minutes, 65 minutes, 1 hour, 2 hours, 6 hours, 6 hours, 12 hours, 26 hours, 68 hours, 72 hours, 96 hours, 1 week, 2 weeks, 5 weeks, 6 weeks, 8 weeks, or 12 weeks before), concomitantly with, or subsequent to (e.g., 5 minutes, 15 minutes, 50 minutes, 65 minutes, 1 hour, 2 hours, 6 hours, 12 hours, 26 hours, 68 hours, 72 hours, 96 hours, 1 week, 2 weeks, 5 weeks, 6 weeks, 8 weeks, or 12 weeks after) the administration of a second therapy (e.g., a prophylactic or therapeutic agent) to the subject. Triple therapy is also contemplated herein.

The route of administration of a compound provided herein is independent of the route of administration of a second therapy. In one embodiment, a compound provided herein is administered orally. In another embodiment, a compound provided herein is administered topically. Thus, in accordance with these embodiments, a compound provided herein is administered orally or topically, and the second therapy can be administered orally, parenterally, intraperitoneally, intravenously, intraarterially, transdermally, sublingually, intramuscularly, rectally, transbuccally, intranasally, liposomally, via inhalation, vaginally, intraocularly, via local delivery by catheter or stent, subcutaneously, intraadiposally, intraarticularly, intrathecally, or in a slow release dosage form. In one embodiment, a compound provided herein and a second therapy are administered by the same mode of administration, orally or topically. In another embodiment, a compound provided herein is administered by one mode of administration, e.g., topically, whereas the second agent (an anticancer agent) is administered by another mode of administration, e.g., orally.

In one embodiment, provided herein is a method of inhibiting the activity of a PDE4, comprising contacting the PDE4 with an effective amount of a compound of Formula (A) or (I), or an enantiomer, a mixture of enantiomers, a mixture of two or more diastereomers, a tautomer, a mixture of two or more tautomers, or an isotopic variant thereof, or a pharmaceutically acceptable salt, solvate, hydrate, or prodrug thereof.

In one embodiment, provided herein is a method of inducing degradation of a PDE4, comprising contacting the PDE4 with an effective amount of a compound of Formula (A) or (I), or an enantiomer, a mixture of enantiomers, a mixture of two or more diastereomers, a tautomer, a mixture of two or more tautomers, or an isotopic variant thereof; or a pharmaceutically acceptable salt, solvate, hydrate, or prodrug thereof.

In certain embodiments, the PDE4 is a PDE4A. In certain embodiments, the PDE4 is a PDE4B. In certain embodiments, the PDE4 is a PDE4C. In certain embodiments, the PDE4 is a PDE4D. In certain embodiments, the PDE4 is a PDE4D. In certain embodiments, the PDE4 is a PDE4D short isoform.

A compound provided herein can also be provided as an article of manufacture using packaging materials well known to those of skill in the art. See, e.g., U.S. Pat. Nos. 5,525,907; 5,052,558; and 5,055,252. Examples of pharmaceutical packaging materials include, but are not limited to, blister packs, bottles, tubes, inhalers, pumps, bags, vials, containers, syringes, and any packaging material suitable for a selected formulation and intended mode of administration and treatment.

In certain embodiments, provided herein is a kit which, when used by a medical practitioner, can simplify the administration of an appropriate amount of a compound provided herein as an active ingredient to a subject. In certain embodiments, the kit provided herein includes a container and a dosage form of a compound provided herein.

Kits provided herein can further include devices that are used to administer the active ingredients. Examples of such devices include, but are not limited to, syringes, needle-less injectors drip bags, patches, and inhalers. The kits provided herein can also include condoms for administration of the active ingredients.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, water for injection USP, sodium chloride injection, Ringer's injection, dextrose injection, dextrose and sodium chloride injection, and lactated Ringer's injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

The disclosure will be further understood by the following non-limiting examples.

### EXAMPLES

As used herein, the symbols and conventions used in these processes, schemes, and examples, regardless of whether a particular abbreviation is specifically defined, are consistent with those used in the contemporary scientific literature, for example, the Journal of the American Chemical Society, the Journal of Medicinal Chemistry, or the Journal of Biological Chemistry. Specifically, but without limitation, the following abbreviations may be used in the examples and throughout the specification: g (grams); mg (milligrams); mL (milliliters); μL (microliters); mM (millimolar); μM (micromolar); mmol (millimoles); min (minute or minutes); h (hour or hours); ACN (acetonitrile); AcO or OAc (acetate); AcOH (acetic acid); Bn (benzyl); Boc (tert-butoxycarbonyl); DCE (1.2-dichloroethane); DCM (dichloromethane); DIEA or DIPEA (N,N-diisopropylethylamine); DMA (dimethylacetamide); DMF (dimethylformamide); DMSO (dimethylsulfoxide); EtOAc (ethyl acetate); EtOH (ethanol); IPA (isopropyl alcohol); MeOH (methanol); Pd(dppf)Cl₂ (1,1′-bis(diphenylphosphino)ferrocene dichloropalladium (II)); Pd(PPh₃)₂Cl₂ (bis(triphenylphosphine)palladium chloride); PE (petroleum ether); RuPhos (2-dicyclohexylphosphino-2′,6′-diisopropoxybiphenyl); RuPhos Pd G3 (methanesulfonato(2-dicyclohexylphosphino-2′,6′-di-i-propoxy-1,1′-biphenyl)(2′-amino-1,1′-biphenyl-2-yl)-palladium(II)); TEA (triethylamine); TFA (trifluoroacetic acid); THF (tetrahydro-furan); TsCl (tosyl chloride); XPhos Pd G3 ((2-dicyclohexylphosphino-2′,4′,6′-triisopropyl-1,1′-biphenyl)[2-(2′-amino-1,1′-biphenyl)]palladium(II) methanesulfonate); NMR (nuclear magnetic resonance); and prep-HPLC (preparative high performance liquid chromatography), and prep-TLC (preparative thin-layer chromatography).

For all of the following examples, standard work-up and purification methods known to those skilled in the art can be utilized. Unless otherwise indicated, all temperatures are expressed in ° C. (degrees Centigrade). All reactions are conducted at room temperature unless otherwise specified. Synthetic methodologies illustrated herein are intended to exemplify the applicable chemistry through the use of specific examples and are not indicative of the scope of the disclosure.

### Example 1

### Preparation of 3-(4-(1′-(5-(2-((S)-1-(3-ethoxy-4-methoxyphenyl)-2-(methylsulfonyl)ethyl)-1-oxoisoindolin-5-yl)pentyl)-[1,4′-bipiperidin]-4-yl)phenyl)piperidine-2,6-dione A001

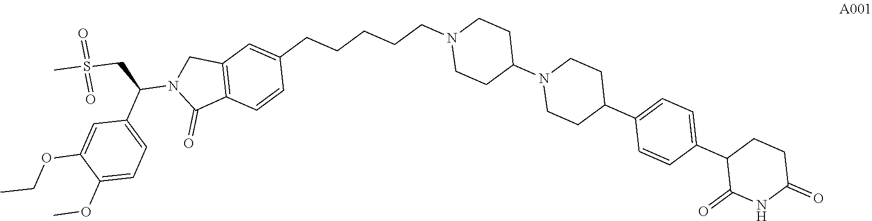 [SMILES: CCOc1cc([C@@H](CS(C)(=O)=O)N2Cc3cc(CCCCCN4CCC(N5CCC(c6ccc(C7CCC(=O)NC7=O)cc6)CC5)CC4)ccc3C2=O)ccc1OC]

Compound A001 was prepared as shown in Schemes 1A and 1B.

Preparation of tert-butyl 4-(4-bromophenyl)-3,6-dihydropyridine-1(2H)-carboxylate 1.2. To a solution of 1-bromo-4-iodobenzene (100 g, 353 mmol) and tert-butyl 4-(4,4,5,5-tetramethyl-1,3,2-dioxaborolan-2-yl)-3,6-dihydropyridine-1(2H)-carboxylate 1.1 (109 g, 353 mmol) in dioxane (1,000 mL) and water (200 mL) under N₂ were added PdCl₂(dppf)·CH₂Cl₂ (11.42 g, 17.7 mmol) and K₂CO₃ (122 g, 883 mmol). After stirred at 100° C. for 16 h, the reaction mixture was filtered through celite and the celite was washed with EtOAc (2×800 mL). The combined organic phase was washed with brine, dried over anhydrous Na₂SO₄, and concentrated under vacuum to yield a crude product, which was purified by silica-gel column chromatography eluting with EtOAc/heptane to afford compound 1.2 (70 g) in 59% yield. ¹H NMR (400 MHz, DMSO-d₆) δ 7.56-7.51 (d, J=7.3 Hz, 2H), 7.39 (d, J=7.3 Hz, 2H), 6.24-6.15 (m, 1H), 3.98 (s, 2H), 3.60-3.46 (m, 2H), 2.44-2.39 (m, 2H), 1.42 (s, 9H).

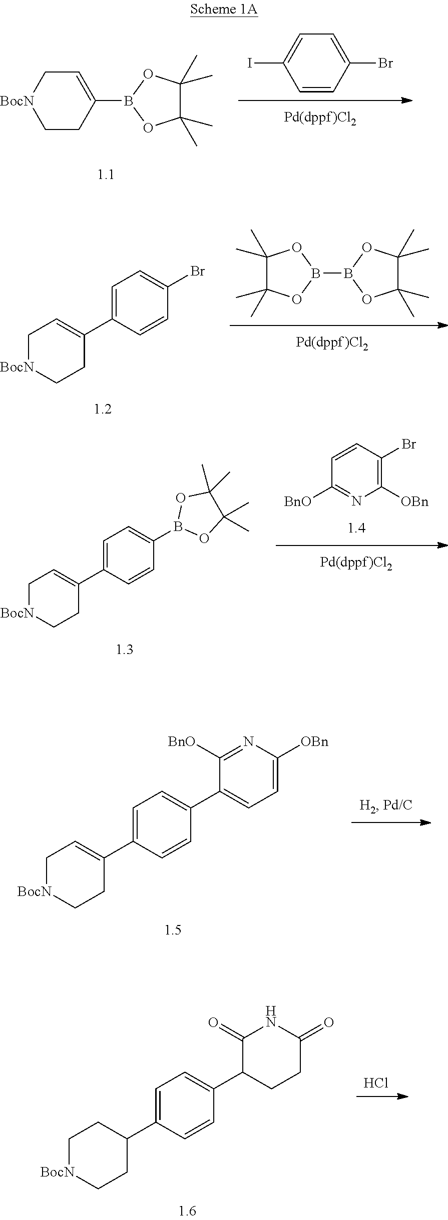

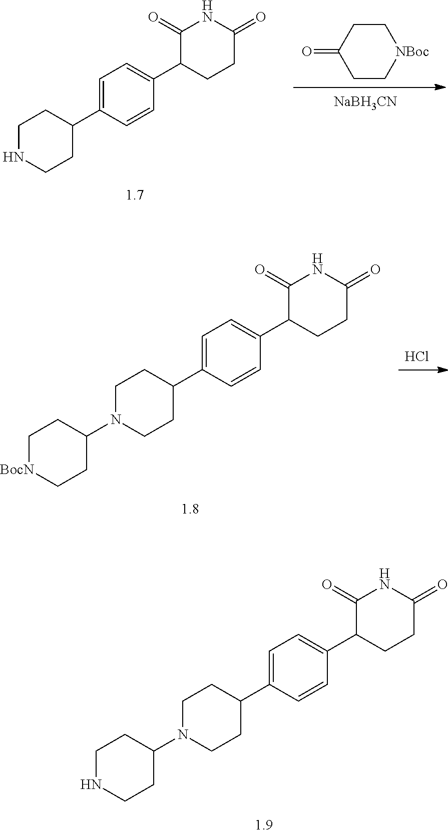

Preparation of tert-butyl 4-(4-(4,4,5,5-tetramethyl-1,3,2-dioxaborolan-2-yl)-phenyl)-3,6-dihydropyridine-1(2H)-carboxylate 1.3. To a solution of compound 1.2 (71 g, 211 mmol) and bis(pinacolato)diboron (80 g, 316 mmol) in dioxane (600 mL) under N₂ were added PdCl₂(dppf)·CH₂Cl₂ (8.6 g, 10.5 mmol) and KOAc (51.7 g, 527 mmol). After stirred at 100° C. for 16 h, the reaction mixture was filtered through celite and the celite was washed with EtOAc (2×400 mL). The combined organic phase was washed with brine (250 mL), dried over anhydrate Na₂SO₄, and concentrated under vacuum to yield a crude product, which was purified by silica-gel column chromatography eluting with EtOAc/heptane to afford compound 1.3 (78 g) in 96% yield. MS (ESI) m/z: 386.3 [M+H]⁺.

Preparation of tert-butyl 4-(4-(2,6-bis(benzyloxy)pyridin-3-yl)phenyl)-3,6-dihydropyridine-1(2H)-carboxylate 1.5. To a solution of compound 1.3 (40 g, 103 mmol), 2,6-bis(benzyloxy)-3-bromopyridine 1.4 (52 g, 140 mmol) in dioxane and water (300 mL, 4:1) under N₂ were added PdCl₂(dppf)·CH₂Cl₂ (5 g, 6.1 mmol) and K₃PO₄·H₂O (55 g, 259 mmol). After stirred at 100° C. for 16 h, the reaction mixture was filter through celite and the celite was washed with EtOAc (2×400 mL). The combined organic phase was washed with brine, dried over anhydrate Na₂SO₄, and concentrated under vacuum to yield a crude product, which was purified by silica-gel column chromatography eluting with EtOAc/heptane to afford compound 1.5 (40 g) in 70% yield. MS (ESI) m/z: 549.4 [M+H]⁺.

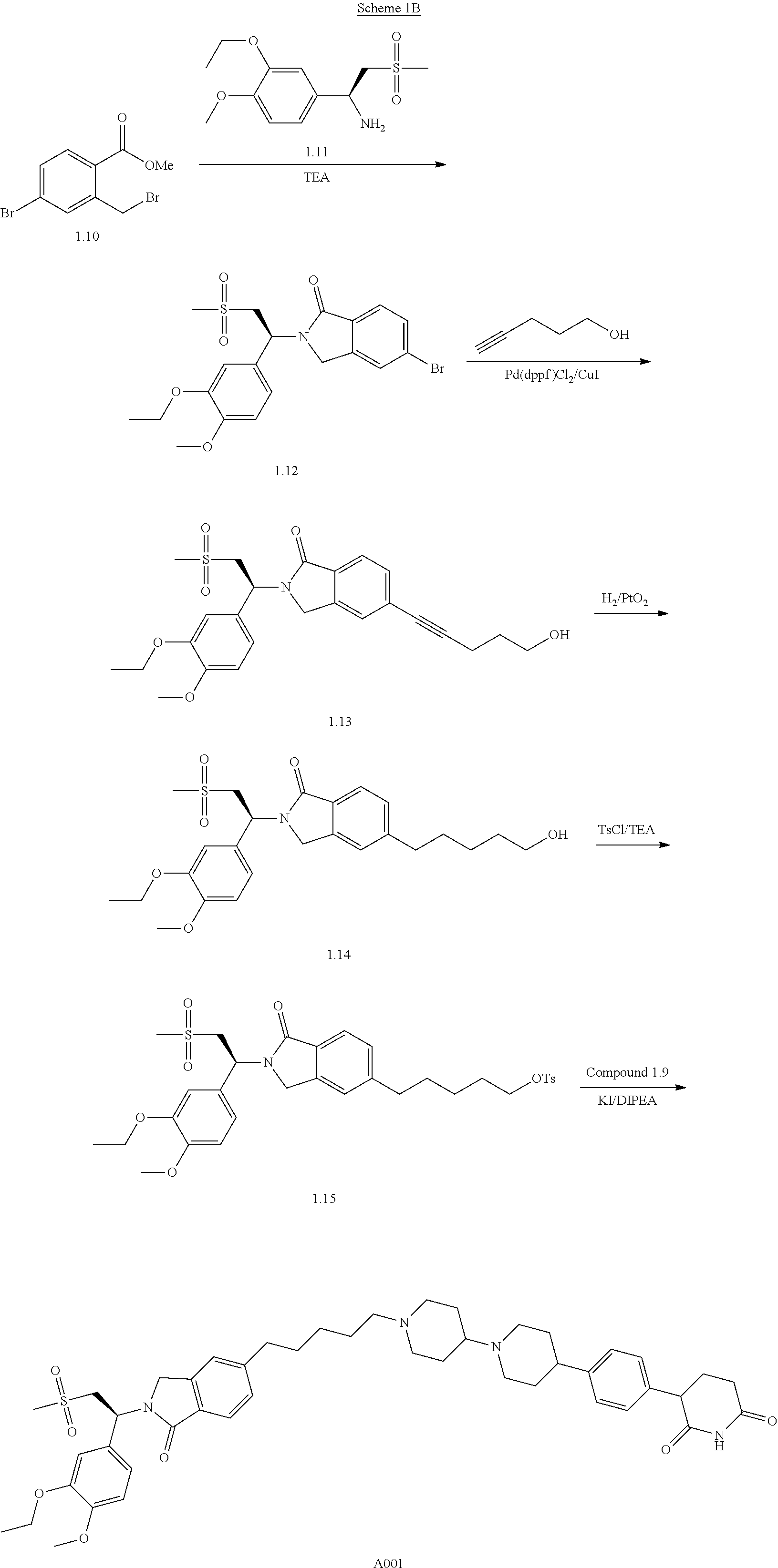

Preparation of tert-butyl 4-(4-(2,6-dioxopiperidin-3-yl)phenyl)piperidine-1-carboxylate 1.6. To a solution of compound 1.5 (78 g, 142 mmol) in EtOH (700 mL) under N₂ was added 10% Pd/C (40 g). After hydrogenated at 100 psi of H₂ for 16 h, the reaction mixture was filtered through celite and the celite was washed with EtOH (2×350 mL). The combined organic phase was concentrated under vacuum to yield a crude product, which was purified by silica-gel column chromatography eluting with EtOAc/heptane to afford compound 1.6 (25 g) in 47% yield.

Preparation of 3-(4-(piperidin-4-yl) phenyl)piperidine-2,6-dione hydrochloride 1.7. To a solution of compound 1.6 in dioxane (15 mL) at 0° C. was added 4M HCl in dioxane (70 mL). After stirred for 2 h, the reaction mixture was concentrated under vacuum to afford a crude product, which was triturated with IPA/ACN (20 mL, 1:1) to afford compound 1.7 (10.3 g) in 86% yield. ¹H NMR (400 MHz, DMSO-d₆) δ 10.81 (s, 1H), 9.18-8.99 (m, 1H), 7.19 (s, 4H), 3.83 (dd, J=4.8, 11.4 Hz, 1H), 3.59-3.54 (m, 2H), 3.33 (d, J=12.3 Hz, 2H), 3.03-2.91 (m, 2H), 2.89-2.78 (m, 1H), 2.69-2.64 (m, 1H), 2.20-2.16 (m, 1H), 2.07-1.98 (m, 1H), 1.95-1.84 (m, 4H).

Preparation of tert-butyl 4-(4-(2,6-dioxopiperidin-3-yl)phenyl)-[1,4′-bipiperidine]-1′-carboxylate 1.8. To a solution of compound 1.7 (5 g, 18.38 mmol) and tert-butyl 4-oxopiperidine-1-carboxylate (5.4 g, 27.6 mmol) in EtOH (50 mL) and DCM (50 mL) at 0° C. was added NaOAc (15 g, 184 mmol). The mixture was stirred for 3 h, followed by addition of NaBH₃CN (3.4 g, 55.1 mmol) at 0° C. After stirred for 16 h, the reaction mixture was diluted with water (250 mL) and extracted with 5% MeOH in DCM (2×300 mL). The combined organic layers were dried over anhydrate Na₂SO₄ and concentrated under vacuum to yield a crude product, which was purified by silica-gel column chromatography eluting with MeOH/DCM to afford compound 1.8 (3.5 g) in 42% yield. MS (ESI) mz: 456.5 [M+H]⁺.

Preparation of 3-(4-([1,4′-bipiperidin]-4-yl) phenyl) piperidine-2,6-dione hydrochloride 1.9. To a solution of compound 1.8 (3.5 g, 7.69 mmol) in dioxane (35 mL) at 0° C. was added 4M HCl in dioxane (17.5 mL). After stirred for 16 h, the reaction mixture was concentrated under vacuum to afford compound 1.9 (2.5 g) in 93% yield, which was used directly in the next step without further purification. MS (ESI) m/i: 356.4 [M+H]⁺.

Preparation of (S)-5-bromo-2-(1-(3-ethoxy-4-methoxyphenyl)-2-(methyl-sulfonyl)ethyl)isoindolin-1-one 1.12. A mixture of methyl 4-bromo-2-(bromomethyl)benzoate 1.10 (3 g, 9.74 mmol), (S)-1-(3-ethoxy-4-methoxyphenyl)-2-(methylsulfonyl)ethan-1-amine 1.11 (2.66 g, 9.74 mmol), and TEA (1.97 g, 19.5 mmol) in ACN (30 mL) was stirred at 90° C. for 12 h, followed by addition of K₂CO₃ (1.35 g, 9.74 mmol). After stirred at 90° C. for 12 h, the reaction mixture was diluted with water (100 mL) and extracted with EtOAc (30 mL×3). The combined organic layers were concentrated under reduced pressure to yield a crude product, which was purified by silica-gel column chromatography eluting with EtOAc/PE to afford compound 1.12 (2.2 g) in 47% yield. ¹H NMR (400 MHz, DMSO-d₆) δ 7.83 (s, 1H), 7.72-7.57 (m, 2H), 7.01 (s, 1H), 6.94 (s, 2H), 5.88 (dd, J=4.0, 10.4 Hz, 1H), 4.61 (d, J=18.0 Hz, 1H), 4.28 (dd, J=10.4, 14.4 Hz, 1H), 4.15-4.04 (m, 2H), 4.00-3.86 (m, 2H), 3.73 (s, 3H), 3.00 (s, 3H), 1.31 (t, J=6.8 Hz, 3H); MS (ESI) m/z: 468.1 [M+H]⁺.

Preparation of(S)-2-(1-(3-ethoxy-4-methoxyphenyl)-2-(methylsulfonyl)ethyl)-5-(5-hydroxypent-1-yn-1-yl)isoindolin-1-one 1.13. A mixture of compound 1.12 (500 mg, 1.07 mmol), pent-4-yn-1-ol (135 mg, 1.60 mmol), TEA (1.08 g, 10.7 mmol), CuI (20.3 mg, 107 μmol), and Pd(dppf)Cl₂ (78 mg, 107 μmol) in DMF (5 mL) was stirred at 80° C. for 3 h under N₂. The reaction mixture was then diluted with water (100 mL) and extracted with EtOAc (20 mL×3). The combined organic layers were concentrated under reduced pressure to yield a crude product, which was purified by silica-gel column chromatography eluting with EtOAc/PE to afford compound 1.13 (270 mg) in 52% yield. ¹H NMR (400 MHz, DMSO-d₆) δ 7.64 (d, J=8.0 Hz, 1H), 7.56 (s, 1H), 7.46 (d, J=7.6 Hz, 1H), 7.01 (s, 1H), 6.94 (s, 2H), 5.88 (dd, J=4.0, 10.4 Hz, 1H), 4.65-4.49 (m, 2H), 4.27 (dd, J=10.4, 14.4 Hz, 1H), 4.12-4.02 (m, 2H), 4.01-3.88 (m, 2H), 3.73 (s, 3H), 3.51 (q, J=6.4 Hz, 2H), 2.99 (s, 3H), 2.49-2.45 (m, 2H), 1.68 (q, J=6.4 Hz, 2H), 1.31 (t, J=6.8 Hz, 3H); MS (ESI) m/z: 472.4 [M+H]⁺.

Preparation of(S)-2-(1-(3-ethoxy-4-methoxyphenyl)-2-(methylsulfonyl)ethyl)-5-(5-hydroxypentyl)isoindolin-1-one 1.14. A mixture of compound 1.13 (220 mg, 467 μmol) and PtO₂ (10.6 mg, 46.7 μmol) in THF (3 mL) was stirred at 20° C. for 3 h under H₂. The reaction mixture was filtered and concentrated under reduced pressure to afford compound 1.14 (295 mg), which was used directly in the next step without further purification. ¹H NMR (400 MHz, DMSO-d₆) δ 7.58 (d, J=8.0 Hz, 1H), 7.36 (s, 1H), 7.29 (d, J=8.0 Hz, 1H), 7.01 (s, 1H), 6.98-6.89 (m, 2H), 5.88 (dd, J=4.0, 10.4 Hz, 1H), 4.57 (d, J=17.2 Hz, 1H), 4.32 (t, J=5.2 Hz, 1H), 4.26 (dd, J=10.4, 14.4 Hz, 1H), 4.10-3.97 (m, 3H), 3.92 (dd, J=4.4, 14.4 Hz, 1H), 3.73 (s, 3H), 3.60 (s, 2H), 2.99 (s, 3H), 2.65 (t, J=7.6 Hz, 2H), 1.76 (td, J=3.2, 6.4 Hz, 3H), 1.57 (q, J=7.6 Hz, 2H), 1.48-1.37 (m, 2H), 1.37-1.31 (m, 2H); MS (ESI) m/z: 476.3 [M+H]⁻.

Preparation of(S)-5-(2-(1-(3-ethoxy-4-methoxyphenyl)-2-(methylsulfonyl)ethyl)-1-oxoisoindolin-5-yl)pentyl 4-methylbenzenesulfonate 1.15. To a solution of compound 1.14 (240 mg, 505 μmol) and TEA (153 mg, 1.51 mmol) in DCM (4.8 mL) was added TsCl (481 mg, 2.52 mmol) at 0° C. After stirred at 20° C. for 12 h, the reaction mixture was diluted with water (20 mL) and extracted with DCM (10 mL×3). The combined organic layers were concentrated under reduced pressure to yield a crude product, which was purified by silica-gel column chromatography eluting with EtOAc/PE to afford compound 1.15 (215 mg) in 64% yield. ¹H NMR (400 MHz, DMSO-d₆) δ 7.75 (d, J=8.4 Hz, 2H), 7.57 (d, J=7.8 Hz, 1H), 7.45 (d, J=8.0 Hz, 2H), 7.32 (s, 1H), 7.25 (d, J=7.8 Hz, 1H), 7.01 (s, 1H), 6.97-6.87 (m, 2H), 5.89 (dd, J=4.0, 10.4 Hz, 1H), 4.33-4.20 (m, 1H), 4.12-4.04 (m, 2H), 4.04-3.96 (m, 4H), 3.93 (dd, J=4.4, 14.8 Hz, 1H), 3.73 (s, 3H), 2.99 (s, 3H), 2.58 (t, J=7.6 Hz, 2H), 2.39 (s, 3H), 1.63-1.52 (m, 2H), 1.47 (q, J=7.6 Hz, 2H), 1.31 (t, J=7.2 Hz, 3H), 1.27-1.17 (m, 2H); MS (ESI) m/z: 630.3 [M+H]⁺.

Preparation of 3-(4-(1′-(5-(2-((S)-1-(3-ethoxy-4-methoxyphenyl)-2-(methyl-sulfonyl)ethyl)-1-oxoisoindolin-5-yl)pentyl)-[1,4′-bipiperidin]-4-yl)phenyl)piperidine-2,6-dione A001. A mixture of 3-(4-([1,4′-bipiperidin]-4-yl)phenyl)piperidine-2,6-dione 1.9 (100 mg, 255 μmol), DIPEA (165 mg, 1.28 mmol), and K₂CO₃ (35.3 mg, 255 μmol) in DMF (3 mL) was stirred at 20° C. for 30 min, followed by addition of compound 1.15 (160.7 mg, 255 μmol) and KI (85 mg, 510 μmol). After stirred at 80° C. for 5 h, the reaction mixture was diluted ACN (1 mL) and adjusted to pH 5-6 with TFA at 0° C. The reaction mixture was concentrated under reduced pressure to yield a crude product, which was purified by reverse-phase prep-HPLC to afford compound A001. ¹H NMR (400 MHz, DMSO-d₆) δ 10.82 (s, 1H), 8.25-8.17 (m, 1H), 7.58 (d, J=7.6 Hz, 1H), 7.37 (s, 1H), 7.30 (d, J=8.0 Hz, 1H), 7.25-7.16 (m, 2H), 7.15-7.07 (m, 2H), 7.01 (s, 1H), 6.93 (s, 2H), 5.89 (dd, J=4.0, 10.4 Hz, 1H), 4.57 (d, J=17.2 Hz, 1H), 4.26 (dd, J=10.4, 14.4 Hz, 1H), 4.10-3.98 (m, 3H), 3.92 (dd, J=4.0, 14.4 Hz, 1H), 3.80 (dd, J=5.2, 11.6 Hz, 1H), 3.72 (s, 3H), 2.99 (s, 3H), 2.98-2.86 (m, 3H), 2.72-2.58 (m, 3H), 2.45 (s, 2H), 2.34-2.24 (m, 5H), 2.24-2.06 (m, 2H), 2.05-1.98 (m, 1H), 1.95-1.90 (m, 2H), 1.74 (s, 4H), 1.68-1.53 (m, 4H), 1.52-1.36 (m, 4H), 1.28 (s, 5H); MS (ESI) m/z: 813.3 [M+H]⁺.

### Example 2

### Preparation of N-(6-(5-(4-(4-(2,6-dioxopiperidin-3-yl)phenyl)-[1,4′-bipiperidin]-1′-yl)pentyl)-2-((S)-1-(3-ethoxy-4-methoxyphenyl)-2-(methylsulfonyl)ethyl)-1,3-dioxoisoindolin-4-yl)-acetamide A004

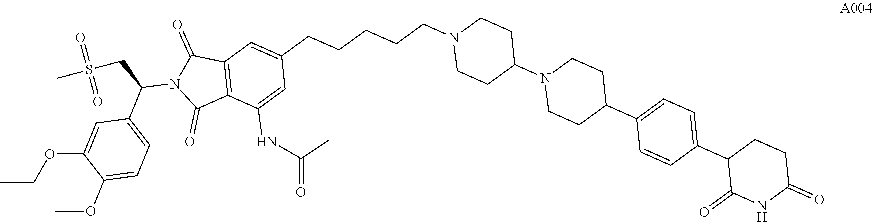 [SMILES: CCOc1cc([C@@H](CS(C)(=O)=O)N2C(=O)c3cc(CCCCCN4CCC(N5CCC(c6ccc(C7CCC(=O)NC7=O)cc6)CC5)CC4)cc(NC(C)=O)c3C2=O)ccc1OC]

Compound A004 was prepared as shown in Scheme 2.

Preparation of 5-bromo-3-nitrophthalic acid 2.1. To a solution of 5-bromo-2-methyl-3-nitrobenzoic acid (20 g, 76.91 mmol) in H₂O (200 mL) was added NaOH (9.23 g, 231 mmol) at 0° C., followed by addition of KMnO₄ (38.7 g, 245 mmol) portionwise at 80° C. After stirred at 80° C. for 12 h, the reaction mixture was filtered, and the cake was washed with hot water (200 mL×3). The filtrate was adjusted with 2N HCl to pH 1 and extracted with EtOAc (200 mL×3). The combined organic layers were dried over anhydrous Na₂SO₄, filtered, and concentrated under reduced pressure to yield a crude product, which was purified by silica-gel column chromatography eluting with MeOH/DCM to yield compound 2.1 (11 g) in 49% yield. ¹H NMR (400 MHz, DMSO-d₆) δ 14.42-13.78 (m, 2H), 8.58 (d, J=2.0 Hz, 1H), 8.39 (d, J=2.0 Hz, 11H).

Preparation of(S)-6-bromo-2-(1-(3-ethoxy-4-methoxyphenyl)-2-(methyl-sulfonyl)ethyl)-4-nitroisoindoline-1,3-dione 2.2. A mixture of (1S)-1-(3-ethoxy-4-methoxy-phenyl)-2-methylsulfonylethanamine 1.11 (2.07 g, 7.59 mmol) and compound 2.1 (2 g, 6.9 mmol) in AcOH (10 mL) was stirred at 100° C. for 12 h. The reaction mixture was then concentrated under reduced pressure to yield a crude product, which was purified by silica-gel column chromatography eluting with EtOAc/PE to afford compound 2.2 in 61% yield. ¹H NMR (400 MHz, DMSO-d₆) δ 8.62 (d, J=1.6 Hz, 11H), 8.46 (d, J=1.6 Hz, 1H), 7.09 (d, J=2.0 Hz, 1H), 7.03-6.98 (m, 1H), 6.96-6.92 (m, 1H), 5.78 (dd, J=5.6, 8.8 Hz, 1H), 4.24-4.21 (m, 1H), 4.06-4.00 (m, 4H), 3.74 (s, 3H), 2.98 (s, 3H), 1.32 (t, J=7.2 Hz, 3H); MS (ESI) m/z: 549.1 [M+Na]⁺.

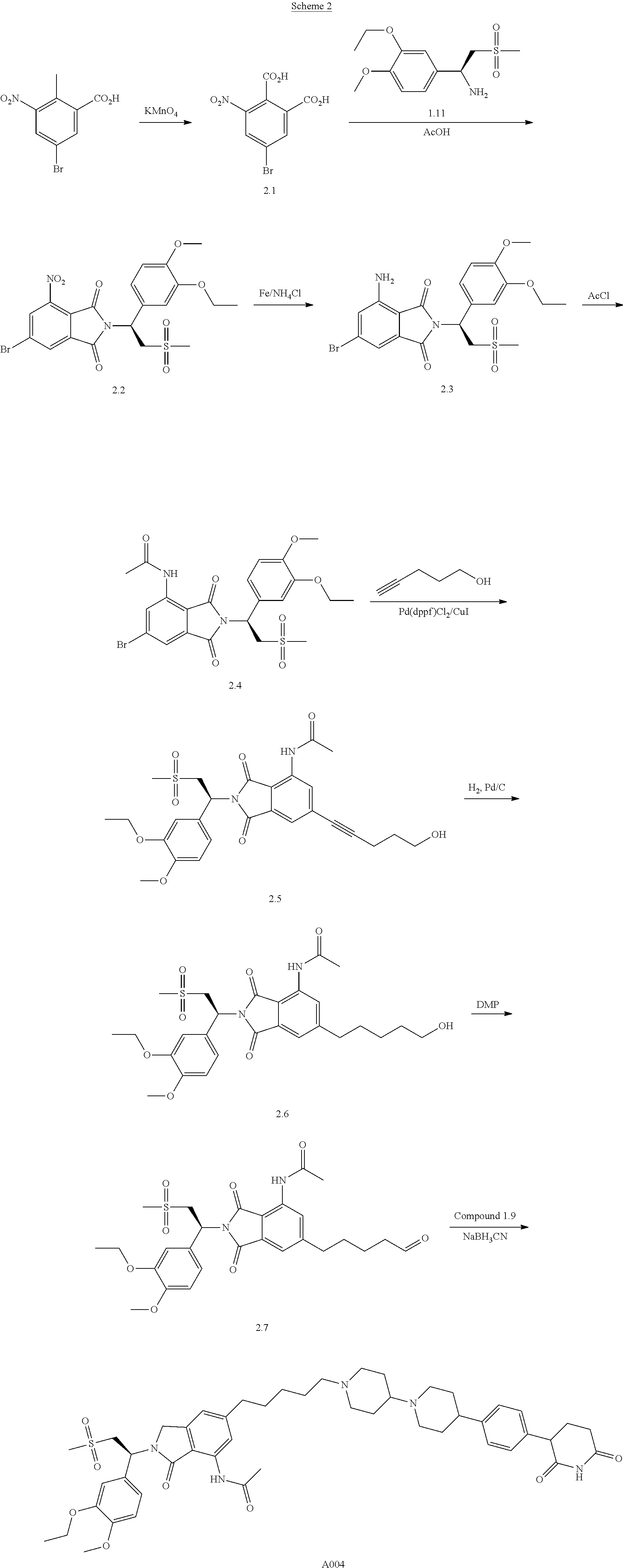

Preparation of (S) 4-amino-6-bromo-2-(1-(3-ethoxy-4-methoxyphenyl)-2-(methylsulfonyl)ethyl)isoindoline-1,3-dione 2.3. To a mixture of compound 2.2 (2 g, 3.79 mmol) in EtOH (10 mL) and H₂O (10 mL) were added Fe (1.06 g, 19 mmol) and NH₄Cl (2.03 g, 37.9 mmol) at 20° C. After stirred at 80° C. for 8 h, the reaction mixture was filtered and concentrated under reduced pressure to yield a crude product, which was purified by silica-gel column chromatography eluting with EtOAc/PE to afford compound 2.3 (1.8 g) in 95% yield. ¹H NMR (400 MHz, DMSO-d₆) δ 7.22-7.18 (m, 1H), 7.09-7.03 (m, 2H), 6.93 (s, 2H), 6.69 (s, 2H), 5.70 (dd, J=4.0, 10.0 Hz, 1H), 4.31 (dd, J=10.8, 14.4 Hz, 1H), 4.12-4.05 (m, 1H), 4.02-3.98 (m, 2H), 3.73 (s, 3H), 3.00 (s, 3H), 1.32 (t, J=6.8 Hz, 3H).

Preparation of (S)—N-(6-bromo-2-(1-(3-ethoxy-4-methoxyphenyl)-2-(methyl-sulfonyl)ethyl)-1,3-dioxoisoindolin-4-yl)acetamide 2.4. To a mixture of compound 2.3 (1.2 g, 2.41 mmol) in THF (10 mL) were added acetyl chloride (284 mg, 3.62 mmol), DMF (17.6 mg, 241 μmol), and TEA (488 mg, 4.83 mmol) successively. After stirred at 20° C. for 12 h, the reaction mixture was diluted with H₂O (10 mL) and extracted with EtOAc (20 mL×3). The combined organic layers were dried over anhydrous Na₂SO₄, filtered, and concentrated under reduced pressure to yield a crude product, which was purified by silica-gel column chromatography eluting with EtOAc/PE to afford compound 2.4 (1.1 g) in 85% yield. ¹H NMR (400 MHz, DMSO-d₆) δ 9.75 (s, 1H), 8.66 (s, 1H), 7.80-7.75 (m, 1H), 7.06 (s, 1H), 7.01-6.88 (m, 2H), 5.78 (d, J=4.4 Hz, 1H), 4.30 (dd, J=10.4, 14.4 Hz, 1H), 4.21-4.11 (m, 1H), 4.06-4.00 (m, 2H), 3.74 (s, 3H), 3.01 (s, 3H), 2.20 (s, 3H), 1.32 (t, J=6.8 Hz, 3H).

Preparation of (S)—N-(2-(1-(3-ethoxy-4-methoxyphenyl)-2-(methylsulfonyl)ethyl)-6-(5-hydroxypent-1-yn-1-yl)-1,3-dioxoisoindolin-4-yl)acetamide 2.5. To a solution of compound 2.4 (500 mg, 927 μmol) and pent-4-yn-1-ol (234 mg, 2.78 mmol) in DMF (10 mL) were added TEA (469 mg, 4.63 mmol), Pd(PPh₃)₂Cl₂ (97.6 mg, 139 μmol), and CuI (17.7 mg, 92.7 μmol) at 20° C. After stirred at 80° C. for 12 h, the reaction mixture was diluted with H₂O (50 mL) at 20° C. and extracted with EtOAc (50 mL×3). The combined organic layers were dried over anhydrous Na₂SO₄, filtered, and concentrated under reduced pressure to yield a crude product, which was purified by silica-gel column chromatography eluting with EtOAc/PE to afford compound 2.5 (800 mg) in 80% yield. MS (ESI) m/z: 543.2 [M+H]⁺.

Preparation of (S)—N-(2-(1-(3-ethoxy-4-methoxyphenyl)-2-(methylsulfonyl)ethyl)-6-(5-hydroxypentyl)-1,3-dioxoisoindolin-4-yl)acetamide 2.6. To a mixture of compound 2.5 (780 mg, 1.44 mmol) in THF (10 mL) and EtOH (10 mL) was add PtO₂ (300 mg, 1.32 mmol) under N₂. After stirred under H₂ (15 psi) at 30° C. for 12 h, the reaction mixture was filtered and concentrated under reduced pressure to afford compound 2.6 (700 mg), which was used directly for the next step without further purification. ¹H NMR (400 MHz, DMSO-d₆) δ 9.64 (s, 1H), 8.28 (s, 1H), 7.43 (s, 1H), 7.07 (s, 1H), 7.00-6.90 (m, 2H), 5.76 (dd, J=4.0, 10.4 Hz, 1H), 4.39-4.27 (m, 2H), 4.13 (dd, J=4.0, 14.4 Hz, 1H), 4.02 (q, J=6.8 Hz, 2H), 3.74 (s, 3H), 3.37 (q, J=6.4 Hz, 3H), 3.02 (s, 3H), 2.71 (t, J=7.6 Hz, 2H), 2.18 (s, 3H), 1.64-1.55 (m, 2H), 1.44-1.37 (m, 2H), 1.35-1.29 (m, 6H).

Preparation of (S)—N-(2-(1-(3-ethoxy-4-methoxyphenyl)-2-(methylsulfonyl)ethyl)-1,3-dioxo-6-(5-oxopentyl)isoindolin-4-yl)acetamide 2.7. To a solution of compound 2.6 (300 mg, 549 μmol) in DCM (10 mL) was added DMP (233 mg, 549 μmol) portionwise at 0° C. After stirred at 20° C. for 2 h, the reaction mixture was diluted with H₂O (10 mL) and extracted with DCM (10 mL×3). The combined organic layers were dried over anhydrous Na₂SO₄, filtered, and concentrated under reduced pressure to yield a crude product, which was purified by silica-gel column chromatography eluting with EtOAc/PE to afford compound 2.7 (242 mg) in 81% yield. MS (ESI) m/z: 545.2 [M+H]⁺.

Preparation of N-(6-(5-(4-(4-(2,6-Dioxopiperidin-3-yl)phenyl)-[1,4′-bipiperidin]-1′-yl)pentyl)-2-((S)-1-(3-ethoxy-4-methoxyphenyl)-2-(methylsulfonyl)ethyl)-1,3-dioxo-isoindolin-4-yl)-acetamide A004. To a mixture of compound 2.7 (230 mg, 422 μmol) and 3-[4-[1-(4-piperidyl)-4-piperidyl]phenyl]piperidine-2,6-dione (100 mg, 281 μmol) in MeOH (5 mL) and DCM (5 mL) were added AcOH (16.9 mg, 281 μmol), NaOAc (23.1 mg, 281 μmol), and NaBH₃CN (35.4 mg, 563 μmol) successively. After stirred at 20° C. for 12 h, the reaction mixture was diluted with H₂O (10 mL) and extracted with DCM (10 mL×3). The combined organic layers were dried over anhydrous Na₂SO₄, filtered, and concentrated under reduced pressure to yield a crude product, which was purified by reverse-phase prep-HPLC to afford compound A004 (38.5 mg) in 16% yield. ¹H NMR (400 MHz, DMSO-d₆) δ 10.80 (s, 1H), 9.64 (s, 1H), 8.28 (s, 1H), 8.20 (s, 1H), 7.43 (s, 1H), 7.25-7.16 (m, 2H), 7.16-7.09 (m, 2H), 7.06 (s, 1H), 7.00-6.90 (m, 2H), 5.76 (dd, J=3.6, 10.0 Hz, 1H), 4.39-4.28 (m, 1H), 4.12 (dd, J=4.0, 14.4 Hz, 1H), 4.01 (q, J=6.8 Hz, 2H), 3.80 (dd, J=4.4, 10.8 Hz, 1H), 3.73 (s, 3H), 3.01 (s, 3H), 2.99-2.88 (m, 4H), 2.71 (t, J=7.2 Hz, 2H), 2.67-2.59 (m, 1H), 2.45 (s, 2H), 2.35-2.20 (m, 6H), 2.18 (s, 3H), 2.07-1.98 (m, 1H), 1.94-1.83 (m, 2H), 1.78-1.67 (m, 4H), 1.65-1.54 (m, 4H), 1.50-1.38 (m, 4H), 1.36-1.25 (m, 5H); MS (ESI) m/z: 884.4 [M+H]⁺.

### Example 3

### Preparation of 4-(4-(2-(4-(4-(2,6-dioxopiperidin-3-yl)phenyl)-[1,4′-bipiperidin]-1′-yl)ethyl)-piperidin-1-yl)-2-((S)-1-(3-ethoxy-4-methoxyphenyl)-2-(methylsulfonyl)ethyl)isoindoline-1,3-dione A031

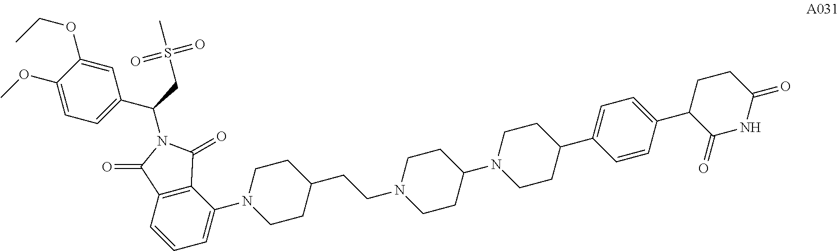 [SMILES: CCOc1cc([C@@H](CS(C)(=O)=O)N2C(=O)c3cccc(N4CCC(CCN5CCC(N6CCC(c7ccc(C8CCC(=O)NC8=O)cc7)CC6)CC5)CC4)c3C2=O)ccc1OC]

Compound A031 was prepared as shown in Scheme 3.

Preparation of(S)-2-(1-(3-ethoxy-4-methoxyphenyl)-2-(methylsulfonyl)ethyl)-4-(4-(2-hydroxyethyl)piperidin-1-yl)isoindoline-1,3-dione 3.2. To a solution of (S)-2-(1-(3-ethoxy-4-methoxyphenyl)-2-(methylsulfonyl)ethyl)-4-fluoroisoindoline-1,3-dione 3.1 (450 mg, 1.07 mmol) and 2-(piperidin-4-yl)ethan-1-ol (166 mg, 1.28 mmol) in DMSO (5 mL) was added DIPEA (414 mg, 3.20 mmol). After stirred at 100° C. under N₂ for 2 h, the reaction mixture was diluted with water (5 mL) and extracted with EtOAc (10 mL×4). The combined organic layers were washed with brine (50 mL×2), dried over anhydrous Na₂SO₄, filtered, and concentrated under reduced pressure to yield a crude product, which was purified by silica-gel column chromatography eluting with EtOAc/PE to afford compound 3.2 (510 mg) in 90% yield. ¹H NMR (400 MHz, DMSO-d₆) δ 7.63 (t, J=8.0 Hz, 1H), 7.28 (t, J=8.0 Hz, 2H), 7.08 (s, 1H), 6.93 (s, 2H), 5.76 (dd, J=4.4, 10.0 Hz, 1H), 4.40-4.37 (m, 1H), 4.37-4.31 (m, 1H), 4.09 (dd, J=4.4, 14.4 Hz, 1H), 4.01 (q, J=7.2 Hz, 2H), 3.73 (s, 3H), 3.66 (br t, J=13.6 Hz, 2H), 3.48 (q, J=6.4 Hz, 2H), 2.99 (s, 3H), 2.87-2.76 (m, 2H), 2.54 (s, 3H), 1.75 (br d, J=11.2 Hz, 2H), 1.43 (q, J=6.8 Hz, 2H), 1.31 (t, J=6.8 Hz, 3H); MS (ESI) mz: 531.2 [M+H]⁺.

Preparation of(S)-2-(1-(2-(1-(3-ethoxy-4-methoxyphenyl)-2-(methylsulfonyl)-ethyl)-1,3-dioxoisoindolin-4-yl)piperidin-4-yl)ethyl 4-methylbenzenesulfonate 3.3. To a solution of compound 3.2 (310 mg, 584 μmol) in DCM (3 mL) at 0° C. were added TsCl (133 mg, 701 μmol) and TEA (177 mg, 1.75 mmol). After stirred under N₂ for 12 h, the reaction mixture was diluted water (4 mL) and extracted with DCM (5 mL×2). The combined organic layers were washed with brine (10 mL×2), dried over Na₂SO₄, filtered, and concentrated under reduced pressure to yield a crude product, which was purified by prep-TLC (EtOAc/PE) to afford compound 3.3 (200 mg) in 50% yield. MS (ESI) m/z: 685.0 [M+H]⁺.

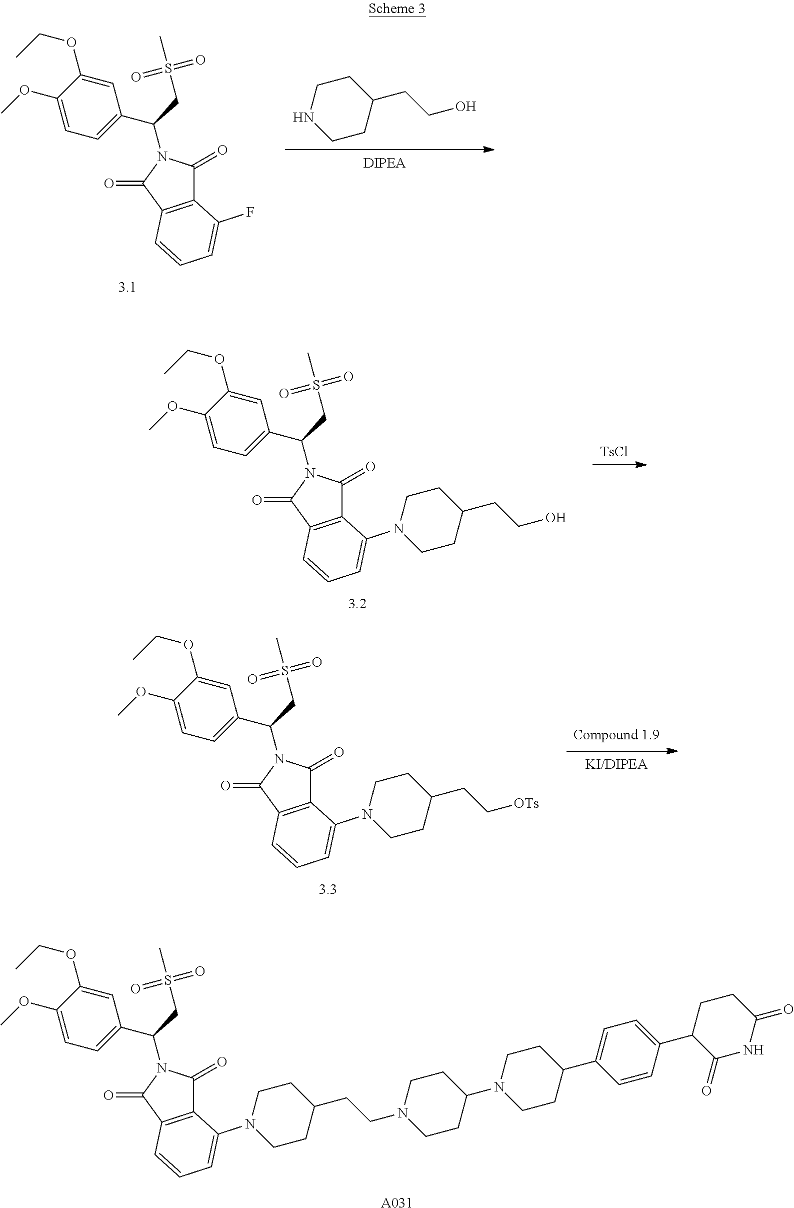

Preparation of 4-(4-(2-(4-(4-(2,6-dioxopiperidin-3-yl)phenyl)-[1,4′-bipiperidin]-1′-yl)ethyl)-piperidin-1-yl)-2-((S)-1-(3-ethoxy-4-methoxyphenyl)-2-(methylsulfonyl)ethyl)-isoindoline-1,3-dione A031. To a solution of compound 1.9 (108 mg, 275 μmol, HCl) and compound 3.3 (188 mg, 275 μmol) in DMF (2 mL) were added DIPEA (106 mg, 826 μmol) and KI (9.15 mg, 55 μmol). After stirred at 80° C. under N₂ for 4 h, the reaction mixture was filtered and purified by reverse-phase prep-HPLC to afford compound A031 (20.6 mg) in 8.4% yield. ¹H NMR (400 MHz, DMSO-d₆) δ 10.81 (s, 1H), 8.25 (s, 1H), 7.66-7.61 (m, 1H), 7.31-7.25 (m, 2H), 7.21-7.17 (m, 2H), 7.15-7.11 (m, 2H), 7.08 (s, 1H), 6.93 (s, 2H), 5.76 (dd, J=4.0, 10.4 Hz, 1H), 4.35 (dd, J=10.4, 14.4 Hz, 1H), 4.09 (dd, J=4.4, 14.4 Hz, 1H), 4.01 (q, J=7.2 Hz, 2H), 3.80 (dd, J=4.8, 11.2 Hz, 1H), 3.72 (s, 3H), 3.69-3.57 (m, 5H), 3.01 (br s, 3H), 2.99 (s, 3H), 2.85-2.77 (m, 2H), 2.70-2.61 (m, 1H), 2.46 (br d, J=4.0 Hz, 1H), 2.40 (br d, J=5.2 Hz, 2H), 2.37-2.28 (m, 3H), 2.21-2.11 (m, 1H), 2.06-1.95 (m, 3H), 1.81-1.73 (m, 6H), 1.69-1.60 (m, 2H), 1.57-1.49 (m, 2H), 1.44 (br d, J=5.6 Hz, 3H), 1.39-1.35 (m, 1H), 1.31 (t, J=7.2 Hz, 3H); MS (ESI) m/z: 868.2 [M+H]⁺.

### Example 4

### Preparation of 4-((1-(2-(4-(4-(2,6-dioxopiperidin-3-yl)phenyl)piperidin-1-yl)ethyl)piperidin-4-yl)amino)-2-((S)-1-(3-ethoxy-4-methoxyphenyl)-2-(methylsulfonyl)ethyl)isoindoline-1,3-dione A036

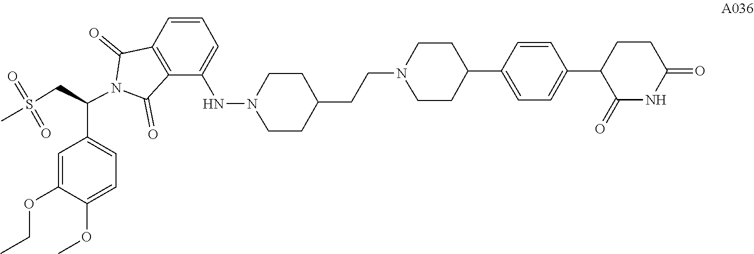 [SMILES: CCOc1cc([C@@H](CS(C)(=O)=O)N2C(=O)c3cccc(NN4CCC(CCN5CCC(c6ccc(C7CCC(=O)NC7=O)cc6)CC5)CC4)c3C2=O)ccc1OC]

Compound A036 was prepared as shown in Scheme 4.

Preparation of 3-[4-[1-(2-hydroxyethyl)-4-piperidyl]phenyl]piperidine-2,6-dione 4.1. A mixture of 3-[4-(4-piperidyl)phenyl]piperidine-2,6-dione 1.7 (300 mg, 1.1 mmol), 2-iodoethanol (379 mg, 2.2 mmol), K₂CO₃ (304 mg, 2.2 mmol), and KI (183 mg, 1.1 mmol) in methoxycyclopentane (2 mL) was stirred at 80° C. under N₂ for 12 h. The reaction mixture was then concentrated under reduced pressure to afford compound 4.1 (300 mg) in 86% yield, which was used directly in the next step without further purification. MS (ESI) m/z: 317.1 [M+H]⁺.

Preparation of 3-[4-[1-(2-bromoethyl)-4-piperidyl]phenyl]piperidine-2,6-dione 4.2. To a solution of compound 4.1 (200 mg, 632 μmol) in ACN (2 mL) was added PBr₃ (342 mg, 1.26 mmol). After stirred at 80° C. for 2 h, the reaction mixture was diluted with EtOAc (5 mL). The resulting precipitates were collected by filtration and dried under reduced pressure to afford compound 4.2 (80 mg) in 33% yield, which was used directly in the next step without further purification. MS (ESI) m/z: 379.1 [M+H]⁺.

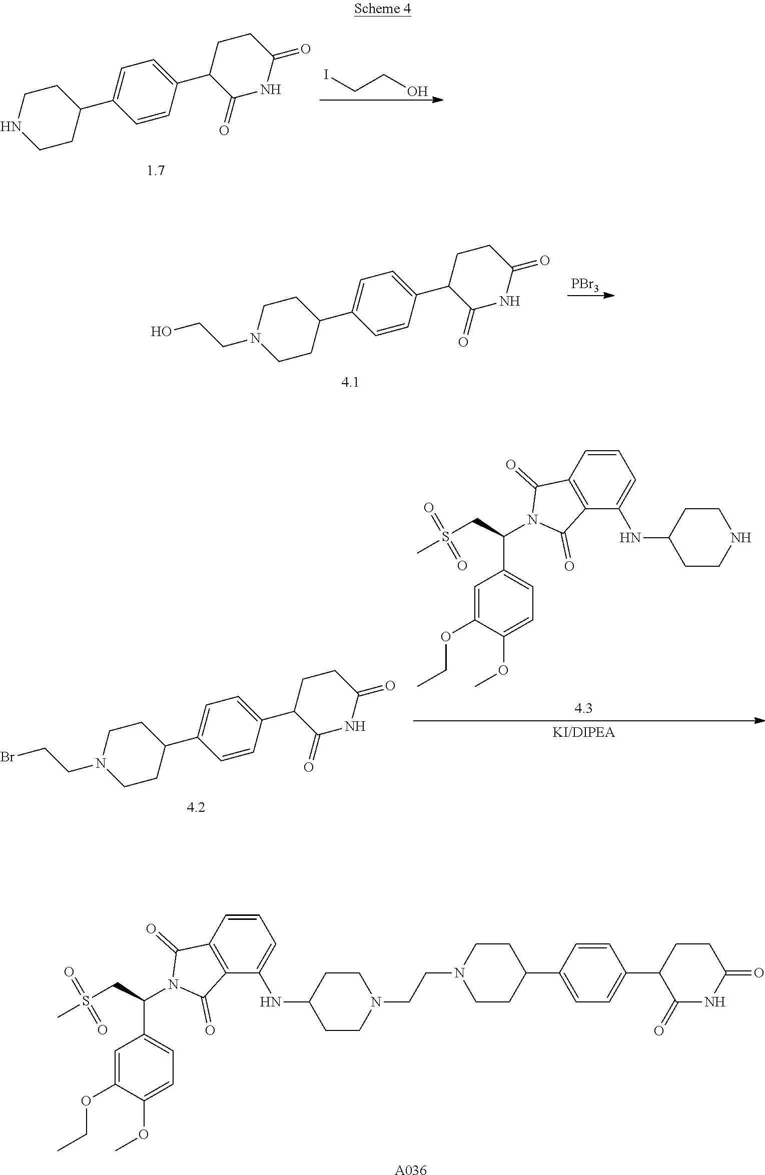

Preparation of 4-((1-(2-(4-(4-(2,6-Dioxopiperidin-3-yl)phenyl)piperidin-1-yl)-ethyl)piperidin-4-yl)amino)-2-((S)-1-(3-ethoxy-4-methoxyphenyl)-2-(methylsulfonyl)ethyl)-isoindoline-1,3-dione A036. To a solution of compound 4.2 (80 mg, 211 μmol) and 2-[(1R)-1-(3-ethoxy-4-methoxy-phenyl)-2-methylsulfonyl-ethyl]-4-(4-piperidyl-amino)isoindoline-1,3-dione 4.3 (106 mg, 211 μmol) in DMF (1 mL) were added DIPEA (82 mg, 633 μmol) and KI (1.75 mg, 11 μmol). After stirred at 80° C. for 3 h, the reaction mixture was diluted with H₂O (5 mL) and extracted with EtOAc (15 mL). The combined organic layers were washed with brine (20 mL), dried over anhydrous Na₂SO₄, filtered, and concentrated under reduced pressure to yield a crude product, which was purified by silica-gel column chromatography eluting with MeOH/DCM and further purified by reverse-phase prep-HPLC to afford compound A036 (6.66 mg) in 3% yield. ¹H NMR (400 MHz, CDCl₃) δ 7.94 (s, 1H), 7.49-7.37 (m, 1H), 7.25 (s, 1H), 7.17-7.11 (m, 3H), 7.04 (d, J=6.8 Hz, 1H), 6.88-6.78 (m, 2H), 6.32-6.22 (m, 1H), 5.83 (dd, J=4.8, 9.6 Hz, 1H), 4.51 (d, J=14.4 Hz, 1H), 4.17-4.06 (m, 2H), 3.85 (s, 3H), 3.82-3.72 (m, 2H), 3.54-3.40 (m, 1H), 3.23-3.11 (m, 1H), 3.01-2.86 (m, 2H), 2.81 (s, 3H), 2.74-2.64 (m, 4H), 2.59-2.50 (m, 1H), 2.32-2.23, (m, 4H), 2.10-2.02 (m, 2H), 1.92-1.81 (m, 3H), 1.64 (s, 8H), 1.47 (t, J=7.2 Hz, 3H); MS (ESI) m/z: 800.5 [M+H]⁺.

### Example 5

### Preparation of 4-((1-((1-(4-(2,6-dioxopiperidin-3-yl)benzyl)piperidin-4-yl)methyl)piperidin-4-yl)amino)-2-((S)-1-(3-ethoxy-4-methoxyphenyl)-2-(methylsulfonyl)ethyl)isoindoline-1,3-dione A037

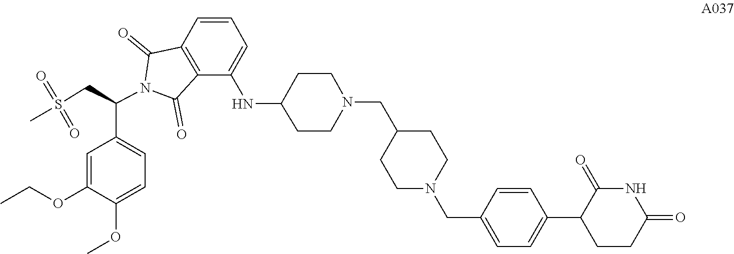 [SMILES: CCOc1cc([C@@H](CS(C)(=O)=O)N2C(=O)c3cccc(NC4CCN(CC5CCN(Cc6ccc(C7CCC(=O)NC7=O)cc6)CC5)CC4)c3C2=O)ccc1OC]

Compound A037 was prepared as shown in Scheme 5.

Preparation of tert-butyl 4-[[4-[[2-[(1S)-1-(3-ethoxy-4-methoxy-phenyl)-2-methylsulfonyl-ethyl]-1,3-dioxo-isoindolin-4-yl]amino]-1-piperidyl]methyl]piperidine-1-carboxylate 5.1. To a solution of 2-[(1S)-1-(3-ethoxy-4-methoxy-phenyl)-2-methylsulfonyl-ethyl]-4-(4-piperidylamino)isoindoline-1,3-dione 4.3 (300 mg, 598 μmol) in DCE (2 mL) were added tert-butyl 4-formylpiperidine-1-carboxylate (128 mg, 598 μmol), NaOAc (98 mg, 1.2 mmol), NaBH₃CN (75.2 mg, 1.2 mmol), and AcOH (36 mg, 598 μmol). After stirred for 3 h, the reaction mixture was concentrated under vacuum, diluted with H₂O and sat. aq. NaHCO₃, and extracted with EtOAc (3×2 mL). The combined organic layers were washed with 5% NaHCO₃, dried over anhydrous Na₂SO₄, and concentrated under reduced pressure to yield a crude product, which was purified by flash silica-gel chromatography eluting with EtOAc/PE to afford compound 5.1 (100 mg) in 22% yield. ¹H NMR (400 MHz, DMSO-d₆) δ 7.59-7.52 (m, 1H), 7.13 (d, J=8.8 Hz, 1H), 7.06 (s, 1H), 7.01 (d, J=7.2 Hz, 1H), 6.94 (s, 2H), 6.25 (d, J=8.0 Hz, 1H), 5.75-5.69 (m, 1H), 4.41-4.32 (m, 1H), 4.13-3.98 (m, 4H), 3.97-3.86 (m, 3H), 3.73 (s, 3H), 3.01 (s, 3H), 2.78-2.65 (m, 5H), 2.21-2.07 (m, 4H), 1.94-1.90 (m, 2H), 1.67 (d, J=11.2 Hz, 3H), 1.53-1.46 (m, 2H), 1.39 (s, 9H), 1.32 (t, J=7.2 Hz, 3H); MS (ESI) m/z: 699.3 [M+H]⁻.

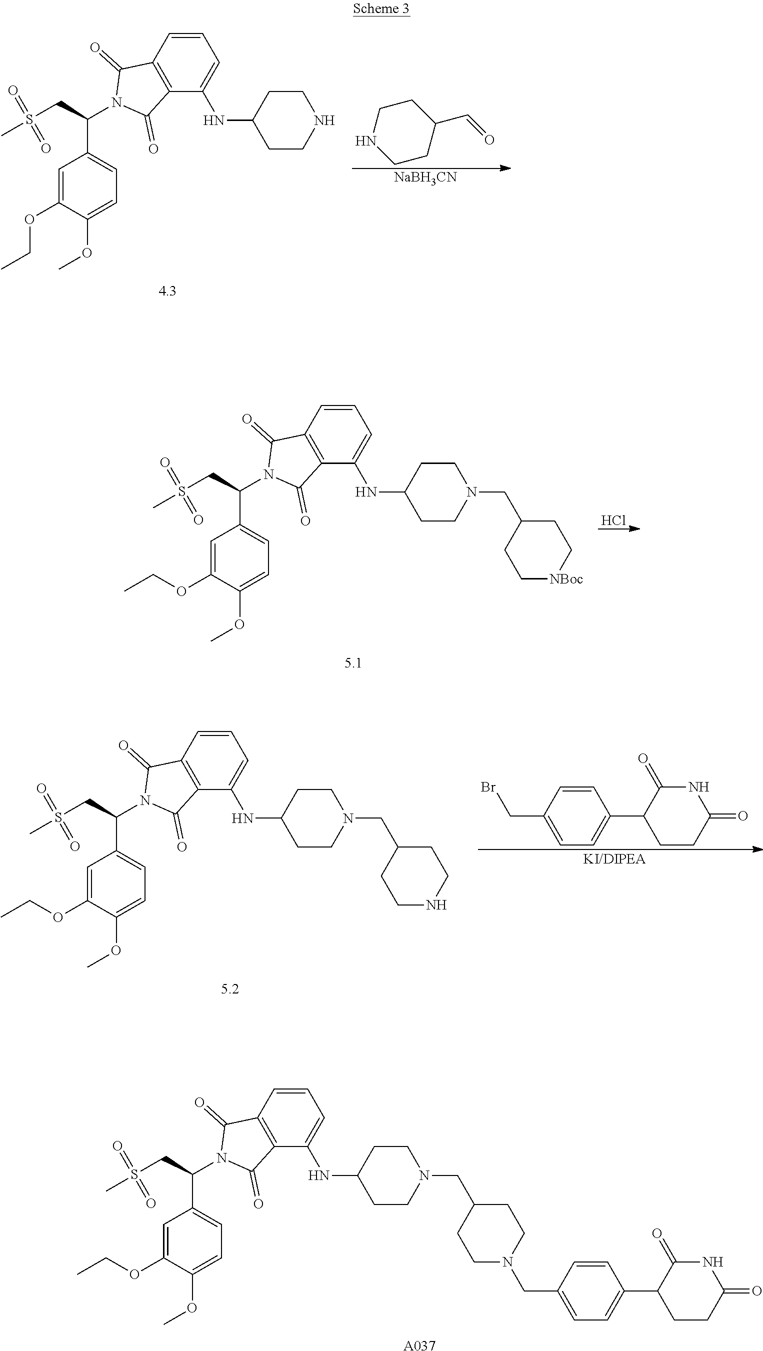

Preparation of 2-[(1S)-1-(3-ethoxy-4-methoxy-phenyl)-2-methylsulfonyl-ethyl]-4-[[1-(4-piperidylmethyl)-4-piperidyl]amino]isoindoline-1,3-dione 5.2. To a solution of compound 5.1 (90 mg, 129 μmol) in DCM (1 mL) was added 4M HCl in dioxane (0.5 mL). After stirred for 1 h, the reaction mixture was concentrated under reduced pressure to afford compound 5.2 (70 mg), which was used directly in the next step without further purification. MS (ESI) m/z: 599.4 [M+H]⁺.

Preparation of 4-((1-((1-(4-(2,6-dioxopiperidin-3-yl)benzyl)piperidin-4-yl)-methyl)piperidin-4-yl)amino)-2-((S)-1-(3-ethoxy-4-methoxyphenyl)-2-(methylsulfonyl)ethyl)-isoindoline-1,3-dione A037. To a solution of compound 5.2 (65 mg, 109 μmol) and 3-[4-(bromo-methyl)phenyl]piperidine-2,6-dione (31 mg, 109 μmol) in DMF (1 mL) were added KI (9 mg, 54 μmol) and DIPEA (28 mg, 217 μmol). After stirred at 80° C. for 5 h, the reaction mixture was filtered and purified by reverse-phase prep-HPLC to afford compound A037 (35 mg) in 38% yield. ¹H NMR (400 MHz, DMSO-d₆) δ 10.83 (s, 1H), 8.17 (s, 1H), 7.55 (t, J=7.6 Hz, 1H), 7.29-7.23 (m, 2H), 7.17 (d, J=8.0 Hz, 2H), 7.12 (d, J=8.4 Hz, 1H), 7.06 (s, 1H), 7.00 (d, J=7.2 Hz, 1H), 6.93 (s, 2H), 6.25 (d, J=8.0 Hz, 1H), 5.75-5.67 (m, 1H), 4.34 (d, J=10.4 Hz, 1H), 4.12-4.06 (m, 1H), 4.04-3.98 (m, 2H), 3.87-3.82 (m, 1H), 3.73 (s, 3H), 3.57 (s, 1H), 3.47 (s, 2H), 3.00 (s, 3H), 2.85-2.79 (m, 2H), 2.78-2.70 (m, 2H), 2.67 (s, 1H), 2.47 (d, J=4.4 Hz, 1H), 2.22-2.09 (m, 5H), 2.06 (s, 1H), 2.01-1.87 (m, 4H), 1.73-1.63 (m, 2H), 1.56-1.42 (m, 3H), 1.32 (t, J=6.8 Hz, 3H), 1.17-1.06 (m, 2H); MS (ESI) m/z: 800.4 [M+H]⁺.

### Example 6

### Preparation of 5-(4-((4-(2,6-dioxopiperidin-3-yl)phenyl)ethynyl)-[1,4′-bipiperidin]-1′-yl)-2-((S)-1-(3-ethoxy-4-methoxyphenyl)-2-(methylsulfonyl)ethyl)isoindoline-1,3-dione A041

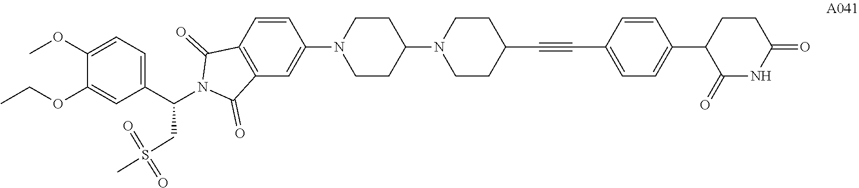 [SMILES: CCOc1cc([C@@H](CS(C)(=O)=O)N2C(=O)c3ccc(N4CCC(N5CCC(C#Cc6ccc(C7CCC(=O)NC7=O)cc6)CC5)CC4)cc3C2=O)ccc1OC]

Compound A041 was prepared as shown in Schemes 6A and 6B.

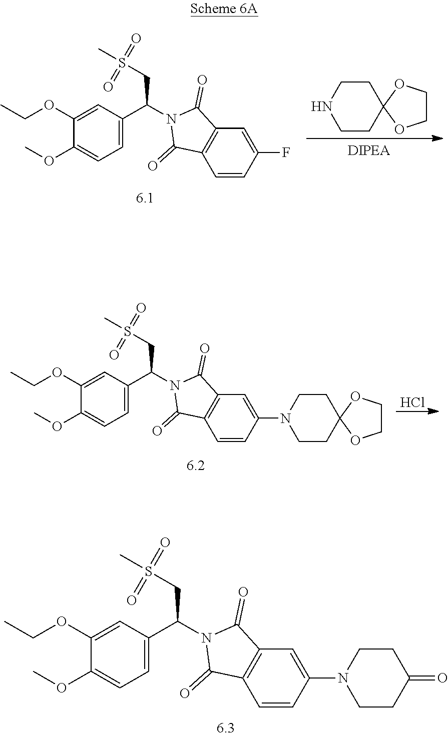 [SMILES: CCOc1cc([C@@H](CS(C)(=O)=O)N2C(=O)c3ccc(F)cc3C2=O)ccc1OC ; C1CC2(CCN1)OCCO2 ; CCOc1cc([C@@H](CS(C)(=O)=O)N2C(=O)c3ccc(N4CCC5(CC4)OCCO5)cc3C2=O)ccc1OC ; Cl ; CCOc1cc([C@@H](CS(C)(=O)=O)N2C(=O)c3ccc(N4CCC(=O)CC4)cc3C2=O)ccc1OC]

Preparation of 5-(1,4-dioxa-8-azaspiro[4.5]decan-8-yl)-2-[(1S)-1-(3-ethoxy-4-methoxyphenyl)-2-methylsulfonylethyl]isoindoline-1,3-dione 6.2. To a solution of 2-[(IS)-1-(3-ethoxy-4-methoxyphenyl)-2-methylsulfonylethyl]-5-fluoroisoindoline-1,3-dione 6.1 (1 g, 2.37 mmol) and 1,4-dioxa-8-azaspiro[4.5]decane (374 mg, 2.61 mmol) in DMSO (10 mL) was added DIPEA (920 mg, 7.12 mmol). After stirred at 100° C. under N₂ for 2 h, the reaction mixture was diluted with H₂O (30 mL) and extracted with EtOAc (30 mL×3). The combined organic layers were washed with NH₄Cl (20 mL×3), dried over anhydrous Na₂SO₄, filtered, and concentrated under reduced pressure to yield a crude product, which was purified by silica-gel column chromatography eluting with EtOAc/PE to afford compound 6.2 (1.14 g) in 88% yield. MS (ESI) m/z: 545.1 [M+H]⁺.

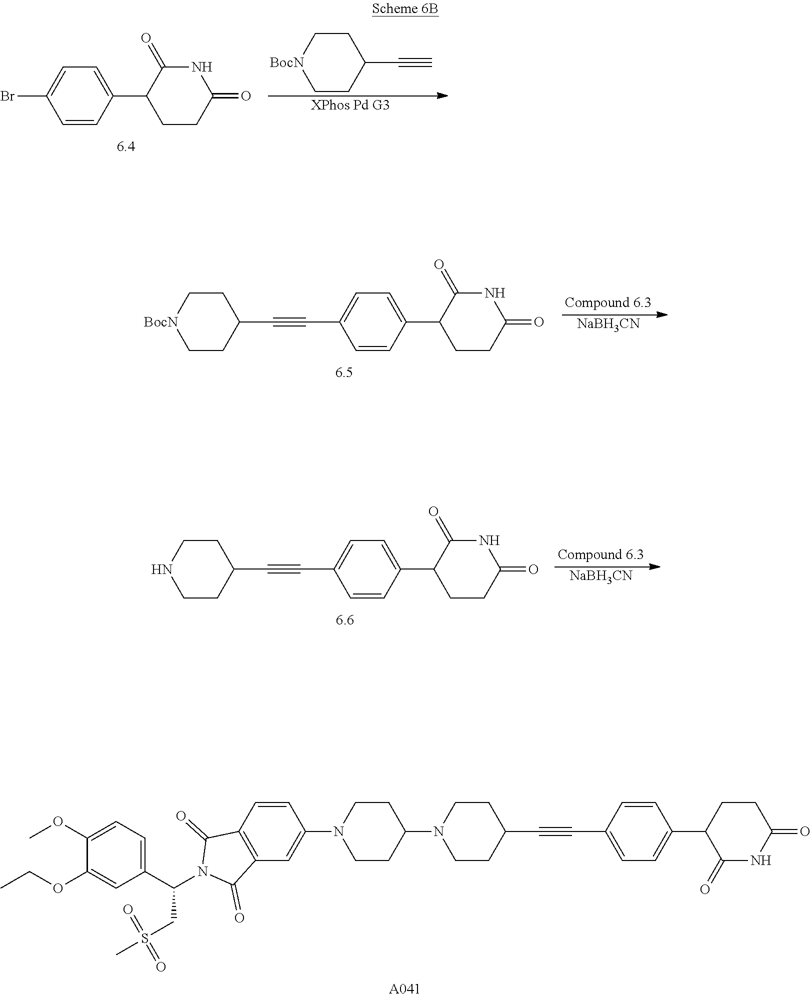

Preparation of 2-[(1S)-1-(3-ethoxy-4-methoxy-phenyl)-2-methylsulfonyl-ethyl]-5-(4-oxo-1-piperidyl)isoindoline-1,3-dione 6.3. A solution of compound 6.2 (1 g, 1.84 mmol) and 4M HCl (10 mL) in THF (10 mL) was stirred at 60° C. for 12 h under N₂. The reaction mixture was neutralized with Na₂CO₃ (20 mL) and extracted with EtOAc (20 mL×3). The combined organic layers were washed with brine (10 mL×3), dried over anhydrous Na₂SO₄, filtered, and concentrated under reduced pressure to afford compound 6.3 (1.08 g), which was used directly in the next step without further purification. MS (ESI) mz: 501.3 [M+H]⁺.

Preparation of tert-butyl 4-[2-[4-(2,6-dioxo-3-piperidyl)phenyl]ethynyl]-piperidine-1-carboxylate 6.5. A solution of 3-(4-bromophenyl)piperidine-2,6-dione 6.4 (500 mg, 1.86 mmol), tert-butyl 4-ethynylpiperidine-1-carboxylate (429 mg, 2.05 mmol), XPhos Pd G3 (158 mg, 186 μmol), and Cs₂CO₃ (1.82 g, 5.59 mmol) in ACN (2.5 mL) and THF (2.5 mL) was stirred at 60° C. under N₂ for 12 h. The reaction mixture was diluted with NH₄Cl (10 mL) and extracted with EtOAc (10 mL×3). The combined organic layers were washed with brine (5 mL×3), dried over anhydrous Na₂SO₄, filtered, and concentrated under reduced pressure to yield a crude product, which was purified by prep-TLC (EtOAc/PE) to afford compound 6.5 (220 mg) in 30% yield. ¹H NMR (400 MHz, DMSO-d₆) δ 10.85 (s, 1H), 7.36 (d, J=8.0 Hz, 2H), 7.20 (d, J=8.4 Hz, 2H), 3.88 (dd, J=4.8, 11.6 Hz, 1H), 3.66-3.60 (m, 2H), 3.14 (dd, J=4.4, 7.2 Hz, 2H), 2.88-2.81 (m, 1H), 2.66 (s, 1H), 2.48-2.44 (m, 1H), 2.25-2.13 (m, 1H), 2.05-1.97 (m, 1H), 1.84-1.77 (m, 2H), 1.53-1.46 (m, 2H), 1.40 (s, 9H).

Preparation of 3-[4-[2-(4-piperidyl)ethynyl]phenyl]piperidine-2,6-dione 6.6. A solution of compound 6.5 (200 mg, 504 μmol) and TFA (280 mg, 2.46 mmol) in DCM (1 mL) was stirred for 30 min. The reaction mixture was concentrated under reduced pressure to afford compound 6.6 (320 mg, TFA), which was used directly in the next step without further purification. MS (ESI) m/z: 297.0 [M+H]⁺.

Preparation of 5-(4-((4-(2,6-dioxopiperidin-3-yl)phenyl)ethynyl)-[1,4′-bipiperidin]-1′-yl)-2-((S)-1-(3-ethoxy-4-methoxyphenyl)-2-(methylsulfonyl)ethyl)isoindoline-1,3-dione A041. To a solution of compound 6.6 (300 mg, 439 μmol, 60% purity, TFA) in MeOH (3 mL) were added NaOAc (180 mg, 2.19 mmol) and 4 A MS (300 mg, 487 μmol), followed by addition of compound 6.3 (268 mg, 536 μmol) at 0° C. After the mixture was stirred for 4 h, NaBH₃CN (33.7 mg, 536 μmol) was added. The reaction mixture was stirred for 12 h, and then diluted with NH₄Cl (2 mL) and extracted with EtOAc (2 mL×3). The combined organic layers were washed with brine (2 mL×3), dried over Na₂SO₄, filtered, and concentrated under reduced pressure to yield a crude product, which was purified by prep-TLC (MeOH/DCM) and further purified by reverse-phase prep-HPLC to afford compound A041 (27 mg) in 7% yield. ¹H NMR (400 MHz, DMSO-d₆) δ 10.84 (s, 1H), 8.21 (s, 1H), 7.60 (d, J=8.4 Hz, 1H), 7.33 (d, J=8.4 Hz, 2H), 7.26 (d, J=2.0 Hz, 1H), 7.23-7.17 (m, 3H), 7.07 (s, 1H), 6.92 (s, 2H), 5.72 (dd, J=4.4, 10.4 Hz, 1H), 4.38-4.31 (m, 1H), 4.10-4.01 (m, 4H), 4.00 (d, J=7.2 Hz, 2H), 3.89-3.85 (m, 1H), 3.72 (s, 3H), 2.98 (s, 3H), 2.97-2.89 (m, 3H), 2.75-2.77 (m, 2H), 2.67 (dd, J=2.0, 3.8 Hz, 1H), 2.62 (br s, 1H), 2.35-2.30 (m, 2H), 2.22-2.16 (m, 1H), 2.04-1.99 (m, 1H), 1.87-1.78 (m, 4H), 1.57 (d, J=10.0 Hz, 2H), 1.50-1.42 (m, 2H), 1.31 (t, J=6.8 Hz, 3H); MS (ESI) m/z: 781.2 [M+H]⁺.

### Example 7

### Preparation of 5-(4-(3-(4-(2,6-dioxopiperidin-3-yl)phenyl)-3-azaspiro[5.5]undecan-9-yl)-piperazin-1-yl)-2-((S)-1-(3-ethoxy-4-methoxyphenyl)-2-(methylsulfonyl)ethyl)isoindoline-1,3-dione A045

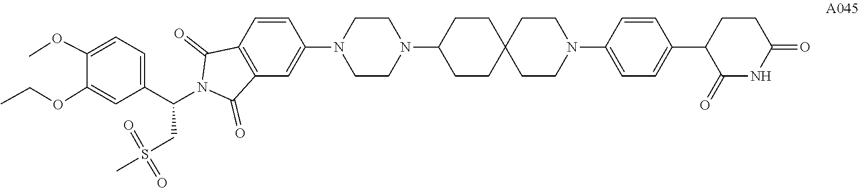 [SMILES: CCOc1cc([C@@H](CS(C)(=O)=O)N2C(=O)c3ccc(N4CCN(C5CCC6(CC5)CCN(c5ccc(C7CCC(=O)NC7=O)cc5)CC6)CC4)cc3C2=O)ccc1OC]

Compound A045 was prepared as shown in Scheme 7.

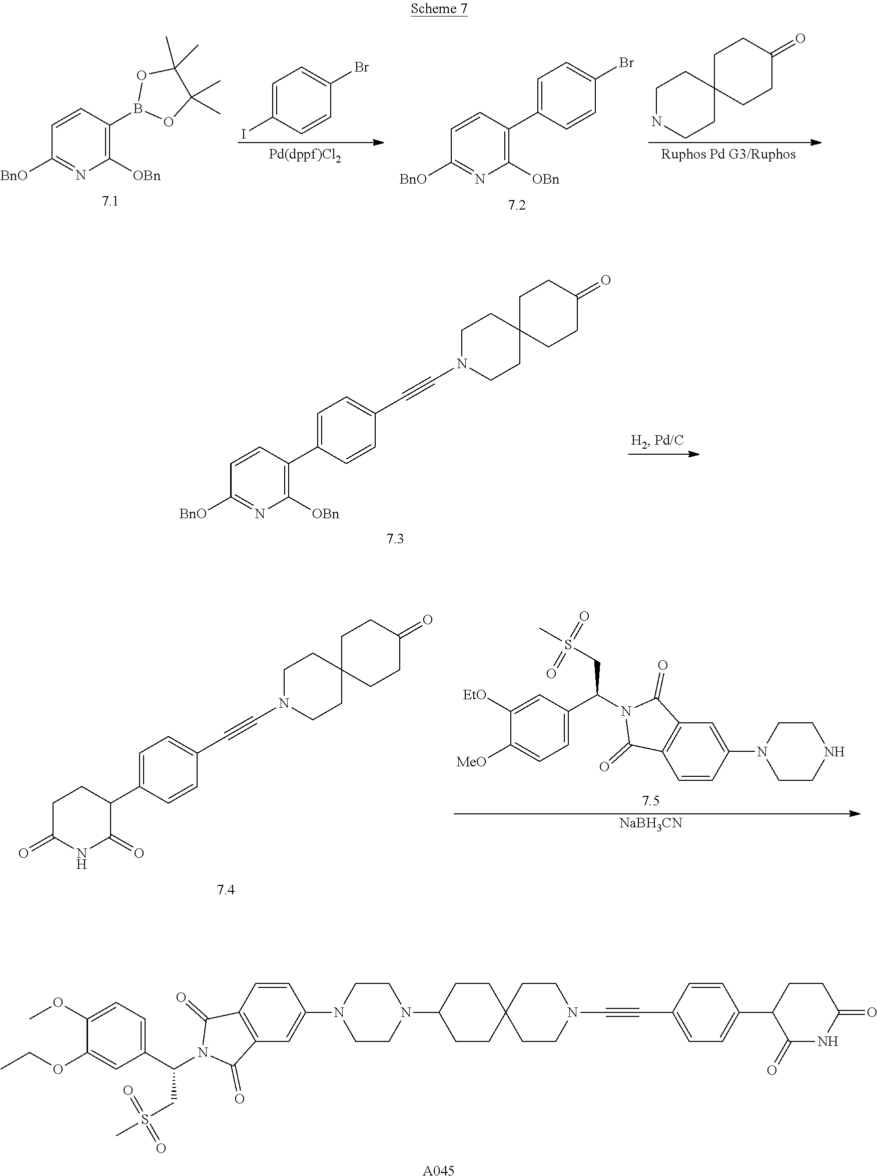

Preparation of 2,6-dibenzyloxy-3-(4-bromophenyl)pyridine 7.2. A mixture of 1-bromo-4-iodo-benzene (1.5 g, 5.3 mmol), 2,6-dibenzyloxy-3-(4,4,5,5-tetramethyl-1,3,2-dioxaborolan-2-yl)pyridine 7.1 (2.32 g, 5.57 mmol), Pd(dppf)Cl₂·CH₂Cl₂ (217 mg, 265 μmol), and K₃PO₄ (1.13 g, 5.30 mmol) in dioxane (10 mL) and H₂O (2 mL) was stirred at 100° C. under N₂ for 16 h. The reaction mixture was diluted with H₂O (20 mL) and extracted with DCM (20 mL×3). The combined organic layers were washed with brine (40 mL), dried with anhydrous Na₂SO₄, filtered, and concentrated under reduced pressure to yield a crude product, which was purified by silica-gel column chromatography eluting with EtOAc/PE to afford compound 7.2 (2.1 g) in 86% yield. ¹H NMR (400 MHz, DMSO-d₆) δ 7.75 (d, J=8.0 Hz, 1H), 7.60-7.55 (m, 2H), 7.54-7.48 (m, 2H), 7.47-7.41 (m, 2H), 7.41-7.28 (m, 8H), 6.56 (d, J=8.0 Hz, 1H), 5.39 (d, J=12.4 Hz, 4H); MS (ESI) m/z: 448.0 [M+H]⁺.

Preparation of 3-[4-(2,6-dibenzyloxy-3-pyridyl)phenyl]-3-azaspiro[5.5]undecan-9-one 7.3. To a solution of compound 7.2 (500 mg, 1.12 mmol) and 3-azaspiro[5.5]undecan-9-one (187 mg, 1.12 mmol) in dioxane (5 mL) were added Cs₂CO₃ (730 mg, 2.24 mmol) and RuPhos Pd G3 (94 mg, 112 μmol). After stirred at 100° C. for 16 h, the reaction mixture was diluted with H₂O (5 mL) and extracted with DCM (5 mL×3). The combine organic layers were washed with brine (10 mL), dried over anhydrous Na₂SO₄, filtered, and concentrated under reduced pressure to yield a crude product, which was purified by flash silica-gel chromatography eluting with EtOAc/PE to afford compound 7.3 (480 mg) in 34% yield. MS (ESI) m/z: 533.3 [M+H]⁺.

Preparation of 3-[4-(9-oxo-3-azaspiro[5.5]undecan-3-yl)phenyl]piperidine-2,6-dione 7.4. To a solution of compound 7.3 (350 mg, 657 μmol) in THF (1.5 mL) and MeOH (1.5 mL) under N₂ was added Pd/C (30 mg, 657 μmol, 10% purity). After hydrogenated at 15 psi of H₂ for 5 h, the reaction mixture was filtered and concentrated under reduced pressure to yield a crude product, which was purified by prep-TLC (EtOAc/PE) to afford compound 7.4 (120 mg) in 50% yield. ¹H NMR (400 MHz, DMSO-d₆) δ 10.77 (s, 1H), 7.05 (d, J=8.8 Hz, 2H), 6.92 (d, J=8.8 Hz, 2H), 3.77-3.63 (m, 1H), 3.23-3.11 (m, 4H), 2.64 (s, 1H), 2.49 (s, 1H), 2.29 (t, J=6.8 Hz, 4H), 2.12 (s, 1H), 2.03 (d, J=5.2 Hz, 1H), 1.79-1.62 (m, 8H); MS (ESI) m/z: 355.0 [M+H]⁺.

Preparation of 5-(4-(3-(4-(2,6-dioxopiperidin-3-yl)phenyl)-3-azaspiro[5.5]-undecan-9-yl)-piperazin-1-yl)-2-((S)-1-(3-ethoxy-4-methoxyphenyl)-2-(methylsulfonyl)ethyl)-isoindoline-1,3-dione A045. To a solution of compound 7.4 (50 mg, 141 μmol) in DMA (1 mL) were added 2-[(1S)-1-(3-ethoxy-4-methoxy-phenyl)-2-methylsulfonyl-ethyl]-5-piperazin-1-yl-isoindoline-1,3-dione 7.5 (68.8 mg, 141 μmol), NaBH₃CN (18 mg, 282 μmol), NaOAc (23 mg, 282 μmol), and AcOH (8.5 mg, 141 μmol). After stirred for 12 h, the reaction mixture was purified by reverse-phase prep-HPLC to afford compound A045 (20 mg) in 16% yield. ¹H NMR (400 MHz, DMSO-d₆) δ 10.77 (s, 1H), 7.05 (d, J=10.8 (s, 1H), 8.20 (s, 1H), 7.63 (d, J=8.4 Hz, 1H), 7.28 (d, J=1.6 Hz, 1H), 7.25-7.20 (m, 1H), 7.08 (s, 1H), 7.03 (d, J=8.8 Hz, 2H), 6.93 (s, 2H), 6.88 (d, J=8.8 Hz, 2H), 5.78-5.65 (m, 1H), 4.42-4.29 (m, 1H), 4.12-4.04 (m, 1H), 4.04-3.97 (m, 2H), 3.73 (s, 3H), 3.39 (d, J=4.4 Hz, 5H), 3.10 (d, J=4.4 Hz, 4H), 3.00 (s, 3H), 2.63 (s, 4H), 2.48-2.42 (m, 2H), 2.27 (s, 1H), 2.11 (s, 1H), 2.04-1.97 (m, 1H), 1.73 (d, J=12.8 Hz, 2H), 1.68-1.57 (m, 4H), 1.47-1.36 (m, 4H), 1.32 (t, J=6.8 Hz, 3H), 1.12 (t, J=11.6 Hz, 2H); MS (ESI) m/z: 826.1 [M+H]⁺.

### Example 8

### Preparation of 5-(4-(1-(4-(4-(2,6-dioxopiperidin-3-yl)phenyl)cyclohexyl)piperidin-4-yl)-piperazin-1-yl)-2-((S)-1-(3-ethoxy-4-methoxyphenyl)-2-(methylsulfonyl)ethyl)isoindoline-1,3-dione A056

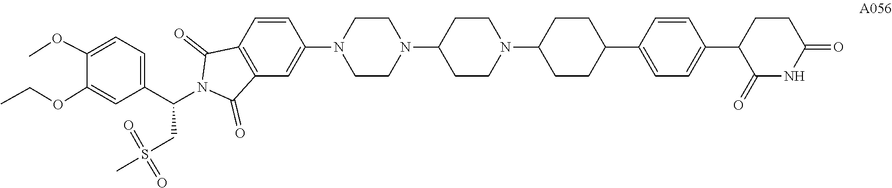 [SMILES: CCOc1cc([C@@H](CS(C)(=O)=O)N2C(=O)c3ccc(N4CCN(C5CCN(C6CCC(c7ccc(C8CCC(=O)NC8=O)cc7)CC6)CC5)CC4)cc3C2=O)ccc1OC]

Compound A056 was prepared as shown in Scheme 8.

Preparation of 8-(4-bromophenyl)-1,4-dioxaspiro[4.5]dec-7-ene 8.2. A mixture of 1-bromo-4-iodo-benzene (5 g, 17.7 mmol), 2-(1,4-dioxaspiro[4.5]dec-7-en-8-yl)-4,4,5,5-tetramethyl-1,3,2-dioxaborolane 8.1 (4.7 g, 17.7 mmol), Pd(dppf)Cl₂·CH₂Cl₂ (722 mg, 884 μmol), and K₃PO₄ (11.25 g, 53 mmol) in dioxane (30 mL) and H₂O (5 mL) were stirred at 80° C. under N₂ for 16 h. The reaction mixture was diluted with H₂O (100 mL) and extracted with EtOAc (150 mL). The combined organic layers were washed with H₂O (150 mL), dried over anhydrous Na₂SO₄, filtered, and concentrated under reduced pressure to yield a crude product, which was purified by silica-gel column chromatography eluting with EtOAc/PE to afford compound 8.2 (4.1 g) in 79% yield. MS (ESI) m/z: 587.1 [M+H]⁺.

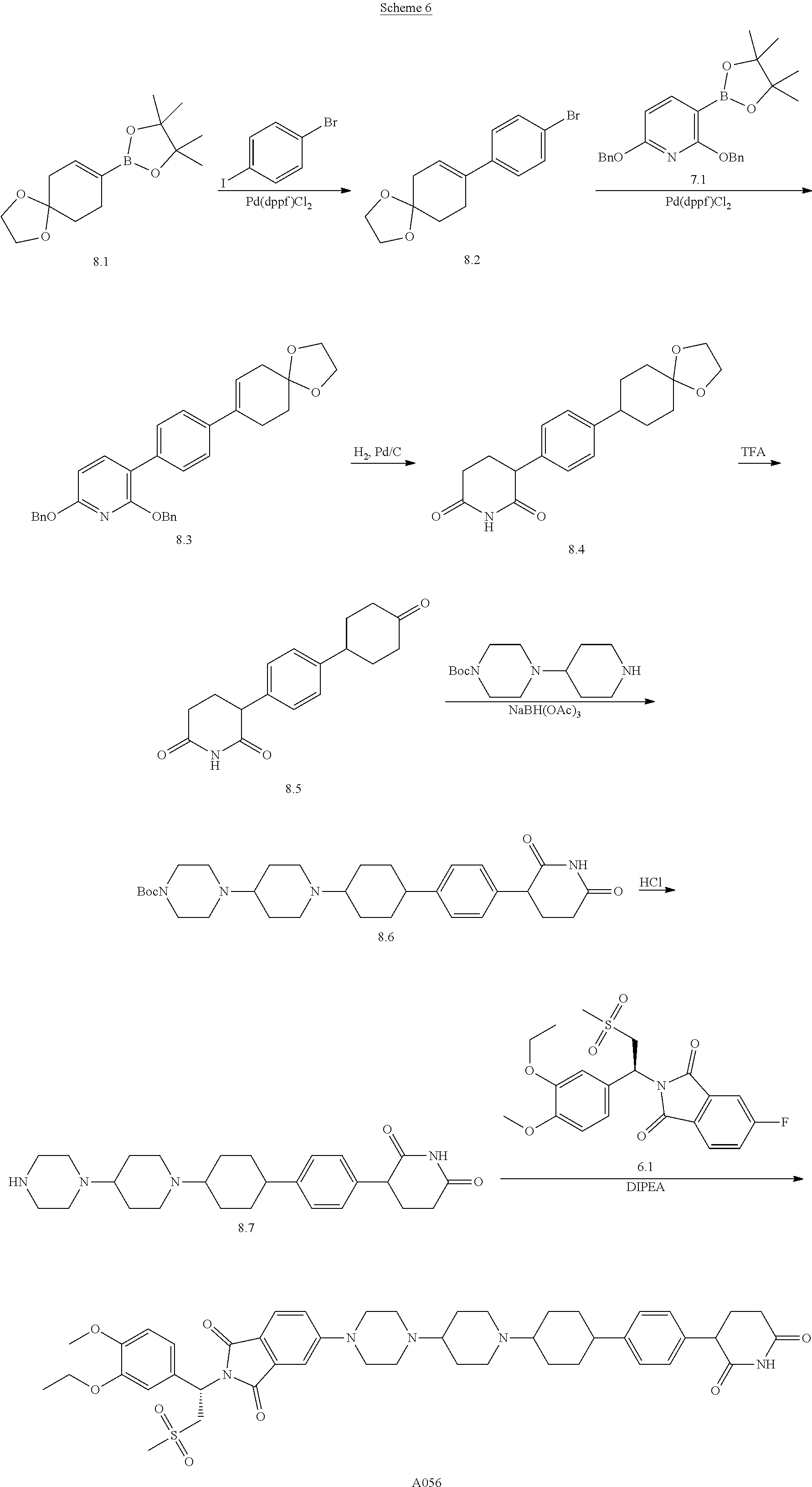

Preparation of 2,6-dibenzyloxy-3-[4-(1,4-dioxaspiro[4.5]dec-7-en-8-yl)phenyl]-pyridine 8.3. A mixture of 2,6-dibenzyloxy-3-(4,4,5,5-tetramethyl-1,3,2-dioxaborolan-2-yl)-pyridine (3.39 g, 8.1 mmol), compound 8.2 (3 g, 10.2 mmol), Pd(dppf)Cl₂·CH₂Cl₂ (415 mg, 508 μmol), and K₃PO₄ (6.47 g, 30.5 mmol) in dioxane (30 mL) and H₂O (5 mL) was stirred at 80° C. under N₂ for 16 h. The reaction mixture was diluted with H₂O (50 mL) and extracted with EtOAc (100 mL). The combined organic layers were washed with brine (150 mL), dried over anhydrous Na₂SO₄, filtered, and concentrated under reduced pressure to yield a crude product, which was purified by silica-gel column chromatography eluting with EtOAc/PE to afford compound 8.3 (2.5 g) in 49% yield. MS (ESI) m/z: 506.2 [M+H]⁺.

Preparation of 3-[4-(1,4-dioxaspiro[4.5]decan-8-yl)phenyl]piperidine-2,6-dione 8.4. To a solution of compound 8.3 (2.2 g, 4.35 mmol) in THF (20 mL) was added Pd/C (500 mg, 4.35 mmol, 10% purity). After hydrogenated at 30 psi of H₂ for 12 h, the reaction mixture was filtered and concentrated under reduced pressure to afford compound 8.4 (1.4 g) in 98% yield, which was used directly in the next step without further purification.

Preparation of 3-[4-(4-oxocyclohexyl)phenyl]piperidine-2,6-dione 8.5. To a solution of compound 8.4 (800 mg, 2.43 mmol) in DCM (6 mL) was added TFA (2 mL). After stirred at 25° C. for 1 h, the reaction mixture was neutralized with sat. NaHCO₃ (50 mL) and extracted with EtOAc (80 mL). The combined organic layers were washed with brine (50 mL), dried over anhydrous Na₂SO₄, filtered, and concentrated under reduced pressure to afford compound 8.5 (640 mg) in 92% yield, which was used directly in the next step without further purification.

Preparation of tert-butyl 4-[1-[4-[4-(2,6-dioxo-3-piperidyl)phenyl]cyclohexyl]-4-piperidyl]piperazine-1-carboxylate 8.6. A mixture of compound 8.5 (594 mg, 2.08 mmol), tert-butyl 4-(4-piperidyl)piperazine-1-carboxylate (510 mg, 1.89 mmol), NaBH(OAc)₃ (803 mg, 3.79 mmol), and NaOAc (466 mg, 5.68 mmol) in MeOH (2 mL) was stirred under N₂ for 16 h. The reaction mixture was diluted with H₂O (50 mL) and extracted with EtOAc (60 mL). The combined organic layers were washed with brine (50 mL), dried over anhydrous Na₂SO₄, filtered, and concentrated under reduced pressure to yield a crude product, which was purified by reverse-phase prep-HPLC to afford compound 8.6 (70 mg) in 7% yield. MS (ESI) m/z: 539.4 [M+H]⁺.

Preparation of 3-[4-[4-(4-piperazin-1-yl-1-piperidyl)cyclohexyl]phenyl]-piperidine-2,6-dione 8.7. To a solution of compound 8.6 (70 mg, 130 μmol) in DCM (3 mL) was added 4M HCl in dioxane (4.2 mL). After stirred for 1 h, the reaction mixture was concentrated under reduced pressure to afford compound 8.7 (50 mg) in 88% yield, which was used directly in the next step without further purification.

Preparation of 5-(4-(1-(4-(4-(2,6-dioxopiperidin-3-yl)phenyl)cyclohexyl)-piperidin-4-yl)-piperazin-1-yl)-2-((S)-1-(3-ethoxy-4-methoxyphenyl)-2-(methylsulfonyl)ethyl)-isoindoline-1,3-dione A056. A mixture of compound 8.7 (40 mg, 91.2 μmol), 2-[(1S)-1-(3-ethoxy-4-methoxy-phenyl)-2-methylsulfonyl-ethyl]-5-fluoro-isoindoline-1,3-dione 6.1 (38.4 mg, 91.2 μmol), and DIPEA (36 mg, 274 μmol) in DMSO (1 mL) was stirred at 100° C. under N₂ for 1 h. The reaction mixture was diluted with H₂O (10 mL) and extracted with EtOAc (10 mL×3). The combined organic layers were washed with brine (50 mL), dried over anhydrous Na₂SO₄, filtered, and concentrated under reduced pressure to yield a crude product, which was purified by silica-gel column chromatography eluting with MeOH/DCM and further purified by reverse-phase prep-HPLC to afford compound A056 (20 mg) in 26% yield. ¹H NMR (400 MHz, DMSO-d₆) δ 10.90-10.81 (m, 1H), 7.62 (d, J=6.8 Hz, 1H), 7.27 (s, 1H), 7.22-7.18 (m, 3H), 7.14-7.12 (m, 2H), 7.06-7.04 (m, 1H), 6.98-6.92 (m, 2H), 5.75-5.71 (m, 1H), 4.35-4.25 (m, 1H), 4.08-3.99 (m, 3H), 3.85-3.80 (m, 1H), 3.72 (s, 3H), 3.40-3.25 (m, 4H), 3.10-3.06 (m, 2H), 2.99 (s, 3H), 2.67-2.61 (m, 6H), 2.50-1.51 (m, 13H), 1.49-1.20 (m, 6H), 1.31 (t, J=6.8 Hz, 3H); MS (ESI) m/z: 840.5 [M+H]⁺.

### Example 9

### Preparation of 4-(5-(4-(4-(2,6-dioxopiperidin-3-yl)phenyl)-[1,4′-bipiperidin]-1′-yl)pentyl)-2-((S)-1-(3-ethoxy-4-methoxyphenyl)-2-(methylsulfonyl)ethyl)isoindoline-1,3-dione A068

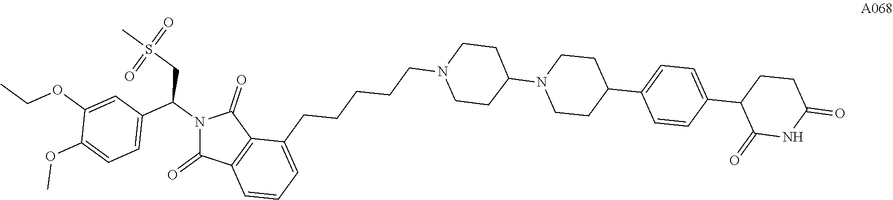 [SMILES: CCOc1cc([C@@H](CS(C)(=O)=O)N2C(=O)c3cccc(CCCCCN4CCC(N5CCC(c6ccc(C7CCC(=O)NC7=O)cc6)CC5)CC4)c3C2=O)ccc1OC]

Compound A068 was prepared as shown in Scheme 9.

Preparation of (S)-4-bromo-2-(1-(3-ethoxy-4-methoxyphenyl)-2-(methyl-sulfonyl)ethyl)isoindoline-1,3-dione 9.1. To a solution of 4-bromoisobenzofuran-1,3-dione (1 g, 3.66 mmol) in AcOH (10 mL) was added (S)-1-(3-ethoxy-4-methoxyphenyl)-2-(methylsulfonyl)-ethanamine (0.83 g, 3.66 mmol). After stirred at 100° C. for 16 h, the reaction mixture was poured into ice-water (30 mL). The resulting precipitates were collected by filtration, washed with water, and dried to afford compound 9.1 (1.1 g) in 51% yield, which was used directly in the next step without further purification. ¹H NMR (400 MHz, DMSO-d₆) δ 8.02 (d, J=8.3 Hz, 1H), 7.89 (d, J=7.3 Hz, 1H), 7.74 (t, J=7.6 Hz, 1H), 7.09 (s, 1H), 7.01-6.92 (m, 2H), 5.79 (dd, J=4.4, 9.8 Hz, 1H), 4.36-4.26 (m, 1H), 4.20-4.13 (m, 1H), 4.02 (q, J=6.8 Hz, 2H), 3.73 (s, 3H), 3.00 (s, 3H), 1.32 (t, J=6.8 Hz, 3H).

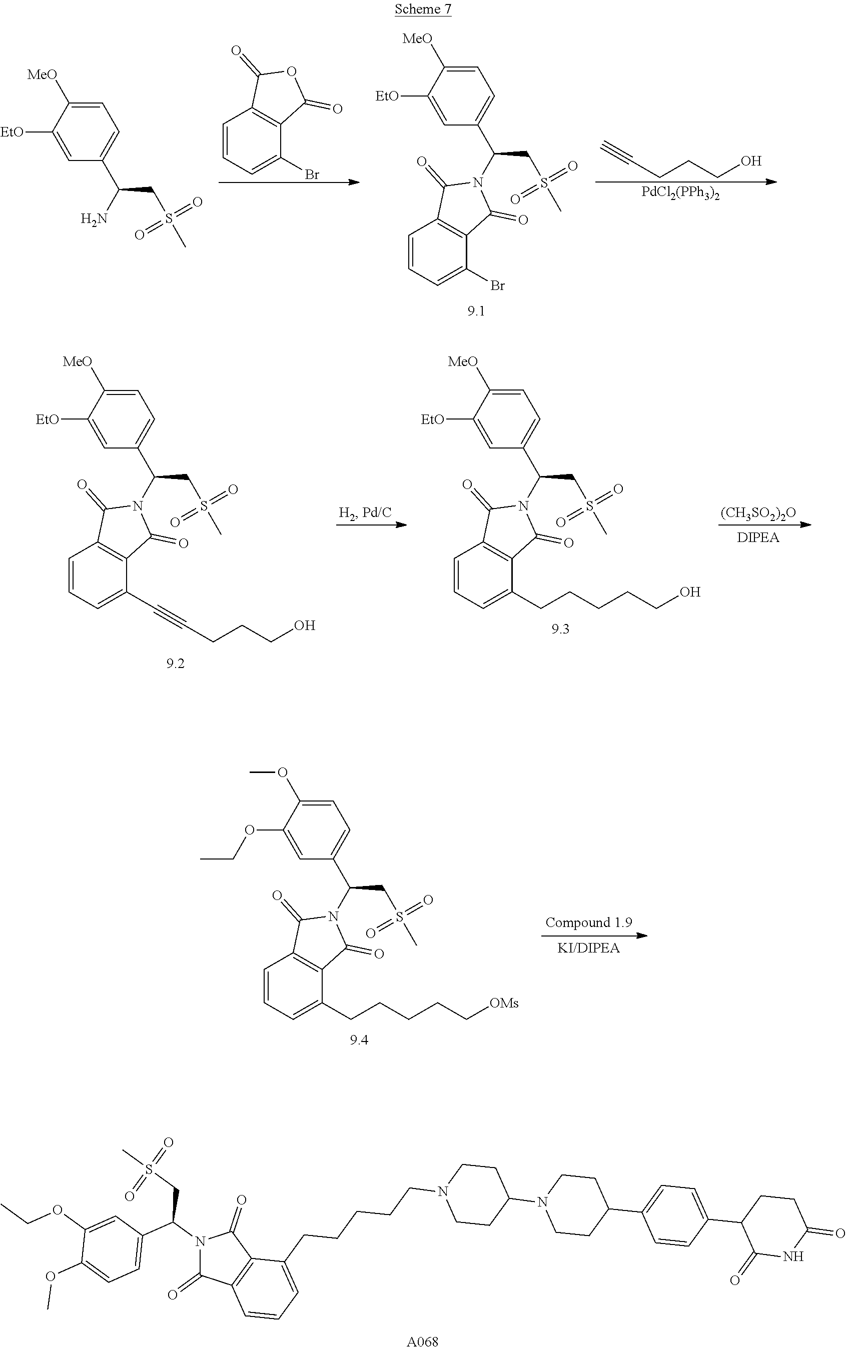

Preparation of(S)-2-(1-(3-ethoxy-4-methoxyphenyl)-2-(methylsulfonyl)ethyl)-4-(5-hydroxypent-1-yn-1-yl)isoindoline-1,3-dione 9.2. To a solution of compound 9.1 (0.5 g, 1.03 mmol) and pent-4-yn-1-ol (0.088 g, 1.03 mmol) in THF (5 mL) under N₂ were added DIPEA (1.34 g, 10.3 mmol), CuI (40 mg, 0.2 mmol), and PdCl₂(PPh₃)₂ (72 mg, 0.1 mmol). After heated at 80° C. for 16 h, the reaction mixture was diluted with water (30 mL) and extracted with EtOAc (2×50 mL). The combined organic layers were washed with brine (30 mL), dried over anhydrous Na₂SO₄, and concentrated under reduced pressure to yield a crude product, which was purified by silica-gel column chromatography eluting with EtOAc/heptane to afford compound 9.2 (0.3 g) in 61% yield.

Preparation of(S)-2-(1-(3-ethoxy-4-methoxyphenyl)-2-(methylsulfonyl)ethyl)-4-(5-hydroxypentyl)isoindoline-1,3-dione 9.3. To a solution of compound 9.2 (0.3 g, 0.63 mmol) in MeOH (10 mL) under N₂ was added 10% Pd/C (0.3 g). After hydrogenated at 100 psi of H₂ for 16 h, the reaction mixture was filtered through celite and the celite was washed with MeOH (20 mL). The organic phase was concentrated under reduced pressure to yield a crude product, which was purified by silica-gel column chromatography eluting with EtOAc/heptane to afford compound 9.3 (0.25 g) in 82% yield. MS (ESI) m/z: 490.4 [M+H]⁺.

Preparation of(S)-5-(2-(1-(3-ethoxy-4-methoxyphenyl)-2-(methylsulfonyl)ethyl)-1,3-dioxoisoindolin-4-yl)pentyl methanesulfonate 9.4. To a solution of compound 9.3 (0.1 g, 0.21 mmol) in DCM (5 mL) at 0° C. were added methanesulfonic anhydride (33 mg, 0.83 mmol) and DIPEA (0.36 mL, 2.1 mmol). After stirred for 12 h, the reaction mixture was diluted with water (30 mL) and extracted with 5% MeOH in DCM (2×20 mL). The combined organic layers were washed with brine (30 mL), dried over anhydrous Na₂SO₄, and concentrated under reduced pressure to afford compound 9.4 (0.1 g) in 84% yield, which was used directly in the next step without further purification.

Preparation of 4-(5-(4-(4-(2,6-dioxopiperidin-3-yl)phenyl)-[1,4′-bipiperidin]-1′-yl)pentyl)-2-((S)-1-(3-ethoxy-4-methoxyphenyl)-2-(methylsulfonyl)ethyl)isoindoline-1,3-dione A068. To a solution of compound 9.4 (0.11 g, 0.19 mmol) in DMF (2 mL) at 0° C. were added DIPEA (0.17 mL, 0.97 mmol), KI (0.322 g, 1.94 mmol), and 3-(4-([1,4′-bipiperidin]-4-yl)-phenyl)piperidine-2,6-dione hydrochloride 1.9 (76 mg, 0.194 mmol,). After stirred at 80° C. for 12 h, the reaction mixture was diluted with ice-water (20 mL) and extracted with EtOAc (2×30 mL). The combined organic layers were washed with brine (20 mL), dried over anhydrous Na₂SO₄, and concentrated under reduced pressure to yield a crude product, which was purified by reverse-phase prep-HPLC to afford compound A068 (40 mg) in 25% yield. ¹H NMR (400 MHz, DMSO-d₆) δ 10.80 (s, 1H), 9.93-9.84 (bs, 1H), 9.68-9.57 (br s, 1H) 7.79-7.71 (m, 2H), 7.67 (d, J=6.8 Hz, 1H), 7.20 (s, 4H), 7.09 (s, 1H), 6.99-6.92 (m, 2H), 5.79 (d, J=3.2, 10.0 Hz, 1H), 4.41-4.30 (m, 1H), 4.17-4.10 (m, 1H), 4.02 (q, J=6.7 Hz, 2H), 3.84 (dd, J=4.6, 11.0 Hz, 2H), 3.74 (s, 3H), 3.65-3.63 (s, 2H), 3.58 (d, J=5.9 Hz, 2H), 3.14-3.06 (m, 2H), 3.05 (d, J=6.8 Hz, 3H), 3.01 (s, 3H), 2.98-2.92 (m, 2H), 2.91-2.82 (m, 2H), 2.76-2.60 (m, 2H), 2.31 (d, J=11.7 Hz, 2H), 2.23-2.13 (m, 2H), 2.10-2.01 (m, 3H), 1.95-1.92 (s, 3H), 1.74-1.59 (m, 4H), 1.40-1.28 (m, 5H); MS (ESI) m/z: 828.0 [M+H]⁺.

### Example 10

### Preparation of 4-(11-(4-(4-(2,6-dioxopiperidin-3-yl)phenyl)piperidin-1-yl)undecyl)-2-((S)-1-(3-ethoxy-4-methoxyphenyl)-2-(methylsulfonyl)ethyl)isoindoline-1,3-dione A076

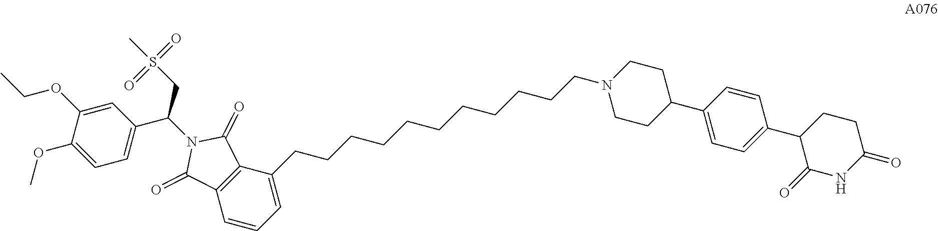 [SMILES: CCOc1cc([C@@H](CS(C)(=O)=O)N2C(=O)c3cccc(CCCCCCCCCCCN4CCC(c5ccc(C6CCC(=O)NC6=O)cc5)CC4)c3C2=O)ccc1OC]

Compound A076 was prepared as shown in Scheme 10.

Preparation of(S)-2-(1-(3-ethoxy-4-methoxyphenyl)-2-(methylsulfonyl)ethyl)-4-(11-hydroxyundec-1-yn-1-yl)isoindoline-1,3-dione 10.2. To a solution of compound 9.1 (1 g, 2.07 mmol) and undec-10-yn-1-ol (0.35 g, 2.07 mmol) in DMF (10 mL) under N₂ were added TEA (1.43 mL, 10.4 mmol), CuI (78 mg, 0.414 mmol), and PdCl₂(PPh₃)₂ (0.15 g, 0.207 mmol). After heated at 80° C. for 16 h, the reaction mixture was diluted with water (50 mL) and extracted with EtOAc (2×100 mL). The combined organic layers were washed with brine (50 mL), dried over anhydrous Na₂SO₄, filtered, and concentrated under reduced pressure to yield a crude product, which was purified by silica-gel column chromatography eluting with EtOAc/heptane to afford compound 8.2 (0.8 g) in 67% yield. MS (ESI) m/z: 570.4 [M+H]⁺.

Preparation of(S)-2-(1-(3-ethoxy-4-methoxyphenyl)-2-(methylsulfonyl)ethyl)-4-(11-hydroxyundecyl)isoindoline-1,3-dione 10.3. To a solution of compound 10.2 (1 g, 1.75 mmol) in MeOH (20 mL) under N₂ was added 10% Pd/C (1 g). After hydrogenated at 100 psi of H₂ for 16 h, the reaction mixture was filtered through celite and the celite was washed with MeOH (20 mL). The organic phase was concentrated under reduced pressure to afford compound 10.3 (0.7 g) in 69% yield, which was used directly in the next step without further purification. MS (ESI) m/z: 574.6 [M+H]⁺.

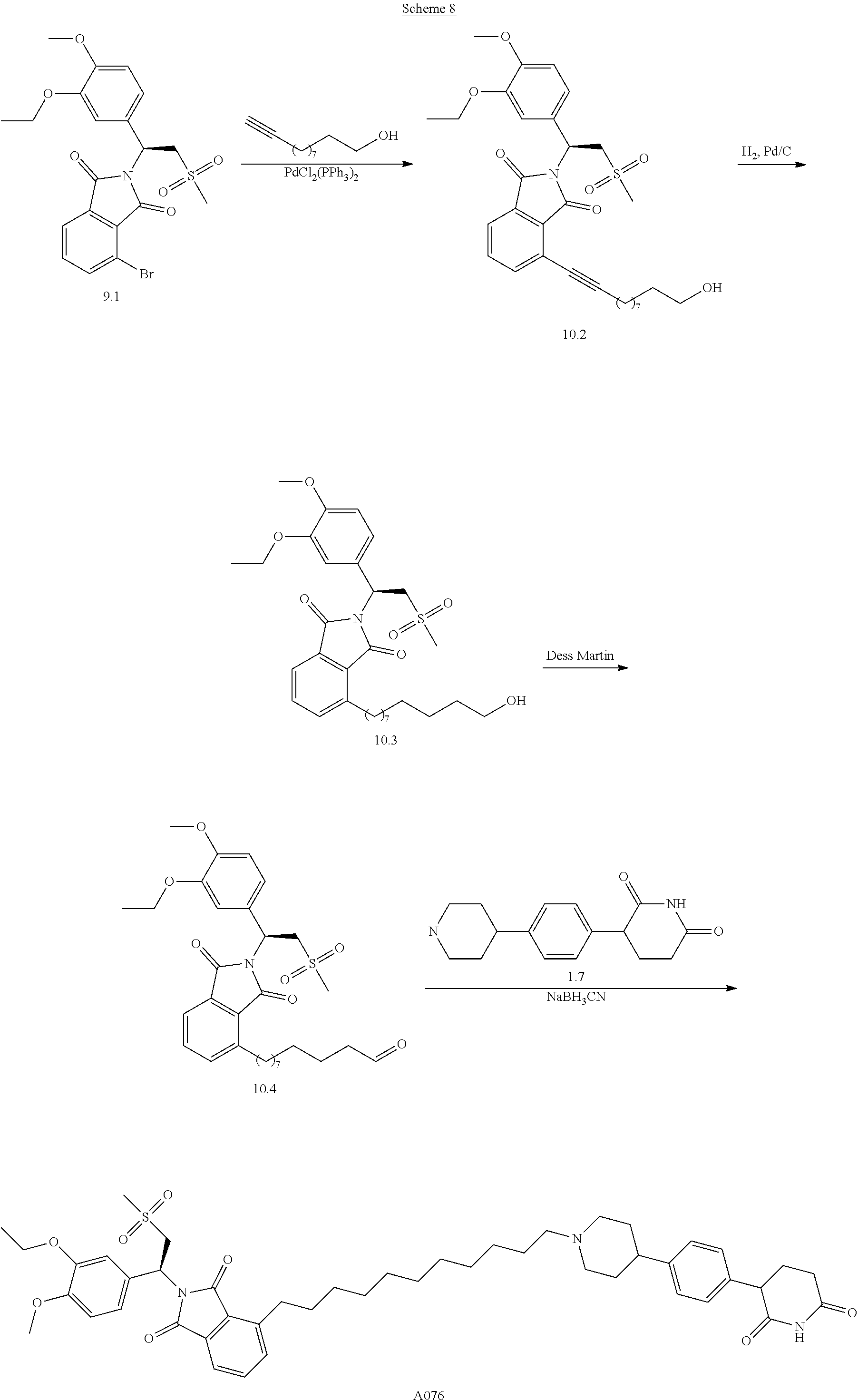

Preparation of ((S)-11-(2-(1-(3-ethoxy-4-methoxyphenyl)-2-(methylsulfonyl) ethyl)-1,3-dioxoisoindolin-4-yl) undecanal 10.4. To a solution of compound 10.3 (0.75 g, 1.3 mmol) in DCM (10 mL) was added the Dess-Martin periodinane (1.1 g, 2.61 mmol). After stirred for 12 h, the reaction mixture was diluted with water (20 mL) and extracted with DCM (2×50 mL). The combined organic layers were washed with brine (50 mL), dried over anhydrous Na₂SO₄, and concentrated under reduced pressure to afford compound 10.4 (0.6 g), which was used directly in the next step without further purification.

Preparation of 4-(11-(4-(4-(2,6-dioxopiperidin-3-yl)phenyl)piperidin-1-yl)-undecyl)-2-((S)-1-(3-ethoxy-4-methoxyphenyl)-2-(methylsulfonyl)ethyl)isoindoline-1,3-dione A076. To a solution of compound 10.4 (0.3 g, 0.525 mmol) in DCM (3 mL) and EtOH (3 mL) were added NaOAc (0.082 g, 0.997 mmol) and 3-(4-(piperidin-4-yl)phenyl)piperidine-2,6-dione hydrochloride 1.7 (0.14 g, 0.525 mmol). After the mixture was stirred for 20 min, NaBH₃CN (0.062 g, 0.997 mmol) was added. The reaction mixture was stirred for 16 h, and then diluted with water (20 mL) and extracted with EtOAc (2×50 mL). The combined organic layers were washed with brine (50 mL), dried over anhydrous Na₂SO₄, and concentrated under reduced pressure to yield a crude product, which was purified by reverse-phase prep-HPLC to afford compound A076 (27.5 mg) in 6.3% yield as an off white solid. ¹H NMR (400 MHz, DMSO-d₆) δ 10.82 (s, 1H), 9.03 (br s, 1H), 7.74-7.69 (m, 2H), 7.65 (dd, J=1.6, 7.1 Hz, 1H), 7.20 (s, 3H), 7.09 (d, J=1.8 Hz, 1H), 6.97 (d, J=1.6 Hz, 1H), 6.96-6.93 (m, 1H), 5.77 (dd, J=4.4, 10.3 Hz, 1H), 4.36 (dd, J=10.4, 14.4 Hz, 1H), 4.12 (dd, J=4.6, 14.3 Hz, 1H), 4.04-3.99 (m, 2H), 3.83 (dd, J=4.9, 11.5 Hz, 1H), 3.73 (s, 3H), 3.57 (d, J=12.8 Hz, 2H), 3.08-3.01 (m, 4H), 2.99 (s, 3H), 2.81 (t, J=3.5 Hz, 1H), 2.68-2.65 (m, 1H), 2.65-2.64 (m, 1H), 2.17 (dd, J=3.5, 12.5 Hz, 1H), 2.04-2.00 (m, 2H), 1.83 (d, J=10.8 Hz, 2H), 1.67-1.55 (m, 5H), 1.33 (s, 1H), 1.31 (s, 3H), 1.28 (m, 15H); MS (ESI) mi: 828.9 [M+H]⁺.

### Example 11

### Preparation of 5-(4-((4-((4-(4-((2,6-dioxopiperidin-3-yl)amino)phenyl)piperidin-1-yl)methyl)-piperidin-1-yl)methyl)piperidin-1-yl)-2-((S)-1-(3-ethoxy-4-methoxyphenyl)-2-(methylsulfonyl)-ethyl)isoindoline-1,3-dione A086

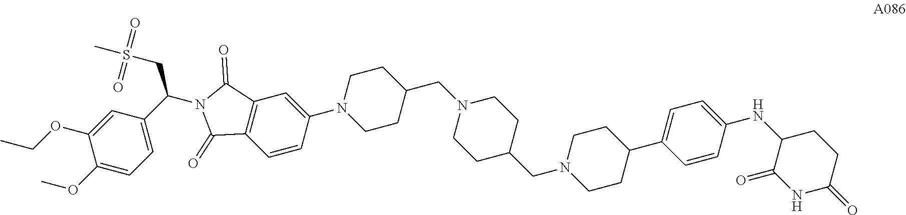 [SMILES: CCOc1cc([C@@H](CS(C)(=O)=O)N2C(=O)c3ccc(N4CCC(CN5CCC(CN6CCC(c7ccc(NC8CCC(=O)NC8=O)cc7)CC6)CC5)CC4)cc3C2=O)ccc1OC]

Compound A086 was prepared as shown in Scheme 11.

Preparation of tert-butyl 4-(4-((2,6-dioxopiperidin-3-yl)amino)phenyl)piperidine-1-carboxylate 11.2. To a solution of 3-bromopiperidine-2,6-dione (1.04 g, 5.43 mmol) and tert-butyl 4-(4-aminophenyl)piperidine-1-carboxylate 11.1 (1 g, 3.62 mmol) in DMF (10 mL) was added NaHCO₃ (912 mg, 10.9 mmol). After stirred at 65° C. for 16 h, the reaction mixture was diluted with H₂O (10 mL) and extracted with EtOAc (10 mL×3). The combined organic layers were washed with brine (10 mL×3), dried over anhydrous Na₂SO₄, filtered, and concentrated under reduced pressure to yield a crude product, which was purified by silica-gel column chromatography eluting with EtOAc/PE to afford compound 11.2 (1.1 g) in 77% yield. MS (ESI) m/z: 387.2 [M+H]⁺.

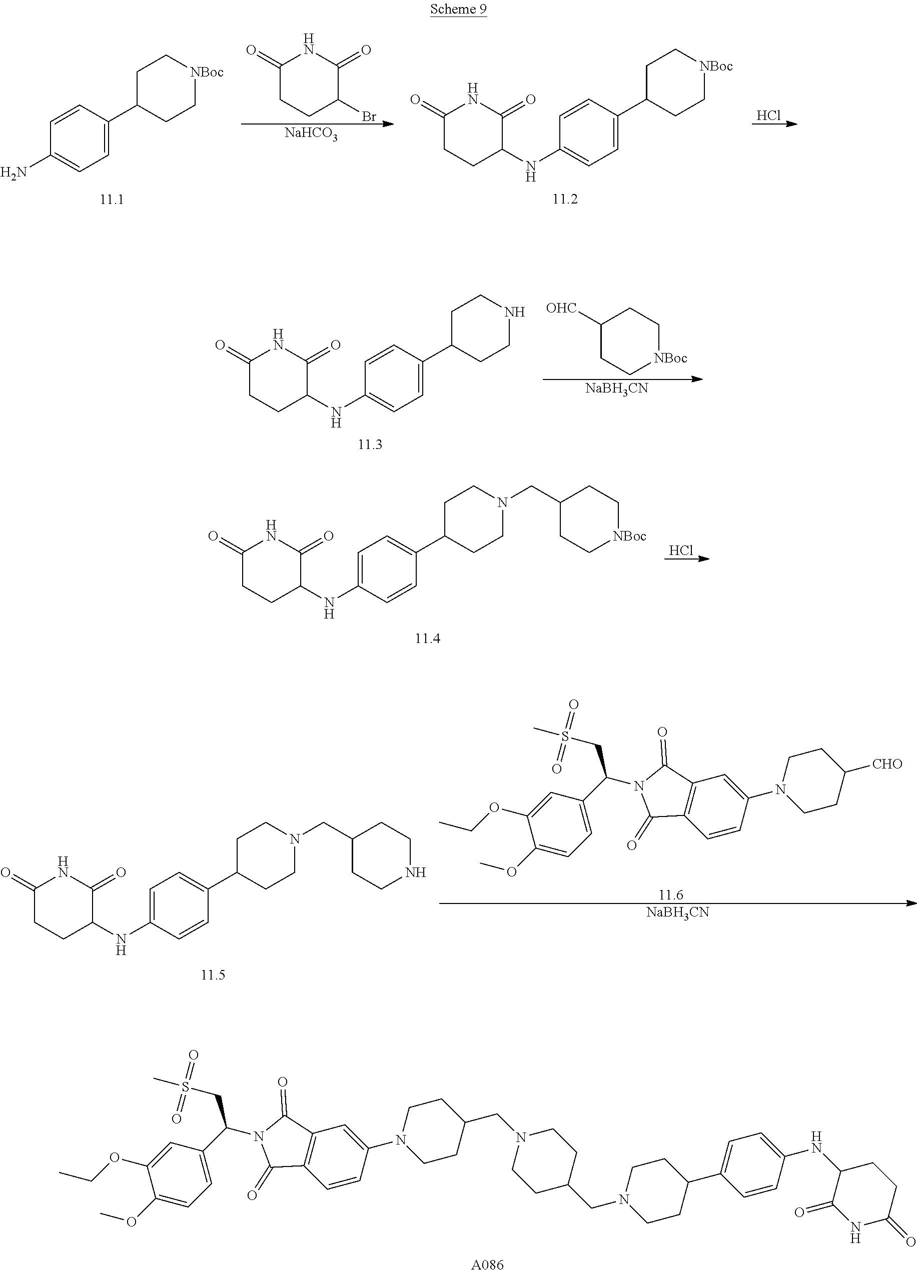

Preparation of 3-((4-(piperidin-4-yl)phenyl)amino)piperidine-2,6-dione 11.3. To a solution of compound 11.2 (150 mg, 387 μmol) in DCM (0.75 mL) was added 4M HCl in dioxane (97 μL). After stirred for 2 h, the reaction mixture was concentrated under reduced pressure to afford compound 11.3 (140 mg, HCl), which was used directly in the next step without further purification. MS (ESI) m/z: 287.4 [M+H]⁺.

Preparation of tert-butyl 4-((4-(4-((2,6-dioxopiperidin-3-yl)amino)phenyl)-piperidin-1-yl)methyl)piperidine-1-carboxylate 11.4. To a solution of compound 11.3 (100 mg, 348 μmol) in MeOH (1 mL) was added NaOAc (85.6 mg, 1.04 mmol), followed by addition of tert-butyl 4-formylpiperidine-1-carboxylate (148 mg, 696 μmol) at 0° C. After the mixture was stirred for 10 min, NaBH₃CN (32.8 mg, 522 μmol) was added. The reaction mixture was stirred for 12 h, diluted with H₂O (2 mL), and extracted with EtOAc (2 mL×3). The combined organic layers were washed with brine (2 mL×3), dried over anhydrous Na₂SO₄, filtered, and concentrated under reduced pressure to yield a crude product, which was purified by silica-gel column chromatography eluting with EtOAc/PE to afford compound 11.4 (130 mg) in 67% yield. MS (ESI) m/z: 484.6 [M+H]⁺.

Preparation of 3-((4-(1-(piperidin-4-ylmethyl)piperidin-4-yl)phenyl)amino)-piperidine-2,6-dione 11.5. To a solution of compound 11.4 (130 mg, 268 μmol) in DCM (1.5 mL) was added 4M HCl in dioxane (1.3 mL). After stirred for 1 h, the reaction mixture was concentrated under reduced pressure to afford compound 11.5 (100 mg, HCl), which was used directly in the next step without further purification. MS (ESI) m/z: 384.5 [M+H]⁺.

Preparation of 5-(4-((4-((4-(4-((2,6-dioxopiperidin-3-yl)amino)phenyl)piperidin-1-yl)methyl)-piperidin-1-yl)methyl)piperidin-1-yl)-2-((S)-1-(3-ethoxy-4-methoxyphenyl)-2-(methylsulfonyl)-ethyl)isoindoline-1,3-dione A086. To a solution of compound 11.5 (100 mg, 260 μmol) in MeOH (1 mL) was added NaOAc (42.7 mg, 520 μmol), followed by addition of (S)-1-(2-(1-(3-ethoxy-4-methoxyphenyl)-2-(methylsulfonyl)ethyl)-1,3-dioxoisoindolin-5-yl)-piperidine-4-carbaldehyde 11.6 (89.2 mg, 173 μmol) at 0° C. After the mixture was stirred for 10 min, NaBH₃CN (16.3 mg, 260 μmol) was added. The reaction mixture was stirred for 10 h and concentrated under reduced pressure to yield a crude product, which was purified by reverse-phase prep-HPLC to afford compound A086 (16.6 mg) in 11% yield. ¹H NMR (400 MHz, DMSO-d₆) δ 10.79-10.75 (m, 1H), 8.22 (s, 1H), 7.63-7.56 (m, 1H), 7.24 (s, 1H), 7.21-7.16 (m, 1H), 7.07 (s, 1H), 6.96-6.90 (m, 4H), 6.59 (d, J=8.4 Hz, 2H), 5.72-5.77 (m, 1H), 5.64 (br d, J=7.2 Hz, 1H), 4.39-4.23 (m, 2H), 4.09-3.97 (m, 5H), 3.72 (s, 3H), 2.99 (s, 3H), 2.94-2.87 (m, 4H), 2.83-2.78 (m, 2H), 2.11-2.15 (m, 5H), 1.93-1.74 (m, 9H), 1.66-1.69 (m, 4H), 1.60-1.42 (m, 4H), 1.31 (t, J=6.8 Hz, 3H), 1.14-1.04 (m, 4H); MS (ESI) m/z: 883.1 [M+H]⁺.

### Example 12

### Preparation of 4-(1′-(4-((3-(2,6-dioxopiperidin-3-yl)-1-methyl-1H-indazol-6-yl)oxy)benzyl)-[4,4′-bipiperidin]-1-yl)-2-((S)-1-(3-ethoxy-4-methoxyphenyl)-2-(methylsulfonyl)ethyl)-isoindoline-1,3-dione B003

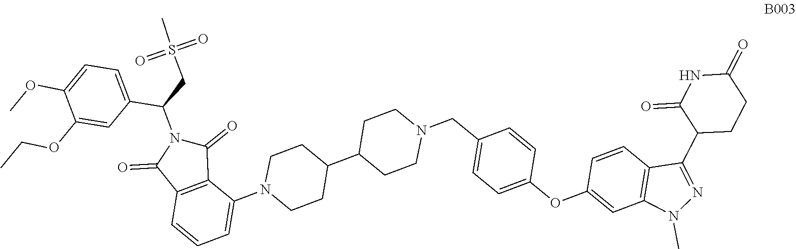 [SMILES: CCOc1cc([C@@H](CS(C)(=O)=O)N2C(=O)c3cccc(N4CCC(C5CCN(Cc6ccc(Oc7ccc8c(C9CCC(=O)NC9=O)nn(C)c8c7)cc6)CC5)CC4)c3C2=O)ccc1OC]

Compound B003 was prepared as shown in Scheme 12.

Preparation of 3-[6-[4-(bromomethyl)phenoxy]-1-methylindazol-3-yl]piperidine-2,6-dione 12.2. To a solution of 3-[6-[4-(hydroxymethyl)phenoxy]-1-methylindazol-3-yl]-piperidine-2,6-dione 12.1 (500 mg, 1.37 mmol) in ACN (10 mL) was added PBr₃ (370 mg, 1.37 mmol). After stirred at 80° C. for 2 h, the reaction mixture was diluted with H₂O (10 mL) and then with EtOAc (10 mL), and extracted with EtOAc (20 mL×3). The combined organic layers were washed with saturated aqueous NaHCO₃, dried over anhydrous Na₂SO₄, filtered, and concentrated under reduced pressure to afford compound 12.2 (360 mg), which was used directly in the next step without further purification.

Preparation of tert-butyl 4-[1-[2-[(1S)-1-(3-ethoxy-4-methoxyphenyl)-2-methylsulfonylethyl]-1,3-dioxoisoindolin-4-yl]-4-piperidyl]piperidine-1-carboxylate 12.4. To a solution of 2-[(1S)-1-(3-ethoxy-4-methoxyphenyl)-2-methylsulfonylethyl]-4-fluoroisoindoline-1,3-dione 12.3 (1.65 g, 3.92 mmol) in DMSO (20 mL) were added DIPEA (1.44 g, 11.2 mmol) and tert-butyl 4-(4-piperidyl)piperidine-1-carboxylate (1 g, 3.73 mmol). After stirred at 100° C. for 12 h, the reaction mixture was diluted with H₂O (100 mL) and extracted with EtOAc (100 mL). The combined organic layers were washed with brine (100 mL), dried over anhydrous Na₂SO₄, filtered, and concentrated under reduced pressure to afford compound 12.4 (2.52 g) in a quantitative yield. MS (ESI) m/z: 670.5 [M+H]⁺.

Preparation of 2-[(1S)-1-(3-ethoxy-4-methoxyphenyl)-2-methylsulfonylethyl]-4-[4-(4-piperidyl)-1-piperidyl]isoindoline-1,3-dione 12.5. To a solution of compound 12.4 (2.52 g, 3.76 mmol) in DCM (10 mL) was added 4M HCl in dioxane (10 mL). After stirred at 20° C. for 2 h, the reaction mixture was concentrated under reduced pressure to afford compound 12.5 (2.4 g) in 98% yield, which was used directly in the next step without further purification. MS (ESI) m/z: 570.6 [M+H]⁺.

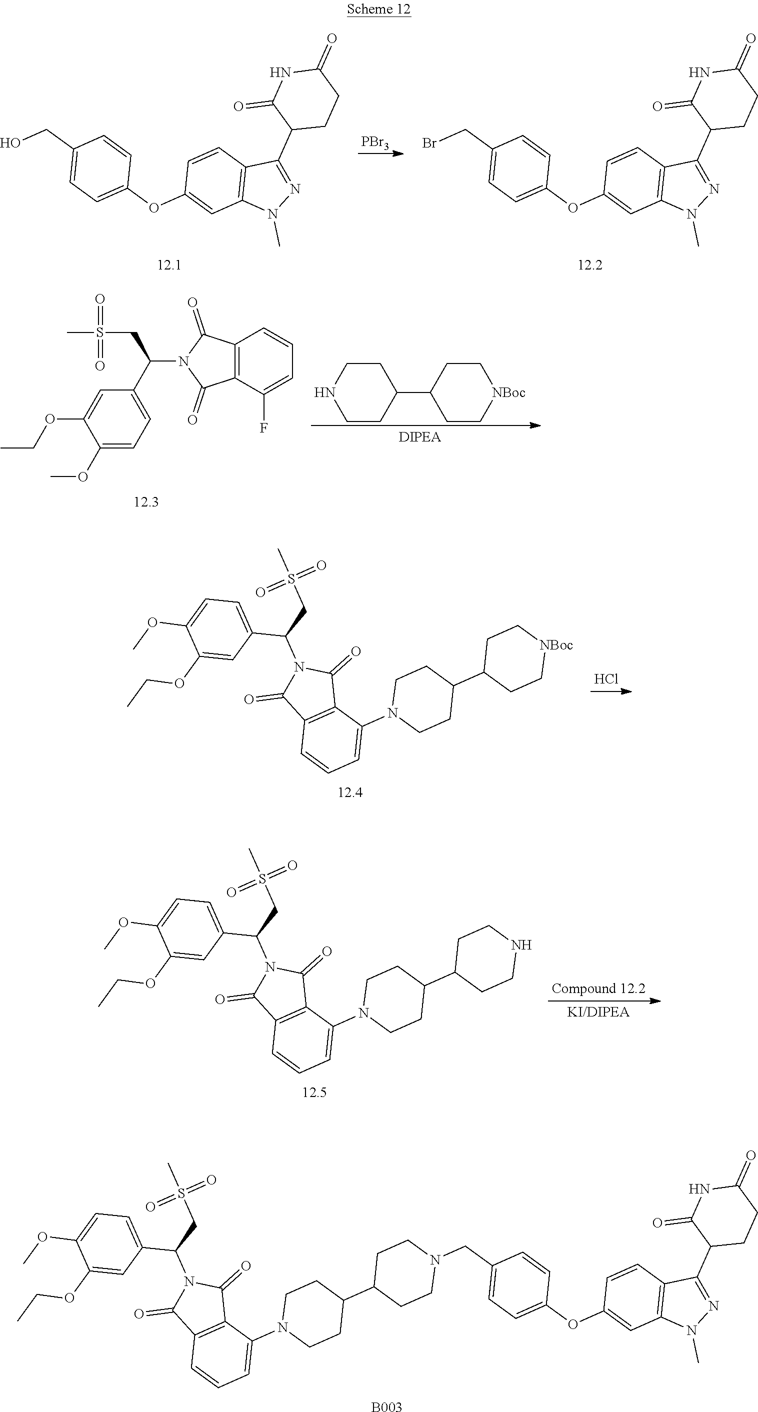

Preparation of 4-(1′-(4-((3-(2,6-dioxopiperidin-3-yl)-1-methyl-1H-indazol-6-yl)oxy)benzyl)-[4,4′-bipiperidin]-1-yl)-2-((S)-1-(3-ethoxy-4-methoxyphenyl)-2-(methyl-sulfonyl)ethyl)isoindoline-1,3-dione B003. To a solution of compound 12.2 (106 mg, 247 μmol) and compound 12.5 (150 mg, 248 μmol) in DMF (1 mL) were added DIPEA (96 mg, 742 μmol) and KI (41.1 mg, 248 μmol). After stirred at 80° C. for 12 h, the reaction mixture was filtered and purified by reverse-phase prep-HPLC to afford compound B003 (106 mg) in 47% yield. ¹H NMR (400 MHz, DMSO-d₆) δ 10.89 (s, 1H), 8.13 (s, 1H), 7.75 (d, J=8.8 Hz, 1H), 7.66-7.61 (m, 1H), 7.40 (dd, J=2.0, 6.0 Hz, 1H), 7.31-7.27 (m, 2H), 7.08-7.04 (m, 2H), 6.93 (s, 2H), 6.86 (dd, J=2.0, 8.8 Hz, 1H), 5.76 (dd, J=4.0, 10.4 Hz, 1H), 4.41-4.28 (m, 2H), 4.09 (dd, J=4.4, 14.0 Hz, 1H), 4.01 (q, J=7.2 Hz, 2H), 3.93 (s, 3H), 3.73 (s, 3H), 3.67 (s, 2H), 3.48-3.35 (m, 2H), 3.23 (s, 2H), 3.21-3.02 (m, 2H), 2.99 (s, 3H), 2.83-2.74 (m, 2H), 2.68-2.66 (m, 2H), 2.56 (s, 4H), 2.43-2.29 (m, 2H), 2.22-2.16 (m, 1H), 1.77 (d, J=10.8 Hz, 4H), 1.43-1.35 (m, 2H), 1.31 (t, J=7.2 Hz, 3H), 1.29-1.21 (m, 2H); MS (ESI) m/z: 917.7 [M+H]⁺.

### Example 13

### Preparation of 5-(1-((1-(4-((3-(2,6-dioxopiperidin-3-yl)-1-methyl-1H-indazol-6-yl)oxy)benzyl)-piperidin-4-yl)methyl)piperidin-4-yl)-2-((S)-1-(3-ethoxy-4-methoxyphenyl)-2-(methylsulfonyl)-ethyl)isoindoline-1,3-dione B004

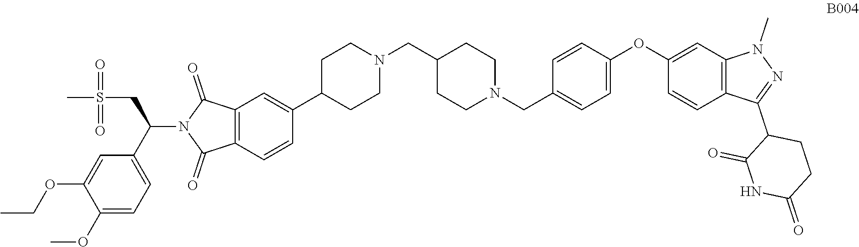 [SMILES: CCOc1cc([C@@H](CS(C)(=O)=O)N2C(=O)c3ccc(C4CCN(CC5CCN(Cc6ccc(Oc7ccc8c(C9CCC(=O)NC9=O)nn(C)c8c7)cc6)CC5)CC4)cc3C2=O)ccc1OC]

Compound B004 was prepared as shown in Scheme 13.

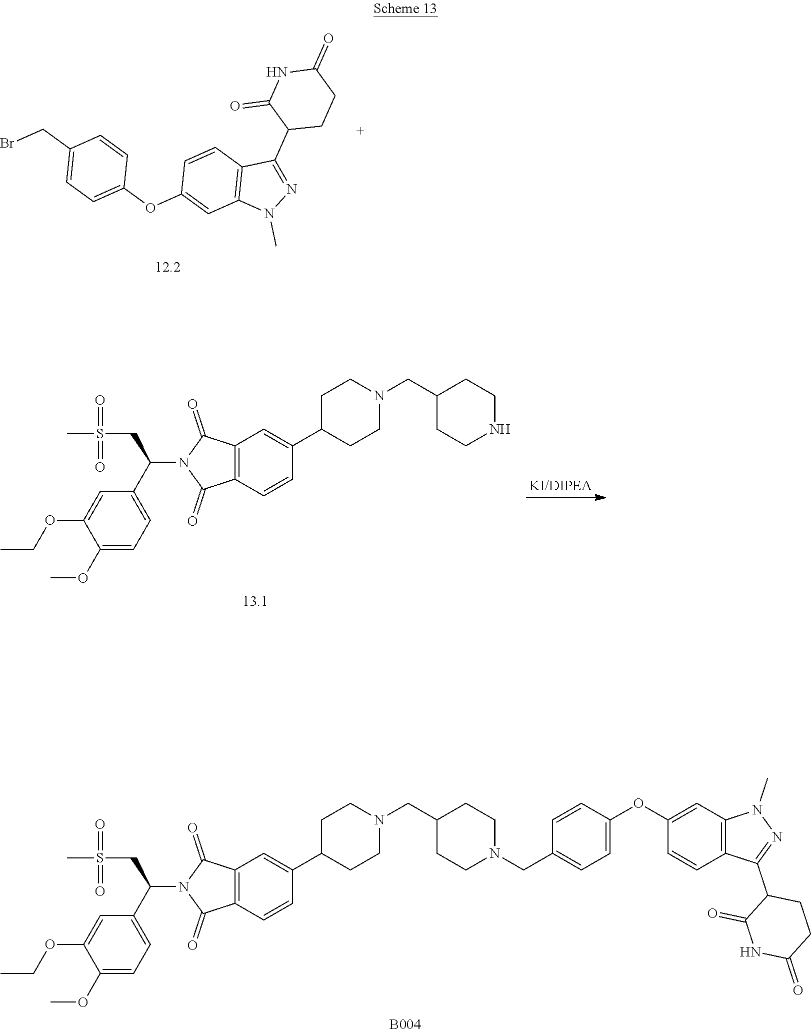 [SMILES: Cn1nc(C2CCC(=O)NC2=O)c2ccc(Oc3ccc(CBr)cc3)cc21 ; CCOc1cc([C@@H](CS(C)(=O)=O)N2C(=O)c3ccc(C4CCN(CC5CCNCC5)CC4)cc3C2=O)ccc1OC ; "13.1" ; CCOc1cc([C@@H](CS(C)(=O)=O)N2C(=O)c3ccc(C4CCN(CC5CCN(Cc6ccc(Oc7ccc8c(C9CCC(=O)NC9=O)nn(C)c8c7)cc6)CC5)CC4)cc3C2=O)ccc1OC ; "B004"]

To a solution of 3-[6-[4-(bromomethyl)phenoxy]-1-methylindazol-3-yl]-piperidine-2,6-dione 12.2 (100 mg, 233 μmol) and 2-[(1S)-1-(3-ethoxy-4-methoxyphenyl)-2-methylsulfonylethyl]-5-[1-(4-piperidylmethyl)-4-piperidyl]isoindoline-1,3-dione 13.1 (100 mg, 171 μmol) in DMF (1 mL) were added DIPEA (90.5 mg, 700 μmol) and KI (38.8 mg, 233 μmol). After stirred at 80° C. for 12 h, the reaction mixture was diluted with H₂O (3 mL). The resulting precipitates were collected by filtration and purified by reverse-phase prep-HPLC to afford compound B004 (25 mg) in 11% yield. ¹H NMR (400 MHz, DMSO-d₆) δ 10.90 (s, 1H), 8.21 (s, 1H), 7.71 (s, 4H), 7.29 (d, J=8.4 Hz, 2H), 7.21 (d, J=1.6 Hz, 1H), 7.07 (s, 1H), 7.02-6.87 (m, 4H), 6.84 (dd, J=1.6, 8.8 Hz, 1H), 5.77 (dd, J=4.4, 10.4 Hz, 1H), 4.40-4.30 (m, 2H), 4.11 (dd, J=4.2, 14.4 Hz, 1H), 4.00 (q, J=6.8 Hz, 2H), 3.91 (s, 3H), 3.72 (s, 3H), 3.00 (s, 3H), 2.93 (d, J=11.2 Hz, 2H), 2.81 (d, J=10.4 Hz, 2H), 2.74-2.56 (m, 4H), 2.41-2.31 (m, 2H), 2.22-2.12 (m, 3H), 2.01-1.88 (m, 4H), 1.82-1.62 (m, 6H), 1.56-1.44 (m, 1H), 1.31 (t, J=6.8 Hz, 3H), 1.17-1.03 (m, 2H); MS (ESI) mz: 931.8 [M+H]⁺.

### Example 14

### Preparation of 3-(6-(4-((1′-(2-((S)-1-(3-ethoxy-4-methoxyphenyl)-2-(methylsulfonyl)ethyl)-1-oxoisoindolin-5-yl)-[4,4′-bipiperidin]-1-yl)methyl)phenoxy)-1-methyl-1H-indazol-3-yl)-piperidine-2,6-dione B005

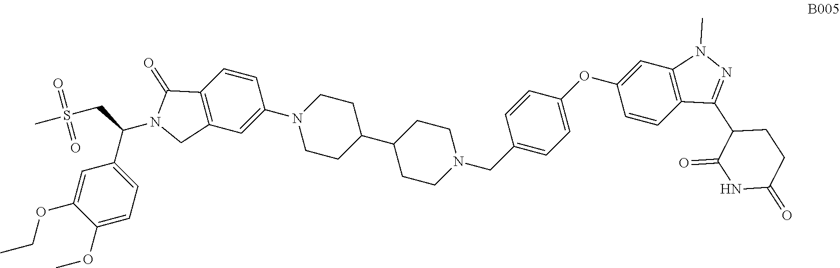 [SMILES: CCOc1cc([C@@H](CS(C)(=O)=O)N2Cc3cc(N4CCC(C5CCN(Cc6ccc(Oc7ccc8c(C9CCC(=O)NC9=O)nn(C)c8c7)cc6)CC5)CC4)ccc3C2=O)ccc1OC]

Compound B005 was prepared as shown in Scheme 14.

Preparation of tert-butyl 4-[1-[2-[(1S)-1-(3-ethoxy-4-methoxyphenyl)-2-methylsulfonylethyl]-1-oxoisoindolin-5-yl]-4-piperidyl]piperidine-1-carboxylate 14.2. To a solution of 5-bromo-2-[(1S)-1-(3-ethoxy-4-methoxyphenyl)-2-methylsulfonylethyl]isoindolin-1-one 14.1 (600 mg, 1.28 mmol) and tert-butyl 4-(4-piperidyl)piperidine-1-carboxylate (344 mg, 1.28 mmol) in dioxane (10 mL) were added RuPhos (59.8 mg, 128 μmol), Cs₂CO₃ (1.25 g, 3.84 mmol), and RuPhos Pd G3 (53.6 mg, 64.1 μmol) under N₂. After stirred under N₂ at 100° C. for 12 h, the reaction mixture was diluted with H₂O (15 mL) and extracted with EtOAc (20 mL×3). The combined organic layers were dried over anhydrous Na₂SO₄, filtered, and concentrated under reduced pressure to yield a crude product, which was purified by silica-gel column chromatography eluting with EtOAc/PE to afford compound 14.2 (720 mg) in 86% yield. MS (ESI) m/z: 656.6 [M+H]⁺.

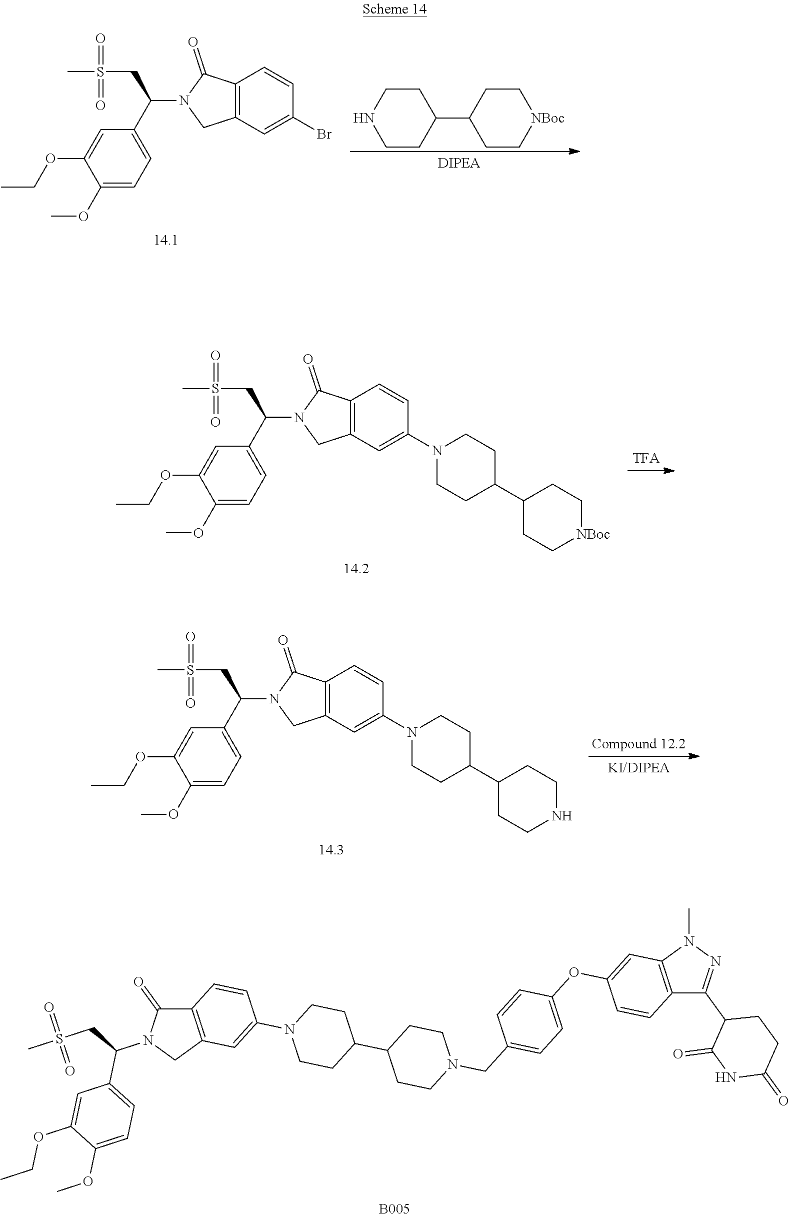

Preparation of 2-[(1S)-1-(3-ethoxy-4-methoxyphenyl)-2-methylsulfonylethyl]-5-[4-(4-piperidyl)-1-piperidyl]isoindolin-1-one 14.3. To a mixture of compound 14.2 (720 mg, 1.10 mmol) in DCM (2 mL) was added TFA (1.54 g, 13.46 mmol). After stirred at 20° C. for 2 h, the reaction mixture was concentrated under reduced pressure to afford compound 14.3 (500 mg), which was used directly in the next step without further purification.

Preparation of 3-(6-(4-((1′-(2-((S)-1-(3-ethoxy-4-methoxyphenyl)-2-(methyl-sulfonyl)ethyl)-1-oxoisoindolin-5-yl)-[4,4′-bipiperidin]-1-yl)methyl)phenoxy)-1-methyl-1H-indazol-3-yl)piperidine-2,6-dione B005. To a mixture of 3-[6-[4-(bromomethyl)phenoxy]-1-methyl-indazol-3-yl]piperidine-2,6-dione 12.2 (231 mg, 540 μmol) and compound 14.3 (200 mg, 360 μmol) in DMF (2 mL) were added KI (119 mg, 720 μmol), K₂CO₃ (249 mg, 1.8 mmol), and DIPEA (465 mg, 3.6 mmol). After stirred at 80° C. for 10 h, the reaction mixture was diluted with H₂O (10 mL) and extracted with DCM (15 mL×3). The combined organic layers were dried over anhydrous Na₂SO₄, filtered, and concentrated under reduced pressure to yield a crude product, which was purified by reverse-phase prep-HPLC to afford compound B005 (200 mg) in 62% yield. ¹H NMR (400 MHz, DMSO-d₆) δ 11.01-10.77 (m, 1H), 8.18 (s, 1H), 7.72 (d, J=8.4 Hz, 1H), 7.48-7.41 (m, 1H), 7.28 (d, J=8.4 Hz, 2H), 7.21 (s, 1H), 7.04-6.95 (m, 5H), 6.95-6.87 (m, 2H), 6.84 (d, J=8.0 Hz, 1H), 5.84 (dd, J=3.6, 10.0 Hz, 1H), 4.51-4.43 (m, 1H), 4.40-4.33 (m, 1H), 4.26-4.16 (m, 1H), 4.05-3.95 (m, 3H), 3.91 (s, 3H), 3.89-3.82 (m, 2H), 3.72 (s, 3H), 3.40 (s, 2H), 2.97 (s, 3H), 2.87-2.80 (m, 2H), 2.75-2.70 (m, 2H), 2.69-2.62 (m, 4H), 2.44-2.29 (m, 2H), 2.24-2.13 (m, 1H), 1.92-1.80 (m, 2H), 1.77-1.68 (m, 2H), 1.67-1.60 (m, 2H), 1.31 (t, J=7.2 Hz, 3H), 1.26-1.16 (m, 4H); MS (ESI) m/z: 904.0 [M+H]⁺.

### Example 15

### Preparation of 3-(6-(4-((1′-(2-((S)-1-(3-ethoxy-4-methoxyphenyl)-2-(methylsulfonyl)ethyl)-3-oxoisoindolin-5-yl)-[4,4′-bipiperidin]-1-yl)methyl)phenoxy)-1-methyl-1H-indazol-3-yl)-piperidine-2,6-dione B006

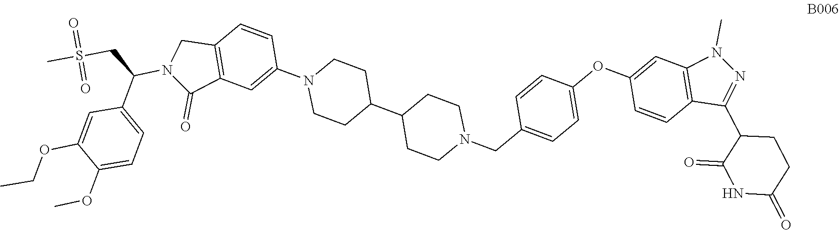 [SMILES: CCOc1cc([C@@H](CS(C)(=O)=O)N2Cc3ccc(N4CCC(C5CCN(Cc6ccc(Oc7ccc8c(C9CCC(=O)NC9=O)nn(C)c8c7)cc6)CC5)CC4)cc3C2=O)ccc1OC]

Compound B006 was prepared as shown in Scheme 15.

Preparation of 6-bromo-2-[(1S)-1-(3-ethoxy-4-methoxy-phenyl)-2-methylsulfonylethyl]isoindolin-1-one 15.2. To a solution of methyl 5-bromo-2-(bromomethyl)-benzoate (10 g, 32.5 mmol) and (1S)-1-(3-ethoxy-4-methoxyphenyl)-2-methylsulfonylethanamine 15.1 (9.76 g, 35.7 mmol) in ACN (100 mL) was added K₂CO₃ (13.5 g, 97.4 mmol). After stirred at 80° C. for 36 h, the reaction mixture was filtered and concentrated under reduced pressure to yield a crude product, which was purified by silica-gel column chromatography eluting with EtOAc/PE to afford compound 15.2 (12 g) in 79% yield. ¹H NMR (400 MHz, DMSO-d₆) δ 7.82 (s, 1H), 7.77 (dd, J=1.6, 8.0 Hz, 1H), 7.54 (d, J=8.4 Hz, 1H), 7.02 (s, 1H), 6.94 (s, 2H), 5.88 (dd, J=4.4, 10.4 Hz, 1H), 4.60 (d, J=17.6 Hz, 1H), 4.28 (dd, J=10.8, 14.4 Hz, 1H), 4.08 (d, J=17.6 Hz, 1H), 4.02 (t, J=7.2 Hz, 2H), 3.99-3.90 (m, 1H), 3.73 (s, 3H), 3 (s, 3H), 1.31 (t, J=7.2 Hz, 3H); MS (ESI) m/z: 468.1 [M+H]⁺.

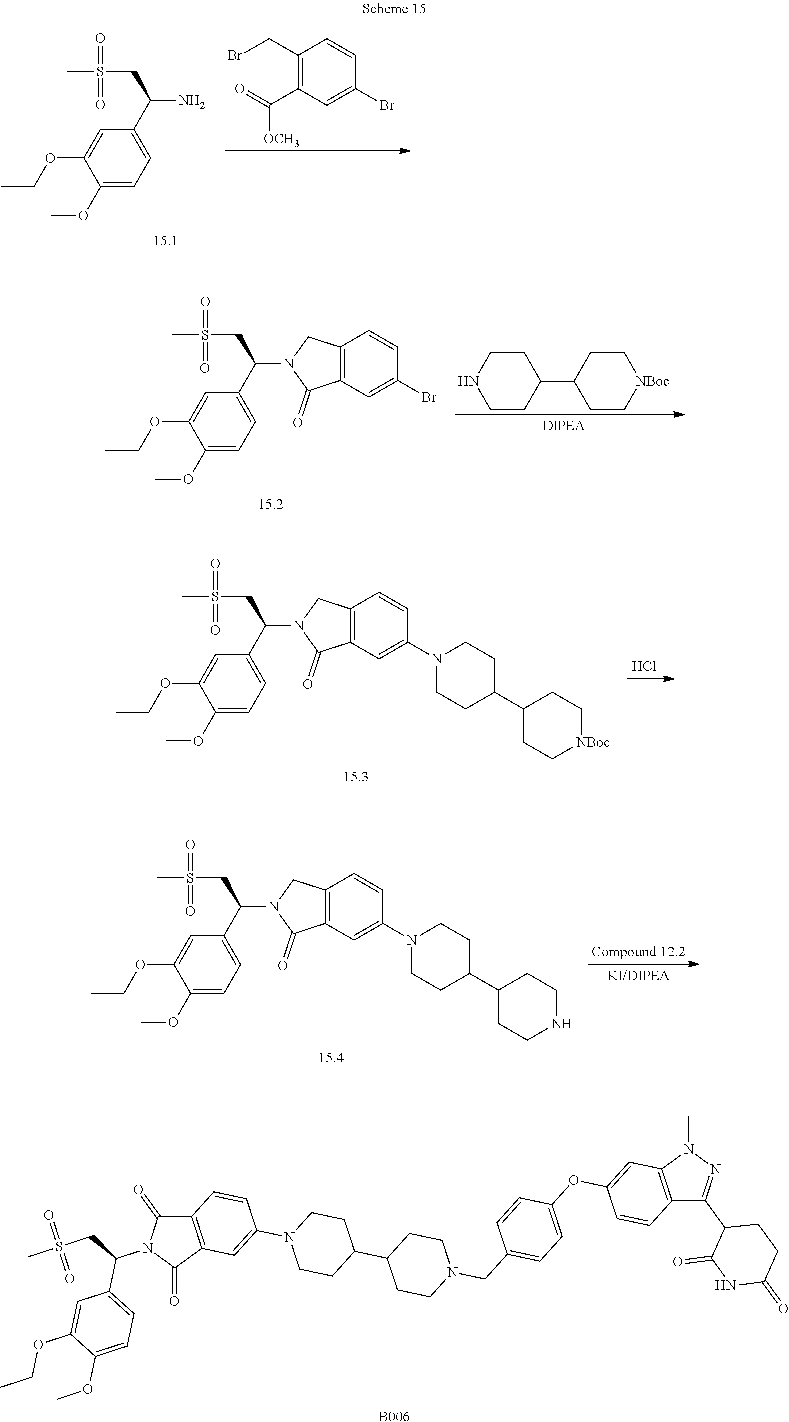

Preparation of tert-butyl 4-[1-[2-[(1S)-1-(3-ethoxy-4-methoxyphenyl)-2-methylsulfonylethyl]-3-oxoisoindolin-5-yl]-4-piperidyl]piperidine-1-carboxylate 15.3. To a solution of compound 15.2 (1 g, 2.14 mmol) and tert-butyl 4-(4-piperidyl)piperidine-1-carboxylate (573 mg, 2.14 mmol) in dioxane (15 mL) were added RuPhos (99.6 mg, 214 μmol), Cs₂CO₃ (2.09 g, 6.41 mmol), and RuPhos Pd G3 (89.3 mg, 107 μmol) under N₂. After stirred under N₂ at 100° C. for 12 h, the reaction mixture was diluted with H₂O (15 mL) and extracted with EtOAc (20 mL×3). The combined organic layers were dried over anhydrous Na₂SO₄, filtered, and concentrated under reduced pressure to yield a crude product, which was purified by silica-gel column chromatography eluting with EtOAc/PE to afford compound 15.3 (630 mg) in 45% yield. MS (ESI) mz: 656.4 [M+H]⁺.

Preparation of 2-[(1S)-1-(3-ethoxy-4-methoxyphenyl)-2-methylsulfonylethyl]-6-[4-(4-piperidyl)-1-piperidyl]isoindolin-1-one 15.4. To a mixture of compound 15.3 (630 mg, 961 μmol) in DCM (6 mL) was added 4M HCl in dioxane (3 mL). After stirred at 20° C. for 1 h, the reaction mixture was concentrated under reduced pressure to yield a crude product, which was triturated with EtOAc (20 mL) and DCM (10 mL) at 20° C. for 5 min to afford compound 15.4 (510 mg) in 94%. ¹H NMR (400 MHz, DMSO-d₆) δ 8.97 (d, J=10.0 Hz, 1H), 8.81 (d, J=9.6 Hz, 1H), 8.07-7.85 (m, 1H), 7.66 (d, J=2.8 Hz, 1H), 7.13-6.83 (m, 3H), 5.91 (dd, J=3.6, 10.4 Hz, 1H), 4.64 (d, J=17.6 Hz, 1H), 4.30 (dd, J=10.8, 14.0 Hz, 1H), 4.11 (d, J=17.6 Hz, 2H), 4.02 (t, J=6.8 Hz, 3H), 3.94 (dd, J=3.6, 14.4 Hz, 2H), 3.61 (d, J=10.4 Hz, 2H), 3.27 (d, J=12.0 Hz, 2H), 3.01 (s, 3H), 2.82 (d, J=11.2 Hz, 2H), 1.98 (s, 2H), 1.91-1.77 (m, J=12.6 Hz, 5H), 1.54-1.36 (m, J=3.6 Hz, 4H), 1.31 (t, J=6.8 Hz, 3H), 1.17 (t, J=7.2 Hz, 2H); MS (ESI) m/z: 556.7 [M+H]⁺.

Preparation of 3-(6-(4-((1′-(2-((S)-1-(3-ethoxy-4-methoxyphenyl)-2-(methyl-sulfonyl)ethyl)-3-oxoisoindolin-5-yl)-[4,4′-bipiperidin]-1-yl)methyl)phenoxy)-1-methyl-1H-indazol-3-yl)piperidine-2,6-dione B006. To a mixture of compound 12.2 (100 mg, 233 μmol) in DMF (2 mL) were added KI (77.5 mg, 467 μmol), DIPEA (90.5 mg, 700 μmol), and compound 15.4 (129.8 mg, 233 μmol). After stirred at 80° C. for 1 h, the reaction mixture was filtered and purified by reverse-phase prep-HPLC to afford compound B006 (23.1 mg) in 11% yield. ¹H NMR (400 MHz, DMSO-d₆) δ 10.88 (s, 1H), 7.72 (d, J=8.8 Hz, 1H), 7.34 (d, J=8.4 Hz, 1H), 7.29 (d, J=8.4 Hz, 2H), 7.23-7.15 (m, 2H), 7.09 (s, 1H), 6.99 (d, J=4.4 Hz, 2H), 6.97-6.94 (m, 1H), 6.94-6.89 (m, 2H), 6.84 (d, J=8.8 Hz, 1H), 5.88 (dd, J=4.4, 10.4 Hz, 1H), 4.48 (d, J=16.8 Hz, 1H), 4.36 (dd, J=5.2, 9.6 Hz, 1H), 4.24 (dd, J=10.8, 14.4 Hz, 1H), 4.06-3.92 (m, 4H), 3.92-3.87 (m, 3H), 3.75 (d, J=1.2 Hz, 1H), 3.73 (s, 3H), 3.40 (s, 2H), 3.28 (s, 2H), 2.98 (s, 3H), 2.84 (d, J=10.4 Hz, 2H), 2.68-2.61 (m, 4H), 2.35-2.31 (m, 1H), 2.23-2.15 (m, 1H), 1.86 (t, J=10.0 Hz, 2H), 1.80-1.71 (m, 2H), 1.65 (dd, J=1.6, 10.4 Hz, 2H), 1.31 (t, J=7.2 Hz, 3H), 1.26-1.14 (m, 4H), 1.12-0.99 (m, 1H); MS (ESI) m/z: 903.3 [M+H]⁺.

### Example 16

### Preparation of 4-(4-((1-(4-((3-(2,6-dioxopiperidin-3-yl)-1-methyl-1H-indazol-6-yl)oxy)benzyl)-piperidin-4-yl)methyl)piperazin-1-yl)-2-((S)-1-(3-ethoxy-4-methoxyphenyl)-2-(methylsulfonyl)-ethyl)isoindoline-1,3-dione B007

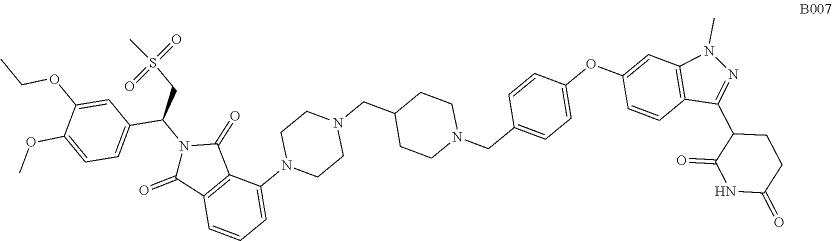 [SMILES: CCOc1cc([C@@H](CS(C)(=O)=O)N2C(=O)c3cccc(N4CCN(CC5CCN(Cc6ccc(Oc7ccc8c(C9CCC(=O)NC9=O)nn(C)c8c7)cc6)CC5)CC4)c3C2=O)ccc1OC]

Compound B007 was prepared as shown in Scheme 16.

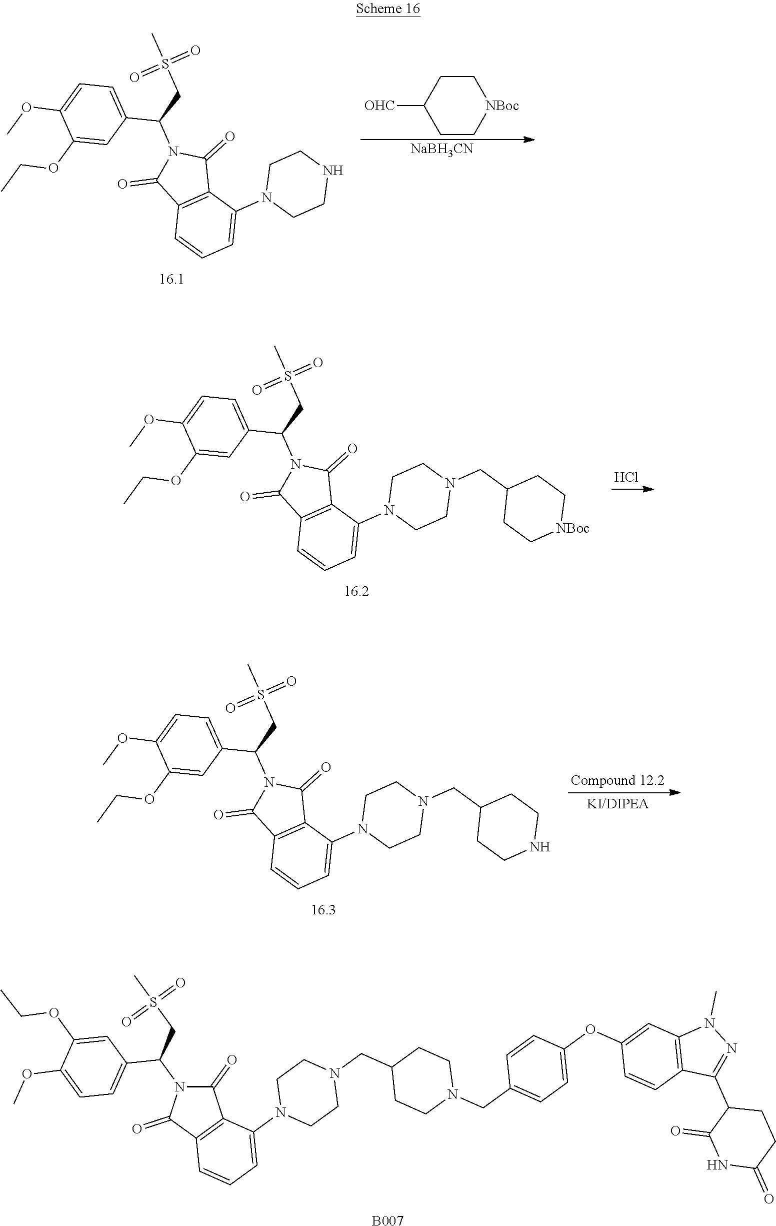

Preparation of tert-butyl 4-[[4-[2-[(1S)-1-(3-ethoxy-4-methoxyphenyl)-2-methylsulfonylethyl]-1,3-dioxoisoindolin-4-yl]piperazin-1-yl]methyl]piperidine-1-carboxylate 16.2. To a solution of 2-[(1S)-1-(3-ethoxy-4-methoxyphenyl)-2-methylsulfonylethyl]-4-piperazin-1-yl-isoindoline-1,3-dione 16.1 (1.00 g, 2.05 mmol) and tert-butyl 4-formylpiperidine-1-carboxylate (875 mg, 4.10 mmol) in MeOH (10 mL) were added NaBH₃CN (387 mg, 6.15 mmol) and NaOAc (337 mg, 4.10 mmol). After stirred at 20° C. for 12 h, the reaction mixture was diluted with H₂O (20 mL) and extracted with EtOAc (30 mL). The combined organic layers were washed with brine (30 mL), dried over anhydrous Na₂SO₄, filtered, and concentrated under reduced pressure to afford compound 16.2 (1.3 g) in 93% yield, which was used directly in the next step without further purification. MS (ESI) m/z: 685.4 [M+H]⁺.

Preparation of 2-[(1S)-1-(3-ethoxy-4-methoxyphenyl)-2-methylsulfonylethyl]-4-[4-(4-piperidylmethyl)piperazin-1-yl]isoindoline-1,3-dione 16.3. To a solution of compound 16.2 (1.3 g, 1.90 mmol) in DCM (5 mL) was added 4M HCl in dioxane (5 mL). After stirred at 20° C. for 1 h, the reaction mixture was concentrated under reduced pressure to afford compound 16.3 (1 g) in 90% yield, which was used directly in the next step without further purification.

Preparation of 4-(4-((1-(4-((3-(2,6-dioxopiperidin-3-yl)-1-methyl-1H-indazol-6-yl)oxy)benzyl)piperidin-4-yl)methyl)piperazin-1-yl)-2-((S)-1-(3-ethoxy-4-methoxyphenyl)-2-(methylsulfonyl)-ethyl)isoindoline-1,3-dione B007. To a solution of compound 12.2 (150 mg, 350 μmol) and compound 16.3 (120 mg, 205 μmol) in DMF (2 mL) were added DIPEA (136 mg, 1.05 mmol) and KI (58 mg, 350 μmol). After stirred at 80° C. for 12 h, the reaction mixture was diluted with H₂O (3 mL). The resulting precipitates were collected by filtration and purified by reverse-phase prep-HPLC to afford compound B007 (7.8 mg) in 2.4% yield. ¹H NMR (400 MHz, CDCl₃) δ 8.54-8.50 (m, 1H), 8.02-7.85 (m, 1H), 7.65 (d, J=9.2 Hz, 1H), 7.57-7.50 (m, 1H), 7.39-7.34 (m, 2H), 7.16-7.09 (m, 3H), 7.04-6.97 (m, 2H), 6.95-6.87 (m, 2H), 6.83 (d, J=8.0 Hz, 1H), 5.87 (dd, J=4.8, 9.6 Hz, 1H), 4.56-4.46 (m, 1H), 4.30 (dd, J=5.2, 7.2 Hz, 1H), 4.12 (q, J=7.2 Hz, 2H), 3.94 (s, 2H), 3.85 (s, 3H), 3.82 (d, J=5.2 Hz, 1H), 3.79-3.74 (m, 2H), 3.33 (s, 4H), 3.21-3.11 (m, 2H), 3.05-3.01 (m, 1H), 2.84-2.79 (m, 3H), 2.76-2.69 (m, 1H), 2.66 (d, J=4.4 Hz, 4H), 2.57-2.50 (m, 1H), 2.47-2.36 (m, 2H), 2.32 (d, J=7.2 Hz, 2H), 2.24 (d, J=6.4 Hz, 2H), 1.89-1.82 (m, 2H), 1.68-1.52 (m, 2H), 1.47 (t, J=7.2 Hz, 3H); MS (ESI) m/z: 932.5 [M+H]⁺.

### Example 17

### Preparation of (3R)-3-(6-(1′-(4-((3-(2,6-dioxopiperidin-3-yl)-1-methyl-1H-indazol-6-yl)oxy)-benzyl)-[4,4′-bipiperidin]-1-yl)-1-oxoisoindolin-2-yl)-3-(3-ethoxy-4-methoxyphenyl)-propanenitrile C011

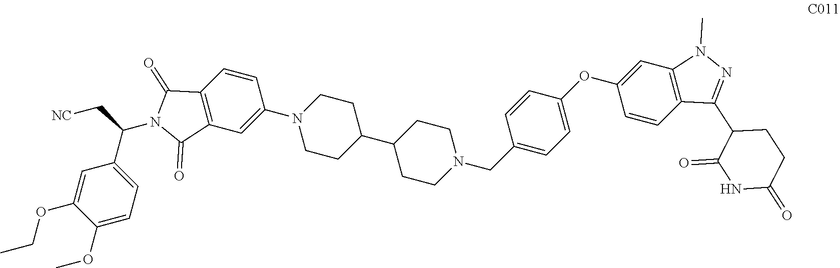 [SMILES: CCOc1cc([C@@H](CC#N)N2C(=O)c3ccc(N4CCC(C5CCN(Cc6ccc(Oc7ccc8c(C9CCC(=O)NC9=O)nn(C)c8c7)cc6)CC5)CC4)cc3C2=O)ccc1OC]

Compound C011 was prepared as shown in Scheme 17.

Preparation of (R,E)-N-(3-ethoxy-4-methoxybenzylidene)-2-methylpropane-2-sulfinamide 17.2. To a solution of 3-ethoxy-4-methoxybenzaldehyde 17.1 (5 g, 27.8 mmol) and (R)-2-methylpropane-2-sulfinamide (3.7 g, 30.5 mmol) in THF (50 mL) was added Ti(OEt)₄ (12.7 g, 55.5 mmol). After stirred at 60° C. for 6 h, the reaction mixture was diluted with aqueous NaCl (100 mL), filtered, and extracted with EtOAc (100 mL). The combined organic layers were dried over anhydrous Na₂SO₄, filtered, and concentrated under reduced pressure to afford compound 17.2 (6.8 g) in 86% yield, which was used directly in the next step without further purification. MS (ESI) m/z: 284.2 [M+H]⁺.

Preparation of (R)—N-[(1R)-2-cyano-1-(3-ethoxy-4-methoxy-phenyl)ethyl]-2-methylpropane-2-sulfinamide 17.3. To ACN (145 mg, 3.53 mmol) was added 2.5M n-BuLi in THF (1.41 mL) dropwise at −78° C. After the mixture was stirred at −78° C. for 1 h under N₂, compound 17.2 (500 mg, 1.76 mmol) in THF (10 mL) was added dropwise at −78° C. The resulting mixture was stirred at −78° C. for 1 h, and then was diluted with 1M HCl and extracted with EtOAc (10 mL×3). The combined organic layers were washed with brine (5 mL), dried over anhydrous Na₂SO₄, filtered, and concentrated under reduced pressure to yield a crude product, which was purified by silica-gel column chromatography eluting with MeOH/DCM to afford compound 17.3. MS (ESI) m/z: 325.2 [M+H]⁺.

Preparation of (3R)-3-amino-3-(3-ethoxy-4-methoxy-phenyl)propanenitrile 17.4. To a solution of compound 17.3 (500 mg, 1.54 mmol) in DCM (5 mL) was added 4M HCl in dioxane (2.31 mL) at 0° C. After stirred at 20° C. for 2 h, the reaction mixture was concentrated under reduced pressure to afford compound 17.4 (600 mg) as a HCl salt, which was used directly in the next step without further purification. MS (EST) m/z: 441.2 [M+H]⁺.

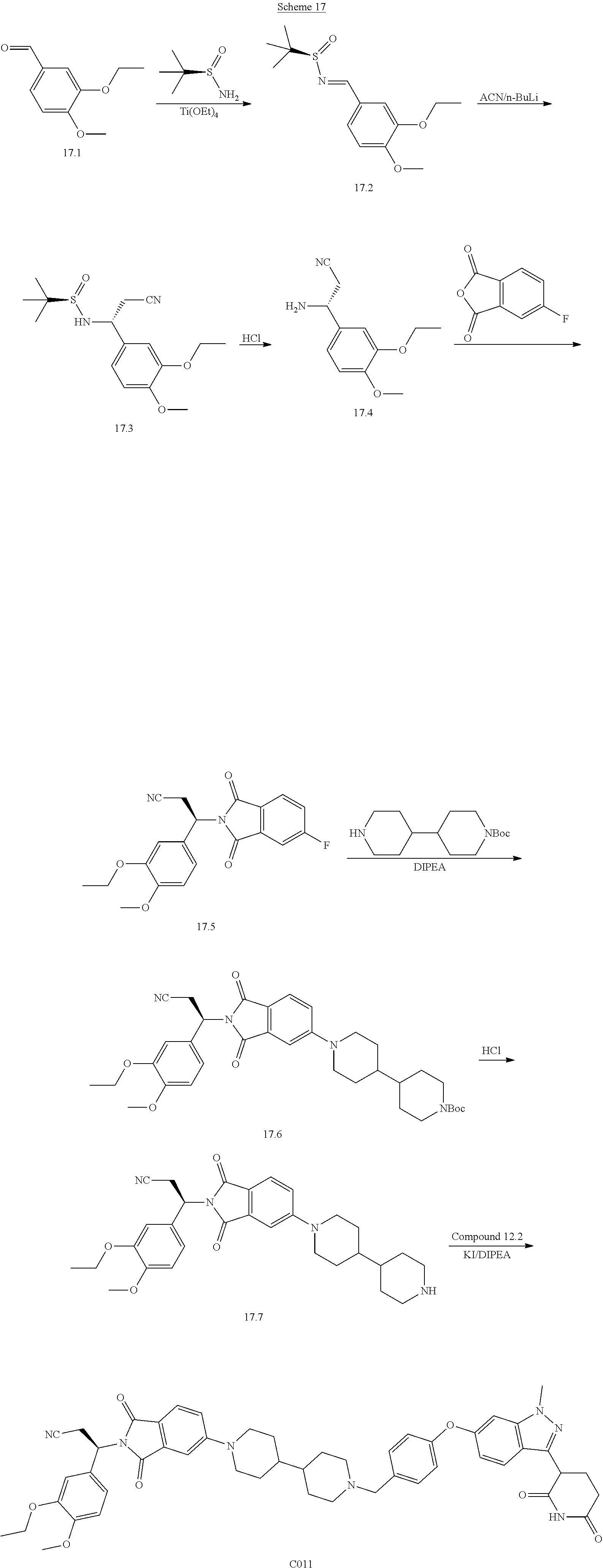

Preparation of (3R)-3-(3-ethoxy-4-methoxy-phenyl)-3-(5-fluoro-1,3-dioxo-isoindolin-2-yl)propanenitrile 17.5. To a solution of compound 17.4 (600 mg, 2.34 mmol) in AcOH (8 mL) were added NaOAc (383 mg, 4.67 mmol) and 5-fluoroisobenzofuran-1,3-dione (427 mg, 2.57 mmol). After stirred at 110° C. for 12 h, the reaction mixture was diluted with H₂O (20 mL) and extracted with EtOAc (10 mL×3). The combined organic layers were washed with brine (20 mL), dried over anhydrous Na₂SO₄, filtered, and concentrated under reduced pressure to afford a crude product, which was purified by silica-gel column chromatography eluting with EtOAc/PE to afford compound 17.5 (270 mg) in 31% yield. MS (ESI) m/z: 369.2 [M+H]⁺.

Preparation of tert-butyl 4-[1-[2-[(1R)-2-cyano-1-(3-ethoxy-4-methoxyphenyl)-ethyl]-1,3-dioxo-isoindolin-5-yl]-4-piperidyl]piperidine-1-carboxylate 17.6. To a solution of compound 17.5 (270 mg, 733 μmol) in DMSO (5 mL) were added DIPEA (189 mg, 1.47 mmol) and tert-butyl 4-(4-piperidyl)piperidine-1-carboxylate (216 mg, 806 μmol). After stirred at 120° C. for 5 h, the reaction mixture was diluted with H₂O (20 mL) and extracted with EtOAc (10 mL×3). The combined organic layers were washed with brine (10 mL), dried over anhydrous Na₂SO₄, filtered, and concentrated under reduced pressure to yield a crude product, which was purified by silica-gel column chromatography eluting with EtOAc/PE to afford compound 17.6 (420 mg) in 93% yield. MS (ESI) m/z: 617.6 [M+H]⁺.

Preparation of (3R)-3-[1,3-dioxo-5-[4-(4-piperidyl)-1-piperidyl]isoindolin-2-yl]-3-(3-ethoxy-4-methoxyphenyl) propanenitrile 17.7. To a solution of compound 17.6 (200 mg, 324 μmol) in DCM (2 mL) was added 4M HCl in dioxane (2 mL) at 0° C. After stirred at 20° C. for 1 h, the reaction mixture was concentrated under reduced pressure to afford compound 17.7 (300 mg) as a HCl salt, which was used directly in the next step without further purification. MS (ESI) m/z: 517.3 [M+H]⁺.

Preparation of (3R)-3-[5-[4-[1-[[4-[3-(2,6-dioxo-3-piperidyl)-1-methylindazol-6-yl]oxyphenyl]methyl]-4-piperidyl]-1-piperidyl]-1,3-dioxoisoindolin-2-yl]-3-(3-ethoxy-4-methoxyphenyl)propanenitrile C011. To a solution of 3-[6-[4-(bromomethyl)phenoxy]-1-methyl-indazol-3-yl]piperidine-2,6-dione 12.2 (200 mg, 467 μmol) and compound 17.7 (258 mg, 467 μmol) in DMF (2 mL) were added DIPEA (181 mg, 1.40 mmol) and KI (77.5 mg, 467 μmol). After stirred at 80° C. for 1 h, the reaction mixture was diluted with H₂O (20 mL) and extracted with EtOAc (10 mL×3). The combined organic layers were washed with brine (20 mL), dried over anhydrous Na₂SO₄, filtered, and concentrated under reduced pressure to yield a crude product, which was purified by reverse-phase prep-HPLC to afford compound C011 (147 mg) in 36% yield. ¹H NMR (400 MHz, DMSO-d₆) δ 10.90 (s, 1H), 8.16 (s, 1H), 7.72 (d, J=8.8 Hz, 1H), 7.63 (d, J=8.8 Hz, 1H), 7.33-7.25 (m, 3H), 7.21 (d, J=1.6 Hz, 2H), 7.02 (s, 1H), 6.98 (d, J=8.4 Hz, 2H), 6.93-6.86 (m, 2H), 6.83 (dd, J=1.6, 8.8 Hz, 1H), 5.52 (dd, J=6.4, 10.4 Hz, 1H), 4.36 (dd, J=5.2, 9.6 Hz, 1H), 4.06 (d, J=13.6 Hz, 2H), 3.97 (q, J=6.8 Hz, 2H), 3.91 (s, 3H), 3.72 (s, 3H), 3.71-3.65 (m, 1H), 3.51 (dd, J=6.4, 16.8 Hz, 2H), 3.41 (s, 2H), 2.93-2.80 (m, 4H), 2.65 (td, J=5.2, 14.8 Hz, 2H), 2.40-2.30 (m, 1H), 2.23-2.14 (m, 1H), 1.86 (t, J=10.8 Hz, 2H), 1.75 (d, J=11.6 Hz, 2H), 1.64 (d, J=12.4 Hz, 2H), 1.30 (t, J=7.2 Hz, 3H), 1.19 (q, J=12.4 Hz, 4H), 1.10-1 (m, 1H); MS (ESI) m/z: 864.5 [M+H]⁺.

The following compounds were prepared similarly according to the synthetic procedures or methodologies exemplified herein.

5-(6-(4-(4-(2,6-Dioxopiperidin-3-yl)phenyl)-[1,4′-bipiperidin]-1′-yl)hexyl)-2-((S)-1-(3-ethoxy-4-methoxyphenyl)-2-(methylsulfonyl)ethyl)isoindoline-1,3-dione A002.

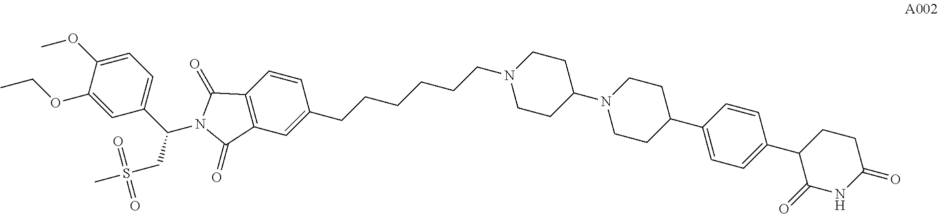 [SMILES: CCOc1cc([C@@H](CS(C)(=O)=O)N2C(=O)c3ccc(CCCCCCN4CCC(N5CCC(c6ccc(C7CCC(=O)NC7=O)cc6)CC5)CC4)cc3C2=O)ccc1OC]

3-(4-(1′-(5-(2-((S)-1-(3-Ethoxy-4-methoxyphenyl)-2-(methylsulfonyl)ethyl)-3-oxoisoindolin-5-yl)pentyl)-[1,4′-bipiperidin]-4-yl)phenyl)piperidine-2,6-dione A003.

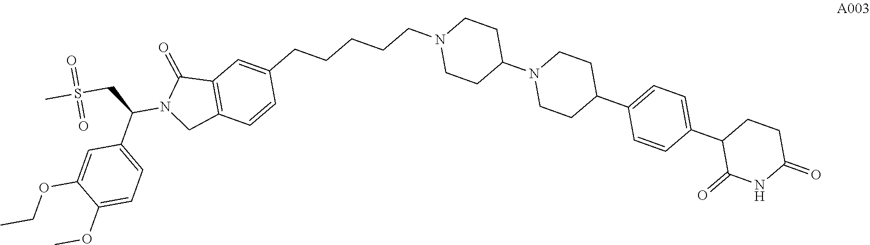 [SMILES: CCOc1cc([C@@H](CS(C)(=O)=O)N2Cc3ccc(CCCCCN4CCC(N5CCC(c6ccc(C7CCC(=O)NC7=O)cc6)CC5)CC4)cc3C2=O)ccc1OC]

5-(5-(4-(3-(2,6-Dioxopiperidin-3-yl)phenyl)-[1,4′-bipiperidin]-1′-yl)pentyl)-2-((S)-1-(3-ethoxy-4-methoxyphenyl)-2-(methylsulfonyl)ethyl)isoindoline-1,3-dione A005.

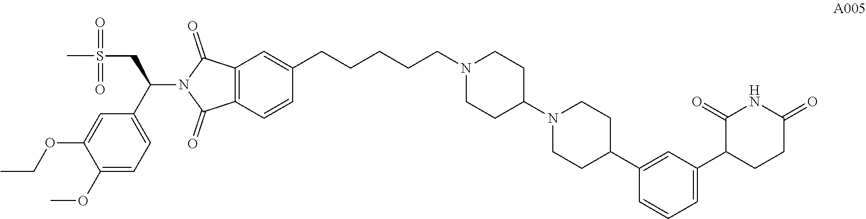 [SMILES: CCOc1cc([C@@H](CS(C)(=O)=O)N2C(=O)c3ccc(CCCCCN4CCC(N5CCC(c6cccc(C7CCC(=O)NC7=O)c6)CC5)CC4)cc3C2=O)ccc1OC]

5-(4-(1-((1-(4-(2,6-Dioxopiperidin-3-yl)phenyl)piperidin-4-yl)methyl)piperidin-4-yl)butyl)-2-((S)-1-(3-ethoxy-4-methoxyphenyl)-2-(methylsulfonyl)ethyl)isoindoline-1,3-dione A007.

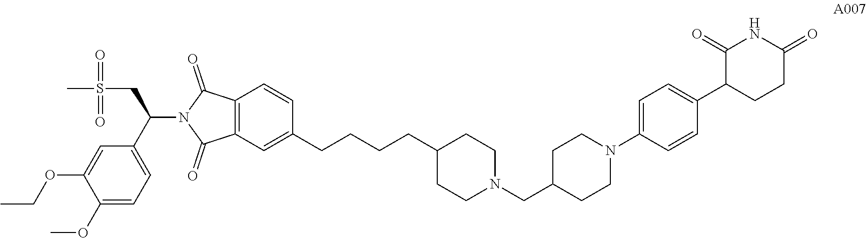 [SMILES: CCOc1cc([C@@H](CS(C)(=O)=O)N2C(=O)c3ccc(CCCCC4CCN(CC5CCN(c6ccc(C7CCC(=O)NC7=O)cc6)CC5)CC4)cc3C2=O)ccc1OC]

4-(3-(4-(4-(2,6-Dioxopiperidin-3-yl)phenyl)-[1,4′:1′,4″-terpiperidin]-1″-yl)-propyl)-2-((S)-1-(3-ethoxy-4-methoxyphenyl)-2-(methylsulfonyl)ethyl)isoindoline-1,3-dione A008.

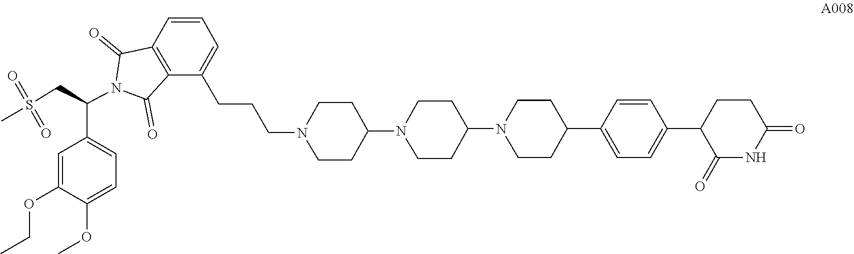 [SMILES: CCOc1cc([C@@H](CS(C)(=O)=O)N2C(=O)c3cccc(CCCN4CCC(N5CCC(N6CCC(c7ccc(C8CCC(=O)NC8=O)cc7)CC6)CC5)CC4)c3C2=O)ccc1OC]

5-(3-(4-(4-(2,6-Dioxopiperidin-3-yl)phenyl)-[1,4′:1′,4″-terpiperidin]-1″-yl)-propyl)-2-((S)-1-(3-ethoxy-4-methoxyphenyl)-2-(methylsulfonyl)ethyl)isoindoline-1,3-dione A009. ¹H NMR (400 MHz, DMSO-d₆) δ 10.80 (s, 1H), 8.21 (d, J=4.8 Hz, 2H), 7.78 (d, J=8.0 Hz, 1H), 7.73 (s, 1H), 7.68 (d, J=8.4 Hz, 1H), 7.24-7.17 (m, 2H), 7.16-7.11 (m, 2H), 7.09 (d, J=1.6 Hz, 1H), 7.00-6.90 (m, 2H), 5.77 (dd, J=4.4, 10.4 Hz, 1H), 4.38-4.32 (m, 1H), 4.14-4.09 (m, 1H), 4.04-3.99 (m, 2H), 3.82-3.78 (m, 1H), 3.73 (s, 3H), 3.06 (s, 2H), 2.99 (s, 3H), 2.96-2.87 (m, 4H), 2.76 (t, J=7.2 Hz, 2H), 2.68-2.60 (m, 1H), 2.46 (d, J=4.8 Hz, 2H), 2.35-2.23 (m, 6H), 2.20 (s, 3H), 2.06-1.98 (m, 1H), 1.92-1.83 (m, 2H), 1.80-1.72 (m, 6H), 1.72-1.59 (m, 4H), 1.49-1.36 (m, 4H), 1.32 (t, J=7.2 Hz, 3H).

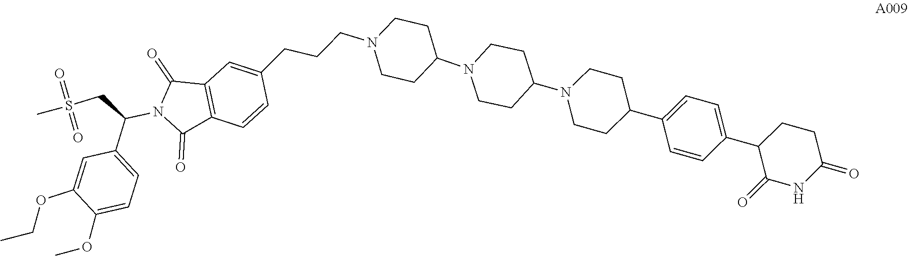 [SMILES: CCOc1cc([C@@H](CS(C)(=O)=O)N2C(=O)c3ccc(CCCN4CCC(N5CCC(N6CCC(c7ccc(C8CCC(=O)NC8=O)cc7)CC6)CC5)CC4)cc3C2=O)ccc1OC]

4-(2-(4-(4-(2,6-Dioxopiperidin-3-yl)phenyl)-[1,4′:1′,4″-terpiperidin]-1″-yl)ethyl)-2-((S)-1-(3-ethoxy-4-methoxyphenyl)-2-(methylsulfonyl)ethyl)isoindoline-1,3-dione A010.

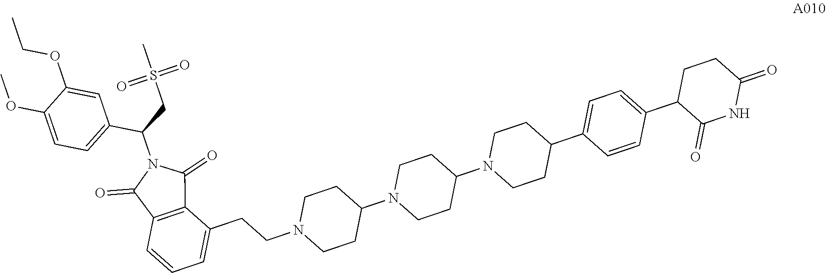 [SMILES: CCOc1cc([C@@H](CS(C)(=O)=O)N2C(=O)c3cccc(CCN4CCC(N5CCC(N6CCC(c7ccc(C8CCC(=O)NC8=O)cc7)CC6)CC5)CC4)c3C2=O)ccc1OC]

5-(2-(4-(4-(2,6-Dioxopiperidin-3-yl)phenyl)-[1,4′:1′,4″-terpiperidin]-1″-yl)ethyl)-2-((S)-1-(3-ethoxy-4-methoxyphenyl)-2-(methylsulfonyl)ethyl)isoindoline-1,3-dione A011.

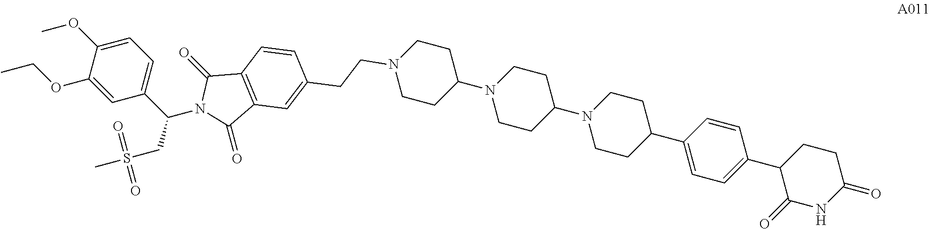 [SMILES: CCOc1cc([C@@H](CS(C)(=O)=O)N2C(=O)c3ccc(CCN4CCC(N5CCC(N6CCC(c7ccc(C8CCC(=O)NC8=O)cc7)CC6)CC5)CC4)cc3C2=O)ccc1OC]

4-((1′-(3-(4-(4-(2,6-Dioxopiperidin-3-yl)phenyl)piperidin-1-yl)propyl)-[1,4′-bipiperidin]-4-yl)amino)-2-((S)-1-(3-ethoxy-4-methoxyphenyl)-2-(methylsulfonyl)ethyl)-isoindoline-1,3-dione A016. ¹H NMR (400 MHz, CDCl₃) δ 8.46 (s, 1H), 8.13 (s, 1H), 7.45 (t, J=7.6 Hz, 1H), 7.24 (s, 2H), 7.19-7.15 (m, 2H), 7.13-7.09 (m, 2H), 7.07 (d, J=7.2 Hz, 1H), 6.83 (dd, J=5.3, 8.4 Hz, 2H), 6.29 (d, J=7.6 Hz, 1H), 5.83 (dd, J=4.4, 9.6 Hz, 1H), 4.53 (dd, J=10.0, 14.0 Hz, 1H), 4.12 (q, J=7.2 Hz, 2H), 3.85 (s, 3H), 3.79 (t, J=4.8 Hz, 1H), 3.76 (d, J=5.2 Hz, 1H), 3.51 (d, J=12.0 Hz, 3H), 3.27 (d, J=12.0 Hz, 2H), 3.07 (d, J=11.2 Hz, 3H), 2.83 (s, 3H), 2.78-2.71 (m, 4H), 2.71-2.56 (m, 7H), 2.43-2.33 (m, 2H), 2.31-2.24 (m, 2H), 2.23-2.12 (m, 6H), 2.07-2.01 (m, 2H), 2.00-1.92 (m, 4H), 1.84-1.71 (m, 2H), 1.47 (t, J=7.2 Hz, 3H); MS (ESI) m/z: 897.9 [M+H]⁺.

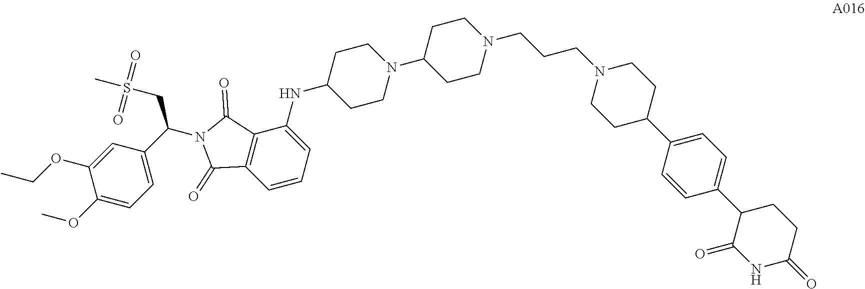 [SMILES: CCOc1cc([C@@H](CS(C)(=O)=O)N2C(=O)c3cccc(NC4CCN(C5CCN(CCCN6CCC(c7ccc(C8CCC(=O)NC8=O)cc7)CC6)CC5)CC4)c3C2=O)ccc1OC]

5-((1′-(3-(4-(4-(2,6-Dioxopiperidin-3-yl)phenyl)piperidin-1-yl)propyl)-[1,4′-bipiperidin]-4-yl)amino)-2-((S)-1-(3-ethoxy-4-methoxyphenyl)-2-(methylsulfonyl)ethyl)-isoindoline-1,3-dione A017.

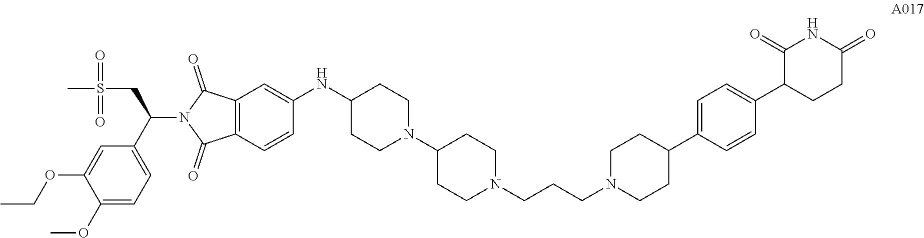 [SMILES: CCOc1cc([C@@H](CS(C)(=O)=O)N2C(=O)c3ccc(NC4CCN(C5CCN(CCCN6CCC(c7ccc(C8CCC(=O)NC8=O)cc7)CC6)CC5)CC4)cc3C2=O)ccc1OC]

4-((1′-(2-(4-(4-(2,6-Dioxopiperidin-3-yl)phenyl)piperidin-1-yl)ethyl)-[1,4′-bipiperidin]-4-yl)amino)-2-((S)-1-(3-ethoxy-4-methoxyphenyl)-2-(methylsulfonyl)ethyl)-isoindoline-1,3-dione A018.

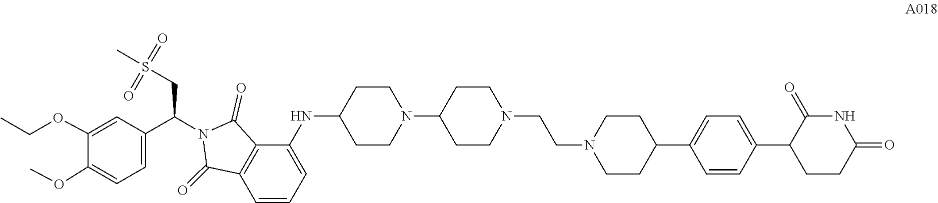 [SMILES: CCOc1cc([C@@H](CS(C)(=O)=O)N2C(=O)c3cccc(NC4CCN(C5CCN(CCN6CCC(c7ccc(C8CCC(=O)NC8=O)cc7)CC6)CC5)CC4)c3C2=O)ccc1OC]

5-((1′-(2-(4-(4-(2,6-Dioxopiperidin-3-yl)phenyl)piperidin-1-yl)ethyl)-[1,4′-bipiperidin]-4-yl)amino)-2-((S)-1-(3-ethoxy-4-methoxyphenyl)-2-(methylsulfonyl)ethyl)-isoindoline-1,3-dione A019.

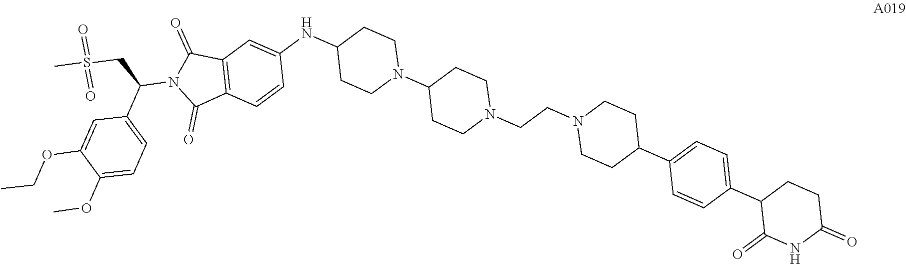 [SMILES: CCOc1cc([C@@H](CS(C)(=O)=O)N2C(=O)c3ccc(NC4CCN(C5CCN(CCN6CCC(c7ccc(C8CCC(=O)NC8=O)cc7)CC6)CC5)CC4)cc3C2=O)ccc1OC]

4-(4-(1-(3-(4-(4-(2,6-Dioxopiperidin-3-yl)phenyl)piperidin-1-yl)propyl)piperidin-4-yl)piperazin-1-yl)-2-((S)-1-(3-ethoxy-4-methoxyphenyl)-2-(methylsulfonyl)ethyl)isoindoline-1,3-dione A020.

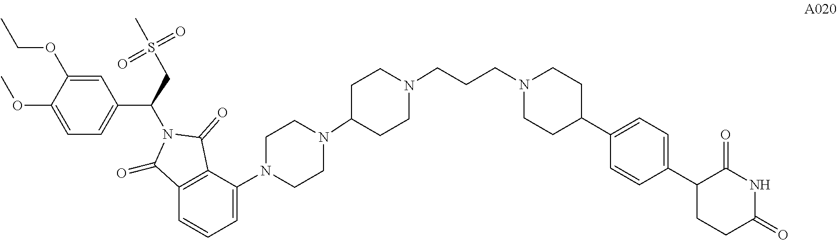 [SMILES: CCOc1cc([C@@H](CS(C)(=O)=O)N2C(=O)c3cccc(N4CCN(C5CCN(CCCN6CCC(c7ccc(C8CCC(=O)NC8=O)cc7)CC6)CC5)CC4)c3C2=O)ccc1OC]

5-(4-(1-(3-(4-(4-(2,6-Dioxopiperidin-3-yl)phenyl)piperidin-1-yl)propyl)piperidin-4-yl)piperazin-1-yl)-2-((S)-1-(3-ethoxy-4-methoxyphenyl)-2-(methylsulfonyl)ethyl)isoindoline-1,3-dione A021.

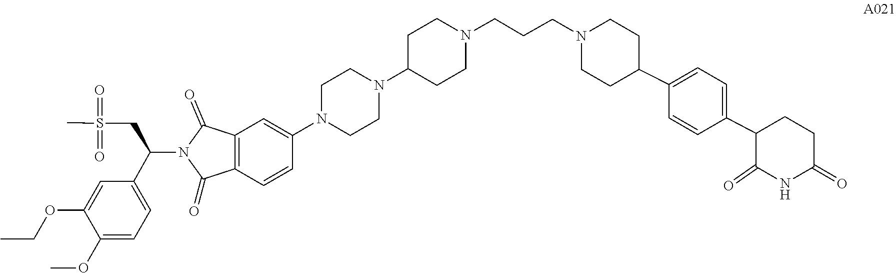 [SMILES: CCOc1cc([C@@H](CS(C)(=O)=O)N2C(=O)c3ccc(N4CCN(C5CCN(CCCN6CCC(c7ccc(C8CCC(=O)NC8=O)cc7)CC6)CC5)CC4)cc3C2=O)ccc1OC]

4-(4-(1-(2-(4-(4-(2,6-Dioxopiperidin-3-yl)phenyl)piperidin-1-yl)ethyl)piperidin-4-yl)-piperazin-1-yl)-2-((S)-1-(3-ethoxy-4-methoxyphenyl)-2-(methylsulfonyl)ethyl)isoindoline-1,3-dione A022. ¹H NMR (400 MHz, CDCl₃) δ 8.47 (s, 1H), 8.13 (s, 1H), 7.59-7.53 (m, 1H), 7.38 (d, J=7.2 Hz, 1H), 7.26-7.22 (m, 2H), 7.18-7.15 (m, 2H), 7.14-7.10 (m, 3H), 6.83 (d, J=8.8 Hz, 1H), 5.87 (dd, J=4.8, 10.0 Hz, 1H), 4.53 (dd, J=10.0, 14.4 Hz, 1H), 4.11 (q, J=6.8 Hz, 2H), 3.85 (s, 3H), 3.80 (d, J=5.2 Hz, 1H), 3.77-3.75 (m, 1H), 3.49-3.34 (m, 5H), 3.30-3.20 (m, 2H), 2.96-2.88 (m, 7H), 2.82 (s, 3H), 2.70-2.56 (m, 5H), 2.55-2.45 (m, 2H), 2.40-2.30 (m, 2H), 2.31-2.18 (m, 2H), 2.10-1.97 (m, 4H), 1.96-1.89 (m, 2H), 1.87-1.76 (m, 2H), 1.47 (t, J=7.2 Hz, 3H); MS (ESI) m/z: 869.6 [M+H]⁺.

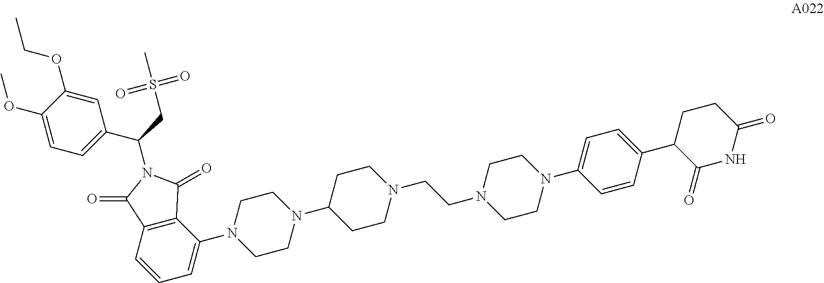 [SMILES: CCOc1cc([C@@H](CS(C)(=O)=O)N2C(=O)c3cccc(N4CCN(C5CCN(CCN6CCN(c7ccc(C8CCC(=O)NC8=O)cc7)CC6)CC5)CC4)c3C2=O)ccc1OC]

5-(4-(1-(2-(4-(4-(2,6-Dioxopiperidin-3-yl)phenyl)piperidin-1-yl)ethyl)piperidin-4-yl)piperazin-1-yl)-2-((S)-1-(3-ethoxy-4-methoxyphenyl)-2-(methylsulfonyl)ethyl)isoindoline-1,3-dione A023.

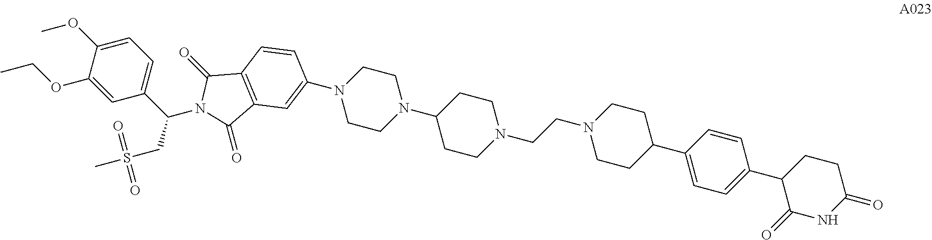 [SMILES: CCOc1cc([C@@H](CS(C)(=O)=O)N2C(=O)c3ccc(N4CCN(C5CCN(CCN6CCC(c7ccc(C8CCC(=O)NC8=O)cc7)CC6)CC5)CC4)cc3C2=O)ccc1OC]

5-(4-(1-((1-(4-(2,6-Dioxopiperidin-3-yl)phenyl)piperidin-4-yl)methyl)piperidin-4-yl)piperazin-1-yl)-2-((S)-1-(3-ethoxy-4-methoxyphenyl)-2-(methylsulfonyl)ethyl)isoindoline-1,3-dione A024.

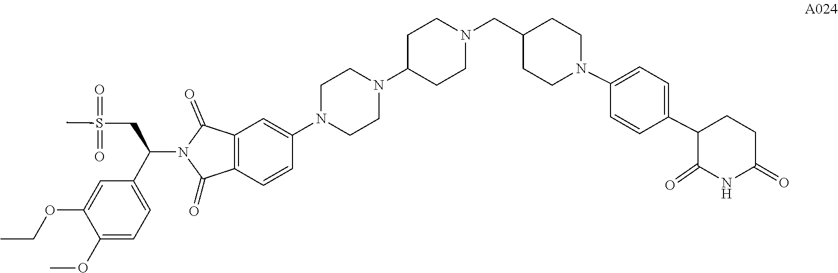 [SMILES: CCOc1cc([C@@H](CS(C)(=O)=O)N2C(=O)c3ccc(N4CCN(C5CCN(CC6CCN(c7ccc(C8CCC(=O)NC8=O)cc7)CC6)CC5)CC4)cc3C2=O)ccc1OC]

5-(4-(1-(2-(1-(4-(2,6-Dioxopiperidin-3-yl)phenyl)piperidin-4-yl)ethyl)azetidin-3-yl)piperazin-1-yl)-2-((S)-1-(3-ethoxy-4-methoxyphenyl)-2-(methylsulfonyl)ethyl)isoindoline-1,3-dione A025.

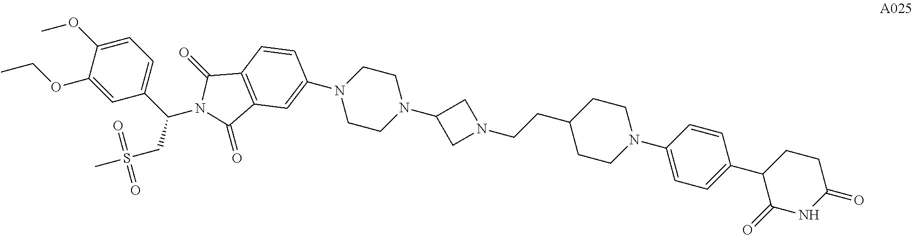 [SMILES: CCOc1cc([C@@H](CS(C)(=O)=O)N2C(=O)c3ccc(N4CCN(C5CN(CCC6CCN(c7ccc(C8CCC(=O)NC8=O)cc7)CC6)C5)CC4)cc3C2=O)ccc1OC]

4-(5-(4-(3-(2,6-Dioxopiperidin-3-yl)phenyl)-[1,4′-bipiperidin]-1′-yl)pentyl)-2-((S)-1-(3-ethoxy-4-methoxyphenyl)-2-(methylsulfonyl)ethyl)isoindoline-1,3-dione A027.

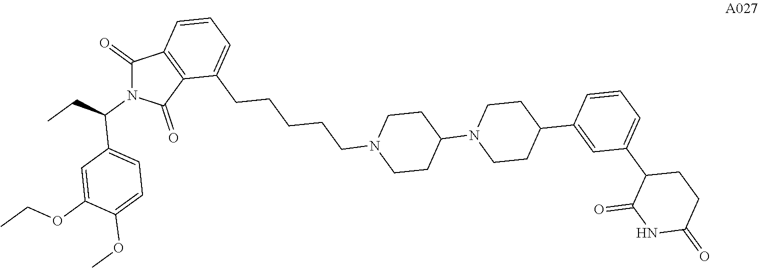 [SMILES: CCOc1cc([C@@H](CC)N2C(=O)c3cccc(CCCCCN4CCC(N5CCC(c6cccc(C7CCC(=O)NC7=O)c6)CC5)CC4)c3C2=O)ccc1OC]

5-(1-((1′-(4-(2,6-Dioxopiperidin-3-yl)phenyl)-[1,4′-bipiperidin]-4-yl)methyl)-piperidin-4-yl)-2-((S)-1-(3-ethoxy-4-methoxyphenyl)-2-(methylsulfonyl)ethyl)isoindoline-1,3-dione A028. ¹H NMR (400 MHz, DMSO-d₆) δ 10.76 (s, 1H), 8.28 (s, 1H), 7.90-7.64 (m, 3H), 7.14-6.79 (m, 7H), 5.78 (dd, J=3.2, 6.0 Hz, 1H), 4.34 (s, 1H), 4.13 (s, 1H), 4.03-3.98 (m, 2H), 3.72 (s, 3H), 3.00 (s, 3H), 2.95-2.87 (m, 3H), 2.80-2.57 (m, 8H), 2.27 (s, 3H), 2.16 (s, 3H), 2.06-1.92 (m, 4H), 1.88-1.81 (m, 2H), 1.80-1.63 (m, 6H), 1.60-1.47 (m, 3H), 1.31 (s, 3H), 1.23-1.10 (m, 2H); MS (ESI) m/z: 854.5 [M+H]⁺.

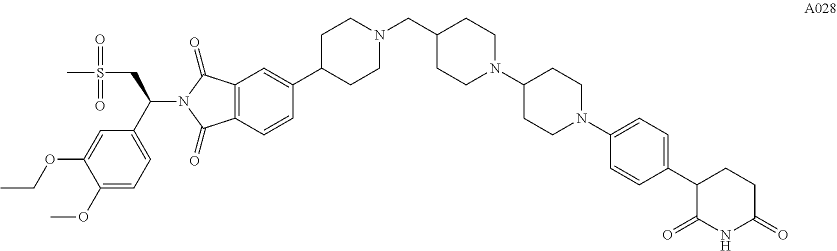 [SMILES: CCOc1cc([C@@H](CS(C)(=O)=O)N2C(=O)c3ccc(C4CCN(CC5CCN(C6CCN(c7ccc(C8CCC(=O)NC8=O)cc7)CC6)CC5)CC4)cc3C2=O)ccc1OC]

5-(1′-(2-(4-(4-(2,6-Dioxopiperidin-3-yl)phenyl)piperidin-1-yl)ethyl)-[4,4′-bipiperidin]-1-yl)-2-((S)-1-(3-ethoxy-4-methoxyphenyl)-2-(methylsulfonyl)ethyl)isoindoline-1,3-dione A029.

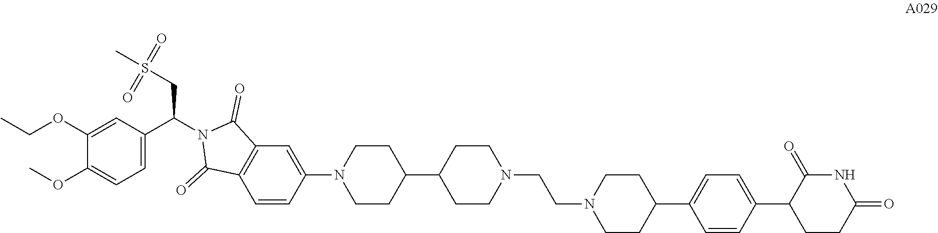 [SMILES: CCOc1cc([C@@H](CS(C)(=O)=O)N2C(=O)c3ccc(N4CCC(C5CCN(CCN6CCC(c7ccc(C8CCC(=O)NC8=O)cc7)CC6)CC5)CC4)cc3C2=O)ccc1OC]

5-(1′-(4-(2,6-Dioxopiperidin-3-yl)phenyl)-[4,4′-bipiperidin]-1-yl)-2-((S)-1-(3-ethoxy-4-methoxyphenyl)-2-(methylsulfonyl)ethyl)isoindoline-1,3-dione A030.

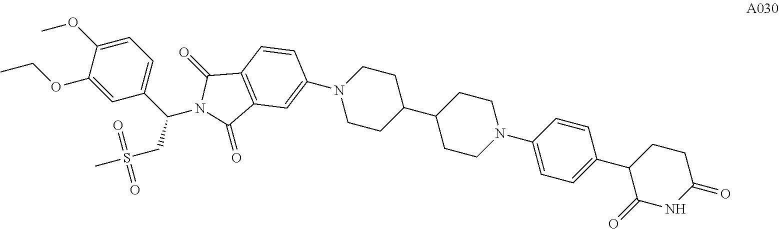 [SMILES: CCOc1cc([C@@H](CS(C)(=O)=O)N2C(=O)c3ccc(N4CCC(C5CCN(c6ccc(C7CCC(=O)NC7=O)cc6)CC5)CC4)cc3C2=O)ccc1OC]

4-(4-(1-(2-(4-(4-(2,6-Dioxopiperidin-3-yl)phenyl)piperazin-1-yl)ethyl)piperidin-4-yl)piperazin-1-yl)-2-((S)-1-(3-ethoxy-4-methoxyphenyl)-2-(methylsulfonyl)ethyl)isoindoline-1,3-dione A032.

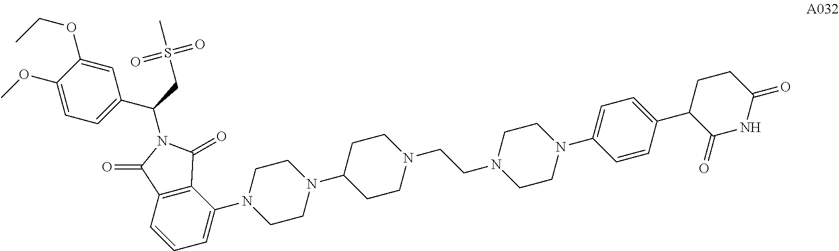 [SMILES: CCOc1cc([C@@H](CS(C)(=O)=O)N2C(=O)c3cccc(N4CCN(C5CCN(CCN6CCN(c7ccc(C8CCC(=O)NC8=O)cc7)CC6)CC5)CC4)c3C2=O)ccc1OC]

5-(4-(1-(2-(4-(4-(2,6-Dioxopiperidin-3-yl)phenyl)piperazin-1-yl)ethyl)piperidin-4-yl)piperazin-1-yl)-2-((S)-1-(3-ethoxy-4-methoxyphenyl)-2-(methylsulfonyl)ethyl)isoindoline-1,3-dione A033.

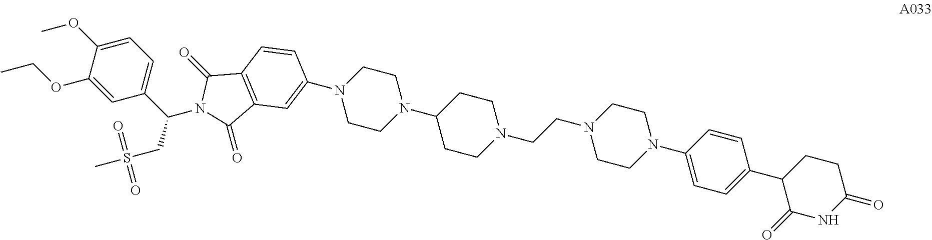 [SMILES: CCOc1cc([C@@H](CS(C)(=O)=O)N2C(=O)c3ccc(N4CCN(C5CCN(CCN6CCN(c7ccc(C8CCC(=O)NC8=O)cc7)CC6)CC5)CC4)cc3C2=O)ccc1OC]

5-(4-(2-(1′-(4-(2,6-Dioxopiperidin-3-yl)phenyl)-[4,4′-bipiperidin]-1-yl)ethyl)-piperazin-1-yl)-2-((S)-1-(3-ethoxy-4-methoxyphenyl)-2-(methylsulfonyl)ethyl)isoindoline-1,3-dione A034.

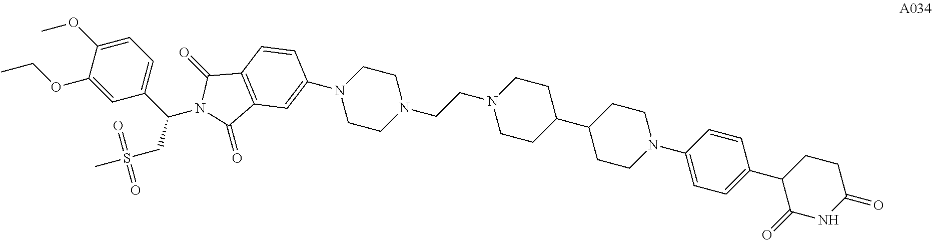 [SMILES: CCOc1cc([C@@H](CS(C)(=O)=O)N2C(=O)c3ccc(N4CCN(CCN5CCC(C6CCN(c7ccc(C8CCC(=O)NC8=O)cc7)CC6)CC5)CC4)cc3C2=O)ccc1OC]

4-(4-(2-(1′-(4-(2,6-Dioxopiperidin-3-yl)phenyl)-[4,4′-bipiperidin]-1-yl)ethyl)-piperazin-1-yl)-2-((S)-1-(3-ethoxy-4-methoxyphenyl)-2-(methylsulfonyl)ethyl)isoindoline-1,3-dione A035.

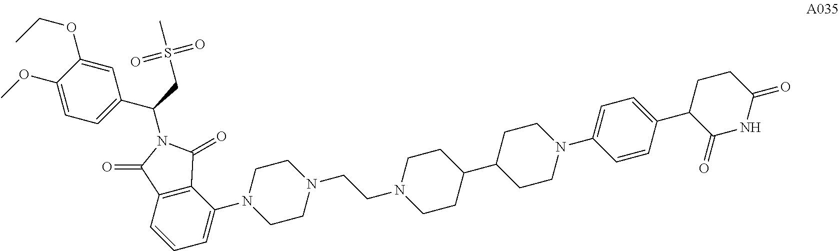 [SMILES: CCOc1cc([C@@H](CS(C)(=O)=O)N2C(=O)c3cccc(N4CCN(CCN5CCC(C6CCN(c7ccc(C8CCC(=O)NC8=O)cc7)CC6)CC5)CC4)c3C2=O)ccc1OC]

5-(4-(4-(4-(3-(2,6-Dioxopiperidin-3-yl)phenyl)but-3-yn-1-yl)piperazin-1-yl)-piperidin-1-yl)-2-((S)-1-(3-ethoxy-4-methoxyphenyl)-2-(methylsulfonyl)ethyl)isoindoline-1,3-dione A038.

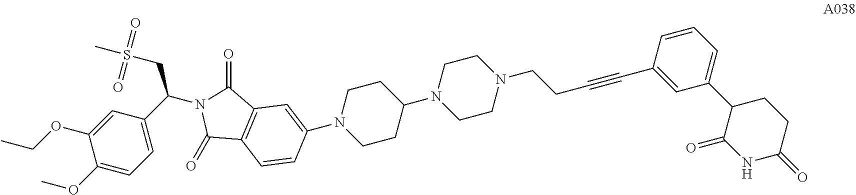 [SMILES: CCOc1cc([C@@H](CS(C)(=O)=O)N2C(=O)c3ccc(N4CCC(N5CCN(CCC#Cc6cccc(C7CCC(=O)NC7=O)c6)CC5)CC4)cc3C2=O)ccc1OC]

5-(4-((3-(2,6-Dioxopiperidin-3-yl)phenyl)ethynyl)-[1,4′-bipiperidin]-1′-yl)-2-((S)-1-(3-ethoxy-4-methoxyphenyl)-2-(methylsulfonyl)ethyl)isoindoline-1,3-dione A039.

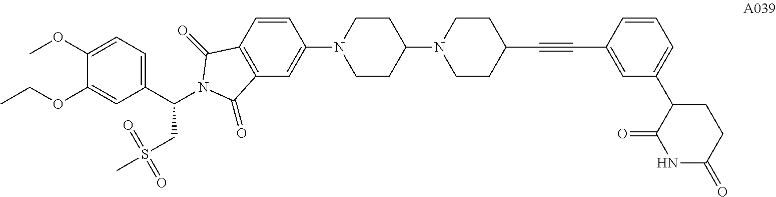 [SMILES: CCOc1cc([C@@H](CS(C)(=O)=O)N2C(=O)c3ccc(N4CCC(N5CCC(C#Cc6cccc(C7CCC(=O)NC7=O)c6)CC5)CC4)cc3C2=O)ccc1OC]

5-(4-(4-(4-(4-(2,6-Dioxopiperidin-3-yl)phenyl)but-3-yn-1-yl)piperazin-1-yl)-piperidin-1-yl)-2-((S)-1-(3-ethoxy-4-methoxyphenyl)-2-(methylsulfonyl)ethyl)isoindoline-1,3-dione A040.

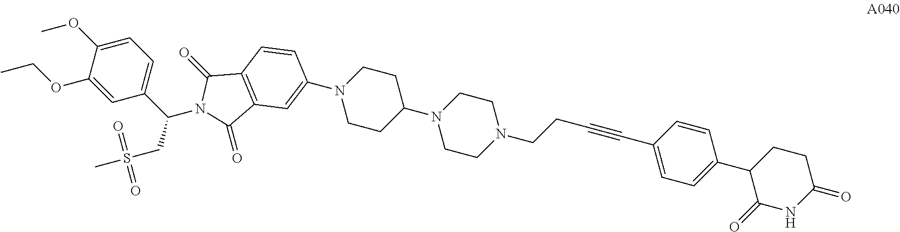 [SMILES: CCOc1cc([C@@H](CS(C)(=O)=O)N2C(=O)c3ccc(N4CCC(N5CCN(CCC#Cc6ccc(C7CCC(=O)NC7=O)cc6)CC5)CC4)cc3C2=O)ccc1OC]

5-(4-((4-((4-(4-(2,6-Dioxopiperidin-3-yl)phenyl)piperidin-1-yl)methyl)piperidin-1-yl)methyl)piperidin-1-yl)-2-((S)-1-(3-ethoxy-4-methoxyphenyl)-2-(methylsulfonyl)ethyl)-isoindoline-1,3-dione A042.

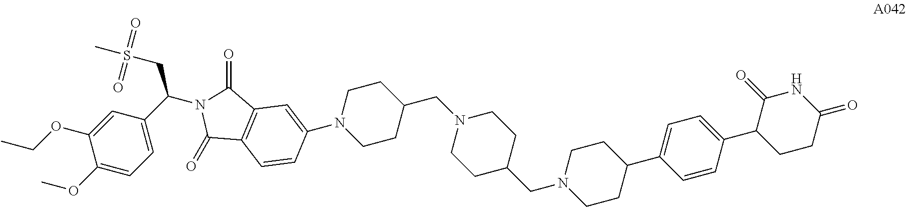 [SMILES: CCOc1cc([C@@H](CS(C)(=O)=O)N2C(=O)c3ccc(N4CCC(CN5CCC(CN6CCC(c7ccc(C8CCC(=O)NC8=O)cc7)CC6)CC5)CC4)cc3C2=O)ccc1OC]

5-((1-((1-(2-(4-(4-(2,6-Dioxopiperidin-3-yl)phenyl)piperidin-1-yl)ethyl)piperidin-4-yl)methyl)piperidin-4-yl)amino)-2-((S)-1-(3-ethoxy-4-methoxyphenyl)-2-(methylsulfonyl)-ethyl)isoindoline-1,3-dione A043.

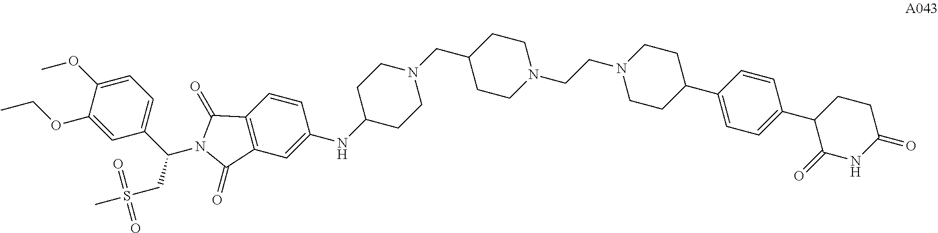 [SMILES: CCOc1cc([C@@H](CS(C)(=O)=O)N2C(=O)c3ccc(NC4CCN(CC5CCN(CCN6CCC(c7ccc(C8CCC(=O)NC8=O)cc7)CC6)CC5)CC4)cc3C2=O)ccc1OC]

5-(4-((1′-(4-(2,6-Dioxopiperidin-3-yl)phenyl)-[4,4′-bipiperidin]-1-yl)methyl)-piperidin-1-yl)-2-((S)-1-(3-ethoxy-4-methoxyphenyl)-2-(methylsulfonyl)ethyl)isoindoline-1,3-dione A044.

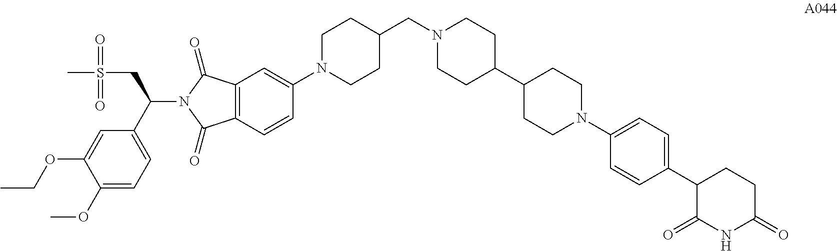 [SMILES: CCOc1cc([C@@H](CS(C)(=O)=O)N2C(=O)c3ccc(N4CCC(CN5CCC(C6CCN(c7ccc(C8CCC(=O)NC8=O)cc7)CC6)CC5)CC4)cc3C2=O)ccc1OC]

5-(4-((1-(4-(2,6-Dioxopiperidin-3-yl)phenyl)piperidin-4-yl)methyl)piperazin-1-yl)-2-((S)-1-(3-ethoxy-4-methoxyphenyl)-2-(methylsulfonyl)ethyl)isoindoline-1,3-dione A046.

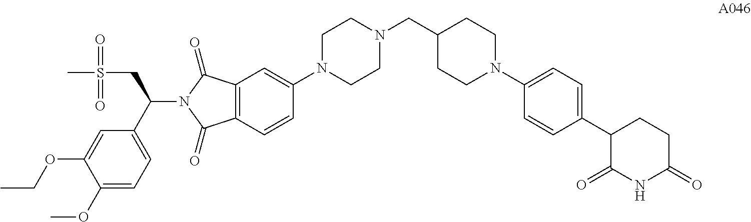 [SMILES: CCOc1cc([C@@H](CS(C)(=O)=O)N2C(=O)c3ccc(N4CCN(CC5CCN(c6ccc(C7CCC(=O)NC7=O)cc6)CC5)CC4)cc3C2=O)ccc1OC]

4-(4-(1-(4-(4-(2,6-Dioxopiperidin-3-yl)phenyl)cyclohexyl)piperidin-4-yl)-piperazin-1-yl)-2-((S)-1-(3-ethoxy-4-methoxyphenyl)-2-(methylsulfonyl)ethyl)isoindoline-1,3-dione A047.

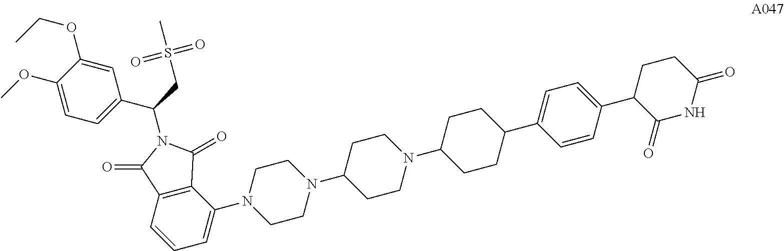 [SMILES: CCOc1cc([C@@H](CS(C)(=O)=O)N2C(=O)c3cccc(N4CCN(C5CCN(C6CCC(c7ccc(C8CCC(=O)NC8=O)cc7)CC6)CC5)CC4)c3C2=O)ccc1OC]

4-(4-(1-(3-(2,6-Dioxopiperidin-3-yl)phenethyl)piperidin-4-yl)piperazin-1-yl)-2-((S)-1-(3-ethoxy-4-methoxyphenyl)-2-(methylsulfonyl)ethyl)isoindoline-1,3-dione A050.

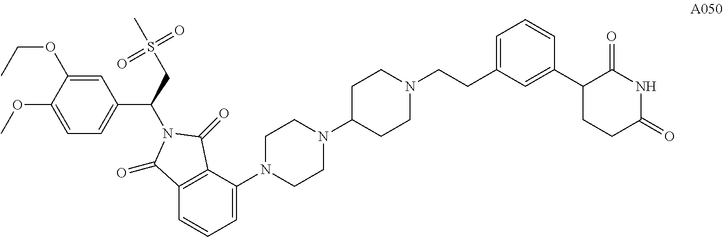 [SMILES: CCOc1cc([C@@H](CS(C)(=O)=O)N2C(=O)c3cccc(N4CCN(C5CCN(CCc6cccc(C7CCC(=O)NC7=O)c6)CC5)CC4)c3C2=O)ccc1OC]

5-(4-(1-(3-(2,6-Dioxopiperidin-3-yl)phenethyl)piperidin-4-yl)piperazin-1-yl)-2-((S)-1-(3-ethoxy-4-methoxyphenyl)-2-(methylsulfonyl)ethyl)isoindoline-1,3-dione A051.

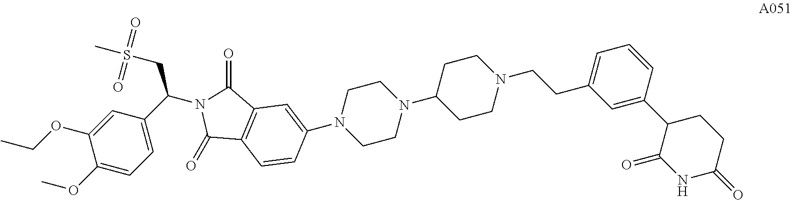 [SMILES: CCOc1cc([C@@H](CS(C)(=O)=O)N2C(=O)c3ccc(N4CCN(C5CCN(CCc6cccc(C7CCC(=O)NC7=O)c6)CC5)CC4)cc3C2=O)ccc1OC]

4-(4-((1-(2-(4-(4-(2,6-Dioxopiperidin-3-yl)phenyl)piperidin-1-yl)ethyl)piperidin-4-yl)methyl)piperazin-1-yl)-2-((S)-1-(3-ethoxy-4-methoxyphenyl)-2-(methylsulfonyl)ethyl)-isoindoline-1,3-dione A052.

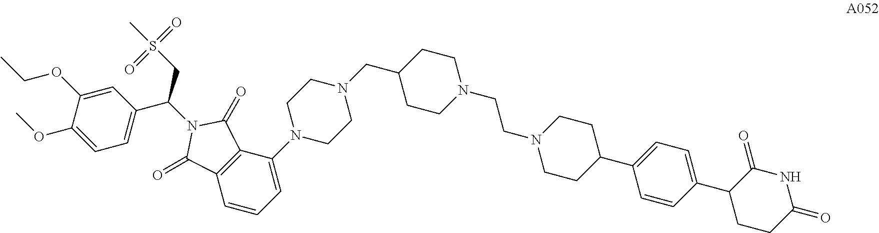 [SMILES: CCOc1cc([C@@H](CS(C)(=O)=O)N2C(=O)c3cccc(N4CCN(CC5CCN(CCN6CCC(c7ccc(C8CCC(=O)NC8=O)cc7)CC6)CC5)CC4)c3C2=O)ccc1OC]

4-((1-((1-(2-(4-(4-(2,6-Dioxopiperidin-3-yl)phenyl)piperidin-1-yl)ethyl)piperidin-4-yl)methyl)piperidin-4-yl)amino)-2-((S)-1-(3-ethoxy-4-methoxyphenyl)-2-(methylsulfonyl)-ethyl)isoindoline-1,3-dione A053.

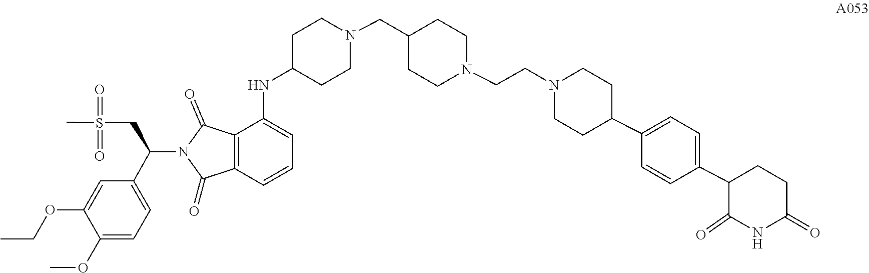 [SMILES: CCOc1cc([C@@H](CS(C)(=O)=O)N2C(=O)c3cccc(NC4CCN(CC5CCN(CCN6CCC(c7ccc(C8CCC(=O)NC8=O)cc7)CC6)CC5)CC4)c3C2=O)ccc1OC]

4-(4-(4-(4-(2,6-Dioxopiperidin-3-yl)phenyl)cyclohexyl)piperazin-1-yl)-2-((S)-1-(3-ethoxy-4-methoxyphenyl)-2-(methylsulfonyl)ethyl)isoindoline-1,3-dione A054.

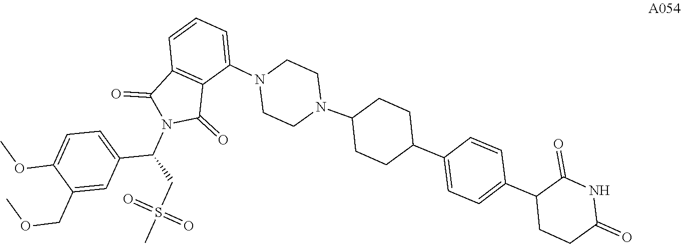 [SMILES: COCc1cc([C@@H](CS(C)(=O)=O)N2C(=O)c3cccc(N4CCN(C5CCC(c6ccc(C7CCC(=O)NC7=O)cc6)CC5)CC4)c3C2=O)ccc1OC]

5-(4-(4-(2,6-Dioxopiperidin-3-yl)phenethyl)piperazin-1-yl)-2-((S)-1-(3-ethoxy-4-methoxyphenyl)-2-(methylsulfonyl)ethyl)isoindoline-1,3-dione A055.

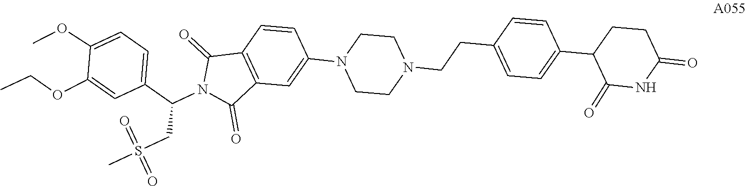 [SMILES: CCOc1cc([C@@H](CS(C)(=O)=O)N2C(=O)c3ccc(N4CCN(CCc5ccc(C6CCC(=O)NC6=O)cc5)CC4)cc3C2=O)ccc1OC]

4-(4-(1-(4-(2,6-Dioxopiperidin-3-yl)phenethyl)piperidin-4-yl)piperazin-1-yl)-2-((S)-1-(3-ethoxy-4-methoxyphenyl)-2-(methylsulfonyl)ethyl)isoindoline-1,3-dione A057.

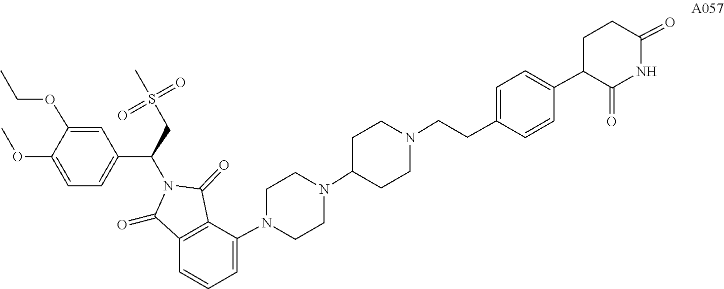 [SMILES: CCOc1cc([C@@H](CS(C)(=O)=O)N2C(=O)c3cccc(N4CCN(C5CCN(CCc6ccc(C7CCC(=O)NC7=O)cc6)CC5)CC4)c3C2=O)ccc1OC]

5-(4-(1-(4-(2,6-Dioxopiperidin-3-yl)phenethyl)piperidin-4-yl)piperazin-1-yl)-2-((S)-1-(3-ethoxy-4-methoxyphenyl)-2-(methylsulfonyl)ethyl)isoindoline-1,3-dione A058.

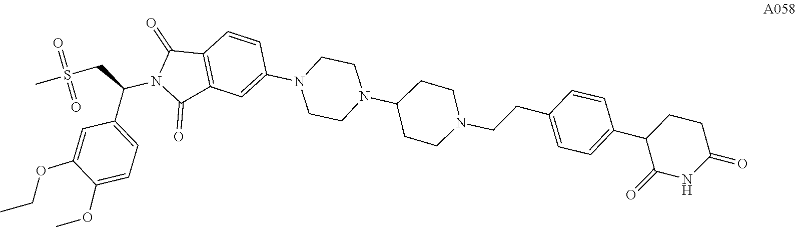 [SMILES: CCOc1cc([C@@H](CS(C)(=O)=O)N2C(=O)c3ccc(N4CCN(C5CCN(CCc6ccc(C7CCC(=O)NC7=O)cc6)CC5)CC4)cc3C2=O)ccc1OC]

5-(5-(4-(4-(2,6-Dioxopiperidin-3-yl)phenyl)-[1,4′-bipiperidin]-1′-yl)pentyl)-2-((S)-1-(3-ethoxy-4-methoxyphenyl)-2-(methylsulfonyl)ethyl)isoindoline-1,3-dione A065.

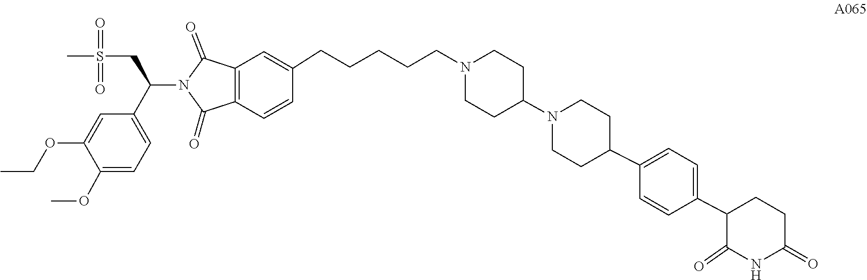 [SMILES: CCOc1cc([C@@H](CS(C)(=O)=O)N2C(=O)c3ccc(CCCCCN4CCC(N5CCC(c6ccc(C7CCC(=O)NC7=O)cc6)CC5)CC4)cc3C2=O)ccc1OC]

4-(4-(4-(4-(2,6-Dioxopiperidin-3-yl)phenyl)-[1,4′-bipiperidin]-1′-yl)butyl)-2-((S)-1-(3-ethoxy-4-methoxyphenyl)-2-(methylsulfonyl)ethyl)isoindoline-1,3-dione A067.

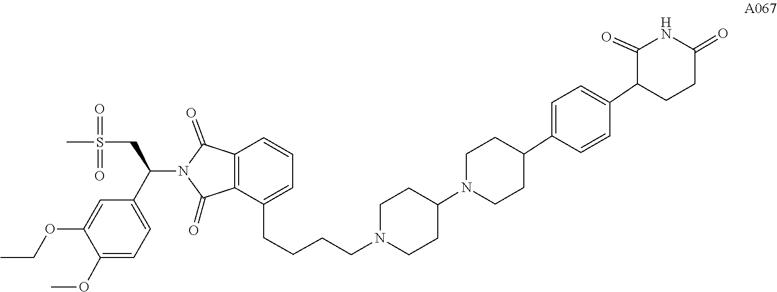 [SMILES: CCOc1cc([C@@H](CS(C)(=O)=O)N2C(=O)c3cccc(CCCCN4CCC(N5CCC(c6ccc(C7CCC(=O)NC7=O)cc6)CC5)CC4)c3C2=O)ccc1OC]

4-(3-(4-(4-(2,6-Dioxopiperidin-3-yl)phenyl)-[1,4′-bipiperidin]-1′-yl)propyl)-2-((S)-1-(3-ethoxy-4-methoxyphenyl)-2-(methylsulfonyl)ethyl)isoindoline-1,3-dione A072.

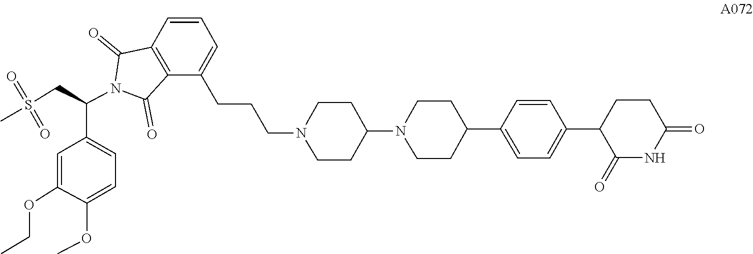 [SMILES: CCOc1cc([C@@H](CS(C)(=O)=O)N2C(=O)c3cccc(CCCN4CCC(N5CCC(c6ccc(C7CCC(=O)NC7=O)cc6)CC5)CC4)c3C2=O)ccc1OC]

4-(11-(4-(3-(2,6-Dioxopiperidin-3-yl)phenyl)piperidin-1-yl)undecyl)-2-((S)-1-(3-ethoxy-4-methoxyphenyl)-2-(methylsulfonyl)ethyl)isoindoline-1,3-dione A074.

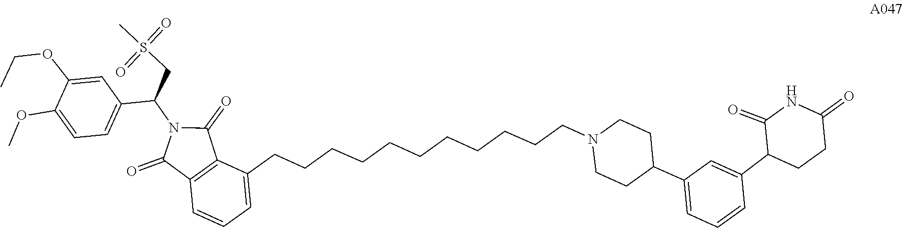 [SMILES: CCOc1cc([C@@H](CS(C)(=O)=O)N2C(=O)c3cccc(CCCCCCCCCCCN4CCC(c5cccc(C6CCC(=O)NC6=O)c5)CC4)c3C2=O)ccc1OC]

4-(11-(4-(4-(2,6-Dioxopiperidin-3-yl)phenyl)piperidin-1-yl)-1l-oxoundecyl)-2-((S)-1-(3-ethoxy-4-methoxyphenyl)-2-(methylsulfonyl)ethyl)isoindoline-1,3-dione A075.

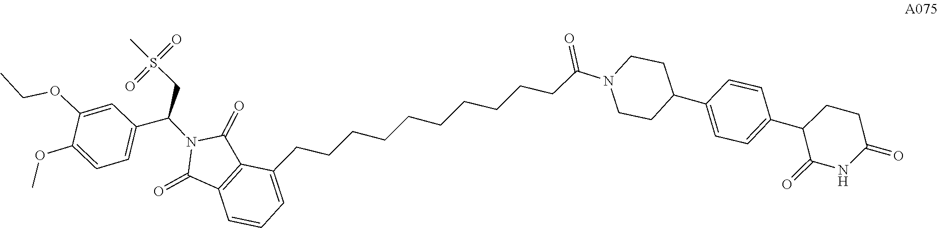 [SMILES: CCOc1cc([C@@H](CS(C)(=O)=O)N2C(=O)c3cccc(CCCCCCCCCCC(=O)N4CCC(c5ccc(C6CCC(=O)NC6=O)cc5)CC4)c3C2=O)ccc1OC]

5-(1′-(4-(4-(2,6-Dioxopiperidin-3-yl)phenoxy)benzyl)-[4,4′-bipiperidin]-1-yl)-2-((S)-1-(3-ethoxy-4-methoxyphenyl)-2-(methylsulfonyl)ethyl)isoindoline-1,3-dione A077.

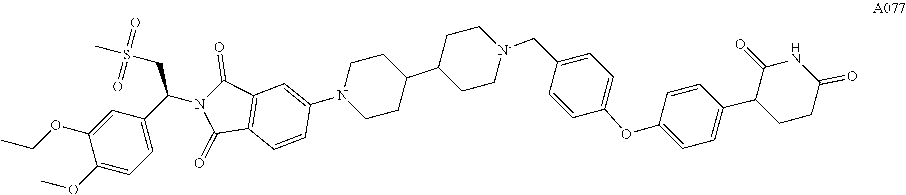

5-(5-(4-(4-((2,6-Dioxopiperidin-3-yl)amino)phenyl)-[1,4′-bipiperidin]-1′-yl)-pentyl)-2-((S)-1-(3-ethoxy-4-methoxyphenyl)-2-(methylsulfonyl)ethyl)isoindoline-1,3-dione A081.

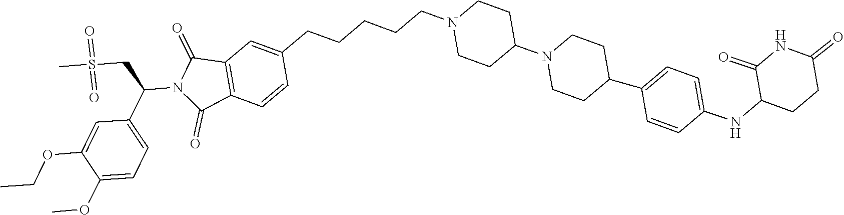 [SMILES: CCOc1cc([C@@H](CS(C)(=O)=O)N2C(=O)c3ccc(CCCCCN4CCC(N5CCC(c6ccc(NC7CCC(=O)NC7=O)cc6)CC5)CC4)cc3C2=O)ccc1OC]

5-((1-((1-(2-(4-(4-((2,6-Dioxopiperidin-3-yl)amino)-2-fluoro-5-methoxyphenyl)-piperazin-1-yl)ethyl)piperidin-4-yl)methyl)piperidin-4-yl)amino)-2-((S)-1-(3-ethoxy-4-methoxy-phenyl)-2-(methylsulfonyl)ethyl)isoindoline-1,3-dione A082.

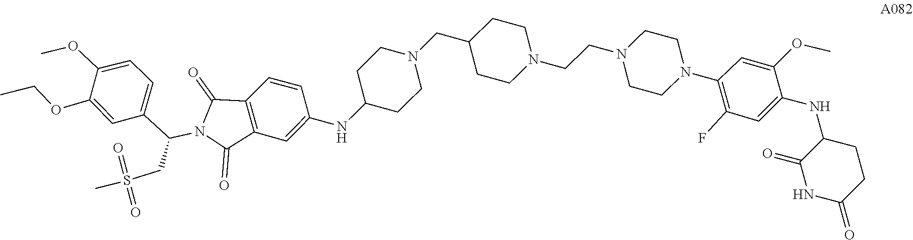 [SMILES: CCOc1cc([C@@H](CS(C)(=O)=O)N2C(=O)c3ccc(NC4CCN(CC5CCN(CCN6CCN(c7cc(OC)c(NC8CCC(=O)NC8=O)cc7F)CC6)CC5)CC4)cc3C2=O)ccc1OC]

5-(4-(2-(4-(4-((2,6-Dioxopiperidin-3-yl)amino)phenethyl)piperazin-1-yl)ethyl)-piperidin-1-yl)-2-((S)-1-(3-ethoxy-4-methoxyphenyl)-2-(methylsulfonyl)ethyl)isoindoline-1,3-dione A084.

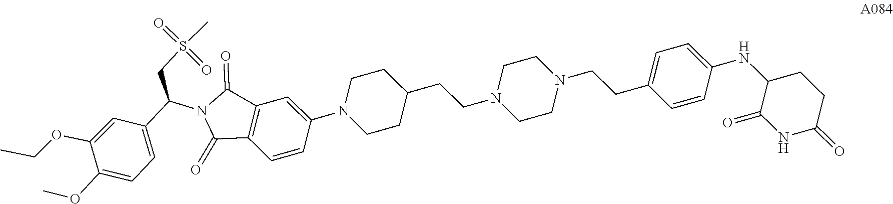 [SMILES: CCOc1cc([C@@H](CS(C)(=O)=O)N2C(=O)c3ccc(N4CCC(CCN5CCN(CCc6ccc(NC7CCC(=O)NC7=O)cc6)CC5)CC4)cc3C2=O)ccc1OC]

4-((1-((1-(2-(4-(4-((2,6-Dioxopiperidin-3-yl)amino)-2-fluorophenyl)piperidin-1-yl)ethyl)piperidin-4-yl)methyl)piperidin-4-yl)amino)-2-((S)-1-(3-ethoxy-4-methoxyphenyl)-2-(methylsulfonyl)ethyl)isoindoline-1,3-dione A085.

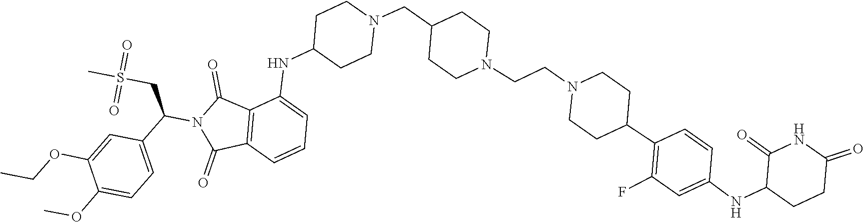 [SMILES: CCOc1cc([C@@H](CS(C)(=O)=O)N2C(=O)c3cccc(NC4CCN(CC5CCN(CCN6CCC(c7ccc(NC8CCC(=O)NC8=O)cc7F)CC6)CC5)CC4)c3C2=O)ccc1OC]

5-(1′-(4-(4-((2,6-Dioxopiperidin-3-yl)amino)phenoxy)benzyl)-[4,4′-bipiperidin]-1-yl)-2-((S)-1-(3-ethoxy-4-methoxyphenyl)-2-(methylsulfonyl)ethyl)isoindoline-1,3-dione A088.

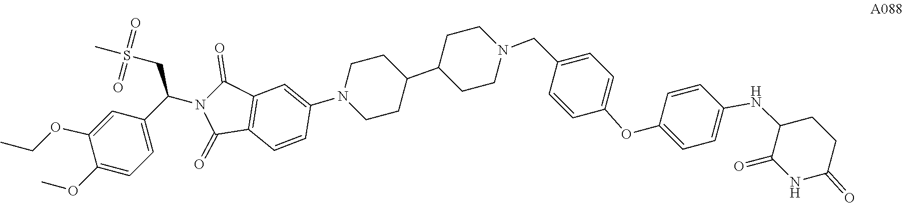 [SMILES: CCOc1cc([C@@H](CS(C)(=O)=O)N2C(=O)c3ccc(N4CCC(C5CCN(Cc6ccc(Oc7ccc(NC8CCC(=O)NC8=O)cc7)cc6)CC5)CC4)cc3C2=O)ccc1OC]

5-(1′-(4-((3-(2,6-Dioxopiperidin-3-yl)-1-methyl-1H-indazol-6-yl)oxy)benzyl)-[4,4′-bipiperidin]-1-yl)-2-((S)-1-(3-ethoxy-4-methoxyphenyl)-2-(methylsulfonyl)ethyl)-isoindoline-1,3-dione B001.

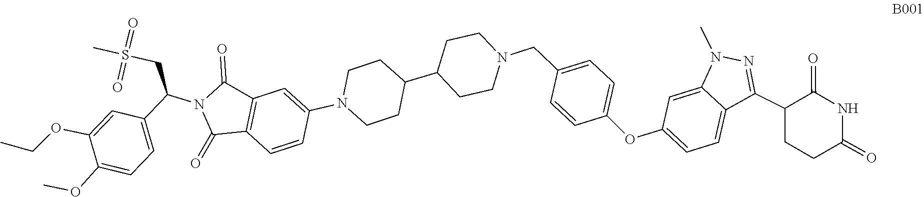 [SMILES: CCOc1cc([C@@H](CS(C)(=O)=O)N2C(=O)c3ccc(N4CCC(C5CCN(Cc6ccc(Oc7ccc8c(C9CCC(=O)NC9=O)nn(C)c8c7)cc6)CC5)CC4)cc3C2=O)ccc1OC]

5-(4-((1-(4-((3-(2,6-Dioxopiperidin-3-yl)-1-methyl-1H-indazol-6-yl)oxy)benzyl)-piperidin-4-yl)methyl)piperazin-1l-yl)-2-((S)-1-(3-ethoxy-4-methoxyphenyl)-2-(methylsulfonyl)-ethyl)isoindoline-1,3-dione B002.

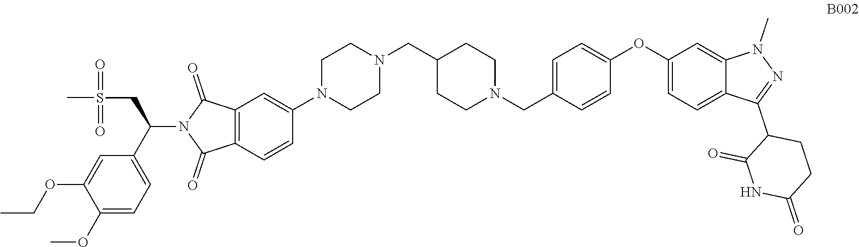 [SMILES: CCOc1cc([C@@H](CS(C)(=O)=O)N2C(=O)c3ccc(N4CCN(CC5CCN(Cc6ccc(Oc7ccc8c(C9CCC(=O)NC9=O)nn(C)c8c7)cc6)CC5)CC4)cc3C2=O)ccc1OC]

5-((4′R)-1′-(4-((3-(2,6-Dioxopiperidin-3-yl)-1-methyl-1H-indazol-6-yl)oxy)-benzyl)-3′,3′-difluoro-[4,4′-bipiperidin]-1-yl)-2-((S)-1-(3-ethoxy-4-methoxyphenyl)-2-(methyl-sulfonyl)ethyl)isoindoline-1,3-dione B008.

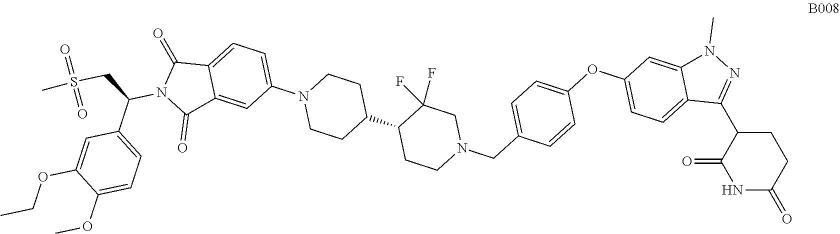 [SMILES: CCOc1cc([C@@H](CS(C)(=O)=O)N2C(=O)c3ccc(N4CCC([C@H]5CCN(Cc6ccc(Oc7ccc8c(C9CCC(=O)NC9=O)nn(C)c8c7)cc6)CC5(F)F)CC4)cc3C2=O)ccc1OC]

4-((4′R)-1′-(4-((3-(2,6-Dioxopiperidin-3-yl)-1-methyl-1H-indazol-6-yl)oxy)-benzyl)-3′,3′-difluoro-[4,4′-bipiperidin]-1-yl)-2-((S)-1-(3-ethoxy-4-methoxyphenyl)-2-(methyl-sulfonyl)ethyl)isoindoline-1,3-dione B009. ¹H NMR (400 MHz, DMSO-d₆) δ 10.90 (s, 1H), 7.73 (d, 1H), 7.61-7.67 (m, 1H), 7.26-7.34 (m, 4H), 7.24 (d, 1H), 7.08 (s, 1H), 7.01 (d, 2H), 6.93 (s, 2H), 6.85 (m, 1H), 5.75 (m, 1H), 4.37 (d, 1H), 4.35 (s, 1H), 4.06-4.14 (m, 1H), 4.01 (m, 2H), 3.92 (s, 3H), 3.72 (s, 3H), 3.64-3.72 (m, 2H), 3.49-3.59 (m, 2H), 3.00-3.07 (m, 1H), 2.99 (s, 3H), 2.86-2.92 (m, 1H), 2.81 (s, 2H), 2.61-2.73 (m, 2H), 2.31-2.39 (m, 1H), 2.21 (s, 1H), 2.13-2.20 (m, 1H), 2.03 (s, 1H), 1.81 (d, 4H), 1.74 (s, 2H), 1.48 (d, 2H), 1.31 (m, 3H); MS (ESI) m/z: 953.2 [M+H]⁺.

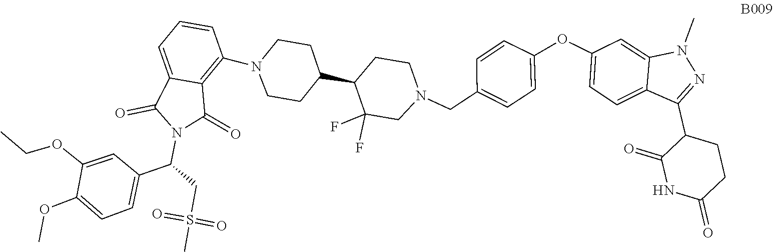 [SMILES: CCOc1cc([C@@H](CS(C)(=O)=O)N2C(=O)c3cccc(N4CCC([C@H]5CCN(Cc6ccc(Oc7ccc8c(C9CCC(=O)NC9=O)nn(C)c8c7)cc6)CC5(F)F)CC4)c3C2=O)ccc1OC]

4-(4-((4-(4-((3-(2,6-Dioxopiperidin-3-yl)-1-methyl-1H-indazol-6-yl)oxy)phenyl)-piperidin-1-yl)methyl)-4-fluoropiperidin-1-yl)-2-((S)-1-(3-ethoxy-4-methoxyphenyl)-2-(methyl-sulfonyl)ethyl)isoindoline-1,3-dione B010.

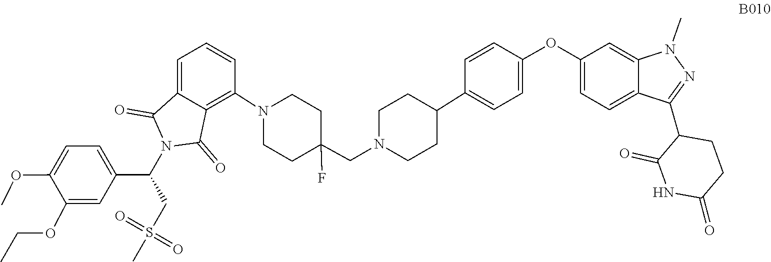 [SMILES: CCOc1cc([C@@H](CS(C)(=O)=O)N2C(=O)c3cccc(N4CCC(F)(CN5CCC(c6ccc(Oc7ccc8c(C9CCC(=O)NC9=O)nn(C)c8c7)cc6)CC5)CC4)c3C2=O)ccc1OC]

4-(1′-(2-(4-((3-(2,6-Dioxopiperidin-3-yl)-1-methyl-1H-indazol-6-yl)oxy)phenyl)-2,2-difluoroethyl)-[4,4′-bipiperidin]-1-yl)-2-((S)-1-(3-ethoxy-4-methoxyphenyl)-2-(methyl-sulfonyl)ethyl)isoindoline-1,3-dione B011.

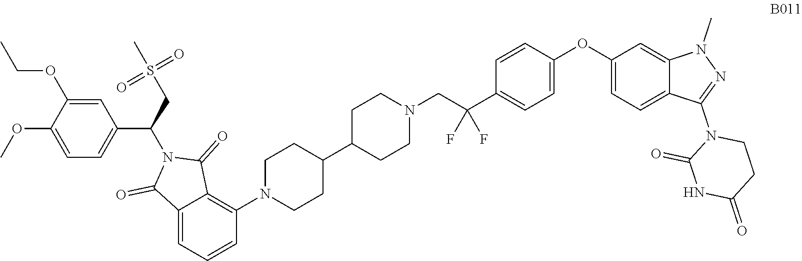 [SMILES: CCOc1cc([C@@H](CS(C)(=O)=O)N2C(=O)c3cccc(N4CCC(C5CCN(CC(F)(F)c6ccc(Oc7ccc8c(N9CCC(=O)NC9=O)nn(C)c8c7)cc6)CC5)CC4)c3C2=O)ccc1OC]

4-(4-(2-(4-(4-((3-(2,6-Dioxopiperidin-3-yl)-1-methyl-1H-indazol-6-yl)oxy)-phenyl)piperazin-1-yl)ethyl)piperidin-1-yl)-2-((S)-1-(3-ethoxy-4-methoxyphenyl)-2-(methyl-sulfonyl)ethyl)isoindoline-1,3-dione B012.

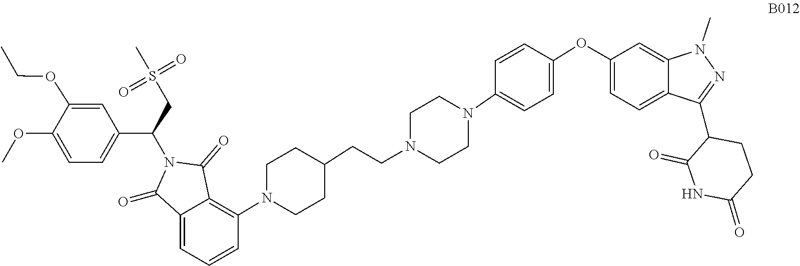 [SMILES: CCOc1cc([C@@H](CS(C)(=O)=O)N2C(=O)c3cccc(N4CCC(CCN5CCN(c6ccc(Oc7ccc8c(C9CCC(=O)NC9=O)nn(C)c8c7)cc6)CC5)CC4)c3C2=O)ccc1OC]

5-((R)-1′-(4-((3-(2,4-Dioxotetrahydropyrimidin-1(2H)-yl)-1-methyl-1H-indazol-6-yl)oxy)benzyl)-3′,3′-difluoro-[4,4′-bipiperidin]-1-yl)-2-((S)-1-(3-ethoxy-4-methoxyphenyl)-2-(methylsulfonyl)ethyl)isoindoline-1,3-dione B151. ¹H NMR (400 MHz, DMSO-d₆) δ 10.48-10.67 (m, 1H), 7.66 (d, 1H), 7.60 (d, 1H), 7.30 (d, 2H), 7.21-7.27 (m, 2H), 7.19-7.21 (m, 1H), 7.07 (s, 1H), 7.00 (d, 2H), 6.92 (s, 2H), 6.84-6.89 (m, 1H), 5.72-5.77 (m, 1H), 4.34-4.38 (m, 1H), 3.98-4.11 (m, 5H), 3.96-3.84 (m, 5H), 3.72 (s, 3H), 3.47-3.58 (m, 2H), 2.99 (s, 3H), 2.82-2.95 (m, 4H), 2.71-2.80 (m, 3H), 2.13-2.27 (m, 1H), 1.89-2.04 (m, 2H), 1.75-1.83 (m, 2H), 1.63-1.73 (m, 1H), 1.37-1.48 (m, 2H), 1.31 (t, 3H), 1.02-1.29 (m, 1H); MS (ESI) m/z: 954.5 [M+H]⁺.

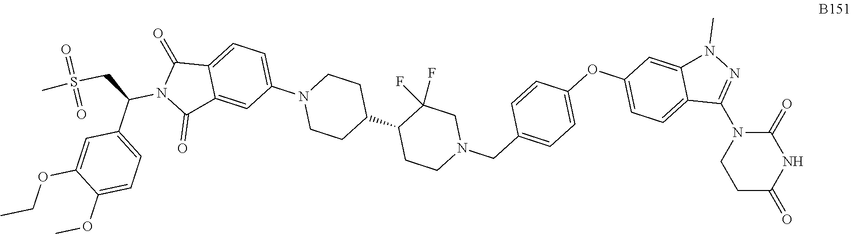 [SMILES: CCOc1cc([C@@H](CS(C)(=O)=O)N2C(=O)c3ccc(N4CCC([C@H]5CCN(Cc6ccc(Oc7ccc8c(N9CCC(=O)NC9=O)nn(C)c8c7)cc6)CC5(F)F)CC4)cc3C2=O)ccc1OC]

4-((R)-1′-(4-((3-(2,4-Dioxotetrahydropyrimidin-1(2H)-yl)-5-fluoro-1-methyl-1H-indazol-6-yl)oxy)benzyl)-3′,3′-difluoro-[4,4′-bipiperidin]-1-yl)-2-((S)-1-(3-ethoxy-4-methoxy-phenyl)-2-(methylsulfonyl)ethyl)isoindoline-1,3-dione B152.

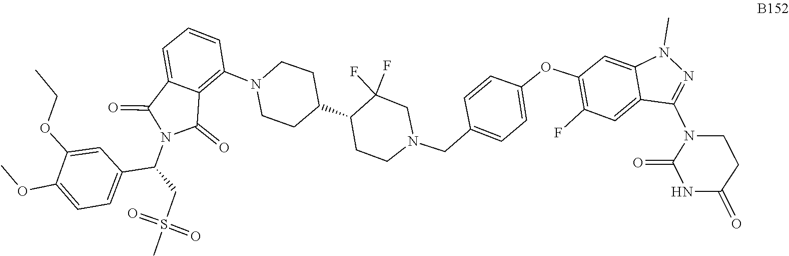 [SMILES: CCOc1cc([C@@H](CS(C)(=O)=O)N2C(=O)c3cccc(N4CCC([C@H]5CCN(Cc6ccc(Oc7cc8c(cc7F)c(N7CCC(=O)NC7=O)nn8C)cc6)CC5(F)F)CC4)c3C2=O)ccc1OC]

4-(4-((R)-1-(4-((3-(2,4-Dioxotetrahydropyrimidin-1(2H)-yl)-5-fluoro-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)isoindoline-1,3-dione B153.

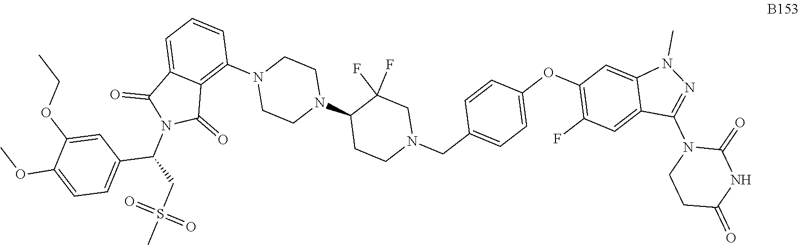 [SMILES: CCOc1cc([C@@H](CS(C)(=O)=O)N2C(=O)c3cccc(N4CCN([C@@H]5CCN(Cc6ccc(Oc7cc8c(cc7F)c(N7CCC(=O)NC7=O)nn8C)cc6)CC5(F)F)CC4)c3C2=O)ccc1OC]

The following compounds are prepared similarly according to the synthetic procedures or methodologies exemplified herein.

5-(2-(2-(4-(4-(2,6-Dioxopiperidin-3-yl)phenyl)-[1,4′-bipiperidin]-1′-yl)ethoxy)-ethyl)-2-((S)-1-(3-ethoxy-4-methoxyphenyl)-2-(methylsulfonyl)ethyl)isoindoline-1,3-dione A006.

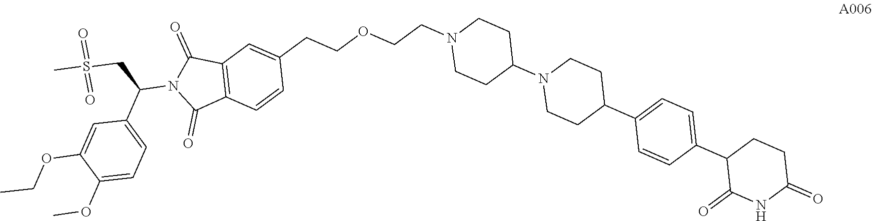 [SMILES: CCOc1cc([C@@H](CS(C)(=O)=O)N2C(=O)c3ccc(CCOCCN4CCC(N5CCC(c6ccc(C7CCC(=O)NC7=O)cc6)CC5)CC4)cc3C2=O)ccc1OC]

5-(4-(1′-(3-(4-(2,6-Dioxopiperidin-3-yl)phenyl)propyl)-[1,4′-bipiperidin]-4-yl)-piperazin-1-yl)-2-((S)-1-(3-ethoxy-4-methoxyphenyl)-2-(methylsulfonyl)ethyl)isoindoline-1,3-dione A012.

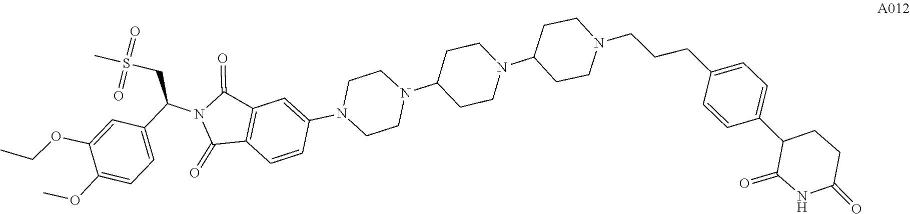 [SMILES: CCOc1cc([C@@H](CS(C)(=O)=O)N2C(=O)c3ccc(N4CCN(C5CCN(C6CCN(CCCc7ccc(C8CCC(=O)NC8=O)cc7)CC6)CC5)CC4)cc3C2=O)ccc1OC]

4-(4-(1′-(3-(4-(2,6-Dioxopiperidin-3-yl)phenyl)propyl)-[1,4′-bipiperidin]-4-yl)-piperazin-1-yl)-2-((S)-1-(3-ethoxy-4-methoxyphenyl)-2-(methylsulfonyl)ethyl)isoindoline-1,3-dione A013.

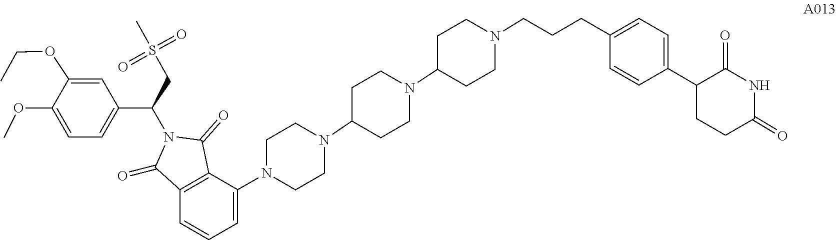 [SMILES: CCOc1cc([C@@H](CS(C)(=O)=O)N2C(=O)c3cccc(N4CCN(C5CCN(C6CCN(CCCc7ccc(C8CCC(=O)NC8=O)cc7)CC6)CC5)CC4)c3C2=O)ccc1OC]

4-(4-(1′-(4-(2,6-Dioxopiperidin-3-yl)phenethyl)-[1,4′-bipiperidin]-4-yl)piperazin-1-yl)-2-((S)-1-(3-ethoxy-4-methoxyphenyl)-2-(methylsulfonyl)ethyl)isoindoline-1,3-dione A014.

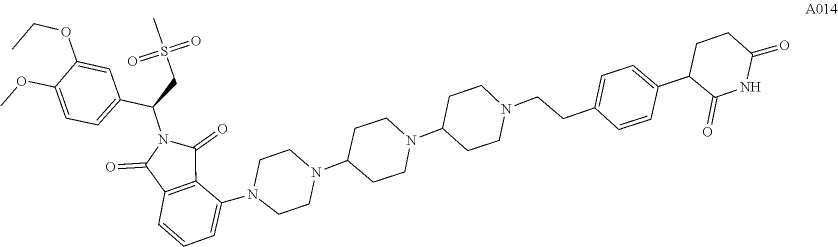 [SMILES: CCOc1cc([C@@H](CS(C)(=O)=O)N2C(=O)c3cccc(N4CCN(C5CCN(C6CCN(CCc7ccc(C8CCC(=O)NC8=O)cc7)CC6)CC5)CC4)c3C2=O)ccc1OC]

5-(4-(1′-(4-(2,6-Dioxopiperidin-3-yl)phenethyl)-[1,4′-bipiperidin]-4-yl)piperazin-1-yl)-2-((S)-1-(3-ethoxy-4-methoxyphenyl)-2-(methylsulfonyl)ethyl)isoindoline-1,3-dione A015.

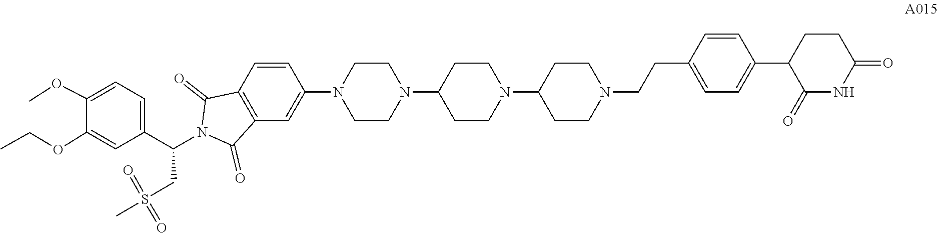 [SMILES: CCOc1cc([C@@H](CS(C)(=O)=O)N2C(=O)c3ccc(N4CCN(C5CCN(C6CCN(CCc7ccc(C8CCC(=O)NC8=O)cc7)CC6)CC5)CC4)cc3C2=O)ccc1OC]

4-(4-(4-(2,6-Dioxopiperidin-3-yl)phenyl)-[1,4′-bipiperidin]-1′-yl)-N-(2-((S)-1-(3-ethoxy-4-methoxyphenyl)-2-(methylsulfonyl)ethyl)-1,3-dioxoisoindolin-4-yl)butanamide A026.

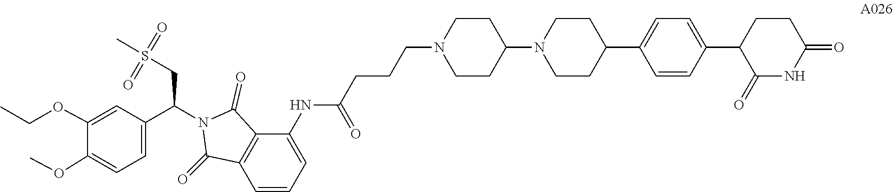 [SMILES: CCOc1cc([C@@H](CS(C)(=O)=O)N2C(=O)c3cccc(NC(=O)CCCN4CCC(N5CCC(c6ccc(C7CCC(=O)NC7=O)cc6)CC5)CC4)c3C2=O)ccc1OC]

4-(4-(2-(4-(2,6-Dioxopiperidin-3-yl)phenyl)-2-azaspiro[3.3]heptan-6-yl)-piperazin-1-yl)-2-((S)-1-(3-ethoxy-4-methoxyphenyl)-2-(methylsulfonyl)ethyl)isoindoline-1,3-dione A048.

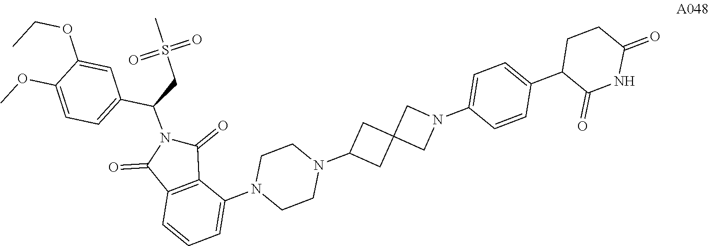 [SMILES: CCOc1cc([C@@H](CS(C)(=O)=O)N2C(=O)c3cccc(N4CCN(C5CC6(C5)CN(c5ccc(C7CCC(=O)NC7=O)cc5)C6)CC4)c3C2=O)ccc1OC]

5-(4-(2-(4-(2,6-Dioxopiperidin-3-yl)phenyl)-2-azaspiro[3.3]heptan-6-yl)-piperazin-1-yl)-2-((S)-1-(3-ethoxy-4-methoxyphenyl)-2-(methylsulfonyl)ethyl)isoindoline-1,3-dione A049.

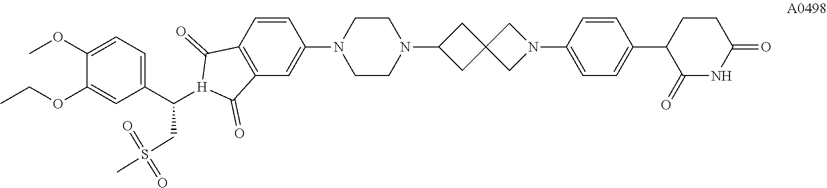

5-(1-(4-(4-(4-(2,6-Dioxopiperidin-3-yl)phenyl)-[1,4′-bipiperidin]-1′-yl)butyl)-piperidin-4-yl)-2-((1S)-1-(3-ethoxy-4-methoxyphenyl)-2-(methylsulfinyl)ethyl)isoindoline-1,3-dione A059.

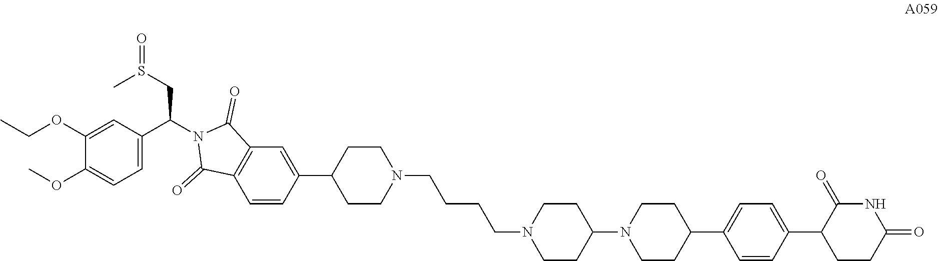 [SMILES: CCOc1cc([C@@H](CS(C)=O)N2C(=O)c3ccc(C4CCN(CCCCN5CCC(N6CCC(c7ccc(C8CCC(=O)NC8=O)cc7)CC6)CC5)CC4)cc3C2=O)ccc1OC]

5-(1-(2-(4-(4-(2,6-Dioxopiperidin-3-yl)phenyl)-[1,4′-bipiperidin]-1′-yl)ethyl)-piperidin-4-yl)-2-((S)-1-(3-ethoxy-4-methoxyphenyl)-2-(methylsulfonyl)ethyl)isoindoline-1,3-dione A060.

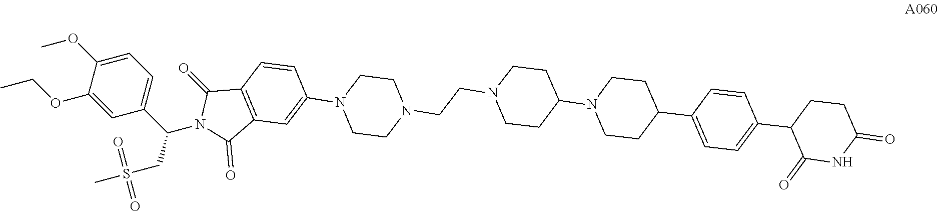 [SMILES: CCOc1cc([C@@H](CS(C)(=O)=O)N2C(=O)c3ccc(N4CCN(CCN5CCC(N6CCC(c7ccc(C8CCC(=O)NC8=O)cc7)CC6)CC5)CC4)cc3C2=O)ccc1OC]

5-(1-(3-(4-(4-(2,6-Dioxopiperidin-3-yl)phenyl)-[1,4′-bipiperidin]-1′-yl)propyl)-piperidin-4-yl)-2-((S)-1-(3-ethoxy-4-methoxyphenyl)-2-(methylsulfonyl)ethyl)isoindoline-1,3-dione A061.

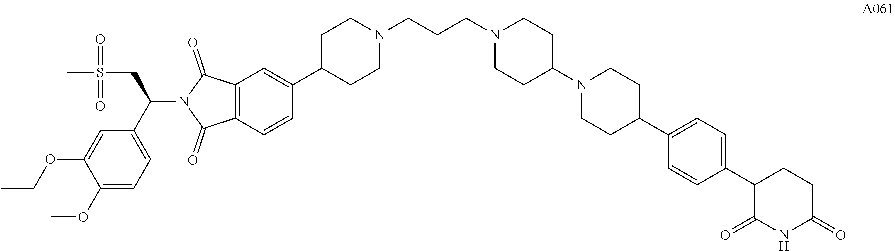 [SMILES: CCOc1cc([C@@H](CS(C)(=O)=O)N2C(=O)c3ccc(C4CCN(CCCN5CCC(N6CCC(c7ccc(C8CCC(=O)NC8=O)cc7)CC6)CC5)CC4)cc3C2=O)ccc1OC]

4-(1-(2-(4-(4-(2,6-Dioxopiperidin-3-yl)phenyl)-[1,4′-bipiperidin]-1′-yl)ethyl)-piperidin-4-yl)-2-((S)-1-(3-ethoxy-4-methoxyphenyl)-2-(methylsulfonyl)ethyl)isoindoline-1,3-dione A062.

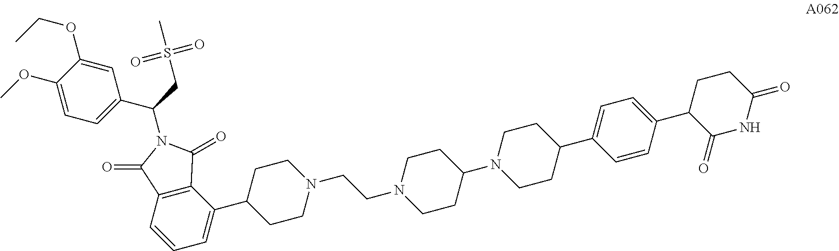 [SMILES: CCOc1cc([C@@H](CS(C)(=O)=O)N2C(=O)c3cccc(C4CCN(CCN5CCC(N6CCC(c7ccc(C8CCC(=O)NC8=O)cc7)CC6)CC5)CC4)c3C2=O)ccc1OC]

4-(1-(3-(4-(4-(2,6-Dioxopiperidin-3-yl)phenyl)-[1,4′-bipiperidin]-1′-yl)propyl)-piperidin-4-yl)-2-((S)-1-(3-ethoxy-4-methoxyphenyl)-2-(methylsulfonyl)ethyl)isoindoline-1,3-dione A063.

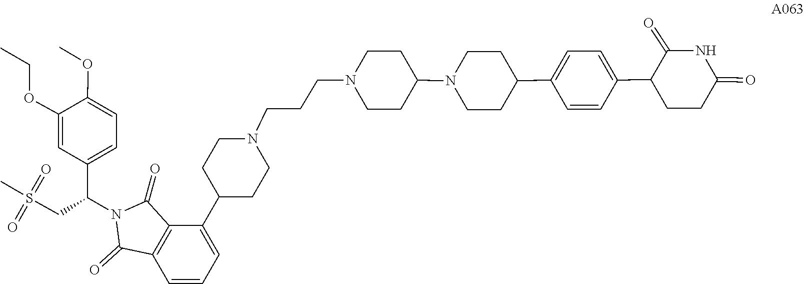 [SMILES: CCOc1cc([C@@H](CS(C)(=O)=O)N2C(=O)c3cccc(C4CCN(CCCN5CCC(N6CCC(c7ccc(C8CCC(=O)NC8=O)cc7)CC6)CC5)CC4)c3C2=O)ccc1OC]

4-(1-(4-(4-(4-(2,6-Dioxopiperidin-3-yl)phenyl)-[1,4′-bipiperidin]-1′-yl)butyl)-piperidin-4-yl)-2-((S)-1-(3-ethoxy-4-methoxyphenyl)-2-(methylsulfonyl)ethyl)isoindoline-1,3-dione A064.

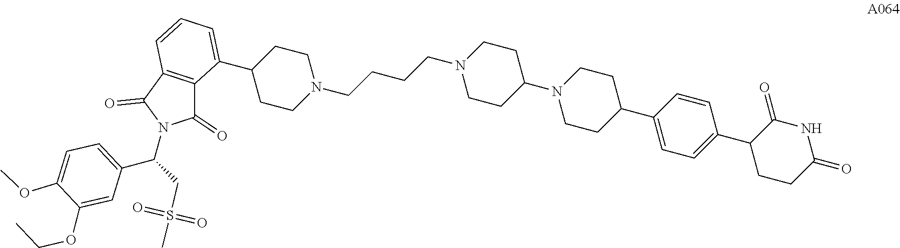 [SMILES: CCOc1cc([C@@H](CS(C)(=O)=O)N2C(=O)c3cccc(C4CCN(CCCCN5CCC(N6CCC(c7ccc(C8CCC(=O)NC8=O)cc7)CC6)CC5)CC4)c3C2=O)ccc1OC]

5-(4-(4-(4-(2,6-Dioxopiperidin-3-yl)phenyl)-[1,4′-bipiperidin]-1′-yl)butyl)-2-((S)-1-(3-ethoxy-4-methoxyphenyl)-2-(methylsulfonyl)ethyl)isoindoline-1,3-dione A066.

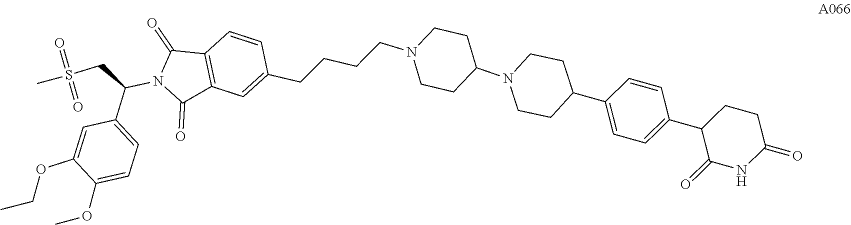 [SMILES: CCOc1cc([C@@H](CS(C)(=O)=O)N2C(=O)c3ccc(CCCCN4CCC(N5CCC(c6ccc(C7CCC(=O)NC7=O)cc6)CC5)CC4)cc3C2=O)ccc1OC]

5-(2-(4-(4-(2,6-Dioxopiperidin-3-yl)phenyl)-[1,4′-bipiperidin]-1′-yl)ethyl)-2-((S)-1-(3-ethoxy-4-methoxyphenyl)-2-(methylsulfonyl)ethyl)isoindoline-1,3-dione A069.

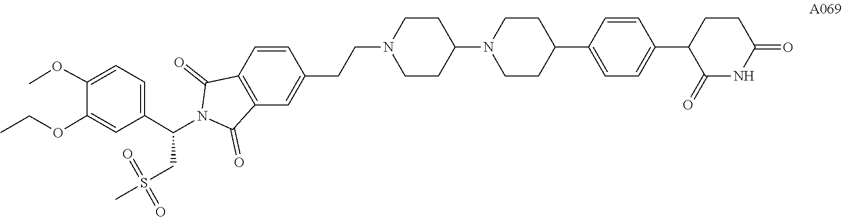 [SMILES: CCOc1cc([C@@H](CS(C)(=O)=O)N2C(=O)c3ccc(CCN4CCC(N5CCC(c6ccc(C7CCC(=O)NC7=O)cc6)CC5)CC4)cc3C2=O)ccc1OC]

5-(3-(4-(4-(2,6-Dioxopiperidin-3-yl)phenyl)-[1,4′-bipiperidin]-1′-yl)propyl)-2-((S)-1-(3-ethoxy-4-methoxyphenyl)-2-(methylsulfonyl)ethyl)isoindoline-1,3-dione A070.

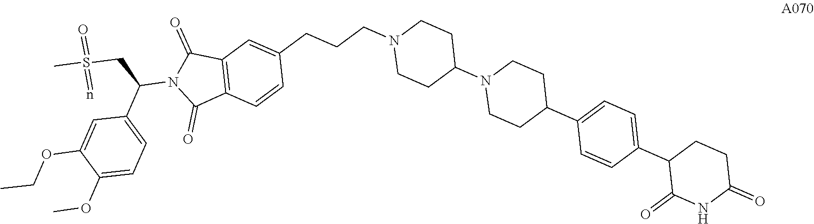

4-(2-(4-(4-(2,6-Dioxopiperidin-3-yl)phenyl)-[1,4′-bipiperidin]-1′-yl)ethyl)-2-((S)-1-(3-ethoxy-4-methoxyphenyl)-2-(methylsulfonyl)ethyl)isoindoline-1,3-dione A071.

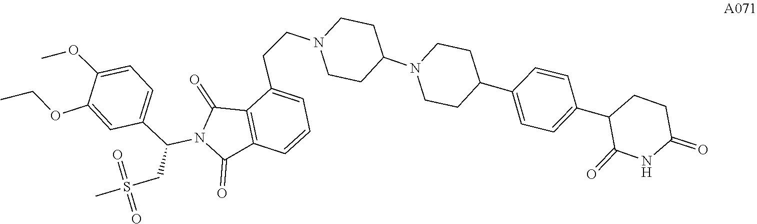 [SMILES: CCOc1cc([C@@H](CS(C)(=O)=O)N2C(=O)c3cccc(CCN4CCC(N5CCC(c6ccc(C7CCC(=O)NC7=O)cc6)CC5)CC4)c3C2=O)ccc1OC]

4-(6-(4-(2-(4-(4-(2,6-Dioxopiperidin-3-yl)phenyl)piperidin-1-yl)-2-oxoethyl)-piperidin-1-yl)hexyl)-2-((S)-1-(3-ethoxy-4-methoxyphenyl)-2-(methylsulfonyl)ethyl)isoindoline-1,3-dione A073.

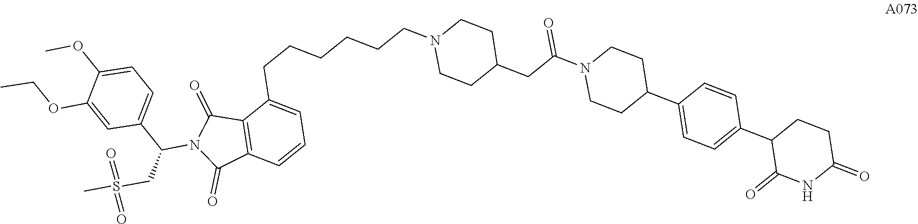 [SMILES: CCOc1cc([C@@H](CS(C)(=O)=O)N2C(=O)c3cccc(CCCCCCN4CCC(CC(=O)N5CCC(c6ccc(C7CCC(=O)NC7=O)cc6)CC5)CC4)c3C2=O)ccc1OC]

5-(4-(1-((1-(4-((2,6-Dioxopiperidin-3-yl)amino)phenyl)piperidin-4-yl)methyl)-piperidin-4-yl)piperazin-1-yl)-2-((S)-1-(3-ethoxy-4-methoxyphenyl)-2-(methylsulfonyl)ethyl)-isoindoline-1,3-dione A083.

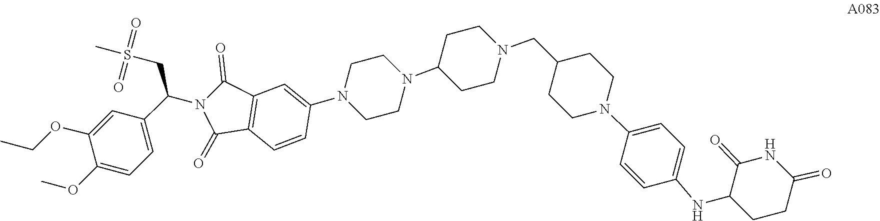 [SMILES: CCOc1cc([C@@H](CS(C)(=O)=O)N2C(=O)c3ccc(N4CCN(C5CCN(CC6CCN(c7ccc(NC8CCC(=O)NC8=O)cc7)CC6)CC5)CC4)cc3C2=O)ccc1OC]

5-(4-((1-(2-((4-((2,6-Dioxopiperidin-3-yl)amino)phenyl)amino)ethyl)piperidin-4-yl)methyl)piperazin-1-yl)-2-((S)-1-(3-ethoxy-4-methoxyphenyl)-2-(methylsulfonyl)ethyl)-isoindoline-1,3-dione A087.

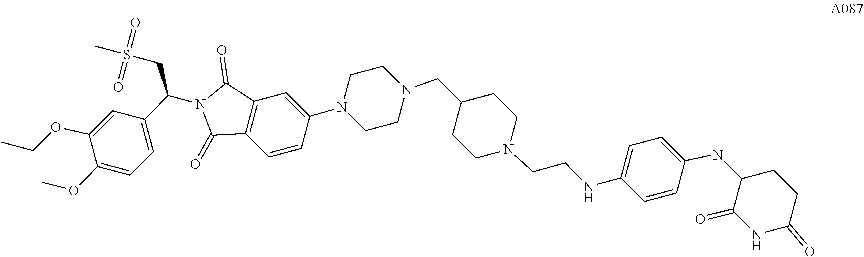

5-(1′-(4-((3-((S)-2,6-Dioxopiperidin-3-yl)-1-methyl-1H-indazol-6-yl)oxy)benzyl)-[4,4′-bipiperidin]-1-yl)-2-((S)-1-(3-ethoxy-4-methoxyphenyl)-2-(methylsulfonyl)ethyl)-isoindoline-1,3-dione B021.

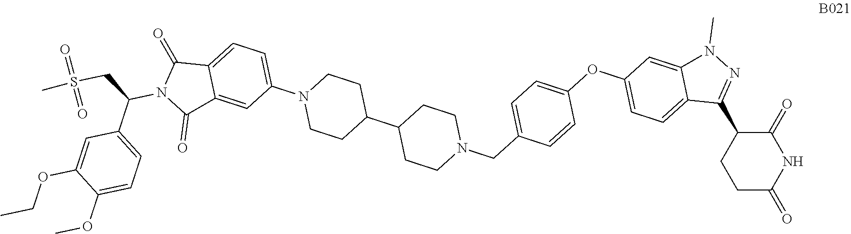 [SMILES: CCOc1cc([C@@H](CS(C)(=O)=O)N2C(=O)c3ccc(N4CCC(C5CCN(Cc6ccc(Oc7ccc8c([C@@H]9CCC(=O)NC9=O)nn(C)c8c7)cc6)CC5)CC4)cc3C2=O)ccc1OC]

5-(1′-(4-((3-((R)-2,6-Dioxopiperidin-3-yl)-1-methyl-1H-indazol-6-yl)oxy)benzyl)-[4,4′-bipiperidin]-1-yl)-2-((S)-1-(3-ethoxy-4-methoxyphenyl)-2-(methylsulfonyl)ethyl)-isoindoline-1,3-dione B022.

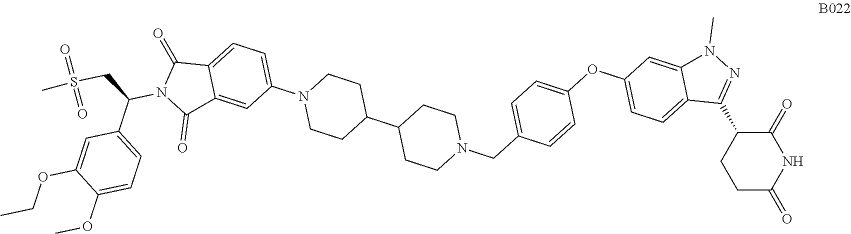 [SMILES: CCOc1cc([C@@H](CS(C)(=O)=O)N2C(=O)c3ccc(N4CCC(C5CCN(Cc6ccc(Oc7ccc8c([C@H]9CCC(=O)NC9=O)nn(C)c8c7)cc6)CC5)CC4)cc3C2=O)ccc1OC]

5-(4-((S)-3,3-Difluoro-1-(4-((1-methyl-3-((S)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)isoindoline-1,3-dione B023.

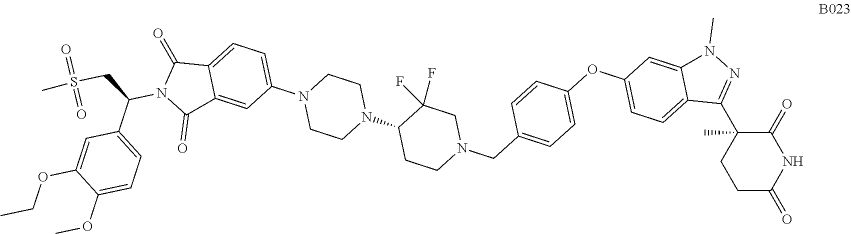 [SMILES: CCOc1cc([C@@H](CS(C)(=O)=O)N2C(=O)c3ccc(N4CCN([C@H]5CCN(Cc6ccc(Oc7ccc8c([C@]9(C)CCC(=O)NC9=O)nn(C)c8c7)cc6)CC5(F)F)CC4)cc3C2=O)ccc1OC]

5-(4-((S)-3,3-Difluoro-1-(4-((1-methyl-3-((R)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)isoindoline-1,3-dione B024.

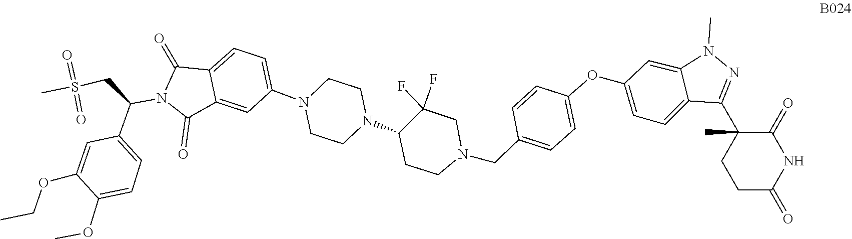 [SMILES: CCOc1cc([C@@H](CS(C)(=O)=O)N2C(=O)c3ccc(N4CCN([C@H]5CCN(Cc6ccc(Oc7ccc8c([C@@]9(C)CCC(=O)NC9=O)nn(C)c8c7)cc6)CC5(F)F)CC4)cc3C2=O)ccc1OC]

5-(4-((S)-3,3-Difluoro-1-(4-((5-fluoro-1-methyl-3-((S)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)isoindoline-1,3-dione B025.

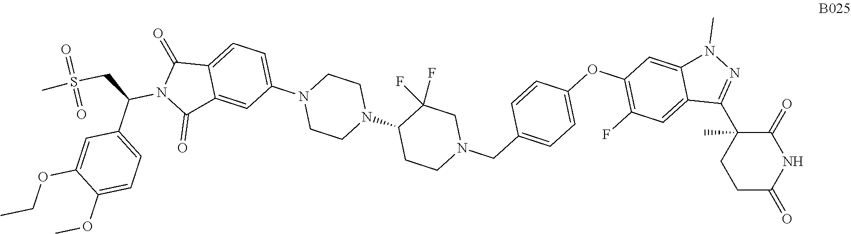 [SMILES: CCOc1cc([C@@H](CS(C)(=O)=O)N2C(=O)c3ccc(N4CCN([C@H]5CCN(Cc6ccc(Oc7cc8c(cc7F)c([C@]7(C)CCC(=O)NC7=O)nn8C)cc6)CC5(F)F)CC4)cc3C2=O)ccc1OC]

5-(4-((S)-3,3-Difluoro-1-(4-((5-fluoro-1-methyl-3-((R)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)isoindoline-1,3-dione B026.

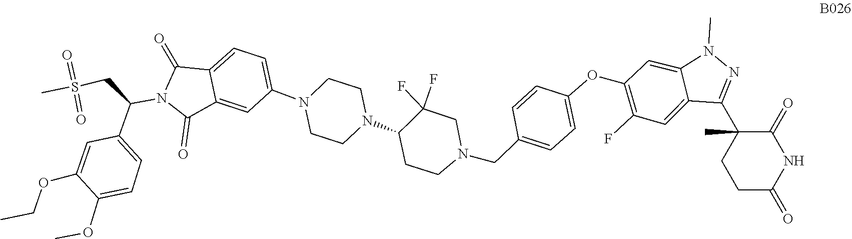 [SMILES: CCOc1cc([C@@H](CS(C)(=O)=O)N2C(=O)c3ccc(N4CCN([C@H]5CCN(Cc6ccc(Oc7cc8c(cc7F)c([C@@]7(C)CCC(=O)NC7=O)nn8C)cc6)CC5(F)F)CC4)cc3C2=O)ccc1OC]

5-(4-((R)-3,3-Difluoro-1-(4-((1-methyl-3-((S)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)isoindoline-1,3-dione B027.

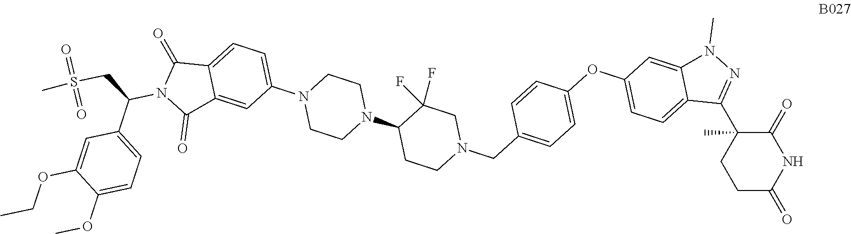 [SMILES: CCOc1cc([C@@H](CS(C)(=O)=O)N2C(=O)c3ccc(N4CCN([C@@H]5CCN(Cc6ccc(Oc7ccc8c([C@]9(C)CCC(=O)NC9=O)nn(C)c8c7)cc6)CC5(F)F)CC4)cc3C2=O)ccc1OC]

5-(4-((R)-3,3-Difluoro-1-(4-((1-methyl-3-((R)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)isoindoline-1,3-dione B028.

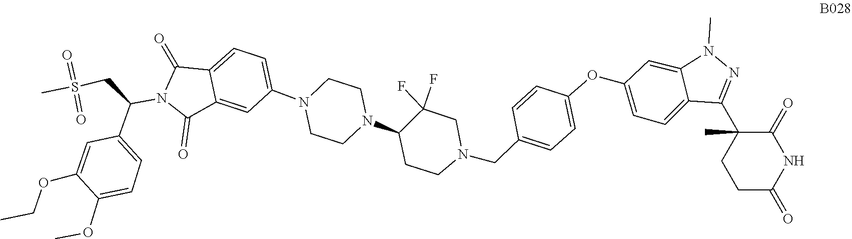 [SMILES: CCOc1cc([C@@H](CS(C)(=O)=O)N2C(=O)c3ccc(N4CCN([C@@H]5CCN(Cc6ccc(Oc7ccc8c([C@@]9(C)CCC(=O)NC9=O)nn(C)c8c7)cc6)CC5(F)F)CC4)cc3C2=O)ccc1OC]

5-(4-((R)-3,3-Difluoro-1-(4-((5-fluoro-1-methyl-3-((S)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)isoindoline-1,3-dione B029.

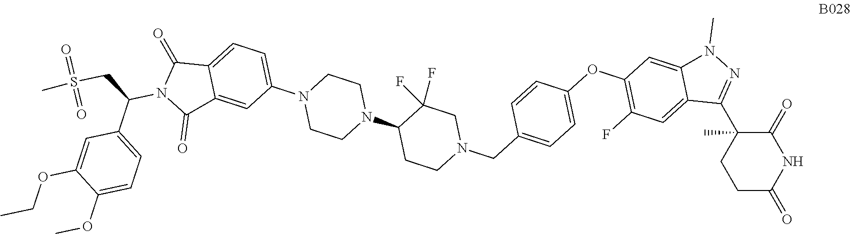 [SMILES: CCOc1cc([C@@H](CS(C)(=O)=O)N2C(=O)c3ccc(N4CCN([C@@H]5CCN(Cc6ccc(Oc7cc8c(cc7F)c([C@]7(C)CCC(=O)NC7=O)nn8C)cc6)CC5(F)F)CC4)cc3C2=O)ccc1OC]

5-(4-((R)-3,3-Difluoro-1-(4-((5-fluoro-1-methyl-3-((R)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)isoindoline-1,3-dione B030.

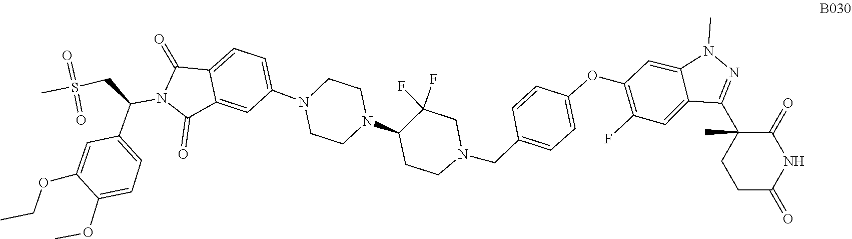 [SMILES: CCOc1cc([C@@H](CS(C)(=O)=O)N2C(=O)c3ccc(N4CCN([C@@H]5CCN(Cc6ccc(Oc7cc8c(cc7F)c([C@@]7(C)CCC(=O)NC7=O)nn8C)cc6)CC5(F)F)CC4)cc3C2=O)ccc1OC]

4-(4-((S)-3,3-Difluoro-1-(4-((1-methyl-3-((S)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)isoindoline-1,3-dione B031.

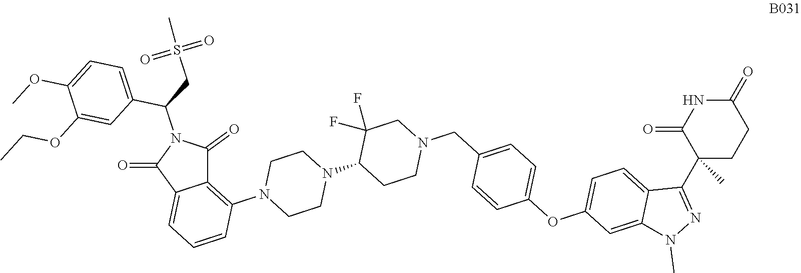 [SMILES: CCOc1cc([C@@H](CS(C)(=O)=O)N2C(=O)c3cccc(N4CCN([C@H]5CCN(Cc6ccc(Oc7ccc8c([C@]9(C)CCC(=O)NC9=O)nn(C)c8c7)cc6)CC5(F)F)CC4)c3C2=O)ccc1OC]

4-(4-((S)-3,3-Difluoro-1-(4-((1-methyl-3-((R)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)isoindoline-1,3-dione B032.

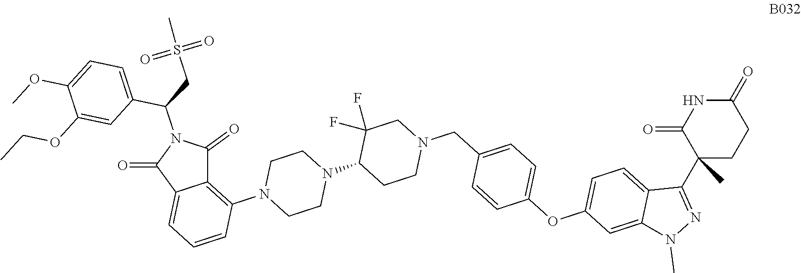 [SMILES: CCOc1cc([C@@H](CS(C)(=O)=O)N2C(=O)c3cccc(N4CCN([C@H]5CCN(Cc6ccc(Oc7ccc8c([C@@]9(C)CCC(=O)NC9=O)nn(C)c8c7)cc6)CC5(F)F)CC4)c3C2=O)ccc1OC]

4-(4-((S)-3,3-Difluoro-1-(4-((5-fluoro-1-methyl-3-((S)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)isoindoline-1,3-dione B033.

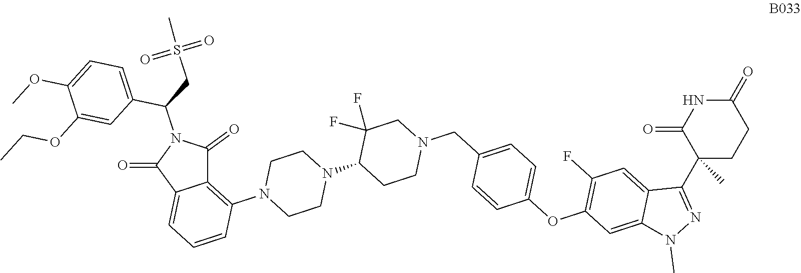 [SMILES: CCOc1cc([C@@H](CS(C)(=O)=O)N2C(=O)c3cccc(N4CCN([C@H]5CCN(Cc6ccc(Oc7cc8c(cc7F)c([C@]7(C)CCC(=O)NC7=O)nn8C)cc6)CC5(F)F)CC4)c3C2=O)ccc1OC]

4-(4-((S)-3,3-Difluoro-1-(4-((5-fluoro-1-methyl-3-((S)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)isoindoline-1,3-dione B034.

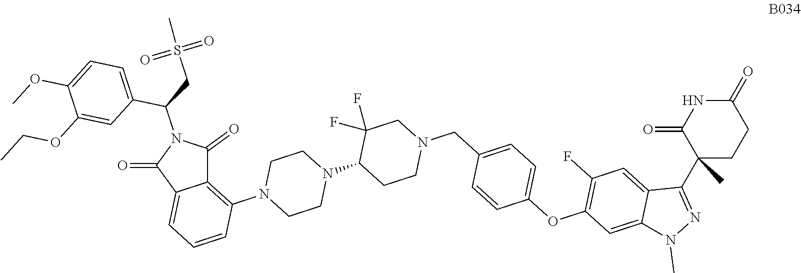 [SMILES: CCOc1cc([C@@H](CS(C)(=O)=O)N2C(=O)c3cccc(N4CCN([C@H]5CCN(Cc6ccc(Oc7cc8c(cc7F)c([C@@]7(C)CCC(=O)NC7=O)nn8C)cc6)CC5(F)F)CC4)c3C2=O)ccc1OC]

4-(4-((R)-3,3-Difluoro-1-(4-((1-methyl-3-((S)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)isoindoline-1,3-dione B035.

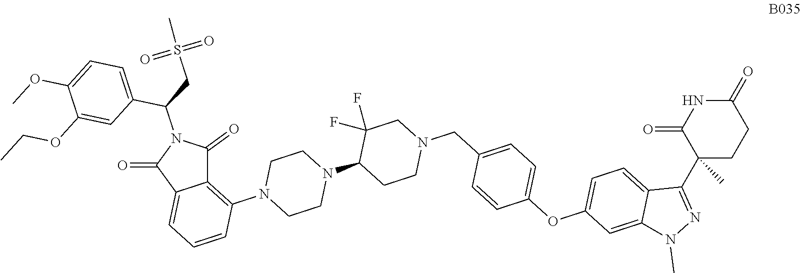 [SMILES: CCOc1cc([C@@H](CS(C)(=O)=O)N2C(=O)c3cccc(N4CCN([C@@H]5CCN(Cc6ccc(Oc7ccc8c([C@]9(C)CCC(=O)NC9=O)nn(C)c8c7)cc6)CC5(F)F)CC4)c3C2=O)ccc1OC]

4-(4-((R)-3,3-Difluoro-1-(4-((1-methyl-3-((R)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)isoindoline-1,3-dione B036.

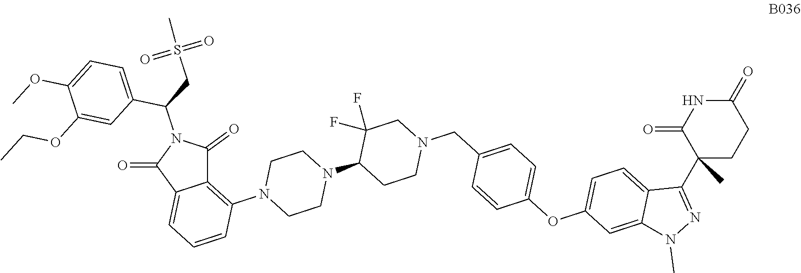 [SMILES: CCOc1cc([C@@H](CS(C)(=O)=O)N2C(=O)c3cccc(N4CCN([C@@H]5CCN(Cc6ccc(Oc7ccc8c([C@@]9(C)CCC(=O)NC9=O)nn(C)c8c7)cc6)CC5(F)F)CC4)c3C2=O)ccc1OC]

4-(4-((R)-3,3-Difluoro-1-(4-((5-fluoro-1-methyl-3-((S)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)isoindoline-1,3-dione B037.

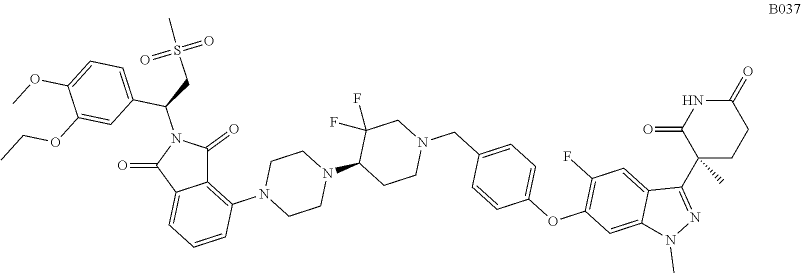 [SMILES: CCOc1cc([C@@H](CS(C)(=O)=O)N2C(=O)c3cccc(N4CCN([C@@H]5CCN(Cc6ccc(Oc7cc8c(cc7F)c([C@]7(C)CCC(=O)NC7=O)nn8C)cc6)CC5(F)F)CC4)c3C2=O)ccc1OC]

4-(4-((R)-3,3-Difluoro-1-(4-((5-fluoro-1-methyl-3-((S)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)isoindoline-1,3-dione B038.

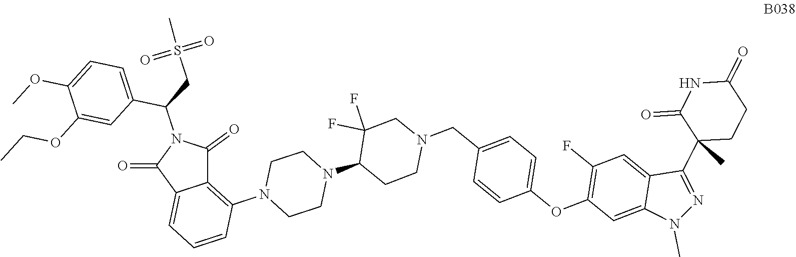 [SMILES: CCOc1cc([C@@H](CS(C)(=O)=O)N2C(=O)c3cccc(N4CCN([C@@H]5CCN(Cc6ccc(Oc7cc8c(cc7F)c([C@@]7(C)CCC(=O)NC7=O)nn8C)cc6)CC5(F)F)CC4)c3C2=O)ccc1OC]

4-(1′-(2-(4-((3-((R)-2,6-Dioxopiperidin-3-yl)-1-methyl-1H-indazol-6-yl)oxy)-phenyl)-2,2-difluoroethyl)-[4,4′-bipiperidin]-1-yl)-2-((S)-1-(3-ethoxy-4-methoxyphenyl)-2-(methylsulfonyl)ethyl)isoindoline-1,3-dione B039.

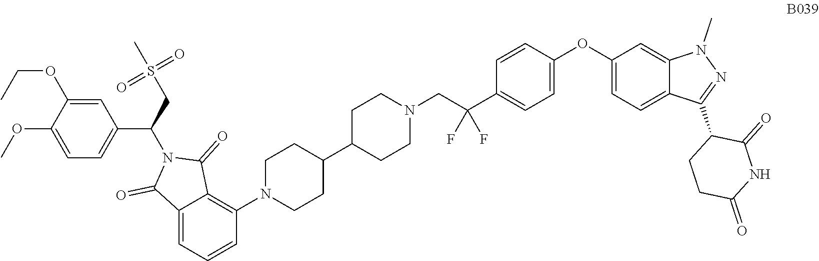 [SMILES: CCOc1cc([C@@H](CS(C)(=O)=O)N2C(=O)c3cccc(N4CCC(C5CCN(CC(F)(F)c6ccc(Oc7ccc8c([C@H]9CCC(=O)NC9=O)nn(C)c8c7)cc6)CC5)CC4)c3C2=O)ccc1OC]

4-(1′-(2-(4-((3-((S)-2,6-Dioxopiperidin-3-yl)-1-methyl-1H-indazol-6-yl)oxy)-phenyl)-2,2-difluoroethyl)-[4,4′-bipiperidin]-1-yl)-2-((S)-1-(3-ethoxy-4-methoxyphenyl)-2-(methylsulfonyl)ethyl)isoindoline-1,3-dione B040.

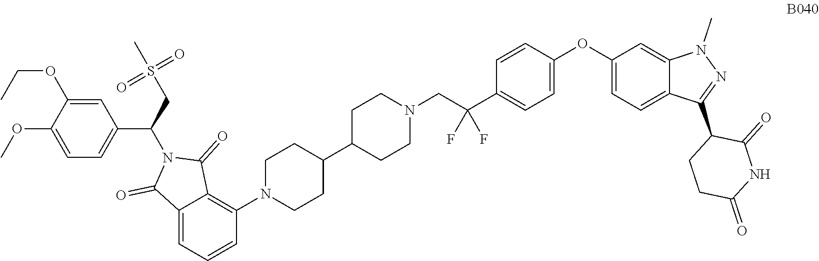 [SMILES: CCOc1cc([C@@H](CS(C)(=O)=O)N2C(=O)c3cccc(N4CCC(C5CCN(CC(F)(F)c6ccc(Oc7ccc8c([C@@H]9CCC(=O)NC9=O)nn(C)c8c7)cc6)CC5)CC4)c3C2=O)ccc1OC]

4-(4-(1-(2,2-Difluoro-2-(4-((1-methyl-3-((S)-3-methyl-2,6-dioxopiperidin-3-yl)-1H-indazol-6-yl)oxy)phenyl)ethyl)piperidin-4-yl)piperazin-1-yl)-2-((S)-1-(3-ethoxy-4-methoxyphenyl)-2-(methylsulfonyl)ethyl)isoindoline-1,3-dione B041.

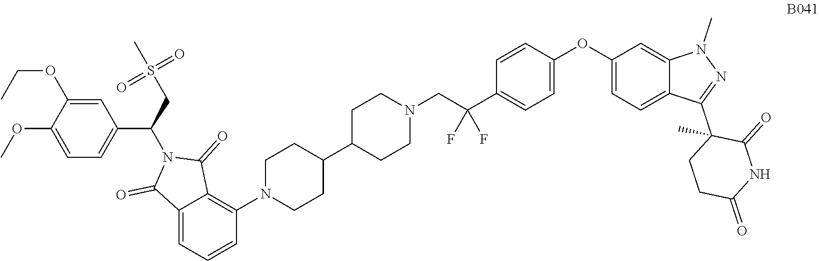 [SMILES: CCOc1cc([C@@H](CS(C)(=O)=O)N2C(=O)c3cccc(N4CCC(C5CCN(CC(F)(F)c6ccc(Oc7ccc8c([C@]9(C)CCC(=O)NC9=O)nn(C)c8c7)cc6)CC5)CC4)c3C2=O)ccc1OC]

4-(4-(1-(2,2-Difluoro-2-(4-((1-methyl-3-((R)-3-methyl-2,6-dioxopiperidin-3-yl)-1H-indazol-6-yl)oxy)phenyl)ethyl)piperidin-4-yl)piperazin-1-yl)-2-((S)-1-(3-ethoxy-4-methoxyphenyl)-2-(methylsulfonyl)ethyl)isoindoline-1,3-dione B042.

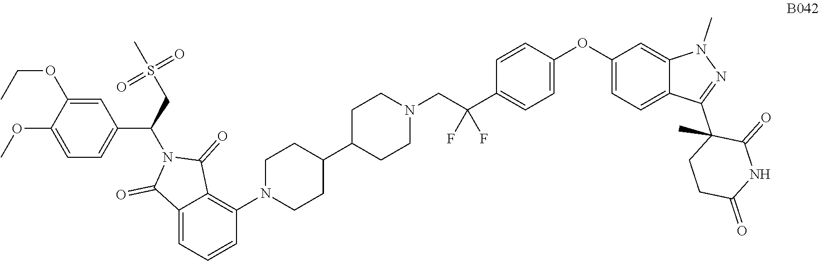 [SMILES: CCOc1cc([C@@H](CS(C)(=O)=O)N2C(=O)c3cccc(N4CCC(C5CCN(CC(F)(F)c6ccc(Oc7ccc8c([C@@]9(C)CCC(=O)NC9=O)nn(C)c8c7)cc6)CC5)CC4)c3C2=O)ccc1OC]

4-(4-(1-(2,2-Difluoro-2-(4-((5-fluoro-1-methyl-3-((S)-3-methyl-2,6-dioxo-piperidin-3-yl)-1H-indazol-6-yl)oxy)phenyl)ethyl)piperidin-4-yl)piperazin-1-yl)-2-((S)-1-(3-ethoxy-4-methoxyphenyl)-2-(methylsulfonyl)ethyl)isoindoline-1,3-dione B043.

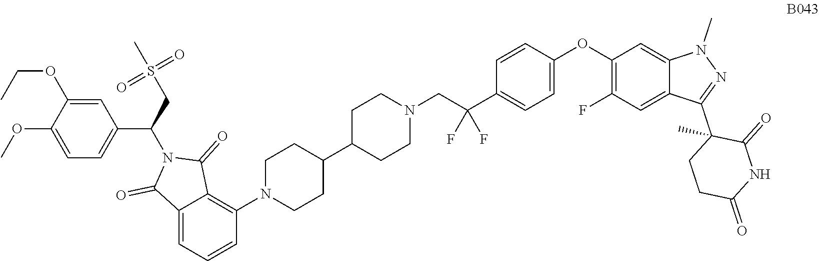 [SMILES: CCOc1cc([C@@H](CS(C)(=O)=O)N2C(=O)c3cccc(N4CCC(C5CCN(CC(F)(F)c6ccc(Oc7cc8c(cc7F)c([C@]7(C)CCC(=O)NC7=O)nn8C)cc6)CC5)CC4)c3C2=O)ccc1OC]

4-(4-(1-(2,2-Difluoro-2-(4-((5-fluoro-1-methyl-3-((R)-3-methyl-2,6-dioxo-piperidin-3-yl)-1H-indazol-6-yl)oxy)phenyl)ethyl)piperidin-4-yl)piperazin-1-yl)-2-((S)-1-(3-ethoxy-4-methoxyphenyl)-2-(methylsulfonyl)ethyl)isoindoline-1,3-dione B044.

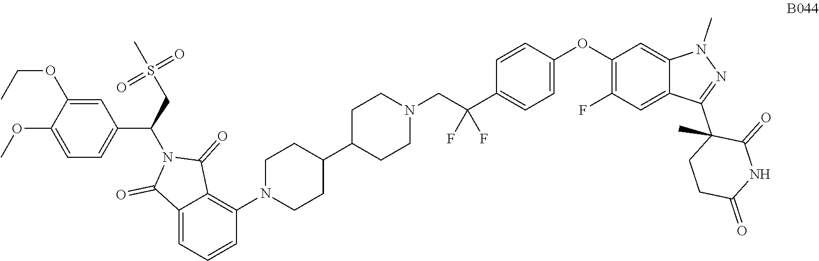 [SMILES: CCOc1cc([C@@H](CS(C)(=O)=O)N2C(=O)c3cccc(N4CCC(C5CCN(CC(F)(F)c6ccc(Oc7cc8c(cc7F)c([C@@]7(C)CCC(=O)NC7=O)nn8C)cc6)CC5)CC4)c3C2=O)ccc1OC]

4-(4-(2-(4-(4-((3-((S)-2,6-Dioxopiperidin-3-yl)-1-methyl-1H-indazol-6-yl)oxy)-phenyl)piperazin-1-yl)ethyl)piperidin-1-yl)-2-((S)-1-(3-ethoxy-4-methoxyphenyl)-2-(methyl-sulfonyl)ethyl)isoindoline-1,3-dione B045.

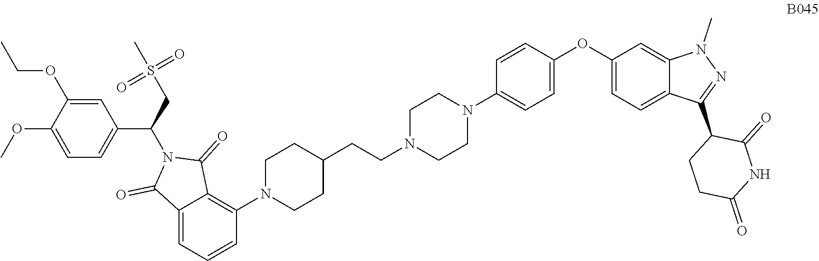 [SMILES: CCOc1cc([C@@H](CS(C)(=O)=O)N2C(=O)c3cccc(N4CCC(CCN5CCN(c6ccc(Oc7ccc8c([C@@H]9CCC(=O)NC9=O)nn(C)c8c7)cc6)CC5)CC4)c3C2=O)ccc1OC]

4-(4-(2-(4-(4-((3-((R)-2,6-Dioxopiperidin-3-yl)-1-methyl-1H-indazol-6-yl)oxy)-phenyl)piperazin-1-yl)ethyl)piperidin-1-yl)-2-((S)-1-(3-ethoxy-4-methoxyphenyl)-2-(methyl-sulfonyl)ethyl)isoindoline-1,3-dione B046.

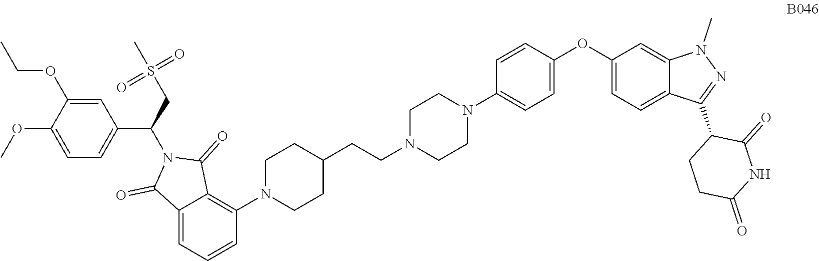 [SMILES: CCOc1cc([C@@H](CS(C)(=O)=O)N2C(=O)c3cccc(N4CCC(CCN5CCN(c6ccc(Oc7ccc8c([C@H]9CCC(=O)NC9=O)nn(C)c8c7)cc6)CC5)CC4)c3C2=O)ccc1OC]

2-((S)-1-(3-Ethoxy-4-methoxyphenyl)-2-(methylsulfonyl)ethyl)-4-(4-(2-(1-(4-((1-methyl-3-((R)-3-methyl-2,6-dioxopiperidin-3-yl)-1H-indazol-6-yl)oxy)phenyl)piperidin-4-yl)-ethyl)piperazin-1-yl)isoindoline-1,3-dione B047.

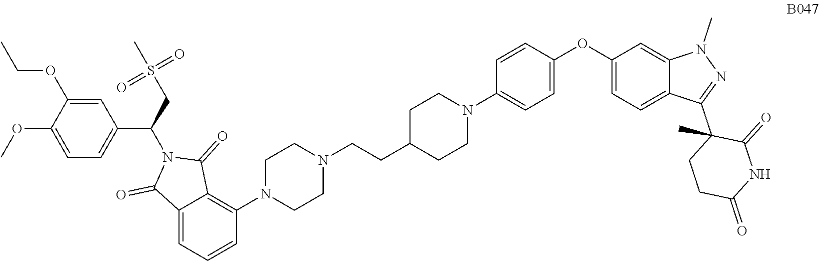 [SMILES: CCOc1cc([C@@H](CS(C)(=O)=O)N2C(=O)c3cccc(N4CCN(CCC5CCN(c6ccc(Oc7ccc8c([C@@]9(C)CCC(=O)NC9=O)nn(C)c8c7)cc6)CC5)CC4)c3C2=O)ccc1OC]

2-((S)-1-(3-Ethoxy-4-methoxyphenyl)-2-(methylsulfonyl)ethyl)-4-(4-(2-(1-(4-((5-fluoro-1-methyl-3-((R)-3-methyl-2,6-dioxopiperidin-3-yl)-1H-indazol-6-yl)oxy)phenyl)-piperidin-4-yl)ethyl)piperazin-1-yl)isoindoline-1,3-dione B048.

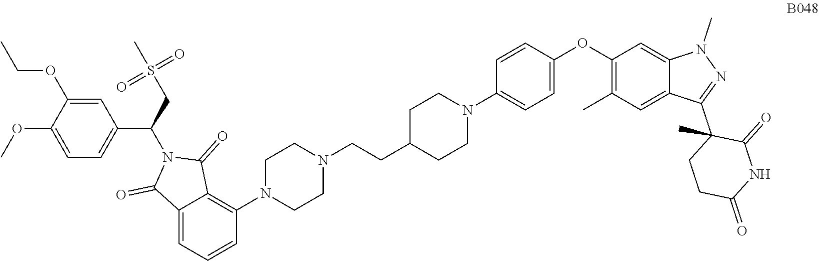 [SMILES: CCOc1cc([C@@H](CS(C)(=O)=O)N2C(=O)c3cccc(N4CCN(CCC5CCN(c6ccc(Oc7cc8c(cc7C)c([C@@]7(C)CCC(=O)NC7=O)nn8C)cc6)CC5)CC4)c3C2=O)ccc1OC]

4-((4'S)-1′-(4-((3-(2,6-Dioxopiperidin-3-yl)-1-methyl-1H-indazol-6-yl)oxy)phen-ethyl)-3′,3′-difluoro-[4,4′-bipiperidin]-1-yl)-2-((S)-1-(3-ethoxy-4-methoxyphenyl)-2-(methyl-sulfonyl)ethyl)isoindoline-1,3-dione B049.

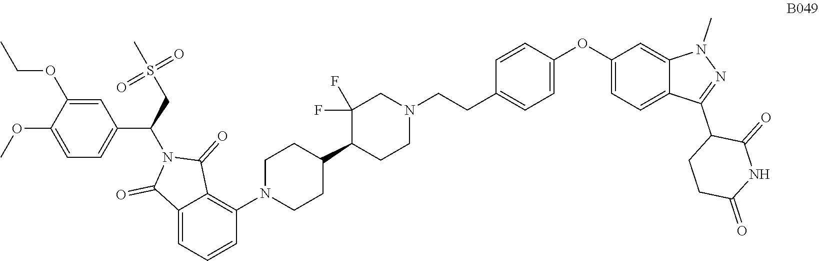 [SMILES: CCOc1cc([C@@H](CS(C)(=O)=O)N2C(=O)c3cccc(N4CCC([C@@H]5CCN(CCc6ccc(Oc7ccc8c(C9CCC(=O)NC9=O)nn(C)c8c7)cc6)CC5(F)F)CC4)c3C2=O)ccc1OC]

6-(4-((S)-3,3-Difluoro-1-(4-((1-methyl-3-((R)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B051.

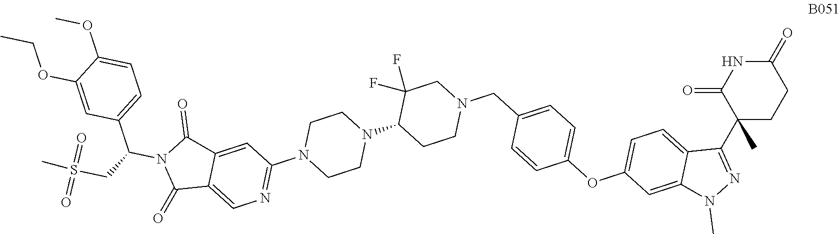 [SMILES: CCOc1cc([C@@H](CS(C)(=O)=O)N2C(=O)c3cnc(N4CCN([C@H]5CCN(Cc6ccc(Oc7ccc8c([C@@]9(C)CCC(=O)NC9=O)nn(C)c8c7)cc6)CC5(F)F)CC4)cc3C2=O)ccc1OC]

6-(4-((S)-3,3-Difluoro-1-(4-((5-fluoro-1-methyl-3-((R)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B052.

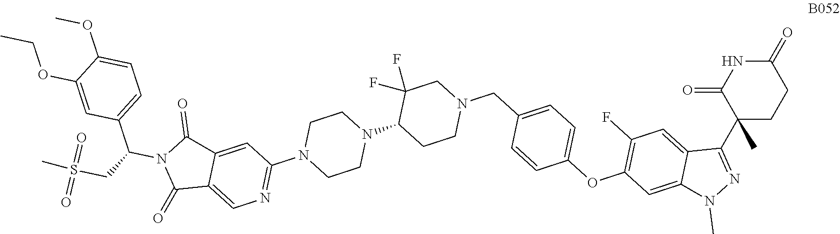 [SMILES: CCOc1cc([C@@H](CS(C)(=O)=O)N2C(=O)c3cnc(N4CCN([C@H]5CCN(Cc6ccc(Oc7cc8c(cc7F)c([C@@]7(C)CCC(=O)NC7=O)nn8C)cc6)CC5(F)F)CC4)cc3C2=O)ccc1OC]

6-(4-((R)-3,3-Difluoro-1-(4-((1-methyl-3-((R)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B053.

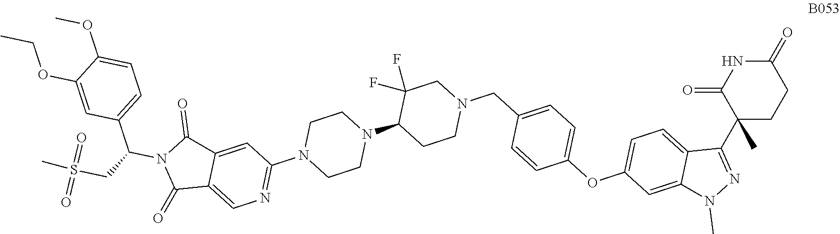 [SMILES: CCOc1cc([C@@H](CS(C)(=O)=O)N2C(=O)c3cnc(N4CCN([C@@H]5CCN(Cc6ccc(Oc7ccc8c([C@@]9(C)CCC(=O)NC9=O)nn(C)c8c7)cc6)CC5(F)F)CC4)cc3C2=O)ccc1OC]

6-(4-((R)-3,3-Difluoro-1-(4-((5-fluoro-1-methyl-3-((R)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B054.

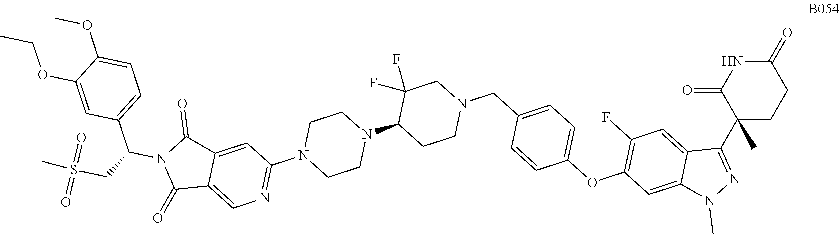 [SMILES: CCOc1cc([C@@H](CS(C)(=O)=O)N2C(=O)c3cnc(N4CCN([C@@H]5CCN(Cc6ccc(Oc7cc8c(cc7F)c([C@@]7(C)CCC(=O)NC7=O)nn8C)cc6)CC5(F)F)CC4)cc3C2=O)ccc1OC]

4-(4-((S)-3,3-Difluoro-1-(4-((1-methyl-3-((R)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B055.

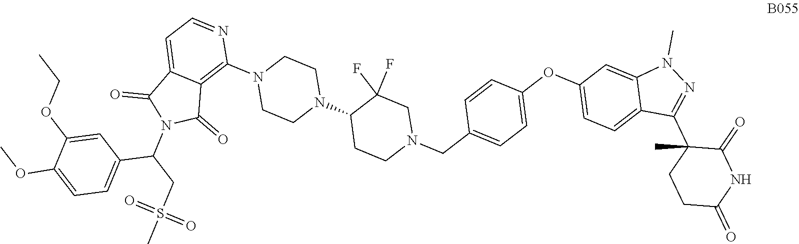 [SMILES: CCOc1cc(C(CS(C)(=O)=O)N2C(=O)c3ccnc(N4CCN([C@H]5CCN(Cc6ccc(Oc7ccc8c([C@@]9(C)CCC(=O)NC9=O)nn(C)c8c7)cc6)CC5(F)F)CC4)c3C2=O)ccc1OC]

4-(4-((S)-3,3-Difluoro-1-(4-((5-fluoro-1-methyl-3-((R)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B056.

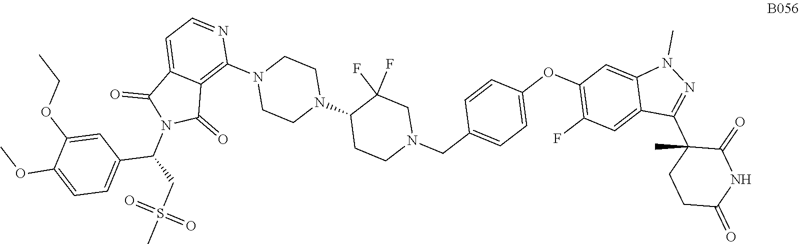 [SMILES: CCOc1cc([C@@H](CS(C)(=O)=O)N2C(=O)c3ccnc(N4CCN([C@H]5CCN(Cc6ccc(Oc7cc8c(cc7F)c([C@@]7(C)CCC(=O)NC7=O)nn8C)cc6)CC5(F)F)CC4)c3C2=O)ccc1OC]

4-(4-((R)-3,3-Difluoro-1-(4-((1-methyl-3-((R)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B057.

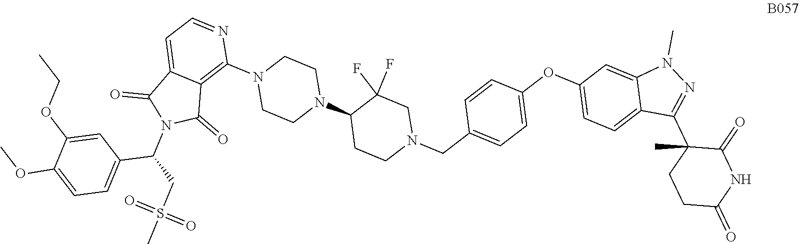 [SMILES: CCOc1cc([C@@H](CS(C)(=O)=O)N2C(=O)c3ccnc(N4CCN([C@@H]5CCN(Cc6ccc(Oc7ccc8c([C@@]9(C)CCC(=O)NC9=O)nn(C)c8c7)cc6)CC5(F)F)CC4)c3C2=O)ccc1OC]

4-(4-((R)-3,3-Difluoro-1-(4-((5-fluoro-1-methyl-3-((R)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B058.

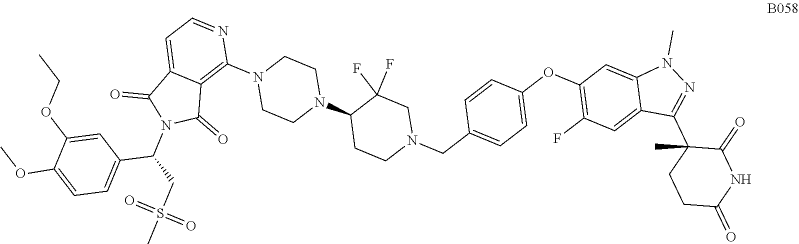 [SMILES: CCOc1cc([C@@H](CS(C)(=O)=O)N2C(=O)c3ccnc(N4CCN([C@@H]5CCN(Cc6ccc(Oc7cc8c(cc7F)c([C@@]7(C)CCC(=O)NC7=O)nn8C)cc6)CC5(F)F)CC4)c3C2=O)ccc1OC]

4-(4-(1-(2,2-Difluoro-2-(4-((1-methyl-3-((S)-3-methyl-2,6-dioxopiperidin-3-yl)-1H-indazol-6-yl)oxy)phenyl)ethyl)piperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B059.

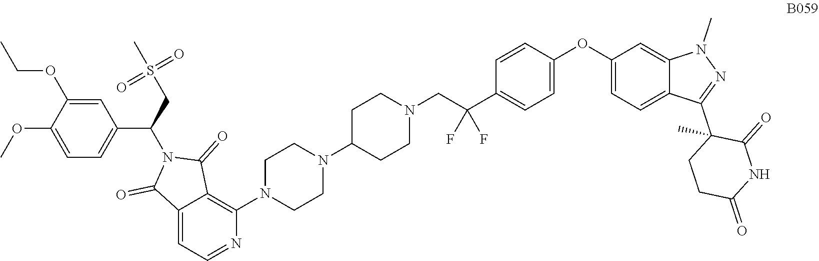 [SMILES: CCOc1cc([C@@H](CS(C)(=O)=O)N2C(=O)c3ccnc(N4CCN(C5CCN(CC(F)(F)c6ccc(Oc7ccc8c([C@]9(C)CCC(=O)NC9=O)nn(C)c8c7)cc6)CC5)CC4)c3C2=O)ccc1OC]

4-(4-(1-(2,2-Difluoro-2-(4-((1-methyl-3-((R)-3-methyl-2,6-dioxopiperidin-3-yl)-1H-indazol-6-yl)oxy)phenyl)ethyl)piperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B060.

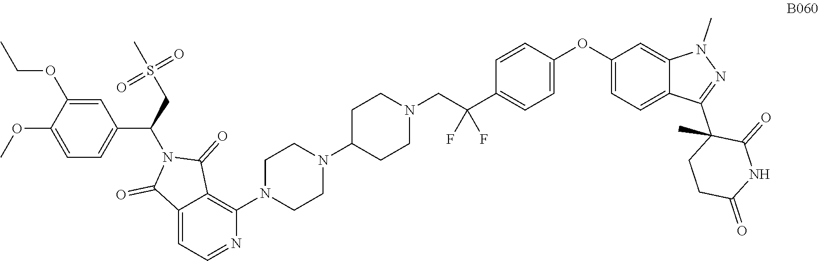 [SMILES: CCOc1cc([C@@H](CS(C)(=O)=O)N2C(=O)c3ccnc(N4CCN(C5CCN(CC(F)(F)c6ccc(Oc7ccc8c([C@@]9(C)CCC(=O)NC9=O)nn(C)c8c7)cc6)CC5)CC4)c3C2=O)ccc1OC]

4-(4-(1-(2,2-Difluoro-2-(4-((5-fluoro-1-methyl-3-((R)-3-methyl-2,6-dioxo-piperidin-3-yl)-1H-indazol-6-yl)oxy)phenyl)ethyl)piperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B061.

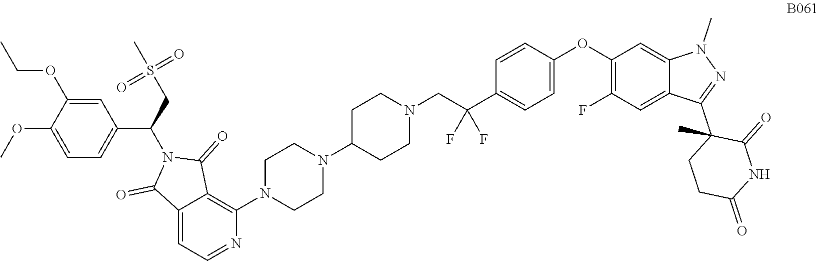 [SMILES: CCOc1cc([C@@H](CS(C)(=O)=O)N2C(=O)c3ccnc(N4CCN(C5CCN(CC(F)(F)c6ccc(Oc7cc8c(cc7F)c([C@@]7(C)CCC(=O)NC7=O)nn8C)cc6)CC5)CC4)c3C2=O)ccc1OC]

5-((R)-1′-(4-((3-(2,4-Dioxotetrahydropyrimidin-1(2H)-yl)-5-fluoro-1-methyl-1H-indazol-6-yl)oxy)benzyl)-3′,3′-difluoro-[4,4′-bipiperidin]-1-yl)-2-((S)-1-(3-ethoxy-4-methoxy-phenyl)-2-(methylsulfonyl)ethyl)isoindoline-1,3-dione B154.

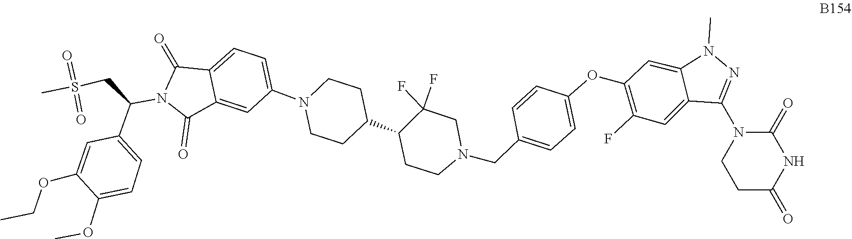 [SMILES: CCOc1cc([C@@H](CS(C)(=O)=O)N2C(=O)c3ccc(N4CCC([C@H]5CCN(Cc6ccc(Oc7cc8c(cc7F)c(N7CCC(=O)NC7=O)nn8C)cc6)CC5(F)F)CC4)cc3C2=O)ccc1OC]

5-(4-((S)-1-(4-((3-(2,4-Dioxotetrahydropyrimidin-1(2H)-yl)-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)isoindoline-1,3-dione B155.

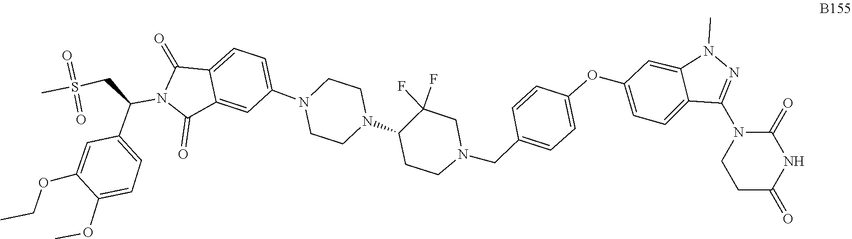 [SMILES: CCOc1cc([C@@H](CS(C)(=O)=O)N2C(=O)c3ccc(N4CCN([C@H]5CCN(Cc6ccc(Oc7ccc8c(N9CCC(=O)NC9=O)nn(C)c8c7)cc6)CC5(F)F)CC4)cc3C2=O)ccc1OC]

5-(4-((R)-1-(4-((3-(2,4-Dioxotetrahydropyrimidin-1(2H)-yl)-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)isoindoline-1,3-dione B156.

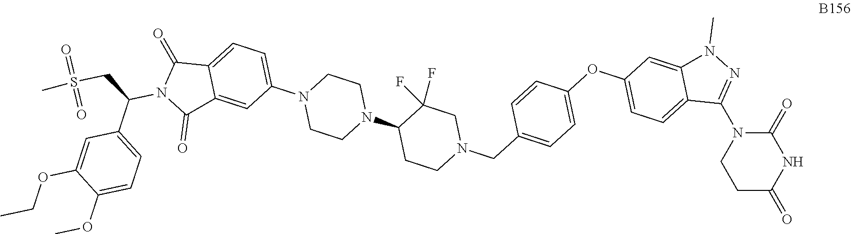 [SMILES: CCOc1cc([C@@H](CS(C)(=O)=O)N2C(=O)c3ccc(N4CCN([C@@H]5CCN(Cc6ccc(Oc7ccc8c(N9CCC(=O)NC9=O)nn(C)c8c7)cc6)CC5(F)F)CC4)cc3C2=O)ccc1OC]

5-(4-((S)-1-(4-((3-(2,4-Dioxotetrahydropyrimidin-1(2H)-yl)-5-fluoro-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)isoindoline-1,3-dione B157.

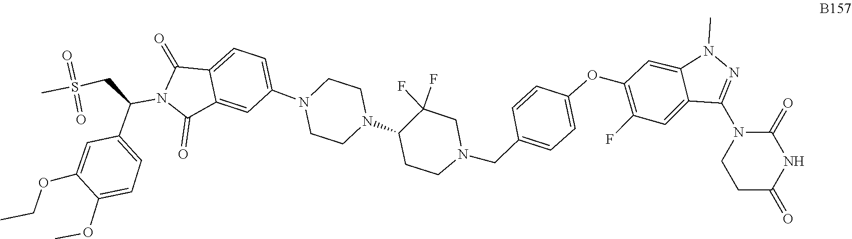 [SMILES: CCOc1cc([C@@H](CS(C)(=O)=O)N2C(=O)c3ccc(N4CCN([C@H]5CCN(Cc6ccc(Oc7cc8c(cc7F)c(N7CCC(=O)NC7=O)nn8C)cc6)CC5(F)F)CC4)cc3C2=O)ccc1OC]

5-(4-((R)-1-(4-((3-(2,4-Dioxotetrahydropyridin-1(2H)-yl)-5-fluoro-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)isoindoline-1,3-dione B158.

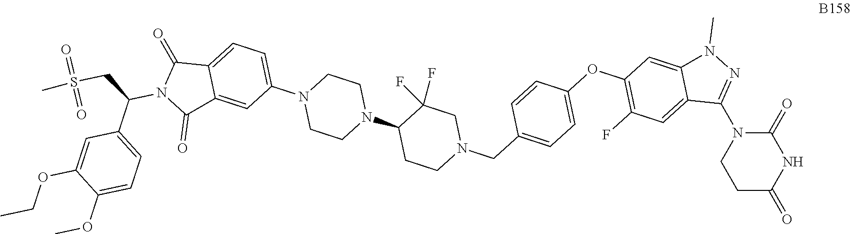 [SMILES: CCOc1cc([C@@H](CS(C)(=O)=O)N2C(=O)c3ccc(N4CCN([C@@H]5CCN(Cc6ccc(Oc7cc8c(cc7F)c(N7CCC(=O)NC7=O)nn8C)cc6)CC5(F)F)CC4)cc3C2=O)ccc1OC]

4-((R)-1′-(4-((3-(2,4-Dioxotetrahydropyrimidin-1(2H)-yl)-1-methyl-1H-indazol-6-yl)oxy)benzyl)-3′,3′-difluoro-[4,4′-bipiperidin]-1-yl)-2-((S)-1-(3-ethoxy-4-methoxyphenyl)-2-(methylsulfonyl)ethyl)isoindoline-1,3-dione B159.

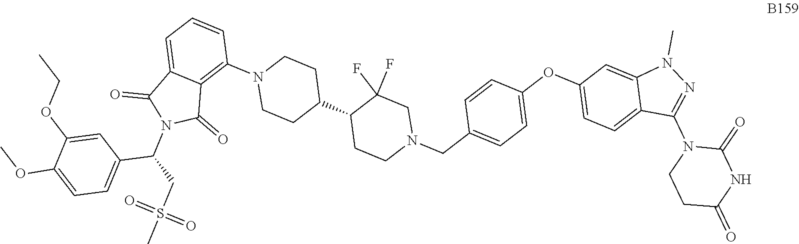 [SMILES: CCOc1cc([C@@H](CS(C)(=O)=O)N2C(=O)c3cccc(N4CCC([C@H]5CCN(Cc6ccc(Oc7ccc8c(N9CCC(=O)NC9=O)nn(C)c8c7)cc6)CC5(F)F)CC4)c3C2=O)ccc1OC]

4-(4-((S)-1-(4-((3-(2,4-Dioxotetrahydropyrimidin-1(2H)-yl)-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)isoindoline-1,3-dione B160.

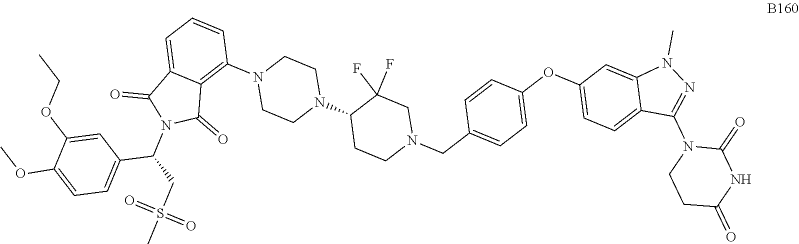 [SMILES: CCOc1cc([C@@H](CS(C)(=O)=O)N2C(=O)c3cccc(N4CCN([C@H]5CCN(Cc6ccc(Oc7ccc8c(N9CCC(=O)NC9=O)nn(C)c8c7)cc6)CC5(F)F)CC4)c3C2=O)ccc1OC]

4-(4-((S)-1-(4-((3-(2,4-Dioxotetrahydropyrimidin-1(2H)-yl)-5-fluoro-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)isoindoline-1,3-dione B161.

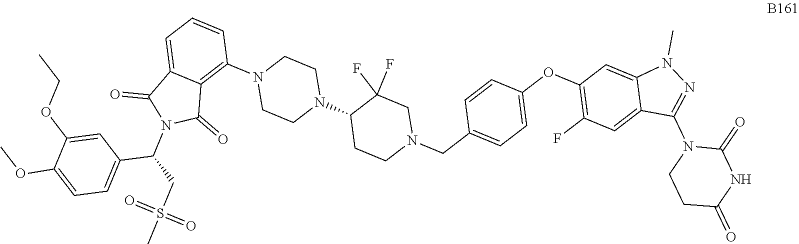 [SMILES: CCOc1cc([C@@H](CS(C)(=O)=O)N2C(=O)c3cccc(N4CCN([C@H]5CCN(Cc6ccc(Oc7cc8c(cc7F)c(N7CCC(=O)NC7=O)nn8C)cc6)CC5(F)F)CC4)c3C2=O)ccc1OC]

4-(4-((R)-1-(4-((3-(2,4-Dioxotetrahydropyrimidin-1(2H)-yl)-5-fluoro-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)isoindoline-1,3-dione B162.

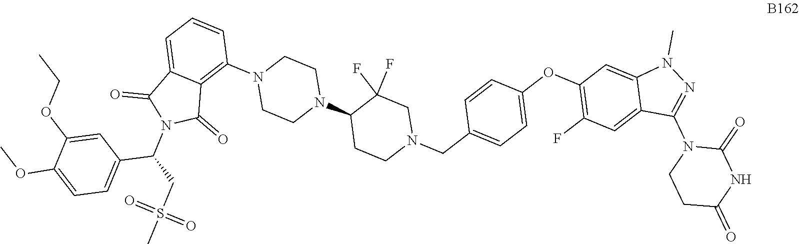 [SMILES: CCOc1cc([C@@H](CS(C)(=O)=O)N2C(=O)c3cccc(N4CCN([C@@H]5CCN(Cc6ccc(Oc7cc8c(cc7F)c(N7CCC(=O)NC7=O)nn8C)cc6)CC5(F)F)CC4)c3C2=O)ccc1OC]

(S)-4-(4-((4-(4-((3-(2,4-Dioxotetrahydropyrimidin-1(2H)-yl)-1-methyl-1H-indazol-6-yl)oxy)phenyl)piperidin-1-yl)methyl)-4-fluoropiperidin-1-yl)-2-(1-(3-ethoxy-4-methoxyphenyl)-2-(methylsulfonyl)ethyl)isoindoline-1,3-dione B163.

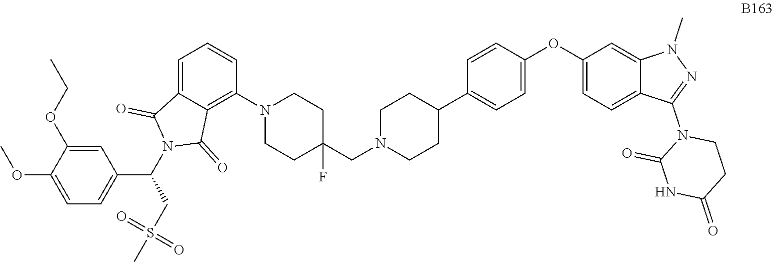 [SMILES: CCOc1cc([C@@H](CS(C)(=O)=O)N2C(=O)c3cccc(N4CCC(F)(CN5CCC(c6ccc(Oc7ccc8c(N9CCC(=O)NC9=O)nn(C)c8c7)cc6)CC5)CC4)c3C2=O)ccc1OC]

(S)-4-(4-((4-(4-((3-(2,4-Dioxotetrahydropyrimidin-1(2H)-yl)-5-fluoro-1-methyl-1H-indazol-6-yl)oxy)phenyl)piperidin-1-yl)methyl)-4-fluoropiperidin-1-yl)-2-(1-(3-ethoxy-4-methoxyphenyl)-2-(methylsulfonyl)ethyl)isoindoline-1,3-dione B164.

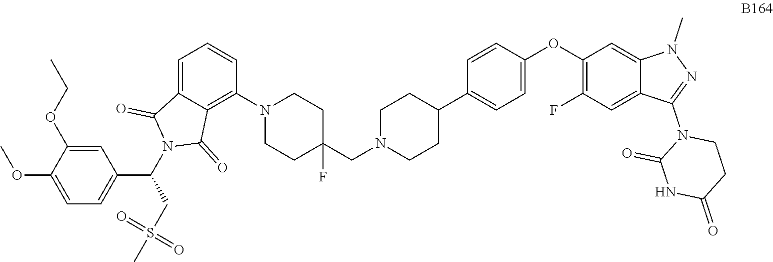 [SMILES: CCOc1cc([C@@H](CS(C)(=O)=O)N2C(=O)c3cccc(N4CCC(F)(CN5CCC(c6ccc(Oc7cc8c(cc7F)c(N7CCC(=O)NC7=O)nn8C)cc6)CC5)CC4)c3C2=O)ccc1OC]

(S)-5-(4-((4-(4-((3-(2,4-Dioxotetrahydropyrimidin-1(2H)-yl)-1-methyl-1H-indazol-6-yl)oxy)phenyl)piperidin-1-yl)methyl)-4-fluoropiperidin-1-yl)-2-(1-(3-ethoxy-4-methoxyphenyl)-2-(methylsulfonyl)ethyl)isoindoline-1,3-dione B165.

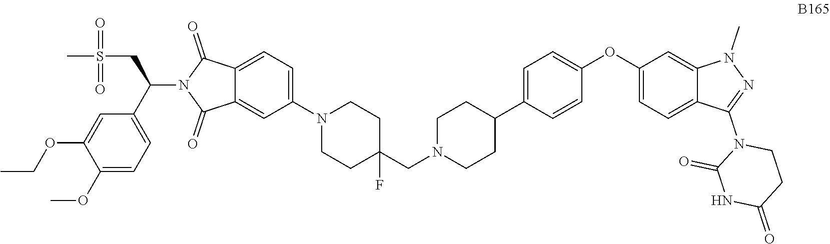 [SMILES: CCOc1cc([C@@H](CS(C)(=O)=O)N2C(=O)c3ccc(N4CCC(F)(CN5CCC(c6ccc(Oc7ccc8c(N9CCC(=O)NC9=O)nn(C)c8c7)cc6)CC5)CC4)cc3C2=O)ccc1OC]

(S)-5-(4-((4-(4-((3-(2,4-Dioxotetrahydropyrimidin-1(2H)-yl)-5-fluoro-1-methyl-1H-indazol-6-yl)oxy)phenyl)piperidin-1-yl)methyl)-4-fluoropiperidin-1-yl)-2-(1-(3-ethoxy-4-methoxyphenyl)-2-(methylsulfonyl)ethyl)isoindoline-1,3-dione B166.

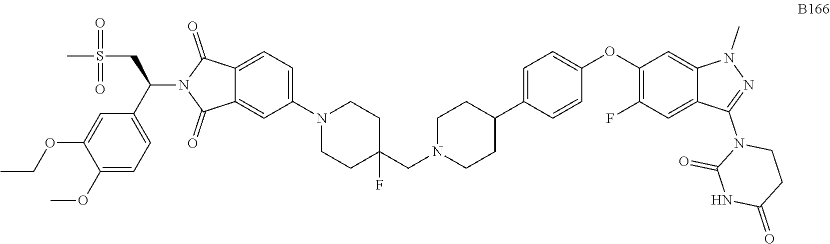 [SMILES: CCOc1cc([C@@H](CS(C)(=O)=O)N2C(=O)c3ccc(N4CCC(F)(CN5CCC(c6ccc(Oc7cc8c(cc7F)c(N7CCC(=O)NC7=O)nn8C)cc6)CC5)CC4)cc3C2=O)ccc1OC]

(S)-4-(1′-(2-(4-((3-(2,4-Dioxotetrahydropyrimidin-1(2H)-yl)-1-methyl-1H-indazol-6-yl)oxy)phenyl)-2,2-difluoroethyl)-[4,4′-bipiperidin]-1-yl)-2-(1-(3-ethoxy-4-methoxy-phenyl)-2-(methylsulfonyl)ethyl)isoindoline-1,3-dione B167.

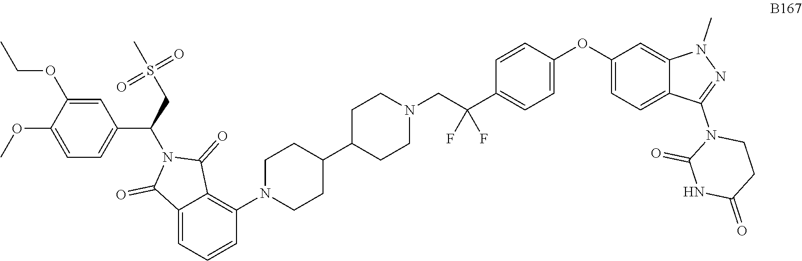 [SMILES: CCOc1cc([C@@H](CS(C)(=O)=O)N2C(=O)c3cccc(N4CCC(C5CCN(CC(F)(F)c6ccc(Oc7ccc8c(N9CCC(=O)NC9=O)nn(C)c8c7)cc6)CC5)CC4)c3C2=O)ccc1OC]

(S)-4-(1′-(2-(4-((3-(2,4-Dioxotetrahydropyrimidin-1(2H)-yl)-5-fluoro-1-methyl-1H-indazol-6-yl)oxy)phenyl)-2,2-difluoroethyl)-[4,4′-bipiperidin]-1-yl)-2-(1-(3-ethoxy-4-methoxyphenyl)-2-(methylsulfonyl)ethyl)isoindoline-1,3-dione B168.

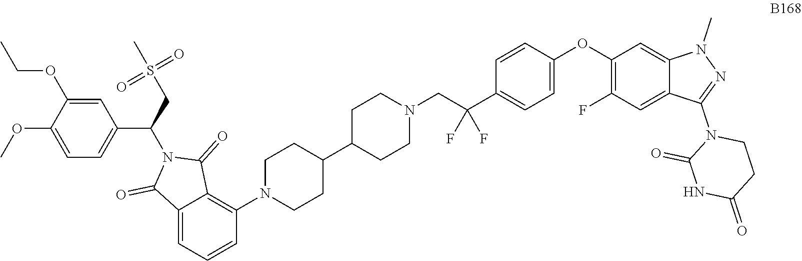 [SMILES: CCOc1cc([C@@H](CS(C)(=O)=O)N2C(=O)c3cccc(N4CCC(C5CCN(CC(F)(F)c6ccc(Oc7cc8c(cc7F)c(N7CCC(=O)NC7=O)nn8C)cc6)CC5)CC4)c3C2=O)ccc1OC]

(S)-4-(4-(1-(2-(4-((3-(2,4-Dioxotetrahydropyrimidin-1(2H)-yl)-1-methyl-1H-indazol-6-yl)oxy)phenyl)-2,2-difluoroethyl)piperidin-4-yl)piperazin-1-yl)-2-(1-(3-ethoxy-4-methoxyphenyl)-2-(methylsulfonyl)ethyl)isoindoline-1,3-dione B169.

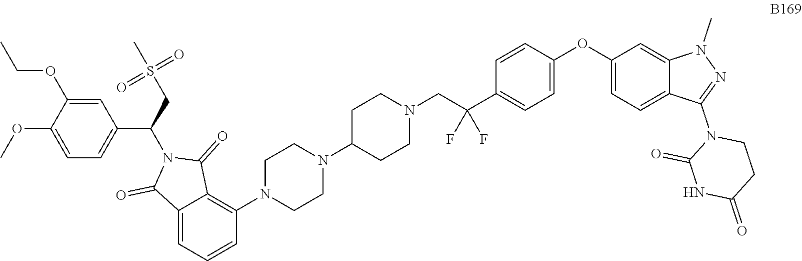 [SMILES: CCOc1cc([C@@H](CS(C)(=O)=O)N2C(=O)c3cccc(N4CCN(C5CCN(CC(F)(F)c6ccc(Oc7ccc8c(N9CCC(=O)NC9=O)nn(C)c8c7)cc6)CC5)CC4)c3C2=O)ccc1OC]

(S)-4-(4-(1-(2-(4-((3-(2,4-Dioxotetrahydropyrimidin-1(2H)-yl)-5-fluoro-1-methyl-1H-indazol-6-yl)oxy)phenyl)-2,2-difluoroethyl)piperidin-4-yl)piperazin-1-yl)-2-(1-(3-ethoxy-4-methoxyphenyl)-2-(methylsulfonyl)ethyl)isoindoline-1,3-dione B170.

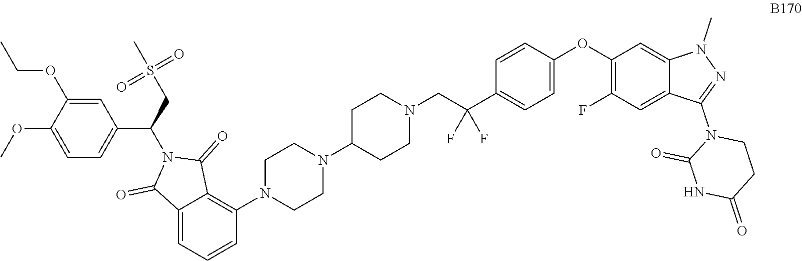 [SMILES: CCOc1cc([C@@H](CS(C)(=O)=O)N2C(=O)c3cccc(N4CCN(C5CCN(CC(F)(F)c6ccc(Oc7cc8c(cc7F)c(N7CCC(=O)NC7=O)nn8C)cc6)CC5)CC4)c3C2=O)ccc1OC]

(S)-4-(4-(2-(4-(4-((3-(2,4-Dioxotetrahydropyrimidin-1(2H)-yl)-1-methyl-1H-indazol-6-yl)oxy)phenyl)piperazin-1-yl)ethyl)piperidin-1-yl)-2-(1-(3-ethoxy-4-methoxyphenyl)-2-(methylsulfonyl)ethyl)isoindoline-1,3-dione B171.

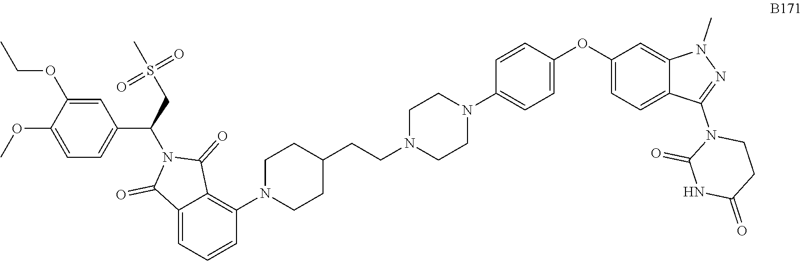 [SMILES: CCOc1cc([C@@H](CS(C)(=O)=O)N2C(=O)c3cccc(N4CCC(CCN5CCN(c6ccc(Oc7ccc8c(N9CCC(=O)NC9=O)nn(C)c8c7)cc6)CC5)CC4)c3C2=O)ccc1OC]

(S)-4-(4-(2-(4-(4-((3-(2,4-Dioxotetrahydropyrimidin-1(2H)-yl)-5-fluoro-1-methyl-1H-indazol-6-yl)oxy)phenyl)piperazin-1-yl)ethyl)piperidin-1-yl)-2-(1-(3-ethoxy-4-methoxyphenyl)-2-(methylsulfonyl)ethyl)isoindoline-1,3-dione B172.

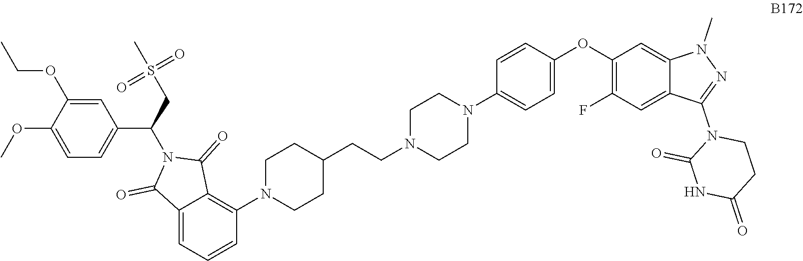 [SMILES: CCOc1cc([C@@H](CS(C)(=O)=O)N2C(=O)c3cccc(N4CCC(CCN5CCN(c6ccc(Oc7cc8c(cc7F)c(N7CCC(=O)NC7=O)nn8C)cc6)CC5)CC4)c3C2=O)ccc1OC]

(S)-4-(4-(2-(1-(4-((3-(2,4-Dioxotetrahydropyrimidin-1(2H)-yl)-1-methyl-1H-indazol-6-yl)oxy)phenyl)piperidin-4-yl)ethyl)piperazin-1-yl)-2-(1-(3-ethoxy-4-methoxyphenyl)-2-(methylsulfonyl)ethyl)isoindoline-1,3-dione B173.

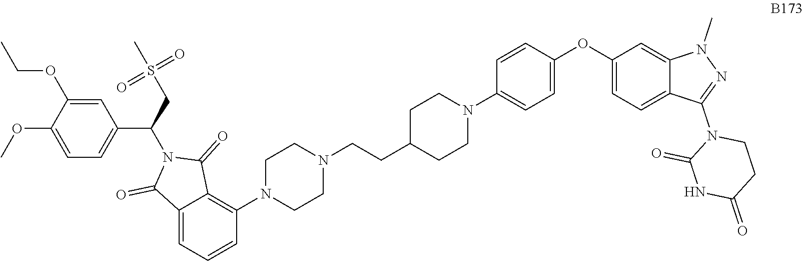 [SMILES: CCOc1cc([C@@H](CS(C)(=O)=O)N2C(=O)c3cccc(N4CCN(CCC5CCN(c6ccc(Oc7ccc8c(N9CCC(=O)NC9=O)nn(C)c8c7)cc6)CC5)CC4)c3C2=O)ccc1OC]

(S)-4-(4-(2-(1-(4-((3-(2,4-Dioxotetrahydropyrimidin-1(2H)-yl)-5-fluoro-1-methyl-1H-indazol-6-yl)oxy)phenyl)piperidin-4-yl)ethyl)piperazin-1-yl)-2-(1-(3-ethoxy-4-methoxyphenyl)-2-(methylsulfonyl)ethyl)isoindoline-1,3-dione B174.

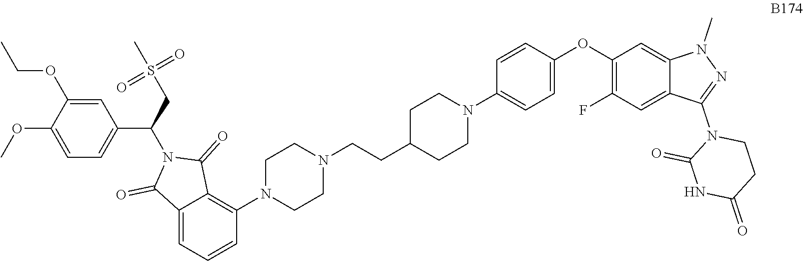 [SMILES: CCOc1cc([C@@H](CS(C)(=O)=O)N2C(=O)c3cccc(N4CCN(CCC5CCN(c6ccc(Oc7cc8c(cc7F)c(N7CCC(=O)NC7=O)nn8C)cc6)CC5)CC4)c3C2=O)ccc1OC]

4-((S)-1′-(4-((3-(2,4-Dioxotetrahydropyrimidin-1(2H)-yl)-1-methyl-1H-indazol-6-yl)oxy)phenethyl)-3′,3′-difluoro-[4,4′-bipiperidin]-1-yl)-2-((S)-1-(3-ethoxy-4-methoxyphenyl)-2-(methylsulfonyl)ethyl)isoindoline-1,3-dione B175.

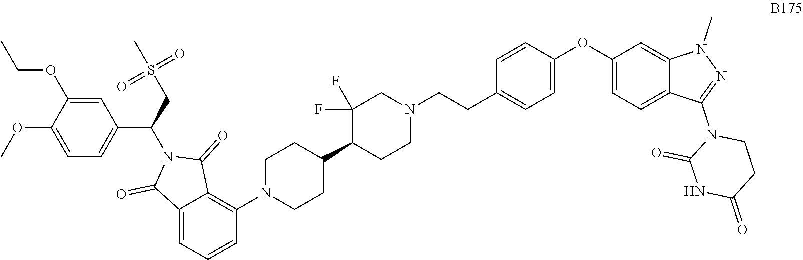 [SMILES: CCOc1cc([C@@H](CS(C)(=O)=O)N2C(=O)c3cccc(N4CCC([C@@H]5CCN(CCc6ccc(Oc7ccc8c(N9CCC(=O)NC9=O)nn(C)c8c7)cc6)CC5(F)F)CC4)c3C2=O)ccc1OC]

4-((S)-1′-(4-((3-(2,4-Dioxotetrahydropyrimidin-1(2H)-yl)-5-fluoro-1-methyl-1H-indazol-6-yl)oxy)phenethyl)-3′,3′-difluoro-[4,4′-bipiperidin]-1-yl)-2-((S)-1-(3-ethoxy-4-methoxy-phenyl)-2-(methylsulfonyl)ethyl)isoindoline-1,3-dione B176.

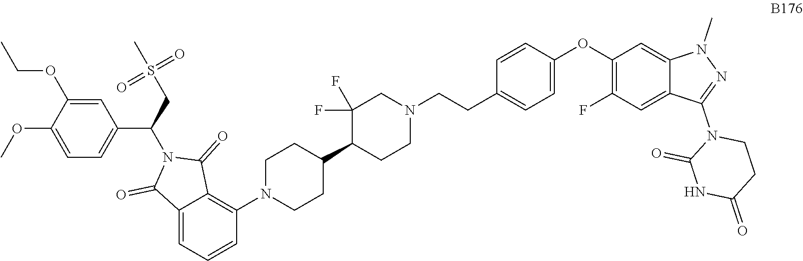 [SMILES: CCOc1cc([C@@H](CS(C)(=O)=O)N2C(=O)c3cccc(N4CCC([C@@H]5CCN(CCc6ccc(Oc7cc8c(cc7F)c(N7CCC(=O)NC7=O)nn8C)cc6)CC5(F)F)CC4)c3C2=O)ccc1OC]

4-(4-((R)-1-(4-((3-(2,4-Dioxotetrahydropyrimidin-1(2H)-yl)-1-methyl-1H-indazol-6-yl)oxy)phenethyl)-3,3-difluoropiperidin-4-yl)piperazin-1-yl)-2-((S)-1-(3-ethoxy-4-methoxyphenyl)-2-(methylsulfonyl)ethyl)isoindoline-1,3-dione B177.

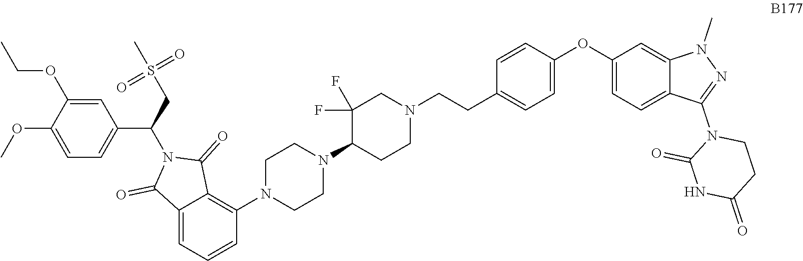 [SMILES: CCOc1cc([C@@H](CS(C)(=O)=O)N2C(=O)c3cccc(N4CCN([C@@H]5CCN(CCc6ccc(Oc7ccc8c(N9CCC(=O)NC9=O)nn(C)c8c7)cc6)CC5(F)F)CC4)c3C2=O)ccc1OC]

4-(4-((R)-1-(4-((3-(2,4-Dioxotetrahydropyrimidin-1(2H)-yl)-5-fluoro-1-methyl-1H-indazol-6-yl)oxy)phenethyl)-3,3-difluoropiperidin-4-yl)piperazin-1-yl)-2-((S)-1-(3-ethoxy-4-methoxyphenyl)-2-(methylsulfonyl)ethyl)isoindoline-1,3-dione B178.

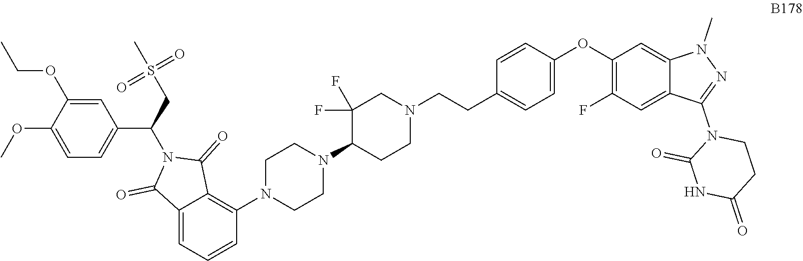 [SMILES: CCOc1cc([C@@H](CS(C)(=O)=O)N2C(=O)c3cccc(N4CCN([C@@H]5CCN(CCc6ccc(Oc7cc8c(cc7F)c(N7CCC(=O)NC7=O)nn8C)cc6)CC5(F)F)CC4)c3C2=O)ccc1OC]

4-(4-((S)-1-(4-((3-(2,4-Dioxotetrahydropyrimidin-1(2H)-yl)-1-methyl-1H-indazol-6-yl)oxy)phenethyl)-3,3-difluoropiperidin-4-yl)piperazin-1-yl)-2-((S)-1-(3-ethoxy-4-methoxyphenyl)-2-(methylsulfonyl)ethyl)isoindoline-1,3-dione B179.

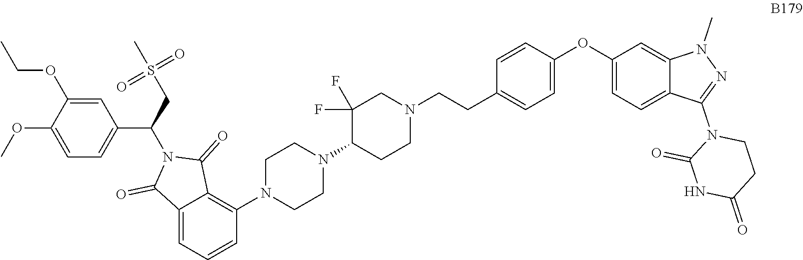 [SMILES: CCOc1cc([C@@H](CS(C)(=O)=O)N2C(=O)c3cccc(N4CCN([C@H]5CCN(CCc6ccc(Oc7ccc8c(N9CCC(=O)NC9=O)nn(C)c8c7)cc6)CC5(F)F)CC4)c3C2=O)ccc1OC]

4-(4-((S)-1-(4-((3-(2,4-Dioxotetrahydropyrimidin-1(2H)-yl)-5-fluoro-1-methyl-1H-indazol-6-yl)oxy)phenethyl)-3,3-difluoropiperidin-4-yl)piperazin-1-yl)-2-((S)-1-(3-ethoxy-4-methoxyphenyl)-2-(methylsulfonyl)ethyl)isoindoline-1,3-dione B180.

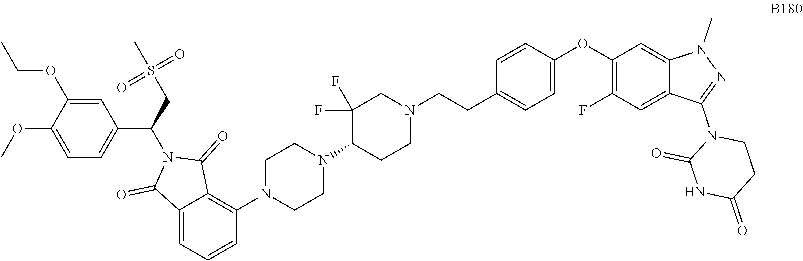 [SMILES: CCOc1cc([C@@H](CS(C)(=O)=O)N2C(=O)c3cccc(N4CCN([C@H]5CCN(CCc6ccc(Oc7cc8c(cc7F)c(N7CCC(=O)NC7=O)nn8C)cc6)CC5(F)F)CC4)c3C2=O)ccc1OC]

6-((R)-1′-(4-((3-(2,4-Dioxotetrahydropyrimidin-1(2H)-yl)-1-methyl-1H-indazol-6-yl)oxy)benzyl)-3′,3′-difluoro-[4,4′-bipiperidin]-1-yl)-2-((S)-1-(3-ethoxy-4-methoxyphenyl)-2-(methylsulfonyl)ethyl)-1H-pyrrolo[3,4-c]pyridine-1,3(2H)-dione B201.

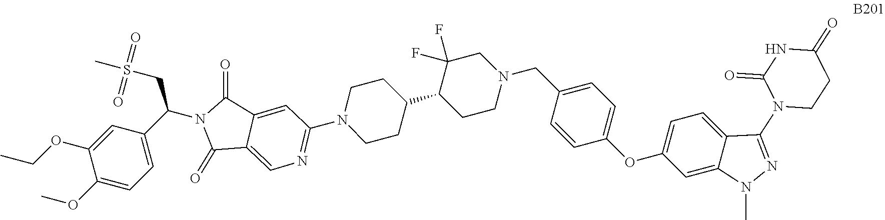 [SMILES: CCOc1cc([C@@H](CS(C)(=O)=O)N2C(=O)c3cnc(N4CCC([C@H]5CCN(Cc6ccc(Oc7ccc8c(N9CCC(=O)NC9=O)nn(C)c8c7)cc6)CC5(F)F)CC4)cc3C2=O)ccc1OC]

6-((R)-1′-(4-((3-(2,4-Dioxotetrahydropyrimidin-1(2H)-yl)-5-fluoro-1-methyl-1H-indazol-6-yl)oxy)benzyl)-3′,3′-difluoro-[4,4′-bipiperidin]-1-yl)-2-((S)-1-(3-ethoxy-4-methoxy-phenyl)-2-(methylsulfonyl)ethyl)-1H-pyrrolo[3,4-c]pyridine-1,3(2H)-dione B202.

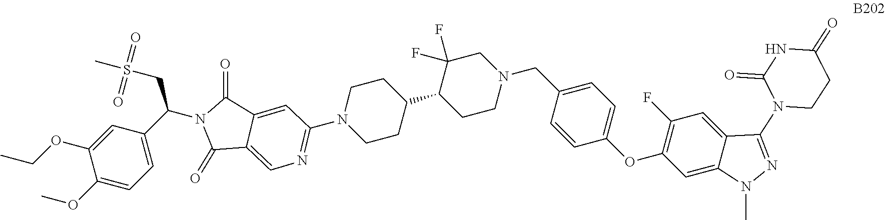 [SMILES: CCOc1cc([C@@H](CS(C)(=O)=O)N2C(=O)c3cnc(N4CCC([C@H]5CCN(Cc6ccc(Oc7cc8c(cc7F)c(N7CCC(=O)NC7=O)nn8C)cc6)CC5(F)F)CC4)cc3C2=O)ccc1OC]

6-(4-((S)-1-(4-((3-(2,4-Dioxotetrahydropyrimidin-1(2H)-yl)-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B203.

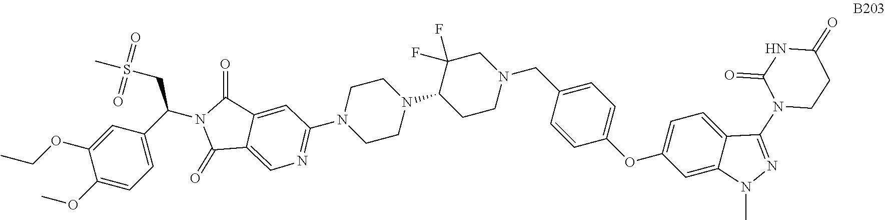 [SMILES: CCOc1cc([C@@H](CS(C)(=O)=O)N2C(=O)c3cnc(N4CCN([C@H]5CCN(Cc6ccc(Oc7ccc8c(N9CCC(=O)NC9=O)nn(C)c8c7)cc6)CC5(F)F)CC4)cc3C2=O)ccc1OC]

6-(4-((S)-1-(4-((3-(2,4-Dioxotetrahydropyrimidin-1(2H)-yl)-5-fluoro-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B204.

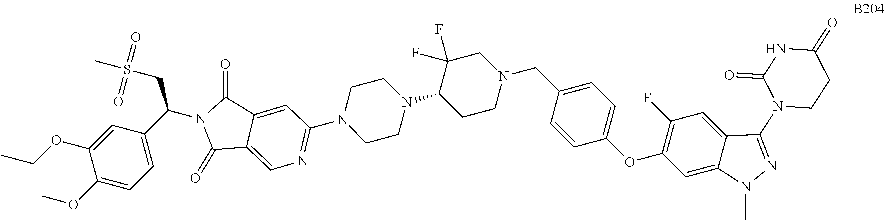 [SMILES: CCOc1cc([C@@H](CS(C)(=O)=O)N2C(=O)c3cnc(N4CCN([C@H]5CCN(Cc6ccc(Oc7cc8c(cc7F)c(N7CCC(=O)NC7=O)nn8C)cc6)CC5(F)F)CC4)cc3C2=O)ccc1OC]

6-(4-((R)-1-(4-((3-(2,4-Dioxotetrahydropyrimidin-1(2H)-yl)-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B205.

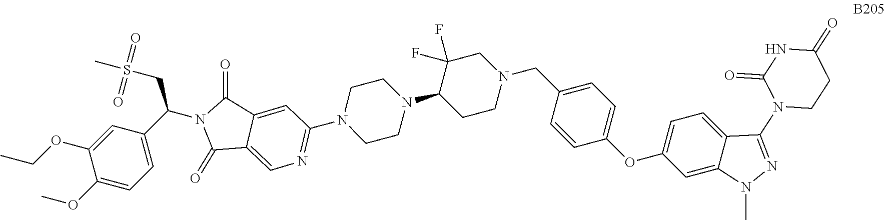 [SMILES: CCOc1cc([C@@H](CS(C)(=O)=O)N2C(=O)c3cnc(N4CCN([C@@H]5CCN(Cc6ccc(Oc7ccc8c(N9CCC(=O)NC9=O)nn(C)c8c7)cc6)CC5(F)F)CC4)cc3C2=O)ccc1OC]

6-(4-((R)-1-(4-((3-(2,4-Dioxotetrahydropyrimidin-1(2H)-yl)-5-fluoro-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B206.

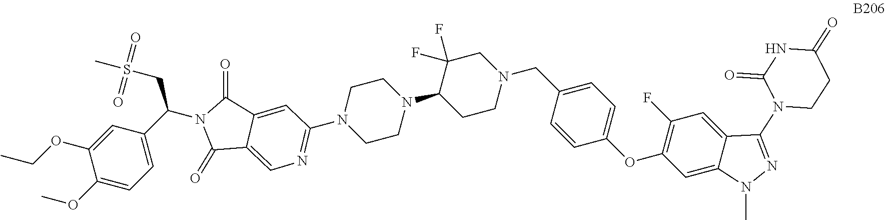 [SMILES: CCOc1cc([C@@H](CS(C)(=O)=O)N2C(=O)c3cnc(N4CCN([C@@H]5CCN(Cc6ccc(Oc7cc8c(cc7F)c(N7CCC(=O)NC7=O)nn8C)cc6)CC5(F)F)CC4)cc3C2=O)ccc1OC]

4-((R)-1′-(4-((3-(2,4-Dioxotetrahydropyrimidin-1(2H)-yl)-1-methyl-1H-indazol-6-yl)oxy)benzyl)-3′,3′-difluoro-[4,4′-bipiperidin]-1-yl)-2-((S)-1-(3-ethoxy-4-methoxyphenyl)-2-(methylsulfonyl)ethyl)-1H-pyrrolo[3,4-c]pyridine-1,3(2H)-dione B207.

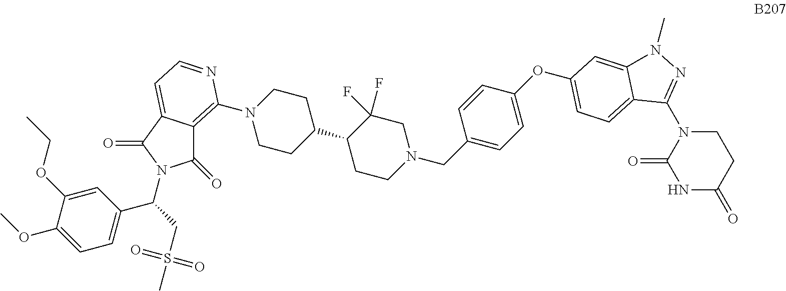 [SMILES: CCOc1cc([C@@H](CS(C)(=O)=O)N2C(=O)c3ccnc(N4CCC([C@H]5CCN(Cc6ccc(Oc7ccc8c(N9CCC(=O)NC9=O)nn(C)c8c7)cc6)CC5(F)F)CC4)c3C2=O)ccc1OC]

4-((R)-1′-(4-((3-(2,4-Dioxotetrahydropyrimidin-1(2H)-yl)-5-fluoro-1-methyl-1H-indazol-6-yl)oxy)benzyl)-3′,3′-difluoro-[4,4′-bipiperidin]-1-yl)-2-((S)-1-(3-ethoxy-4-methoxy-phenyl)-2-(methylsulfonyl)ethyl)-1H-pyrrolo[3,4-c]pyridine-1,3(2H)-dione B208.

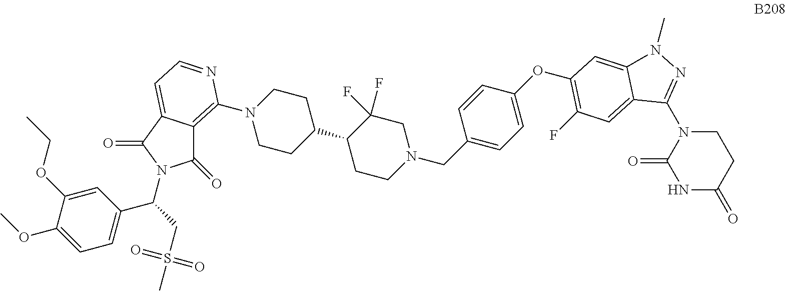 [SMILES: CCOc1cc([C@@H](CS(C)(=O)=O)N2C(=O)c3ccnc(N4CCC([C@H]5CCN(Cc6ccc(Oc7cc8c(cc7F)c(N7CCC(=O)NC7=O)nn8C)cc6)CC5(F)F)CC4)c3C2=O)ccc1OC]

4-(4-((S)-1-(4-((3-(2,4-Dioxotetrahydropyrimidin-1(2H)-yl)-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(21)-dione B209.

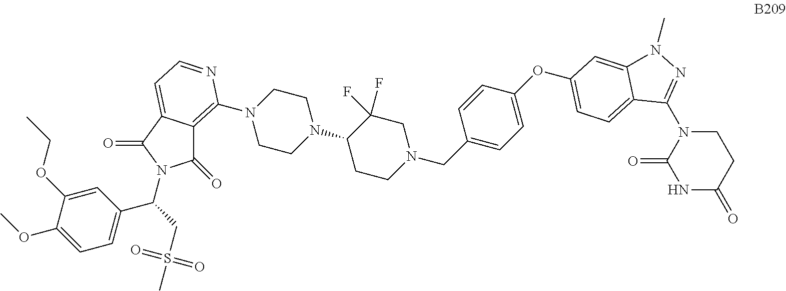 [SMILES: CCOc1cc([C@@H](CS(C)(=O)=O)N2C(=O)c3ccnc(N4CCN([C@H]5CCN(Cc6ccc(Oc7ccc8c(N9CCC(=O)NC9=O)nn(C)c8c7)cc6)CC5(F)F)CC4)c3C2=O)ccc1OC]

4-(4-((S)-1-(4-((3-(2,4-Dioxotetrahydropyrimidin-1(2H)-yl)-5-fluoro-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B210.

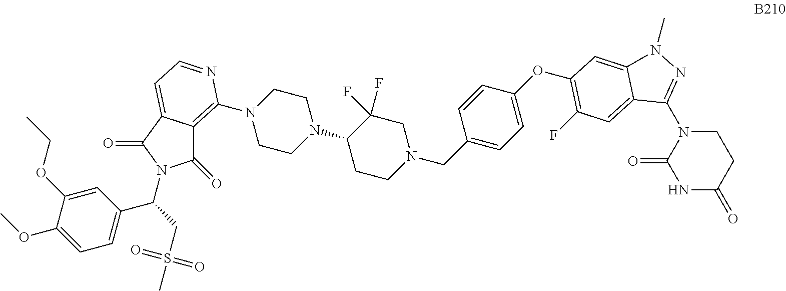 [SMILES: CCOc1cc([C@@H](CS(C)(=O)=O)N2C(=O)c3ccnc(N4CCN([C@H]5CCN(Cc6ccc(Oc7cc8c(cc7F)c(N7CCC(=O)NC7=O)nn8C)cc6)CC5(F)F)CC4)c3C2=O)ccc1OC]

4-(4-((R)-1-(4-((3-(2,4-Dioxotetrahydropyrimidin-1(2H)-yl)-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B211.

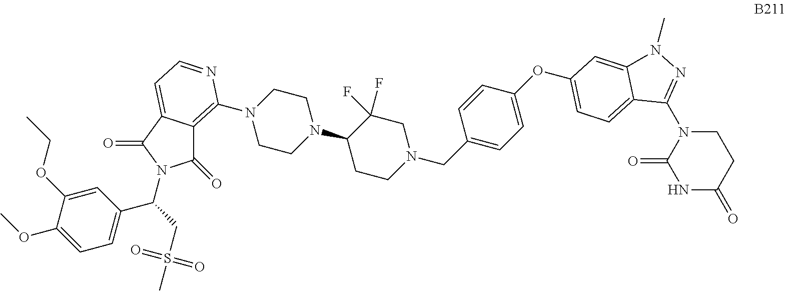 [SMILES: CCOc1cc([C@@H](CS(C)(=O)=O)N2C(=O)c3ccnc(N4CCN([C@@H]5CCN(Cc6ccc(Oc7ccc8c(N9CCC(=O)NC9=O)nn(C)c8c7)cc6)CC5(F)F)CC4)c3C2=O)ccc1OC]

4-(4-((R)-1-(4-((3-(2,4-Dioxotetrahydropyrimidin-1(2H)-yl)-5-fluoro-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B212.

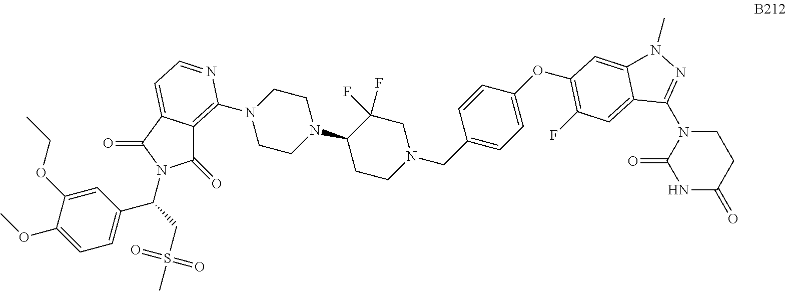 [SMILES: CCOc1cc([C@@H](CS(C)(=O)=O)N2C(=O)c3ccnc(N4CCN([C@@H]5CCN(Cc6ccc(Oc7cc8c(cc7F)c(N7CCC(=O)NC7=O)nn8C)cc6)CC5(F)F)CC4)c3C2=O)ccc1OC]

(S)-4-(4-(1-(2-(4-((3-(2,4-Dioxotetrahydropyrimidin-1(2H)-yl)-5-fluoro-1-methyl-1H-indazol-6-yl)oxy)phenyl)-2,2-difluoroethyl)piperidin-4-yl)piperazin-1-yl)-2-(1-(3-ethoxy-4-methoxyphenyl)-2-(methylsulfonyl)ethyl)-1H-pyrrolo[3,4-c]pyridine-1,3(2H)-dione B213.

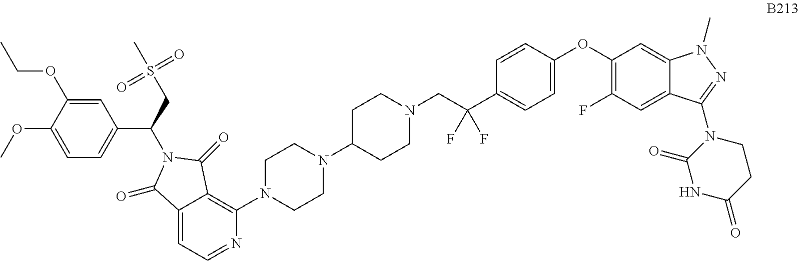 [SMILES: CCOc1cc([C@@H](CS(C)(=O)=O)N2C(=O)c3ccnc(N4CCN(C5CCN(CC(F)(F)c6ccc(Oc7cc8c(cc7F)c(N7CCC(=O)NC7=O)nn8C)cc6)CC5)CC4)c3C2=O)ccc1OC]

(S)-4-(4-(2-(4-(4-((3-(2,4-Dioxotetrahydropyrimidin-1(2H)-yl)-1-methyl-1H-indazol-6-yl)oxy)phenyl)piperazin-1-yl)ethyl)piperidin-1-yl)-2-(1-(3-ethoxy-4-methoxyphenyl)-2-(methylsulfonyl)ethyl)-1H-pyrrolo[3,4-c]pyridine-1,3(2H)-dione B214.

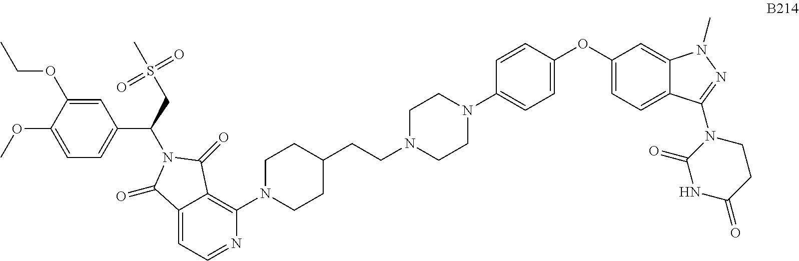 [SMILES: CCOc1cc([C@@H](CS(C)(=O)=O)N2C(=O)c3ccnc(N4CCC(CCN5CCN(c6ccc(Oc7ccc8c(N9CCC(=O)NC9=O)nn(C)c8c7)cc6)CC5)CC4)c3C2=O)ccc1OC]

(S)-4-(4-(2-(4-(4-((3-(2,4-Dioxotetrahydropyrimidin-1(2H)-yl)-5-fluoro-1-methyl-1H-indazol-6-yl)oxy)phenyl)piperazin-1-yl)ethyl)piperidin-1-yl)-2-(1-(3-ethoxy-4-methoxyphenyl)-2-(methylsulfonyl)ethyl)-1H-pyrrolo[3,4-c]pyridine-1,3(2H)-dione B215.

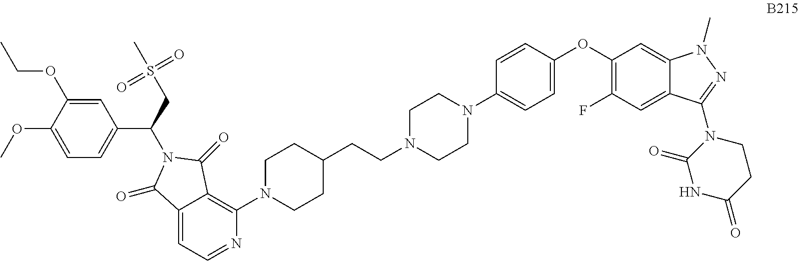 [SMILES: CCOc1cc([C@@H](CS(C)(=O)=O)N2C(=O)c3ccnc(N4CCC(CCN5CCN(c6ccc(Oc7cc8c(cc7F)c(N7CCC(=O)NC7=O)nn8C)cc6)CC5)CC4)c3C2=O)ccc1OC]

(S)-4-(4-(2-(1-(4-((3-(2,4-Dioxotetrahydropyrimidin-1(2H)-yl)-1-methyl-1H-indazol-6-yl)oxy)phenyl)piperidin-4-yl)ethyl)piperazin-1-yl)-2-(1-(3-ethoxy-4-methoxyphenyl)-2-(methylsulfonyl)ethyl)-1H-pyrrolo[3,4-c]pyridine-1,3(2H)-dione B216.

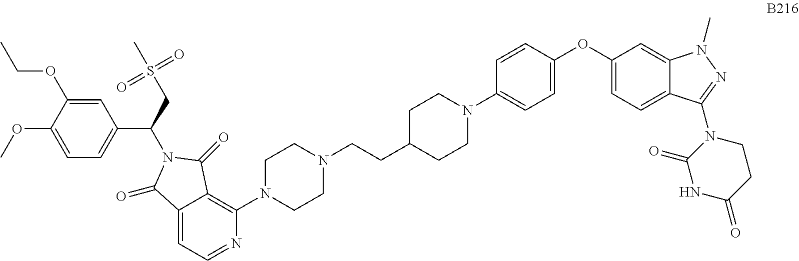 [SMILES: CCOc1cc([C@@H](CS(C)(=O)=O)N2C(=O)c3ccnc(N4CCN(CCC5CCN(c6ccc(Oc7ccc8c(N9CCC(=O)NC9=O)nn(C)c8c7)cc6)CC5)CC4)c3C2=O)ccc1OC]

(S)-4-(4-(2-(1-(4-((3-(2,4-Dioxotetrahydropyrimidin-1(2H)-yl)-5-fluoro-1-methyl-1H-indazol-6-yl)oxy)phenyl)piperidin-4-yl)ethyl)piperazin-1-yl)-2-(1-(3-ethoxy-4-methoxyphenyl)-2-(methylsulfonyl)ethyl)-1H-pyrrolo[3,4-c]pyridine-1,3(2H)-dione B217.

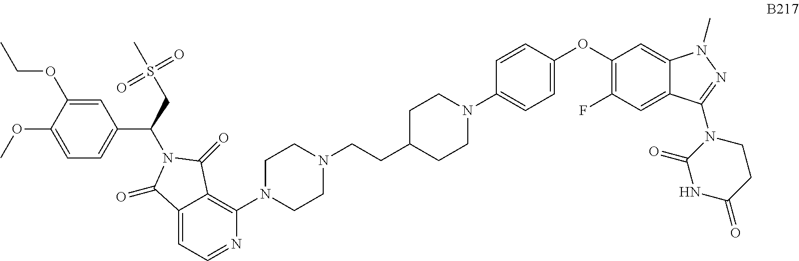 [SMILES: CCOc1cc([C@@H](CS(C)(=O)=O)N2C(=O)c3ccnc(N4CCN(CCC5CCN(c6ccc(Oc7cc8c(cc7F)c(N7CCC(=O)NC7=O)nn8C)cc6)CC5)CC4)c3C2=O)ccc1OC]

4-((S)-1′-(4-((3-(2,4-Dioxotetrahydropyrimidin-1(2H)-yl)-1-methyl-1H-indazol-6-yl)oxy)phenethyl)-3′,3′-difluoro-[4,4′-bipiperidin]-1-yl)-2-((S)-1-(3-ethoxy-4-methoxyphenyl)-2-(methylsulfonyl)ethyl)-1H-pyrrolo[3,4-c]pyridine-1,3(2H)-dione B218.

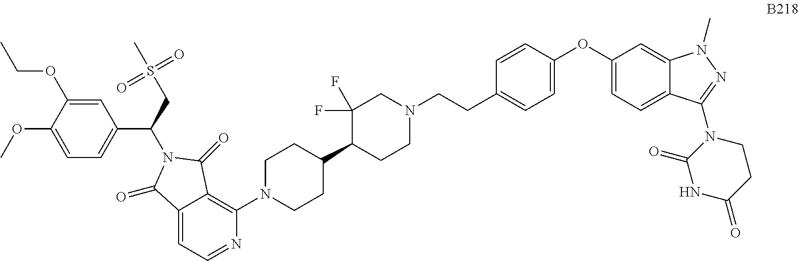 [SMILES: CCOc1cc([C@@H](CS(C)(=O)=O)N2C(=O)c3ccnc(N4CCC([C@@H]5CCN(CCc6ccc(Oc7ccc8c(N9CCC(=O)NC9=O)nn(C)c8c7)cc6)CC5(F)F)CC4)c3C2=O)ccc1OC]

4-((S)-1′-(4-((3-(2,4-Dioxotetrahydropyrimidin-1(2H)-yl)-5-fluoro-1-methyl-1H-indazol-6-yl)oxy)phenethyl)-3′,3′-difluoro-[4,4′-bipiperidin]-1-yl)-2-((S)-1-(3-ethoxy-4-methoxy-phenyl)-2-(methylsulfonyl)ethyl)-1H-pyrrolo[3,4-c]pyridine-1,3(2H)-dione B219.

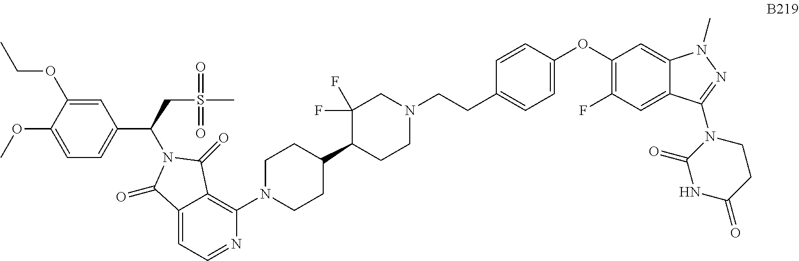 [SMILES: CCOc1cc([C@@H](CS(C)(=O)=O)N2C(=O)c3ccnc(N4CCC([C@@H]5CCN(CCc6ccc(Oc7cc8c(cc7F)c(N7CCC(=O)NC7=O)nn8C)cc6)CC5(F)F)CC4)c3C2=O)ccc1OC]

4-(4-((R)-1-(4-((3-(2,4-Dioxotetrahydropyrimidin-1(2H)-yl)-1-methyl-1H-indazol-6-yl)oxy)phenethyl)-3,3-difluoropiperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B220.

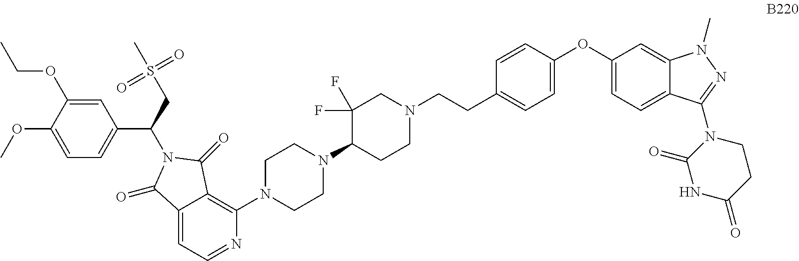 [SMILES: CCOc1cc([C@@H](CS(C)(=O)=O)N2C(=O)c3ccnc(N4CCN([C@@H]5CCN(CCc6ccc(Oc7ccc8c(N9CCC(=O)NC9=O)nn(C)c8c7)cc6)CC5(F)F)CC4)c3C2=O)ccc1OC]

4-(4-((R)-1-(4-((3-(2,4-Dioxotetrahydropyrimidin-1(2H)-yl)-5-fluoro-1-methyl-1H-indazol-6-yl)oxy)phenethyl)-3,3-difluoropiperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B221.

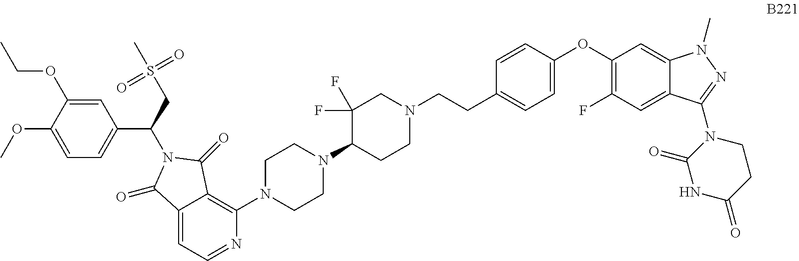 [SMILES: CCOc1cc([C@@H](CS(C)(=O)=O)N2C(=O)c3ccnc(N4CCN([C@@H]5CCN(CCc6ccc(Oc7cc8c(cc7F)c(N7CCC(=O)NC7=O)nn8C)cc6)CC5(F)F)CC4)c3C2=O)ccc1OC]

4-(4-((S)-1-(4-((3-(2,4-Dioxotetrahydropyrimidin-1(2H)-yl)-1-methyl-1H-indazol-6-yl)oxy)phenethyl)-3,3-difluoropiperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B222.

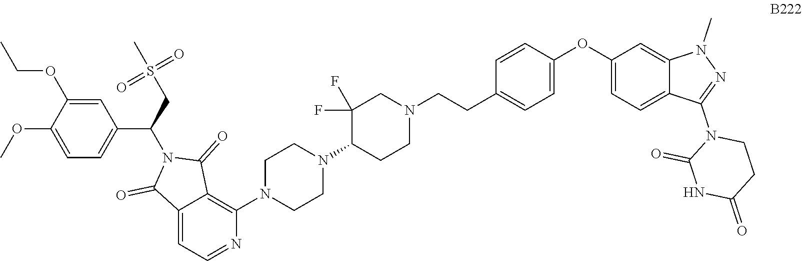 [SMILES: CCOc1cc([C@@H](CS(C)(=O)=O)N2C(=O)c3ccnc(N4CCN([C@H]5CCN(CCc6ccc(Oc7ccc8c(N9CCC(=O)NC9=O)nn(C)c8c7)cc6)CC5(F)F)CC4)c3C2=O)ccc1OC]

4-(4-((S)-1-(4-((3-(2,4-Dioxotetrahydropyrimidin-1(2H)-yl)-5-fluoro-1-methyl-1H-indazol-6-yl)oxy)phenethyl)-3,3-difluoropiperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B223.

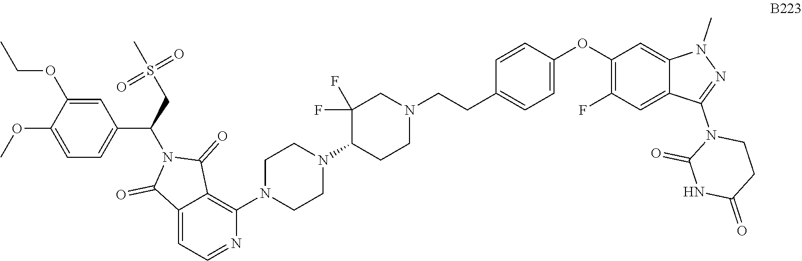 [SMILES: CCOc1cc([C@@H](CS(C)(=O)=O)N2C(=O)c3ccnc(N4CCN([C@H]5CCN(CCc6ccc(Oc7cc8c(cc7F)c(N7CCC(=O)NC7=O)nn8C)cc6)CC5(F)F)CC4)c3C2=O)ccc1OC]

(3R)-3-(5-(4-((4-((4-(4-(2,6-Dioxopiperidin-3-yl)phenyl)piperidin-1-yl)methyl)-piperidin-1-yl)methyl)piperidin-1-yl)-1,3-dioxoisoindolin-2-yl)-3-(3-ethoxy-4-methoxyphenyl)-propanenitrile C001.

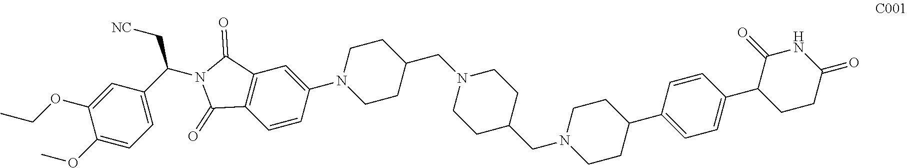 [SMILES: CCOc1cc([C@@H](CC#N)N2C(=O)c3ccc(N4CCC(CN5CCC(CN6CCC(c7ccc(C8CCC(=O)NC8=O)cc7)CC6)CC5)CC4)cc3C2=O)ccc1OC]

(3R)-3-(5-(4-((4-((4-(4-((2,6-Dioxopiperidin-3-yl)amino)phenyl)piperidin-1-yl)-methyl)piperidin-1-yl)methyl)piperidin-1-yl)-1,3-dioxoisoindolin-2-yl)-3-(3-ethoxy-4-methoxy-phenyl)propanenitrile C002.

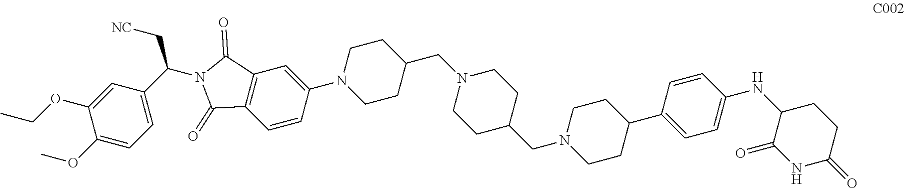 [SMILES: CCOc1cc([C@@H](CC#N)N2C(=O)c3ccc(N4CCC(CN5CCC(CN6CCC(c7ccc(NC8CCC(=O)NC8=O)cc7)CC6)CC5)CC4)cc3C2=O)ccc1OC]

(3R)-3-(5-(1′-(4-((3-(2,6-Dioxopiperidin-3-yl)-1-methyl-1H-indazol-6-yl)oxy)-benzyl)-[4,4′-bipiperidin]-1-yl)-1,3-dioxoisoindolin-2-yl)-3-(3-ethoxy-4-methoxyphenyl)-propanenitrile C012.

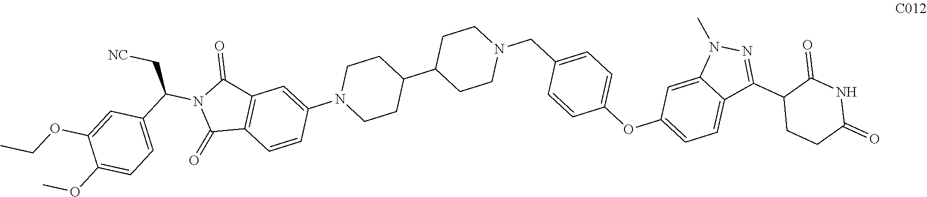 [SMILES: CCOc1cc([C@@H](CC#N)N2C(=O)c3ccc(N4CCC(C5CCN(Cc6ccc(Oc7ccc8c(C9CCC(=O)NC9=O)nn(C)c8c7)cc6)CC5)CC4)cc3C2=O)ccc1OC]

(3R)-3-(6-(1′-(4-((3-(2,6-Dioxopiperidin-3-yl)-1-methyl-1H-indazol-6-yl)oxy)-benzyl)-[4,4′-bipiperidin]-1-yl)-1-oxoisoindolin-2-yl)-3-(3-ethoxy-4-methoxyphenyl)propanenitrile C013.

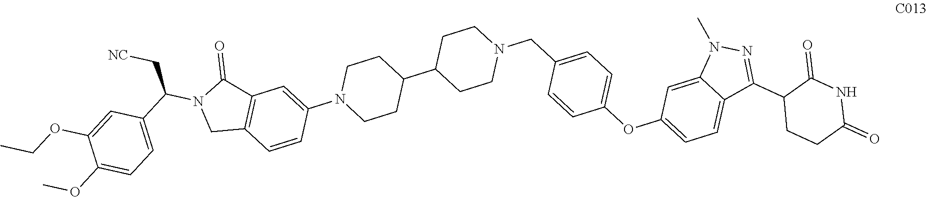 [SMILES: CCOc1cc([C@@H](CC#N)N2Cc3ccc(N4CCC(C5CCN(Cc6ccc(Oc7ccc8c(C9CCC(=O)NC9=O)nn(C)c8c7)cc6)CC5)CC4)cc3C2=O)ccc1OC]

(3R)-3-(5-(1′-(4-((3-(2,6-Dioxopiperidin-3-yl)-1-methyl-1H-indazol-6-yl)oxy)-benzyl)-[4,4′-bipiperidin]-1-yl)-1-oxoisoindolin-2-yl)-3-(3-ethoxy-4-methoxyphenyl)propanenitrile C014.

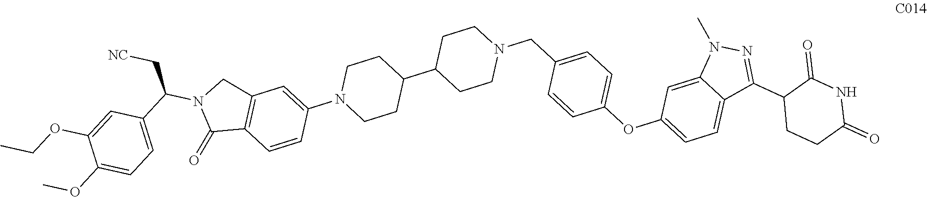 [SMILES: CCOc1cc([C@@H](CC#N)N2Cc3cc(N4CCC(C5CCN(Cc6ccc(Oc7ccc8c(C9CCC(=O)NC9=O)nn(C)c8c7)cc6)CC5)CC4)ccc3C2=O)ccc1OC]

(3R)-3-(4-(4-((1-(4-((3-(2,6-Dioxopiperidin-3-yl)-1-methyl-1H-indazol-6-yl)oxy)-benzyl)piperidin-4-yl)methyl)piperazin-1-yl)-1,3-dioxoisoindolin-2-yl)-3-(3-ethoxy-4-methoxy-phenyl)propanenitrile C015.

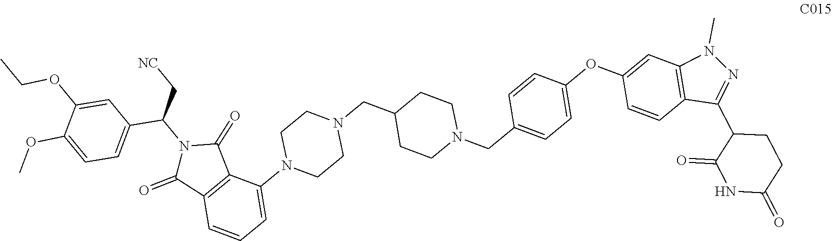 [SMILES: CCOc1cc([C@@H](CC#N)N2C(=O)c3cccc(N4CCN(CC5CCN(Cc6ccc(Oc7ccc8c(C9CCC(=O)NC9=O)nn(C)c8c7)cc6)CC5)CC4)c3C2=O)ccc1OC]

(3R)-3-(5-(4-((1-(4-((3-(2,6-Dioxopiperidin-3-yl)-1-methyl-1H-indazol-6-yl)oxy)-benzyl)piperidin-4-yl)methyl)piperazin-1-yl)-1,3-dioxoisoindolin-2-yl)-3-(3-ethoxy-4-methoxy-phenyl)propanenitrile C016.

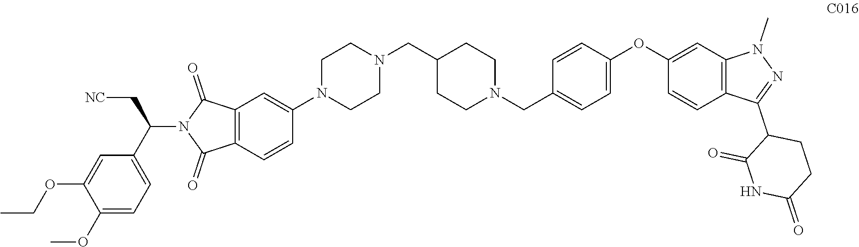 [SMILES: CCOc1cc([C@@H](CC#N)N2C(=O)c3ccc(N4CCN(CC5CCN(Cc6ccc(Oc7ccc8c(C9CCC(=O)NC9=O)nn(C)c8c7)cc6)CC5)CC4)cc3C2=O)ccc1OC]

(3R)-3-(5-(1-((1-(4-((3-(2,6-Dioxopiperidin-3-yl)-1-methyl-1H-indazol-6-yl)oxy)-benzyl)piperidin-4-yl)methyl)piperidin-4-yl)-1,3-dioxoisoindolin-2-yl)-3-(3-ethoxy-4-methoxy-phenyl)propanenitrile C017.

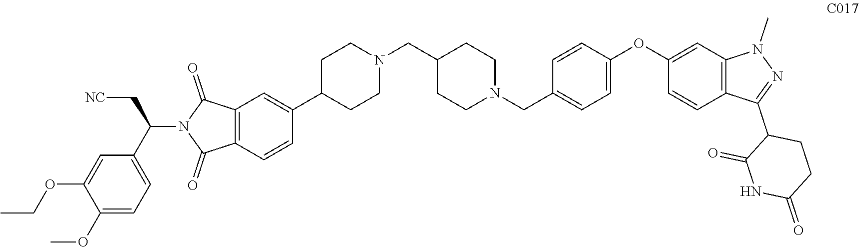 [SMILES: CCOc1cc([C@@H](CC#N)N2C(=O)c3ccc(C4CCN(CC5CCN(Cc6ccc(Oc7ccc8c(C9CCC(=O)NC9=O)nn(C)c8c7)cc6)CC5)CC4)cc3C2=O)ccc1OC]

(3R)-3-(4-(1′-(4-((3-(2,6-Dioxopiperidin-3-yl)-1-methyl-1H-indazol-6-yl)oxy)-benzyl)-[4,4′-bipiperidin]-1-yl)-1,3-dioxoisoindolin-2-yl)-3-(3-ethoxy-4-methoxyphenyl)-propanenitrile C018.

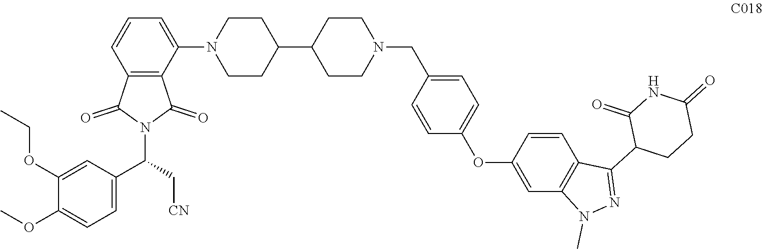 [SMILES: CCOc1cc([C@@H](CC#N)N2C(=O)c3cccc(N4CCC(C5CCN(Cc6ccc(Oc7ccc8c(C9CCC(=O)NC9=O)nn(C)c8c7)cc6)CC5)CC4)c3C2=O)ccc1OC]

(3R)-3-(5-(4-((4-((4-(3-(2,6-Dioxopiperidin-3-yl)-1-methyl-1H-indazol-6-yl)-piperidin-1-yl)methyl)piperidin-1-yl)methyl)piperidin-1-yl)-1,3-dioxoisoindolin-2-yl)-3-(3-ethoxy-4-methoxyphenyl)propanenitrile C019.

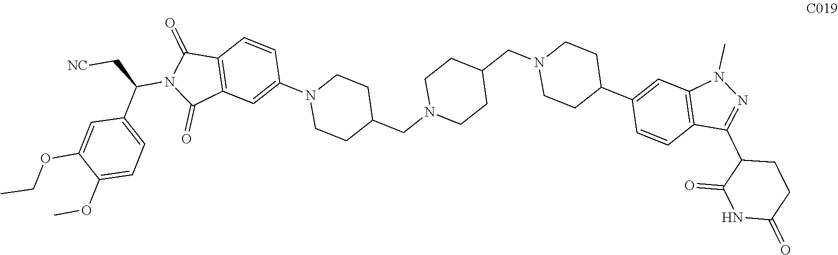 [SMILES: CCOc1cc([C@@H](CC#N)N2C(=O)c3ccc(N4CCC(CN5CCC(CN6CCC(c7ccc8c(C9CCC(=O)NC9=O)nn(C)c8c7)CC6)CC5)CC4)cc3C2=O)ccc1OC]

(3R)-3-(4-((1-((1-(2-(4-(3-(2,6-Dioxopiperidin-3-yl)-1-methyl-1H-indazol-6-yl)-piperazin-1-yl)ethyl)piperidin-4-yl)methyl)piperidin-4-yl)amino)-1,3-dioxoisoindolin-2-yl)-3-(3-ethoxy-4-methoxyphenyl)propanenitrile C020.

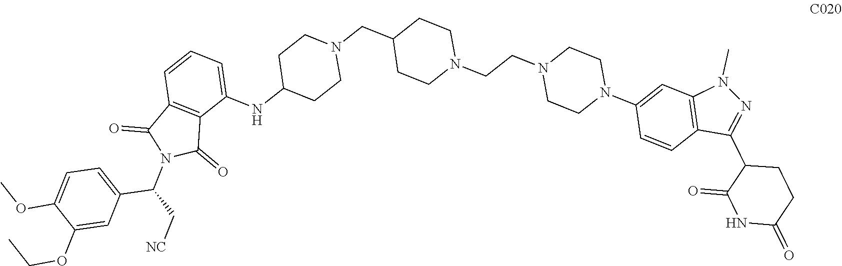 [SMILES: CCOc1cc([C@@H](CC#N)N2C(=O)c3cccc(NC4CCN(CC5CCN(CCN6CCN(c7ccc8c(C9CCC(=O)NC9=O)nn(C)c8c7)CC6)CC5)CC4)c3C2=O)ccc1OC]

(3R)-3-(4-(4-((1-(2-(4-(3-(2,6-Dioxopiperidin-3-yl)-1-methyl-1H-indazol-6-yl)-piperidin-1-yl)acetyl)piperidin-4-yl)methyl)piperazin-1-yl)-1,3-dioxoisoindolin-2-yl)-3-(3-ethoxy-4-methoxyphenyl)propanenitrile C021.

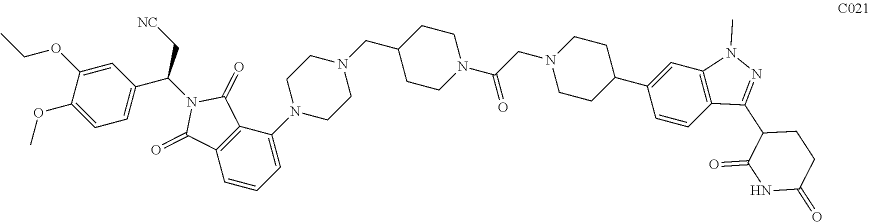 [SMILES: CCOc1cc([C@@H](CC#N)N2C(=O)c3cccc(N4CCN(CC5CCN(C(=O)CN6CCC(c7ccc8c(C9CCC(=O)NC9=O)nn(C)c8c7)CC6)CC5)CC4)c3C2=O)ccc1OC]

(3R)-3-(5-(4-((4-((3-(2,6-Dioxopiperidin-3-yl)-1-methyl-1H-indazol-6-yl)oxy)-piperidin-1-yl)methyl)-[1,4′-bipiperidin]-1′-yl)-1,3-dioxoisoindolin-2-yl)-3-(3-ethoxy-4-methoxyphenyl)propanenitrile C022.

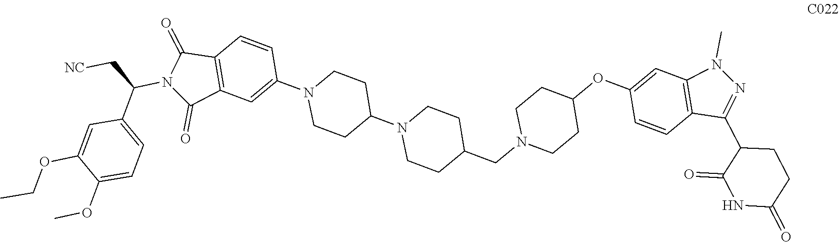 [SMILES: CCOc1cc([C@@H](CC#N)N2C(=O)c3ccc(N4CCC(N5CCC(CN6CCC(Oc7ccc8c(C9CCC(=O)NC9=O)nn(C)c8c7)CC6)CC5)CC4)cc3C2=O)ccc1OC]

2-(4-(11-(2-((R)-2-Cyano-1-(3-ethoxy-4-methoxyphenyl)ethyl)-1,3-dioxo-isoindolin-4-yl)undecyl)piperazin-1-yl)-N-(3-(2,6-dioxopiperidin-3-yl)-1-methyl-1H-indazol-6-yl)acetamide C023.

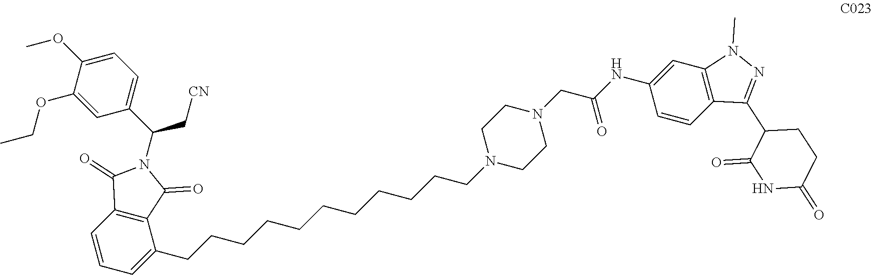 [SMILES: CCOc1cc([C@@H](CC#N)N2C(=O)c3cccc(CCCCCCCCCCCN4CCN(CC(=O)Nc5ccc6c(C7CCC(=O)NC7=O)nn(C)c6c5)CC4)c3C2=O)ccc1OC]

(R)-3-(6-(4-((S)-3,3-Difluoro-1-(4-((1-methyl-3-((R)-3-methyl-2,6-dioxo-piperidin-3-yl)-1H-indazol-6-yl)oxy)benzyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C031.

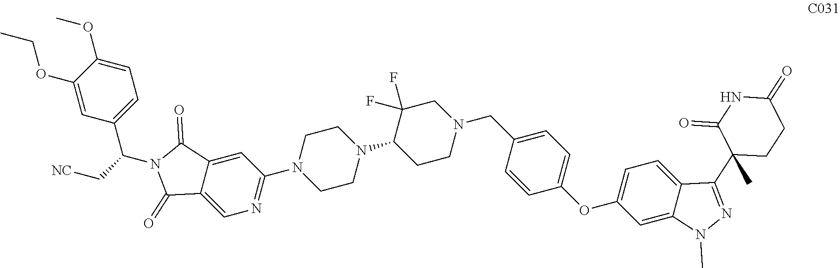 [SMILES: CCOc1cc([C@@H](CC#N)N2C(=O)c3cnc(N4CCN([C@H]5CCN(Cc6ccc(Oc7ccc8c([C@@]9(C)CCC(=O)NC9=O)nn(C)c8c7)cc6)CC5(F)F)CC4)cc3C2=O)ccc1OC]

(R)-3-(6-(4-((S)-3,3-Difluoro-1-(4-((5-fluoro-1-methyl-3-((R)-3-methyl-2,6-dioxopiperidin-3-yl)-1H-indazol-6-yl)oxy)benzyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C032.

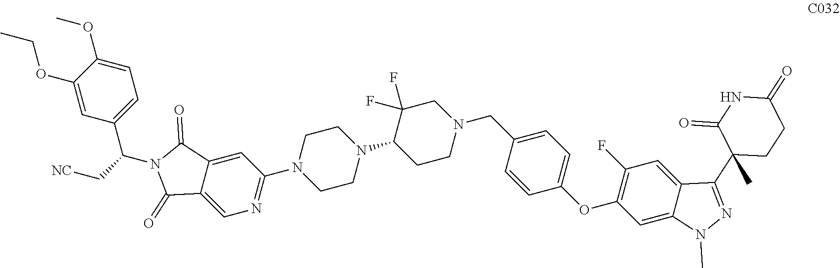 [SMILES: CCOc1cc([C@@H](CC#N)N2C(=O)c3cnc(N4CCN([C@H]5CCN(Cc6ccc(Oc7cc8c(cc7F)c([C@@]7(C)CCC(=O)NC7=O)nn8C)cc6)CC5(F)F)CC4)cc3C2=O)ccc1OC]

(R)-3-(6-(4-((R)-3,3-Difluoro-1-(4-((1-methyl-3-((R)-3-methyl-2,6-dioxo-piperidin-3-yl)-1H-indazol-6-yl)oxy)benzyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C033.

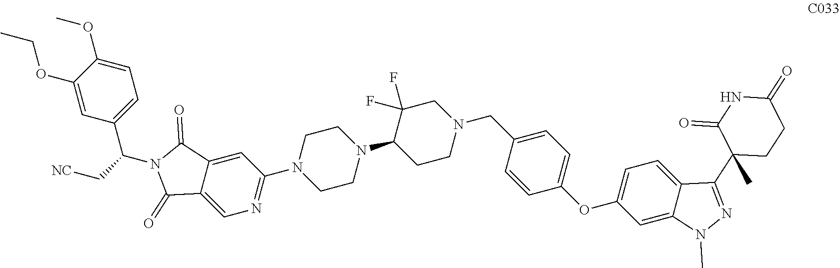 [SMILES: CCOc1cc([C@@H](CC#N)N2C(=O)c3cnc(N4CCN([C@@H]5CCN(Cc6ccc(Oc7ccc8c([C@@]9(C)CCC(=O)NC9=O)nn(C)c8c7)cc6)CC5(F)F)CC4)cc3C2=O)ccc1OC]

(R)-3-(6-(4-((R)-3,3-Difluoro-1-(4-((5-fluoro-1-methyl-3-((R)-3-methyl-2,6-dioxopiperidin-3-yl)-1H-indazol-6-yl)oxy)benzyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C034.

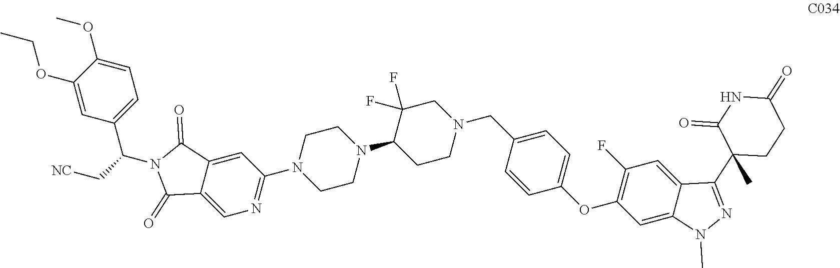 [SMILES: CCOc1cc([C@@H](CC#N)N2C(=O)c3cnc(N4CCN([C@@H]5CCN(Cc6ccc(Oc7cc8c(cc7F)c([C@@]7(C)CCC(=O)NC7=O)nn8C)cc6)CC5(F)F)CC4)cc3C2=O)ccc1OC]

(R)-3-(4-(4-((S)-3,3-Difluoro-1-(4-((1-methyl-3-((R)-3-methyl-2,6-dioxo-piperidin-3-yl)-1H-indazol-6-yl)oxy)benzyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C035.

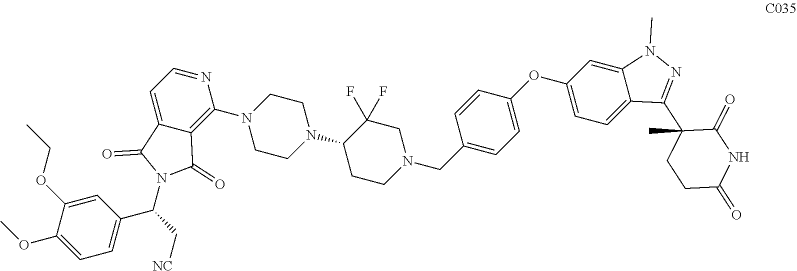 [SMILES: CCOc1cc([C@@H](CC#N)N2C(=O)c3ccnc(N4CCN([C@H]5CCN(Cc6ccc(Oc7ccc8c([C@@]9(C)CCC(=O)NC9=O)nn(C)c8c7)cc6)CC5(F)F)CC4)c3C2=O)ccc1OC]

(R)-3-(4-(4-((S)-3,3-Difluoro-1-(4-((5-fluoro-1-methyl-3-((R)-3-methyl-2,6-dioxopiperidin-3-yl)-1H-indazol-6-yl)oxy)benzyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C036.

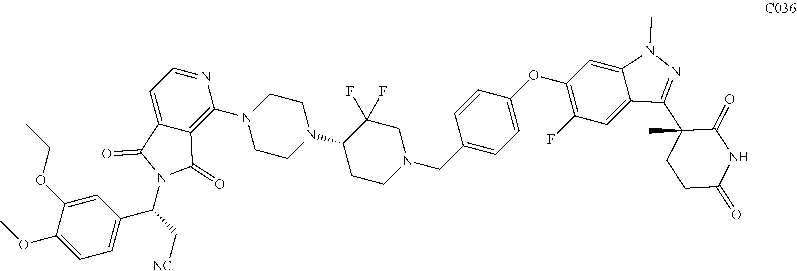 [SMILES: CCOc1cc([C@@H](CC#N)N2C(=O)c3ccnc(N4CCN([C@H]5CCN(Cc6ccc(Oc7cc8c(cc7F)c([C@@]7(C)CCC(=O)NC7=O)nn8C)cc6)CC5(F)F)CC4)c3C2=O)ccc1OC]

(R)-3-(4-(4-((R)-3,3-Difluoro-1-(4-((1-methyl-3-((R)-3-methyl-2,6-dioxo-piperidin-3-yl)-1H-indazol-6-yl)oxy)benzyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C037.

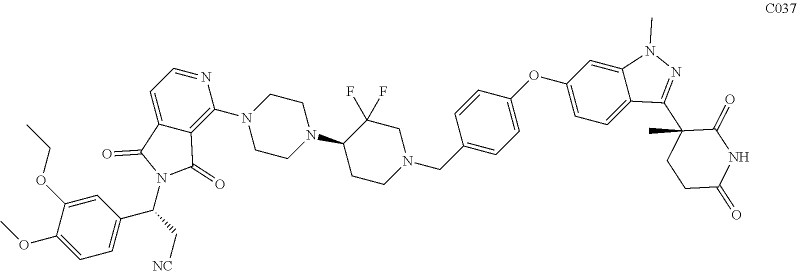 [SMILES: CCOc1cc([C@@H](CC#N)N2C(=O)c3ccnc(N4CCN([C@@H]5CCN(Cc6ccc(Oc7ccc8c([C@@]9(C)CCC(=O)NC9=O)nn(C)c8c7)cc6)CC5(F)F)CC4)c3C2=O)ccc1OC]

(R)-3-(4-(4-((R)-3,3-Difluoro-1-(4-((5-fluoro-1-methyl-3-((R)-3-methyl-2,6-dioxopiperidin-3-yl)-1H-indazol-6-yl)oxy)benzyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C038.

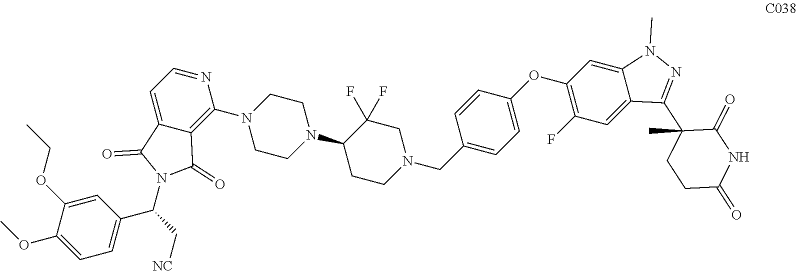 [SMILES: CCOc1cc([C@@H](CC#N)N2C(=O)c3ccnc(N4CCN([C@@H]5CCN(Cc6ccc(Oc7cc8c(cc7F)c([C@@]7(C)CCC(=O)NC7=O)nn8C)cc6)CC5(F)F)CC4)c3C2=O)ccc1OC]

(R)-3-(4-(4-(1-(2,2-Difluoro-2-(4-((1-methyl-3-((R)-3-methyl-2,6-dioxopiperidin-3-yl)-1H-indazol-6-yl)oxy)phenyl)ethyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C039.

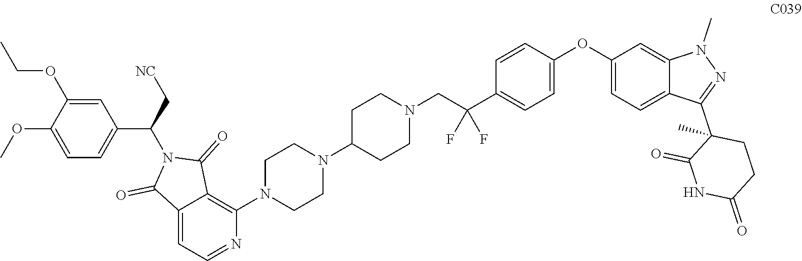 [SMILES: CCOc1cc([C@@H](CC#N)N2C(=O)c3ccnc(N4CCN(C5CCN(CC(F)(F)c6ccc(Oc7ccc8c([C@@]9(C)CCC(=O)NC9=O)nn(C)c8c7)cc6)CC5)CC4)c3C2=O)ccc1OC]

(R)-3-(4-(4-(1-(2,2-Difluoro-2-(4-((5-fluoro-1-methyl-3-((R)-3-methyl-2,6-dioxo-piperidin-3-yl)-1H-indazol-6-yl)oxy)phenyl)ethyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C040.

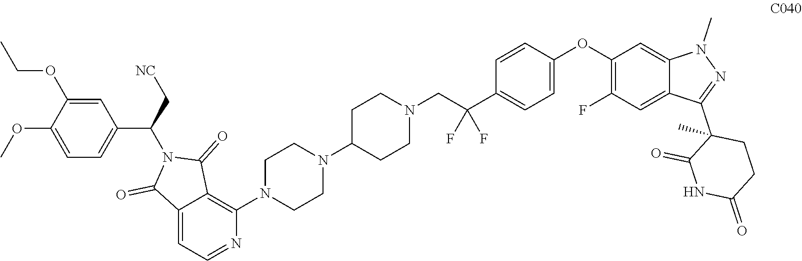 [SMILES: CCOc1cc([C@@H](CC#N)N2C(=O)c3ccnc(N4CCN(C5CCN(CC(F)(F)c6ccc(Oc7cc8c(cc7F)c([C@@]7(C)CCC(=O)NC7=O)nn8C)cc6)CC5)CC4)c3C2=O)ccc1OC]

(R)-3-(3-Ethoxy-4-methoxyphenyl)-3-(4-(4-(2-(1-(4-((1-methyl-3-((R)-3-methyl-2,6-dioxopiperidin-3-yl)-1H-indazol-6-yl)oxy)phenyl)piperidin-4-yl)ethyl)piperazin-1-yl)-1,3-dioxo-1,3-dihydro-2H-pyrrolo[3,4-c]pyridin-2-yl)propanenitrile C041.

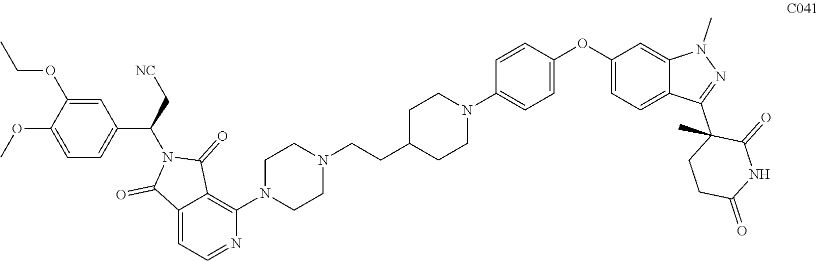 [SMILES: CCOc1cc([C@@H](CC#N)N2C(=O)c3ccnc(N4CCN(CCC5CCN(c6ccc(Oc7ccc8c([C@@]9(C)CCC(=O)NC9=O)nn(C)c8c7)cc6)CC5)CC4)c3C2=O)ccc1OC]

(R)-3-(3-Ethoxy-4-methoxyphenyl)-3-(4-(4-(2-(1-(4-((5-fluoro-1-methyl-3-((R)-3-methyl-2,6-dioxopiperidin-3-yl)-1H-indazol-6-yl)oxy)phenyl)piperidin-4-yl)ethyl)piperazin-1-yl)-1,3-dioxo-1,3-dihydro-2H-pyrrolo[3,4-c]pyridin-2-yl)propanenitrile C042.

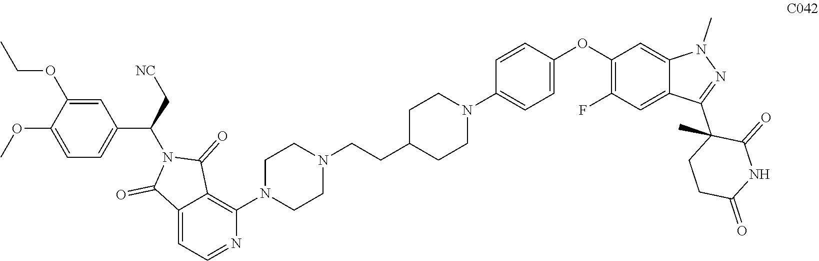 [SMILES: CCOc1cc([C@@H](CC#N)N2C(=O)c3ccnc(N4CCN(CCC5CCN(c6ccc(Oc7cc8c(cc7F)c([C@@]7(C)CCC(=O)NC7=O)nn8C)cc6)CC5)CC4)c3C2=O)ccc1OC]

(R)-3-(5-(4-((S)-1-(4-((3-(2,4-Dioxotetrahydropyrimidin-1(2H)-yl)-1-methyl-1H-indazol-6-yl)oxy)benzyl)-3,3-difluoropiperidin-4-yl)piperazin-1-yl)-1,3-dioxoisoindolin-2-yl)-3-(3-ethoxy-4-methoxyphenyl)propanenitrile C051.

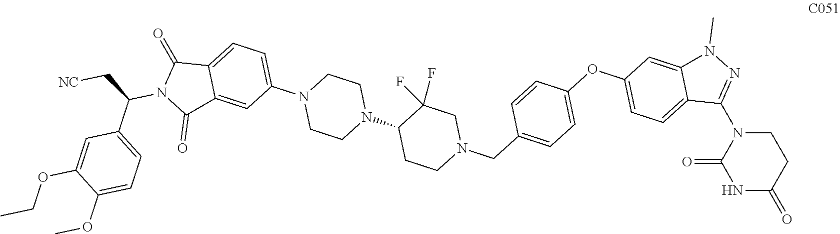 [SMILES: CCOc1cc([C@@H](CC#N)N2C(=O)c3ccc(N4CCN([C@H]5CCN(Cc6ccc(Oc7ccc8c(N9CCC(=O)NC9=O)nn(C)c8c7)cc6)CC5(F)F)CC4)cc3C2=O)ccc1OC]

(R)-3-(5-(4-((R)-1-(4-((3-(2,4-Dioxotetrahydropyrimidin-1(2H)-yl)-1-methyl-1H-indazol-6-yl)oxy)benzyl)-3,3-difluoropiperidin-4-yl)piperazin-1-yl)-1,3-dioxoisoindolin-2-yl)-3-(3-ethoxy-4-methoxyphenyl)propanenitrile C052.

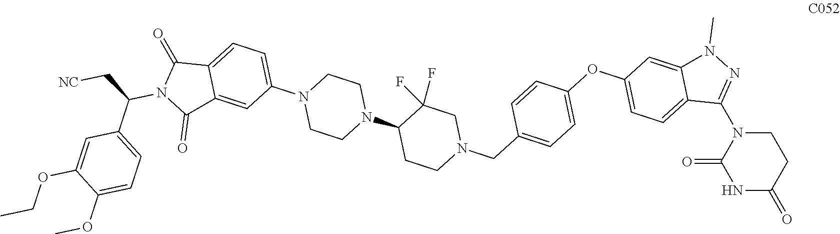 [SMILES: CCOc1cc([C@@H](CC#N)N2C(=O)c3ccc(N4CCN([C@@H]5CCN(Cc6ccc(Oc7ccc8c(N9CCC(=O)NC9=O)nn(C)c8c7)cc6)CC5(F)F)CC4)cc3C2=O)ccc1OC]

(R)-3-(5-(4-((S)-1-(4-((3-(2,4-Dioxotetrahydropyrimidin-1(2H)-yl)-5-fluoro-1-methyl-1H-indazol-6-yl)oxy)benzyl)-3,3-difluoropiperidin-4-yl)piperazin-1-yl)-1,3-dioxo-isoindolin-2-yl)-3-(3-ethoxy-4-methoxyphenyl)propanenitrile C053.

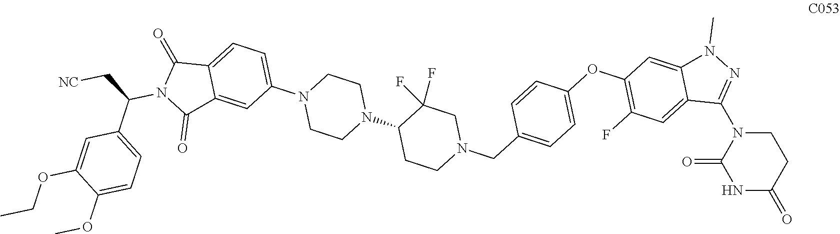 [SMILES: CCOc1cc([C@@H](CC#N)N2C(=O)c3ccc(N4CCN([C@H]5CCN(Cc6ccc(Oc7cc8c(cc7F)c(N7CCC(=O)NC7=O)nn8C)cc6)CC5(F)F)CC4)cc3C2=O)ccc1OC]

(R)-3-(5-(4-((R)-1-(4-((3-(2,4-Dioxotetrahydropyrimidin-1(2H)-yl)-5-fluoro-1-methyl-1H-indazol-6-yl)oxy)benzyl)-3,3-difluoropiperidin-4-yl)piperazin-1-yl)-1,3-dioxo-isoindolin-2-yl)-3-(3-ethoxy-4-methoxyphenyl)propanenitrile C054.

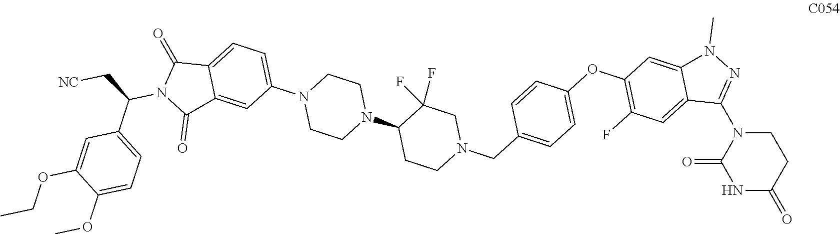 [SMILES: CCOc1cc([C@@H](CC#N)N2C(=O)c3ccc(N4CCN([C@@H]5CCN(Cc6ccc(Oc7cc8c(cc7F)c(N7CCC(=O)NC7=O)nn8C)cc6)CC5(F)F)CC4)cc3C2=O)ccc1OC]

(R)-3-(4-(4-((S)-1-(4-((3-(2,4-Dioxotetrahydropyrimidin-1(2H)-yl)-1-methyl-1H-indazol-6-yl)oxy)benzyl)-3,3-difluoropiperidin-4-yl)piperazin-1-yl)-1,3-dioxoisoindolin-2-yl)-3-(3-ethoxy-4-methoxyphenyl)propanenitrile C055.

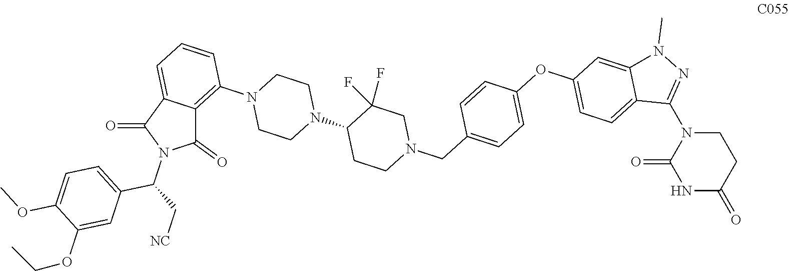 [SMILES: CCOc1cc([C@@H](CC#N)N2C(=O)c3cccc(N4CCN([C@H]5CCN(Cc6ccc(Oc7ccc8c(N9CCC(=O)NC9=O)nn(C)c8c7)cc6)CC5(F)F)CC4)c3C2=O)ccc1OC]

(R)-3-(4-(4-((S)-1-(4-((3-(2,4-Dioxotetrahydropyrimidin-1(2H)-yl)-5-fluoro-1-methyl-1H-indazol-6-yl)oxy)benzyl)-3,3-difluoropiperidin-4-yl)piperazin-1-yl)-1,3-dioxo-isoindolin-2-yl)-3-(3-ethoxy-4-methoxyphenyl)propanenitrile C056.

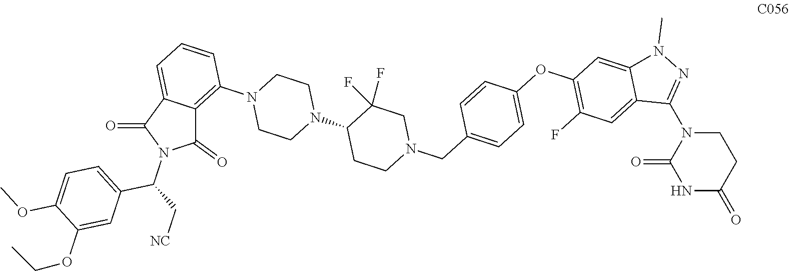 [SMILES: CCOc1cc([C@@H](CC#N)N2C(=O)c3cccc(N4CCN([C@H]5CCN(Cc6ccc(Oc7cc8c(cc7F)c(N7CCC(=O)NC7=O)nn8C)cc6)CC5(F)F)CC4)c3C2=O)ccc1OC]

(R)-3-(4-(4-((R)-1-(4-((3-(2,4-Dioxotetrahydropyrimidin-1(2H)-yl)-1-methyl-1H-indazol-6-yl)oxy)benzyl)-3,3-difluoropiperidin-4-yl)piperazin-1-yl)-1,3-dioxoisoindolin-2-yl)-3-(3-ethoxy-4-methoxyphenyl)propanenitrile C057.

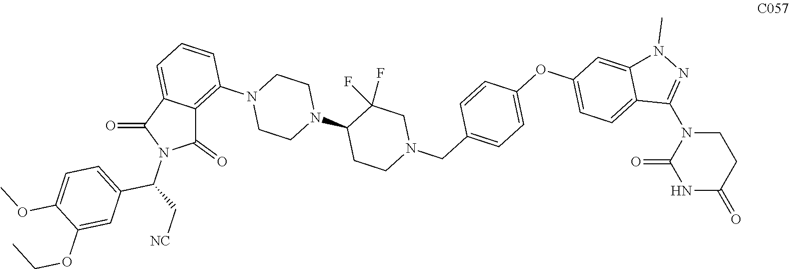 [SMILES: CCOc1cc([C@@H](CC#N)N2C(=O)c3cccc(N4CCN([C@@H]5CCN(Cc6ccc(Oc7ccc8c(N9CCC(=O)NC9=O)nn(C)c8c7)cc6)CC5(F)F)CC4)c3C2=O)ccc1OC]

(R)-3-(4-(4-((R)-1-(4-((3-(2,4-Dioxotetrahydropyrimidin-1(2H)-yl)-5-fluoro-1-methyl-1H-indazol-6-yl)oxy)benzyl)-3,3-difluoropiperidin-4-yl)piperazin-1-yl)-1,3-dioxo-isoindolin-2-yl)-3-(3-ethoxy-4-methoxyphenyl)propanenitrile C058.

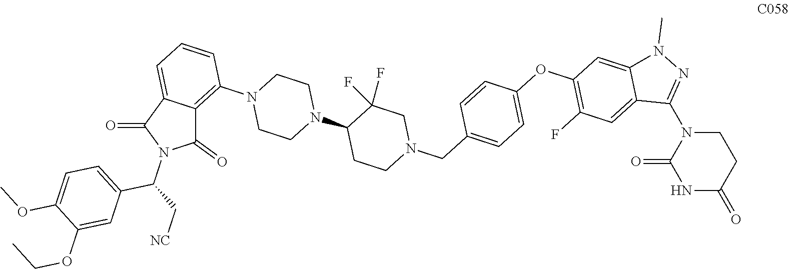 [SMILES: CCOc1cc([C@@H](CC#N)N2C(=O)c3cccc(N4CCN([C@@H]5CCN(Cc6ccc(Oc7cc8c(cc7F)c(N7CCC(=O)NC7=O)nn8C)cc6)CC5(F)F)CC4)c3C2=O)ccc1OC]

(R)-3-(4-(4-((4-(4-((3-(2,4-Dioxotetrahydropyrimidin-1(2H)-yl)-1-methyl-1H-indazol-6-yl)oxy)phenyl)piperidin-1-yl)methyl)-4-fluoropiperidin-1-yl)-1,3-dioxoisoindolin-2-yl)-3-(3-ethoxy-4-methoxyphenyl)propanenitrile C059.

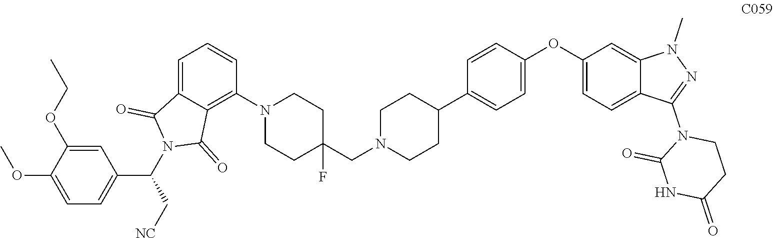 [SMILES: CCOc1cc([C@@H](CC#N)N2C(=O)c3cccc(N4CCC(F)(CN5CCC(c6ccc(Oc7ccc8c(N9CCC(=O)NC9=O)nn(C)c8c7)cc6)CC5)CC4)c3C2=O)ccc1OC]

(R)-3-(4-(4-((4-(4-((3-(2,4-Dioxotetrahydropyrimidin-1(2H)-yl)-5-fluoro-1-methyl-1H-indazol-6-yl)oxy)phenyl)piperidin-1-yl)methyl)-4-fluoropiperidin-1-yl)-1,3-dioxo-isoindolin-2-yl)-3-(3-ethoxy-4-methoxyphenyl)propanenitrile C060.

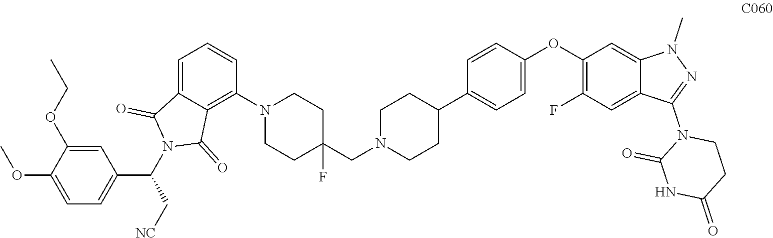 [SMILES: CCOc1cc([C@@H](CC#N)N2C(=O)c3cccc(N4CCC(F)(CN5CCC(c6ccc(Oc7cc8c(cc7F)c(N7CCC(=O)NC7=O)nn8C)cc6)CC5)CC4)c3C2=O)ccc1OC]

(R)-3-(5-(4-((4-(4-((3-(2,4-Dioxotetrahydropyrimidin-1(2H)-yl)-1-methyl-1H-indazol-6-yl)oxy)phenyl)piperidin-1-yl)methyl)-4-fluoropiperidin-1-yl)-1,3-dioxoisoindolin-2-yl)-3-(3-ethoxy-4-methoxyphenyl)propanenitrile C061.

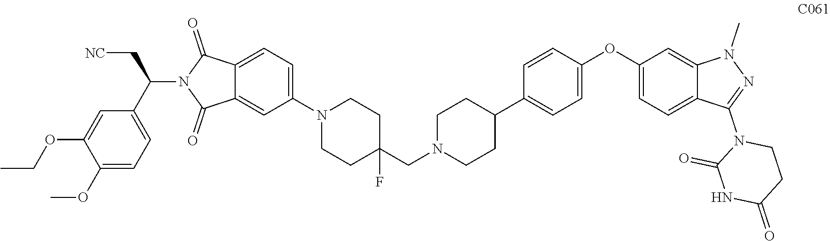 [SMILES: CCOc1cc([C@@H](CC#N)N2C(=O)c3ccc(N4CCC(F)(CN5CCC(c6ccc(Oc7ccc8c(N9CCC(=O)NC9=O)nn(C)c8c7)cc6)CC5)CC4)cc3C2=O)ccc1OC]

(R)-3-(5-(4-((4-(4-((3-(2,4-Dioxotetrahydropyrimidin-1(2H)-yl)-5-fluoro-1-methyl-1H-indazol-6-yl)oxy)phenyl)piperidin-1-yl)methyl)-4-fluoropiperidin-1-yl)-1,3-dioxoisoindolin-2-yl)-3-(3-ethoxy-4-methoxyphenyl)propanenitrile C062.

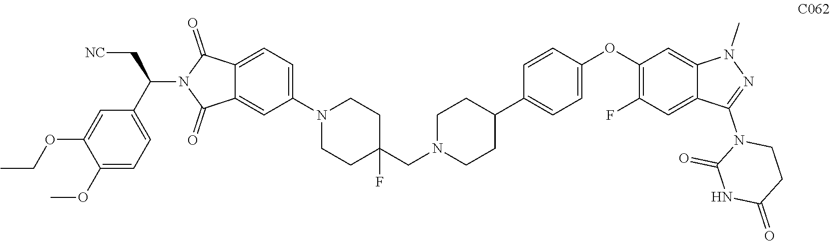 [SMILES: CCOc1cc([C@@H](CC#N)N2C(=O)c3ccc(N4CCC(F)(CN5CCC(c6ccc(Oc7cc8c(cc7F)c(N7CCC(=O)NC7=O)nn8C)cc6)CC5)CC4)cc3C2=O)ccc1OC]

(R)-3-(4-(4-(1-(2-(4-((3-(2,4-Dioxotetrahydropyrimidin-1(2H)-yl)-1-methyl-1H-indazol-6-yl)oxy)phenyl)-2,2-difluoroethyl)piperidin-4-yl)piperazin-1-yl)-1,3-dioxoisoindolin-2-yl)-3-(3-ethoxy-4-methoxyphenyl)propanenitrile C063.

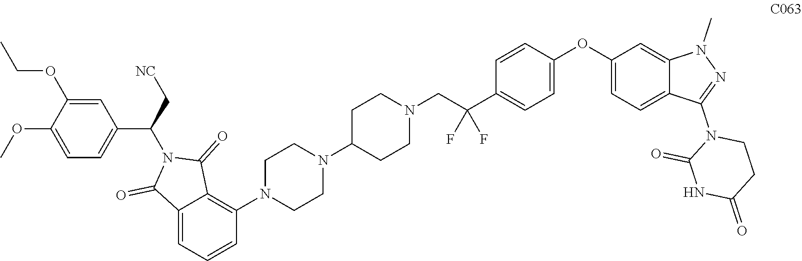 [SMILES: CCOc1cc([C@@H](CC#N)N2C(=O)c3cccc(N4CCN(C5CCN(CC(F)(F)c6ccc(Oc7ccc8c(N9CCC(=O)NC9=O)nn(C)c8c7)cc6)CC5)CC4)c3C2=O)ccc1OC]

(R)-3-(4-(4-(1-(2-(4-((3-(2,4-Dioxotetrahydropyrimidin-1(2H)-yl)-5-fluoro-1-methyl-1H-indazol-6-yl)oxy)phenyl)-2,2-difluoroethyl)piperidin-4-yl)piperazin-1-yl)-1,3-dioxo-isoindolin-2-yl)-3-(3-ethoxy-4-methoxyphenyl)propanenitrile C064.

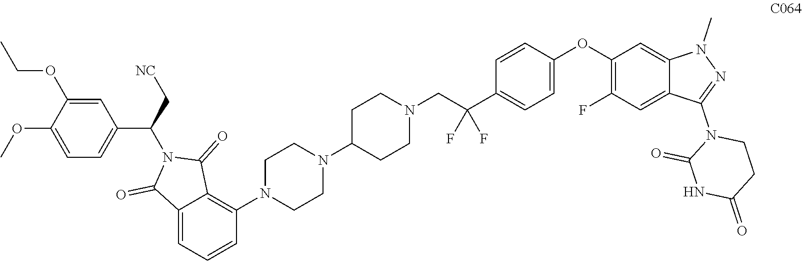 [SMILES: CCOc1cc([C@@H](CC#N)N2C(=O)c3cccc(N4CCN(C5CCN(CC(F)(F)c6ccc(Oc7cc8c(cc7F)c(N7CCC(=O)NC7=O)nn8C)cc6)CC5)CC4)c3C2=O)ccc1OC]

(R)-3-(4-(4-(2-(4-(4-((3-(2,4-Dioxotetrahydropyrimidin-1(2H)-yl)-1-methyl-1H-indazol-6-yl)oxy)phenyl)piperazin-1-yl)ethyl)piperidin-1-yl)-1,3-dioxoisoindolin-2-yl)-3-(3-ethoxy-4-methoxyphenyl)propanenitrile C065.

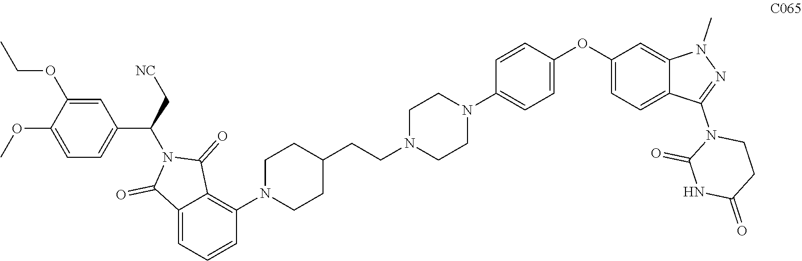 [SMILES: CCOc1cc([C@@H](CC#N)N2C(=O)c3cccc(N4CCC(CCN5CCN(c6ccc(Oc7ccc8c(N9CCC(=O)NC9=O)nn(C)c8c7)cc6)CC5)CC4)c3C2=O)ccc1OC]

(R)-3-(4-(4-(2-(4-(4-((3-(2,4-Dioxotetrahydropyrimidin-1(2H)-yl)-5-fluoro-1-methyl-1H-indazol-6-yl)oxy)phenyl)piperazin-1-yl)ethyl)piperidin-1-yl)-1,3-dioxoisoindolin-2-yl)-3-(3-ethoxy-4-methoxyphenyl)propanenitrile C066.

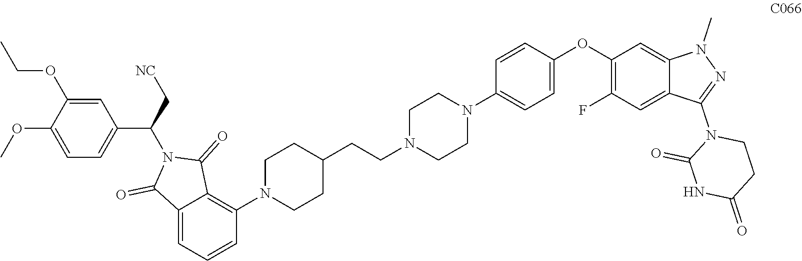 [SMILES: CCOc1cc([C@@H](CC#N)N2C(=O)c3cccc(N4CCC(CCN5CCN(c6ccc(Oc7cc8c(cc7F)c(N7CCC(=O)NC7=O)nn8C)cc6)CC5)CC4)c3C2=O)ccc1OC]

(R)-3-(4-(4-(2-(1-(4-((3-(2,4-Dioxotetrahydropyrimidin-1(2H)-yl)-1-methyl-1H-indazol-6-yl)oxy)phenyl)piperidin-4-yl)ethyl)piperazin-1-yl)-1,3-dioxoisoindolin-2-yl)-3-(3-ethoxy-4-methoxyphenyl)propanenitrile C067.

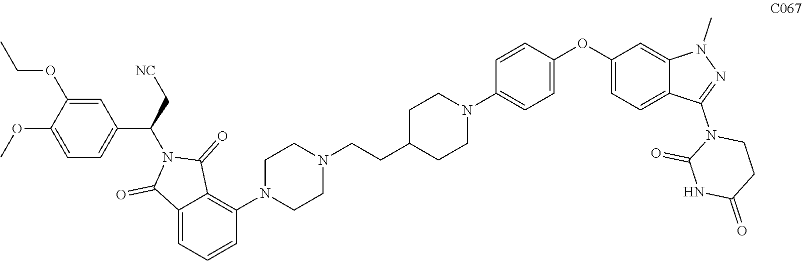 [SMILES: CCOc1cc([C@@H](CC#N)N2C(=O)c3cccc(N4CCN(CCC5CCN(c6ccc(Oc7ccc8c(N9CCC(=O)NC9=O)nn(C)c8c7)cc6)CC5)CC4)c3C2=O)ccc1OC]

(R)-3-(4-(4-(2-(1-(4-((3-(2,4-Dioxotetrahydropyrimidin-1(2H)-yl)-5-fluoro-1-methyl-1H-indazol-6-yl)oxy)phenyl)piperidin-4-yl)ethyl)piperazin-1-yl)-1,3-dioxoisoindolin-2-yl)-3-(3-ethoxy-4-methoxyphenyl)propanenitrile C068.

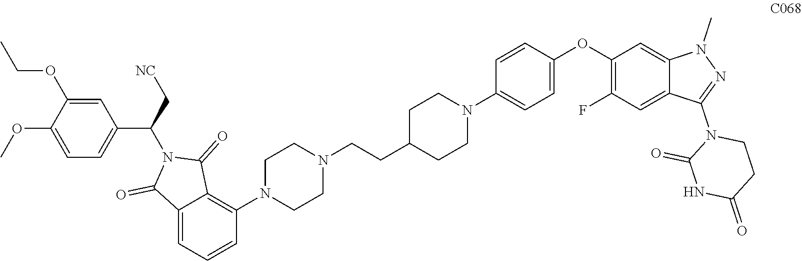 [SMILES: CCOc1cc([C@@H](CC#N)N2C(=O)c3cccc(N4CCN(CCC5CCN(c6ccc(Oc7cc8c(cc7F)c(N7CCC(=O)NC7=O)nn8C)cc6)CC5)CC4)c3C2=O)ccc1OC]

(R)-3-(4-(4-((R)-1-(4-((3-(2,4-Dioxotetrahydropyrimidin-1(2H)-yl)-1-methyl-1H-indazol-6-yl)oxy)phenethyl)-3,3-difluoropiperidin-4-yl)piperazin-1-yl)-1,3-dioxoisoindolin-2-yl)-3-(3-ethoxy-4-methoxyphenyl)propanenitrile C069.

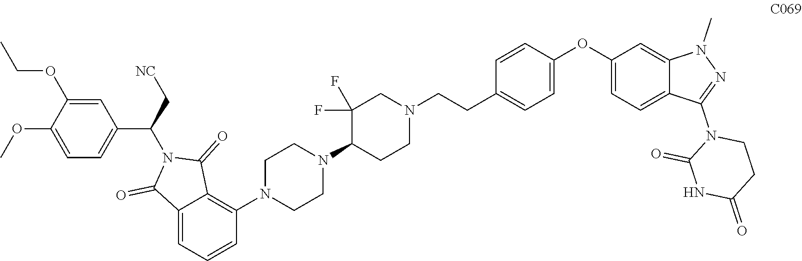 [SMILES: CCOc1cc([C@@H](CC#N)N2C(=O)c3cccc(N4CCN([C@@H]5CCN(CCc6ccc(Oc7ccc8c(N9CCC(=O)NC9=O)nn(C)c8c7)cc6)CC5(F)F)CC4)c3C2=O)ccc1OC]

(R)-3-(4-(4-((R)-1-(4-((3-(2,4-Dioxotetrahydropyrimidin-1(2H)-yl)-5-fluoro-1-methyl-1H-indazol-6-yl)oxy)phenethyl)-3,3-difluoropiperidin-4-yl)piperazin-1-yl)-1,3-dioxo-isoindolin-2-yl)-3-(3-ethoxy-4-methoxyphenyl)propanenitrile C070.

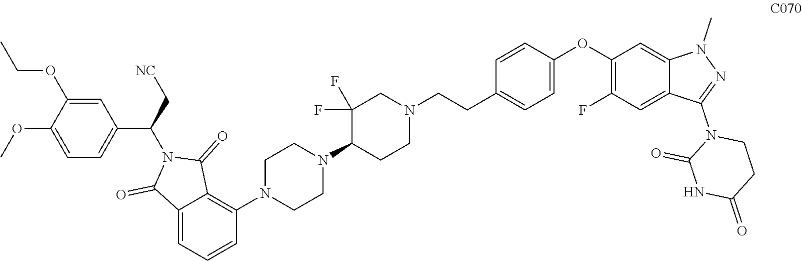 [SMILES: CCOc1cc([C@@H](CC#N)N2C(=O)c3cccc(N4CCN([C@@H]5CCN(CCc6ccc(Oc7cc8c(cc7F)c(N7CCC(=O)NC7=O)nn8C)cc6)CC5(F)F)CC4)c3C2=O)ccc1OC]

(R)-3-(4-(4-((S)-1-(4-((3-(2,4-Dioxotetrahydropyrimidin-1(2H)-yl)-1-methyl-1H-indazol-6-yl)oxy)phenethyl)-3,3-difluoropiperidin-4-yl)piperazin-1-yl)-1,3-dioxoisoindolin-2-yl)-3-(3-ethoxy-4-methoxyphenyl)propanenitrile C071.

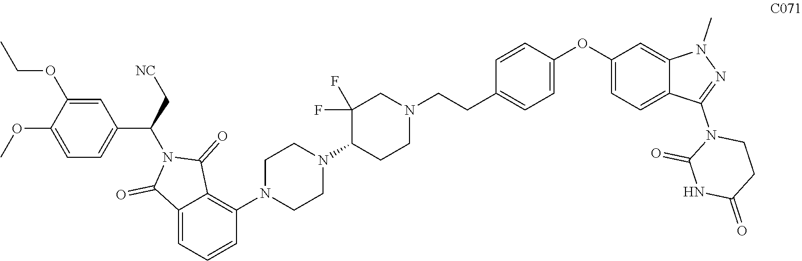 [SMILES: CCOc1cc([C@@H](CC#N)N2C(=O)c3cccc(N4CCN([C@H]5CCN(CCc6ccc(Oc7ccc8c(N9CCC(=O)NC9=O)nn(C)c8c7)cc6)CC5(F)F)CC4)c3C2=O)ccc1OC]

(R)-3-(4-(4-((S)-1-(4-((3-(2,4-Dioxotetrahydropyrimidin-1(2H)-yl)-5-fluoro-1-methyl-1H-indazol-6-yl)oxy)phenethyl)-3,3-difluoropiperidin-4-yl)piperazin-1-yl)-1,3-dioxo-isoindolin-2-yl)-3-(3-ethoxy-4-methoxyphenyl)propanenitrile C072.

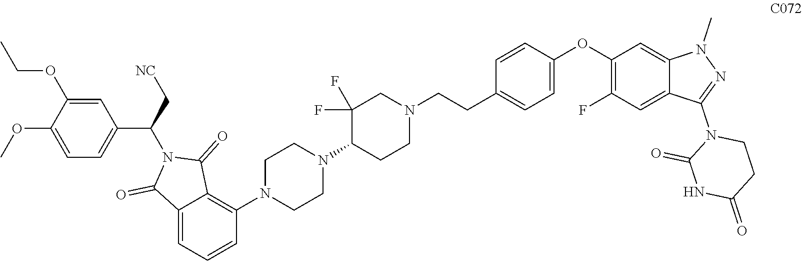 [SMILES: CCOc1cc([C@@H](CC#N)N2C(=O)c3cccc(N4CCN([C@H]5CCN(CCc6ccc(Oc7cc8c(cc7F)c(N7CCC(=O)NC7=O)nn8C)cc6)CC5(F)F)CC4)c3C2=O)ccc1OC]

(R)-3-(6-((R)-1′-(4-((3-(2,4-Dioxotetrahydropyrimidin-1(2H)-yl)-1-methyl-1H-indazol-6-yl)oxy)benzyl)-3′,3′-difluoro-[4,4′-bipiperidin]-1-yl)-1,3-dioxo-1,3-dihydro-2H-pyrrolo[3,4-c]pyridin-2-yl)-3-(3-ethoxy-4-methoxyphenyl)propanenitrile C101.

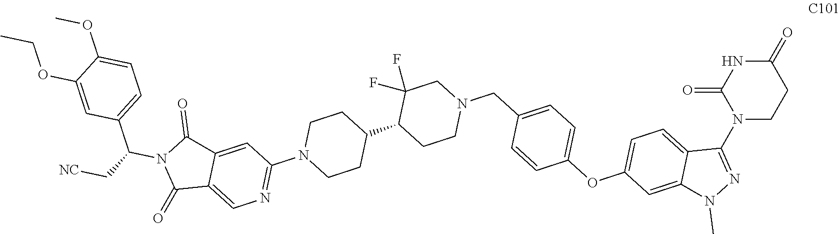 [SMILES: CCOc1cc([C@@H](CC#N)N2C(=O)c3cnc(N4CCC([C@H]5CCN(Cc6ccc(Oc7ccc8c(N9CCC(=O)NC9=O)nn(C)c8c7)cc6)CC5(F)F)CC4)cc3C2=O)ccc1OC]

(R)-3-(6-((R)-1′-(4-((3-(2,4-Dioxotetrahydropyrimidin-1(2H)-yl)-5-fluoro-1-methyl-1H-indazol-6-yl)oxy)benzyl)-3′,3′-difluoro-[4,4′-bipiperidin]-1-yl)-1,3-dioxo-1,3-dihydro-2H-pyrrolo[3,4-c]pyridin-2-yl)-3-(3-ethoxy-4-methoxyphenyl)propanenitrile C102.

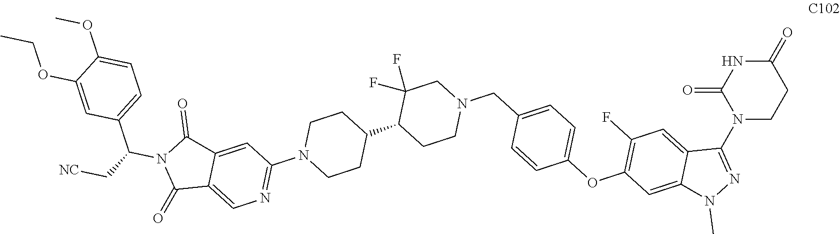 [SMILES: CCOc1cc([C@@H](CC#N)N2C(=O)c3cnc(N4CCC([C@H]5CCN(Cc6ccc(Oc7cc8c(cc7F)c(N7CCC(=O)NC7=O)nn8C)cc6)CC5(F)F)CC4)cc3C2=O)ccc1OC]

(R)-3-(6-(4-((S)-1-(4-((3-(2,4-Dioxotetrahydropyrimidin-1(2H)-yl)-1-methyl-1H-indazol-6-yl)oxy)benzyl)-3,3-difluoropiperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C103.

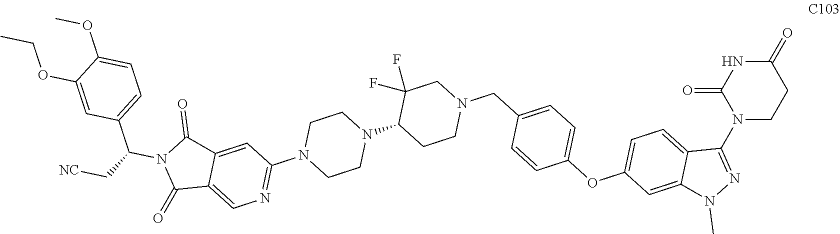 [SMILES: CCOc1cc([C@@H](CC#N)N2C(=O)c3cnc(N4CCN([C@H]5CCN(Cc6ccc(Oc7ccc8c(N9CCC(=O)NC9=O)nn(C)c8c7)cc6)CC5(F)F)CC4)cc3C2=O)ccc1OC]

(R)-3-(6-(4-((S)-1-(4-((3-(2,4-Dioxotetrahydropyrimidin-1(2H)-yl)-5-fluoro-1-methyl-1H-indazol-6-yl)oxy)benzyl)-3,3-difluoropiperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C104.

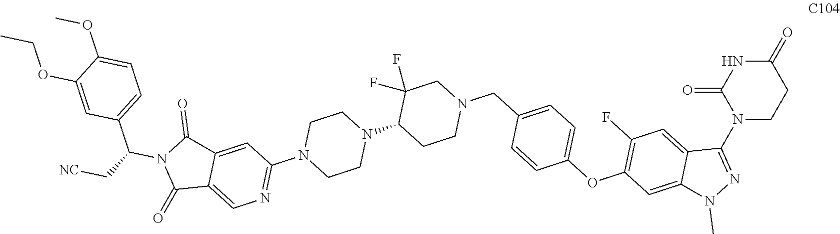 [SMILES: CCOc1cc([C@@H](CC#N)N2C(=O)c3cnc(N4CCN([C@H]5CCN(Cc6ccc(Oc7cc8c(cc7F)c(N7CCC(=O)NC7=O)nn8C)cc6)CC5(F)F)CC4)cc3C2=O)ccc1OC]

(R)-3-(6-(4-((R)-1-(4-((3-(2,4-Dioxotetrahydropyrimidin-1(2H)-yl)-1-methyl-1H-indazol-6-yl)oxy)benzyl)-3,3-difluoropiperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C105.

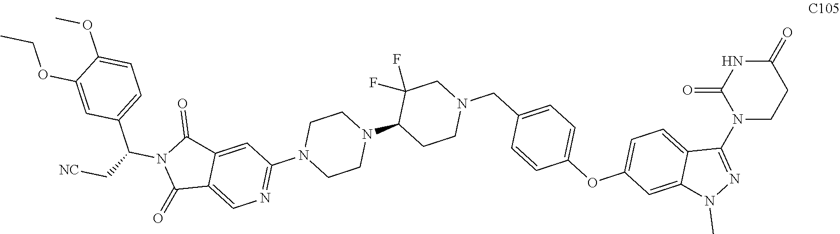 [SMILES: CCOc1cc([C@@H](CC#N)N2C(=O)c3cnc(N4CCN([C@@H]5CCN(Cc6ccc(Oc7ccc8c(N9CCC(=O)NC9=O)nn(C)c8c7)cc6)CC5(F)F)CC4)cc3C2=O)ccc1OC]

(R)-3-(6-(4-((R)-1-(4-((3-(2,4-Dioxotetrahydropyrimidin-1(2H)-yl)-5-fluoro-1-methyl-1H-indazol-6-yl)oxy)benzyl)-3,3-difluoropiperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C106.

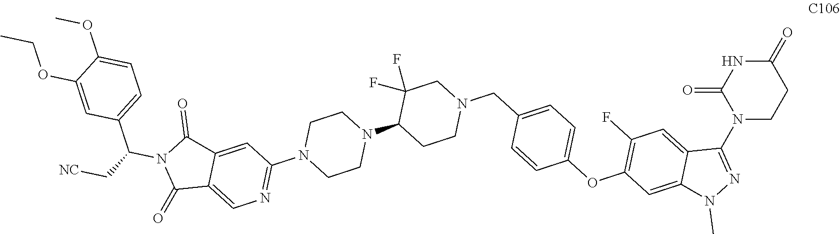 [SMILES: CCOc1cc([C@@H](CC#N)N2C(=O)c3cnc(N4CCN([C@@H]5CCN(Cc6ccc(Oc7cc8c(cc7F)c(N7CCC(=O)NC7=O)nn8C)cc6)CC5(F)F)CC4)cc3C2=O)ccc1OC]

(R)-3-(4-((R)-1′-(4-((3-(2,4-Dioxotetrahydropyrimidin-1(2H)-yl)-1-methyl-1H-indazol-6-yl)oxy)benzyl)-3′,3′-difluoro-[4,4′-bipiperidin]-1-yl)-1,3-dioxo-1,3-dihydro-2H-pyrrolo[3,4-c]pyridin-2-yl)-3-(3-ethoxy-4-methoxyphenyl)propanenitrile C107.

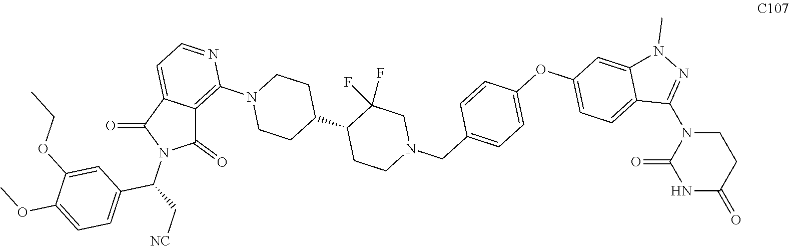 [SMILES: CCOc1cc([C@@H](CC#N)N2C(=O)c3ccnc(N4CCC([C@H]5CCN(Cc6ccc(Oc7ccc8c(N9CCC(=O)NC9=O)nn(C)c8c7)cc6)CC5(F)F)CC4)c3C2=O)ccc1OC]

(R)-3-(4-((R)-1′-(4-((3-(2,4-Dioxotetrahydropyrimidin-1(2H)-yl)-5-fluoro-1-methyl-1H-indazol-6-yl)oxy)benzyl)-3′,3′-difluoro-[4,4′-bipiperidin]-1-yl)-1,3-dioxo-1,3-dihydro-2H-pyrrolo[3,4-c]pyridin-2-yl)-3-(3-ethoxy-4-methoxyphenyl)propanenitrile C108.

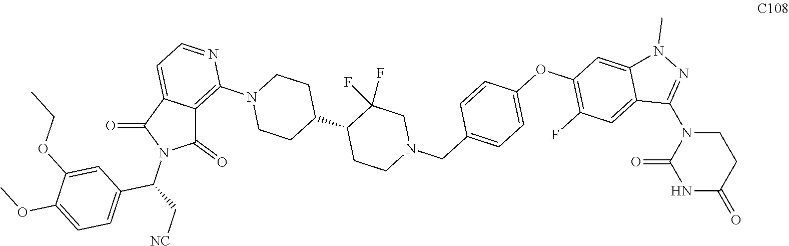 [SMILES: CCOc1cc([C@@H](CC#N)N2C(=O)c3ccnc(N4CCC([C@H]5CCN(Cc6ccc(Oc7cc8c(cc7F)c(N7CCC(=O)NC7=O)nn8C)cc6)CC5(F)F)CC4)c3C2=O)ccc1OC]

(R)-3-(4-(4-((S)-1-(4-((3-(2,4-Dioxotetrahydropyrimidin-1(2H)-yl)-1-methyl-1H-indazol-6-yl)oxy)benzyl)-3,3-difluoropiperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C109.

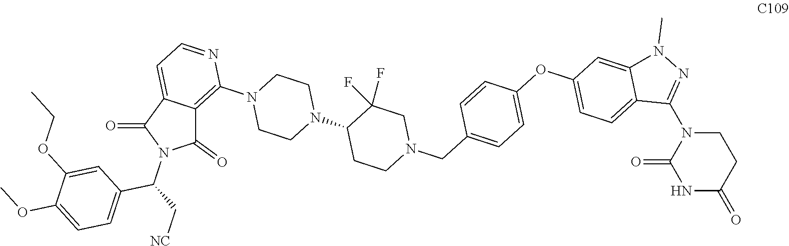 [SMILES: CCOc1cc([C@@H](CC#N)N2C(=O)c3ccnc(N4CCN([C@H]5CCN(Cc6ccc(Oc7ccc8c(N9CCC(=O)NC9=O)nn(C)c8c7)cc6)CC5(F)F)CC4)c3C2=O)ccc1OC]

(R)-3-(4-(4-((S)-1-(4-((3-(2,4-Dioxotetrahydropyrimidin-1(2H)-yl)-5-fluoro-1-methyl-1H-indazol-6-yl)oxy)benzyl)-3,3-difluoropiperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C110.

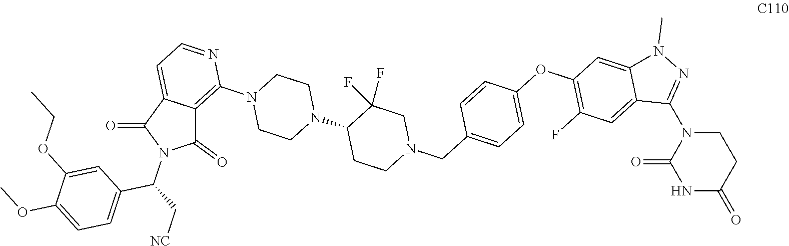 [SMILES: CCOc1cc([C@@H](CC#N)N2C(=O)c3ccnc(N4CCN([C@H]5CCN(Cc6ccc(Oc7cc8c(cc7F)c(N7CCC(=O)NC7=O)nn8C)cc6)CC5(F)F)CC4)c3C2=O)ccc1OC]

(R)-3-(4-(4-((R)-1-(4-((3-(2,4-Dioxotetrahydropyrimidin-1(2H)-yl)-1-methyl-1H-indazol-6-yl)oxy)benzyl)-3,3-difluoropiperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C111.

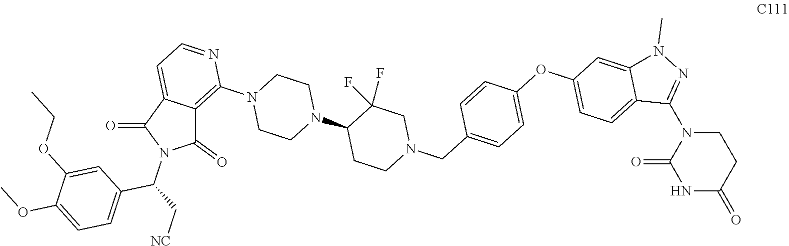 [SMILES: CCOc1cc([C@@H](CC#N)N2C(=O)c3ccnc(N4CCN([C@@H]5CCN(Cc6ccc(Oc7ccc8c(N9CCC(=O)NC9=O)nn(C)c8c7)cc6)CC5(F)F)CC4)c3C2=O)ccc1OC]

(R)-3-(4-(4-((R)-1-(4-((3-(2,4-Dioxotetrahydropyrimidin-1(2H)-yl)-5-fluoro-1-methyl-1H-indazol-6-yl)oxy)benzyl)-3,3-difluoropiperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C112.

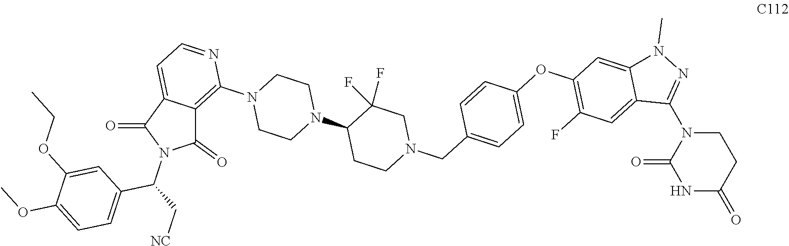 [SMILES: CCOc1cc([C@@H](CC#N)N2C(=O)c3ccnc(N4CCN([C@@H]5CCN(Cc6ccc(Oc7cc8c(cc7F)c(N7CCC(=O)NC7=O)nn8C)cc6)CC5(F)F)CC4)c3C2=O)ccc1OC]

(R)-3-(4-(4-(I-(2-(4-((3-(2,4-Dioxotetrahydropyrimidin-1(2H)-yl)-1-methyl-1H-indazol-6-yl)oxy)phenyl)-2,2-difluoroethyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C113.

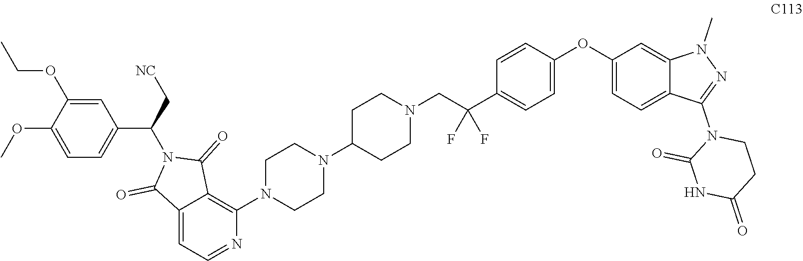 [SMILES: CCOc1cc([C@@H](CC#N)N2C(=O)c3ccnc(N4CCN(C5CCN(CC(F)(F)c6ccc(Oc7ccc8c(N9CCC(=O)NC9=O)nn(C)c8c7)cc6)CC5)CC4)c3C2=O)ccc1OC]

(R)-3-(4-(4-(1-(2-(4-((3-(2,4-Dioxotetrahydropyrimidin-1(2H)-yl)-5-fluoro-1-methyl-1H-indazol-6-yl)oxy)phenyl)-2,2-difluoroethyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C114.

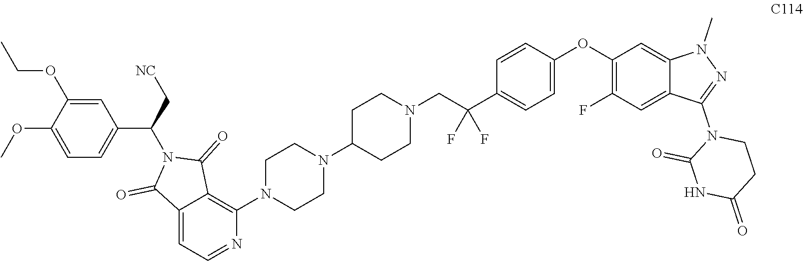 [SMILES: CCOc1cc([C@@H](CC#N)N2C(=O)c3ccnc(N4CCN(C5CCN(CC(F)(F)c6ccc(Oc7cc8c(cc7F)c(N7CCC(=O)NC7=O)nn8C)cc6)CC5)CC4)c3C2=O)ccc1OC]

(R)-3-(4-(4-(2-(4-(4-((3-(2,4-Dioxotetrahydropyrimidin-1(2H)-yl)-1-methyl-1H-indazol-6-yl)oxy)phenyl)piperazin-1-yl)ethyl)piperidin-1-yl)-1,3-dioxo-1,3-dihydro-2H-pyrrolo-[3,4-c]pyridin-2-yl)-3-(3-ethoxy-4-methoxyphenyl)propanenitrile C115.

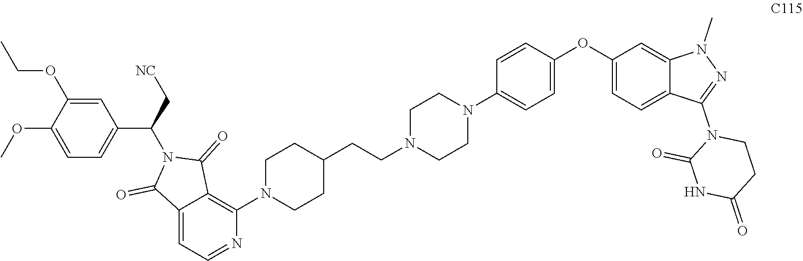 [SMILES: CCOc1cc([C@@H](CC#N)N2C(=O)c3ccnc(N4CCC(CCN5CCN(c6ccc(Oc7ccc8c(N9CCC(=O)NC9=O)nn(C)c8c7)cc6)CC5)CC4)c3C2=O)ccc1OC]

(R)-3-(4-(4-(2-(4-(4-((3-(2,4-Dioxotetrahydropyrimidin-1(2H)-yl)-5-fluoro-1-methyl-1H-indazol-6-yl)oxy)phenyl)piperazin-1-yl)ethyl)piperidin-1-yl)-1,3-dioxo-1,3-dihydro-2H-pyrrolo[3,4-c]pyridin-2-yl)-3-(3-ethoxy-4-methoxyphenyl)propanenitrile C116.

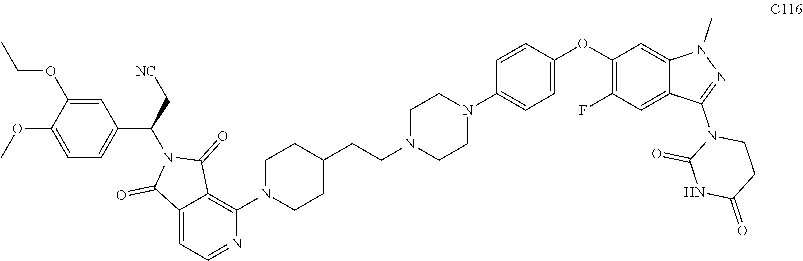 [SMILES: CCOc1cc([C@@H](CC#N)N2C(=O)c3ccnc(N4CCC(CCN5CCN(c6ccc(Oc7cc8c(cc7F)c(N7CCC(=O)NC7=O)nn8C)cc6)CC5)CC4)c3C2=O)ccc1OC]

(R)-3-(4-(4-(2-(1-(4-((3-(2,4-Dioxotetrahydropyrimidin-1(2H)-yl)-1-methyl-1H-indazol-6-yl)oxy)phenyl)piperidin-4-yl)ethyl)piperazin-1-yl)-1,3-dioxo-1,3-dihydro-2H-pyrrolo-[3,4-c]pyridin-2-yl)-3-(3-ethoxy-4-methoxyphenyl)propanenitrile C117.

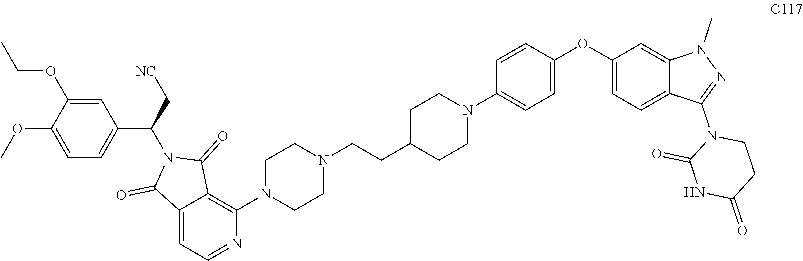 [SMILES: CCOc1cc([C@@H](CC#N)N2C(=O)c3ccnc(N4CCN(CCC5CCN(c6ccc(Oc7ccc8c(N9CCC(=O)NC9=O)nn(C)c8c7)cc6)CC5)CC4)c3C2=O)ccc1OC]

(R)-3-(4-(4-(2-(1-(4-((3-(2,4-Dioxotetrahydropyrimidin-1(2H)-yl)-5-fluoro-1-methyl-1H-indazol-6-yl)oxy)phenyl)piperidin-4-yl)ethyl)piperazin-1-yl)-1,3-dioxo-1,3-dihydro-2H-pyrrolo[3,4-c]pyridin-2-yl)-3-(3-ethoxy-4-methoxyphenyl)propanenitrile C118.

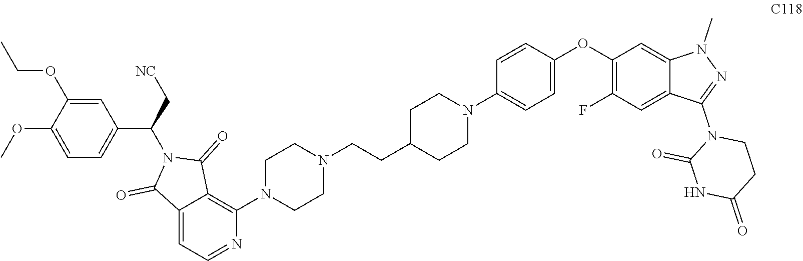 [SMILES: CCOc1cc([C@@H](CC#N)N2C(=O)c3ccnc(N4CCN(CCC5CCN(c6ccc(Oc7cc8c(cc7F)c(N7CCC(=O)NC7=O)nn8C)cc6)CC5)CC4)c3C2=O)ccc1OC]

5-(5-(4-(1-(2,6-Dioxopiperidin-3-yl)indolin-6-yl)-[1,4′-bipiperidin]-1′-yl)pentyl)-2-((S)-1-(3-ethoxy-4-methoxyphenyl)-2-(methylsulfonyl)ethyl)isoindoline-1,3-dione D001.

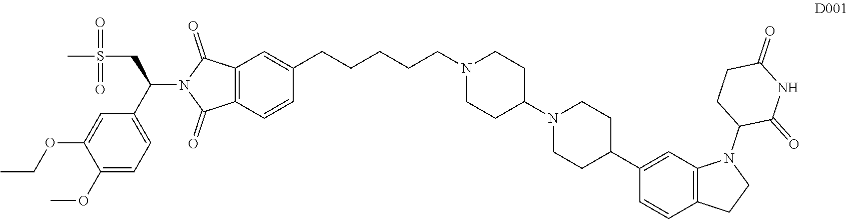 [SMILES: CCOc1cc([C@@H](CS(C)(=O)=O)N2C(=O)c3ccc(CCCCCN4CCC(N5CCC(c6ccc7c(c6)N(C6CCC(=O)NC6=O)CC7)CC5)CC4)cc3C2=O)ccc1OC]

5-(4-((1′-(3-(2,6-Dioxopiperidin-3-yl)-1-methyl-2-oxo-2,3-dihydro-1H-benzo[d]-imidazol-5-yl)-[4,4′-bipiperidin]-1-yl)methyl)piperidin-1-yl)-2-((S)-1-(3-ethoxy-4-methoxy-phenyl)-2-(methylsulfonyl)ethyl)isoindoline-1,3-dione D002.

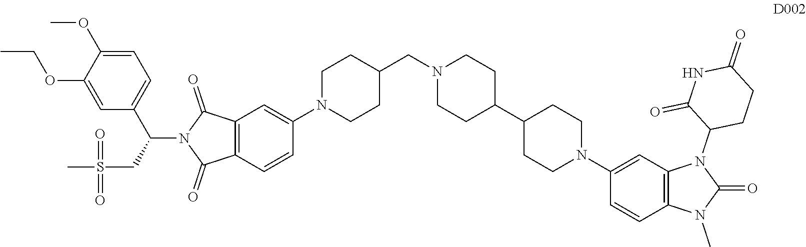 [SMILES: CCOc1cc([C@@H](CS(C)(=O)=O)N2C(=O)c3ccc(N4CCC(CN5CCC(C6CCN(c7ccc8c(c7)n(C7CCC(=O)NC7=O)c(=O)n8C)CC6)CC5)CC4)cc3C2=O)ccc1OC]

5-((1-((1-((1-(3-(2,6-Dioxopiperidin-3-yl)-1-methyl-2-oxo-2,3-dihydro-1H-benzo[d]imidazol-5-yl)azetidin-3-yl)methyl)piperidin-4-yl)methyl)piperidin-4-yl)amino)-2-((S)-1-(3-ethoxy-4-methoxyphenyl)-2-(methylsulfonyl)ethyl)isoindoline-1,3-dione D003.

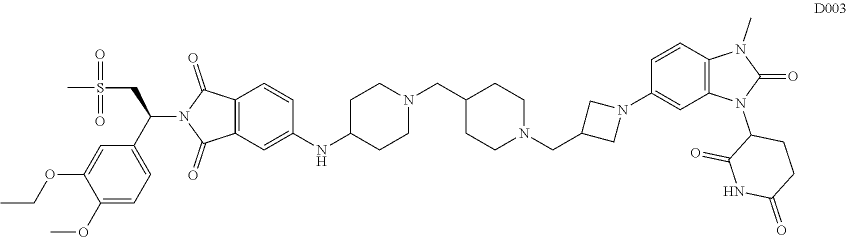 [SMILES: CCOc1cc([C@@H](CS(C)(=O)=O)N2C(=O)c3ccc(NC4CCN(CC5CCN(CC6CN(c7ccc8c(c7)n(C7CCC(=O)NC7=O)c(=O)n8C)C6)CC5)CC4)cc3C2=O)ccc1OC]

### Example B1. Cell-based Cytokine Inhibition Assay

Frozen primary blood mononuclear cells (PBMCs) were thawed and washed once with RPMI 1640 media supplemented with 10% fetal bovine serum, 1% penicillin, and 1% streptomycin. An anti-CD28 antibody at 1 mg/mL was added to the media as a co-stimulant. PBMCs were plated at 1×10⁶ cells per well in a 96-well U bottom plate, which was pre-coated overnight with an anti-CD3 antibody at 1 μg/mL in PBS (100 μL/well). After the place was incubated for 1 h at 37° C. in a CO₂ incubator, DMSO (control) or a compound was added. After the place was incubated for 23 h at 37° C. in a CO₂ incubator, the cells were spun down at 1,500 rpm for 5 min. Supernatant media was collected for cytokine analysis using an MSD kit. Inhibitory activities of the compound were determined as a percentage of the DMSO control. The results are summarized in Table 1.

TABLE 1
Cytokine Inhibition

| | IC₅₀ (nM) | | | |
|---|---|---|---|---|
| Cmpd. | TNFα | IFNγ | IL-2 | IL-10 |
| B001 | 0.3 | 0.9 | 0.2 | 0.3 |
| B002 | 0.09 | 0.3 | 0.09 | 0.2 |

### Example B2. Protein Degradation Assay

A549 cells were grown in RPMI 1640 media supplemented with 10% fetal bovine serum, streptomycin, and penicillin. The cells were plated in 6-well plates at 300,000 cells/well in the growth media. After overnight incubation, the cells were treated with DMSO (control) or a compound at predetermined concentrations (e.g., 10 nM, 100 nM, 1 μM, 3 μM, or 10 μM) for 24 h at 37° C. under 5% CO₂. Whole cell extracts were prepared using an immunoprecipitation (IP) lysis buffer. The cells were washed once in PBS, and the cell pellets were resuspended in the IP lysis buffer and incubated for 15 min on ice. Cells debris was removed by centrifugation and the cleared whole cell lysates were transferred to new tubes for further analysis.

For a western blot analysis, the whole cell protein extracts were separated on 4-12% SDS-polyacrylamide gels, transferred to nitrocellulose, and probed with primary antibodies. Membranes were subsequently washed and probed with IRDYE® secondary antibodies. The signals were detected using an ODYSSEY® Imaging System. The antibodies used in the assay included anti-PDE4B antibody; anti-PDE4D antibodies (top, bottom, and short isoform); β-actin mouse monoclonal antibody; IRDYE® 680RD goat anti-rabbit antibody; and IRDYE® 800CW goat anti-mouse antibody. Compounds B001, B002, B003, and B007 were determined to be able to degradate PDE4B as high as about 70% relative to DMSO; and PDE4D, in particular, PDE4D short isoform, as high as about 95% relative to DMSO; whereas apremilast did not degrade the PDE4D under the same conditions.

The examples set forth above are provided to give those of ordinary skill in the art with a complete disclosure and description of how to make and use the claimed embodiments and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed is:

1. A compound of Formula (A): 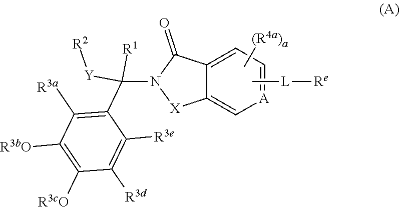
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof, wherein:
A is C(R⁴) or N;
L is a linker;
X is —CH₂— or —C(O)—;
Y is C₁₋₆ alkylene or C₃₋₁₀ cycloalkylene;
R¹ is hydrogen or deuterium;
R² is cyano or —S(O₂)R^{2a};
R⁴ is hydrogen or R^{4a};
R^{2a} is (i) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (ii) —OR^{1a} or —NR^{1b}R^{1c};
R^{3a}, R^{3d}, and R^{3e} are each independently (i) hydrogen, deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{3b} and R^{3c} are each independently C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
each R^{4a} is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{e} is 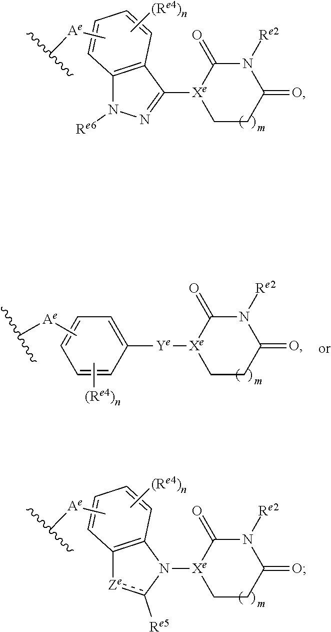
wherein:
each A^{e} is independently (i) C₁₋₆ alkylene, C₂₋₆ alkenylene, C₂₋₆ alkynylene, C₃₋₁₀ cycloalkylene, C₆₋₁₄ arylene, heteroarylene, or heterocyclylene;
or (ii) —O— or —NHCH₂CH₂—;
X^{e} is C(R^{e1}) or N;
Y^{e} is a bond, C₁₋₆ alkylene, —O—, —S—, —S(O)—, —S(O₂)—, or —N(R^{e3})—;
Z^{e} is —CH═, —CH₂—, —N═, or —N(R^{e6})—;
R^{e1} is hydrogen, deuterium, halo, or C₁₋₆ alkyl;
each R^{e2}, R^{e3}, and R^{e6} is independently hydrogen or C₁₋₆ alkyl;
each R^{e4} is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{e5} is hydrogen or oxo;
each m is independently an integer of 0, 1, or 2; and
each n is independently an integer of 0, 1, 2, 3, or 4;
each R^{1a}, R^{1b}, R^{1e}, and R^{1d} is independently hydrogen, deuterium, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; and
a is an integer of 0, 1, 2, 3, or 4;
wherein each alkyl, alkylene, heteroalkyl, alkenyl, alkenylene, alkynyl, alkynylene, cycloalkyl, cycloalkylene, aryl, arylene, aralkyl, heteroaryl, heteroarylene, heterocyclyl, and heterocyclylene is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{a})OR^{d}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NRS(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OP(O(OR^{f})OR^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

2. The compound of claim 1, having the structure of Formula (IA): 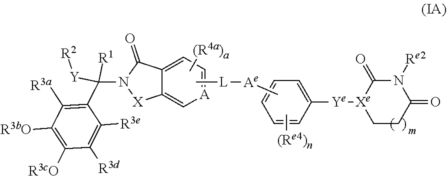
or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof.

3. The compound of claim 1, having the structure of Formula (IB): 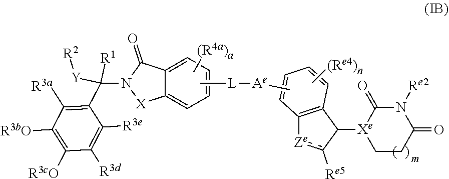
or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof.

4. The compound of claim 1, having the structure of Formula (IC): 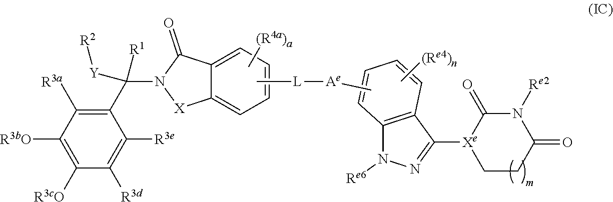
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

5. The compound of claim 1, having the structure of Formula (ID): 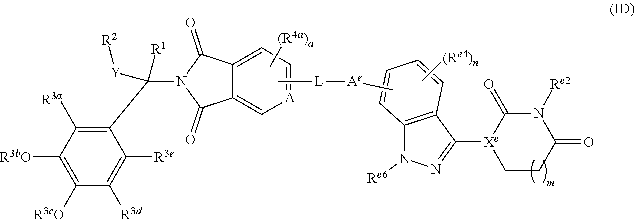
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

6. The compound of any one of claims 1 to 5, wherein Y is C₁₋₆ alkylene, optionally substituted with one or more substituents Q.

7. The compound of any one of claims 1, 5, and 6, having the structure of Formula (IID): 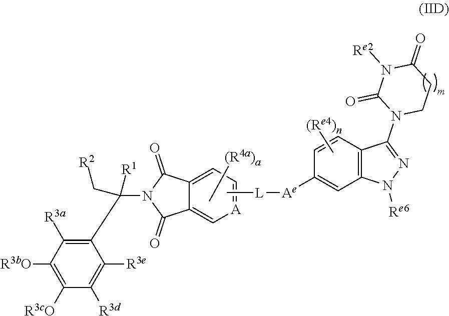
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

8. The compound of any one of claims 1 and 5 to 7, having the structure of Formula (VD): 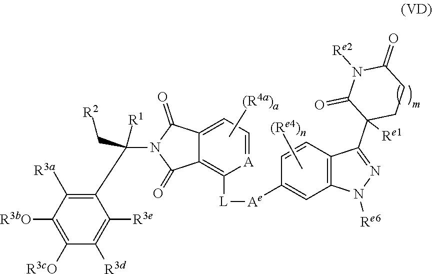
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

9. The compound of any one of claims 1 and 5 to 7, having the structure of Formula (VID): 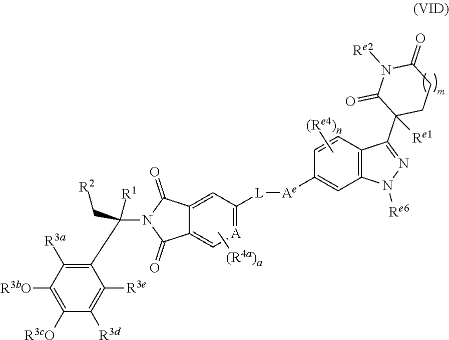
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

10. The compound of any one of claims 1, 5, and 6, having the structure of Formula (VIID): 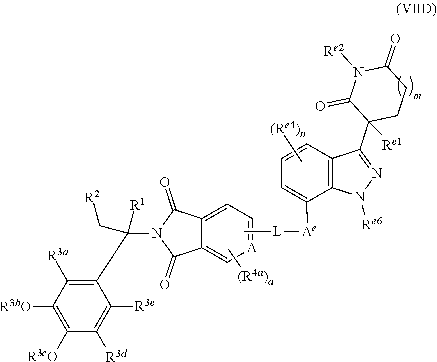
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

11. The compound of any one of claims 1, 5, 6, and 10, having the structure of Formula (XD): 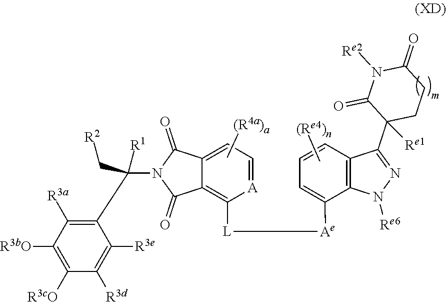
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

12. The compound of any one of claims 1, 5, 6, and 10, having the structure of Formula (XID): 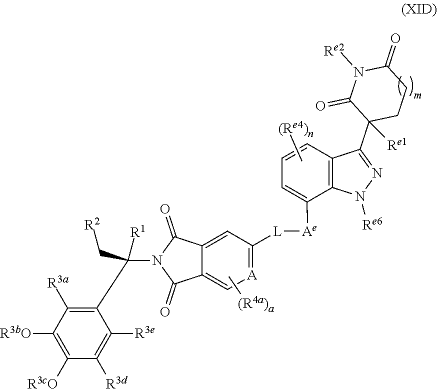
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

13. The compound of any one of claims 7 to 12, wherein R^{e1} is (i) hydrogen, deuterium, or halo; or (ii) C₁₋₆ alkyl, optionally substituted with one or more substituents Q.

14. The compound of any one of claims 7 to 13, wherein R^{e1} is hydrogen, deuterium, fluoro, or methyl.

15. The compound of any one of claims 7 to 14, wherein R^{e1} is hydrogen.

16. The compound of any one of claims 1, 5, and 6, having the structure of Formula (XIID): 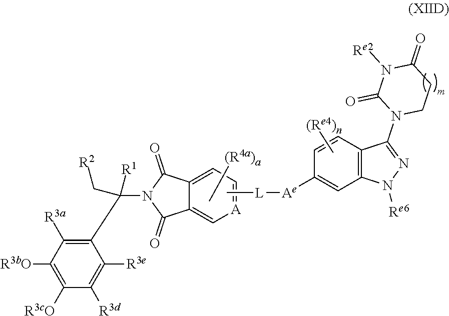
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

17. The compound of any one of claims 1, 5, 6, and 16, having the structure of Formula (XVD): 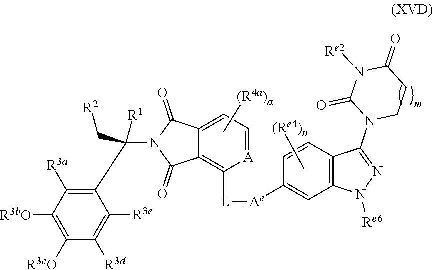
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

18. The compound of any one of claims 1, 5, 6, and 16, having the structure of Formula (XVID): 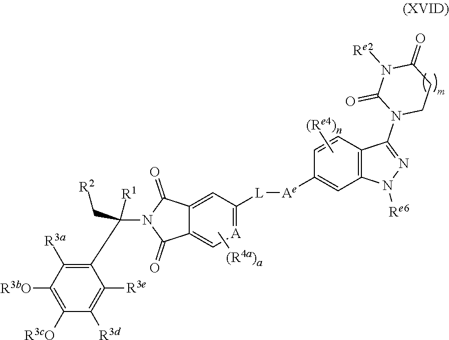
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

19. The compound of any one of claims 1, 5, and 6, having the structure of Formula (XVIID): 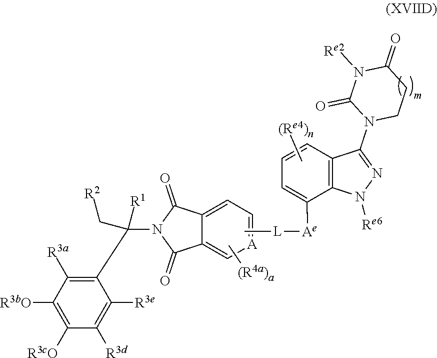
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

20. The compound of any one of claims 1, 5, 6, and 19, having the structure of Formula (XXD): 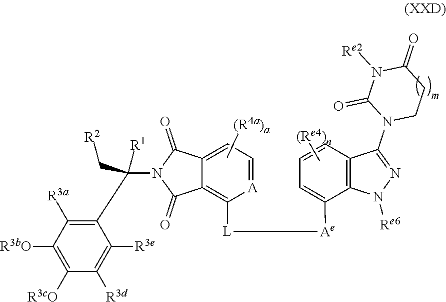
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

21. The compound of any one of claims 1, 5, 6, and 19, having the structure of Formula (XXID): 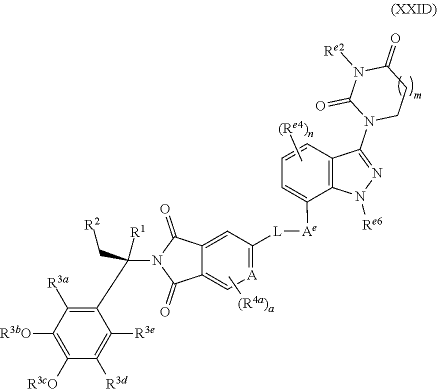
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

22. The compound of any one of claims 1 to 21, wherein R¹ is hydrogen.

23. The compound of any one of claims 1 to 22, wherein R² is cyano.

24. The compound of any one of claims 1 to 22, wherein R² is —S(O₂)R^{2a}.

25. The compound of any one of claims 1 to 22 and 24, wherein R^{2a} is C₁₋₆ alkyl or C₃₋₁₀ cycloalkyl, each optionally substituted with one or more substituents Q.

26. The compound of any one of claims 1 to 22, 24, and 25, wherein R^{2a} is C₁₋₆ alkyl, optionally substituted with one or more substituents Q.

27. The compound of any one of claims 1 to 22, 24, and 25, wherein R^{2a} is C₃₋₁₀ cycloalkyl, optionally substituted with one or more substituents Q.

28. The compound of any one of claims 1 to 22, 24, and 25, wherein R^{2a} is methyl, cyclopropylmethyl, or cyclopropyl.

29. The compound of any one of claims 1 to 22, 24, 25, and 28, wherein R^{2a} is methyl.

30. The compound of any one of claims 1 to 29, wherein R^{3a} is hydrogen.

31. The compound of any one of claims 1 to 30, wherein R^{3b} is C₁₋₆ alkyl or C₃₋₁₀ cycloalkyl, each optionally substituted with one or more substituents Q.

32. The compound of any one of claims 1 to 31, wherein R^{3b} is C₁₋₆ alkyl, optionally substituted with one or more substituents Q.

33. The compound of any one of claims 1 to 31, wherein R^{3b} is C₃₋₁₀ cycloalkyl, optionally substituted with one or more substituents Q.

34. The compound of any one of claims 1 to 31, wherein R^{3b} is methyl, ethyl, cyclopropylmethyl, or cyclopropyl.

35. The compound of any one of claims 1 to 32 and 34, wherein R^{3b} is methyl or ethyl.

36. The compound of any one of claims 1 to 32, 34, and 35, wherein R^{3b} is ethyl.

37. The compound of any one of claims 1 to 36, wherein R^{3c} is C₁₋₆ alkyl or C₃₋₁₀ cycloalkyl, each optionally substituted with one or more substituents Q.

38. The compound of any one of claims 1 to 37, wherein R^{3c} is C₁₋₆ alkyl, optionally substituted with one or more substituents Q.

39. The compound of any one of claims 1 to 37, wherein R^{3c} is C₃₋₁₀ cycloalkyl, optionally substituted with one or more substituents Q.

40. The compound of any one of claims 1 to 37, wherein R^{3c} is methyl, ethyl, cyclopropylmethyl, or cyclopropyl.

41. The compound of any one of claims 1 to 38 and 40, wherein R^{3c} is methyl or ethyl.

42. The compound of any one of claims 1 to 38, 40, and 41, wherein R^{3c} is methyl.

43. The compound of any one of claims 1 to 42, wherein R^{3d} is hydrogen.

44. The compound of any one of claims 1 to 43, wherein R^{3e} is hydrogen.

45. The compound of any one of claims 1 to 44, wherein a is an integer of 1.

46. The compound of any one of claims 1 to 45, wherein R^{4a} is deuterium, halo, —NR^{1b}R^{1c}, or —NR^{1a}C(O)R^{1d}.

47. The compound of any one of claims 1 to 46, wherein R^{4a} is deuterium, fluoro, chloro, —NH₂, or —NHC(O)CH₃.

48. The compound of any one of claims 1 to 47, wherein R^{4a} is —NHC(O)CH₃.

49. The compound of any one of claims 1 to 44, wherein a is an integer of 0.

50. The compound of any one of claims 1 to 49, wherein A is C(R⁴).

51. The compound of any one of claims 1 to 50, wherein A is C(H).

52. The compound of any one of claims 1 to 49, wherein A is N.

53. The compound of any one of claims 1 to 52, wherein A^{e} is —O—.

54. The compound of any one of claims 1 to 53, wherein R^{e2} is hydrogen.

55. The compound of any one of claims 1 to 54, wherein n is an integer of 0, 1, or 2.

56. The compound of any one of claims 1 to 55, wherein n is an integer of 1.

57. The compound of any one of claims 1 to 56, wherein R^{e4} is halo.

58. The compound of any one of claims 1 to 57, wherein R^{e4} is fluoro.

59. The compound of any one of claims 1 to 55, wherein n is an integer of 0.

60. The compound of any one of claims 1 and 4 to 59, wherein R^{e6} is C₁₋₆ alkyl, optionally substituted with one or more substituents Q.

61. The compound of any one of claims 1 and 4 to 59, wherein R^{e6} is methyl.

62. The compound of any one of claims 1 to 61, wherein m is an integer of 1.

63. The compound of any one of claims 1 to 62, wherein L is a linker having the structure of —Zⁿ—(Rⁿ—Zⁿ)_{z}—, wherein:
each Rⁿ is independently C₁₋₆ alkylene, C₂₋₆ alkenylene, C₂₋₆ alkynylene, C₃₋₁₀ cycloalkylene, C₆₋₁₄ arylene, heteroarylene, or heterocyclylene, each of which is optionally substituted with one or more substituents Q;
each Zⁿ is independently a bond, —C(O)—, —C(O)O—, —C(O)NR^{1b}—, —C(O)S—, —C(NR^{1a})NR^{1b}—, —C(S)—, —C(S)O—, —C(S)NR^{1b}—, —O—, —OC(O)O—, —OC(O)NR^{1b}—, —OC(O)S—, —OC(NR^{1a})NR^{1b}—, —OC(S)O—, —OC(S)NR^{1b}—, —OS(O)—, —OS(O)₂, —OS(O)NR^{1b}—, —OS(O)₂NR^{1b}—, —NR^{1b}—, —NR^{1a}C(O)NR^{1b}—, —NR^{1a}C(O)S—, —NR^{1a}C(NR^{1d})NR^{1b}—, —NR^{1a}C(S)NR^{1b}—, —NR^{1a}S(O)NR^{1b}—, —NR^{1a}S(O)₂NR^{1b}—, —S—, —S(O)—, —S(O)₂—, —S(O)NR^{1b}—, or —S(O)₂NR^{1b}—; and
z is an integer of 1, 2, 3, 4, or 5.

64. The compound of claim 63, wherein each Rⁿ is independently C₁₋₆ alkylene, C₆₋₁₄ arylene, or heterocyclylene, each optionally substituted with one or more substituents Q.

65. The compound of claim 63 or 64, wherein each Rⁿ is independently methanediyl, ethane-1,2-diyl, propane-1,3-diyl, butane-1,4-diyl, pentane-1,5-diyl, hexane-1,6-diyl, phen-1,3-diyl, phen-1,4-diyl, azetidin-1,3-diyl, piperidin-1,4-diyl, or piperazin-1,4-diyl, each optionally substituted with one or more substituents Q.

66. The compound of cany one of claims 63 to 65, wherein each Rⁿ is independently methanediyl, ethane-1,2-diyl, phen-1,4-diyl, piperidin-1,4-diyl, or piperazin-1,4-diyl, each optionally substituted with one, two, three, or four fluoro.

67. The compound of any one of claims 63 to 66, wherein each Zⁿ is independently a bond, —C(O)—, —C(O)NR^{1b}—, —O—, or —NR^{1b}—.

68. The compound of any one of claims 63 to 67, wherein each Zⁿ is independently a bond, —C(O)—, —C(O)NH—, —O—, or —NH—.

69. The compound of any one of claims 1 to 68, wherein L is:

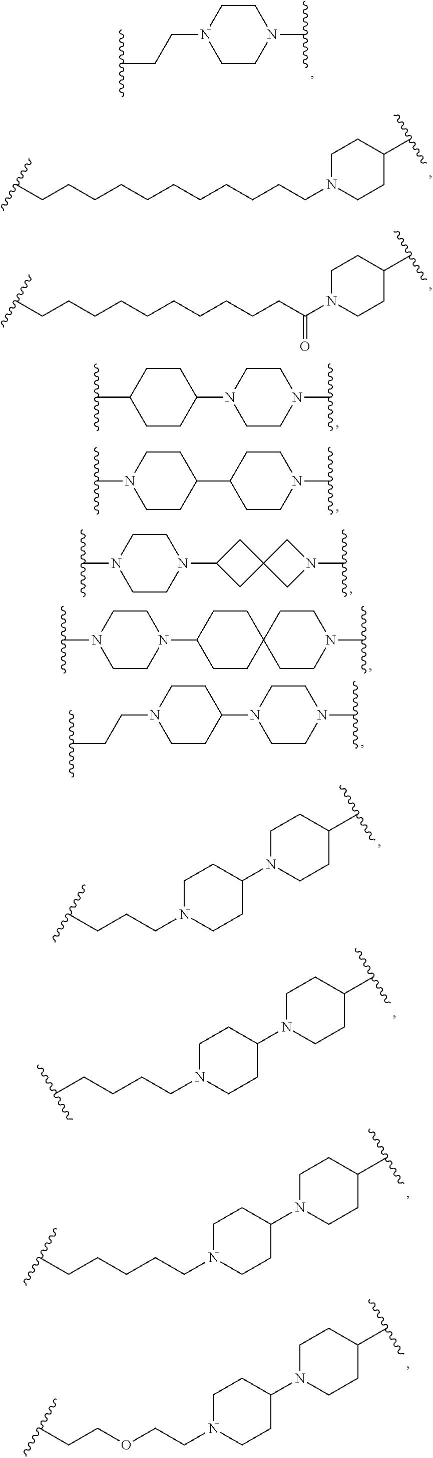

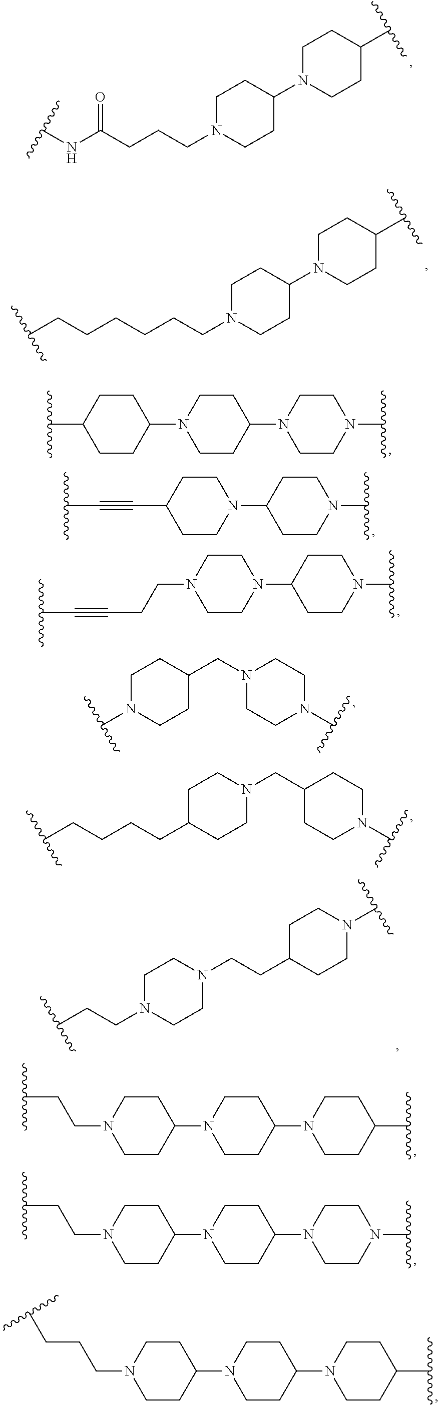

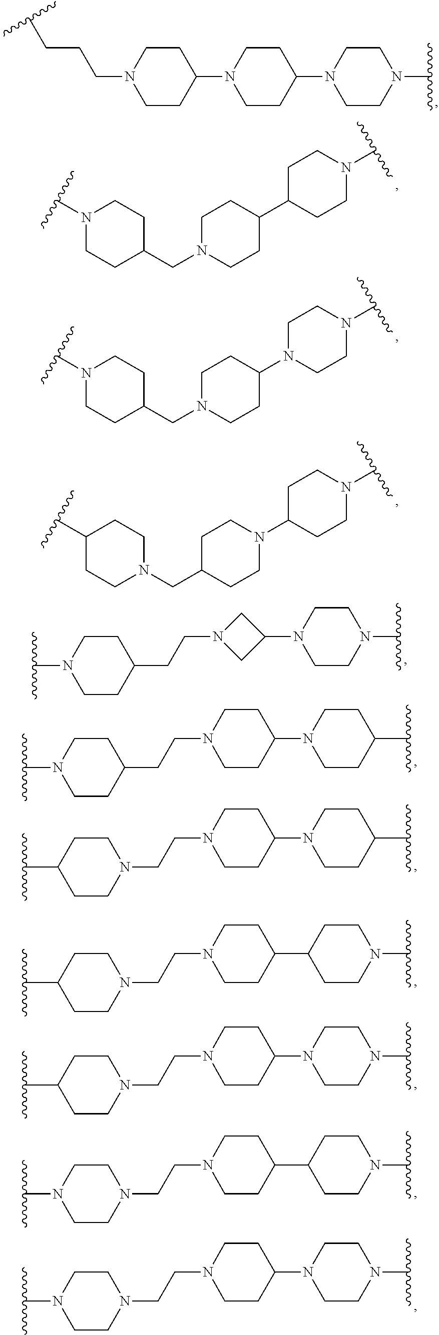

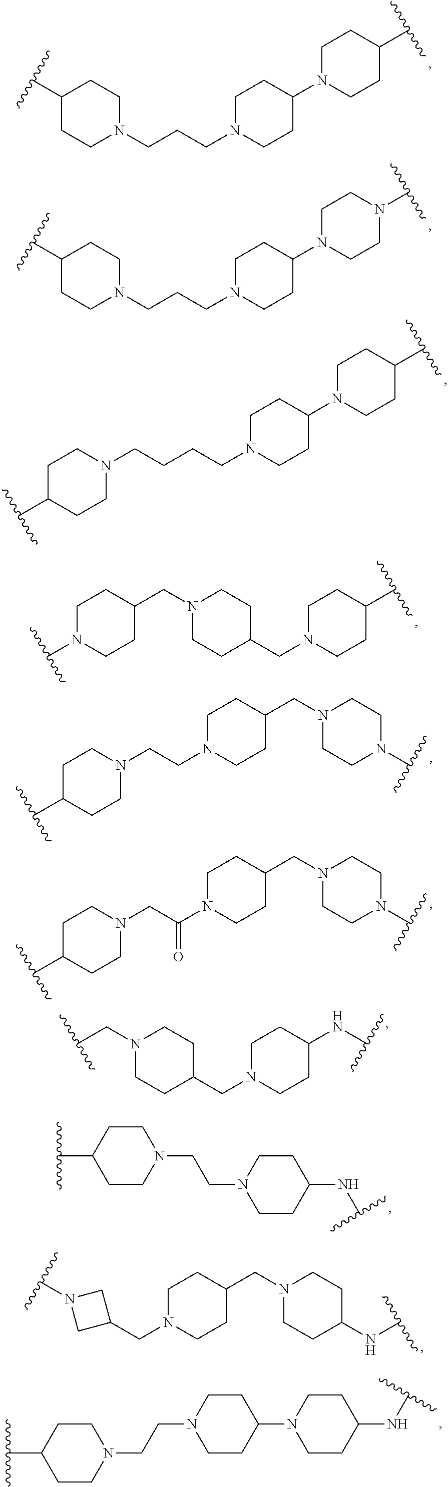

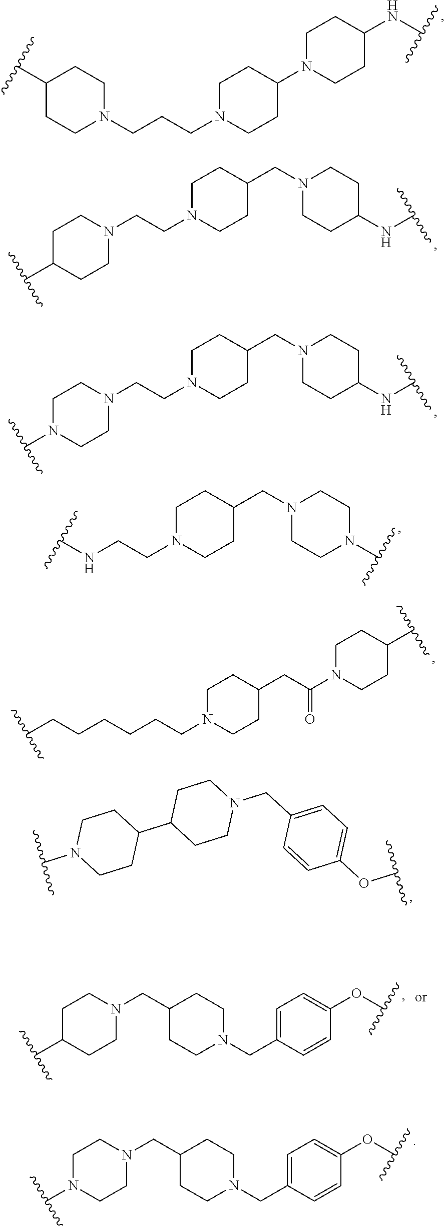

70. The compound of any one of claims 1 to 68, wherein L is: 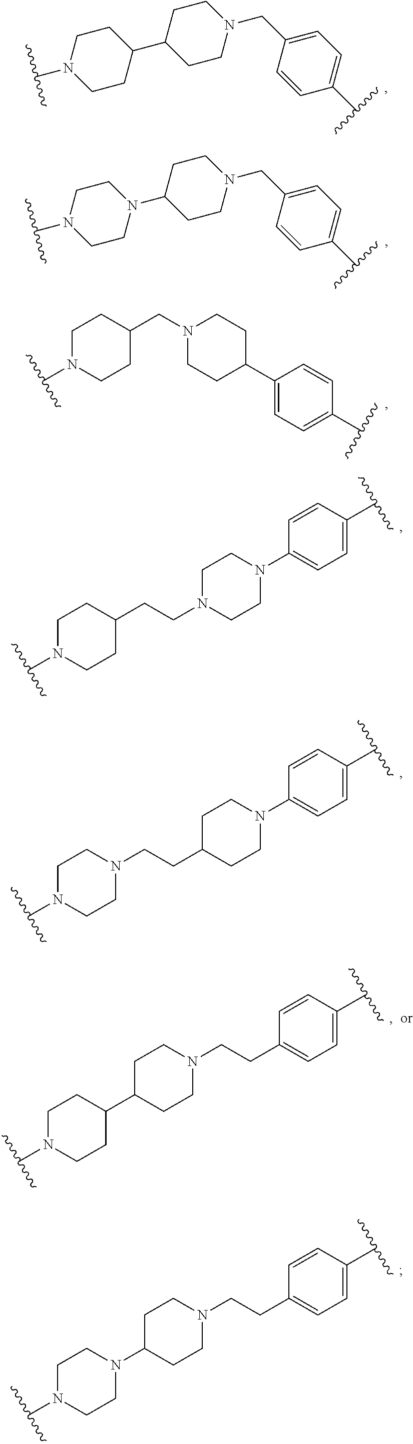
each is optionally substituted with one, two, three, or four fluoro.

71. The compound of any one of claims 1 to 68 and 70, wherein L is:

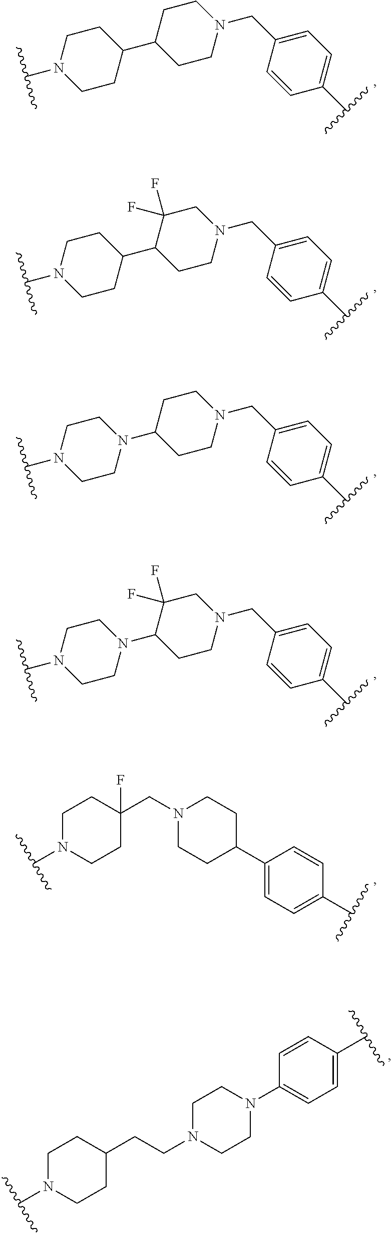 [SMILES: *c1ccc(CN2CCC(C3CCN(*)CC3)CC2)cc1 ; *c1ccc(CN2CCC(C3CCN(*)CC3)C(F)(F)C2)cc1 ; *c1ccc(CN2CCC(N3CCN(*)CC3)CC2)cc1 ; *c1ccc(CN2CCC(N3CCN(*)CC3)C(F)(F)C2)cc1 ; *c1ccc(C2CCN(CC3(F)CCN(*)CC3)CC2)cc1 ; *c1ccc(N2CCN(CCC3CCN(*)CC3)CC2)cc1]

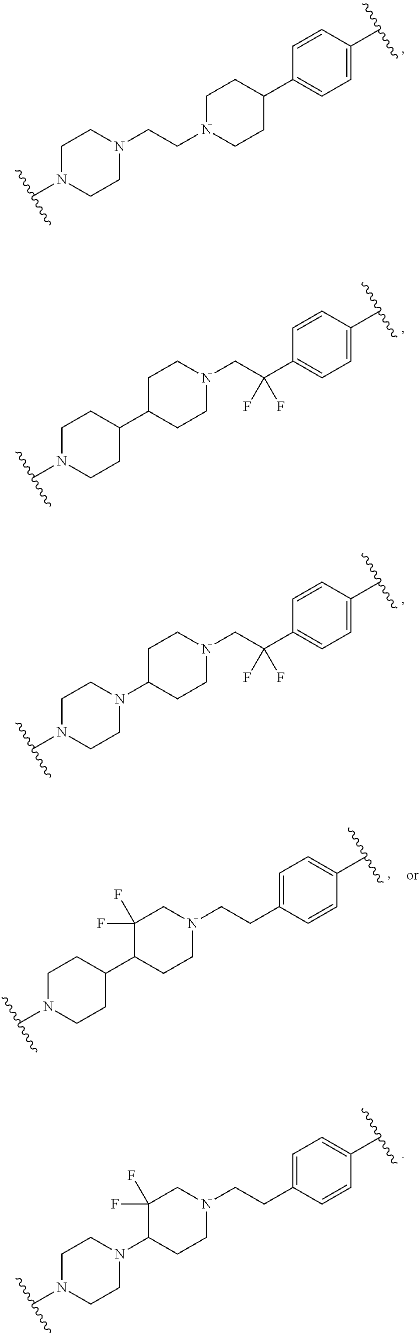 [SMILES: *c1ccc(C2CCN(CCN3CCN(*)CC3)CC2)cc1 ; *c1ccc(C(F)(F)CN2CCC(C3CCN(*)CC3)CC2)cc1 ; *c1ccc(C(F)(F)CN2CCC(N3CCN(*)CC3)CC2)cc1 ; *c1ccc(CCN2CCC(C3CCN(*)CC3)C(F)(F)C2)cc1 ; *c1ccc(CCN2CCC(N3CCN(*)CC3)C(F)(F)C2)cc1]

72. The compound of claim 1, wherein the compound is:
3-(4-(1′-(5-(2-((S)-1-(3-ethoxy-4-methoxyphenyl)-2-(methylsulfonyl)ethyl)-1-oxoisoindolin-5-yl)pentyl)-[1,4′-bipiperidin]-4-yl)phenyl)piperidine-2,6-dione A001;
5-(6-(4-(4-(2,6-dioxopiperidin-3-yl)phenyl)-[1,4′-bipiperidin]-1′-yl)hexyl)-2-((S)-1-(3-ethoxy-4-methoxyphenyl)-2-(methylsulfonyl)ethyl)isoindoline-1,3-dione A002;
3-(4-(1′-(5-(2-((S)-1-(3-ethoxy-4-methoxyphenyl)-2-(methylsulfonyl)ethyl)-3-oxoisoindolin-5-yl)pentyl)-[1,4′-bipiperidin]-4-yl)phenyl)piperidine-2,6-dione A003;
N-(6-(5-(4-(4-(2,6-dioxopiperidin-3-yl)phenyl)-[1,4′-bipiperidin]-1′-yl)pentyl)-2-((S)-1-(3-ethoxy-4-methoxyphenyl)-2-(methylsulfonyl)ethyl)-1,3-dioxoisoindolin-4-yl)-acetamide A004;
5-(5-(4-(3-(2,6-dioxopiperidin-3-yl)phenyl)-[1,4′-bipiperidin]-1′-yl)pentyl)-2-((S)-1-(3-ethoxy-4-methoxyphenyl)-2-(methylsulfonyl)ethyl)isoindoline-1,3-dione A005;
5-(2-(2-(4-(4-(2,6-dioxopiperidin-3-yl)phenyl)-[1,4′-bipiperidin]-1′-yl)ethoxy)-ethyl)-2-((S)-1-(3-ethoxy-4-methoxyphenyl)-2-(methylsulfonyl)ethyl)isoindoline-1,3-dione A006;
5-(4-(1-((1-(4-(2,6-dioxopiperidin-3-yl)phenyl)piperidin-4-yl)methyl)piperidin-4-yl)butyl)-2-((S)-1-(3-ethoxy-4-methoxyphenyl)-2-(methylsulfonyl)ethyl)isoindoline-1,3-dione A007;
4-(3-(4-(4-(2,6-dioxopiperidin-3-yl)phenyl)-[1,4′:1′,4″-terpiperidin]-1″-yl)-propyl)-2-((S)-1-(3-ethoxy-4-methoxyphenyl)-2-(methylsulfonyl)ethyl)isoindoline-1,3-dione A008;
5-(3-(4-(4-(2,6-dioxopiperidin-3-yl)phenyl)-[1,4′:1′,4″-terpiperidin]-1″-yl)-propyl)-2-((S)-1-(3-ethoxy-4-methoxyphenyl)-2-(methylsulfonyl)ethyl)isoindoline-1,3-dione A009;
4-(2-(4-(4-(2,6-dioxopiperidin-3-yl)phenyl)-[1,4′:1′,4″-terpiperidin]-1″-yl)ethyl)-2-((S)-1-(3-ethoxy-4-methoxyphenyl)-2-(methylsulfonyl)ethyl)isoindoline-1,3-dione A010;
5-(2-(4-(4-(2,6-dioxopiperidin-3-yl)phenyl)-[1,4′:1′,4″-terpiperidin]-1″-yl)ethyl)-2-((S)-1-(3-ethoxy-4-methoxyphenyl)-2-(methylsulfonyl)ethyl)isoindoline-1,3-dione A011;
5-(4-(1′-(3-(4-(2,6-dioxopiperidin-3-yl)phenyl)propyl)-[1,4′-bipiperidin]-4-yl)-piperazin-1-yl)-2-((S)-1-(3-ethoxy-4-methoxyphenyl)-2-(methylsulfonyl)ethyl)isoindoline-1,3-dione A012;
4-(4-(1′-(3-(4-(2,6-dioxopiperidin-3-yl)phenyl)propyl)-[1,4′-bipiperidin]-4-yl)-piperazin-1-yl)-2-((S)-1-(3-ethoxy-4-methoxyphenyl)-2-(methylsulfonyl)ethyl)isoindoline-1,3-dione A013;
4-(4-(1′-(4-(2,6-dioxopiperidin-3-yl)phenethyl)-[1,4′-bipiperidin]-4-yl)piperazin-1-yl)-2-((S)-1-(3-ethoxy-4-methoxyphenyl)-2-(methylsulfonyl)ethyl)isoindoline-1,3-dione A014;
5-(4-(1′-(4-(2,6-dioxopiperidin-3-yl)phenethyl)-[1,4′-bipiperidin]-4-yl)piperazin-1-yl)-2-((S)-1-(3-ethoxy-4-methoxyphenyl)-2-(methylsulfonyl)ethyl)isoindoline-1,3-dione A015;
4-((1′-(3-(4-(4-(2,6-dioxopiperidin-3-yl)phenyl)piperidin-1-yl)propyl)-[1,4′-bipiperidin]-4-yl)amino)-2-((S)-1-(3-ethoxy-4-methoxyphenyl)-2-(methylsulfonyl)ethyl)-isoindoline-1,3-dione A016;
5-((1′-(3-(4-(4-(2,6-dioxopiperidin-3-yl)phenyl)piperidin-1-yl)propyl)-[1,4′-bipiperidin]-4-yl)amino)-2-((S)-1-(3-ethoxy-4-methoxyphenyl)-2-(methylsulfonyl)ethyl)-isoindoline-1,3-dione A017;
4-((1′-(2-(4-(4-(2,6-dioxopiperidin-3-yl)phenyl)piperidin-1-yl)ethyl)-[1,4′-bipiperidin]-4-yl)amino)-2-((S)-1-(3-ethoxy-4-methoxyphenyl)-2-(methylsulfonyl)ethyl)-isoindoline-1,3-dione A018;
5-((1′-(2-(4-(4-(2,6-dioxopiperidin-3-yl)phenyl)piperidin-1-yl)ethyl)-[1,4′-bipiperidin]-4-yl)amino)-2-((S)-1-(3-ethoxy-4-methoxyphenyl)-2-(methylsulfonyl)ethyl)-isoindoline-1,3-dione A019;
4-(4-(1-(3-(4-(4-(2,6-dioxopiperidin-3-yl)phenyl)piperidin-1-yl)propyl)piperidin-4-yl)piperazin-1-yl)-2-((S)-1-(3-ethoxy-4-methoxyphenyl)-2-(methylsulfonyl)ethyl)isoindoline-1,3-dione A020;
5-(4-(1-(3-(4-(4-(2,6-dioxopiperidin-3-yl)phenyl)piperidin-1-yl)propyl)piperidin-4-yl)piperazin-1-yl)-2-((S)-1-(3-ethoxy-4-methoxyphenyl)-2-(methylsulfonyl)ethyl)isoindoline-1,3-dione A021;
4-(4-(1-(2-(4-(4-(2,6-dioxopiperidin-3-yl)phenyl)piperidin-1-yl)ethyl)piperidin-4-yl)-piperazin-1-yl)-2-((S)-1-(3-ethoxy-4-methoxyphenyl)-2-(methylsulfonyl)ethyl)isoindoline-1,3-dione A022;
5-(4-(1-(2-(4-(4-(2,6-dioxopiperidin-3-yl)phenyl)piperidin-1-yl)ethyl)piperidin-4-yl)piperazin-1-yl)-2-((S)-1-(3-ethoxy-4-methoxyphenyl)-2-(methylsulfonyl)ethyl)isoindoline-1,3-dione A023;
5-(4-(1-((1-(4-(2,6-dioxopiperidin-3-yl)phenyl)piperidin-4-yl)methyl)piperidin-4-yl)piperazin-1-yl)-2-((S)-1-(3-ethoxy-4-methoxyphenyl)-2-(methylsulfonyl)ethyl)isoindoline-1,3-dione A024;
5-(4-(1-(2-(1-(4-(2,6-dioxopiperidin-3-yl)phenyl)piperidin-4-yl)ethyl)azetidin-3-yl)piperazin-1-yl)-2-((S)-1-(3-ethoxy-4-methoxyphenyl)-2-(methylsulfonyl)ethyl)isoindoline-1,3-dione A025;
4-(4-(4-(2,6-dioxopiperidin-3-yl)phenyl)-[1,4′-bipiperidin]-1′-yl)-N-(2-((S)-1-(3-ethoxy-4-methoxyphenyl)-2-(methylsulfonyl)ethyl)-1,3-dioxoisoindolin-4-yl)butanamide A026;
4-(5-(4-(3-(2,6-dioxopiperidin-3-yl)phenyl)-[1,4′-bipiperidin]-1′-yl)pentyl)-2-((S)-1-(3-ethoxy-4-methoxyphenyl)-2-(methylsulfonyl)ethyl)isoindoline-1,3-dione A027;
5-(1-((1′-(4-(2,6-dioxopiperidin-3-yl)phenyl)-[1,4′-bipiperidin]-4-yl)methyl)-piperidin-4-yl)-2-((S)-1-(3-ethoxy-4-methoxyphenyl)-2-(methylsulfonyl)ethyl)isoindoline-1,3-dione A028;
5-(1′-(2-(4-(4-(2,6-dioxopiperidin-3-yl)phenyl)piperidin-1-yl)ethyl)-[4,4′-bipiperidin]-1-yl)-2-((S)-1-(3-ethoxy-4-methoxyphenyl)-2-(methylsulfonyl)ethyl)isoindoline-1,3-dione A029;
5-(1′-(4-(2,6-dioxopiperidin-3-yl)phenyl)-[4,4′-bipiperidin]-1-yl)-2-((S)-1-(3-ethoxy-4-methoxyphenyl)-2-(methylsulfonyl)ethyl)isoindoline-1,3-dione A030;
4-(4-(2-(4-(4-(2,6-dioxopiperidin-3-yl)phenyl)-[1,4′-bipiperidin]-1′-yl)ethyl)-piperidin-1-yl)-2-((S)-1-(3-ethoxy-4-methoxyphenyl)-2-(methylsulfonyl)ethyl)isoindoline-1,3-dione A031;
4-(4-(1-(2-(4-(4-(2,6-dioxopiperidin-3-yl)phenyl)piperazin-1-yl)ethyl)piperidin-4-yl)piperazin-1-yl)-2-((S)-1-(3-ethoxy-4-methoxyphenyl)-2-(methylsulfonyl)ethyl)isoindoline-1,3-dione A032;
5-(4-(1-(2-(4-(4-(2,6-dioxopiperidin-3-yl)phenyl)piperazin-1-yl)ethyl)piperidin-4-yl)piperazin-1-yl)-2-((S)-1-(3-ethoxy-4-methoxyphenyl)-2-(methylsulfonyl)ethyl)isoindoline-1,3-dione A033;
5-(4-(2-(1′-(4-(2,6-dioxopiperidin-3-yl)phenyl)-[4,4′-bipiperidin]-1-yl)ethyl)-piperazin-1-yl)-2-((S)-1-(3-ethoxy-4-methoxyphenyl)-2-(methylsulfonyl)ethyl)isoindoline-1,3-dione A034;
4-(4-(2-(1′-(4-(2,6-dioxopiperidin-3-yl)phenyl)-[4,4′-bipiperidin]-1-yl)ethyl)-piperazin-1-yl)-2-((S)-1-(3-ethoxy-4-methoxyphenyl)-2-(methylsulfonyl)ethyl)isoindoline-1,3-dione A035;
4-((1-(2-(4-(4-(2,6-dioxopiperidin-3-yl)phenyl)piperidin-1-yl)ethyl)piperidin-4-yl)amino)-2-((S)-1-(3-ethoxy-4-methoxyphenyl)-2-(methylsulfonyl)ethyl)isoindoline-1,3-dione A036;
4-((1-((1-(4-(2,6-dioxopiperidin-3-yl)benzyl)piperidin-4-yl)methyl)piperidin-4-yl)amino)-2-((S)-1-(3-ethoxy-4-methoxyphenyl)-2-(methylsulfonyl)ethyl)isoindoline-1,3-dione A037;
5-(4-(4-(4-(3-(2,6-dioxopiperidin-3-yl)phenyl)but-3-yn-1-yl)piperazin-1-yl)-piperidin-1-yl)-2-((S)-1-(3-ethoxy-4-methoxyphenyl)-2-(methylsulfonyl)ethyl)isoindoline-1,3-dione A038;
5-(4-((3-(2,6-dioxopiperidin-3-yl)phenyl)ethynyl)-[1,4′-bipiperidin]-1′-yl)-2-((S)-1-(3-ethoxy-4-methoxyphenyl)-2-(methylsulfonyl)ethyl)isoindoline-1,3-dione A039;
5-(4-(4-(4-(4-(2,6-dioxopiperidin-3-yl)phenyl)but-3-yn-1-yl)piperazin-1-yl)-piperidin-1-yl)-2-((S)-1-(3-ethoxy-4-methoxyphenyl)-2-(methylsulfonyl)ethyl)isoindoline-1,3-dione A040;
5-(4-((4-(2,6-dioxopiperidin-3-yl)phenyl)ethynyl)-[1,4′-bipiperidin]-1′-yl)-2-((S)-1-(3-ethoxy-4-methoxyphenyl)-2-(methylsulfonyl)ethyl)isoindoline-1,3-dione A041;
5-(4-((4-((4-(4-(2,6-dioxopiperidin-3-yl)phenyl)piperidin-1-yl)methyl)piperidin-1-yl)methyl)piperidin-1-yl)-2-((S)-1-(3-ethoxy-4-methoxyphenyl)-2-(methylsulfonyl)ethyl)-isoindoline-1,3-dione A042;
5-((1-((1-(2-(4-(4-(2,6-dioxopiperidin-3-yl)phenyl)piperidin-1-yl)ethyl)piperidin-4-yl)methyl)piperidin-4-yl)amino)-2-((S)-1-(3-ethoxy-4-methoxyphenyl)-2-(methylsulfonyl)-ethyl)isoindoline-1,3-dione A043;
5-(4-((1′-(4-(2,6-dioxopiperidin-3-yl)phenyl)-[4,4′-bipiperidin]-1-yl)methyl)-piperidin-1-yl)-2-((S)-1-(3-ethoxy-4-methoxyphenyl)-2-(methylsulfonyl)ethyl)isoindoline-1,3-dione A044;
5-(4-(3-(4-(2,6-dioxopiperidin-3-yl)phenyl)-3-azaspiro[5.5]undecan-9-yl)-piperazin-1-yl)-2-((S)-1-(3-ethoxy-4-methoxyphenyl)-2-(methylsulfonyl)ethyl)isoindoline-1,3-dione A045;
5-(4-((1-(4-(2,6-dioxopiperidin-3-yl)phenyl)piperidin-4-yl)methyl)piperazin-1-yl)-2-((S)-1-(3-ethoxy-4-methoxyphenyl)-2-(methylsulfonyl)ethyl)isoindoline-1,3-dione A046;
4-(4-(1-(4-(4-(2,6-dioxopiperidin-3-yl)phenyl)cyclohexyl)piperidin-4-yl)-piperazin-1-yl)-2-((S)-1-(3-ethoxy-4-methoxyphenyl)-2-(methylsulfonyl)ethyl)isoindoline-1,3-dione A047;
4-(4-(2-(4-(2,6-dioxopiperidin-3-yl)phenyl)-2-azaspiro[3.3]heptan-6-yl)-piperazin-1-yl)-2-((S)-1-(3-ethoxy-4-methoxyphenyl)-2-(methylsulfonyl)ethyl)isoindoline-1,3-dione A048;
5-(4-(2-(4-(2,6-dioxopiperidin-3-yl)phenyl)-2-azaspiro[3.3]heptan-6-yl)-piperazin-1-yl)-2-((S)-1-(3-ethoxy-4-methoxyphenyl)-2-(methylsulfonyl)ethyl)isoindoline-1,3-dione A049;
4-(4-(1-(3-(2,6-dioxopiperidin-3-yl)phenethyl)piperidin-4-yl)piperazin-1-yl)-2-((S)-1-(3-ethoxy-4-methoxyphenyl)-2-(methylsulfonyl)ethyl)isoindoline-1,3-dione A050;
5-(4-(1-(3-(2,6-dioxopiperidin-3-yl)phenethyl)piperidin-4-yl)piperazin-1-yl)-2-((S)-1-(3-ethoxy-4-methoxyphenyl)-2-(methylsulfonyl)ethyl)isoindoline-1,3-dione A051;
4-(4-((1-(2-(4-(4-(2,6-dioxopiperidin-3-yl)phenyl)piperidin-1-yl)ethyl)piperidin-4-yl)methyl)piperazin-1-yl)-2-((S)-1-(3-ethoxy-4-methoxyphenyl)-2-(methylsulfonyl)ethyl)-isoindoline-1,3-dione A052;
4-((1-((1-(2-(4-(4-(2,6-dioxopiperidin-3-yl)phenyl)piperidin-1-yl)ethyl)piperidin-4-yl)methyl)piperidin-4-yl)amino)-2-((S)-1-(3-ethoxy-4-methoxyphenyl)-2-(methylsulfonyl)-ethyl)isoindoline-1,3-dione A053;
4-(4-(4-(4-(2,6-dioxopiperidin-3-yl)phenyl)cyclohexyl)piperazin-1-yl)-2-((S)-1-(3-ethoxy-4-methoxyphenyl)-2-(methylsulfonyl)ethyl)isoindoline-1,3-dione A054;
5-(4-(4-(2,6-dioxopiperidin-3-yl)phenethyl)piperazin-1-yl)-2-((S)-1-(3-ethoxy-4-methoxyphenyl)-2-(methylsulfonyl)ethyl)isoindoline-1,3-dione A055;
5-(4-(1-(4-(4-(2,6-dioxopiperidin-3-yl)phenyl)cyclohexyl)piperidin-4-yl)-piperazin-1-yl)-2-((S)-1-(3-ethoxy-4-methoxyphenyl)-2-(methylsulfonyl)ethyl)isoindoline-1,3-dione A056;
4-(4-(1-(4-(2,6-dioxopiperidin-3-yl)phenethyl)piperidin-4-yl)piperazin-1-yl)-2-((S)-1-(3-ethoxy-4-methoxyphenyl)-2-(methylsulfonyl)ethyl)isoindoline-1,3-dione A057;
5-(4-(1-(4-(2,6-dioxopiperidin-3-yl)phenethyl)piperidin-4-yl)piperazin-1-yl)-2-((S)-1-(3-ethoxy-4-methoxyphenyl)-2-(methylsulfonyl)ethyl)isoindoline-1,3-dione A058;
5-(1-(4-(4-(4-(2,6-dioxopiperidin-3-yl)phenyl)-[1,4′-bipiperidin]-1′-yl)butyl)-piperidin-4-yl)-2-((1S)-1-(3-ethoxy-4-methoxyphenyl)-2-(methylsulfinyl)ethyl)isoindoline-1,3-dione A059;
5-(1-(2-(4-(4-(2,6-dioxopiperidin-3-yl)phenyl)-[1,4′-bipiperidin]-1′-yl)ethyl)-piperidin-4-yl)-2-((S)-1-(3-ethoxy-4-methoxyphenyl)-2-(methylsulfonyl)ethyl)isoindoline-1,3-dione A060;
5-(1-(3-(4-(4-(2,6-dioxopiperidin-3-yl)phenyl)-[1,4′-bipiperidin]-1′-yl)propyl)-piperidin-4-yl)-2-((S)-1-(3-ethoxy-4-methoxyphenyl)-2-(methylsulfonyl)ethyl)isoindoline-1,3-dione A061;
4-(1-(2-(4-(4-(2,6-dioxopiperidin-3-yl)phenyl)-[1,4′-bipiperidin]-1′-yl)ethyl)-piperidin-4-yl)-2-((S)-1-(3-ethoxy-4-methoxyphenyl)-2-(methylsulfonyl)ethyl)isoindoline-1,3-dione A062;
4-(1-(3-(4-(4-(2,6-dioxopiperidin-3-yl)phenyl)-[1,4′-bipiperidin]-1′-yl)propyl)-piperidin-4-yl)-2-((S)-1-(3-ethoxy-4-methoxyphenyl)-2-(methylsulfonyl)ethyl)isoindoline-1,3-dione A063;
4-(1-(4-(4-(4-(2,6-dioxopiperidin-3-yl)phenyl)-[1,4′-bipiperidin]-1′-yl)butyl)-piperidin-4-yl)-2-((S)-1-(3-ethoxy-4-methoxyphenyl)-2-(methylsulfonyl)ethyl)isoindoline-1,3-dione A064;
5-(5-(4-(4-(2,6-dioxopiperidin-3-yl)phenyl)-[1,4′-bipiperidin]-1′-yl)pentyl)-2-((S)-1-(3-ethoxy-4-methoxyphenyl)-2-(methylsulfonyl)ethyl)isoindoline-1,3-dione A065;
5-(4-(4-(4-(2,6-dioxopiperidin-3-yl)phenyl)-[1,4′-bipiperidin]-1′-yl)butyl)-2-((S)-1-(3-ethoxy-4-methoxyphenyl)-2-(methylsulfonyl)ethyl)isoindoline-1,3-dione A066;
4-(4-(4-(4-(2,6-dioxopiperidin-3-yl)phenyl)-[1,4′-bipiperidin]-1′-yl)butyl)-2-((S)-1-(3-ethoxy-4-methoxyphenyl)-2-(methylsulfonyl)ethyl)isoindoline-1,3-dione A067;
4-(5-(4-(4-(2,6-dioxopiperidin-3-yl)phenyl)-[1,4′-bipiperidin]-1′-yl)pentyl)-2-((S)-1-(3-ethoxy-4-methoxyphenyl)-2-(methylsulfonyl)ethyl)isoindoline-1,3-dione A068;
5-(2-(4-(4-(2,6-dioxopiperidin-3-yl)phenyl)-[1,4′-bipiperidin]-1′-yl)ethyl)-2-((S)-1-(3-ethoxy-4-methoxyphenyl)-2-(methylsulfonyl)ethyl)isoindoline-1,3-dione A069;
5-(3-(4-(4-(2,6-dioxopiperidin-3-yl)phenyl)-[1,4′-bipiperidin]-1′-yl)propyl)-2-((S)-1-(3-ethoxy-4-methoxyphenyl)-2-(methylsulfonyl)ethyl)isoindoline-1,3-dione A070;
4-(2-(4-(4-(2,6-dioxopiperidin-3-yl)phenyl)-[1,4′-bipiperidin]-1′-yl)ethyl)-2-((S)-1-(3-ethoxy-4-methoxyphenyl)-2-(methylsulfonyl)ethyl)isoindoline-1,3-dione A071;
4-(3-(4-(4-(2,6-dioxopiperidin-3-yl)phenyl)-[1,4′-bipiperidin]-1′-yl)propyl)-2-((S)-1-(3-ethoxy-4-methoxyphenyl)-2-(methylsulfonyl)ethyl)isoindoline-1,3-dione A072;
4-(6-(4-(2-(4-(4-(2,6-dioxopiperidin-3-yl)phenyl)piperidin-1-yl)-2-oxoethyl)-piperidin-1-yl)hexyl)-2-((S)-1-(3-ethoxy-4-methoxyphenyl)-2-(methylsulfonyl)ethyl)isoindoline-1,3-dione A073;
4-(11-(4-(3-(2,6-dioxopiperidin-3-yl)phenyl)piperidin-1-yl)undecyl)-2-((S)-1-(3-ethoxy-4-methoxyphenyl)-2-(methylsulfonyl)ethyl)isoindoline-1,3-dione A074;
4-(11-(4-(4-(2,6-dioxopiperidin-3-yl)phenyl)piperidin-1-yl)-11-oxoundecyl)-2-((S)-1-(3-ethoxy-4-methoxyphenyl)-2-(methylsulfonyl)ethyl)isoindoline-1,3-dione A075;
4-(11-(4-(4-(2,6-dioxopiperidin-3-yl)phenyl)piperidin-1-yl)undecyl)-2-((S)-1-(3-ethoxy-4-methoxyphenyl)-2-(methylsulfonyl)ethyl)isoindoline-1,3-dione A076;
5-(1′-(4-(4-(2,6-dioxopiperidin-3-yl)phenoxy)benzyl)-[4,4′-bipiperidin]-1-yl)-2-((S)-1-(3-ethoxy-4-methoxyphenyl)-2-(methylsulfonyl)ethyl)isoindoline-1,3-dione A077;
5-(5-(4-(4-((2,6-dioxopiperidin-3-yl)amino)phenyl)-[1,4′-bipiperidin]-1′-yl)-pentyl)-2-((S)-1-(3-ethoxy-4-methoxyphenyl)-2-(methylsulfonyl)ethyl)isoindoline-1,3-dione A081;
5-((1-((1-(2-(4-(4-((2,6-dioxopiperidin-3-yl)amino)-2-fluoro-5-methoxyphenyl)-piperazin-1-yl)ethyl)piperidin-4-yl)methyl)piperidin-4-yl)amino)-2-((S)-1-(3-ethoxy-4-methoxy-phenyl)-2-(methylsulfonyl)ethyl)isoindoline-1,3-dione A082;
5-(4-(1-((1-(4-((2,6-dioxopiperidin-3-yl)amino)phenyl)piperidin-4-yl)methyl)-piperidin-4-yl)piperazin-1-yl)-2-((S)-1-(3-ethoxy-4-methoxyphenyl)-2-(methylsulfonyl)ethyl)-isoindoline-1,3-dione A083;
5-(4-(2-(4-(4-((2,6-dioxopiperidin-3-yl)amino)phenethyl)piperazin-1-yl)ethyl)-piperidin-1-yl)-2-((S)-1-(3-ethoxy-4-methoxyphenyl)-2-(methylsulfonyl)ethyl)isoindoline-1,3-dione A084;
4-((1-((1-(2-(4-(4-((2,6-dioxopiperidin-3-yl)amino)-2-fluorophenyl)piperidin-1-yl)ethyl)piperidin-4-yl)methyl)piperidin-4-yl)amino)-2-((S)-1-(3-ethoxy-4-methoxyphenyl)-2-(methylsulfonyl)ethyl)isoindoline-1,3-dione A085;
5-(4-((4-((4-(4-((2,6-dioxopiperidin-3-yl)amino)phenyl)piperidin-1-yl)methyl)-piperidin-1-yl)methyl)piperidin-1-yl)-2-((S)-1-(3-ethoxy-4-methoxyphenyl)-2-(methylsulfonyl)-ethyl)isoindoline-1,3-dione A086;
5-(4-((1-(2-((4-((2,6-dioxopiperidin-3-yl)amino)phenyl)amino)ethyl)piperidin-4-yl)methyl)piperazin-1-yl)-2-((S)-1-(3-ethoxy-4-methoxyphenyl)-2-(methylsulfonyl)ethyl)-isoindoline-1,3-dione A087;
5-(1′-(4-(4-((2,6-dioxopiperidin-3-yl)amino)phenoxy)benzyl)-[4,4′-bipiperidin]-1-yl)-2-((S)-1-(3-ethoxy-4-methoxyphenyl)-2-(methylsulfonyl)ethyl)isoindoline-1,3-dione A088;
5-(1′-(4-((3-(2,6-dioxopiperidin-3-yl)-1-methyl-1H-indazol-6-yl)oxy)benzyl)-[4,4′-bipiperidin]-1-yl)-2-((S)-1-(3-ethoxy-4-methoxyphenyl)-2-(methylsulfonyl)ethyl)-isoindoline-1,3-dione B001, 5-(4-((1-(4-((3-(2,6-dioxopiperidin-3-yl)-1-methyl-1H-indazol-6-yl)oxy)benzyl)-piperidin-4-yl)methyl)piperazin-1-yl)-2-((S)-1-(3-ethoxy-4-methoxyphenyl)-2-(methylsulfonyl)-ethyl)isoindoline-1,3-dione B002;
4-(1′-(4-((3-(2,6-dioxopiperidin-3-yl)-1-methyl-1H-indazol-6-yl)oxy)benzyl)-[4,4′-bipiperidin]-1-yl)-2-((S)-1-(3-ethoxy-4-methoxyphenyl)-2-(methylsulfonyl)ethyl)-isoindoline-1,3-dione B003;
5-(1-((1-(4-((3-(2,6-Dioxopiperidin-3-yl)-1-methyl-1H-indazol-6-yl)oxy)benzyl)-piperidin-4-yl)methyl)piperidin-4-yl)-2-((S)-1-(3-ethoxy-4-methoxyphenyl)-2-(methylsulfonyl)-ethyl)isoindoline-1,3-dione B004;
3-(6-(4-((1′-(2-((S)-1-(3-ethoxy-4-methoxyphenyl)-2-(methylsulfonyl)ethyl)-1-oxoisoindolin-5-yl)-[4,4′-bipiperidin]-1-yl)methyl)phenoxy)-1-methyl-1H-indazol-3-yl)-piperidine-2,6-dione B005;
3-(6-(4-((1′-(2-((S)-1-(3-ethoxy-4-methoxyphenyl)-2-(methylsulfonyl)ethyl)-3-oxoisoindolin-5-yl)-[4,4′-bipiperidin]-1-yl)methyl)phenoxy)-1-methyl-1H-indazol-3-yl)-piperidine-2,6-dione B006;
4-(4-((1-(4-((3-(2,6-dioxopiperidin-3-yl)-1-methyl-1H-indazol-6-yl)oxy)benzyl)-piperidin-4-yl)methyl)piperazin-1-yl)-2-((S)-1-(3-ethoxy-4-methoxyphenyl)-2-(methylsulfonyl)-ethyl)isoindoline-1,3-dione B007;
5-((4′R)-1′-(4-((3-(2,6-dioxopiperidin-3-yl)-1-methyl-1H-indazol-6-yl)oxy)-benzyl)-3′,3′-difluoro-[4,4′-bipiperidin]-1-yl)-2-((S)-1-(3-ethoxy-4-methoxyphenyl)-2-(methyl-sulfonyl)ethyl)isoindoline-1,3-dione BOOS;
4-((4′R)-1′-(4-((3-(2,6-dioxopiperidin-3-yl)-1-methyl-1H-indazol-6-yl)oxy)-benzyl)-3′,3′-difluoro-[4,4′-bipiperidin]-1-yl)-2-((S)-1-(3-ethoxy-4-methoxyphenyl)-2-(methyl-sulfonyl)ethyl)isoindoline-1,3-dione B009;
4-(4-((4-(4-((3-(2,6-dioxopiperidin-3-yl)-1-methyl-1H-indazol-6-yl)oxy)phenyl)-piperidin-1-yl)methyl)-4-fluoropiperidin-1-yl)-2-((S)-1-(3-ethoxy-4-methoxyphenyl)-2-(methyl-sulfonyl)ethyl)isoindoline-1,3-dione B010;
4-(1′-(2-(4-((3-(2,6-dioxopiperidin-3-yl)-1-methyl-1H-indazol-6-yl)oxy)phenyl)-2,2-difluoroethyl)-[4,4′-bipiperidin]-1-yl)-2-((S)-1-(3-ethoxy-4-methoxyphenyl)-2-(methyl-sulfonyl)ethyl)isoindoline-1,3-dione B011;
4-(4-(2-(4-(4-((3-(2,6-dioxopiperidin-3-yl)-1-methyl-1H-indazol-6-yl)oxy)-phenyl)piperazin-1-yl)ethyl)piperidin-1-yl)-2-((S)-1-(3-ethoxy-4-methoxyphenyl)-2-(methyl-sulfonyl)ethyl)isoindoline-1,3-dione B012.
5-(1′-(4-((3-((S)-2,6-dioxopiperidin-3-yl)-1-methyl-1H-indazol-6-yl)oxy)benzyl)-[4,4′-bipiperidin]-1-yl)-2-((S)-1-(3-ethoxy-4-methoxyphenyl)-2-(methylsulfonyl)ethyl)-isoindoline-1,3-dione B021;
5-(1′-(4-((3-((R)-2,6-dioxopiperidin-3-yl)-1-methyl-1H-indazol-6-yl)oxy)benzyl)-[4,4′-bipiperidin]-1-yl)-2-((S)-1-(3-ethoxy-4-methoxyphenyl)-2-(methylsulfonyl)ethyl)-isoindoline-1,3-dione B022;
5-(4-((S)-3,3-difluoro-1-(4-((1-methyl-3-((S)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)isoindoline-1,3-dione B023;
5-(4-((S)-3,3-difluoro-1-(4-((1-methyl-3-((R)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)isoindoline-1,3-dione B024;
5-(4-((S)-3,3-difluoro-1-(4-((5-fluoro-1-methyl-3-((S)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)isoindoline-1,3-dione B025;
5-(4-((S)-3,3-difluoro-1-(4-((5-fluoro-1-methyl-3-((R)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)isoindoline-1,3-dione B026;
5-(4-((R)-3,3-difluoro-1-(4-((1-methyl-3-((S)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)isoindoline-1,3-dione B027;
5-(4-((R)-3,3-difluoro-1-(4-((1-methyl-3-((R)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)isoindoline-1,3-dione B028;
5-(4-((R)-3,3-difluoro-1-(4-((5-fluoro-1-methyl-3-((S)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)isoindoline-1,3-dione B029;
5-(4-((R)-3,3-difluoro-1-(4-((5-fluoro-1-methyl-3-((R)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)isoindoline-1,3-dione B030;
4-(4-((S)-3,3-difluoro-1-(4-((1-methyl-3-((S)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)isoindoline-1,3-dione B031;
4-(4-((S)-3,3-difluoro-1-(4-((1-methyl-3-((R)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)isoindoline-1,3-dione B032;
4-(4-((S)-3,3-difluoro-1-(4-((5-fluoro-1-methyl-3-((S)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)isoindoline-1,3-dione B033;
4-(4-((S)-3,3-difluoro-1-(4-((5-fluoro-1-methyl-3-((S)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)isoindoline-1,3-dione B034;
4-(4-((R)-3,3-difluoro-1-(4-((1-methyl-3-((S)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)isoindoline-1,3-dione B035;
4-(4-((R)-3,3-difluoro-1-(4-((1-methyl-3-((R)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)isoindoline-1,3-dione B036;
4-(4-((R)-3,3-difluoro-1-(4-((5-fluoro-1-methyl-3-((S)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)isoindoline-1,3-dione B037;
4-(4-((R)-3,3-difluoro-1-(4-((5-fluoro-1-methyl-3-((S)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)isoindoline-1,3-dione B038;
4-(1′-(2-(4-((3-((R)-2,6-dioxopiperidin-3-yl)-1-methyl-1H-indazol-6-yl)oxy)-phenyl)-2,2-difluoroethyl)-[4,4′-bipiperidin]-1-yl)-2-((S)-1-(3-ethoxy-4-methoxyphenyl)-2-(methylsulfonyl)ethyl)isoindoline-1,3-dione B039;
4-(1′-(2-(4-((3-((S)-2,6-dioxopiperidin-3-yl)-1-methyl-1H-indazol-6-yl)oxy)-phenyl)-2,2-difluoroethyl)-[4,4′-bipiperidin]-1-yl)-2-((S)-1-(3-ethoxy-4-methoxyphenyl)-2-(methylsulfonyl)ethyl)isoindoline-1,3-dione B040;
4-(4-(1-(2,2-difluoro-2-(4-((1-methyl-3-((S)-3-methyl-2,6-dioxopiperidin-3-yl)-1H-indazol-6-yl)oxy)phenyl)ethyl)piperidin-4-yl)piperazin-1-yl)-2-((S)-1-(3-ethoxy-4-methoxyphenyl)-2-(methylsulfonyl)ethyl)isoindoline-1,3-dione B041;
4-(4-(1-(2,2-difluoro-2-(4-((1-methyl-3-((R)-3-methyl-2,6-dioxopiperidin-3-yl)-1H-indazol-6-yl)oxy)phenyl)ethyl)piperidin-4-yl)piperazin-1-yl)-2-((S)-1-(3-ethoxy-4-methoxyphenyl)-2-(methylsulfonyl)ethyl)isoindoline-1,3-dione B042;
4-(4-(1-(2,2-difluoro-2-(4-((5-fluoro-1-methyl-3-((S)-3-methyl-2,6-dioxo-piperidin-3-yl)-1H-indazol-6-yl)oxy)phenyl)ethyl)piperidin-4-yl)piperazin-1-yl)-2-((S)-1-(3-ethoxy-4-methoxyphenyl)-2-(methylsulfonyl)ethyl)isoindoline-1,3-dione B043;
4-(4-(1-(2,2-Difluoro-2-(4-((5-fluoro-1-methyl-3-((R)-3-methyl-2,6-dioxo-piperidin-3-yl)-1H-indazol-6-yl)oxy)phenyl)ethyl)piperidin-4-yl)piperazin-1-yl)-2-((S)-1-(3-ethoxy-4-methoxyphenyl)-2-(methylsulfonyl)ethyl)isoindoline-1,3-dione B044;
4-(4-(2-(4-(4-((3-((S)-2,6-dioxopiperidin-3-yl)-1-methyl-1H-indazol-6-yl)oxy)-phenyl)piperazin-1-yl)ethyl)piperidin-1-yl)-2-((S)-1-(3-ethoxy-4-methoxyphenyl)-2-(methyl-sulfonyl)ethyl)isoindoline-1,3-dione B045;
4-(4-(2-(4-(4-((3-((R)-2,6-dioxopiperidin-3-yl)-1-methyl-1H-indazol-6-yl)oxy)-phenyl)piperazin-1-yl)ethyl)piperidin-1-yl)-2-((S)-1-(3-ethoxy-4-methoxyphenyl)-2-(methyl-sulfonyl)ethyl)isoindoline-1,3-dione B046;
2-((S)-1-(3-ethoxy-4-methoxyphenyl)-2-(methylsulfonyl)ethyl)-4-(4-(2-(1-(4-((1-methyl-3-((R)-3-methyl-2,6-dioxopiperidin-3-yl)-1H-indazol-6-yl)oxy)phenyl)piperidin-4-yl)-ethyl)piperazin-1-yl)isoindoline-1,3-dione B047;
2-((S)-1-(3-ethoxy-4-methoxyphenyl)-2-(methylsulfonyl)ethyl)-4-(4-(2-(1-(4-((5-fluoro-1-methyl-3-((R)-3-methyl-2,6-dioxopiperidin-3-yl)-1H-indazol-6-yl)oxy)phenyl)-piperidin-4-yl)ethyl)piperazin-1-yl)isoindoline-1,3-dione B048;
4-((4'S)-1′-(4-((3-(2,6-dioxopiperidin-3-yl)-1-methyl-1H-indazol-6-yl)oxy)phen-ethyl)-3′,3′-difluoro-[4,4′-bipiperidin]-1-yl)-2-((S)-1-(3-ethoxy-4-methoxyphenyl)-2-(methyl-sulfonyl)ethyl)isoindoline-1,3-dione B049;
6-(4-((S)-3,3-difluoro-1-(4-((1-methyl-3-((R)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B051;
6-(4-((S)-3,3-difluoro-1-(4-((5-fluoro-1-methyl-3-((R)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B052;
6-(4-((R)-3,3-difluoro-1-(4-((1-methyl-3-((R)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B053;
6-(4-((R)-3,3-difluoro-1-(4-((5-fluoro-1-methyl-3-((R)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B054;
4-(4-((S)-3,3-difluoro-1-(4-((1-methyl-3-((R)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B055;
4-(4-((S)-3,3-difluoro-1-(4-((5-fluoro-1-methyl-3-((R)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B056;
4-(4-((R)-3,3-difluoro-1-(4-((1-methyl-3-((R)-3-methyl-2,6-dioxopiperidin-3-yl)-1H-indazol-6-yl)oxy)benzyl)piperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B057;
4-(4-((R)-3,3-difluoro-1-(4-((5-fluoro-1-methyl-3-((R)-3-methyl-2,6-dioxo-piperidin-3-yl)-1H-indazol-6-yl)oxy)benzyl)piperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B058;
4-(4-(1-(2,2-difluoro-2-(4-((1-methyl-3-((S)-3-methyl-2,6-dioxopiperidin-3-yl)-1H-indazol-6-yl)oxy)phenyl)ethyl)piperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B059;
4-(4-(1-(2,2-difluoro-2-(4-((1-methyl-3-((R)-3-methyl-2,6-dioxopiperidin-3-yl)-1H-indazol-6-yl)oxy)phenyl)ethyl)piperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B060;
4-(4-(1-(2,2-difluoro-2-(4-((5-fluoro-1-methyl-3-((R)-3-methyl-2,6-dioxo-piperidin-3-yl)-1H-indazol-6-yl)oxy)phenyl)ethyl)piperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B061;
5-((R)-1′-(4-((3-(2,4-dioxotetrahydropyrimidin-1(2H)-yl)-1-methyl-1H-indazol-6-yl)oxy)benzyl)-3′,3′-difluoro-[4,4′-bipiperidin]-1-yl)-2-((S)-1-(3-ethoxy-4-methoxyphenyl)-2-(methylsulfonyl)ethyl)isoindoline-1,3-dione B151;
4-((R)-1′-(4-((3-(2,4-dioxotetrahydropyrimidin-1(2H)-yl)-5-fluoro-1-methyl-1H-indazol-6-yl)oxy)benzyl)-3′,3′-difluoro-[4,4′-bipiperidin]-1-yl)-2-((S)-1-(3-ethoxy-4-methoxy-phenyl)-2-(methylsulfonyl)ethyl)isoindoline-1,3-dione B152;
4-(4-((R)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)isoindoline-1,3-dione B153;
5-((R)-1′-(4-((3-(2,4-dioxotetrahydropyrimidin-1(2H)-yl)-5-fluoro-1-methyl-1H-indazol-6-yl)oxy)benzyl)-3′,3′-difluoro-[4,4′-bipiperidin]-1-yl)-2-((S)-1-(3-ethoxy-4-methoxy-phenyl)-2-(methylsulfonyl)ethyl)isoindoline-1,3-dione B154;
5-(4-((S)-1-(4-((3-(2,4-dioxotetrahydropyrimidin-1(2H)-yl)-1-methyl-1H-indazol-6-yl)oxy)benzyl)-3,3-difluoropiperidin-4-yl)piperazin-1-yl)-2-((S)-1-(3-ethoxy-4-methoxy-phenyl)-2-(methylsulfonyl)ethyl)isoindoline-1,3-dione B155;
5-(4-((R)-1-(4-((3-(2,4-dioxotetrahydropyrimidin-1(2H)-yl)-1-methyl-1H-indazol-6-yl)oxy)benzyl)-3,3-difluoropiperidin-4-yl)piperazin-1-yl)-2-((S)-1-(3-ethoxy-4-methoxy-phenyl)-2-(methylsulfonyl)ethyl)isoindoline-1,3-dione B156;
5-(4-((S)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)isoindoline-1,3-dione B157;
5-(4-((R)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)isoindoline-1,3-dione B158;
4-((R)-1′-(4-((3-(2,4-dioxotetrahydropyrimidin-1(2H)-yl)-1-methyl-1H-indazol-6-yl)oxy)benzyl)-3′,3′-difluoro-[4,4′-bipiperidin]-1-yl)-2-((S)-1-(3-ethoxy-4-methoxyphenyl)-2-(methylsulfonyl)ethyl)isoindoline-1,3-dione B159;
4-(4-((S)-1-(4-((3-(2,4-dioxotetrahydropyrimidin-1(2H)-yl)-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)isoindoline-1,3-dione B160;
4-(4-((S)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)isoindoline-1,3-dione B161;
4-(4-((R)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)isoindoline-1,3-dione B162;
(S)-4-(4-((4-(4-((3-(2,4-dioxotetrahydropyrimidin-1(2H)-yl)-1-methyl-1H-indazol-6-yl)oxy)phenyl)piperidin-1-yl)methyl)-4-fluoropiperidin-1-yl)-2-(1-(3-ethoxy-4-methoxyphenyl)-2-(methylsulfonyl)ethyl)isoindoline-1,3-dione B163;
(S)-4-(4-((4-(4-((3-(2,4-dioxotetrahydropyrimidin-1(2H)-yl)-5-fluoro-1-methyl-1H-indazol-6-yl)oxy)phenyl)piperidin-1-yl)methyl)-4-fluoropiperidin-1-yl)-2-(1-(3-ethoxy-4-methoxyphenyl)-2-(methylsulfonyl)ethyl)isoindoline-1,3-dione B164;
(S)-5-(4-((4-(4-((3-(2,4-dioxotetrahydropyrimidin-1(2H)-yl)-1-methyl-1H-indazol-6-yl)oxy)phenyl)piperidin-1-yl)methyl)-4-fluoropiperidin-1-yl)-2-(1-(3-ethoxy-4-methoxyphenyl)-2-(methylsulfonyl)ethyl)isoindoline-1,3-dione B165;
(S)-5-(4-((4-(4-((3-(2,4-dioxotetrahydropyrimidin-1(2H)-yl)-5-fluoro-1-methyl-1H-indazol-6-yl)oxy)phenyl)piperidin-1-yl)methyl)-4-fluoropiperidin-1-yl)-2-(1-(3-ethoxy-4-methoxyphenyl)-2-(methylsulfonyl)ethyl)isoindoline-1,3-dione B166;
(S)-4-(1′-(2-(4-((3-(2,4-dioxotetrahydropyrimidin-1(2H)-yl)-1-methyl-1H-indazol-6-yl)oxy)phenyl)-2,2-difluoroethyl)-[4,4′-bipiperidin]-1-yl)-2-(1-(3-ethoxy-4-methoxy-phenyl)-2-(methylsulfonyl)ethyl)isoindoline-1,3-dione B167;
(S)-4-(1′-(2-(4-((3-(2,4-dioxotetrahydropyrimidin-1(2H)-yl)-5-fluoro-1-methyl-1H-indazol-6-yl)oxy)phenyl)-2,2-difluoroethyl)-[4,4′-bipiperidin]-1-yl)-2-(1-(3-ethoxy-4-methoxyphenyl)-2-(methylsulfonyl)ethyl)isoindoline-1,3-dione B168;
(S)-4-(4-(1-(2-(4-((3-(2,4-dioxotetrahydropyrimidin-1(2H)-yl)-1-methyl-1H-indazol-6-yl)oxy)phenyl)-2,2-difluoroethyl)piperidin-4-yl)piperazin-1-yl)-2-(1-(3-ethoxy-4-methoxyphenyl)-2-(methylsulfonyl)ethyl)isoindoline-1,3-dione B169;
(S)-4-(4-(1-(2-(4-((3-(2,4-dioxotetrahydropyrimidin-1(2H)-yl)-5-fluoro-1-methyl-1H-indazol-6-yl)oxy)phenyl)-2,2-difluoroethyl)piperidin-4-yl)piperazin-1-yl)-2-(1-(3-ethoxy-4-methoxyphenyl)-2-(methylsulfonyl)ethyl)isoindoline-1,3-dione B170;
(S)-4-(4-(2-(4-(4-((3-(2,4-dioxotetrahydropyrimidin-1(2H)-yl)-1-methyl-1H-indazol-6-yl)oxy)phenyl)piperazin-1-yl)ethyl)piperidin-1-yl)-2-(1-(3-ethoxy-4-methoxyphenyl)-2-(methylsulfonyl)ethyl)isoindoline-1,3-dione B171;
(S)-4-(4-(2-(4-(4-((3-(2,4-dioxotetrahydropyrimidin-1(2H)-yl)-5-fluoro-1-methyl-1H-indazol-6-yl)oxy)phenyl)piperazin-1-yl)ethyl)piperidin-1-yl)-2-(1-(3-ethoxy-4-methoxyphenyl)-2-(methylsulfonyl)ethyl)isoindoline-1,3-dione B172;
(S)-4-(4-(2-(1-(4-((3-(2,4-dioxotetrahydropyrimidin-1(2H)-yl)-1-methyl-1H-indazol-6-yl)oxy)phenyl)piperidin-4-yl)ethyl)piperazin-1-yl)-2-(1-(3-ethoxy-4-methoxyphenyl)-2-(methylsulfonyl)ethyl)isoindoline-1,3-dione B173;
(S)-4-(4-(2-(1-(4-((3-(2,4-dioxotetrahydropyrimidin-1(2H)-yl)-5-fluoro-1-methyl-1H-indazol-6-yl)oxy)phenyl)piperidin-4-yl)ethyl)piperazin-1-yl)-2-(1-(3-ethoxy-4-methoxy-phenyl)-2-(methylsulfonyl)ethyl)isoindoline-1,3-dione B174;
4-((S)-1′-(4-((3-(2,4-dioxotetrahydropyrimidin-1(2H)-yl)-1-methyl-1H-indazol-6-yl)oxy)phenethyl)-3′,3′-difluoro-[4,4′-bipiperidin]-1-yl)-2-((S)-1-(3-ethoxy-4-methoxyphenyl)-2-(methylsulfonyl)ethyl)isoindoline-1,3-dione B175;
4-((S)-1′-(4-((3-(2,4-dioxotetrahydropyrimidin-1(2H)-yl)-5-fluoro-1-methyl-1H-indazol-6-yl)oxy)phenethyl)-3′,3′-difluoro-[4,4′-bipiperidin]-1-yl)-2-((S)-1-(3-ethoxy-4-methoxy-phenyl)-2-(methylsulfonyl)ethyl)isoindoline-1,3-dione B176;
4-(4-((R)-1-(4-((3-(2,4-dioxotetrahydropyrimidin-1(2H)-yl)-1-methyl-1H-indazol-6-yl)oxy)phenethyl)-3,3-difluoropiperidin-4-yl)piperazin-1-yl)-2-((S)-1-(3-ethoxy-4-methoxy-phenyl)-2-(methylsulfonyl)ethyl)isoindoline-1,3-dione B177;
4-(4-((R)-1-(4-((3-(2,4-dioxotetrahydropyrimidin-1(2H)-yl)-5-fluoro-1-methyl-1H-indazol-6-yl)oxy)phenethyl)-3,3-difluoropiperidin-4-yl)piperazin-1-yl)-2-((S)-1-(3-ethoxy-4-methoxyphenyl)-2-(methylsulfonyl)ethyl)isoindoline-1,3-dione B178;
4-(4-((S)-1-(4-((3-(2,4-dioxotetrahydropyrimidin-1(2H)-yl)-1-methyl-1H-indazol-6-yl)oxy)phenethyl)-3,3-difluoropiperidin-4-yl)piperazin-1-yl)-2-((S)-1-(3-ethoxy-4-methoxy-phenyl)-2-(methylsulfonyl)ethyl)isoindoline-1,3-dione B179;
4-(4-((S)-1-(4-((3-(2,4-dioxotetrahydropyrimidin-1(2H)-yl)-5-fluoro-1-methyl-1H-indazol-6-yl)oxy)phenethyl)-3,3-difluoropiperidin-4-yl)piperazin-1-yl)-2-((S)-1-(3-ethoxy-4-methoxyphenyl)-2-(methylsulfonyl)ethyl)isoindoline-1,3-dione B180;
6-((R)-1′-(4-((3-(2,4-dioxotetrahydropyrimidin-1(2H)-yl)-1-methyl-1H-indazol-6-yl)oxy)benzyl)-3′,3′-difluoro-[4,4′-bipiperidin]-1-yl)-2-((S)-1-(3-ethoxy-4-methoxyphenyl)-2-(methylsulfonyl)ethyl)-1H-pyrrolo[3,4-c]pyridine-1,3(2H)-dione B201;
6-((R)-1′-(4-((3-(2,4-dioxotetrahydropyrimidin-1(2H)-yl)-5-fluoro-1-methyl-1H-indazol-6-yl)oxy)benzyl)-3′,3′-difluoro-[4,4′-bipiperidin]-1-yl)-2-((S)-1-(3-ethoxy-4-methoxy-phenyl)-2-(methylsulfonyl)ethyl)-1H-pyrrolo[3,4-c]pyridine-1,3(2H)-dione B202;
6-(4-((S)-1-(4-((3-(2,4-dioxotetrahydropyrimidin-1(2H)-yl)-1-methyl-1H-indazol-6-yl)oxy)benzyl)-3,3-difluoropiperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B203;
6-(4-((S)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B204;
6-(4-((R)-1-(4-((3-(2,4-dioxotetrahydropyrimidin-1(2H)-yl)-1-methyl-1H-indazol-6-yl)oxy)benzyl)-3,3-difluoropiperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B205;
6-(4-((R)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B206;
4-((R)-1′-(4-((3-(2,4-dioxotetrahydropyrimidin-1(2H)-yl)-1-methyl-1H-indazol-6-yl)oxy)benzyl)-3′,3′-difluoro-[4,4′-bipiperidin]-1-yl)-2-((S)-1-(3-ethoxy-4-methoxyphenyl)-2-(methylsulfonyl)ethyl)-1H-pyrrolo[3,4-c]pyridine-1,3(2H)-dione B207;
4-((R)-1′-(4-((3-(2,4-dioxotetrahydropyrimidin-1(2H)-yl)-5-fluoro-1-methyl-1H-indazol-6-yl)oxy)benzyl)-3′,3′-difluoro-[4,4′-bipiperidin]-1-yl)-2-((S)-1-(3-ethoxy-4-methoxy-phenyl)-2-(methylsulfonyl)ethyl)-1H-pyrrolo[3,4-c]pyridine-1,3(2H)-dione B208;
4-(4-((S)-1-(4-((3-(2,4-dioxotetrahydropyrimidin-1(2H)-yl)-1-methyl-1H-indazol-6-yl)oxy)benzyl)-3,3-difluoropiperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B209;
4-(4-((S)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B210;
4-(4-((R)-1-(4-((3-(2,4-dioxotetrahydropyrimidin-1(2H)-yl)-1-methyl-1H-indazol-6-yl)oxy)benzyl)-3,3-difluoropiperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B211;
4-(4-((R)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B212;
(S)-4-(4-(1-(2-(4-((3-(2,4-dioxotetrahydropyrimidin-1(2H)-yl)-5-fluoro-1-methyl-1H-indazol-6-yl)oxy)phenyl)-2,2-difluoroethyl)piperidin-4-yl)piperazin-1-yl)-2-(1-(3-ethoxy-4-methoxyphenyl)-2-(methylsulfonyl)ethyl)-1H-pyrrolo[3,4-c]pyridine-1,3(2H)-dione B213;
(S)-4-(4-(2-(4-(4-((3-(2,4-dioxotetrahydropyrimidin-1(2H)-yl)-1-methyl-1H-indazol-6-yl)oxy)phenyl)piperazin-1-yl)ethyl)piperidin-1-yl)-2-(1-(3-ethoxy-4-methoxyphenyl)-2-(methylsulfonyl)ethyl)-1H-pyrrolo[3,4-c]pyridine-1,3(2H)-dione B214;
(S)-4-(4-(2-(4-(4-((3-(2,4-dioxotetrahydropyrimidin-1(2H)-yl)-5-fluoro-1-methyl-1H-indazol-6-yl)oxy)phenyl)piperazin-1-yl)ethyl)piperidin-1-yl)-2-(1-(3-ethoxy-4-methoxy-phenyl)-2-(methylsulfonyl)ethyl)-1H-pyrrolo[3,4-c]pyridine-1,3(2H)-dione B215;
(S)-4-(4-(2-(1-(4-((3-(2,4-dioxotetrahydropyrimidin-1(2H)-yl)-1-methyl-1H-indazol-6-yl)oxy)phenyl)piperidin-4-yl)ethyl)piperazin-1-yl)-2-(1-(3-ethoxy-4-methoxyphenyl)-2-(methylsulfonyl)ethyl)-1H-pyrrolo[3,4-c]pyridine-1,3(2H)-dione B216;
(S)-4-(4-(2-(1-(4-((3-(2,4-dioxotetrahydropyrimidin-1(2H)-yl)-5-fluoro-1-methyl-1H-indazol-6-yl)oxy)phenyl)piperidin-4-yl)ethyl)piperazin-1-yl)-2-(1-(3-ethoxy-4-methoxy-phenyl)-2-(methylsulfonyl)ethyl)-1H-pyrrolo[3,4-c]pyridine-1,3(2H)-dione B217;
4-((S)-1′-(4-((3-(2,4-dioxotetrahydropyrimidin-1(2H)-yl)-1-methyl-1H-indazol-6-yl)oxy)phenethyl)-3′,3′-difluoro-[4,4′-bipiperidin]-1-yl)-2-((S)-1-(3-ethoxy-4-methoxyphenyl)-2-(methylsulfonyl)ethyl)-1H-pyrrolo[3,4-c]pyridine-1,3(2H)-dione B218;
4-((S)-1′-(4-((3-(2,4-dioxotetrahydropyrimidin-1(2H)-yl)-5-fluoro-1-methyl-1H-indazol-6-yl)oxy)phenethyl)-3′,3′-difluoro-[4,4′-bipiperidin]-1-yl)-2-((S)-1-(3-ethoxy-4-methoxy-phenyl)-2-(methylsulfonyl)ethyl)-1H-pyrrolo[3,4-c]pyridine-1,3(2H)-dione B219;
4-(4-((R)-1-(4-((3-(2,4-dioxotetrahydropyrimidin-1(2H)-yl)-1-methyl-1H-indazol-6-yl)oxy)phenethyl)-3,3-difluoropiperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B220;
4-(4-((R)-1-(4-((3-(2,4-dioxotetrahydropyrimidin-1(2H)-yl)-5-fluoro-1-methyl-1H-indazol-6-yl)oxy)phenethyl)-3,3-difluoropiperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B221;
4-(4-((S)-1-(4-((3-(2,4-dioxotetrahydropyrimidin-1(2H)-yl)-1-methyl-1H-indazol-6-yl)oxy)phenethyl)-3,3-difluoropiperidin-4-yl)piperazin-1-yl)-2-((S)-1-(3-ethoxy-4-methoxy-phenyl)-2-(methylsulfonyl)ethyl)-1H-pyrrolo[3,4-c]pyridine-1,3(2H)-dione B222;
4-(4-((S)-1-(4-((3-(2,4-dioxotetrahydropyrimidin-1(2H)-yl)-5-fluoro-1-methyl-1H-indazol-6-yl)oxy)phenethyl)-3,3-difluoropiperidin-4-yl)piperazin-1-yl)-2-((S)-1-(3-ethoxy-4-methoxyphenyl)-2-(methylsulfonyl)ethyl)-1H-pyrrolo[3,4-c]pyridine-1,3(2H)-dione B223.
(3R)-3-(5-(4-((4-((4-(4-(2,6-dioxopiperidin-3-yl)phenyl)piperidin-1-yl)methyl)-piperidin-1-yl)methyl)piperidin-1-yl)-1,3-dioxoisoindolin-2-yl)-3-(3-ethoxy-4-methoxyphenyl)-propanenitrile C001;
(3R)-3-(5-(4-((4-((4-(4-((2,6-dioxopiperidin-3-yl)amino)phenyl)piperidin-1-yl)-methyl)piperidin-1-yl)methyl)piperidin-1-yl)-1,3-dioxoisoindolin-2-yl)-3-(3-ethoxy-4-methoxy-phenyl)propanenitrile C002;
(3R)-3-(6-(1′-(4-((3-(2,6-dioxopiperidin-3-yl)-1-methyl-1H-indazol-6-yl)oxy)-benzyl)-[4,4′-bipiperidin]-1-yl)-1-oxoisoindolin-2-yl)-3-(3-ethoxy-4-methoxyphenyl)propanenitrile C011;
(3R)-3-(5-(1′-(4-((3-(2,6-dioxopiperidin-3-yl)-1-methyl-1H-indazol-6-yl)oxy)-benzyl)-[4,4′-bipiperidin]-1-yl)-1,3-dioxoisoindolin-2-yl)-3-(3-ethoxy-4-methoxyphenyl)-propanenitrile C012;
(3R)-3-(6-(1′-(4-((3-(2,6-dioxopiperidin-3-yl)-1-methyl-1H-indazol-6-yl)oxy)-benzyl)-[4,4′-bipiperidin]-1-yl)-1-oxoisoindolin-2-yl)-3-(3-ethoxy-4-methoxyphenyl)propanenitrile C013;
(3R)-3-(5-(1′-(4-((3-(2,6-dioxopiperidin-3-yl)-1-methyl-1H-indazol-6-yl)oxy)-benzyl)-[4,4′-bipiperidin]-1-yl)-1-oxoisoindolin-2-yl)-3-(3-ethoxy-4-methoxyphenyl)propanenitrile C014;
(3R)-3-(4-(4-((1-(4-((3-(2,6-dioxopiperidin-3-yl)-1-methyl-1H-indazol-6-yl)oxy)-benzyl)piperidin-4-yl)methyl)piperazin-1-yl)-1,3-dioxoisoindolin-2-yl)-3-(3-ethoxy-4-methoxy-phenyl)propanenitrile C015;
(3R)-3-(5-(4-((1-(4-((3-(2,6-dioxopiperidin-3-yl)-1-methyl-1H-indazol-6-yl)oxy)-benzyl)piperidin-4-yl)methyl)piperazin-1-yl)-1,3-dioxoisoindolin-2-yl)-3-(3-ethoxy-4-methoxy-phenyl)propanenitrile C016;
(3R)-3-(5-(1-((1-(4-((3-(2,6-dioxopiperidin-3-yl)-1-methyl-1H-indazol-6-yl)oxy)-benzyl)piperidin-4-yl)methyl)piperidin-4-yl)-1,3-dioxoisoindolin-2-yl)-3-(3-ethoxy-4-methoxy-phenyl)propanenitrile C017;
(3R)-3-(4-(1′-(4-((3-(2,6-dioxopiperidin-3-yl)-1-methyl-1H-indazol-6-yl)oxy)-benzyl)-[4,4′-bipiperidin]-1-yl)-1,3-dioxoisoindolin-2-yl)-3-(3-ethoxy-4-methoxyphenyl)-propanenitrile C018;
(3R)-3-(5-(4-((4-((4-(3-(2,6-dioxopiperidin-3-yl)-1-methyl-1H-indazol-6-yl)-piperidin-1-yl)methyl)piperidin-1-yl)methyl)piperidin-1-yl)-1,3-dioxoisoindolin-2-yl)-3-(3-ethoxy-4-methoxyphenyl)propanenitrile C019;
(3R)-3-(4-((1-((1-(2-(4-(3-(2,6-dioxopiperidin-3-yl)-1-methyl-1H-indazol-6-yl)-piperazin-1-yl)ethyl)piperidin-4-yl)methyl)piperidin-4-yl)amino)-1,3-dioxoisoindolin-2-yl)-3-(3-ethoxy-4-methoxyphenyl)propanenitrile C020;
(3R)-3-(4-(4-((1-(2-(4-(3-(2,6-dioxopiperidin-3-yl)-1-methyl-1H-indazol-6-yl)-piperidin-1-yl)acetyl)piperidin-4-yl)methyl)piperazin-1-yl)-1,3-dioxoisoindolin-2-yl)-3-(3-ethoxy-4-methoxyphenyl)propanenitrile C021;
(3R)-3-(5-(4-((4-((3-(2,6-dioxopiperidin-3-yl)-1-methyl-1H-indazol-6-yl)oxy)-piperidin-1-yl)methyl)-[1,4′-bipiperidin]-1′-yl)-1,3-dioxoisoindolin-2-yl)-3-(3-ethoxy-4-methoxyphenyl)propanenitrile C022;
2-(4-(11-(2-((R)-2-cyano-1-(3-ethoxy-4-methoxyphenyl)ethyl)-1,3-dioxo-isoindolin-4-yl)undecyl)piperazin-1-yl)-N-(3-(2,6-dioxopiperidin-3-yl)-1-methyl-1H-indazol-6-yl)acetamide C023;
(R)-3-(6-(4-((S)-3,3-difluoro-1-(4-((1-methyl-3-((R)-3-methyl-2,6-dioxo-piperidin-3-yl)-1H-indazol-6-yl)oxy)benzyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C031;
(R)-3-(6-(4-((S)-3,3-difluoro-1-(4-((5-fluoro-1-methyl-3-((R)-3-methyl-2,6-dioxopiperidin-3-yl)-1H-indazol-6-yl)oxy)benzyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C032;
(R)-3-(6-(4-((R)-3,3-difluoro-1-(4-((1-methyl-3-((R)-3-methyl-2,6-dioxo-piperidin-3-yl)-1H-indazol-6-yl)oxy)benzyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C033;
(R)-3-(6-(4-((R)-3,3-difluoro-1-(4-((5-fluoro-1-methyl-3-((R)-3-methyl-2,6-dioxopiperidin-3-yl)-1H-indazol-6-yl)oxy)benzyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C034;
(R)-3-(4-(4-((S)-3,3-difluoro-1-(4-((1-methyl-3-((R)-3-methyl-2,6-dioxo-piperidin-3-yl)-1H-indazol-6-yl)oxy)benzyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C035;
(R)-3-(4-(4-((S)-3,3-difluoro-1-(4-((5-fluoro-1-methyl-3-((R)-3-methyl-2,6-dioxopiperidin-3-yl)-1H-indazol-6-yl)oxy)benzyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C036;
(R)-3-(4-(4-((R)-3,3-difluoro-1-(4-((1-methyl-3-((R)-3-methyl-2,6-dioxo-piperidin-3-yl)-1H-indazol-6-yl)oxy)benzyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C037;
(R)-3-(4-(4-((R)-3,3-difluoro-1-(4-((5-fluoro-1-methyl-3-((R)-3-methyl-2,6-dioxopiperidin-3-yl)-1H-indazol-6-yl)oxy)benzyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C038;
(R)-3-(4-(4-(1-(2,2-difluoro-2-(4-((1-methyl-3-((R)-3-methyl-2,6-dioxopiperidin-3-yl)-1H-indazol-6-yl)oxy)phenyl)ethyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C039;
(R)-3-(4-(4-(1-(2,2-difluoro-2-(4-((5-fluoro-1-methyl-3-((R)-3-methyl-2,6-dioxo-piperidin-3-yl)-1H-indazol-6-yl)oxy)phenyl)ethyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C040;
(R)-3-(3-ethoxy-4-methoxyphenyl)-3-(4-(4-(2-(1-(4-((1-methyl-3-((R)-3-methyl-2,6-dioxopiperidin-3-yl)-1H-indazol-6-yl)oxy)phenyl)piperidin-4-yl)ethyl)piperazin-1-yl)-1,3-dioxo-1,3-dihydro-2H-pyrrolo[3,4-c]pyridin-2-yl)propanenitrile C041;
(R)-3-(3-ethoxy-4-methoxyphenyl)-3-(4-(4-(2-(1-(4-((5-fluoro-1-methyl-3-((R)-3-methyl-2,6-dioxopiperidin-3-yl)-1H-indazol-6-yl)oxy)phenyl)piperidin-4-yl)ethyl)piperazin-1-yl)-1,3-dioxo-1,3-dihydro-2H-pyrrolo[3,4-c]pyridin-2-yl)propanenitrile C042;
(R)-3-(5-(4-((S)-1-(4-((3-(2,4-dioxotetrahydropyrimidin-1(2H)-yl)-1-methyl-1H-indazol-6-yl)oxy)benzyl)-3,3-difluoropiperidin-4-yl)piperazin-1-yl)-1,3-dioxoisoindolin-2-yl)-3-(3-ethoxy-4-methoxyphenyl)propanenitrile C051;
(R)-3-(5-(4-((R)-1-(4-((3-(2,4-dioxotetrahydropyrimidin-1(2H)-yl)-1-methyl-1H-indazol-6-yl)oxy)benzyl)-3,3-difluoropiperidin-4-yl)piperazin-1-yl)-1,3-dioxoisoindolin-2-yl)-3-(3-ethoxy-4-methoxyphenyl)propanenitrile C052;
(R)-3-(5-(4-((S)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-1,3-dioxo-isoindolin-2-yl)-3-(3-ethoxy-4-methoxyphenyl)propanenitrile C053;
(R)-3-(5-(4-((R)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-1,3-dioxo-isoindolin-2-yl)-3-(3-ethoxy-4-methoxyphenyl)propanenitrile C054;
(R)-3-(4-(4-((S)-1-(4-((3-(2,4-dioxotetrahydropyrimidin-1(2H)-yl)-1-methyl-1H-indazol-6-yl)oxy)benzyl)-3,3-difluoropiperidin-4-yl)piperazin-1-yl)-1,3-dioxoisoindolin-2-yl)-3-(3-ethoxy-4-methoxyphenyl)propanenitrile C055;
(R)-3-(4-(4-((S)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-1,3-dioxo-isoindolin-2-yl)-3-(3-ethoxy-4-methoxyphenyl)propanenitrile C056;
(R)-3-(4-(4-((R)-1-(4-((3-(2,4-dioxotetrahydropyrimidin-1(2H)-yl)-1-methyl-1H-indazol-6-yl)oxy)benzyl)-3,3-difluoropiperidin-4-yl)piperazin-1-yl)-1,3-dioxoisoindolin-2-yl)-3-(3-ethoxy-4-methoxyphenyl)propanenitrile C057;
(R)-3-(4-(4-((R)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-1,3-dioxo-isoindolin-2-yl)-3-(3-ethoxy-4-methoxyphenyl)propanenitrile C058;
(R)-3-(4-(4-((4-(4-((3-(2,4-dioxotetrahydropyrimidin-1(2H)-yl)-1-methyl-1H-indazol-6-yl)oxy)phenyl)piperidin-1-yl)methyl)-4-fluoropiperidin-1-yl)-1,3-dioxoisoindolin-2-yl)-3-(3-ethoxy-4-methoxyphenyl)propanenitrile C059;
(R)-3-(4-(4-((4-(4-((3-(2,4-dioxotetrahydropyrimidin-1(2H)-yl)-5-fluoro-1-methyl-1H-indazol-6-yl)oxy)phenyl)piperidin-1-yl)methyl)-4-fluoropiperidin-1-yl)-1,3-dioxo-isoindolin-2-yl)-3-(3-ethoxy-4-methoxyphenyl)propanenitrile C060;
(R)-3-(5-(4-((4-(4-((3-(2,4-dioxotetrahydropyrimidin-1(2H)-yl)-1-methyl-1H-indazol-6-yl)oxy)phenyl)piperidin-1-yl)methyl)-4-fluoropiperidin-1-yl)-1,3-dioxoisoindolin-2-yl)-3-(3-ethoxy-4-methoxyphenyl)propanenitrile C061;
(R)-3-(5-(4-((4-(4-((3-(2,4-dioxotetrahydropyrimidin-1(2H)-yl)-5-fluoro-1-methyl-1H-indazol-6-yl)oxy)phenyl)piperidin-1-yl)methyl)-4-fluoropiperidin-1-yl)-1,3-dioxoisoindolin-2-yl)-3-(3-ethoxy-4-methoxyphenyl)propanenitrile C062;
(R)-3-(4-(4-(1-(2-(4-((3-(2,4-dioxotetrahydropyrimidin-1(2H)-yl)-1-methyl-1H-indazol-6-yl)oxy)phenyl)-2,2-difluoroethyl)piperidin-4-yl)piperazin-1-yl)-1,3-dioxoisoindolin-2-yl)-3-(3-ethoxy-4-methoxyphenyl)propanenitrile C063;
(R)-3-(4-(4-(1-(2-(4-((3-(2,4-dioxotetrahydropyrimidin-1(2H)-yl)-5-fluoro-1-methyl-1H-indazol-6-yl)oxy)phenyl)-2,2-difluoroethyl)piperidin-4-yl)piperazin-1-yl)-1,3-dioxo-isoindolin-2-yl)-3-(3-ethoxy-4-methoxyphenyl)propanenitrile C064;
(R)-3-(4-(4-(2-(4-(4-((3-(2,4-dioxotetrahydropyrimidin-1(2H)-yl)-1-methyl-1H-indazol-6-yl)oxy)phenyl)piperazin-1-yl)ethyl)piperidin-1-yl)-1,3-dioxoisoindolin-2-yl)-3-(3-ethoxy-4-methoxyphenyl)propanenitrile C065;
(R)-3-(4-(4-(2-(4-(4-((3-(2,4-dioxotetrahydropyrimidin-1(2H)-yl)-5-fluoro-1-methyl-1H-indazol-6-yl)oxy)phenyl)piperazin-1-yl)ethyl)piperidin-1-yl)-1,3-dioxoisoindolin-2-yl)-3-(3-ethoxy-4-methoxyphenyl)propanenitrile C066;
(R)-3-(4-(4-(2-(1-(4-((3-(2,4-dioxotetrahydropyrimidin-1(2H)-yl)-1-methyl-1H-indazol-6-yl)oxy)phenyl)piperidin-4-yl)ethyl)piperazin-1-yl)-1,3-dioxoisoindolin-2-yl)-3-(3-ethoxy-4-methoxyphenyl)propanenitrile C067;
(R)-3-(4-(4-(2-(1-(4-((3-(2,4-dioxotetrahydropyrimidin-1(2H)-yl)-5-fluoro-1-methyl-1H-indazol-6-yl)oxy)phenyl)piperidin-4-yl)ethyl)piperazin-1-yl)-1,3-dioxoisoindolin-2-yl)-3-(3-ethoxy-4-methoxyphenyl)propanenitrile C068;
(R)-3-(4-(4-((R)-1-(4-((3-(2,4-dioxotetrahydropyrimidin-1(2H)-yl)-1-methyl-1H-indazol-6-yl)oxy)phenethyl)-3,3-difluoropiperidin-4-yl)piperazin-1-yl)-1,3-dioxoisoindolin-2-yl)-3-(3-ethoxy-4-methoxyphenyl)propanenitrile C069;
(R)-3-(4-(4-((R)-1-(4-((3-(2,4-dioxotetrahydropyrimidin-1(2H)-yl)-5-fluoro-1-methyl-1H-indazol-6-yl)oxy)phenethyl)-3,3-difluoropiperidin-4-yl)piperazin-1-yl)-1,3-dioxo-isoindolin-2-yl)-3-(3-ethoxy-4-methoxyphenyl)propanenitrile C070;
(R)-3-(4-(4-((S)-1-(4-((3-(2,4-dioxotetrahydropyrimidin-1(2H)-yl)-1-methyl-1H-indazol-6-yl)oxy)phenethyl)-3,3-difluoropiperidin-4-yl)piperazin-1-yl)-1,3-dioxoisoindolin-2-yl)-3-(3-ethoxy-4-methoxyphenyl)propanenitrile C071;
(R)-3-(4-(4-((S)-1-(4-((3-(2,4-dioxotetrahydropyrimidin-1(2H)-yl)-5-fluoro-1-methyl-1H-indazol-6-yl)oxy)phenethyl)-3,3-difluoropiperidin-4-yl)piperazin-1-yl)-1,3-dioxo-isoindolin-2-yl)-3-(3-ethoxy-4-methoxyphenyl)propanenitrile C072;
(R)-3-(6-((R)-1′-(4-((3-(2,4-dioxotetrahydropyrimidin-1(2H)-yl)-1-methyl-1H-indazol-6-yl)oxy)benzyl)-3′,3′-difluoro-[4,4′-bipiperidin]-1-yl)-1,3-dioxo-1,3-dihydro-2H-pyrrolo[3,4-c]pyridin-2-yl)-3-(3-ethoxy-4-methoxyphenyl)propanenitrile C101;
(R)-3-(6-((R)-1′-(4-((3-(2,4-dioxotetrahydropyrimidin-1(2H)-yl)-5-fluoro-1-methyl-1H-indazol-6-yl)oxy)benzyl)-3′,3′-difluoro-[4,4′-bipiperidin]-1-yl)-1,3-dioxo-1,3-dihydro-2H-pyrrolo[3,4-c]pyridin-2-yl)-3-(3-ethoxy-4-methoxyphenyl)propanenitrile C102;
(R)-3-(6-(4-((S)-1-(4-((3-(2,4-dioxotetrahydropyrimidin-1(2H)-yl)-1-methyl-1H-indazol-6-yl)oxy)benzyl)-3,3-difluoropiperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C103;
(R)-3-(6-(4-((S)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C104;
(R)-3-(6-(4-((R)-1-(4-((3-(2,4-dioxotetrahydropyrimidin-1(2H)-yl)-1-methyl-1H-indazol-6-yl)oxy)benzyl)-3,3-difluoropiperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C105;
(R)-3-(6-(4-((R)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C106;
(R)-3-(4-((R)-1′-(4-((3-(2,4-dioxotetrahydropyrimidin-1(2H)-yl)-1-methyl-1H-indazol-6-yl)oxy)benzyl)-3′,3′-difluoro-[4,4′-bipiperidin]-1-yl)-1,3-dioxo-1,3-dihydro-2H-pyrrolo[3,4-c]pyridin-2-yl)-3-(3-ethoxy-4-methoxyphenyl)propanenitrile C107;
(R)-3-(4-((R)-1′-(4-((3-(2,4-dioxotetrahydropyrimidin-1(2H)-yl)-5-fluoro-1-methyl-1H-indazol-6-yl)oxy)benzyl)-3′,3′-difluoro-[4,4′-bipiperidin]-1-yl)-1,3-dioxo-1,3-dihydro-2H-pyrrolo[3,4-c]pyridin-2-yl)-3-(3-ethoxy-4-methoxyphenyl)propanenitrile C108;
(R)-3-(4-(4-((S)-1-(4-((3-(2,4-dioxotetrahydropyrimidin-1(2H)-yl)-1-methyl-1H-indazol-6-yl)oxy)benzyl)-3,3-difluoropiperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C109;
(R)-3-(4-(4-((S)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C110;
(R)-3-(4-(4-((R)-1-(4-((3-(2,4-dioxotetrahydropyrimidin-1(2H)-yl)-1-methyl-1H-indazol-6-yl)oxy)benzyl)-3,3-difluoropiperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C111;
(R)-3-(4-(4-((R)-1-(4-((3-(2,4-dioxotetrahydropyrimidin-1(2H)-yl)-5-fluoro-1-methyl-1H-indazol-6-yl)oxy)benzyl)-3,3-difluoropiperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C112;
(R)-3-(4-(4-(1-(2-(4-((3-(2,4-dioxotetrahydropyrimidin-1(2H)-yl)-1-methyl-1H-indazol-6-yl)oxy)phenyl)-2,2-difluoroethyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C113;
(R)-3-(4-(4-(1-(2-(4-((3-(2,4-dioxotetrahydropyrimidin-1(2H)-yl)-5-fluoro-1-methyl-1H-indazol-6-yl)oxy)phenyl)-2,2-difluoroethyl)piperidin-4-yl)piperazin-1-yl)-1,3-dioxo-1,3-dihydro-2H-pyrrolo[3,4-c]pyridin-2-yl)-3-(3-ethoxy-4-methoxyphenyl)propanenitrile C114;
(R)-3-(4-(4-(2-(4-(4-((3-(2,4-dioxotetrahydropyrimidin-1(2H)-yl)-1-methyl-1H-indazol-6-yl)oxy)phenyl)piperazin-1-yl)ethyl)piperidin-1-yl)-1,3-dioxo-1,3-dihydro-2H-pyrrolo-[3,4-c]pyridin-2-yl)-3-(3-ethoxy-4-methoxyphenyl)propanenitrile C115;
(R)-3-(4-(4-(2-(4-(4-((3-(2,4-dioxotetrahydropyrimidin-1(2H)-yl)-5-fluoro-1-methyl-1H-indazol-6-yl)oxy)phenyl)piperazin-1-yl)ethyl)piperidin-1-yl)-1,3-dioxo-1,3-dihydro-2H-pyrrolo[3,4-c]pyridin-2-yl)-3-(3-ethoxy-4-methoxyphenyl)propanenitrile C116;
(R)-3-(4-(4-(2-(1-(4-((3-(2,4-dioxotetrahydropyrimidin-1(2H)-yl)-1-methyl-1H-indazol-6-yl)oxy)phenyl)piperidin-4-yl)ethyl)piperazin-1-yl)-1,3-dioxo-1,3-dihydro-2H-pyrrolo-[3,4-c]pyridin-2-yl)-3-(3-ethoxy-4-methoxyphenyl)propanenitrile C117;
(R)-3-(4-(4-(2-(1-(4-((3-(2,4-dioxotetrahydropyrimidin-1(2H)-yl)-5-fluoro-1-methyl-1H-indazol-6-yl)oxy)phenyl)piperidin-4-yl)ethyl)piperazin-1-yl)-1,3-dioxo-1,3-dihydro-2H-pyrrolo[3,4-c]pyridin-2-yl)-3-(3-ethoxy-4-methoxyphenyl)propanenitrile C118;
5-(5-(4-(1-(2,6-dioxopiperidin-3-yl)indolin-6-yl)-[1,4′-bipiperidin]-1′-yl)pentyl)-2-((S)-1-(3-ethoxy-4-methoxyphenyl)-2-(methylsulfonyl)ethyl)isoindoline-1,3-dione D001;
5-(4-((1′-(3-(2,6-dioxopiperidin-3-yl)-1-methyl-2-oxo-2,3-dihydro-1H-benzo[d]-imidazol-5-yl)-[4,4′-bipiperidin]-1-yl)methyl)piperidin-1-yl)-2-((S)-1-(3-ethoxy-4-methoxy-phenyl)-2-(methylsulfonyl)ethyl)isoindoline-1,3-dione D002;
5-((1-((1-((1-(3-(2,6-dioxopiperidin-3-yl)-1-methyl-2-oxo-2,3-dihydro-1H-benzo[d]imidazol-5-yl)azetidin-3-yl)methyl)piperidin-4-yl)methyl)piperidin-4-yl)amino)-2-((S)-1-(3-ethoxy-4-methoxyphenyl)-2-(methylsulfonyl)ethyl)isoindoline-1,3-dione D003;
or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof.

73. A pharmaceutical composition comprising a compound of any one of claims 1 to 72, or a pharmaceutically acceptable salt thereof, and a pharmaceutically acceptable excipient.

74. The pharmaceutical composition of claim 73, wherein the composition is in single dosage form.

75. The pharmaceutical composition of claim 73 or 74, wherein the composition is in an oral, parenteral, or intravenous dosage form.

76. The pharmaceutical composition of claim 75, wherein the composition is formulated in an oral dosage form.

77. The pharmaceutical composition of claim 76, wherein the oral dosage form is a tablet or capsule.

78. A method of treating, preventing, or ameliorating one or more symptoms of a disease, disorder, or condition mediated by a PDE4 in a subject, comprising administering to the subject in need thereof a therapeutically effective amount of a compound of any one of claims 1 to 72 or a pharmaceutical composition of any one of claims 73 to 77.

79. The method of claim 78, wherein the disease, disorder, or condition associated with a PDE4 is an inflammatory disease.

80. A method of treating, preventing, or ameliorating one or more symptoms of an inflammatory disease in a subject, comprising administering to the subject in need thereof a therapeutically effective amount of a compound of any one of claims 1 to 72 or a pharmaceutical composition of any one of claims 73 to 77.

81. The method of claim 79 or 80, wherein the inflammatory disease is arthritis, ankylosing spondylitis, osteoarthritis, rheumatoid arthritis, Behcet's disease, inflammatory bowel disease, psoriasis, psoriatic arthritis, atopic dermatitis, or contact dermatitis, chronic or obstructive pulmonary disease (COPD).

82. The method of any one of claims 78 to 81, wherein the compound is administered orally or topically.

83. The method of any one of claims 78 to 82, wherein the subject is a human.

84. A method of inhibiting the activity of a PDE4, comprising contacting the PDE4 with an effective amount of a compound of any one of claims 1 to 72 or a pharmaceutical composition of any one of claims 73 to 77.

85. A method of inducing degradation of a PDE4, comprising contacting the PDE4 with an effective amount of a compound of any one of claims 1 to 72 or a pharmaceutical composition of any one of claims 73 to 77.

86. The method of claim 84 or 85, wherein the PDE4 is PDE4D.
